(19) United States
(12) Patent Application Publication
(10) Pub. No.: US 2024/0123079 A1
(43) Pub. Date: Apr. 18, 2024

(54) IMMUNOMODULATORY ANTIBODY-DRUG CONJUGATES

(71) Applicant: Seagen Inc., Bothell, WA (US)

(72) Inventors: Adam G. Hill, Seattle, WA (US); Elizabeth E. Gray, Seattle, WA (US); Elizabeth J. Cummins, Granite Falls, WA (US); Patrick J. Burke, Seattle, WA (US); Shyra J. Gardai, Monroe, WA (US)

(21) Appl. No.: 18/222,313

(22) Filed: Jul. 14, 2023

Related U.S. Application Data
(63) Continuation of application No. PCT/US2022/012599, filed on Jan. 14, 2022.
(60) Provisional application No. 63/138,360, filed on Jan. 15, 2021.
(60) Provisional application No. 63/292,779, filed on Dec. 22, 2021.

(51) Int. Cl. A61K 47/68; A61P 35/00; C07D 403/14; C07D 405/14; C07D 409/14; C07D 413/14; C07D 417/14; C07H 15/22

(52) U.S. Cl. CPC A61K 47/6803; A61P 35/00; C07D 403/14; C07D 405/14; C07D 409/14; C07D 413/14; C07D 417/14; C07H 15/22

(57) ABSTRACT

The present disclosure provides, inter alia, antibody-drug conjugates that are useful in treating various diseases such as cancer.

### CROSS REFERENCE TO RELATED APPLICATIONS

This application is a continuation of PCT Application No. PCT/US2022/012599 filed Jan. 14, 2022, which claims the benefit of U.S. Provisional Application Nos. 63/138,360, filed Jan. 15, 2021, and 63/292,779 filed Dec. 22, 2021, both of which are incorporated herein by reference in their entireties.

### BACKGROUND

### Field of the Invention

The present invention relates to the fields of chemistry and medicine. More particularly, the present invention relates to antibody-drug conjugates, compositions, their preparation, and their use as therapeutic agents.

### Description of the Related Art

The cGAS-STING pathway is an innate immune pathway that recognizes intracellular DNA and triggers a type I interferon and inflammatory cytokine response that is important for both anti-viral and anti-tumor immunity. Upon DNA binding, cGMP-AMP synthase (cGAS) produces cGAMP, which is the endogenous ligand of STING. See, e.g., Villanueva, Nat. Rev. Drug Disc. 2019: 18; 15. At the molecular level, upon activation by cGAMP, the transmembrane STING dimer translocates from the endoplasmic reticulum to the Golgi apparatus, ultimately recruiting TANK-binding kinase 1 (TBK1) and the transcription factor interferon regulatory factor 3 (IRF3), leading to induction of type I interferons (IFNs) and an inflammatory response. See Konno, et al., Cell 2013: 155; 688-698. This innate immune pathway must be tightly regulated as excessive cGAS-STING activity has been linked to various autoimmune and inflammatory disorders. See Barber, Nat. Rev. Immunol. 2015: 15; 760-770; see also, Liu, et al., N. Engl. J. Med. 2014: 371; 507-518.

Exogenous STING agonists can help to overcome the immunosuppressive tumor microenvironment by activating an immune response against a tumor, resulting in tumor regression. See Sun, et al., Science 2013: 6121; 786-791; see also, Corrales and Gajewski, Clinc. Cancer Res. 2015: 21; 4774-4779. Examples include nucleotide-based STING agonists, which are, like the endogenous ligands, cyclic di-nucleotides. These compounds are typically charged and hydrophilic, susceptible to enzymatic degradation, and have poor bioavailability and pharmacokinetics. Thus, there remains a need for STING agonists with improved pharmacological properties that avoid systemic cytokine induction.

### SUMMARY

Some embodiments described herein relate to antibody-drug conjugates (ADCs) that can elicit a localized immune response to target cells, and hence, exhibit reduced off-target toxicity, such as that observed with systemically administered immunostimutory compounds.

Some embodiments provide an antibody-drug conjugate (ADC) comprising: an antibody;
a linker, as described herein; and
a compound of Formula (I) as described herein;
wherein the compound of Formula (I) is conjugated to the linker; and
wherein each linker is conjugated to the antibody via a succinimide or hydrolyzed
succinimide covalently linked to a sulfur atom of a cysteine residue of the antibody.

Some embodiments provide an antibody-drug conjugate (ADC) having the formula:

Ab-(S*-M¹-(D))ₚ

wherein:
Ab is an antibody;
each S* is a sulfur atom from a cysteine residue of the antibody;
M¹ is a succinimide or a hydrolyzed succinimide;
subscript p is an integer from 2 to 8; and
each (D) is a Drug Unit of Formula (I), as described herein.

In some embodiments, Formula (I) has the structure:

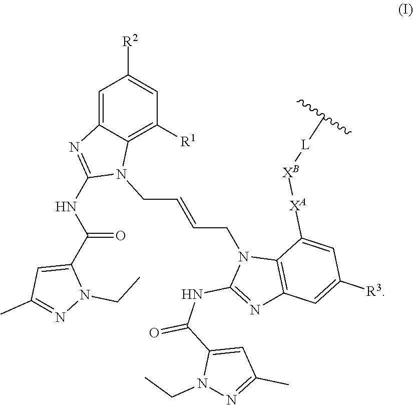

Some embodiments provide a compound of Formula (II):

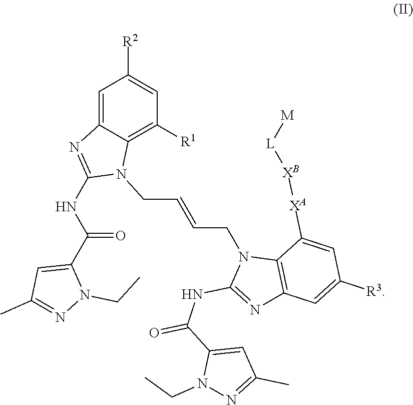

Some embodiments provide an antibody-drug conjugate (ADC) having the formula:

Ab-(S*-(D′))ₚ

wherein:
Ab is an antibody;
each S* is a sulfur atom from a cysteine residue of the antibody;
D′ is a drug unit that is a radical of the compound of Formula (IV), as described herein; and
subscript p is an integer from 2 to 8.

In some embodiments, Formula (IV) has the structure:

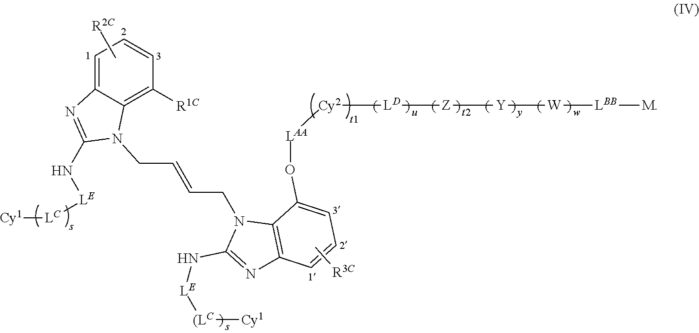

Some embodiments provide a compound of Formula (III):

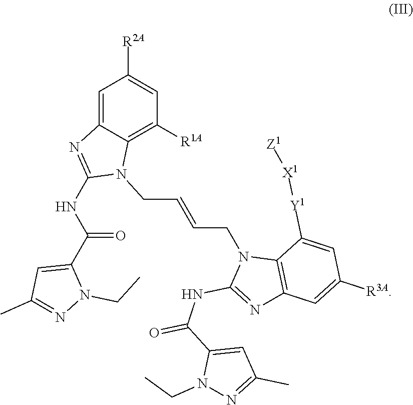

Some embodiments provide a compound having the structure of Formula (V):

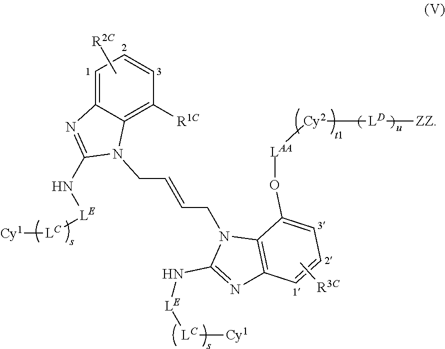

Some embodiments provide a composition comprising a distribution of ADCs as described herein.

Some embodiments provide a method of treating cancer in a subject in need thereof, comprising administering a therapeutically effective amount of an ADC composition, as described herein, to the subject.

Some embodiments provide a method of treating cancer in a subject in need thereof, comprising administering a therapeutically effective amount of an ADC, as described herein, to the subject.

Some embodiments provide a method of inducing an anti-tumor immune response in a subject in need thereof, comprising administering a therapeutically effective amount of an ADC composition, as described herein, to the subject.

Some embodiments provide a method of inducing an anti-tumor immune response in a subject in need thereof, comprising administering a therapeutically effective amount of an ADC, as described herein, to the subject.

### BRIEF DESCRIPTION OF THE DRAWINGS

FIG. 1 illustrates the response of THP1-Dual™ cells (also referred to as THP1 dual reporter cells) to various small molecule STING agonists.

FIG. 2 illustrates the response of wild type (WT) and STING-deficient murine bone marrow-derived macrophages to various small molecule STING agonists.

FIG. 3 illustrates the response of THP1 dual reporter cells to ADCs comprising a non-targeted or targeted antibody conjugated to either compound 11 (cleavable linker with compound 1), compound 12 (non-cleavable linker with compound 12a), or compounds 13 or 14 (cleavable linkers with compound 12a).

FIG. 4 illustrates the response of THP1 dual reporter cells to compound 12 (non-cleavable linker with compound 12a) and compound 16 (cysteine adduct of compound 12 and free drug released from ADCs containing compound 12).

FIG. 5 illustrates the response of THP1 dual reporter cells to compounds 12a and 15b as a free drug or conjugated to a non-binding or targeted antibody (ADC of compounds 12 and 15) following incubation for 48 hours.

FIGS. 6A and 6B illustrate the response of SU-DHL-1 lymphoma cells to ADCs comprising a non-targeted, antigen C-targeted or PD-L1-targeted antibody conjugated to compound 11 (cleavable linker with compound 1). Both cytokine production (MIP-1α) (FIG. 6A) and viability (FIG. 6B) are plotted.

FIG. 7 illustrates the response of THP1 dual reporter cells cultured alone or co-cultured with HEK 293T cells engineered to express target antigen C to ADCs comprising an antigen C-targeted mAb with a hIgG1 LALAPG backbone conjugated to compounds 12, 13, or 14.

FIG. 8 illustrates the bystander activity of ADCs comprising either an EphA2-targeted mAb or a non-binding mAb with a mIgG2a WT or LALAPG backbone conjugated to compound 12 using Renca cancer cells and THP1 dual reporter cells.

FIGS. 9A and 9B illustrate the response to q7dx3 ADC dosing (3 weekly doses) in a Renca tumor mouse model to evaluate various ADCs comprising a non-binding or EphA2-targeted mAb with a mIgG2a LALAPG backbone conjugated to compound 11 (dosed intraperitoneally), or compound 1 or (E)-1-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide tris(2,2,2-trifluoroacetate) (Compound A, a reference compound, dosed intravenously). FIG. 9A: tumor growth; FIG. 9B: % weight change.

FIGS. 10A and 10B illustrate the response to q7dx3 ADC dosing (3 weekly doses) in a Renca tumor mouse model to evaluate various ADCs comprising a non-binding or EphA2-targeted mAb with a mIgG2a LALAPG backbone conjugated to compounds 11 or 12 (dosed intraperitoneally). FIG. 10A: tumor growth; FIG. 10B: % weight change.

FIGS. 11A and 11B illustrate the response to q7dx3 ADC dosing (3 weekly doses) in a Renca tumor mouse model, which is engineered to express a human protein, to evaluate various ADCs comprising a non-binding or EphA2-targeted mAb with either a mIgG2a wild type (WT) or a mIgG2a LALAPG backbone conjugated to compounds 12 or 15. FIG. 11A: tumor growth; FIG. 11B: % weight change.

FIG. 12 illustrates the response to q7dx3 dosing (3 weekly doses, intraperitoneally) in a Renca tumor mouse model to evaluate the ADC comprising an EphA2-targeted mAb with a mIgG2a LALAPG backbone conjugated to compound 12 or unconjugated compound 12a.

FIG. 13 illustrates the response to q7dx3 dosing (3 weekly doses) of various compounds in a Renca tumor model to evaluate PD-L1-targeted mAb, and various ADCs comprising a non-binding, PD-L1-targeted or antigen C-targeted mAb conjugated to compound 11.

FIG. 14 illustrates the response to q7dx3 dosing (3 weekly doses) of various compounds in a CT26 tumor model to evaluate unconjugated compound 1, a PD-L1-targeted mAb, and various ADCs comprising a non-binding, antigen C, PD-L1, or EphA2-targeted mAb conjugated to compound 11.

FIGS. 15A-D illustrate the response to q7dx3 (3 weekly doses) or a single dose of ADC, as indicated, in a MC38 tumor model to evaluate various ADCs comprising a non-binding or EphA2-targeted mAb with a mIgG2a LALAPG backbone conjugated to compound 12. Mice that achieved complete tumor regression in response to ADC treatment were rechallenged with MC38 tumor cells and tumor growth was monitored. FIG. 15A: tumor growth (WT mice); FIG. 15B: weight loss (WT mice); FIG. 15C: tumor growth (STING-deficient Tmem173^{gt} mice); FIG. 15D: tumor growth following MC38 tumor rechallenge.

FIGS. 16A and 16B illustrate the response to q7dx3 mAb or ADC dosing (3 weekly doses indicated by the arrow heads) in a 4T1 tumor model to evaluate various ADCs comprising a non-binding or EphA2-targeted mAb with a mIgG2a LALAPG backbone conjugated to compound 12. FIG. 16A: tumor growth; FIG. 16B: % weight change.

FIG. 17 illustrates the pharmacokinetic profile of an ADC comprising a [deglycosylated] non-binding mAb conjugated to compound 12 following administration to male C57BL/6 mice.

### DETAILED DESCRIPTION

Provided herein are antibody-drug conjugates (ADCs) that can elicit a localized immune response to target cells, and hence, reduced off-target toxicity, for example, as compared to the toxicity often observed with systemic administration of immunostimutory compounds, such as STING agonists. The in vivo toxicity of such compounds is often linked to systemic cytokine activation, resulting in both on- and off-target immune responses. The ADCs described herein include STING agonists as the drug payload to provide localized, selective induction of cytokines. See, e.g., Milling, et al., Adv. Drug Deliv. Rev. 2017: 114; 79-101; see also, Hu, et al., EBioMedicine 2019: 41; 497-508. This approach can deliver specific STING activation, as well as localized immune cell recruitment, while reducing systemic cytokine release and its concomitant adverse effects.

### Definitions

Unless otherwise defined, all technical and scientific terms used herein have the same meaning as commonly understood by one of ordinary skill in the art to which this disclosure belongs. Methods and materials are described herein for use in the present application; other, suitable methods and materials known in the art in some aspects of this disclosure are also used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control. When trade names are used herein, the trade name includes the product formulation, the generic drug, and the active pharmaceutical ingredient(s) of the trade name product, unless otherwise indicated by context.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a linker” includes reference to one or more such linkers, and reference to “the cell” includes reference to a plurality of such cells.

The term “about” when referring to a number or a numerical range means that the number or numerical range referred to is an approximation, for example, within experimental variability and/or statistical experimental error, and thus the number or numerical range may vary up to ±10% of the stated number or numerical range. In reference to an ADC composition comprising a distribution of ADCs as described herein, the average number of conjugated STING agonist compounds to an antibody in the composition can be an integer or a non-integer, particularly when the antibody is to be partially loaded. Thus, the term “about” recited prior to an average drug loading value is intended to capture the expected variations in drug loading within an ADC composition.

The term “antibody” as used herein covers intact monoclonal antibodies, polyclonal antibodies, monospecific antibodies, multispecific antibodies (e.g., bispecific antibodies), including intact antibodies and antigen binding antibody fragments, and reduced forms thereof in which one or more of the interchain disulfide bonds are disrupted, that exhibit the desired biological activity and provided that the antigen binding antibody fragments have the requisite number of attachment sites for the desired number of attached groups, such as a linker (L), as described herein. In some aspects, the linkers are attached via a succinimide or hydrolyzed succinimide to the sulfur atoms of cysteine residues of reduced interchain disulfide bonds and/or cysteine residues introduced by genetic engineering. The native form of an antibody is a tetramer and consists of two identical pairs of immunoglobulin chains, each pair having one light chain and one heavy chain. In each pair, the light and heavy chain variable domains (VL and VH) are together primarily responsible for binding to an antigen. The light chain and heavy chain variable domains consist of a framework region interrupted by three hypervariable regions, also called “complementarity determining regions” or “CDRs.” The light chain and heavy chains also contain constant regions that may be recognized by and interact with the immune system. (see, e.g., Janeway et al., 2001, Immuno. Biology, 5^{th} Ed., Garland Publishing, New York). An antibody includes any isotype (e.g., IgG, IgE, IgM, IgD, and IgA) or subclass (e.g., IgG1, IgG2, IgG3, IgG4, IgA1 and IgA2) thereof. The antibody is derivable from any suitable species. In some aspects, the antibody is of human or murine origin, and in some aspects the antibody is a human, humanized or chimeric antibody. Antibodies can be fucosylated to varying extents or afucosylated.

An “intact antibody” is one which comprises an antigen-binding variable region as well as light chain constant domains (CL) and heavy chain constant domains, C_{H}1, C_{H}2, C_{H}3 and C_{H}4, as appropriate for the antibody class. The constant domains are either native sequence constant domains (e.g., human native sequence constant domains) or amino acid sequence variants thereof.

An “antibody fragment” comprises a portion of an intact antibody, comprising the antigen-binding or variable region thereof. Antibody fragments of the present disclosure include at least one cysteine residue (natural or engineered) that provides a site for attachment of a linker and/or linker-drug compound. In some embodiments, an antibody fragment includes Fab, Fab′, or F(ab′)₂.

As used herein the term “engineered cysteine residue” or “eCys residue” refers to a cysteine amino acid or a derivative thereof that is incorporated into an antibody. In those aspects one or more eCys residues can be incorporated into an antibody, and typically, the eCys residues are incorporated into either the heavy chain or the light chain of an antibody. Generally, incorporation of an eCys residue into an antibody is performed by mutagenizing a nucleic acid sequence of a parent antibody to encode for one or more amino acid residues with a cysteine or a derivative thereof. Suitable mutations include replacement of a desired residue in the light or heavy chain of an antibody with a cysteine or a derivative thereof, incorporation of an additional cysteine or a derivative thereof at a desired location in the light or heavy chain of an antibody, as well as adding an additional cysteine or a derivative thereof to the N- and/or C-terminus of a desired heavy or light chain of an amino acid. Further information can be found in U.S. Pat. No. 9,000,130, the contents of which are incorporated herein in its entirety. Derivatives of cysteine (Cys) include but are not limited to beta-2-Cys, beta-3-Cys, homocysteine, and N-methyl cysteine.

In some embodiments, the antibodies of the present disclosure include those having one or more engineered cysteine (eCys) residues. In some embodiments, derivatives of cysteine (Cys) include, but are not limited to beta-2-Cys, beta-3-Cys, homocysteine, and N-methyl cysteine.

An “antigen” is an entity to which an antibody specifically binds.

The terms “specific binding” and “specifically binds” mean that the antibody or antibody fragment thereof will bind, in a selective manner, with its corresponding target antigen and not with a multitude of other antigens. Typically, the antibody or antibody fragment binds with an affinity of at least about 1×10⁻⁷ M, for example, 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “amino acid” as used herein, refers to natural and non-natural, and proteogenic amino acids. Exemplary amino acids include, but are not limited to alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, ornithine, β-alanine, citrulline, serine methyl ether, aspartate methyl ester, glutamate methyl ester, homoserine methyl ether, and N,N-dimethyl lysine.

A “sugar moiety” as used herein, refers to a monovalent radical of monosaccharide, for example, a pyranose or a furanose. A sugar moiety may comprise a hemiacetal or a carboxylic acid (from oxidation of the pendant —CH₂OH group). In some embodiments, the sugar moiety is in the β-D conformation. In some embodiments, the sugar moiety is a glucose, glucuronic acid, or mannose group.

The term “inhibit” or “inhibition of” means to reduce by a measurable amount, or to prevent entirely (e.g., 100% inhibition).

The term “therapeutically effective amount” refers to an amount of an ADC as described herein that is effective to treat a disease or disorder in a mammal. In the case of cancer, the therapeutically effective amount of the ADC provides one or more of the following biological effects: reduction of the number of cancer cells; reduction of tumor size; inhibition of cancer cell infiltration into peripheral organs; inhibition of tumor metastasis; inhibition, to some extent, of tumor growth; and/or relief, to some extent, of one or more of the symptoms associated with the cancer. For cancer therapy, efficacy, in some aspects, is measured by assessing the time to disease progression (TTP) and/or determining the response rate (RR).

Unless otherwise indicated or implied by context, the term “substantial” or “substantially” refers to a majority, i.e. >50% of a population, of a mixture, or a sample, typically more than 50%, 55%, 60%, 65%, 70%, 75%, 80%, 85%, 90%, 91%, 92%, 93%, 94%, 95%, 96%, 97%, 98%, or 99%.

The terms “intracellularly cleaved” and “intracellular cleavage” refer to a metabolic process or reaction occurring inside a cell, in which the cellular machinery acts on the ADC or a fragment thereof, to intracellularly release free drug from the ADC, or other degradant products thereof. The moieties resulting from that metabolic process or reaction are thus intracellular metabolites.

The terms “cancer” and “cancerous” refer to or describe the physiological condition or disorder in mammals that is typically characterized by unregulated cell growth. A “tumor” comprises multiple cancerous cells.

“Subject” as used herein refers to an individual to which an ADC is administered. Examples of a “subject” include, but are not limited to, a mammal such as a human, rat, mouse, guinea pig, non-human primate, pig, goat, cow, horse, dog, cat, bird and fowl. Typically, a subject is a rat, mouse, dog, non-human primate, or human. In some aspects, the subject is a human.

The terms “treat” or “treatment,” unless otherwise indicated or implied by context, refer to therapeutic treatment and prophylactic measures to prevent relapse, wherein the object is to inhibit an undesired physiological change or disorder, such as, for example, the development or spread of cancer. For purposes of the present disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” in some aspects also means prolonging survival as compared to expected survival if not receiving treatment.

In the context of cancer, the term “treating” includes any or all of: inhibiting growth of cancer cells or of a tumor; inhibiting replication of cancer cells, lessening of overall tumor burden or decreasing the number of cancer cells, and ameliorating one or more symptoms associated with the disease.

The term “salt,” as used herein, refers to organic or inorganic salts of a compound, such as a Drug Unit (D), a linker such as those described herein, or an ADC. In some aspects, the compound contains at least one amino group, and accordingly acid addition salts can be formed with the amino group. Exemplary salts include, but are not limited to, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A salt may involve the inclusion of another molecule such as an acetate ion, a succinate ion, or other counterion. The counterion may be any organic or inorganic moiety that stabilizes the charge on the parent compound. Furthermore, a salt has one or more than one charged atom in its structure. In instances where there are multiple charged atoms as part of the salt, multiple counter ions can be present. Hence, a salt can have one or more charged atoms and/or one or more counterions. A “pharmaceutically acceptable salt” is one that is suitable for administration to a subject as described herein and in some aspects includes salts as described by P. H. Stahl and C. G. Wermuth, editors, Handbook of Pharmaceutical Salts: Properties, Selection and Use, Weinheim/Zürich:Wiley-VCH/VHCA, 2002, the list for which is specifically incorporated by reference in its entirety.

The term “tautomer,” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer.

The term “halo” or “halogen” refers to fluoro, chloro, bromo, or iodo.

The term “alkyl” refers to an unsubstituted straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) and is derived by the removal of one hydrogen atom from the parent alkane. Representative straight chain “C₁-C₈ alkyl” groups include, but are not limited to, methyl, ethyl, n-propyl, n-butyl, n-pentyl, n-hexyl, n-heptyl and n-octyl; while branched C₁-C₈ alkyls include, but are not limited to, isopropyl, sec-butyl, isobutyl, tert-butyl, isopentyl, and 2-methylbutyl.

The term “alkylene” refers to a bivalent unsubstituted saturated branched or straight chain hydrocarbon of the stated number of carbon atoms (e.g., a C₁-C₆ alkylene has from 1 to 6 carbon atoms) and having two monovalent radical centers derived by the removal of two hydrogen atoms from the same or two different carbon atoms of the parent alkane. Alkylene groups can be substituted with 1-6 fluoro groups, for example, on the carbon backbone (as —CHF— or —CF₂—) or on terminal carbons of straight chain or branched alkylenes (such as —CHF₂ or —CF₃). Alkylene radicals include but are not limited to: methylene (—CH₂—), ethylene (—CH₂CH₂—), n-propylene (—CH₂CH₂CH₂—), n-propylene (—CH₂CH₂CH₂—), n-butylene (—CH₂CH₂CH₂CH₂—), difluoromethylene (—CF₂—), tetrafluoroethylene (—CF₂CF₂—), and the like.

The term “alkenyl” refers to an unsubstituted straight chain or branched, hydrocarbon having at least one carbon-carbon double bond and the indicated number of carbon atoms (e.g., “C₂-C₈ alkenyl” or “C₂-C₁₀” alkenyl have from 2 to 8 or 2 to 10 carbon atoms, respectively). When the number of carbon atoms is not indicated, the alkenyl group has from 2 to 6 carbon atoms.

The term “alkynyl” refers to an unsubstituted straight chain or branched, hydrocarbon having at least one carbon-carbon triple bond and the indicated number of carbon atoms (e.g., “C₂-C₈ alkynyl” or “C₂-C₁₀” alkynyl have from 2 to 8 or 2 to 10 carbon atoms, respectively). When the number of carbon atoms is not indicated, the alkynyl group has from 2 to 6 carbon atoms.

The term “heteroalkyl” refers to a stable straight or branched chain saturated hydrocarbon having the stated number of total atoms and at least one (e.g., 1 to 15) heteroatom selected from the group consisting of O, N, Si and S. The carbon and heteroatoms of the heteroalkyl group can be oxidized (e.g., to form ketones, N-oxides, sulfones, and the like) and the nitrogen atoms can be quaternized. The heteroatom(s) can be placed at any interior position of the heteroalkyl group and/or at the position at which the heteroalkyl group is attached to the remainder of the molecule. Heteroalkyl groups can be substituted with 1-6 fluoro groups, for example, on the carbon backbone (as —CHF— or —CF₂—) or on terminal carbons of straight chain or branched heteroalkyls (such as —CHF₂ or —CF₃). Examples of heteroalkyl groups include, but are not limited to, —CH₂—CH₂—O—CH₃, —CH₂—CH₂—NH—CH₃, —CH₂—CH₂—N(CH₃)₂, —C(═O)—NH—CH₂—CH₂—NH—CH₃, —C(═O)—N(CH₃)—CH₂—CH₂—N(CH₃)₂, —C(═O)—NH—CH₂—CH₂—NH—C(═O)—CH₂—CH₃, —C(═O)—N(CH₃)—CH₂—CH₂—N(CH₃)—C(═O)—CH₂—CH₃, —O—CH₂—CH₂—CH₂—NH(CH₃), —O—CH₂—CH₂—CH₂—N(CH₃)₂, —O—CH₂—CH₂—CH₂—NH—C(═O)—CH₂—CH₃, —O—CH₂—CH₂—CH₂—N(CH₃)—C(═O)—CH₂—CH₃, —CH₂—CH₂—CH₂—NH(CH₃), —O—CH₂—CH₂—CH₂—N(CH₃)₂, —CH₂—CH₂—CH₂—NH—C(═O)—CH₂—CH₃, —CH₂—CH₂—CH₂—N(CH₃)—C(═O)—CH₂—CH₃, —CH₂—S—CH₂—CH₃, —CH₂—CH₂—S(O)—CH₃, —NH—CH₂—CH₂—NH—C(═O)—CH₂—CH₃, —CH₂—CH₂—S(O)₂—CH₃, —CH₂—CH₂—O—CF₃, and —Si(CH₃)₃. Up to two heteroatoms may be consecutive, such as, for example, —CH₂—NH—OCH₃ and —CH₂—O—Si(CH₃)₃. A terminal polyethylene glycol (PEG) moiety is a type of heteroalkyl group.

The term “heteroalkylene” refers to a bivalent unsubstituted straight or branched group derived from heteroalkyl (as defined herein). Examples of heteroalkylene groups include, but are not limited to, —CH₂—CH₂—O—CH₂—, —CH₂—CH₂—O—CF₂—, —CH₂—CH₂—NH—CH₂—, —C(═O)—NH—CH₂—CH₂—NH—CH₂— —C(═O)—N(CH₃)—CH₂—CH₂—N(CH₃)—CH₂—, —C(═O)—NH—CH₂—CH₂—NH—C(═O)—CH₂—CH₂—, —C(═O)—N(CH₃)—CH₂—CH₂—N(CH₃)—C(═O)—CH₂—CH₂—, —O—CH₂—CH₂—CH₂—NH—CH₂—, —O—CH₂—CH₂—CH₂—N(CH₃)—CH₂—, —O—CH₂—CH₂—CH₂—NH—C(═O)—CH₂—CH₂—, —O—CH₂—CH₂—CH₂—N(CH₃)—C(═O)—CH₂—CH₂—, —CH₂—CH₂—CH₂—NH—CH₂—, —CH₂—CH₂—CH₂—N(CH₃)—CH₂—, —CH₂—CH₂—CH₂—NH—C(═O)—CH₂—CH₂—, —CH₂—CH₂—CH₂—N(CH₃)—C(═O)—CH₂—CH₂—, —CH₂—CH₂—NH—C(═O)—, —CH₂—CH₂—N(CH₃)—CH₂—, —CH₂—CH₂—N⁺(CH₃)₂—, —NH—CH₂—CH₂(NH₂)—CH₂—, and —NH—CH₂—CH₂(NHCH₃)—CH₂—. A bivalent polyethylene glycol (PEG) moiety is a type of heteroalkylene group.

The term “alkoxy” refers to an alkyl group, as defined herein, which is attached to a molecule via an oxygen atom. For example, alkoxy groups include, but are not limited to methoxy, ethoxy, n-propoxy, iso-propoxy, n-butoxy, sec-butoxy, tert-butoxy, n-pentoxy and n-hexoxy.

The term “alkylthio” refers to an alkyl group, as defined herein, which is attached to a molecule via a sulfur atom. For example, alkythio groups include, but are not limited to thiomethyl, thioethyl, thio-n-propyl, thio-iso-propyl, and the like.

The term “haloalkyl” refers to an unsubstituted straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) wherein at least one hydrogen atom of the alkyl group is replaced by a halogen (e.g., fluoro, chloro, bromo, or iodo). When the number of carbon atoms is not indicated, the haloalkyl group has from 1 to 6 carbon atoms. Representative C₁₋₆ haloalkyl groups include, but are not limited to, trifluoromethyl, 2,2,2-trifluoroethyl, and 1-chloroisopropyl.

The term “haloalkoxy” refers to a haloalkyl group, as defined herein, which is attached to a molecule via an oxygen atom. For example, haloalkoxy groups include, but are not limited to trifluoromethoxy, 2,2,2-trifluoroethoxy, and 1,1,1-trifluoro2-methylpropoxy.

The term “cycloalkyl” refers to a cyclic, saturated or partially unsaturated hydrocarbon having the indicated number of carbon atoms (e.g., “C₃₋₈ cycloalkyl” or “C₃₋₆” cycloalkyl have from 3 to 8 or 3 to 6 carbon atoms, respectively). When the number of carbon atoms is not indicated, the cycloalkyl group has from 3 to 6 carbon atoms. Cycloalkyl groups include bridged, fused, and spiro ring systems, and bridged bicyclic systems where one ring is aromatic and the other is unsaturated. Representative “C3.6 cycloalkyl” groups include, cyclopropyl, cyclobutyl, cyclopentyl, and cyclohexyl.

The term “aryl” refers to an unsubstituted monovalent carbocyclic aromatic hydrocarbon radical of 6-10 carbon atoms derived by the removal of one hydrogen atom from a single carbon atom of a parent aromatic ring system. Aryl groups include, but are not limited to, phenyl, naphthyl, anthracenyl, biphenyl, and the like.

The term “heterocycle” refers to a saturated or partially unsaturated ring or a multiple condensed ring system, including bridged, fused, and spiro ring systems. Heterocycles can be described by the total number of atoms in the ring system, for example a 3-10 membered heterocycle has 3 to 10 total ring atoms. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more heterocycles (e.g., decahydronapthyridinyl), carbocycles (e.g., decahydroquinolyl) or aryls. The rings of a multiple condensed ring system can be connected to each other via fused, spiro and bridged bonds when allowed by valency requirements. It is to be understood that the point of attachment of a multiple condensed ring system (as defined above for a heterocycle) can be at any position of the multiple condensed ring system including a heterocycle, aryl and carbocycle portion of the ring. It is also to be understood that the point of attachment for a heterocycle or heterocycle multiple condensed ring system can be at any suitable atom of the heterocycle or heterocycle multiple condensed ring system including a carbon atom and a heteroatom (e.g., a nitrogen). Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, and 1,4-benzodioxanyl.

The term “heteroaryl” refers to an aromatic hydrocarbon ring system with at least one heteroatom within a single ring or within a fused ring system, selected from the group consisting of O, N and S. The ring or ring system has 4n+2 electrons in a conjugated a system where all atoms contributing to the conjugated π system are in the same plane. In some embodiments, heteroaryl groups have 5-10 total ring atoms and 1, 2, or 3 heteroatoms (referred to as a “5-10 membered heteroaryl”). Heteroaryl groups include, but are not limited to, imidazole, triazole, thiophene, furan, pyrrole, benzimidazole, pyrazole, pyrazine, pyridine, pyrimidine, and indole.

The term “hydroxyl” refers to an —OH radical.

The term “cyano” refers to a —CN radical.

The term “carboxy” refers to a —C(═O)OH radical.

The term “oxo” refers to a ═O radical.

The term “succinimide” as used as part of an antibody-drug conjugate (ADC) refers to:

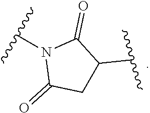 [SMILES: *C1CC(=O)N(*)C1=O]

The term “hydrolyzed succinimide” as used as part of an antibody-drug conjugate (ADC) refers to:

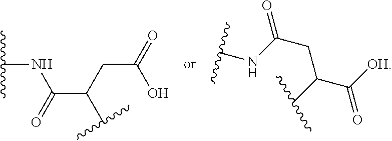 [SMILES: *NC(=O)C(*)CC(=O)O ; *NC(=O)CC(*)C(=O)O]

It will be appreciated by those skilled in the art that compounds of this disclosure having a chiral center may exist in and be isolated in optically active and racemic forms.

As used herein, the term “free drug” refers to a biologically active species that is not covalently attached to an antibody. Accordingly, free drug refers to any unconjugated compound, including a compound as it exists immediately upon cleavage from the ADC. The release mechanism can be via a cleavable linker in the ADC, or via intracellular conversion or metabolism of the ADC. In some aspects, the free drug will be protonated and/or may exist as a charged moiety. The free drug is a pharmacologically active species which is capable of exerting the desired biological effect. In some embodiments, the pharamacologically active species is the parent drug alone. In some embodiments, the pharamacologically active species is the parent drug bonded to a component or vestige of the ADC (e.g., a component of the linker, succinimide, hydrolyzed succinimide, and/or antibody that has not undergone subsequent intracellular metabolism). In some embodiments, free drug refers to a compound of Formula (I), as described herein, for example, wherein one or more of X^{B}, Y, W, A, and M¹ are absent. In some embodiments, free drug refers to a compound of Formula (II), as described herein. In some embodiments, free drug refers to a compound of Formula (II-A), as described herein. In some embodiments, free drug refers to a compound of Formula (III), as described herein. In some embodiments, free drug refers to a compound of Formula (IV), as described herein. In some embodiments, free drug refers to a compound of Formula (V), as described herein.

As used herein, the term “Drug Unit” refers to the free drug that is conjugated to an antibody in an ADC, as described herein.

### Antibody-Drug Conjugate (ADC) Compounds

Some embodiments provide an antibody-drug conjugate (ADC) comprising:
an antibody;
a linker, as described herein; and
a compound of Formula (I) as described herein;
wherein the compound of Formula (I) is conjugated to the linker; and
wherein each linker is conjugated to the antibody via a succinimide or hydrolyzed succinimide covalently linked to a sulfur atom of a cysteine residue.

Some embodiments provide an antibody-drug conjugate (ADC) having the formula:

Ab-(S*-M¹-(D))ₚ

wherein:
Ab is an antibody;
each S* is a sulfur atom from a cysteine residue of the antibody;
M¹ is a succinimide or a hydrolyzed succinimide;
subscript p is an integer from 2 to 8; and
each (D) is a Drug Unit of Formula (I):

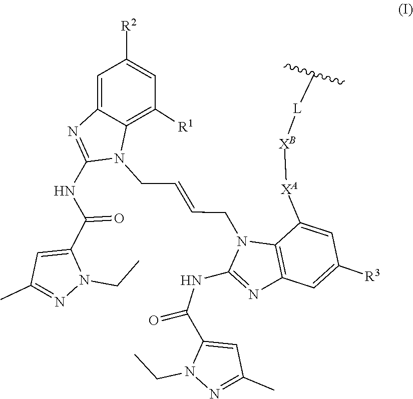

wherein:
[symbol] represents covalent attachment of L to M¹;
R¹ is hydrogen, hydroxyl, C₁₋₆ alkoxy, —(C₁₋₆ alkyl) C₁₋₆ alkoxy, —(CH₂)ₙ—NR^{A}R^{B}, or PEG2 to PEG4;
each R² and R³ are independently —CO₂H, —(C═O)ₘ—NR^{C}R^{D}, or —(CH₂)_{q}—NR^{E}R^{F};
each R^{A}, R^{B}, R^{C}, R^{D}, R^{E}, and R^{F} are independently hydrogen or C₁₋₃ alkyl;
each subscript n is independently an integer from 0 to 6;
each subscript m is independently 0 or 1;
each subscript q is an integer from 0 to 6;
X^{A} is —CH₂—, —O—, —S—, —NH—, or —N(CH₃)—;
X^{B} is absent or a 2-16 membered heteroalkylene;
X^{B}, M¹, and L are each independently optionally substituted with a PEG Unit from PEG1 to PEG72; and
L is an optional linker as described herein. When present, L is linked via a covalent bond to X^{B}, or X^{A} if X^{B} is absent, as depicted in Formula (I). When L is absent, M¹ is linked via a covalent bond to X^{B}, or X^{A} if X^{B} is absent, as depicted in Formula (I).

In some embodiments, M¹ is a succinimide. In some embodiments, M¹ is a hydrolyzed succinimide. It will be understood that a hydrolyzed succinimide may exist in two regioisomeric form(s). Those forms are exemplified below for hydrolysis of M¹ bonded to *S-Ab, wherein the structures representing the regioisomers from that hydrolysis are formula M¹a and M¹b; wherein the wavy lines adjacent to the bonds represent the covalent attachment to Formula (I).

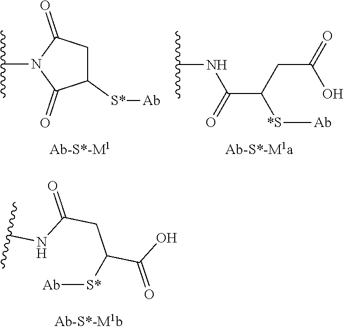

The M or M¹ groups, when present, are capable of linking an antibody to an A group, when present (or a W, Y, or X^{B} group if subscript a and/or subscript w and/or subscript y are 0). In this regard an antibody has a functional group that can form a bond with a functional group of M or M¹. Useful functional groups that can be present on an antibody, either naturally or via chemical manipulation include, but are not limited to, sulfhydryl (—SH), amino, hydroxyl, carboxy, and the anomeric hydroxyl group of a carbohydrate. In one aspect, the antibody functional groups are sulfhydryl and amino. Sulfhydryl groups can be generated by reduction of an intramolecular disulfide bond of an antibody. Alternatively, sulfhydryl groups can be generated by reaction of an amino group of a lysine moiety of an antibody using 2-iminothiolane (Traut's reagent) or another sulfhydryl generating reagent. In some embodiments, M or M¹ forms a bond with a sulfur atom of the antibody. The sulfur atom can be derived from a sulfhydryl group of the antibody.

In some embodiments, L has the formula -(A)ₐ-(W)_{w}-(Y)_{y}—, wherein:
A is a C₂₋₂₀ alkylene optionally substituted with 1-3 R^{a1}; or a 2 to 40 membered heteroalkylene optionally substituted with 1-3 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —C(O)NR^{d1}R^{e1}, —C(O)(C₁₋₆ alkyl), and —C(O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —C(O)NR^{d1}R^{e1}, —C(O)(C₁₋₆ alkyl), and —C(O)O(C₁₋₆ alkyl);
each R^{d1} and R^{e1} are independently hydrogen or C₁₋₃ alkyl;
W is from 1-12 amino acids or has the structure:

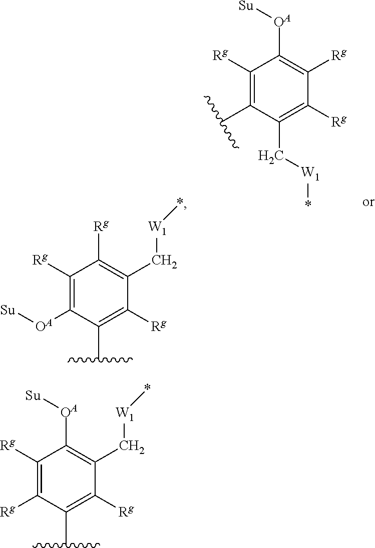

wherein Su is a Sugar moiety;
—O^{A}— represents a glycosidic bond;
each R^{g} is independently hydrogen, halogen, —CN, or —NO₂;
W₁ is absent or —O—C(═O)—;
[symbol] represents covalent attachment to A or M¹;
* represents covalent attachment to Y, X^{A}, or X^{B} in Formula (I);
Y is a self-immolative moiety, a non-self-immolative releasable moiety, or a non-cleavable moiety;
subscript a is 0 or 1;
subscript y is 0 or 1; and
subscript w is 0 or 1.

In some embodiments, R¹ is hydrogen. In some embodiments, R¹ is hydroxyl. In some embodiments, R¹ is C₁₋₆ alkoxy. In some embodiments, R¹ is methoxy. In some embodiments, R¹ is —(C₁₋₆ alkyl)C₁₋₆ alkoxy. In some embodiments, R¹ is methoxyethyl. In some embodiments, R¹ is PEG2 to PEG4.

In some embodiments, R¹ is —(CH₂)ₙ—NR^{A}R^{B}. In some embodiments, R^{A} and R^{B} are both hydrogen. In some embodiments, R^{A} and R^{B} are independently C₁₋₃ alkyl. In some embodiments, one of R^{A} and R^{B} is hydrogen and the other of R^{A} and R^{B} is C₁₋₃ alkyl. In some embodiments, the C₁₋₃ alkyl is methyl. In some embodiments, each subscript n is 0. In some embodiments, each subscript n is 1. In some embodiments, each subscript n is 2. In some embodiments, each subscript n is 3, 4, 5, or 6.

In some embodiments, each R² and R³ are independently —CO₂H, —(C═O)ₘ—N^{C}R^{D}, or —(CH₂)_{q}—NR^{E}R^{F}; and R² and R³ are the same. In some embodiments, each R² and R³ are independently —CO₂H, —(C═O)ₘ—NR^{C}R^{D}, or —(CH₂)_{q}—NR^{E}R^{F}; and R² and R³ are different.

In some embodiments, R² is —(C═O)ₘ—NR^{C}R^{D}. In some embodiments, R³ is —(C═O)ₘ—NR^{C}R^{D}. In some embodiments, R^{C} and R^{D} are both hydrogen. In some embodiments, R^{C} and R^{D} are each independently C₁₋₃ alkyl. In some embodiments, the C₁₋₃ alkyl is methyl. In some embodiments, one of R^{C} and R^{D} is hydrogen and the other of R^{C} and R^{D} is C₁₋₃ alkyl. In some embodiments, each subscript m is 0. In some embodiments, each subscript m is 1.

In some embodiments, R² is —(CH₂)_{q}—NR^{E}R^{F}. In some embodiments, R³ is —(CH₂)_{q}—NR^{E}R^{F}. In some embodiments, R^{E} and R^{F} are both hydrogen. In some embodiments, R^{E} and R^{F} are each independently C₁₋₃ alkyl. In some embodiments, the C₁₋₃ alkyl is methyl. In some embodiments, one of R^{E} and R^{F} is hydrogen and the other of R^{E} and R^{F} is C₁₋₃ alkyl. In some embodiments, each subscript q is 0. In some embodiments, each subscript q is an integer from 1 to 6. In some embodiments, each subscript q is 1. In some embodiments, each subscript q is 2. In some embodiments, each subscript q is 3, 4, 5, or 6.

In some embodiments, R³ is —CO₂H. In some embodiments, R² is —CO₂H.

In some embodiments, X^{A} is —CH₂—. In some embodiments, X^{A} is —O—. In some embodiments, X^{A} is —S—. In some embodiments, X^{A} is —NH—. In some embodiments, X^{A} is —N(CH₃)—.

In some embodiments, X^{B} is a 2-16 membered heteroalkylene. In some embodiments, X^{B} is a 2-12 membered heteroalkylene. In some embodiments, X^{B} is a 2-10 membered heteroalkylene. In some embodiments, X^{B} is a 2-8 membered heteroalkylene. In some embodiments, X^{B} is a 4-8 membered heteroalkylene. In some embodiments, the heteroalkylene is straight chained. In some embodiments, the heteroalkylene is branched. In some embodiments, the heteroalkylene is branched, having 1-4 methyl groups. In some embodiments, the heteroalkylene is branched, having 1 or 2 methyl groups. In some embodiments, the heteroalkylene is substituted with 1-3 fluoro groups. In some embodiments, X^{B} comprises one or two nitrogen atoms. In some embodiments, X^{B} comprises one or two oxo groups. In some embodiments, X^{B} comprises one nitrogen atom and one oxo group. In some embodiments, X^{B} comprises two nitrogen atoms and two oxo groups. In some embodiments, X^{B} comprises a carbamate.

In some embodiments, the covalent attachment of Y and X^{B} comprises an amide. In some embodiments, the covalent attachment of Y and X^{B} comprises a carbamate. In some embodiments, the covalent attachment of Y and X^{B} comprises an ether.

In some embodiments, X^{B} is

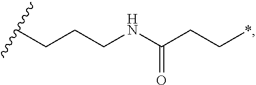

wherein [symbol] represents covalent attachment to X^{A}, and * represents covalent attachment to L, when present, or M¹. In some embodiments, X^{B} is

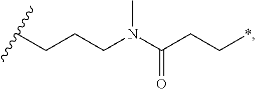

wherein [symbol] represents covalent attachment to X^{A}, and * represents covalent attachment to L, when present, or M¹. In some embodiments, X^{B} is

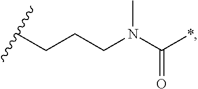

wherein [symbol] represents covalent attachment to X^{A}, and * represents covalent attachment to L, when present, or M¹. In some embodiments, X^{B} is

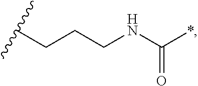

wherein [symbol] represents covalent attachment to X^{A}, and * represents covalent attachment to L, when present, or M¹. In some embodiments, X^{B} is

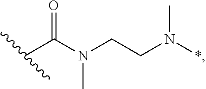

wherein [symbol] covalent attachment to X^{A}, and * represents covalent attachment to L, when present, or M¹. In some embodiments, X^{B} is

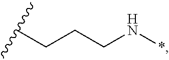

wherein [symbol] represents covalent attachment to X^{A}, and * represents covalent attachment to L, when present, or M¹.

In some embodiments, X^{B} is selected from the group consisting of the structures below, wherein [symbol] represents covalent attachment to X^{A}, and * represents covalent attachment to L, when present, or M¹.

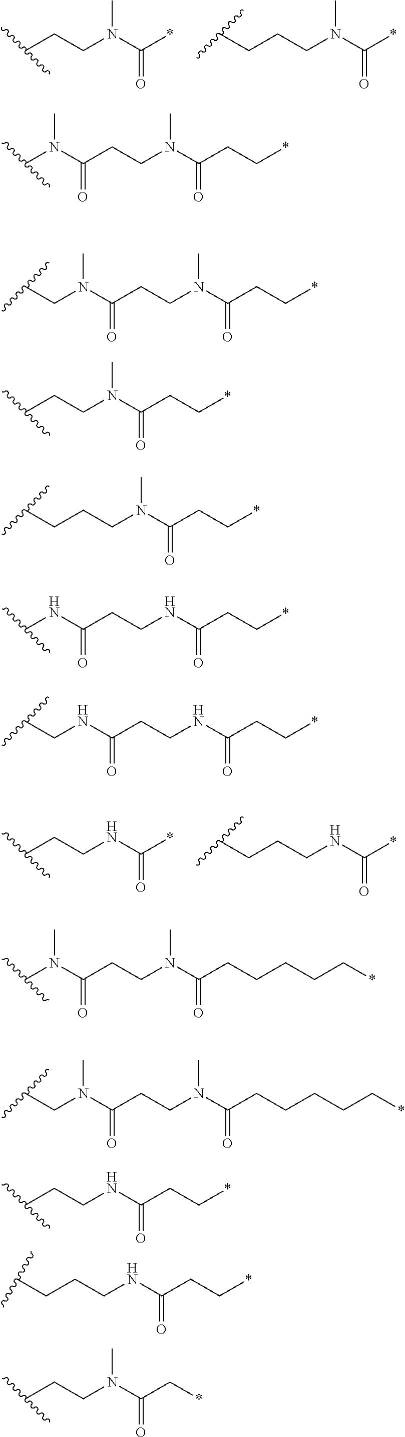

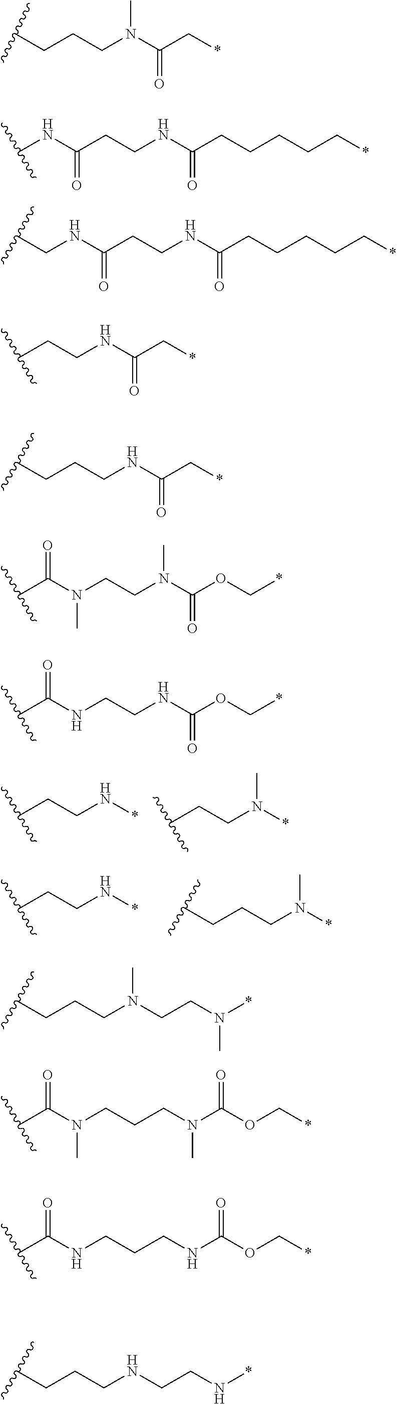

In some embodiments, one of X^{B} and L is substituted with a PEG Unit from PEG1 to PEG72, as described herein. In some embodiments, X^{B} and L are each substituted with an independently selected PEG Unit from PEG2 to PEG72, as described herein. In some embodiments, each PEG Unit from PEG 1 to PEG72 can range from PEG8 to PEG12, PEG12 to PEG24, or PEG36 to PEG72. In some embodiments, each PEG Unit from PEG 1 to PEG72 is PEG8 to PEG24.

In some embodiments, X^{B} and L are unsubstituted.

In some embodiments, R¹ is methoxy; R² and R³ are both —C(═O)NH₂; and X^{A} is —O—.

In some embodiments, L is absent and X^{A}—X^{B}-M¹ is selected from the group consisting of:

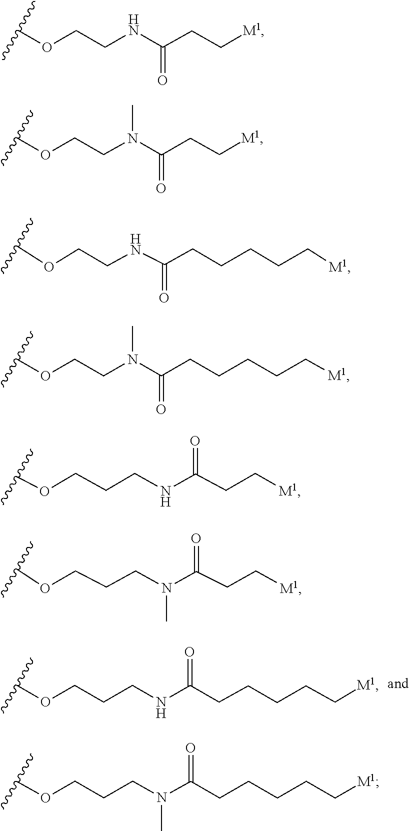

wherein [symbol] represents covalent attachment to the remainder of Formula (I).

In some embodiments, X^{A}—X^{B}-L is selected from:

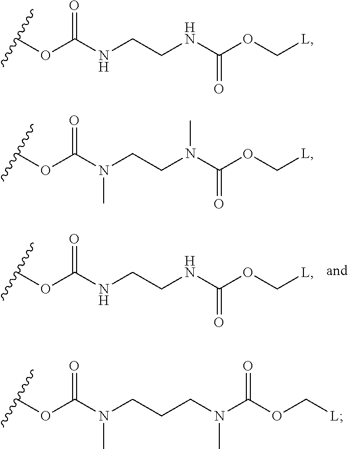

wherein [symbol] represents covalent attachment to the remainder of Formula (I).

In some embodiments, R¹ is methoxy and R² and R³ are both —C(═O)NH₂. In some embodiments, X^{A} is —O— and X^{B} is

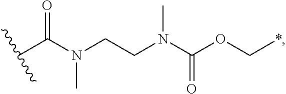

wherein [symbol] represents covalent attachment to X^{A} and * represents covalent attachment to L, when present, or M¹. In some embodiments, R¹ is methoxy; R² and R³ are both —C(═O)NH₂; X^{A} is —O—; and X^{B} is

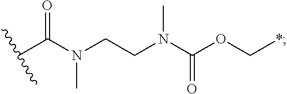

wherein [symbol] represents covalent attachment to X^{A} and * represents covalent attachment to L, when present, or M¹. In some embodiments, R¹ is methoxy; R² and R³ are both —C(═O)NH₂; X^{A} is —O—; X^{B} is

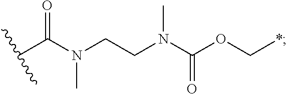

[symbol]

represents covalent attachment to X^{A} and * represents covalent attachment to L; and subscript a and subscript y are both 0.

In some embodiments, X^{B} is absent.

In some embodiments, subscript p is an integer from 2 to 8, from 2 to 6, from 2 to 4, from 4 to 8, or from 6 to 8. In some embodiments, subscript p is 2, 4, 6, or 8. In some embodiments, subscript p is 2. In some embodiments, subscript p is 4. In some embodiments, subscript p is 6. In some embodiments, subscript p is 8.

In some embodiments, X^{B} is absent and L is covalently attached to X^{A}. In some embodiments, X^{B} is absent and Y is covalently attached to X^{A}. In some embodiments, X^{B} is absent and Y is absent, and W is covalently attached to X^{A}. In some embodiments, X^{B} is absent, Y is absent, W is absent, and A is covalently attached to X^{A}.

In some embodiments, X^{B} is 2-16 membered heteroalkylene and L is covalently attached to X^{B}. In some embodiments, X^{B} is 2-16 membered heteroalkylene and Y is covalently attached to X^{B}. In some embodiments, X^{B} is 2-16 membered heteroalkylene, Y is absent, and W is covalently attached to X^{B}. In some embodiments, X^{B} is 2-16 membered heteroalkylene, Y is absent, W is absent, and A is covalently attached to X^{B}.

In some embodiments, W₁ is —OC(═O)— and subscript y is 1. In some embodiments, X^{A} is —O— and X^{B} and W₁ are absent. In some embodiments, X^{A} is NH or —O—, X^{B} is absent, and W₁ is —OC(═O). In some embodiments, X^{A} is —N(CH₃)—, X^{B} is absent, and W₁ is —OC(═O). In some embodiments, X^{A} is —S—, X^{B} is absent, and W₁ is —OC(═O). In some embodiments, W₁ is —OC(═O)— and X^{B} is covalently attached to W via —O— or —NH—.

In some embodiments, A is covalently attached to M¹. In some embodiments, when subscript a is 0, W is covalently attached to M¹. In some embodiments, when subscript a is 0 and subscript w is 0, Y is covalently attached to M¹. In some embodiments, when subscripts a, y, and w, are each 0, X^{B} is covalently attached to M¹.

In some embodiments, the ADC has the formula:

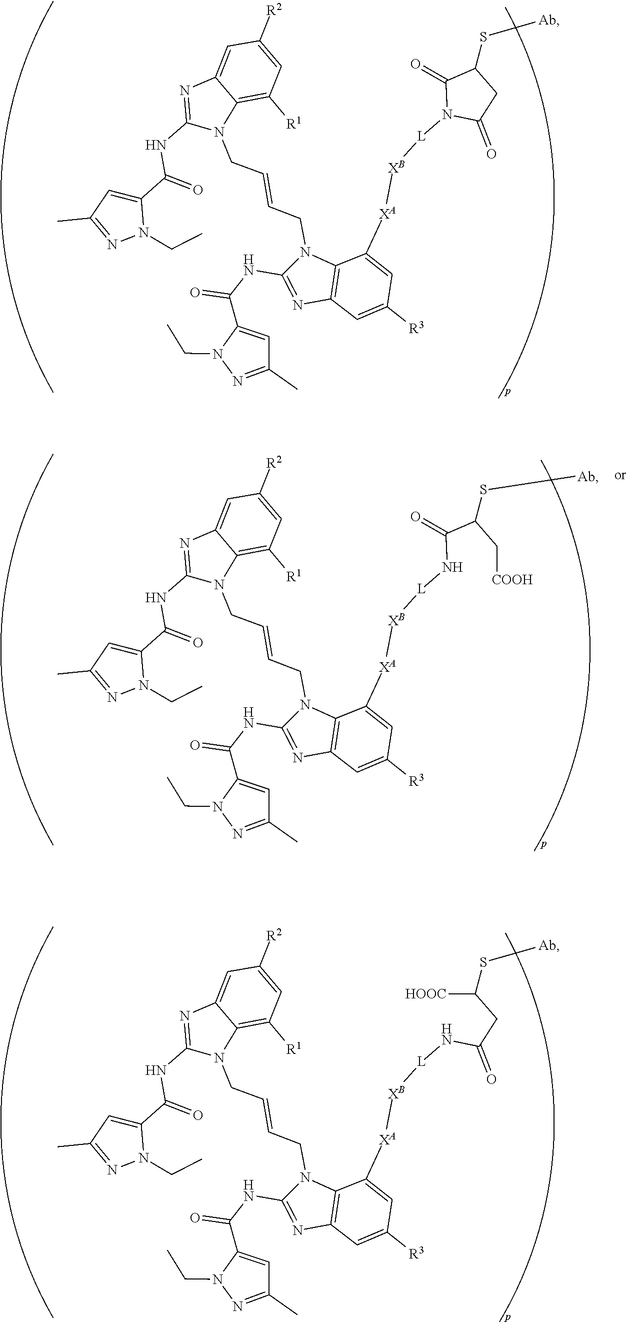

wherein:
Ab is an antibody;
R¹, R², R³, X^{A}, X^{B}, and L are as defined above in connection with Formula (I); and
each subscript p is independently an integer from 2 to 8.

In some aspects, the ADC has the formula:

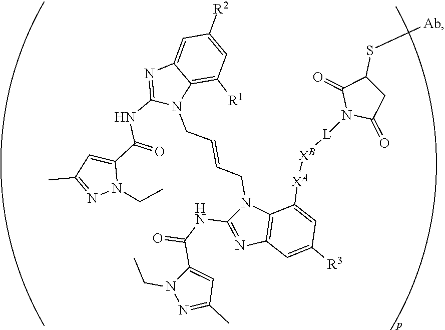

wherein:
Ab is an antibody;
R¹, R², R³, X^{A}, X^{B}, and L are as defined above in connection with Formula (I); and
each subscript p is independently an integer from 2 to 8.

In some aspects, the ADC has the formula:

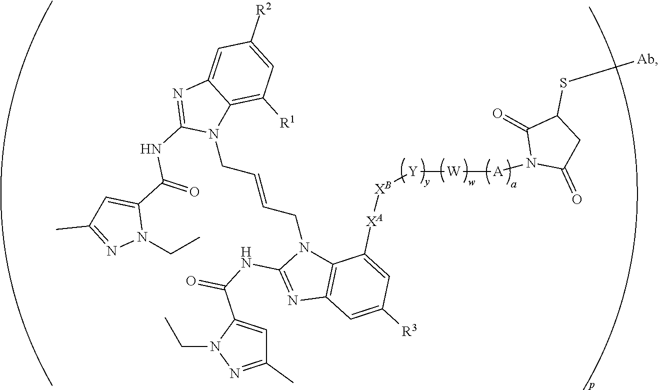

wherein:
Ab is an antibody;
R¹, R², R³, X^{A}, X^{B}, Y, W, and A are as defined above in connection with Formula (I);
each subscript y is independently 0 or 1;
each subscript w is independently 0 or 1;
each subscript a is independently 0 or 1; and
each subscript p is independently an integer from 2 to 8.

In some embodiments, the ADC has the formula:

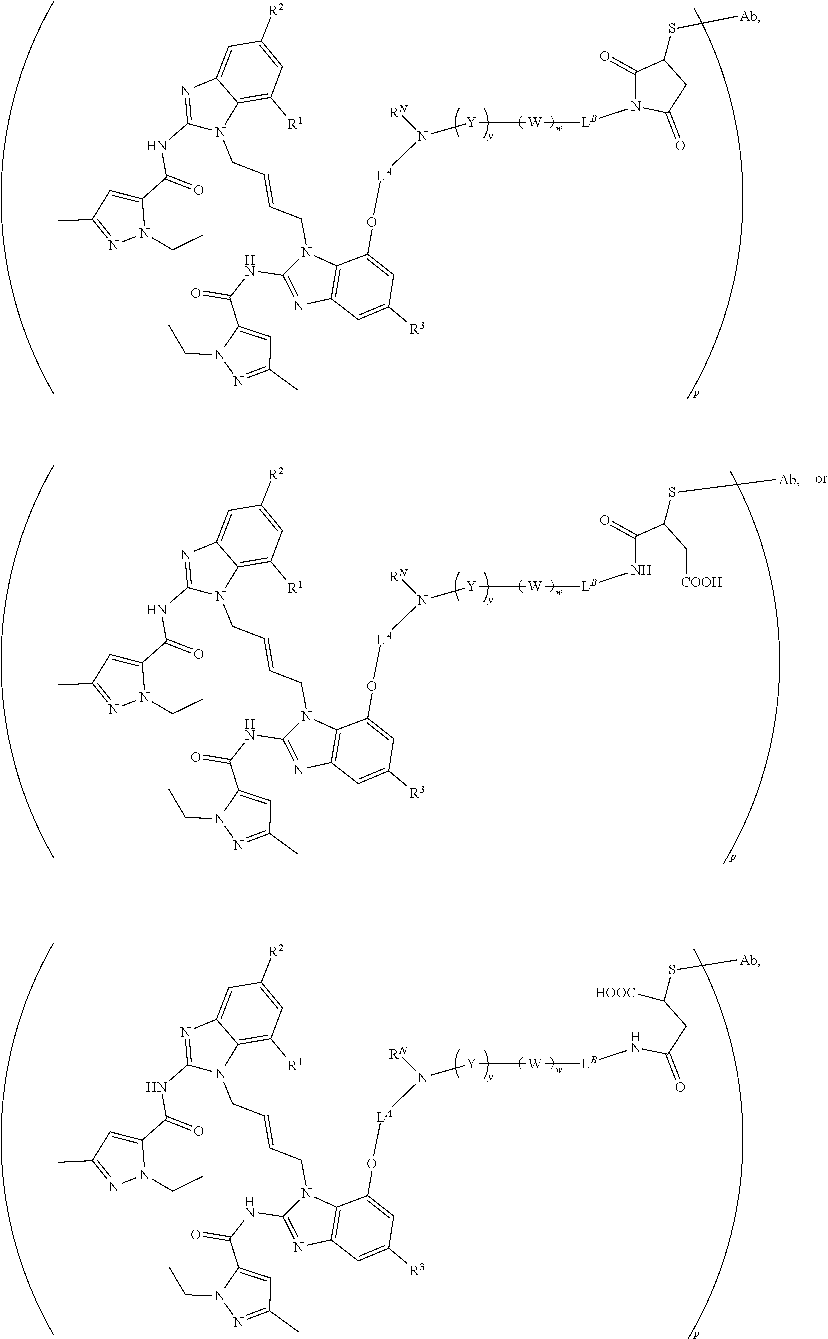

wherein:
Ab is an antibody;
R¹, R², R³, L^{A}, R^{N}, Y, W, and L^{B} are as defined below in connection with Formula (II-A);
each subscript y is independently 0 or 1;
each subscript w is independently 0 or 1; and
each subscript p is independently an integer from 2 to 8.

In some aspects, the ADC has the formula:

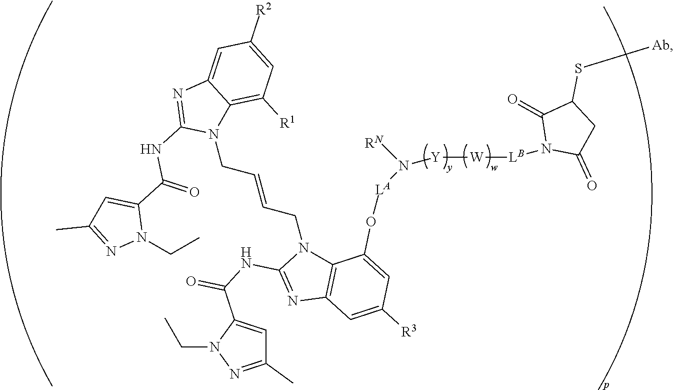

wherein:
Ab is an antibody;
R¹, R², R³, L^{A}, R^{N}, Y, W, and L^{B} are as defined below in connection with Formula (II-A);
each subscript y is independently 0 or 1;
each subscript w is independently 0 or 1; and
each subscript p is independently an integer from 2 to 8.

In some embodiments, the ADC has the formula:

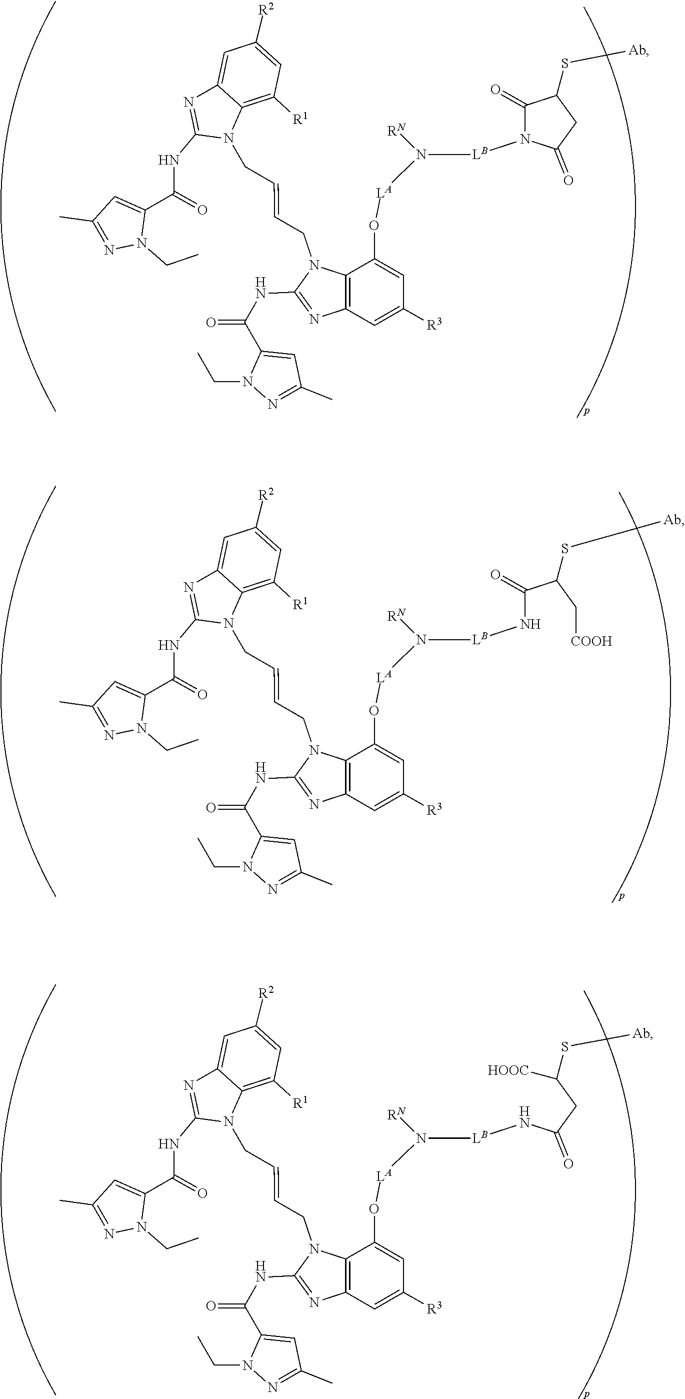

wherein:
Ab is an antibody;
R¹, R², R³, L^{A}, R^{N}, and L^{B} are as defined below in connection with Formula (II-B); and
each subscript p is independently an integer from 2 to 8.

In some aspects, the ADC has the formula:

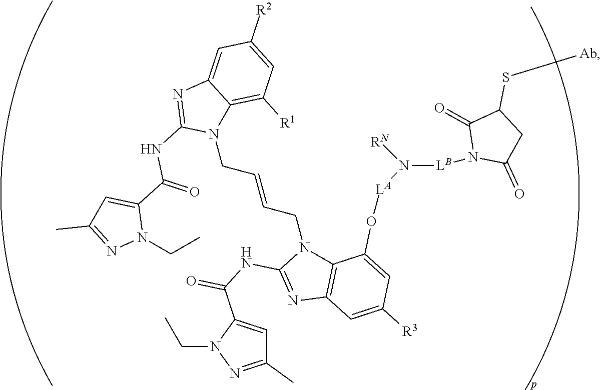

wherein:
Ab is an antibody;
R¹, R², R³, L^{A}, R^{N}, and L^{B} are as defined below in connection with Formula (II-B); and each subscript p is independently an integer from 2 to 8.

Some embodiments provide an antibody-drug conjugate (ADC) having the formula:

Ab-(S*-(D′))ₚ

wherein:
Ab is an antibody;
each S* is a sulfur atom from a cysteine residue of the antibody;
D′ is a drug unit that is a radical of the compound of Formula (IV), as described below; and
subscript p is an integer from 2 to 8.

In some embodiments, the radical of the compound of Formula (IV) comprises a radical in substituent M within Formula (IV). In some embodiments, the drug unit D′ has the structure:

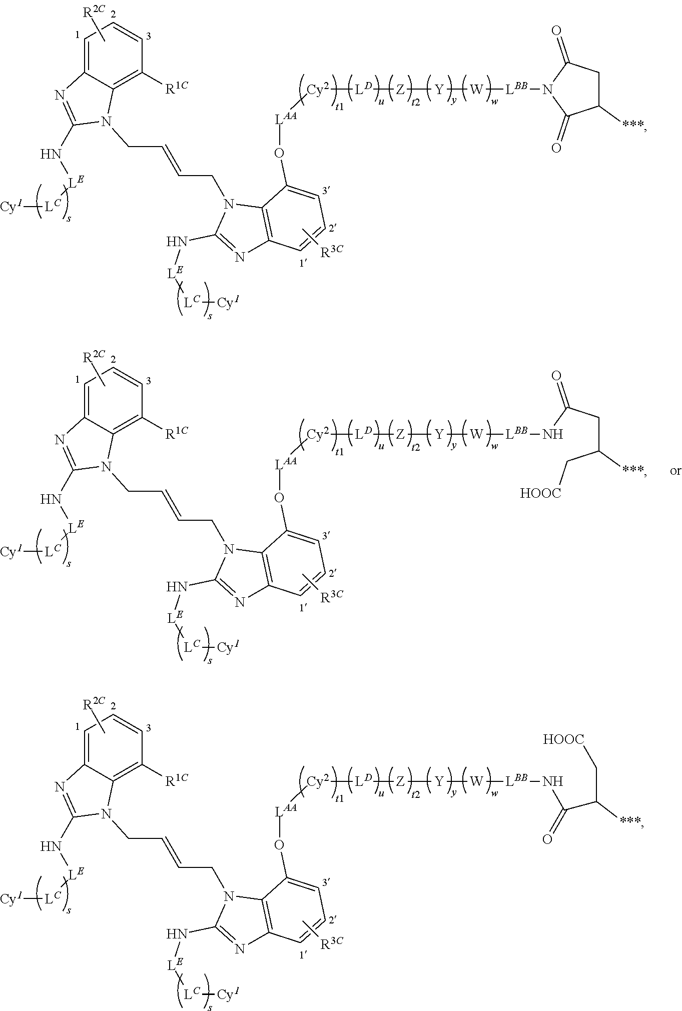

where *** indicates attachment to S* and the remaining variables are as defined below in connection with Formula (IV).

In some aspects, the drug unit D′ has the structure:

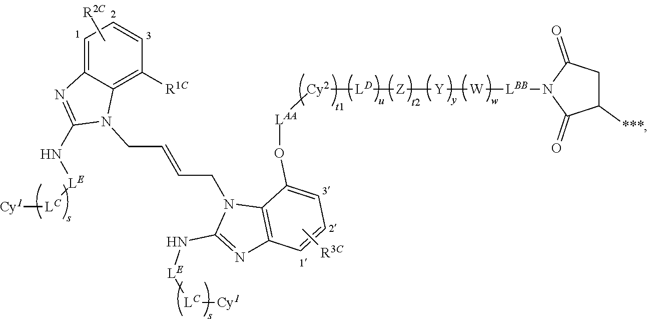

where *** indicates attachment to S* and the remaining variables are as defined below in connection with Formula (IV).

In some embodiments, the ADC has the formula:

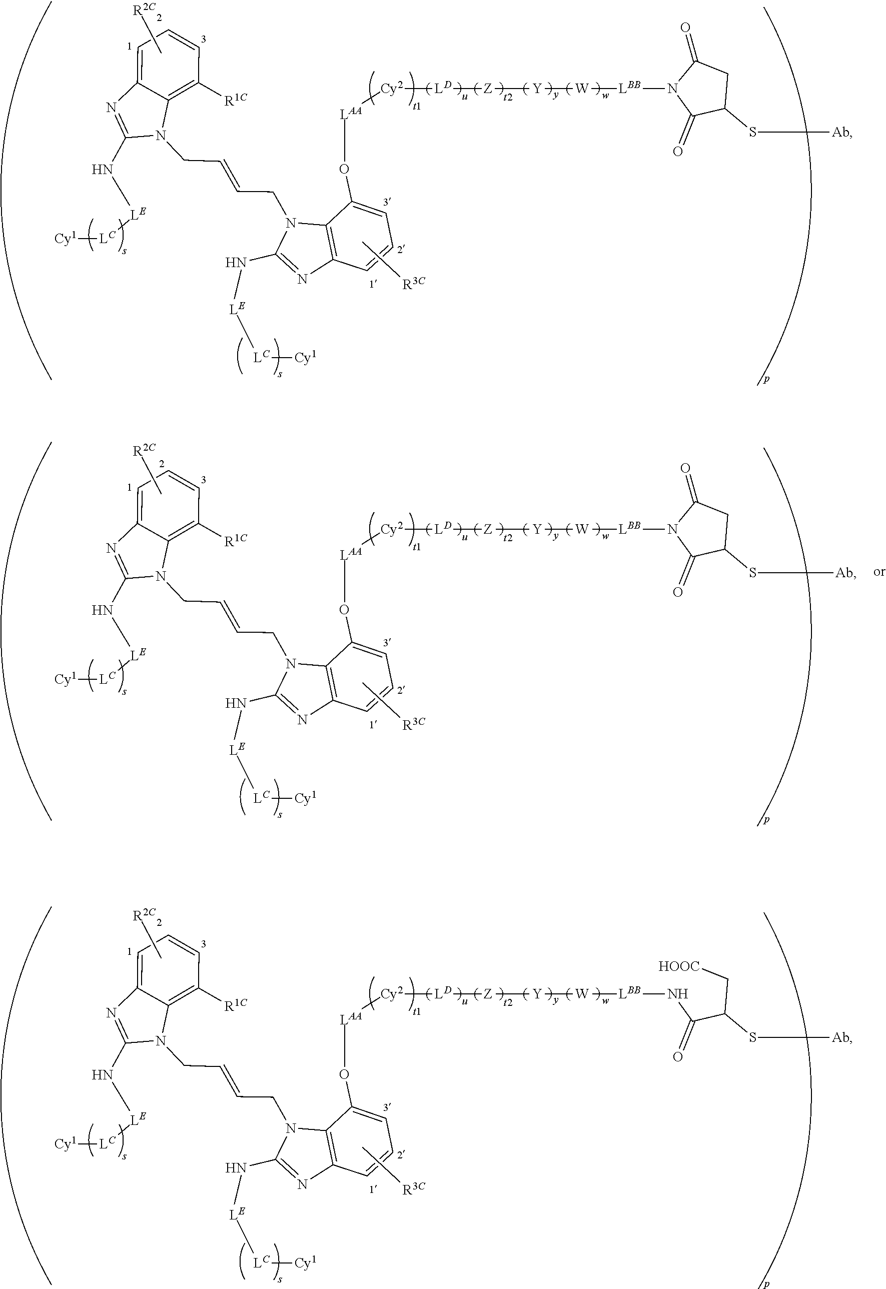

wherein:
Ab is an antibody;
each subscript p is independently an integer from 2 to 8; and
the remaining variables are as defined below in connection with Formula (IV).

In some aspects, the ADC has the formula:

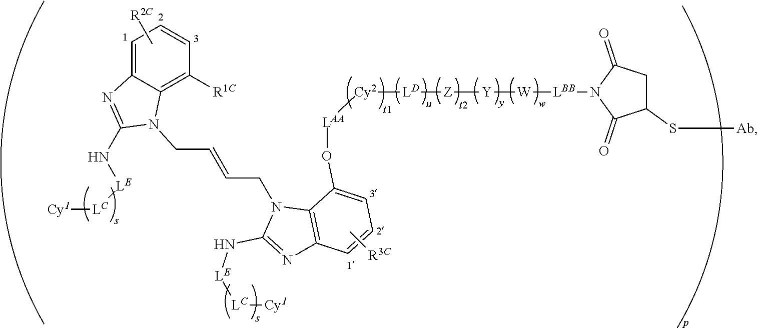

wherein:
Ab is an antibody;
each subscript p is independently an integer from 2 to 8; and
the remaining variables are as defined below in connection with Formula (IV).

Some embodiments provide an antibody-drug conjugate (ADC) selected from the group consisting of:

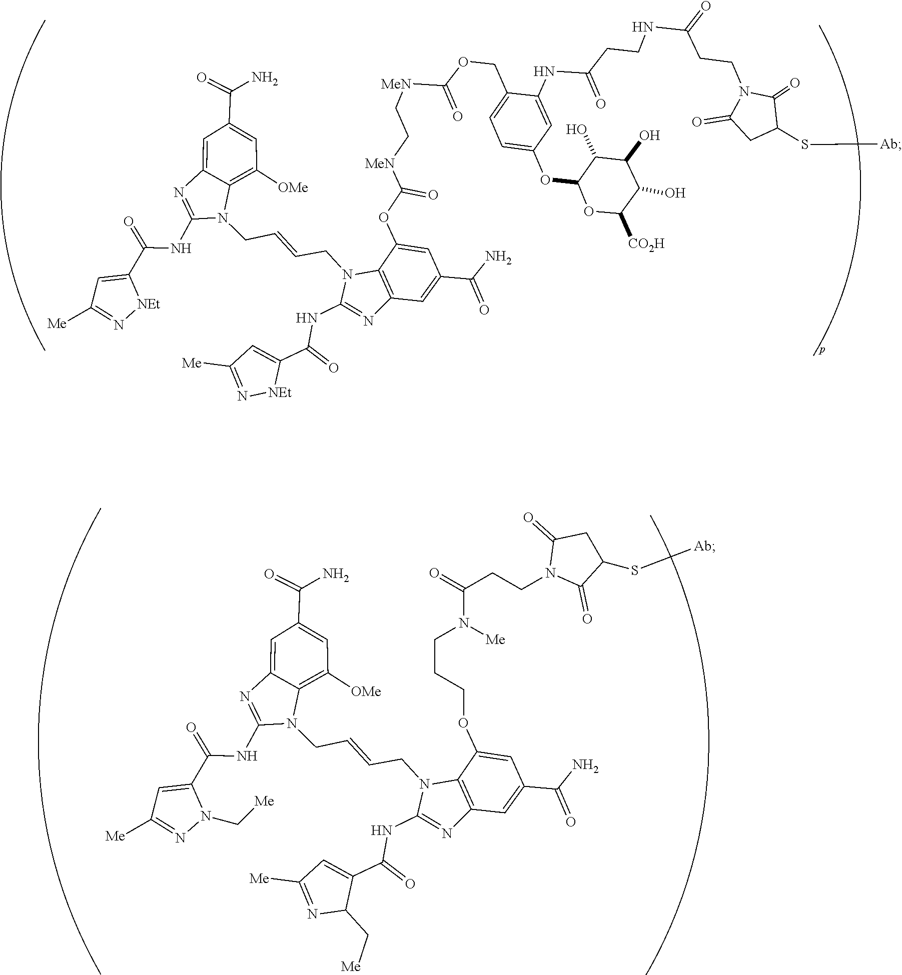

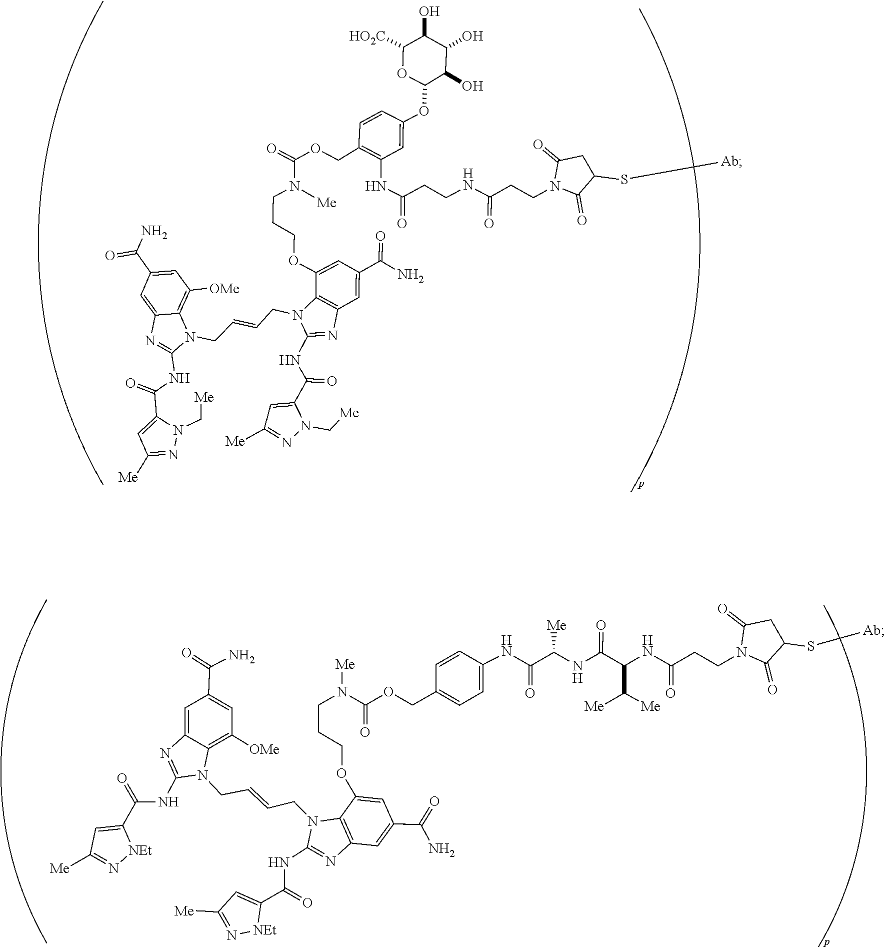

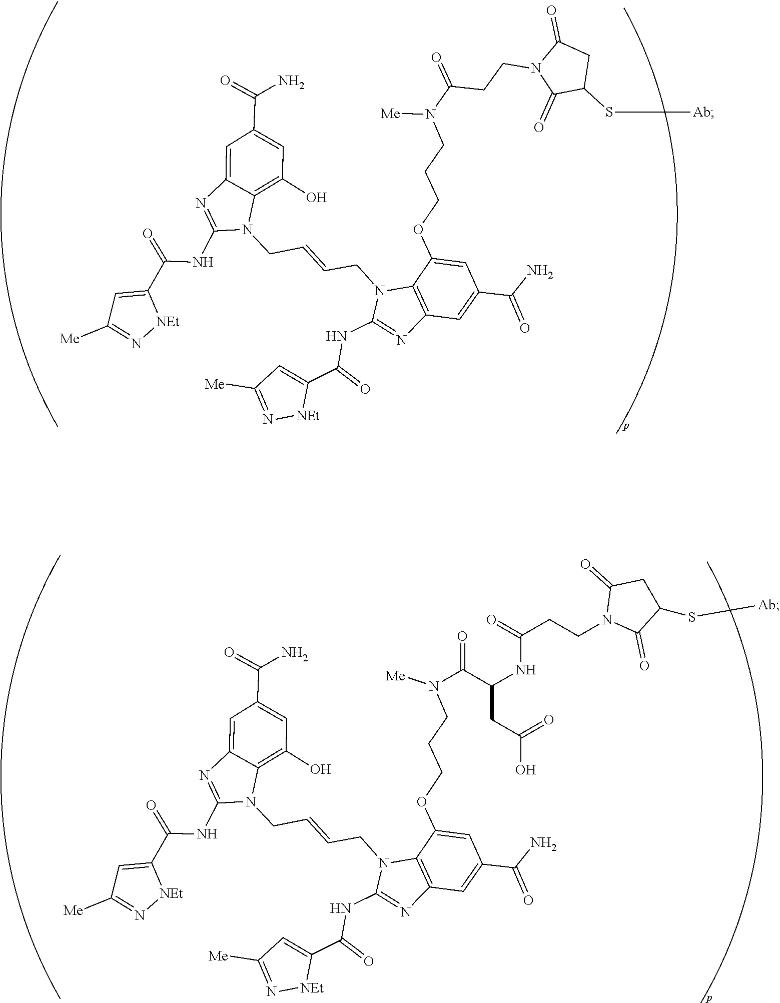

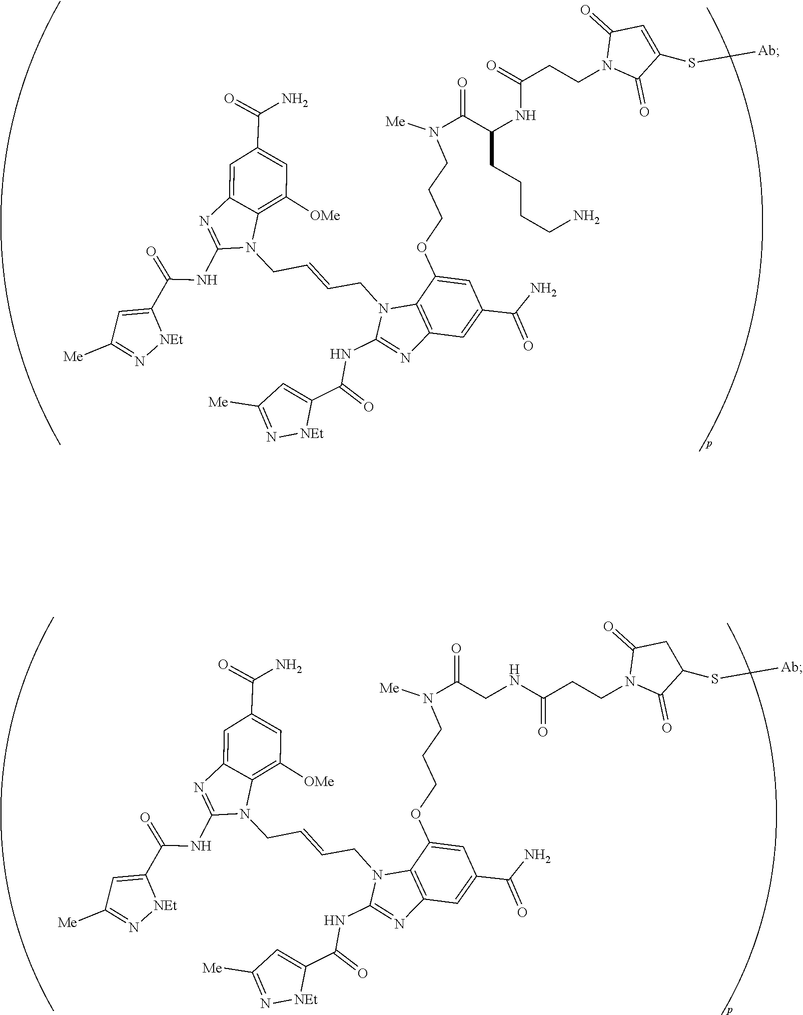

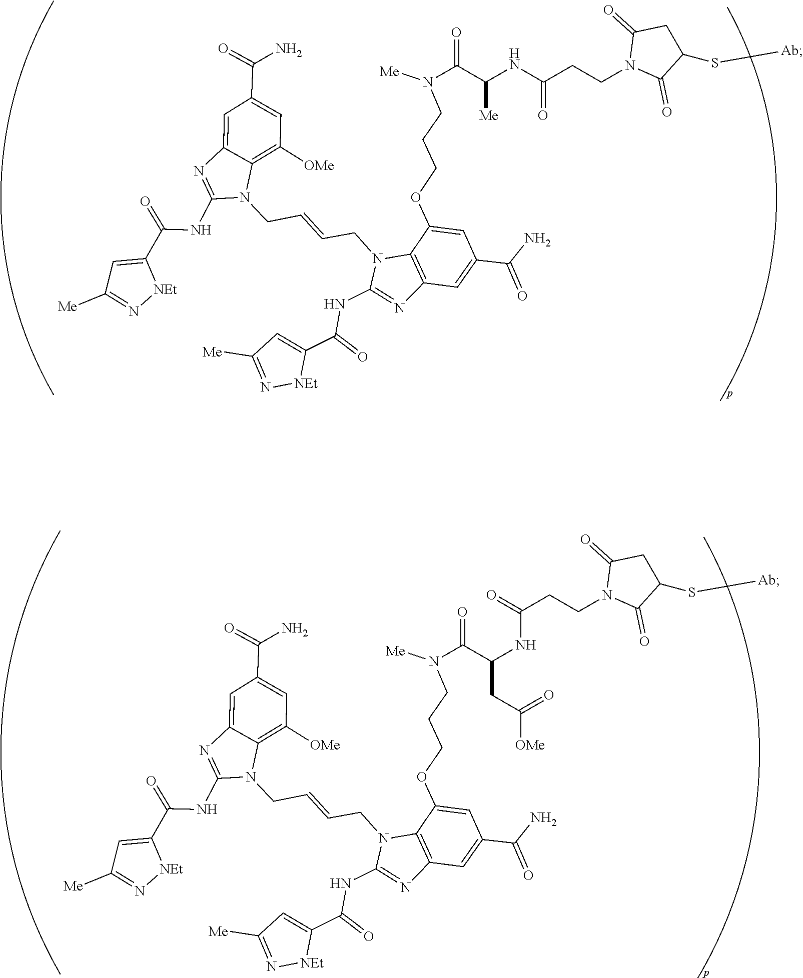

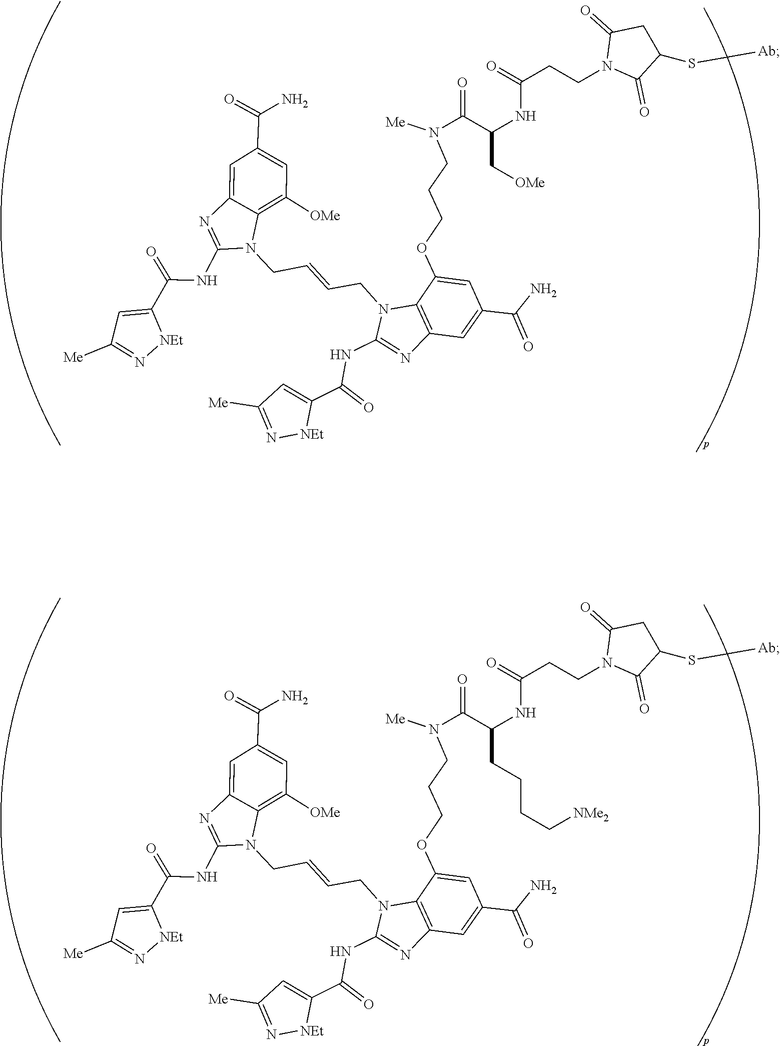

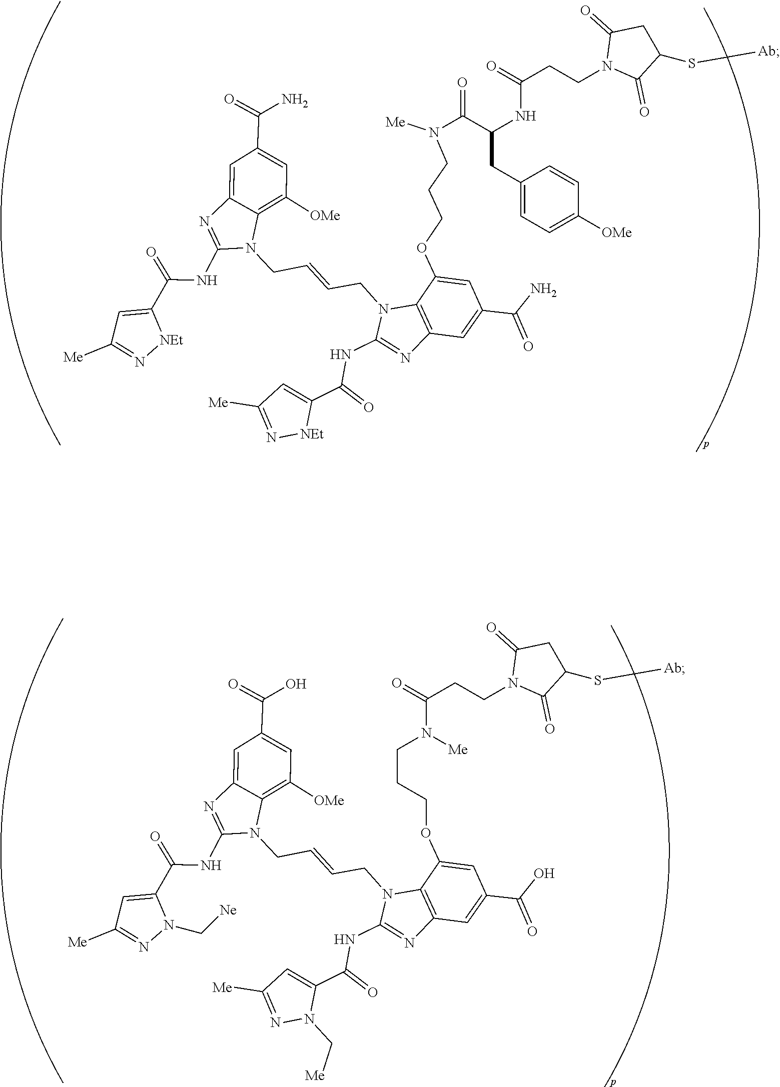

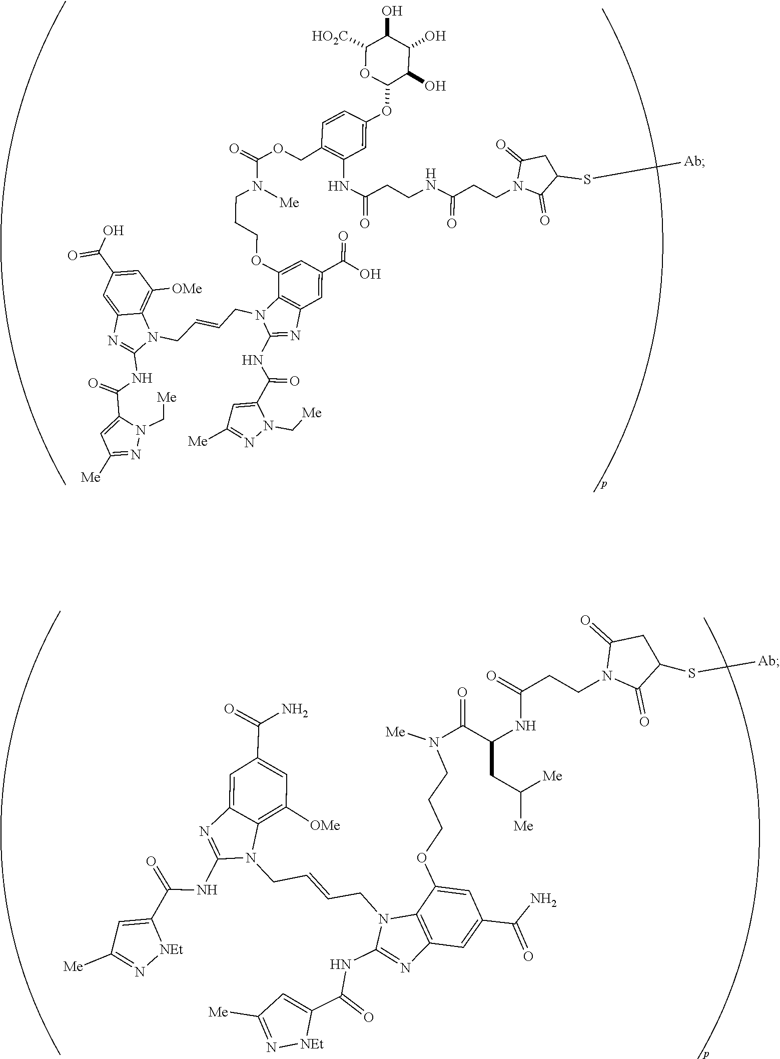

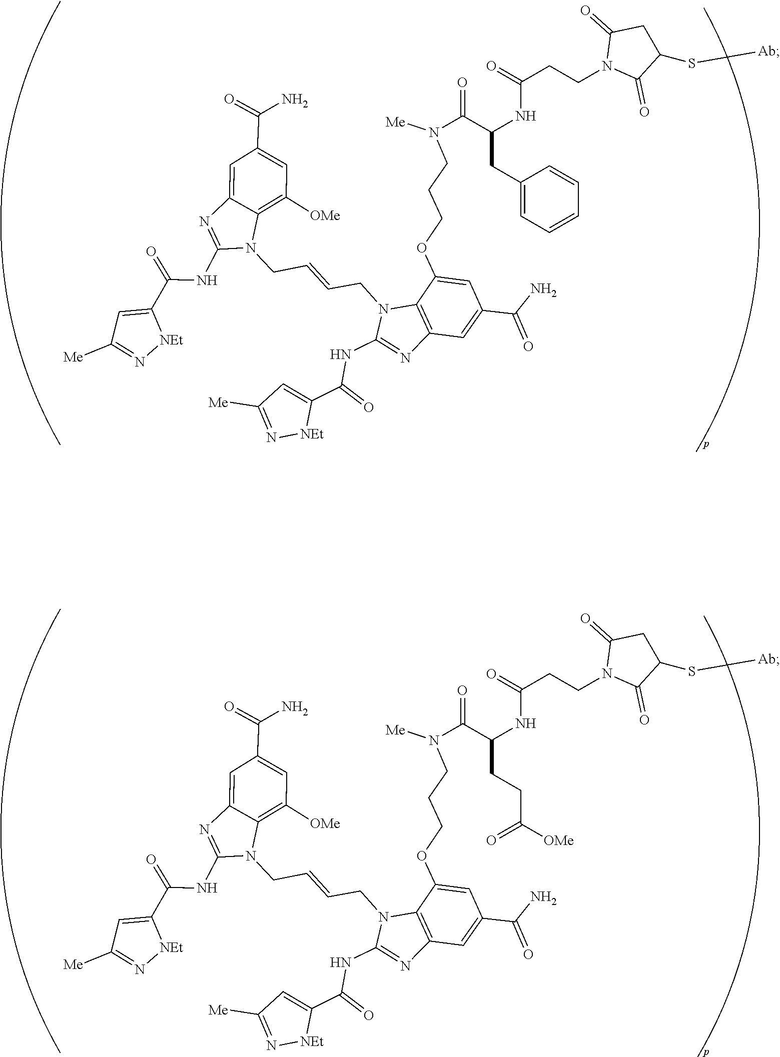

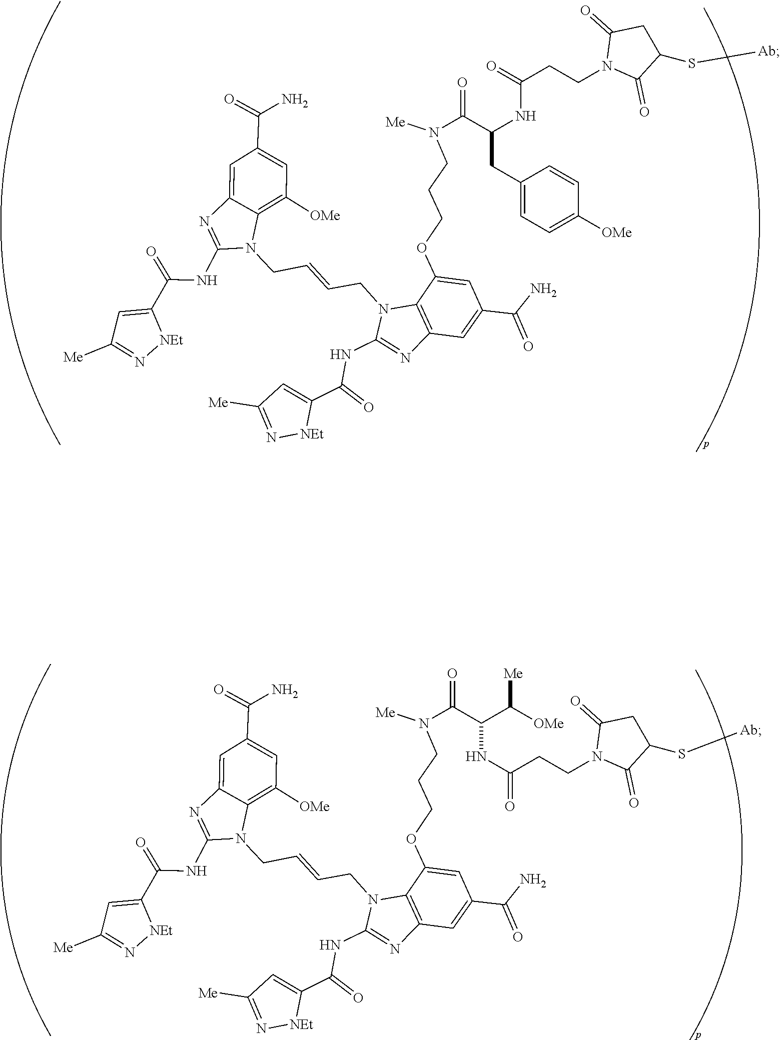

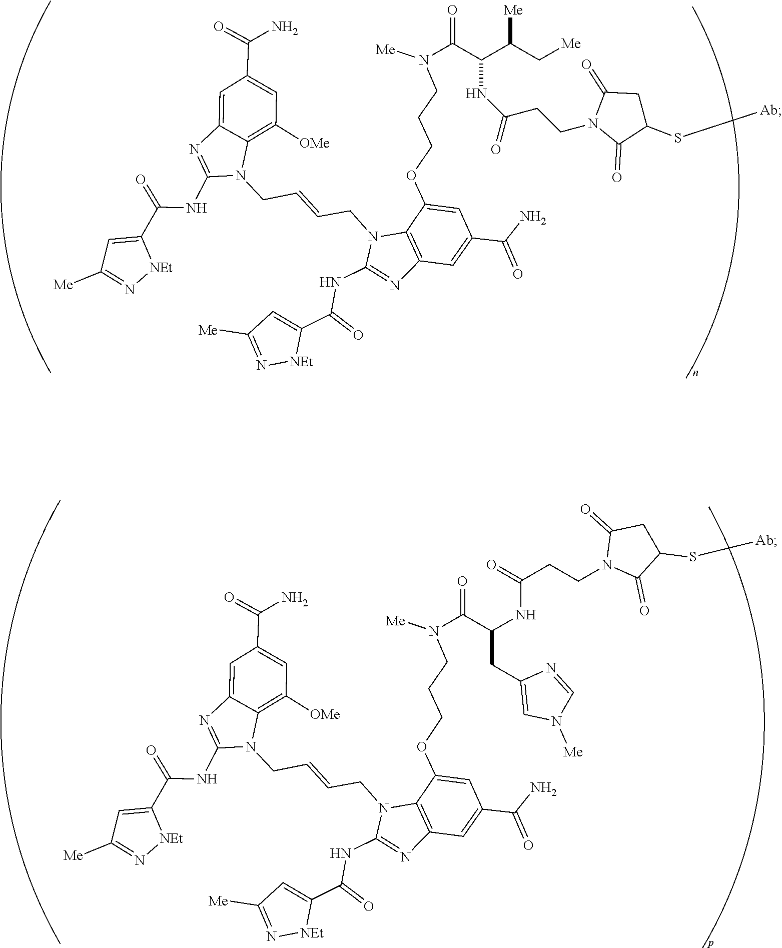

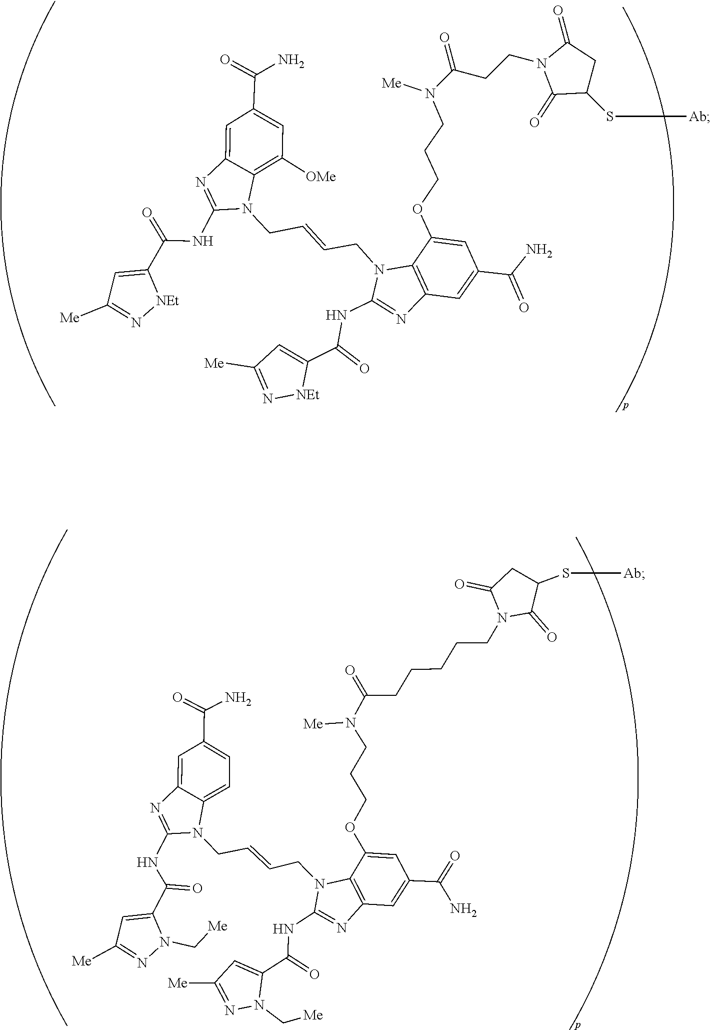

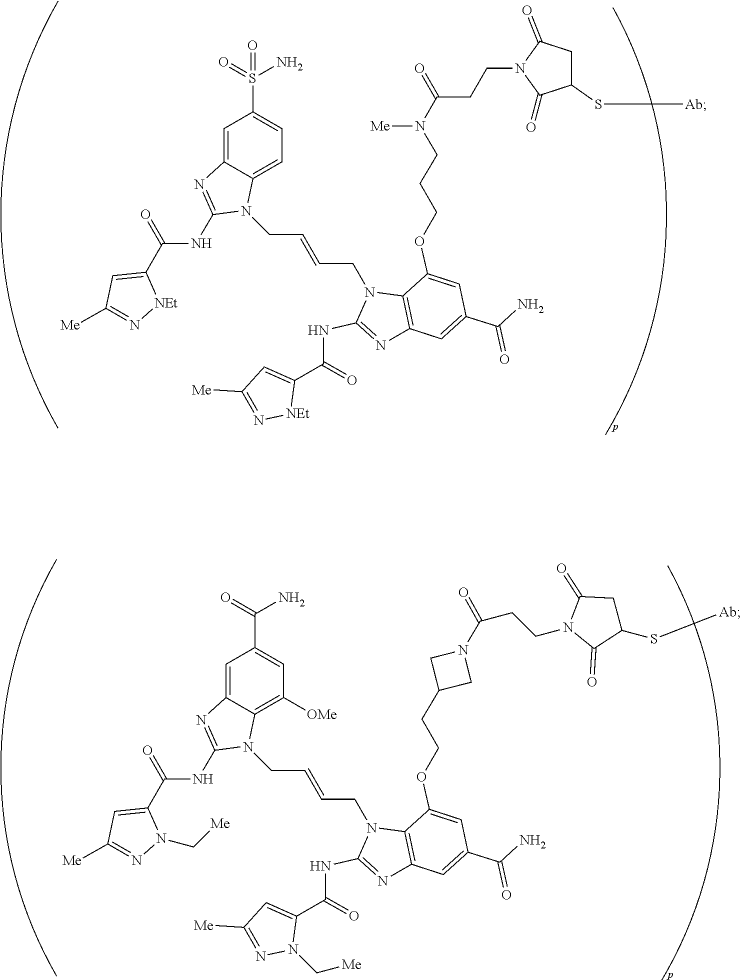

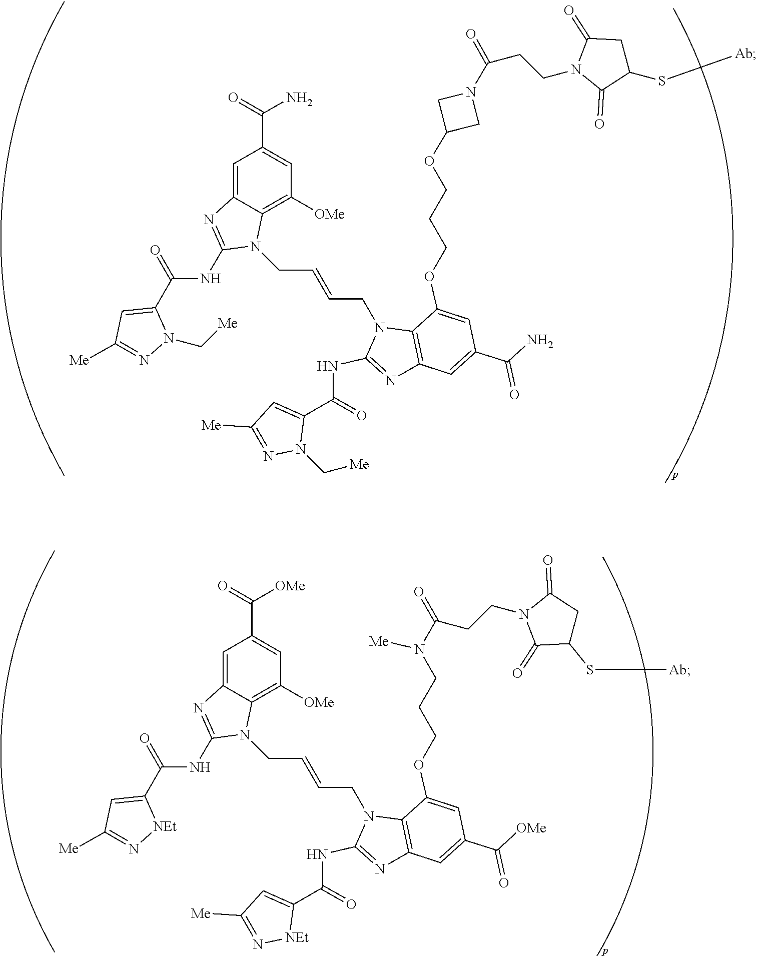

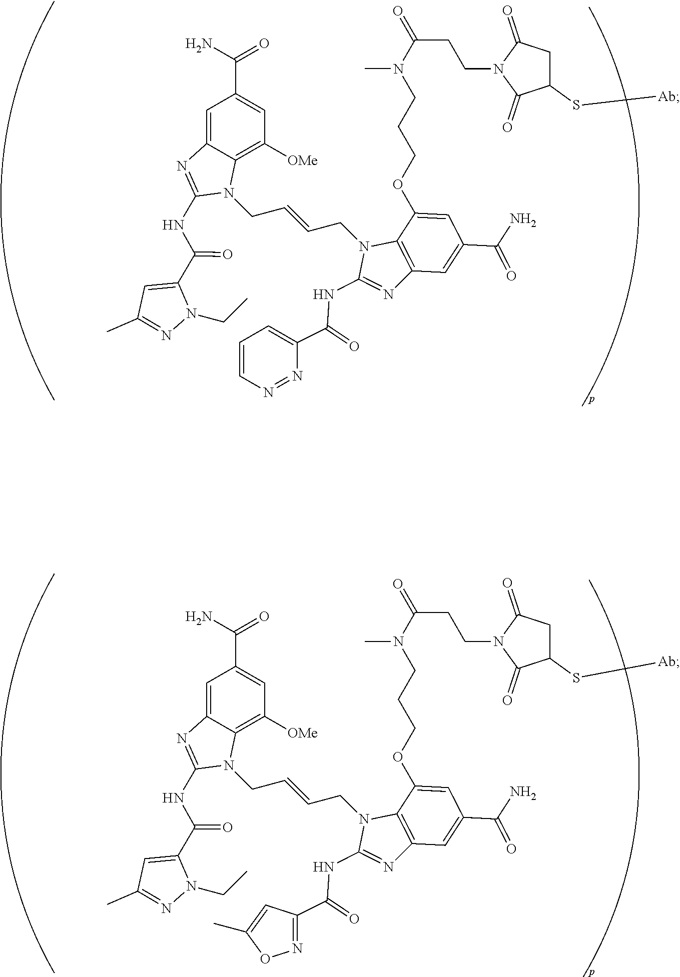

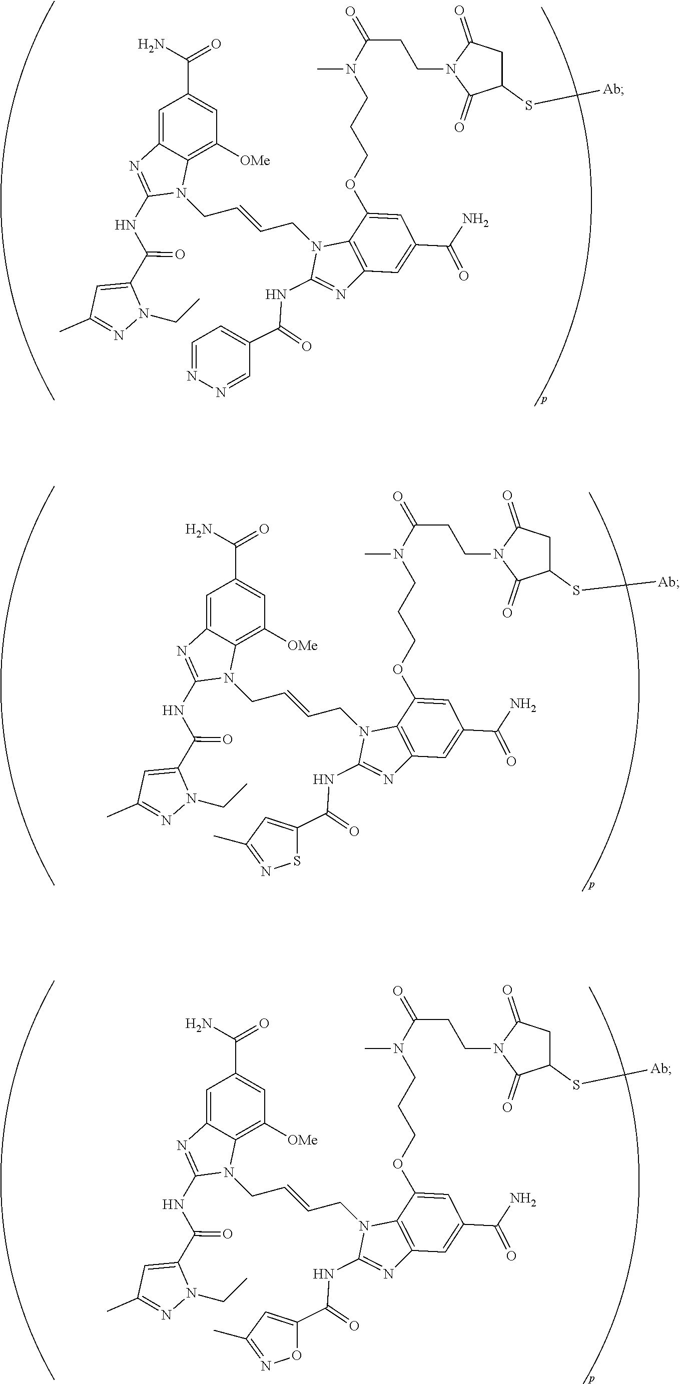

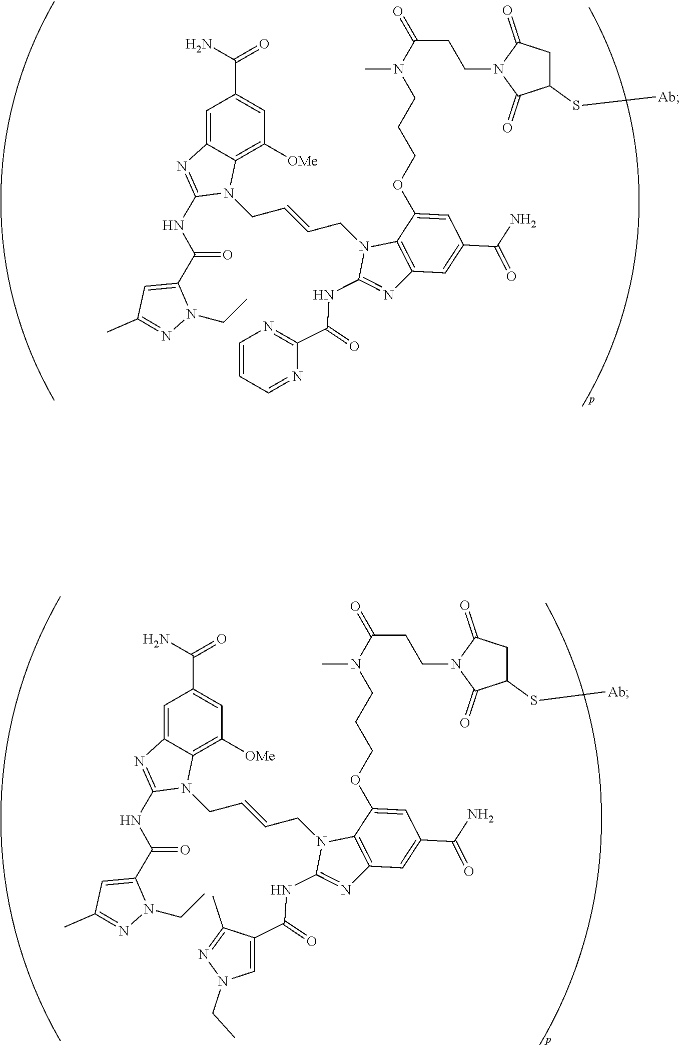

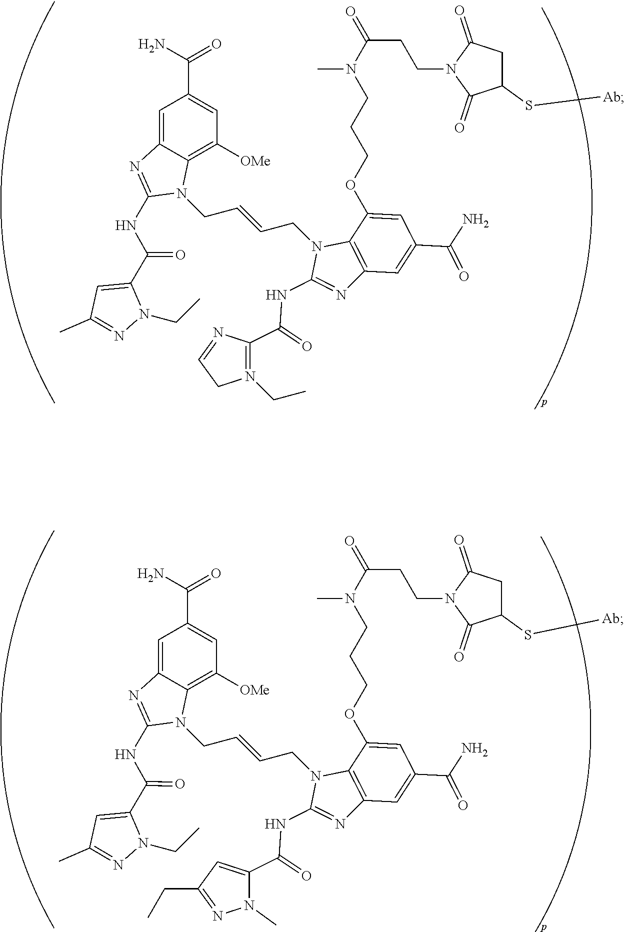

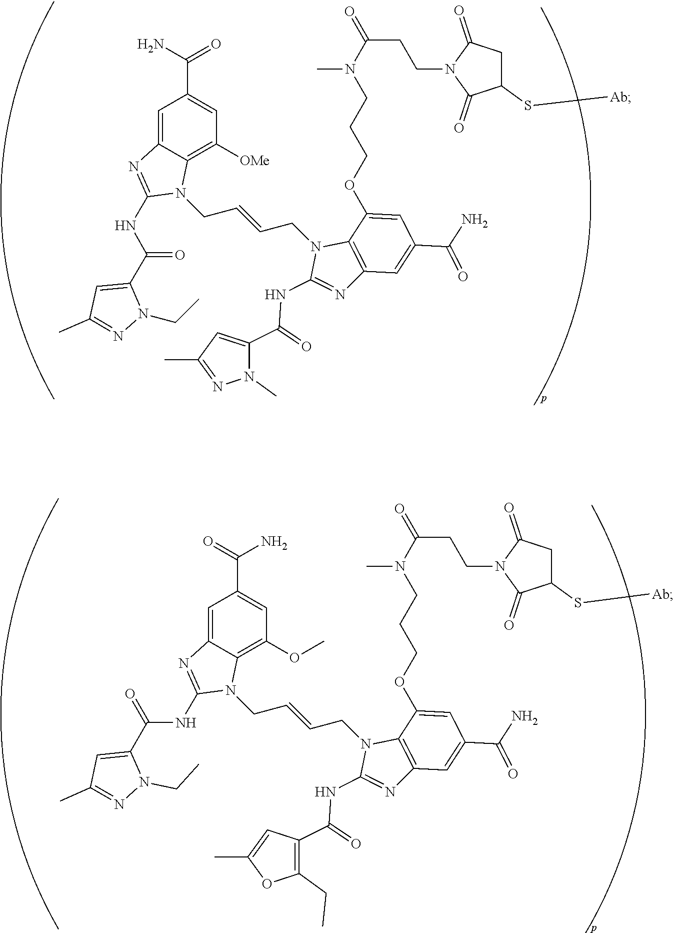

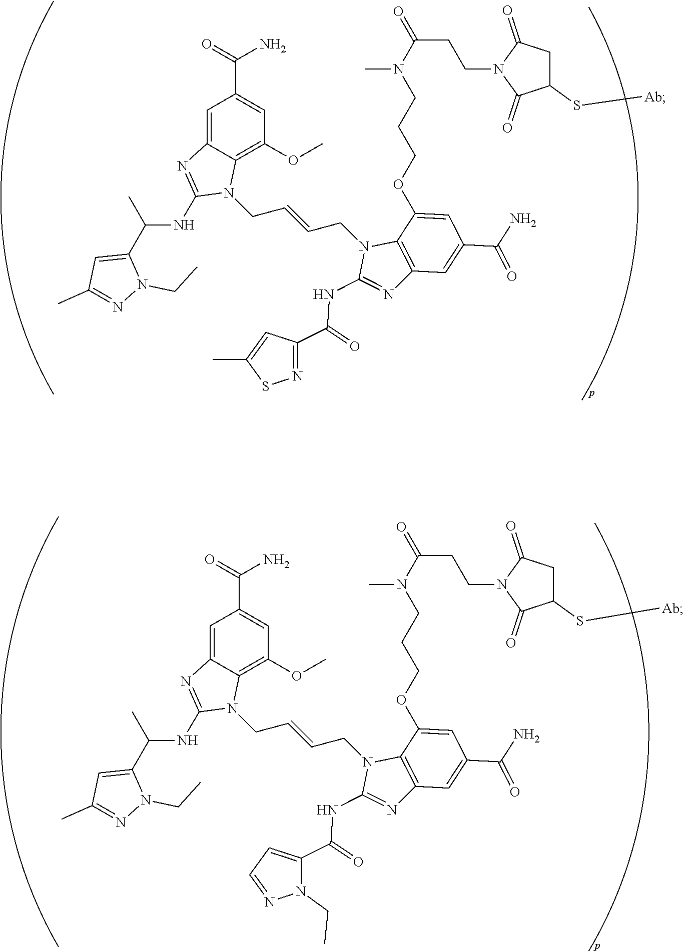

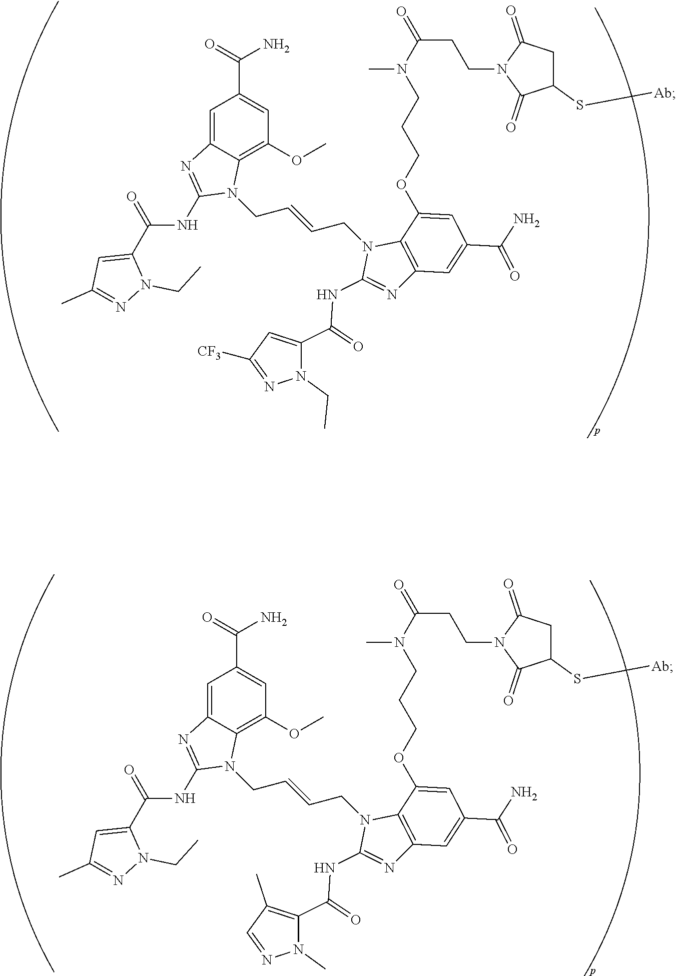

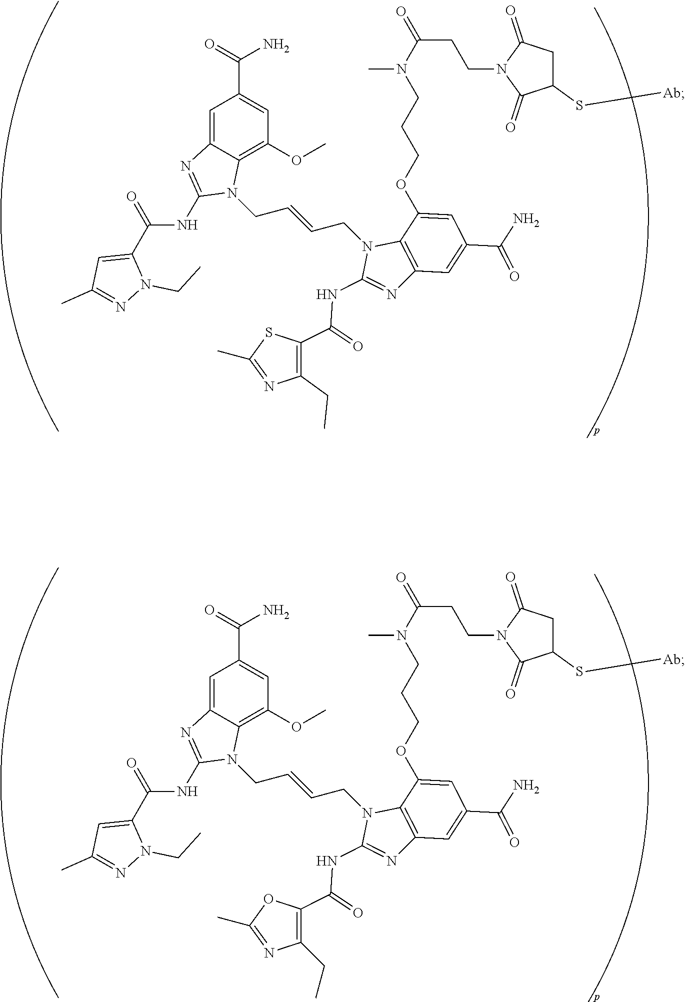

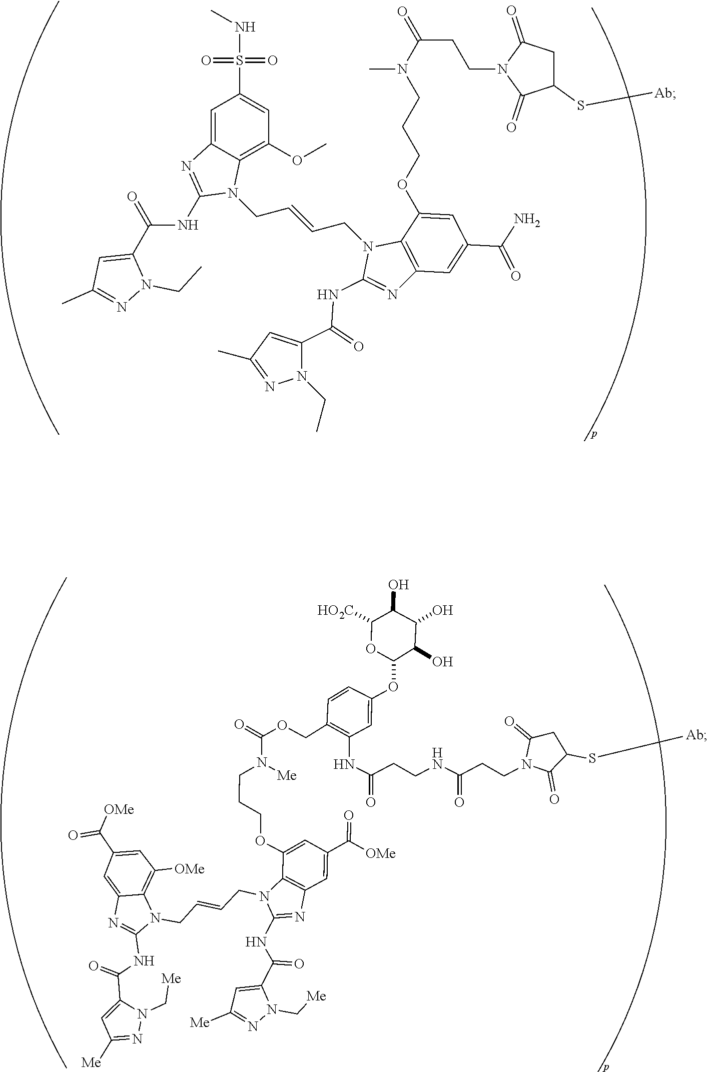

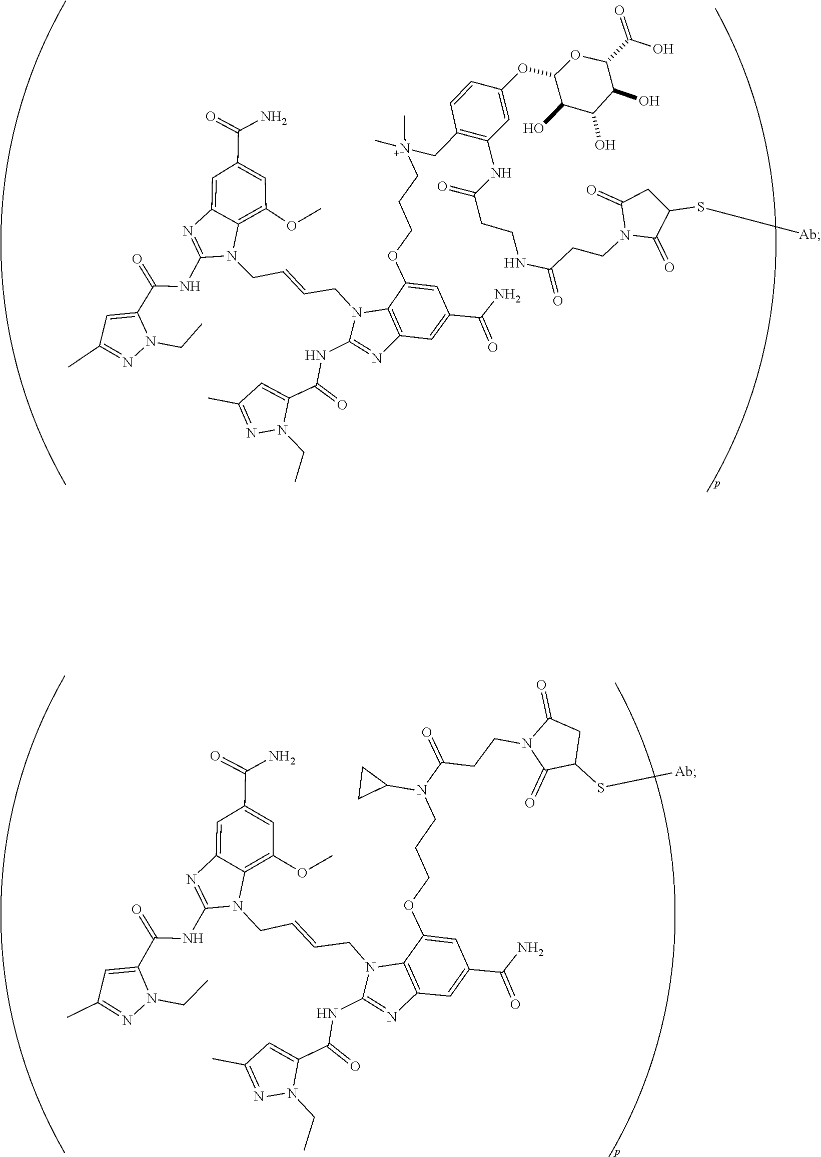

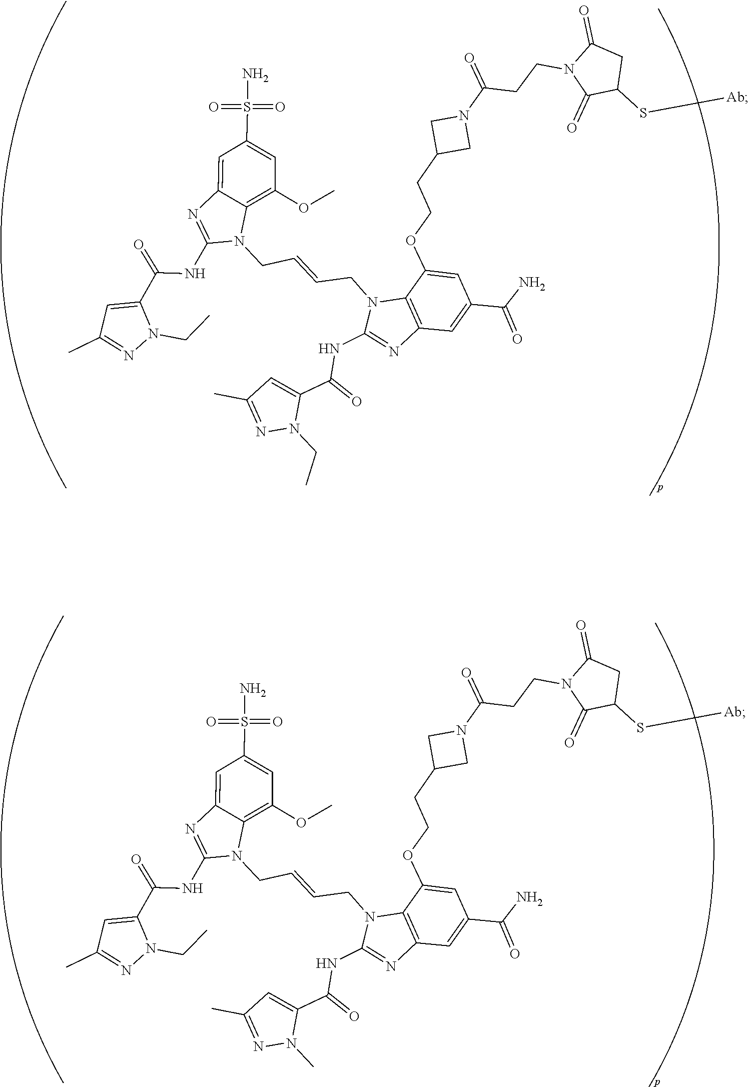

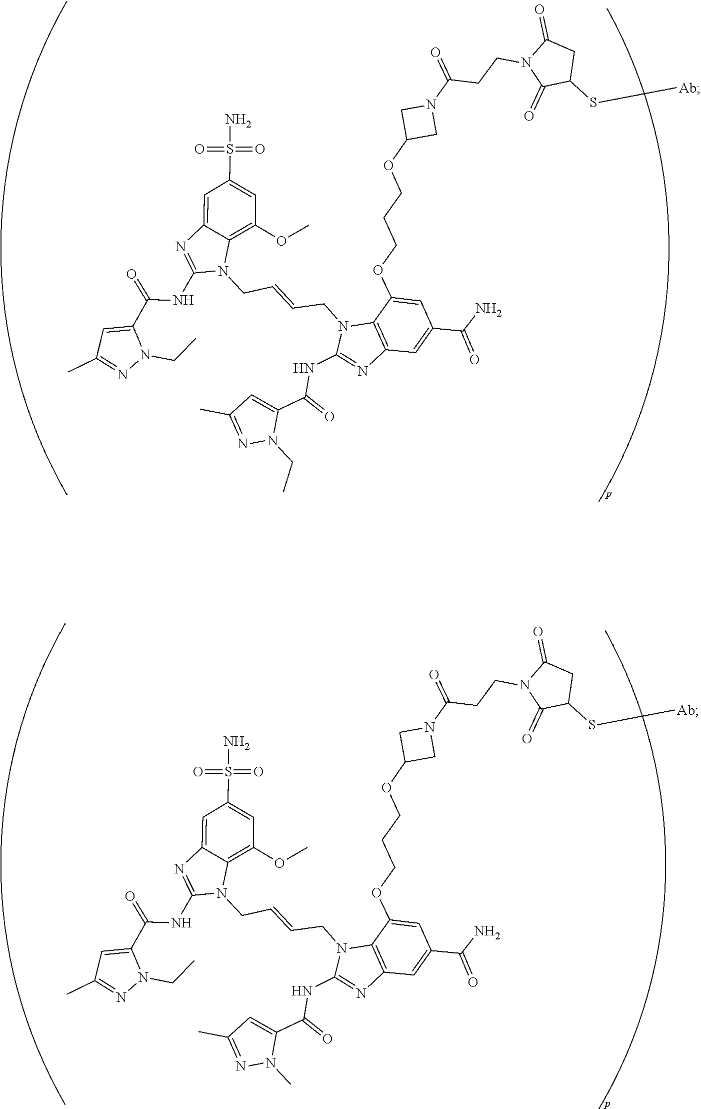

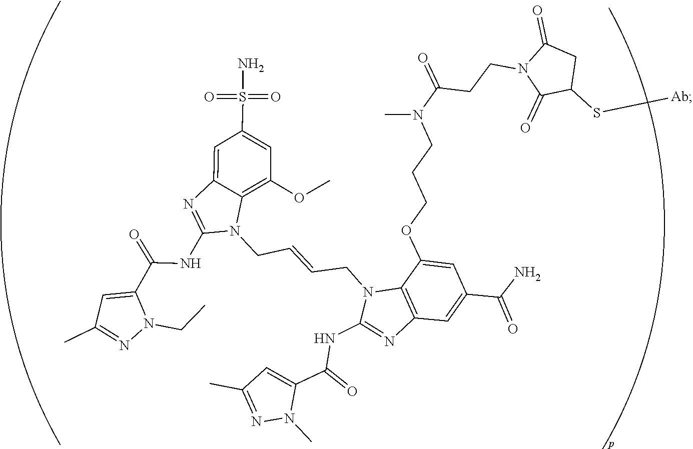

and pharmaceutically acceptable salts thereof,
wherein:
   Ab is an antibody; and
   each subscript p is independently an integer from 2 to 8.

### Antibodies

In some embodiments, an antibody is a polyclonal antibody. In some embodiments, an antibody is a monoclonal antibody. In some embodiments, an antibody is chimeric. In some embodiments, an antibody is humanized. In some embodiments, an antibody is fully human. In some embodiments, an antibody is an antigen binding fragment.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies and is not to be construed as requiring production of the antibody by any particular method.

Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies may be made by any of numerous techniques known in the art (e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16).

In some embodiments, an antibody includes a functionally active fragment, derivative or analog of an antibody that binds specifically to target cells (e.g., cancer cell antigens) or other antibodies bound to cancer cells or matrix. In this regard, “functionally active” means that the fragment, derivative or analog is able to bind specifically to target cells. To determine which CDR sequences bind the antigen, synthetic peptides containing the CDR sequences are typically used in binding assays with the antigen by any binding assay method known in the art (e.g., the Biacore assay) (See, e.g., Kabat et al., 1991, Sequences of Proteins of Immunological Interest, Fifth Edition, National Institute of Health, Bethesda, Md; Kabat E et al., 1980, J. Immunology 125(3):961-969).

Additionally, recombinant antibodies, such as chimeric and humanized monoclonal antibodies, comprising both human and non-human portions, which are typically obtained using standard recombinant DNA techniques, are useful antibodies. A chimeric antibody is a molecule in which different portions are derived from different animal species, such as for example, those having a variable region derived from a murine monoclonal and a constant region derived from a human immunoglobulin. See, e.g., U.S. Pat. Nos. 4,816,567; and 4,816,397, which are incorporated herein by reference in their entireties. Humanized antibodies are antibody molecules from non-human species having one or more CDRs from the non-human species and a framework region from a human immunoglobulin molecule. See, e.g., U.S. Pat. No. 5,585,089, which is incorporated herein by reference in its entirety. Such chimeric and humanized monoclonal antibodies can be produced by recombinant DNA techniques known in the art, for example using methods described in International Publication No. WO 87/02671; European Patent Publication No. 0 184 187; European Patent Publication No. 0 171 496; European Patent Publication No. 0 173 494; International Publication No. WO 86/01533; U.S. Pat. No. 4,816,567; European Patent Publication No. 012 023; Berter et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Cancer. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321: 522-525; Verhoeyan et al., 1988, Science 239:1534; and Beidler et al., 1988, J. Immunol. 141:4053-4060; each of which is incorporated herein by reference in its entirety.

In some embodiments, an antibody is a completely human antibody. In some embodiments, an antibody is produced using transgenic mice that are incapable of expressing endogenous immunoglobulin heavy and light chain genes, but which are capable of expressing human heavy and light chain genes.

In some embodiments, an antibody is an intact or fully-reduced antibody. The term ‘fully-reduced’ is meant to refer to an antibody in which all four inter-chain disulfide linkages have been reduced to provide eight thiols that can be attached to a linker (L).

Attachment to an antibody can be via thioether linkages from native and/or engineered cysteine residues, or from an amino acid residue engineered to participate in a cycloaddition reaction (such as a click reaction) with the corresponding linker intermediate. See, e.g., Maerle, et al., PLOS One 2019: 14(1); e0209860. In some embodiments, an antibody is an intact or fully-reduced antibody, or is an antibody bearing engineered an cysteine group that is modified with a functional group that can participate in, for example, click chemistry or other cycloaddition reactions for attachment of other components of the ADC as described herein (e.g., Diels-Alder reactions or other [3+2] or [4+2] cycloadditions).

Antibodies that bind specifically to a cancer cell antigen are available commercially or produced by any method known to one of skill in the art such as, e.g., chemical synthesis or recombinant expression techniques. The nucleotide sequences encoding antibodies that bind specifically to a cancer cell antigen are obtainable, e.g., from the GenBank database or similar database, literature publications, or by routine cloning and sequencing.

In some embodiments, the antibody can be used for the treatment of a cancer (e.g., an antibody approved by the FDA and/or EMA). Antibodies that bind specifically to a cancer cell antigen are available commercially or produced by any method known to one of skill in the art such as, e.g., recombinant expression techniques. The nucleotide sequences encoding antibodies that bind specifically to a cancer cell antigen are obtainable, e.g., from the GenBank database or similar database, literature publications, or by routine cloning and sequencing.

In some embodiments, an antibody can bind specifically to a receptor or a receptor complex expressed on lymphocytes. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein.

In some embodiments, an antibody can bind specifically to a cancer cell antigen. It will be understood that the antibody component in an ADC is an antibody in residue form such that “Ab” in the ADC structures described herein incorporates the structure of the antibody.

Non-limiting examples of antibodies that can be used for treatment of cancer and antibodies that bind specifically to tumor associated antigens are disclosed in Franke, A. E., Sievers, E. L., and Scheinberg, D. A., “Cell surface receptor-targeted therapy of acute myeloid leukemia: a review” Cancer Biother Radiopharm. 2000, 15, 459-76; Murray, J. L., “Monoclonal antibody treatment of solid tumors: a coming of age” Semin Oncol. 2000, 27, 64-70; Breitling, F., and Dubel, S., Recombinant Antibodies, John Wiley, and Sons, New York, 1998, each of which is hereby incorporated by reference in its entirety.

Embodiments of antibodies that bind to one or more of cancer cell antigens and immune cell antigens are provided below.

Non-limiting examples of target antigens and associated antibodies useful for the treatment of cancer and antibodies that bind specifically to cancer cell antigens (also called tumor antigens), include B7-DC (e.g., Catalog #PA5-20344); BCMA; B7-H3 (e.g., enoblituzumab, omburtamab, MGD009, MGC018, DS-7300); B7-H4 (e.g., Catalog #14-5949-82); B7-H6 (e.g., Catalog #12-6526-42); B7-H7; C5 complement (e.g., BCD-148; CAN106); CA-125; CA9 (e.g., girentuximab); CCR8 (e.g., JTX-1811); CLEC12A (e.g., tepoditamab); CSPG4 (e.g., U.S. Pat. No. 10,822,427); CCNB1; DDR1; de2-7 EGFR (e.g., MAb 806); DPEP1; DR4 (e.g., mapatumumab); endosialin (e.g., ontuxizumab); ENPP1; EPCAM (e.g., adecatumumab); EPHA2; ERBB2 (e.g., trastuzumab); ERBB3; ERVMER34-1; FAP (e.g., sibrotuzumab); FasL; FGFR2 (e.g., aprutumab); FGFR4 (e.g., MM-161); FLT3 (e.g., 4G8SDIEM); FBP; FucGM1 (e.g., BMS-986012); FZD8; G250; GAGE; GD2 (e.g., dinutuximab); gpNMB (e.g., glembatumumab); GPR87; GUCY2C (e.g., indusatumab); HAVCR2; IDO1; ITGB6; ITGB8; L1CAM (e.g., JCAR023); MRC1 (e.g., ThermoFisher Catalog #12-2061-82); ML-IAP (e.g., 88C570, ThermoFisher Catalog #40958); NT5E (e.g., 7G2, ThermoFisher Catalog #41-0200); OY-TES1; p53; p53mutant; PAX5; PDPN (e.g., ThermoFisher Catalog #14-5381-82); VSIR (e.g., ThermoFisher Catalog #PA5-52493); Dectin2 (e.g., ThermoFisher Catalog #MA5-16250); PAX3 (e.g., GT1210, ThermoFisher Catalog #MA5-31583); Sialyl-Thomsen-nouveau-antigen (e.g., Eavarone et al. PLoS One, 2018; 13(7): e0201314); PDGFR-B (e.g., rinucumab); ADAM12 (e.g., Catalog #14139-1-AP); ADAM9 (e.g., IMGC936); AFP (e.g., ThermoFisher Catalog #PA5-25959); AGR2 (e.g., ThermoFisher Catalog #PA5-34517); AKAP-4 (e.g., Catalog #PA5-52230); androgen receptor (e.g., ThermoFisher Catalog #MA5-13426); ALPP (e.g., Catalog #MA5-15652); CD44 (e.g., RG7356); AMHR2 (e.g., ThermoFisher Catalog #PA5-13902); ANTXR1 (e.g., Catalog #MA1-91702); ARTN (e.g., ThermoFisher Catalog #PA5-47063); αVβ6; CA19-9 (e.g., AbGn-7; MVT-5873); carcinoembryonic antigen (e.g., arcitumomab; cergutuzumab; amunaleukin; labetuzumab); CD115 (e.g., axatilimab; cabiralizumab; emactuzumab); CD137 (e.g., ADG106; CTX-471); CD147 (e.g., gavilimomab; metuzumab); CD155 (e.g., U.S. Publication No. 2018/0251548); CD274 (e.g., adebrelimab; atezolizumab; garivulimab); CDCP1 (e.g., RG7287); CDH3 (e.g., PCA062); CDH6 (e.g., HKT288); CEACAM1; CEACAM6; CLDN18.1 (e.g., zolbetuximab); CLDN18.2 (e.g., zolbetuximab); CLPTM1L; CS-1 (e.g., tigatuzumab); GD3 (e.g., mitumomab); HLA-G (e.g., TTX-080); IL1RAP (e.g., nidanilimab); LAG-3 (e.g., encelimab); LY6G6D (e.g., PA5-23303); LYPD1 (e.g., ThermoFisher Catalog #PA5-26749); MAD-CT-2; MAGEA3 (e.g., ThermoFisher Catalog #60054-1-IG); MAGEA4 (e.g., Catalog #MA5-26117); MAGEC2 (e.g., ThermoFisher Catalog #PA5-64010); MLANA (e.g., Catalog #MA5-15237); MELTF (e.g., ThermoFisher Catalog #H00004241-M04A); MSLN (e.g., 5B2, Catalog #MA5-11918); MUC1 (e.g., MH1 (CT2), ThermoFisher Catalog #MA5-11202); MUC5AC (e.g., 45M1, Catalog #MA5-12178); MYCN (e.g., NCM-II 100, ThermoFisher Catalog #MA1-170); NCAM1 (e.g., ThermoFisher Catalog #MA5-11563); Nectin-4 (e.g., enfortumab); NY-BR-1 (e.g., NY-BR-1 No. 2, Catalog #MA5-12645); PSMA (e.g., BAY 2315497); PSA (e.g., ThermoFisher Catalog #PA1-38514; Daniels-Wells et al. BMC Cancer, 2013; 13:195); PSCA (e.g., AGS-1C4D4); PTK7 (e.g., cofetuzumab); PVRIG; Ras mutant (e.g., Shin et al. Sci Adv. 2020; 6(3):eaay2174); RET (e.g., WO2020210551); RGS5 (e.g., TF-TA503075); RhoC (e.g., ThermoFisher Catalog PA5-77866); ROR2 (e.g., BA3021); ROS1 (e.g., WO2019107671); SART3 (e.g., TF 18025-1-AP); SLC12A2 (e.g., ThermoFisher Catalog #13884-1-AP); SLC38A1 (e.g., ThermoFisher Catalog #12039-1-AP); SLC39A6 (e.g., ladiratuzumab); SLC44A4 (e.g., ASG-5ME); SLC7A11 (e.g., ThermoFisher Catalog #PA1-16893); SLITRK6 (e.g., sirtratumab); SSX2 (e.g., ThermoFisher Catalog #MA5-24971); survivin (e.g., PA1-16836); TACSTD2 (e.g., PA5-47074); TAG-72 (e.g., MA1-25956); TIGIT (e.g., etigilimab); TM4SF5 (e.g., 18239-1-AP); TMPRSS11D (e.g., PA5-30927); TNFRSF12 (e.g., BAY-356); TRAIL (e.g., Catalog #12-9927-42); Trem2 (e.g., PY314); TRP-2 (e.g., PA5-52736); uPAR (e.g., ATN-658); UPK1B (e.g., ThermoFisher Catalog #PA5-56863); UPK2 (e.g., ThermoFisher Catalog #PA5-60318); UPK3B (e.g., ThermoFisher Catalog #PA5-52696); VEGF (e.g., GNR-011); VEGFR2 (e.g., gentuximab); CD44 (e.g., RG7356); WT1 (e.g., ThermoFisher Catalog #MA5-32215); XAGE1 (e.g., ThermoFisher Catalog #PA5-46413); CTLA4 (e.g., ipilimumab); Sperm protein 17 (e.g., BS-5754R); TLR2/4/1 (e.g., tomaralimab); B7-1 (e.g., galiximab); ANXA1 (e.g., Catalog #71-3400); BCR-ABL; CAMPATH-1 (e.g., alemtuzumab; ALLO-647; ANT1034); CD123 (e.g., BAY-943; CSL360); CD19 (e.g., ALLO-501); CD20 (e.g., divozilimab; ibritumomab); CD30 (e.g., iratumumab); CD33 (e.g., lintuzumab; BI 836858; AMG 673); CD352 (e.g., SGN-CD352A); CD37 (e.g., lilotomab; GEN3009); CD40 (e.g., dacetuzumab; lucatumumab); CD45 (e.g., apamistamab); CD48 (e.g., SGN-CD48A); CXCR4 (e.g., ulocuplumab); ETV6-AML (e.g., Catalog #PA5-81865); ROR1 (e.g., cirmtuzumab); CD74 (e.g., milatuzumab); SIT1 (e.g., PA5-53825); SLAMF7 (e.g., Elotuzumab); Axl (e.g., BA3011; tilvestamab); Siglecs 1-16 (see, e.g., Angata et al. Trends Pharmacol Sci. 2015; 36(10): 645-660); SIRPa (e.g., Catalog #17-1729-42); SIRPg (e.g., PA5-104381); OX40 (e.g., ABM193); PROM1 (e.g., Catalog #14-1331-82); TMEM132A (e.g., Catalog #PA5-62524); TMEM40 (e.g., PA5-60636); PD-1 (e.g., balstilimab; budigalimab; geptanolimab); ALK (e.g., DLX521); CCR4 (e.g., AT008; mogamulizumab-kpkc); CD27 (e.g., varlilumab); CD278 (e.g., feladilimab; vopratelimab); CD32 (e.g., mAb 2B6); CD47 (e.g., letaplimab; magrolimab); and CD70 (e.g., cusatuzumab).

In some embodiments, an antibody can bind specifically to a cancer cell antigen associated with a solid tumor and/or a hematological cancer. Non-limiting examples of target antigens and associated antibodies that bind specifically to cancer cell antigens associated with a solid tumor and/or a hematological cancer target antigen include Axl (e.g., BA3011; tilvestamab); B7-H3 (e.g., enoblituzumab, omburtamab, MGD009, MGC018, DS-7300); B7-H4 (e.g., Catalog #14-5949-82); B7-H6 (e.g., Catalog #12-6526-42); B7-H7; Siglecs 1-16 (see, e.g., Angata et al. Trends Pharmacol Sci. 2015; 36(10): 645-660); SIRPa (e.g., Catalog #17-1729-42); SIRPg (e.g., PA5-104381); OX40 (e.g., ABM193); PROM1 (e.g., Catalog #14-1331-82); TMEM132A (e.g., Catalog #PA5-62524); TMEM40 (e.g., PA5-60636); PD-1 (e.g., balstilimab; budigalimab; geptanolimab); ALK (e.g., DLX521); CCR4 (e.g., AT008; mogamulizumab-kpkc); CD27 (e.g., varlilumab); CD278 (e.g., feladilimab; vopratelimab); CD32 (e.g., mAb 2B6); CD47 (e.g., letaplimab; magrolimab); and CD70 (e.g., cusatuzumab).

In some embodiments, an antibody can bind specifically to a cancer cell antigen associated with a solid tumor. Non-limiting examples of target antigens and associated antibodies that bind specifically to solid-tumor-associated target antigens include PAX3 (e.g., GT1210, ThermoFisher Catalog #MA5-31583); Sialyl-Thomsen-nouveau-antigen (e.g., Eavarone et al. PLoS One. 2018; 13(7): e0201314); PDGFR-B (e.g., rinucumab); ADAM12 (e.g., Catalog #14139-1-AP); ADAM9 (e.g., IMGC936); AFP (e.g., ThermoFisher Catalog #PA5-25959); AGR2 (e.g., ThermoFisher Catalog #PA5-34517); AKAP-4 (e.g., Catalog #PA5-52230); androgen receptor (e.g., ThermoFisher Catalog #MA5-13426); ALPP (e.g., Catalog #MA5-15652); CD44 (e.g., RG7356); AMHR2 (e.g., ThermoFisher Catalog #PA5-13902); ANTXR1 (e.g., Catalog #MA1-91702); ARTN (e.g., ThermoFisher Catalog #PA5-47063); αVβ6; CA19-9 (e.g., AbGn-7; MVT-5873); carcinoembryonic antigen (e.g., arcitumomab; cergutuzumab; amunaleukin; labetuzumab); CD115 (e.g., axatilimab; cabiralizumab; emactuzumab); CD137 (e.g., ADG106; CTX-471); CD147 (e.g., gavilimomab; Metuzumab); CD155 (e.g., U.S. Publication No. 2018/0251548); CD274 (e.g., adebrelimab; atezolizumab; garivulimab); CDCP1 (e.g., RG7287); CDH3 (e.g., PCA062); CDH6 (e.g., HKT288); CEACAM1; CEACAM6); CLDN18.1 (e.g., zolbetuximab); CLDN18.2 (e.g., zolbetuximab); CLPTM1L; CS-1 (e.g., tigatuzumab); GD3 (e.g., mitumomab); HLA-G (e.g., TTX-080); IL1RAP (e.g., nidanilimab); LAG-3 (e.g., encelimab); LY6G6D (e.g., PA5-23303); LYPD1 (e.g., ThermoFisher Catalog #PA5-26749); MAD-CT-2; MAGEA3 (e.g., ThermoFisher Catalog #60054-1-IG); MAGEA4 (e.g., Catalog #MA5-26117); MAGEC2 (e.g., ThermoFisher Catalog #PA5-64010); MLANA (e.g., Catalog #MA5-15237); MELTF (e.g., ThermoFisher Catalog #H00004241-M04A); MSLN (e.g., 5B2, Catalog #MA5-11918); MUC1 (e.g., MH1 (CT2), ThermoFisher Catalog #MA5-11202); MUC5AC (e.g., 45M1, Catalog #MA5-12178); MYCN (e.g., NCM-II 100, ThermoFisher Catalog #MA1-170); NCAM1 (e.g., ThermoFisher Catalog #MA5-11563); Nectin-4 (e.g., enfortumab); NY-BR-1 (e.g., NY-BR-1 No. 2, Catalog #MA5-12645); PSMA (e.g., BAY 2315497); PSA (e.g., ThermoFisher Catalog #PA1-38514; Daniels-Wells et al. BMC Cancer 2013; 13:195); PSCA (e.g., AGS-1C4D4); PTK7 (e.g., cofetuzumab); PVRIG; Ras mutant (e.g., Shin et al. Sci Adv. 2020; 6(3):eaay2174); RET (e.g., WO2020210551); RGS5 (e.g., TF-TA503075); RhoC (e.g., ThermoFisher Catalog PA5-77866); ROR2 (e.g., BA3021); ROS1 (e.g., WO2019107671); SART3 (e.g., TF 18025-1-AP); SLC12A2 (e.g., ThermoFisher Catalog #13884-1-AP); SLC38A1 (e.g., ThermoFisher Catalog #12039-1-AP); SLC39A6 (e.g., ladiratuzumab); SLC44A4 (e.g., ASG-5ME); SLC7A11 (e.g., ThermoFisher Catalog #PA1-16893); SLITRK6 (e.g., sirtratumab); SSX2 (e.g., ThermoFisher Catalog #MA5-24971); survivin (e.g., PA1-16836); TACSTD2 (e.g., PA5-47074); TAG-72 (e.g., MA1-25956); TIGIT (e.g., etigilimab); TM4SF5 (e.g., 18239-1-AP); TMPRSS11D (e.g., PA5-30927); TNFRSF12 (e.g., BAY-356); TRAIL (e.g., Catalog #12-9927-42); Trem2 (e.g., PY314); TRP-2 (e.g., PA5-52736); uPAR (e.g., ATN-658); UPK1B (e.g., ThermoFisher Catalog #PA5-56863); UPK2 (e.g., ThermoFisher Catalog #PA5-60318); UPK3B (e.g., ThermoFisher Catalog #PA5-52696); VEGF (e.g., GNR-011); VEGFR2 (e.g., gentuximab); CD44 (e.g., RG7356); WT1 (e.g., ThermoFisher Catalog #MA5-32215); XAGE1 (e.g., ThermoFisher Catalog #PA5-46413); and CTLA4 (e.g., ipilimumab).

In some embodiments, an antibody can bind specifically to a cancer cell antigen associated with a hematological cancer. Non-limiting examples of target antigens and associated antibodies that bind specifically to hematological cancer cell target antigens include Sperm protein 17 (e.g., BS-5754R); TLR2/4/1 (e.g., Tomaralimab); B7-1 (e.g., galiximab); ANXA1 (e.g., Catalog #71-3400); BCR-ABL; CAMPATH-1 (e.g., alemtuzumab; ALLO-647; ANT1034); CD123 (e.g., BAY-943; CSL360); CD19 (e.g., ALLO-501); CD20 (e.g., divozilimab; ibritumomab); CD30 (e.g., iratumumab); CD33 (e.g., lintuzumab; BI 836858; AMG 673); CD352 (e.g., SGN-CD352A); CD37 (e.g., lilotomab; GEN3009); CD40 (e.g., dacetuzumab; lucatumumab); CD45 (e.g., apamistamab); CD48 (e.g., SGN-CD48A); CXCR4 (e.g., ulocuplumab); ETV6-AML (e.g., Catalog #PA5-81865); ROR1 (e.g., cirmtuzumab); CD74 (e.g., milatuzumab); SIT1 (e.g., PA5-53825); and SLAMF7 (e.g., elotuzumab).

In some embodiments, an antibody can be used that binds specifically to a target antigen (e.g., an antigen associated with a disease or disorder). Antibodies that bind specifically to a target antigen (e.g., an antigen associated with a disease or disorder) are available commercially or can be produced by any method known to one of skill in the art such as, e.g., recombinant expression techniques. The nucleotide sequences encoding antibodies that bind specifically to a target antigen (e.g., an antigen associated with a disease or disorder) are obtainable, e.g., from the GenBank database or similar database, literature publications, or by routine cloning and sequencing.

Non-limiting examples of target antigens and associated antibodies that bind specifically to target antigens (e.g., an antigen associated with a disease or disorder, or an antigen associated with an immune cell) include CD163 (e.g., TBI 304H); TIGIT (e.g., etigilimab); DCSIGN (see, e.g., International Publication No. WO2018134389); IFNAR1 (e.g., faralimomab); ASCT2 (e.g., idactamab); ULBP1/2/3/4/5/6 (e.g., PA5-82302); CLDN1 (e.g., INSERM anti-Claudin-1); CLDN2 (see, e.g., International Publication No. WO2018123949); IL-21R (e.g., PF-05230900); DCIR DCLK1 (see, e.g., International Publication No. WO2018222675); Dectini (see, e.g., U.S. Pat. No. 9,045,542); GITR (e.g., ragifilimab); ITGAV (e.g., abituzumab); LY9 (e.g., PA5-95601); MICA (e.g., 1E2C8, Catalog #66384-1-IG); MICB (e.g., Catalog #MA5-29422); NOX1 (e.g., Catalog #PA5-103220); CD2 (e.g., BTI-322; siplizumab); CD247 (e.g., AFM15); CD25 (e.g., basiliximab); CD28 (e.g., REGN5668); CD3 (e.g., otelixizumab; visilizumab); CD38 (e.g., felzartamab; AMG 424); CD3E (e.g., foralumab; teplizumab); CD5 (e.g., MAT 304; zolimomab aritox); ALPPL2 (e.g., Catalog #PA5-22336); B7-2 (e.g., Catalog #12-0862-82); B7-H3 (e.g., enoblituzumab, omburtamab, MGD009, MGC018, DS-7300); B7-H4 (e.g., Catalog #14-5949-82); B7-H6 (e.g., Catalog #12-6526-42); B7-H7; BAFF-R (e.g., Catalog #14-9117-82); BMPR2; BORIS; CD112 (see, e.g., U.S. Publication No. 20100008928); CD24 (see, e.g., U.S. Pat. No. 8,614,301); CD244 (e.g., R&D AF1039); CD30L (see, e.g., U.S. Pat. No. 9,926,373); CD3D; CD3G; CD79A (see, e.g., International Publication No. WO 2020252110); CD83 (e.g., CBT004); CD97; CDH17 (see, e.g., International Publication No. WO 2018115231); CLDN16; CLDN19; CYP1B1; DPEP3; DPP4; DSG2 (see, e.g., U.S. Pat. No. 10,836,823); EPHA receptors; epidermal growth factor; FAS; FGFR1 (e.g., RG7992); FGFR3 (e.g., vofatamab); FN1; FOLR1 (e.g., farletuzumab); FSHR; FZD5; GM2 (e.g., BIW-8962); GM3 (e.g., racotumomab); GPA33 (e.g., KRN330); GPC3 (e.g., codrituzumab); HAS3; HLA-E; HLA-F; HLA-DR; ICAM1; IFNAR2; IL13Ra2; IL-5R (e.g., benralizumab); KISS1R; LAMP1; LAYN; LCK; legumain; LILRB2; LILRB4; LMP2; MAD-CT-1; MAGEA1 (e.g., Catalog #MA5-11338); MerTk (e.g., DS5MMER, Catalog #12-5751-82); MFSD13A; hTERT; gp100; Fas-related antigen 1; a metalloproteinase; Mincle (e.g., OTI2A8, Catalog #TA505101); NA17; NY-ESO-1 (e.g., E978m, Catalog #35-6200); polysialic acid (see, e.g., Watzlawik et al. J Nat Sci. 2015; 1(8):e141); PR1; Sarcoma translocation breakpoints; SLC10A2 (e.g., ThermoFisher Catalog #PA5-18990); SLC17A2 (e.g., ThermoFisher Catalog #PA5-106752); SLC39A5 (e.g., ThermoFisher Catalog #MA5-27260); SLC6A15 (e.g., ThermoFisher Catalog #PA5-52586); SLC6A6 (e.g., ThermoFisher Catalog #PA5-53431); SLC7A5; and CALCR (see, e.g., International Publication No. WO 2015077826).

In some embodiments, an antibody can bind specifically to an antigen associated with anemia. A non-limiting example of an antibody that binds specifically to an antigen associated with anemia includes CD163 (e.g., TBI 304H).

In some embodiments, an antibody can bind specifically to an antigen associated with a viral infection. Non-limiting examples of target antigens and associated antibodies that binds specifically to an antigen associated with a viral infection include DCSIGN (see, e.g., International Publication No. WO2018134389) IFNAR1 (e.g., faralimomab); ASCT2 (e.g., idactamab); ULBP1/2/3/4/5/6 (e.g., PA5-82302); and CLDN1 (e.g., INSERM anti-Claudin-1).

In some embodiments, an antibody can bind specifically to an antigen associated with an autoimmune disease. Non-limiting examples of target antigens and associated antibodies that bind specifically to an antigen associated with an autoimmune disease include CLDN2 (see, e.g., International Publication No. WO 2018123949); IL-21R (e.g., PF-05230900); DCIR; DCLK1 (see, e.g., WO2018222675); Dectin1 (see, e.g., U.S. Pat. No. 9,045,542); GITR (e.g., ragifilimab); ITGAV (e.g., abituzumab); LY9 (e.g., PA5-95601); MICA (e.g., 1E2C8, Catalog #66384-1-IG); MICB (e.g., Catalog #MA5-29422); NOX1 (e.g., Catalog #PA5-103220); CD2 (e.g., BTI-322; siplizumab); CD247 (e.g., AFM15); CD25 (e.g., basiliximab); CD28 (e.g., REGN5668); CD3 (e.g., otelixizumab; visilizumab); CD38 (e.g., felzartamab; AMG 424); CD3E (e.g., foralumab; teplizumab); and CD5 (e.g., MAT 304; zolimomab aritox).

In some embodiments, the antibody is a non-targeted antibody, for example, a non-binding or control antibody.

In some embodiments, the antigen is CD30. In some embodiments, the antibody is an antibody or antigen-binding fragment that binds to CD30, such as described in International Patent Publication No. WO 02/43661. In some embodiments, the anti-CD30 antibody is cAC10, which is described in International Patent Publication No. WO 02/43661. cAC10 is also known as brentuximab. In some embodiments, the anti-CD30 antibody comprises the CDRs of cAC10. In some embodiments, the CDRs are as defined by the Kabat numbering scheme. In some embodiments, the CDRs are as defined by the Chothia numbering scheme. In some embodiments, the CDRs are as defined by the IMGT numbering scheme. In some embodiments, the CDRs are as defined by the AbM numbering scheme. In some embodiments, the anti-CD30 antibody comprises CDR-H1, CDR-H2, CDR-H3, CDR-L1, CDR-L2, and CDR-L3 comprising the amino acid sequences of SEQ ID NOs: 1, 2, 3, 4, 5, and 6, respectively. In some embodiments, the anti-CD30 antibody comprises a heavy chain variable region comprising an amino acid sequence that is at least 95%, at least 96%, at least 97%, at least 98%, at least 99%, or 100% identical to the amino acid sequence of SEQ ID NO: 7 and a light chain variable region comprising an amino acid sequence that is at least 95% at least 96%, at least 97%, at least 98%, at least 99%, or 100% identical to the amino acid sequence of SEQ ID NO: 8. In some embodiments, the anti-CD30 antibody comprises a heavy chain comprising the amino acid sequence of SEQ ID NO: 9 or SEQ ID NO: 10 and a light chain comprising the amino acid sequence of SEQ ID NO: 11.

In some embodiments, an antibody provided herein binds to EphA2. In some embodiments, the antibody comprises CDR-H1, CDR-H2, CDR-H3, CDR-L1, CDR-L2, and CDR-L3 comprising the amino acid sequences of SEQ ID NOs: 12, 13, 14, 15, 16, and 17, respectively. In some embodiments, the anti-EphA2 antibody comprises a heavy chain variable region comprising an amino acid sequence that is at least 95%, at least 96%, at least 97%, at least 98%, at least 99%, or 100% identical to the amino acid sequence of SEQ ID NO: 18 and a light chain variable region comprising an amino acid sequence that is at least 95% at least 96%, at least 97%, at least 98%, at least 99%, or 100% identical to the amino acid sequence of SEQ ID NO: 19. In some embodiments, the anti-EphA2 antibody comprises a heavy chain comprising the amino acid sequence of SEQ ID NO: 20 or SEQ ID NO: 21 and a light chain comprising the amino acid sequence of SEQ ID NO: 22. In some embodiments, the anti-EphA2 antibody comprises a heavy chain comprising the amino acid sequence of SEQ ID NO: 23 or SEQ ID NO: 24 and a light chain comprising the amino acid sequence of SEQ ID NO: 25. In some embodiments, the anti-EphA2 antibody comprises a heavy chain comprising the amino acid sequence of SEQ ID NO: 26 or SEQ ID NO: 27 and a light chain comprising the amino acid sequence of SEQ ID NO: 28. In some embodiments, the antibody is h1C1 or 1C1.

TABLE of
Sequences

| SEQ | | |
|---|---|---|
| DD | | |
| NO | Description | Sequence |
| 1 | cAC10 CDR-H1 | DYYIT |
| 2 | cAC10 CDR-H2 | WIYPGSGNTKYNEKFKG |
| 3 | CAC10 CDR-H3 | YGNYWFAY |
| 4 | CAC10 CDR-L1 | KASQSVDFDGDSYMN |
| 5 | cAC10 CDR-L2 | AASNLES |
| 6 | CAC10 CDR-L3 | QQSNEDPWT |
| 7 | CAC10 VH | QIQLQQSGPEVVKPGASVKISCKASGYTFTDYYITWVKQKPGQGLEWIGWIYPGSGNTKYNE |
| | | KFKGKATLTVDTSSSTAFMQLSSLTSEDTAVYFCANYGNYWFAYWGQGTQVTVSA |
| 8 | CAC10 VL | DIVLTQSPASLAVSLGQRATISCKASQSVDFDGDSYMNWYQQKPGQPPKVLIYAASNLESGIP |
| | | ARFSGSGSGTDFTLNIHPVEEEDAATYYCQQSNEDPWTFGGGTKLEIK |
| 9 | CAC10 HC | QIQLQQSGPEVVKPGASVKISCKASGYTFTDYYITWVKQKPGQGLEWIGWIYPGSGNTKYNE |
| | | KFKGKATLTVDTSSSTAFMQLSSLTSEDTAVYFCANYGNYWFAYWGQGTQVTVSAAST |
| | | KGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 10 | CAC10 HC v2 | QIQLQQSGPEVVKPGASVKISCKASGYTFTDYYITWVKQKPGQGLEWIGWIYPGSGNTKYNE |
| | | KFKGKATLTVDTSSSTAFMQLSSLTSEDTAVYFCANYGNYWFAYWGQGTQVTVSAAST |
| | | KGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPG |
| 11 | CAC10 LC | DIVLTQSPASLAVSLGQRATISCKASQSVDFDGDSYMNWYQQKPGQPPKVLIYAASNLES |
| | | GIPARFSGSGSGTDFTLNIHPVEEEDAATYYCQQSNEDPWTFGGGTKLEIKR |
| | | TVAAPSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |
| 12 | h1C1 CDR-H1 | HYMMA |
| 13 | h1C1 CDR-H2 | RIGPSGGPTHYADSVKG |
| 14 | h1C1 CDR-H3 | YDSGYDYVAVAGPAEYFQH |
| 15 | h1C1 CDR-L1 | RASQSISTWLA |
| 16 | h1C1 CDR-L2 | KASNLHT |
| 17 | h1C1 CDR-L3 | QQYNSYSRT |
| 18 | h1C1 VH | EVQLLESGGGLVQPGGSLRLSCAASGFTFSHYMMAWVRQAPGKGLEWVSRIGPSGGPTHYA |
| | | DSVKGRFTISRDNSKNTLYLQMNSLRAEDTAVYYCAGYDSGYDYVAVAGPAEYFQHWGQG |
| | | TLVTVSS |
| 19 | h1C1 VL | DIQMTQSPSSLSASVGDRVTITCRASQSISTWLAWYQQKPGKAPKLLIYKASNLHTGVPSRFSG |
| | | SGSGTEFSLTISGLQPDDFATYYCQQYNSYSRTFGQGTKVEIK |
| 20 | h1C1 HC | EVQLLESGGGLVQPGGSLRLSCAASGFTFSHYMMAWVRQAPGKGLEWVSRIGPSGGPTHYA |
| | | DSVKGRFTISRDNSKNTLYLQMNSLRAEDTAVYYCAGYDSGYDYVAVAGPAEYFQHWGQG |
| | | TLVTVSSASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 21 | h1C1 HC v2 | EVQLLESGGGLVQPGGSLRLSCAASGFTFSHYMMAWVRQAPGKGLEWVSRIGPSGGPTHYA |
| | | DSVKGRFTISRDNSKNTLYLQMNSLRAEDTAVYYCAGYDSGYDYVAVAGPAEYFQHWGQG |
| | | TLVTVSSASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPG |
| 22 | h1C1 LC | DIQMTQSPSSLSASVGDRVTITCRASQSISTWLAWYQQKPGKAPKLLIYKASNLHTGVPSRFSG |
| | | SGSGTEFSLTISGLQPDDFATYYCQQYNSYSRTFGQGTKVEIKRTVAAPSVFIFPPSDEQLKSGT |
| | | ASVVCLLNNFYPREAKVQWKVDNALQSGNSQESVTEQDS |
| | | KDSTYSLSSTLTLSKADYEKHKVYACEVTHQGLSSPVTKSFNRGEC |
| 23 | h1C1 mIgG2a HC | EVQLLESGGGLVQPGGSLRLSCAASGFTFSHYMMAWVRQAPGKGLEWVSRIGPSGGPTHYA |
| | | DSVKGRFTISRDNSKNTLYLQMNSLRAEDTAVYYCAGYDSGYDYVAVAGPAEYFQHWGQG |
| | | TLVTVSSAKTTAPSVYPLAPVCGDTTGSSVTLGCLVKGYFPEPVTLTWNSGSLSSGVHTFPAV |
| | | LQSDLYTLSSSVTVTSSTWPSQSITCNVAHPASSTKVDKKIEPRGPTIKPCPPCKCPAPNLLGGP |
| | | SVFIFPPKIKDVLMISLSPIVTCVVVDVSEDDPDVQISWFVNNVEVHTAQTQTHREDYNSTLRV |
| | | VSALPIQHQDWMSGKEFKCKVNNKDLPAPIERTISKPKGSVRAPQVYVLPPPEEEMTKKQVTL |
| | | TCMVTDFMPEDIYVEWTNNGKTELNYKNTEPVLDSDGSYFMYSKLRVEKKNWVERNSYSCS |
| | | VVHEGLHNHHTTKSFSRTPGK |
| 24 | h1C1 mIgG2a HC | EVQLLESGGGLVQPGGSLRLSCAASGFTFSHYMMAWVRQAPGKGLEWVSRIGPSGGPTHYA |
| | v2 | DSVKGRFTISRDNSKNTLYLQMNSLRAEDTAVYYCAGYDSGYDYVAVAGPAEYFQHWGQG |
| | | TLVTVSSAKTTAPSVYPLAPVCGDTTGSSVTLGCLVKGYFPEPVTLTWNSGSLSSGVHTFPAV |
| | | LQSDLYTLSSSVTVTSSTWPSQSITCNVAHPASSTKVDKKIEPRGPTIKPCPPCKCPAPNLLGGP |
| | | SVFIFPPKIKDVLMISLSPIVTCVVVDVSEDDPDVQISWFVNNVEVHTAQTQTHREDYNSTLRV |
| | | VSALPIQHQDWMSGKEFKCKVNNKDLPAPIERTISKPKGSVRAPQVYVLPPPEEEMTKKQVTL |
| | | TCMVTDFMPEDIYVEWTNNGKTELNYKNTEPVLDSDGSYFMYSKLRVEKKNWVERNSYSCS |
| | | VVHEGLHNHHTTKSFSRTPG |
| 25 | h1C1 mIgG2a LC | DIQMTQSPSSLSASVGDRVTITCRASQSISTWLAWYQQKPGKAPKLLIYKASNLHTGVPSRFSG |
| | | SGSGTEFSLTISGLQPDDFATYYCQQYNSYSRTFGQGTKVEIKRADAAPTVSIFPPSSEQLTSGG |
| | | ASVVCFLNNFYPKDINVKWKIDGSERQNGVLNSWTDQDSKDSTYSMSSTLTLTKDEYERHNS |
| | | YTCEATHKTSTSPIVKSFNRNEC |
| 26 | h1C1 mIgG2a | EVQLLESGGGLVQPGGSLRLSCAASGFTFSHYMMAWVRQAPGKGLEWVSRIGPSGGPTHYA |
| | LALAPG HC | DSVKGRFTISRDNSKNTLYLQMNSLRAEDTAVYYCAGYDSGYDYVAVAGPAEYFQHWGQG |
| | | TLVTVSSAKTTAPSVYPLAPVCGDTTGSSVILGCLVKGYFPEPVTLTWNSGSLSSGVHTFPAV |
| | | LQSDLYTLSSSVTVTSSTWPSQSITCNVAHPASSTKVDKKIEPRGPTIKPCPPCKCPAPNAAGGP |
| | | SVFIFPPKIKDVLMISLSPIVTCVVVDVSEDDPDVQISWFVNNVEVHTAQTQTHREDYNSTLRV |
| | | VSALPIQHQDWMSGKEFKCKVNNKDLGAPIERTISKPKGSVRAPQVYVLPPPEEEMTKKQVT |
| | | LTCMVTDFMPEDIYVEWTNNGKTELNYKNTEPVLDSDGSYFMYSKLRVEKKNWVERNSYSC |
| | | SVVHEGLHNHHTTKSFSRTPGK |
| 27 | h1C1 mIgG2a | EVQLLESGGGLVQPGGSLRLSCAASGFTFSHYMMAWVRQAPGKGLEWVSRIGPSGGPTHYA |
| | LALAPG HC v2 | DSVKGRFTISRDNSKNTLYLQMNSLRAEDTAVYYCAGYDSGYDYVAVAGPAEYFQHWGQG |
| | | TLVTVSSAKTTAPSVYPLAPVCGDTTGSSVTLGCLVKGYFPEPVTLTWNSGSLSSGVHTFPAV |
| | | LQSDLYTLSSSVTVTSSTWPSQSITCNVAHPASSTKVDKKIEPRGPTIKPCPPCKCPAPNAAGGP |
| | | SVFIFPPKIKDVLMISLSPIVTCVVVDVSEDDPDVQISWFVNNVEVHTAQTQTHREDYNSTLRV |
| | | VSALPIQHQDWMSGKEFKCKVNNKDLGAPIERTISKPKGSVRAPQVYVLPPPEEEMTKKQVT |
| | | LTCMVTDFMPEDIYVEWTNNGKTELNYKNTEPVLDSDGSYFMYSKLRVEKKNWVERNSYSC |
| | | SVVHEGLHNHHTTKSFSRTPG |
| 28 | h1C1 mIgG2a | DIQMTQSPSSLSASVGDRVTITCRASQSISTWLAWYQQKPGKAPKLLIYKASNLHTGVPSRFSG |
| | LALAPGLC | SGSGTEFSLTISGLQPDDFATYYCQQYNSYSRTFGQGTKVEIKRADAAPTVSIFPPSSEQLTSGG |
| | | ASVVCFLNNFYPKDINVKWKIDGSERQNGVLNSWTDQDSKDSTYSMSSTLTLTKDEYERHNS |
| | | YTCEATHKTSTSPIVKSFNRNEC |

### Compounds of Formula (I)

Some embodiments provide compounds of Formula (II):

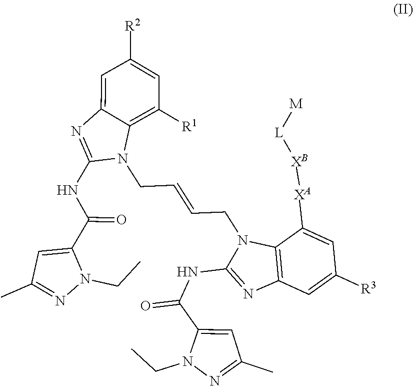

or a pharmaceutically acceptable salt thereof, wherein:
M is a succinimide or a hydrolyzed succinimide;
R¹ is hydrogen, hydroxyl, C₁₋₆ alkoxy, —(C₁₋₆ alkyl)C₁₋₆ alkoxy, —(CH₂)ₙ—NR^{A}R^{B}, or PEG2 to PEG4;
each R² and R³ are independently —CO₂H, —(C═O)ₘ—NR^{C}R^{D}, or —(CH₂)_{q}—NR^{E}R^{F};
each R^{A}, R^{B}, R^{C}, R^{D}, R^{E}, and R^{F} are independently hydrogen or C₁₋₃ alkyl;
each subscript n is independently an integer from 0 to 6;
each subscript m is independently 0 or 1;
each subscript q is independently an integer from 0 to 6;
X^{A} is —CH₂—, —O—, —S—, —NH—, or —N(CH₃)—;
X^{B} is absent or a 2-16 membered heteroalkylene;
X^{B}, M, and L are each independently optionally substituted with a PEG Unit from
PEG1 to PEG 72; and
L is an optional linker as described herein.

In some embodiments, the compound of Formula (II) has the structure:

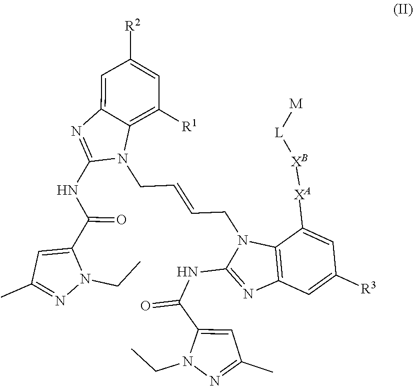

or a pharmaceutically acceptable salt thereof, wherein:
R¹ is hydrogen, hydroxyl, C₁₋₆ alkoxy, —(C₁₋₆ alkyl)C₁₋₆ alkoxy, —(CH₂)ₙ—NR^{A}R^{B}, or PEG2 to PEG4;
each R² and R³ are independently —CO₂H, —(C═O)ₘ—NR^{C}R^{D}, or —(CH₂)_{q}—NR^{E}R^{F};
each R^{A}, R^{B}, R^{C}, R^{D}, R^{E}, and R^{F} are independently hydrogen or C₁₋₃ alkyl;
each subscript n is independently an integer from 0 to 6;
each subscript m is independently 0 or 1;
each subscript q is independently an integer from 0 to 6;
X^{A} is —CH₂—, —O—, —S—, —NH—, or —N(CH₃)—;
X^{B} is absent or 2-16 membered heteroalkylene;
L is a linker having the formula -(A)ₐ-(W)_{w}-(Y)_{y}—, wherein:
A is a C₂₋₂₀ alkylene optionally substituted with 1-3 R^{a1}; or a 2 to 40 membered heteroalkylene optionally substituted with 1-3 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —C(O)NR^{d1}R^{e1}, —C(O)(C₁₋₆ alkyl), and —C(O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —C(O)NR^{d1}R^{e1}, —C(O)(C₁₋₆ alkyl), and —C(O)O(C₁₋₆ alkyl);
each R^{d1} and R^{e1} are independently hydrogen or C₁₋₃ alkyl;
W is from 1-12 amino acids or has the structure:

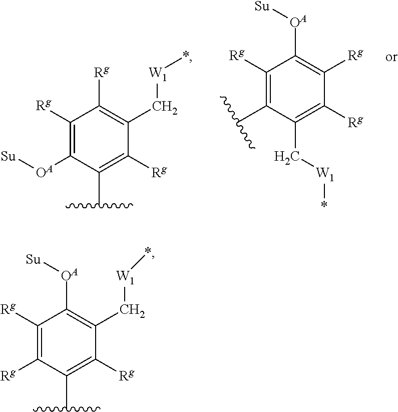

wherein Su is a Sugar moiety;
—O^{A}— represents a glycosidic bond;
each R^{g} is independently hydrogen, halogen, —CN, or —NO₂;
W¹ is absent or —O—C(═O)—;
[symbol] represents covalent attachment to A or M; and * represents covalent attachment to Y, X^{A}, or X^{B}.
Y is a self-immolative moiety, a non-self-immolative releasable moiety, or a non-cleavable moiety;
subscript a is 0 or 1;
subscript y is 0 or 1;
subscript w is 0 or 1;
M is

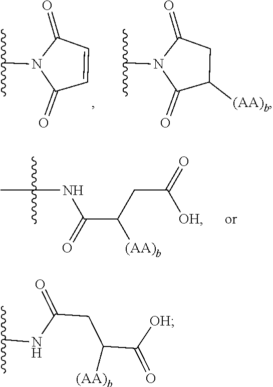

each AA is an independently selected amino acid, wherein (AA)_{b} is connected to the succinimide or hydrolyzed succinimide via a sulfur atom;
each subscript b is independently an integer from 1 to 6; and
X^{B} and L are each independently optionally substituted with a PEG Unit from PEG2 to PEG 72.

As used herein, A, when present is covalently attached to M or M¹, and Y, when present is attached to X^{B} or to X^{A} (when X^{B} is absent).

In some embodiments, M is

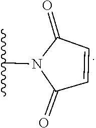 [SMILES: *N1C(=O)C=CC1=O]

In some embodiments, M is

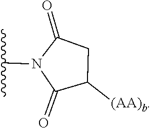

In some aspects, M is

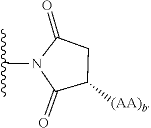

In some aspects, M is

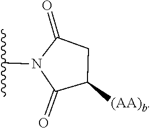

In some embodiments, M is

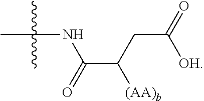

In some aspects, M is

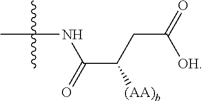

In some aspects, M is

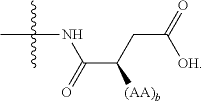

In some embodiments, M is

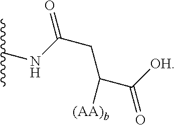

In some aspects, M is

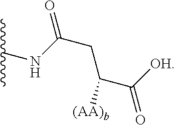

In some aspects, M is

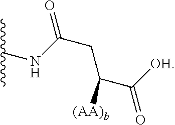

In some embodiments, each AA is independently a natural amino acid; wherein (AA)_{b} is connected to the succinimide or hydrolyzed succinimide via a sulfur atom. In some embodiments, each AA is independently a natural amino acid; wherein (AA)_{b} is connected to the succinimide or hydrolyzed succinimide via a sulfur atom of a cysteine residue.

In some embodiments, each AA is independently a natural amino acid; wherein (AA)_{b} is connected to the succinimide or hydrolyzed succinimide via a nitrogen atom. In some embodiments, each AA is independently a natural amino acid; wherein (AA)_{b} is connected to the succinimide or hydrolyzed succinimide via the c-nitrogen atom of a lysine residue.

In some embodiments, each subscript b is 1, 2, or 3. In some embodiments, each subscript b is 1. In some embodiments, each subscript b is 2. In some embodiments, each subscript b is 3. In some embodiments, each subscript b is 3, 4, 5, or 6. In some embodiments, each subscript b is 4. In some embodiments, each subscript b is 5. In some embodiments, each subscript b is 6.

In some embodiments, M is

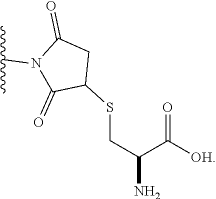 [SMILES: *N1C(=O)CC(SC[C@H](N)C(=O)O)C1=O]

In some aspects, M is

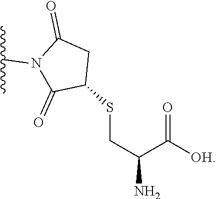 [SMILES: *N1C(=O)C[C@H](SC[C@H](N)C(=O)O)C1=O]

In some aspects, M is

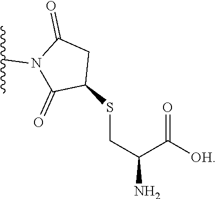 [SMILES: *N1C(=O)C[C@@H](SC[C@H](N)C(=O)O)C1=O]

In some embodiments, M is

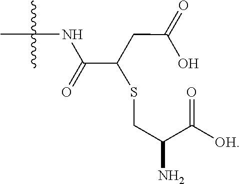 [SMILES: *NC(=O)C(CC(=O)O)SC[C@H](N)C(=O)O]

In some aspects, M is

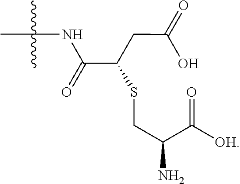 [SMILES: *NC(=O)[C@H](CC(=O)O)SC[C@H](N)C(=O)O]

In some aspects, M is

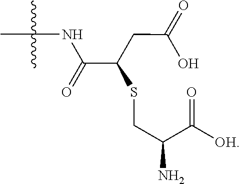 [SMILES: *NC(=O)[C@@H](CC(=O)O)SC[C@H](N)C(=O)O]

In some aspects, M is

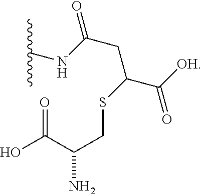 [SMILES: *NC(=O)CC(SC[C@H](N)C(=O)O)C(=O)O]

In some aspects, M is

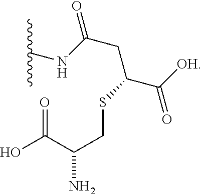 [SMILES: *NC(=O)C[C@@H](SC[C@H](N)C(=O)O)C(=O)O]

In some aspects, M is

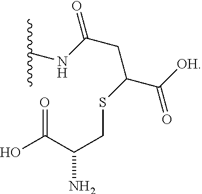 [SMILES: *NC(=O)CC(SC[C@H](N)C(=O)O)C(=O)O]

In some embodiments, R¹ is methoxy and R² and R³ are both —C(═O)NH₂. In some embodiments, X^{A} is —O— and X^{B} is

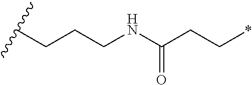

wherein [symbol] represents covalent linkage to X^{A}, and * represents covalent linkage to L, when present, or M. In some embodiments, R¹ is methoxy; R² and R³ are both —C(═O)NH₂; X^{A} is —O—; and X^{B} is

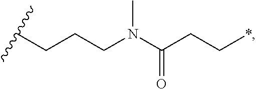

wherein [symbol] represents covalent linkage to X^{A}, and * represents covalent linkage to L, when present, or M. In some such embodiments, L is absent. In some embodiments, R¹ is methoxy; R² and R³ are both —C(═O)NH₂; X^{A} is —O—; X^{B} is

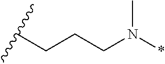

wherein [symbol] represents covalent linkage to X^{A}, and * represents covalent linkage to L; and subscript a and subscript y are both 0 (i.e., X^{B} is covalently attached to W). In some embodiments, X^{A} is —O—; X^{B} is

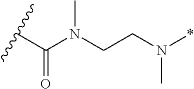

wherein [symbol] represents covalent linkage to X^{A}, and * represents covalent linkage to L. In some embodiments, R¹ is methoxy; R² and R³ are both —C(═O)NH₂; X^{A} is —O—; and X^{B} is

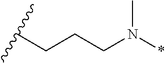

wherein [symbol] represents covalent linkage to X^{A}, and * represents covalent linkage to L; and subscript a and subscript w are both 0.

In some embodiments, R¹ is methoxy; R² and R³ are both —C(═O)NH₂; X^{A} is —O—; and X^{B} is

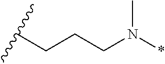

wherein [symbol] represents covalent linkage to X^{A}, and * represents covalent linkage to L; and subscript y and subscript w are both 0.

In some embodiments, R¹ is methoxy; R² and R³ are both —C(═O)NH₂; X^{A} is —O—; and X^{B} is

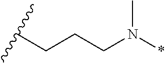

wherein [symbol] represents covalent linkage to X^{A}, and * represents covalent linkage to L; and subscript y is 0.

In some embodiments, R¹ is methoxy and R² and R³ are both —C(═O)NH₂. In some embodiments, X^{A} is —CH₂—; and X^{B} is

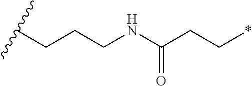

wherein [symbol] represents covalent linkage to X^{A}, and * represents covalent linkage to L, when present, or M. In some embodiments, R¹ is methoxy; R² and R³ are both —C(═O)NH₂; X^{A} is —CH₂—; and X^{B} is

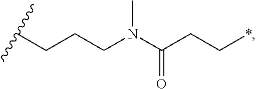

wherein [symbol] represents covalent linkage to X^{A}, and * represents covalent linkage to L, when present, or M. In some embodiments, R¹ is methoxy; R² and R³ are both —C(═O)NH₂; X^{A} is —CH₂—; and X^{B} is

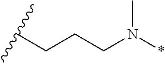

wherein [symbol] represents covalent linkage to X^{A}, and * represents covalent linkage to L; and subscript a and subscript y are both 0 (i.e., X^{B} is covalently attached to W). In some embodiments, X^{A} is —CH₂—; and X^{B} is

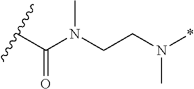

wherein [symbol] represents covalent linkage to X^{A}, and * represents covalent linkage to L. In some embodiments, R¹ is methoxy; R² and R³ are both —C(═O)NH₂; X^{A} is —CH₂—; and X^{B} is

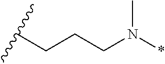

wherein [symbol] represents covalent linkage to X^{A}, and * represents covalent linkage to L; and subscript a and subscript w are both 0 (i.e., X^{B} is covalently bound to Y).

In some such embodiments, L is a linker having the formula -(A)ₐ-(W)_{w}-(Y)_{y}-

In some embodiments: X^{B} is absent and L is covalently attached to X^{A}. In some embodiments: X^{B} is absent and Y is covalently attached to X^{A}. In some embodiments: X^{B} is absent and Y is absent, and W is covalently attached to X^{A}. In some embodiments: X^{B} is absent, Y is absent, W is absent, and A is covalently attached to X^{A}.

In some embodiments: X^{B} is a 2-16 membered heteroalkylene and L is covalently attached to X^{B}. In some embodiments: X^{B} is a 2-16 membered heteroalkylene and Y is covalently attached to X^{B}. In some embodiments: X^{B} is a 2-16 membered heteroalkylene, Y is absent, and W is covalently attached to X^{B}. In some embodiments: X^{B} is a 2-16 membered heteroalkylene, Y is absent, W is absent, and A is covalently attached to X^{B}.

In some embodiments, W₁ is —OC(═O)— and subscript y is 1. In some embodiments, X^{A} is —O— and X^{B} and W are absent. In some embodiments, X^{A} is NH or —O—, X^{B} is absent, and W₁ is —OC(═O). In some embodiments, X^{A} is —N(CH₃)—, X^{B} is absent, and W₁ is —OC(═O). In some embodiments, X^{A} is —S—, X^{B} is absent, and W₁ is —OC(═O). In some embodiments, W₁ is —OC(═O)— and X^{B} is covalently attached to W via —O— or —NH—.

In some embodiments, A is covalently attached to M. In some embodiments, when subscript a is 0 and subscript w is 0, Y is covalently attached to M. In some embodiments, when subscripts a, y, and w, are each 0, X^{B} is covalently attached to M.

In some embodiments, the compound of Formula (II) is selected from the group consisting of:

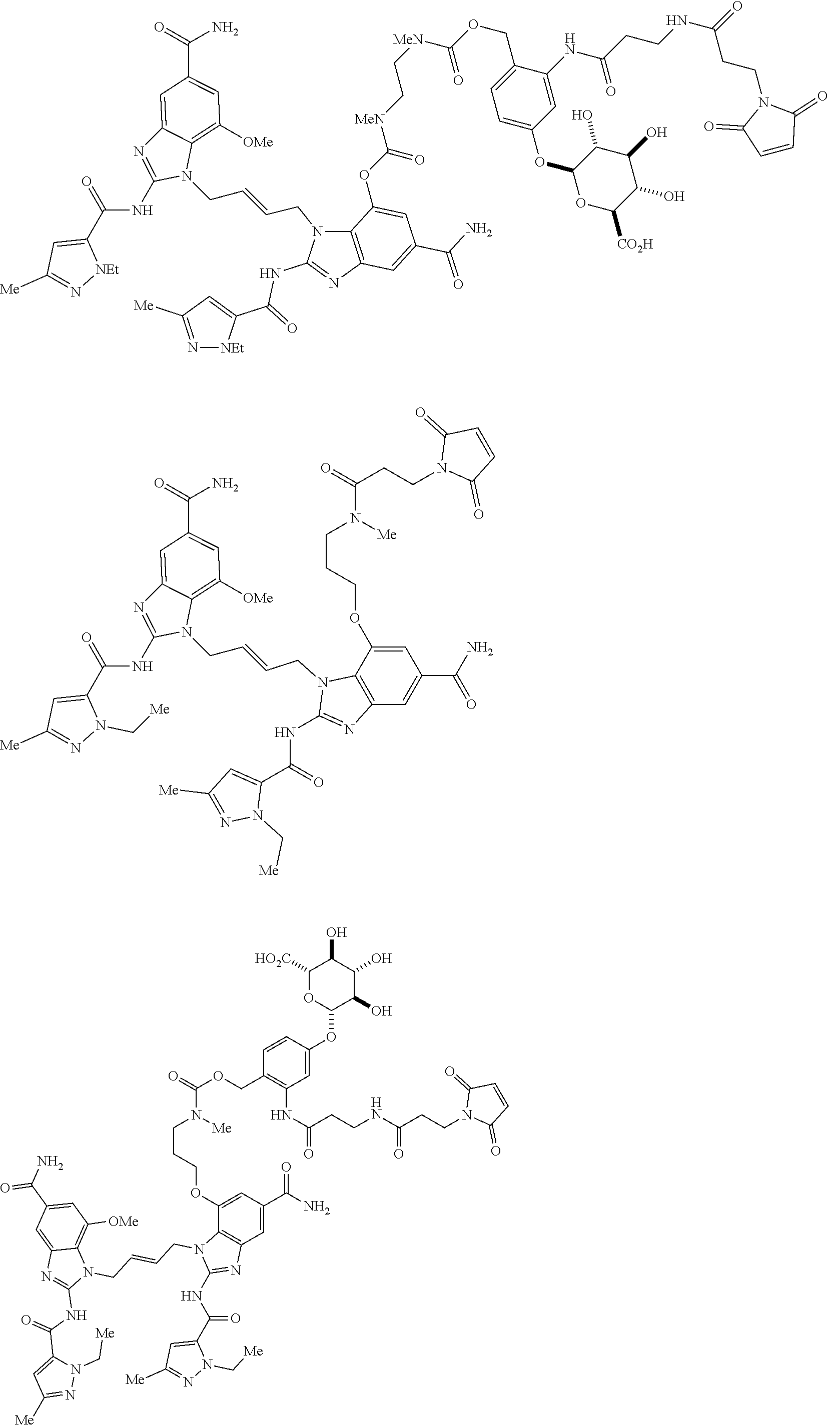

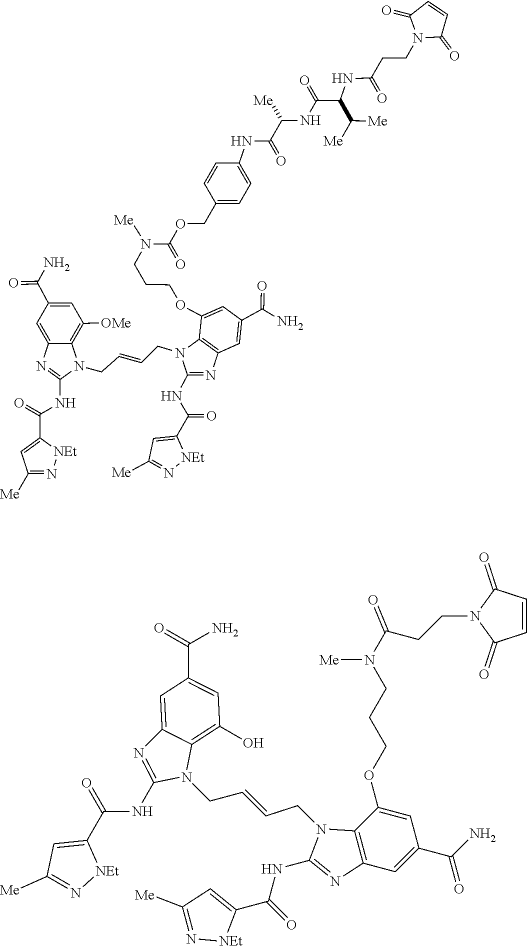

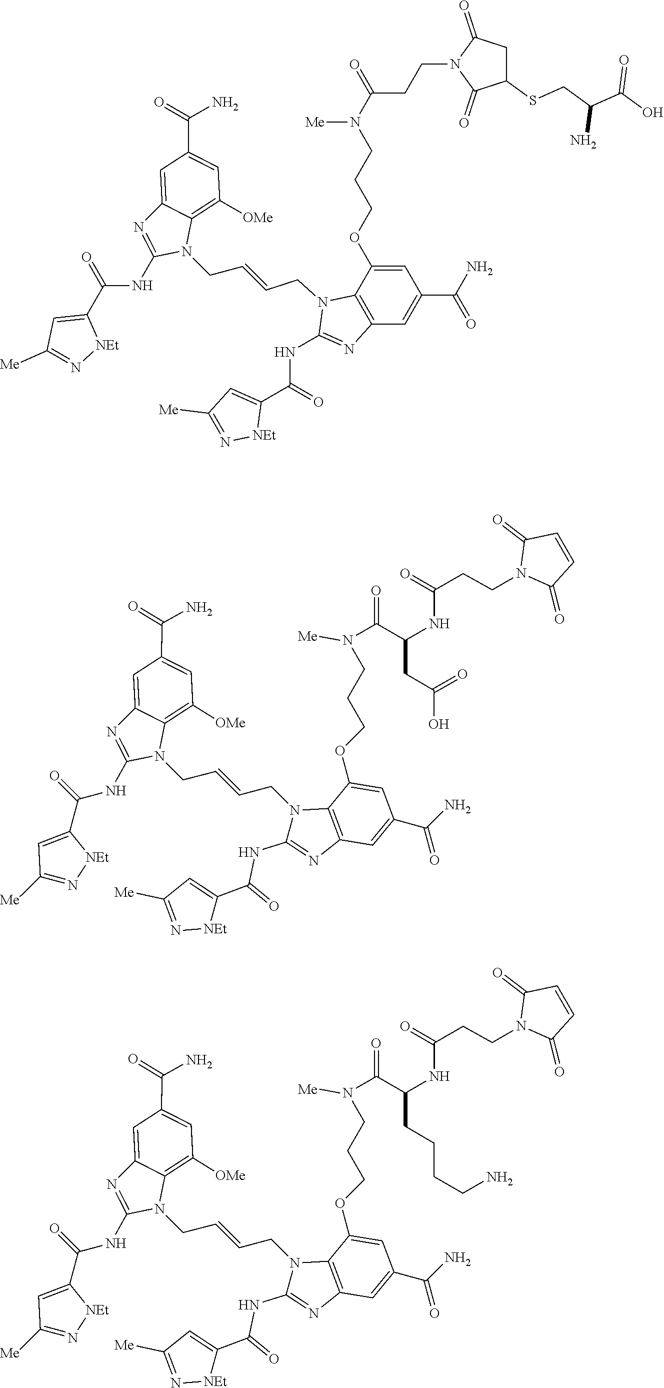

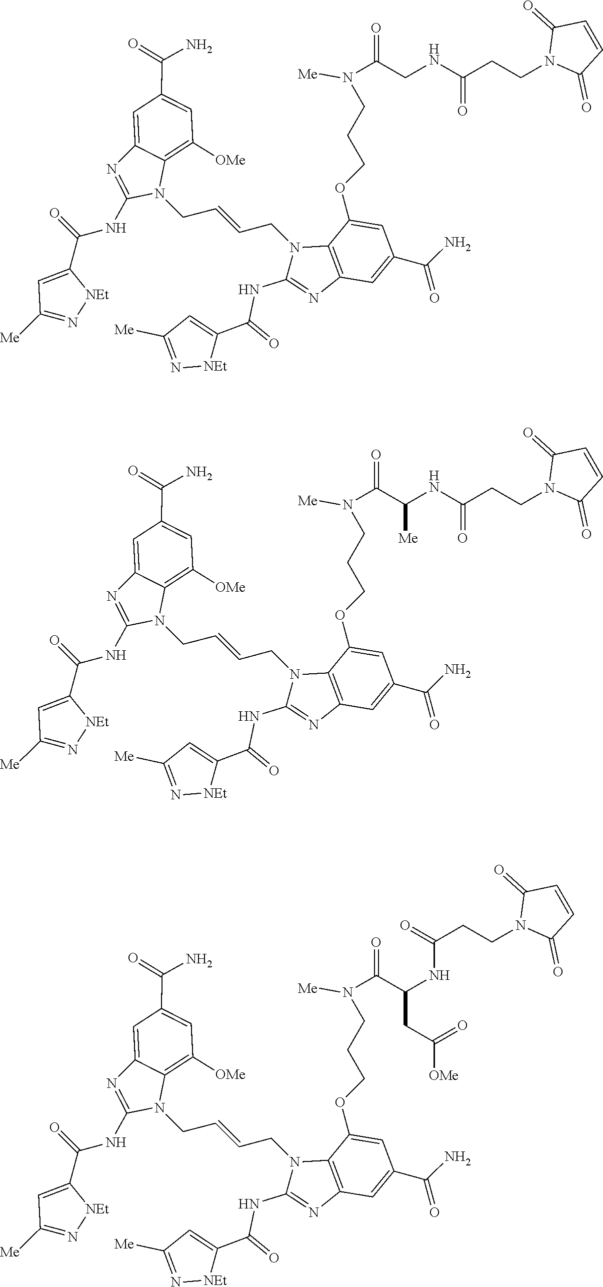

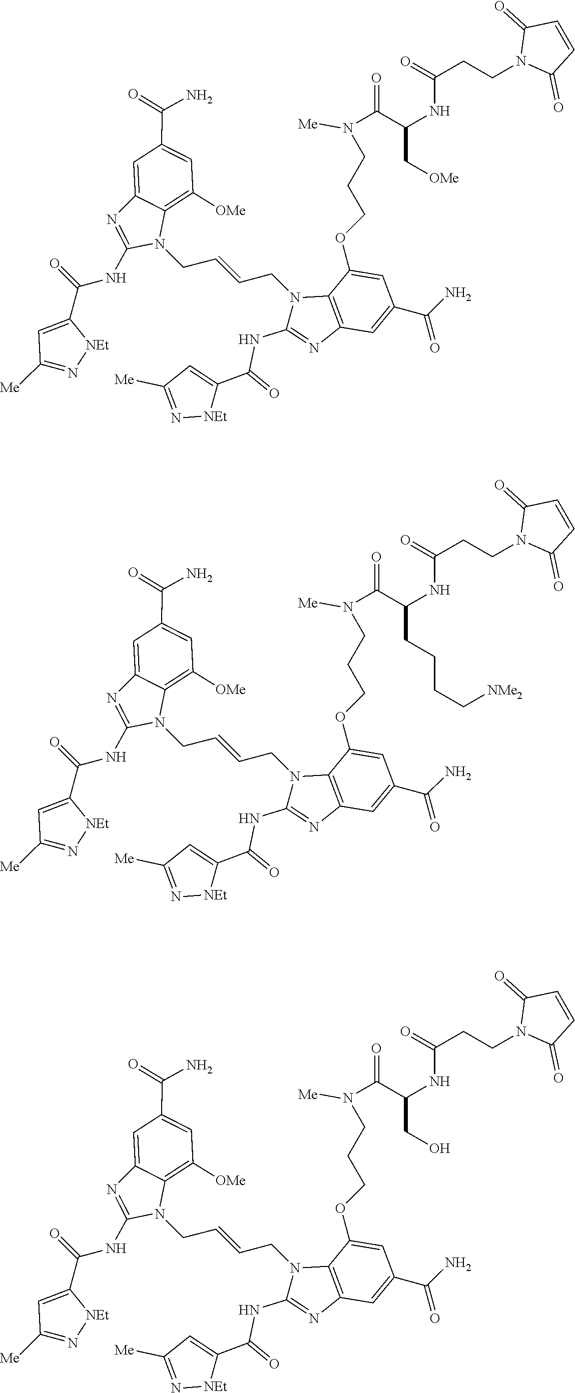

### Compounds of Formula (H-A)

In some embodiments, the compound of Formula (II) has the structure of Formula (II-A):

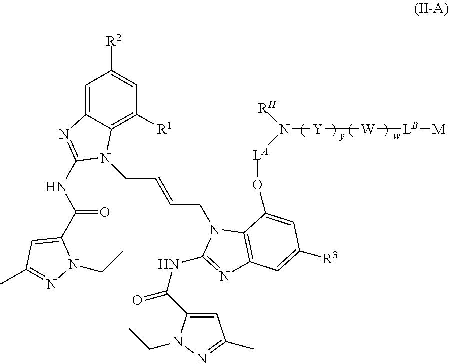

or a pharmaceutically acceptable salt thereof, wherein:
L^{A} is —(CH₂)₁₋₆—, —C(O)(CH₂)₁₋₆—, or —C(O)NR^{H}(CH₂)₁₋₆—;
each R^{H} is independently hydrogen or C₁₋₃ alkyl;
Y is

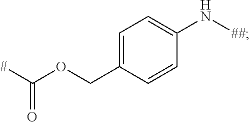

# represents covalent attachment to —NR^{H}L^{A};
## represents covalent attachment to W or L^{B};
L^{B} is —(CH₂)₁₋₆—, —C(O)(CH₂)₁₋₆—, or —[NHC(O)(CH₂)₁₋₄]₁₋₃—; and
the remaining variables are as defined above in connection of Formula (II).

In some embodiments, R^{H} is methyl. In some embodiments, L^{A} is —(CH₂)₂₋₆—. In some embodiments, L^{A} is —(CH₂)₃—. In some embodiments, subscript y is 0. In some embodiments, subscript y is 1. In some embodiments, subscript w is 0. In some embodiments, subscript w is 1. In some embodiments, subscript y and subscript w are both 1. In some embodiments, subscript y and subscript w are both 0. When subscript y and subscript w are both 0, the compound of Formula (II) has the structure of Formula (II-B):

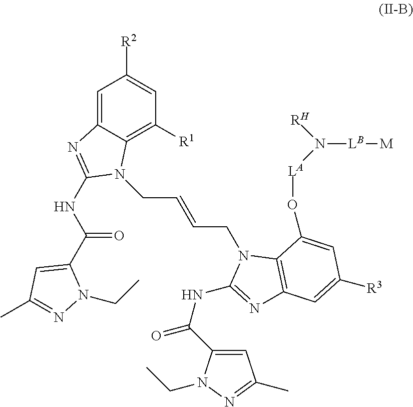

or a pharmaceutically acceptable salt thereof, wherein:
L^{A} is —(CH₂)₁₋₆—, —C(O)(CH₂)₁₋₆—, or —C(O)NR^{H}(CH₂)₁₋₆—;
each R^{H} is independently hydrogen or C₁₋₃ alkyl;
L^{B} is —(CH₂)₁₋₆—, —C(O)(CH₂)₁₋₆—, or —[NHC(O)(CH₂)₁₋₄]₁₋₃—; and
the remaining variables are as defined above in connection of Formula (II).

In some embodiments, W is a chain of 1-6 amino acids. In some embodiments, W is a chain of 1-4 amino acids. In some embodiments, W is a chain of 1-3 amino acids. In some embodiments, each amino acid of W is independently selected from the group consisting of alanine, valine, isoleucine, leucine, aspartic acid, glutamic acid, lysine, histidine, arginine, glycine, serine, threonine, phenylalanine, O-methylserine, O-methylaspartic acid, O-methylglutamic acid, N-methyllysine, O-methyltyrosine, O-methylhistidine, and O-methylthreonine.

In some embodiments, W is:

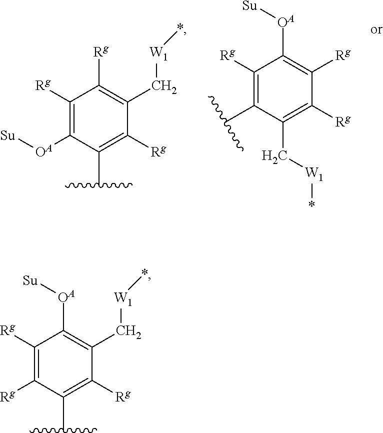

wherein:
[symbol] represents covalent attachment to L^{B}; and
* represents covalent attachment to Y or NR^{H}.

In some embodiments, L^{B} is —C(O)(CH₂)₂—. In some embodiments, L^{B} is —[NHC(O)(CH₂)₂]₂—. In some embodiments, M is

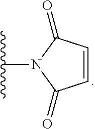 [SMILES: *N1C(=O)C=CC1=O]

In some embodiments, M is

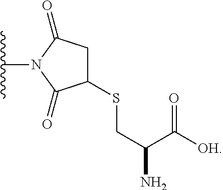 [SMILES: *N1C(=O)CC(SC[C@H](N)C(=O)O)C1=O]

In some aspects, M is

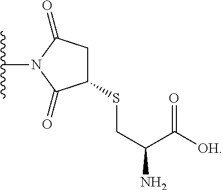 [SMILES: *N1C(=O)C[C@H](SC[C@H](N)C(=O)O)C1=O]

In some aspects, M is

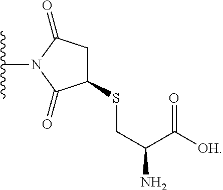 [SMILES: *N1C(=O)C[C@@H](SC[C@H](N)C(=O)O)C1=O]

In some embodiments, the compound of Formula (II-A) is selected from the group consisting of:

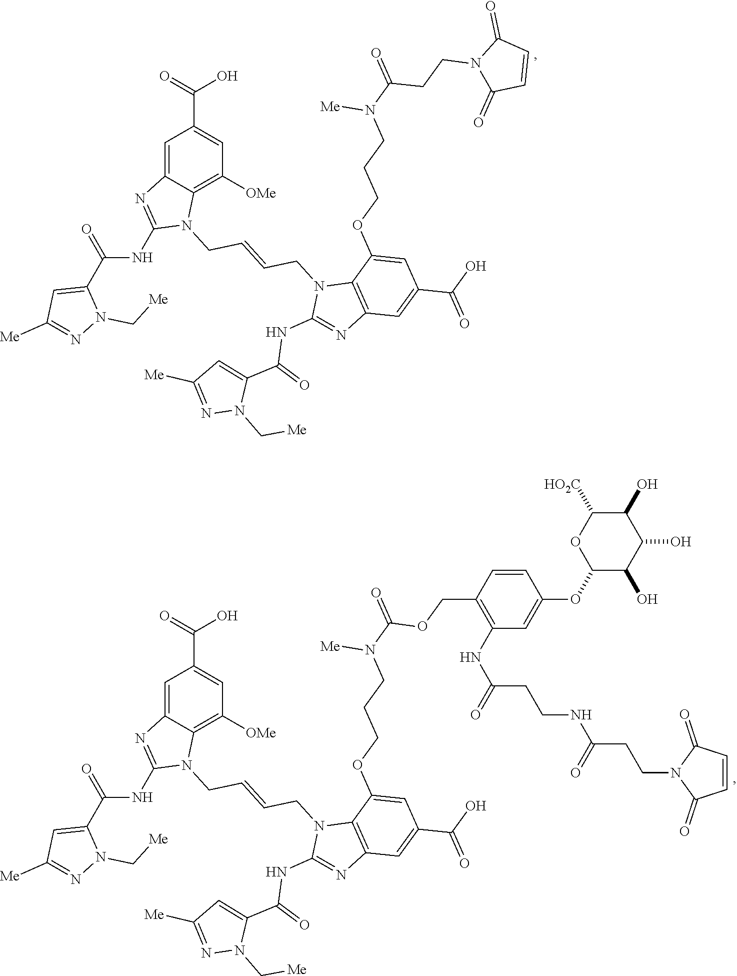

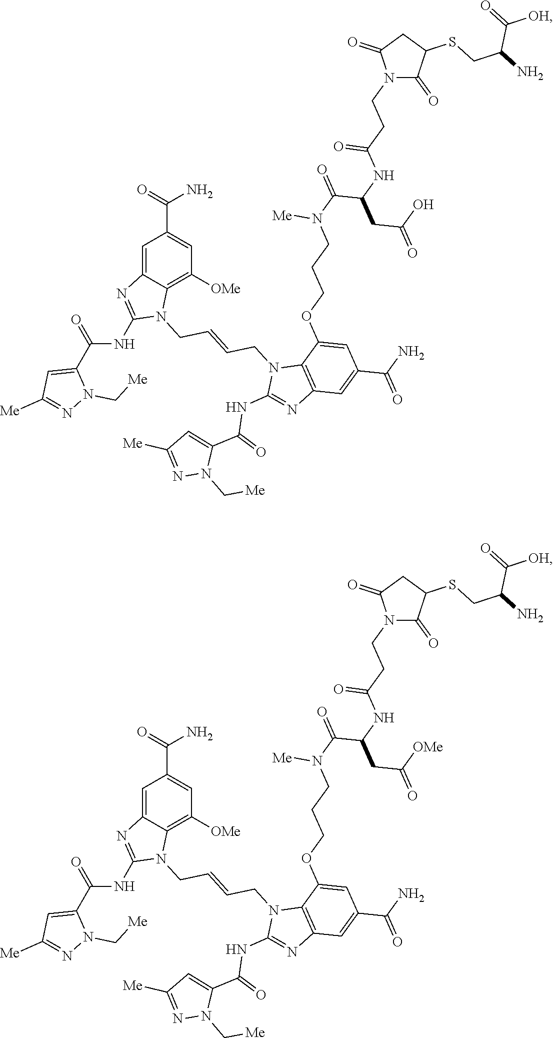 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CC(=O)O)NC(=O)CCN3C(=O)CC(SC[C@H](N)C(=O)O)C3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CC(=O)OC)NC(=O)CCN3C(=O)CC(SC[C@H](N)C(=O)O)C3=O)c21]

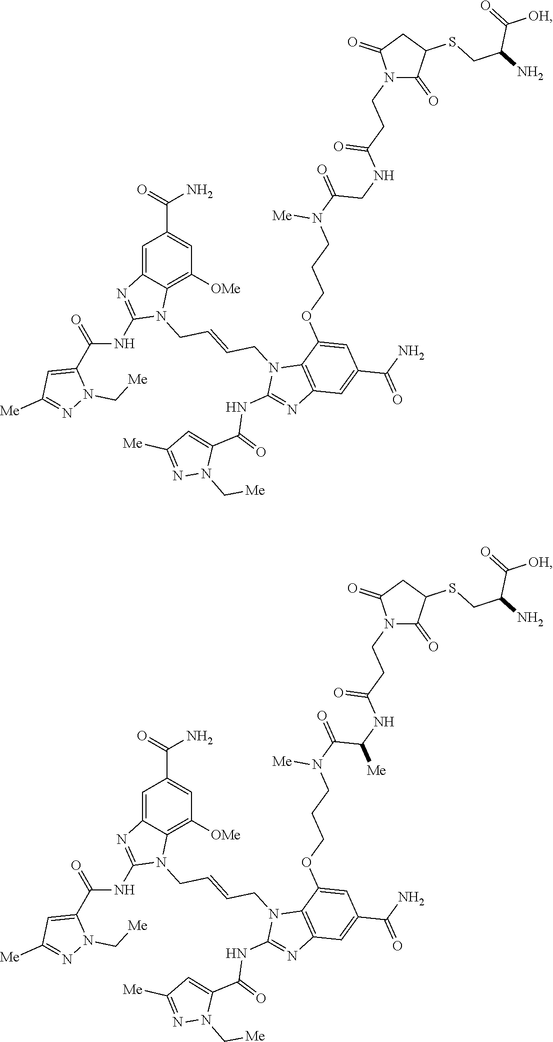 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)CNC(=O)CCN3C(=O)CC(SC[C@H](N)C(=O)O)C3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](C)NC(=O)CCN3C(=O)CC(SC[C@H](N)C(=O)O)C3=O)c21]

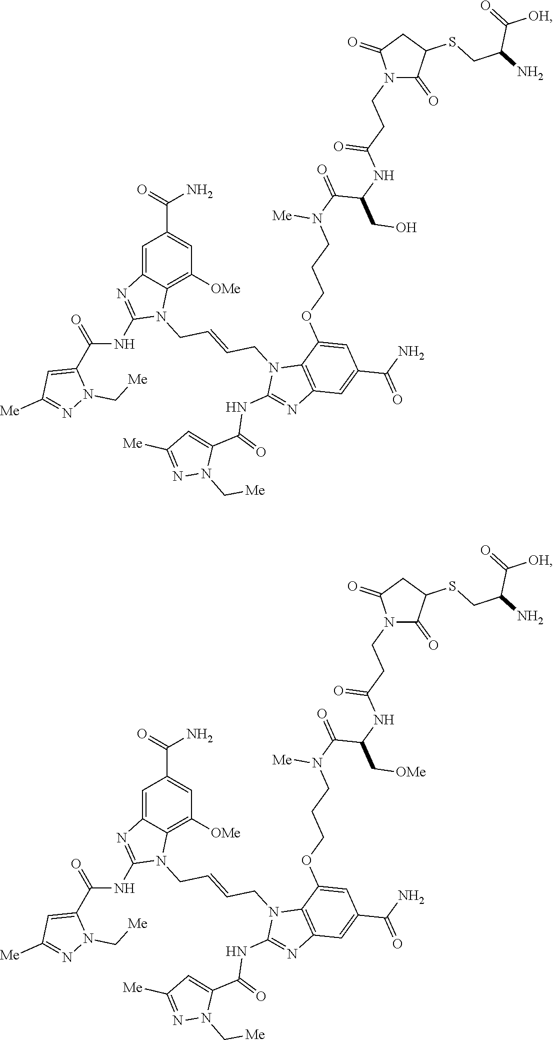 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CO)NC(=O)CCN3C(=O)CC(SC[C@H](N)C(=O)O)C3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](COC)NC(=O)CCN3C(=O)CC(SC[C@H](N)C(=O)O)C3=O)c21]

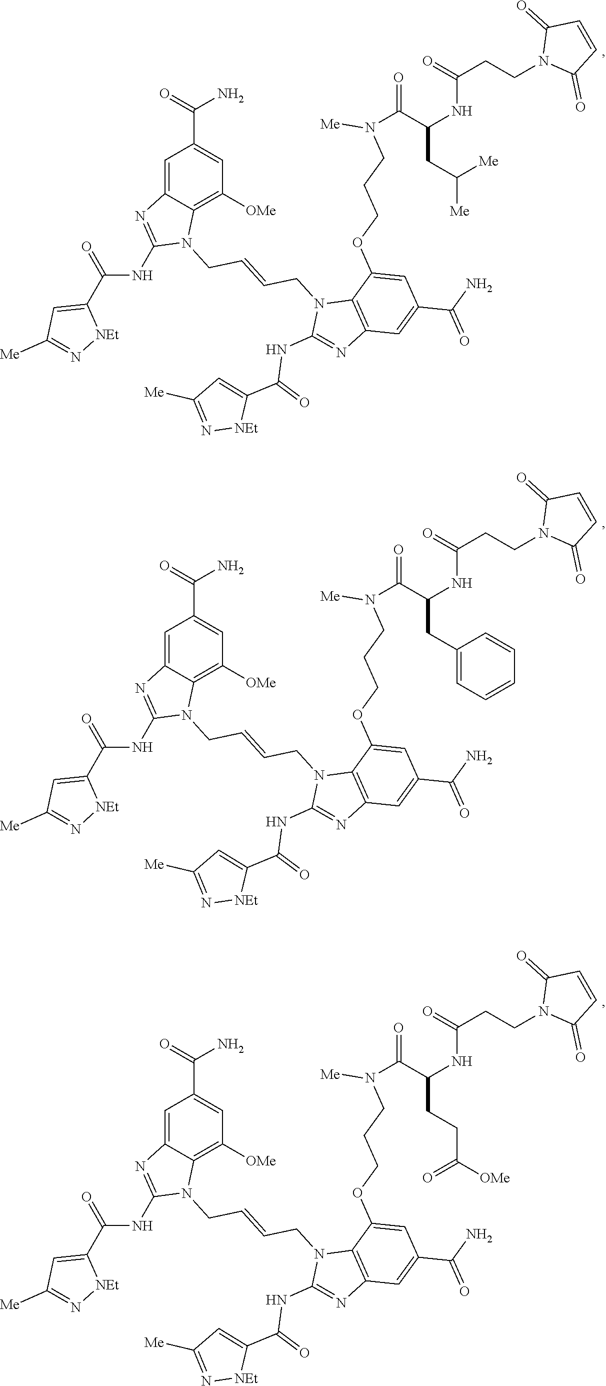

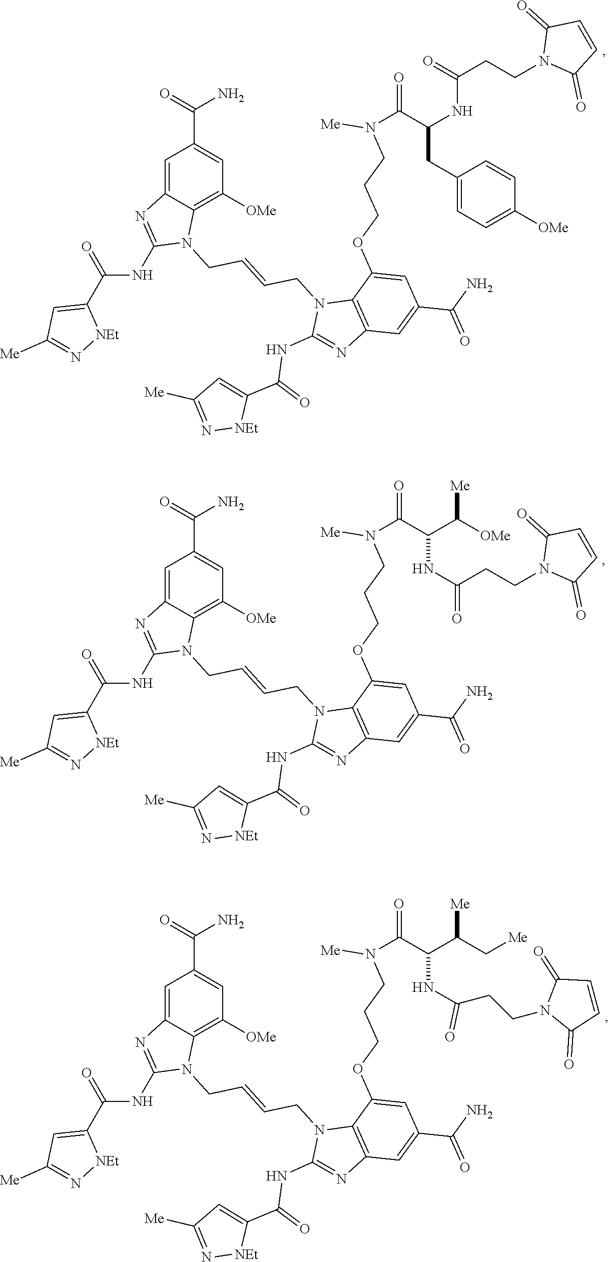

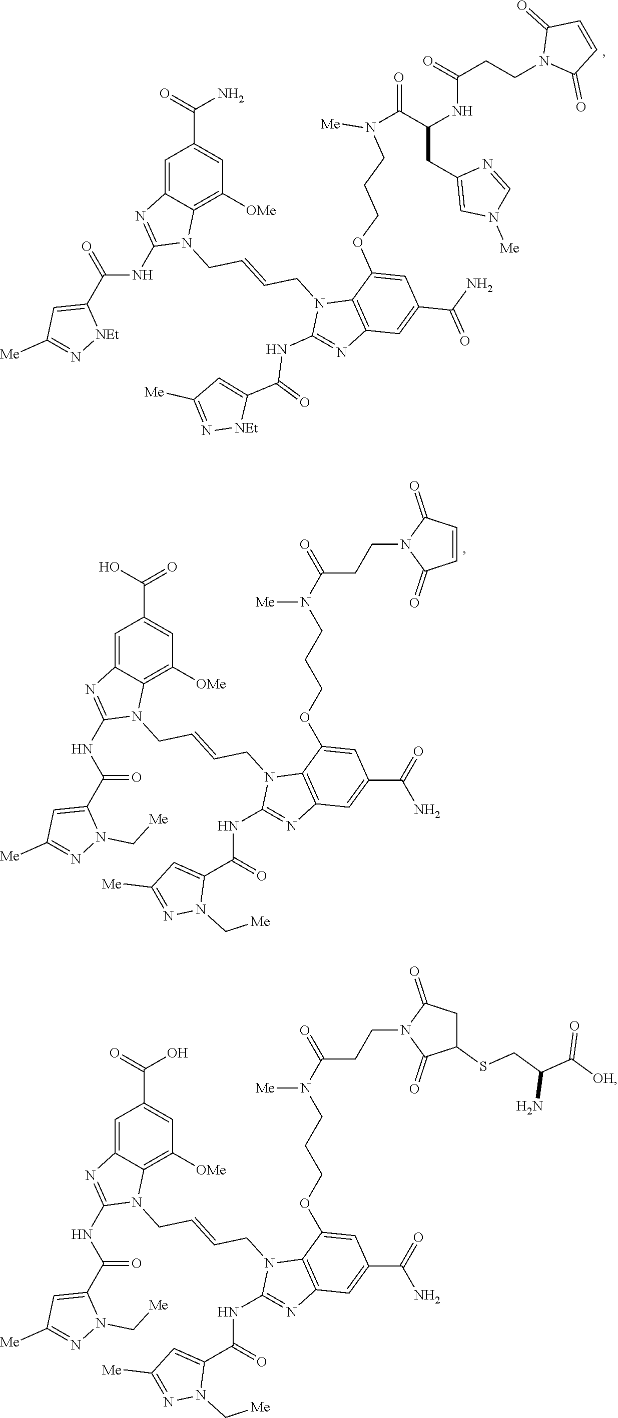

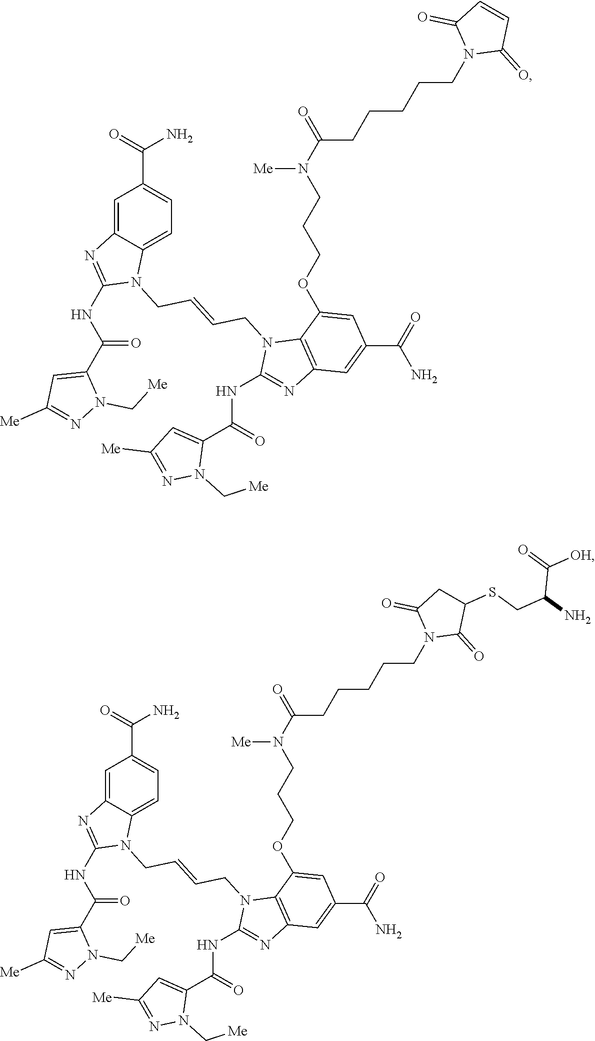 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN(C)C(=O)CCCCCN3C(=O)C=CC3=O)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN(C)C(=O)CCCCCN3C(=O)CC(SC[C@H](N)C(=O)O)C3=O)c21]

and pharmaceutically acceptable salts thereof.

### Compounds of Formula (III)

Some embodiments provide compounds of Formula (III):

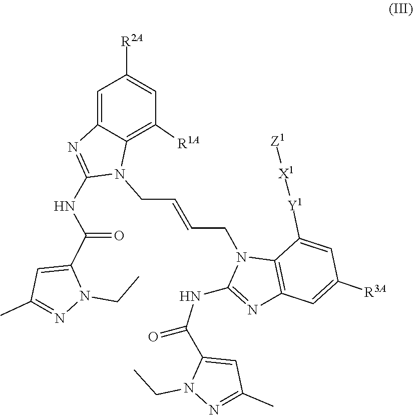

or a pharmaceutically acceptable salt thereof, wherein:
R^{1A} is hydrogen, hydroxyl, C₁₋₆ alkoxy, —(C₁₋₆ alkyl)C₁₋₆ alkoxy, —(CH₂)ₙₙ—NR^{AA}R^{BB}.
each R^{2A} and R^{3A} are independently —CO₂H, —(C═O)ₘₘ—NR^{CC}R^{DD}, or —(CH₂)_{qq}—NR^{EE1}R^{FF1}.
each subscript nn is independently an integer from 0 to 6;
each subscript mm is independently 0 or 1;
each subscript qq is independently an integer from 0 to 6;
Y¹ is —CH₂—, —O—, —S—, —NH—, or —N(CH₃)—;
X¹ is a C₂-C₆ alkylene;
Z¹ is —NR^{EE}R^{FF}, —C(═O)NR^{GG}R^{HH}, or —CO₂H;
each R^{AA}, R^{BB}, R^{CC}, R^{DD}, R^{EE1}, and R^{FF1} are independently hydrogen or C₁₋₃ alkyl; and
each R^{EE}, R^{FF}, R^{GG}, and R^{HH} are independently hydrogen or C₁₋₆ alkyl.

In some embodiments, R^{1A} is hydrogen. In some embodiments, R^{1A} is hydroxyl. In some embodiments, R^{1A} is C₁₋₆ alkoxy. In some embodiments, R¹ is methoxy. In some embodiments, R^{1A} is —(C₁₋₆ alkyl)C₁₋₆ alkoxy. In some embodiments, R^{1A} is methoxyethyl.

In some embodiments, R¹ is —(CH₂)ₙₙ—NR^{AA}R^{BB}. In some embodiments, R^{AA} and R^{BB} are both hydrogen. In some embodiments, R^{AA} and R^{BB} are independently C₁₋₃ alkyl. In some embodiments, one of R^{AA} and R^{BB} is hydrogen and the other of R^{AA} and R^{BB} is C₁₋₃ alkyl. In some embodiments, the C₁₋₃ alkyl is methyl. In some embodiments, each subscript nn is 0. In some embodiments, each subscript nn is 1. In some embodiments, each subscript nn is 2. In some embodiments, each subscript nn is 3. In some embodiments, each subscript nn is 3, 4, 5, or 6. In some embodiments, each subscript nn is 4. In some embodiments, each subscript nn is 5. In some embodiments, each subscript nn is 6.

In some embodiments, each R^{2A} and R^{3A} are independently —CO₂H, —(C═O)ₘₘ—NR^{CC}R^{DD}, or —(CH₂)_{qq}—NR^{EE1}R^{FF1}; and R^{2A} and R^{3A} are the same. In some embodiments, each R^{2A} and R^{3A} are independently —CO₂H, —(C═O)ₘₘ—NR^{CC}R^{DD}, or —(CH₂)_{qq}—NR^{EE1}R^{FF1}; and R^{2A} and R^{3A} are different.

In some embodiments, R^{2A} is —(C═O)ₘₘ—NR^{CC}R^{DD}. In some embodiments, R^{3A} is —(C═O)ₘₘ—NR^{CC}R^{DD}. In some embodiments, each R^{CC} and each R^{DD} is hydrogen. In some embodiments, each R^{CC} and each R^{DD} is independently C₁₋₃ alkyl. In some embodiments, one of each R^{CC} and R^{DD} is hydrogen and the other of each R^{CC} and R^{DD} is C₁₋₃ alkyl. In some embodiments, the C₁₋₃ alkyl is methyl. In some embodiments, each subscript mm is 0. In some embodiments, each subscript mm is 1.

In some embodiments, R^{2A} is —(CH₂)_{qq}—NR^{EE1}R^{FF1}. In some embodiments, R^{3A} is —(CH₂)_{q}—NR^{EE1}R^{FF1}. In some embodiments, each R^{EE1} and each R^{FF1} is hydrogen. In some embodiments, each R^{EE1} and each R^{FF1} is independently C₁₋₃ alkyl. In some embodiments, one of each R^{EE1} and R^{FF1} is hydrogen and the other of each R^{EE1} and R^{FF1} is C₁₋₃ alkyl. In some embodiments, the C₁₋₃ alkyl is methyl. In some embodiments, each subscript q is 0. In some embodiments, each subscript q is an integer from 1 to 6. In some embodiments, each subscript qq is 1. In some embodiments, each subscript qq is 2. In some embodiments, each subscript qq is 3, 4, 5, or 6.

In some embodiments, R^{3A} is —CO₂H. In some embodiments, R^{2A} is —CO₂H.

In some embodiments, Y¹ is —CH₂—. In some embodiments, Y¹ is —O—. In some embodiments, Y¹ is —S—. In some embodiments, Y¹ is —NH—. In some embodiments, Y¹ is —N(CH₃)—.

In some embodiments, X¹ is a C₂-C₅ alkylene. In some embodiments, X¹ is a C₂-C₄ alkylene. In some embodiments, X¹ is ethylene or n-propylene. In some embodiments, X¹ is ethylene. In some embodiments, X¹ is n-propylene.

In some embodiments, Z¹ is —NR^{E1}R^{F1}. In some embodiments, R^{EE} and R^{FF} are both hydrogen. In some embodiments, R^{EE} and R^{FF} are independently C₁₋₆ alkyl. In some embodiments, one of R^{EE} and R^{FF} is hydrogen and the other of R^{EE} and R^{FF} is C₁₋₆ alkyl. In some embodiments, the C₁₋₆ alkyl is a C₁₋₃ alkyl. In some embodiments, the C₁₋₃ alkyl is methyl.

In some embodiments, Z¹ is —C(═O)NR^{GG}R^{HH}. In some embodiments, R^{GG} and R^{HH} are both hydrogen. In some embodiments, R^{GG} and R^{HH} are independently C₁₋₆ alkyl. In some embodiments, one of R^{GG} and R^{HH} is hydrogen and the other of R^{GG} and R^{HH} is C₁₋₆ alkyl. In some embodiments, the C₁₋₆ alkyl is a C₁₋₃ alkyl. In some embodiments, the C₁₋₃ alkyl is methyl. In some embodiments, Z¹ is —CO₂H. In some embodiments, Z¹ is —NR^{EE}R^{FF}. In some embodiments, R^{EE} is hydrogen and R^{FF} is methyl.

In some embodiments, R^{1A} is methoxy and R^{2A} and R^{3A} are both —C(═O)NH₂. In some embodiments, Y¹ is —O— and X¹ is a C3 alkylene. In some embodiments, Y¹ is —O— and X¹ is n-propylene. In some embodiments, Y¹ is —O—, X¹ is n-propylene, and Z¹ is —NH₂. In some embodiments, Y¹ is —O—, X¹ is n-propylene, and Z¹ is —NHCH₃. In some embodiments, Y¹ is —O—X¹ is n-propylene, and Z¹ is —N(CH₃)₂.

In some embodiments, R^{1A} is methoxy; R^{2A} and R^{3A} are both —C(═O)NH₂; Y¹ is —O—; X¹ is n-propylene; and Z¹ is —NH₂. In some embodiments, R^{1A} is methoxy; R^{2A} and R^{3A} are both —C(═O)NH₂; Y¹ is —O—; X¹ is n-propylene; and Z¹ is —NHCH₃. In some embodiments, R^{1A} is methoxy; R^{2A} and R^{3A} are both —C(═O)NH₂; Y¹ is —O—; X¹ is n-propylene; and Z¹ is —N(CH₃)₂.

In some embodiments, the compound of Formula (III) is

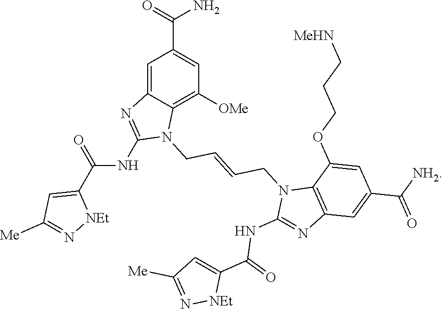

### Compounds of Formula (IV)

Some embodiments include a compound of Formula (IV):

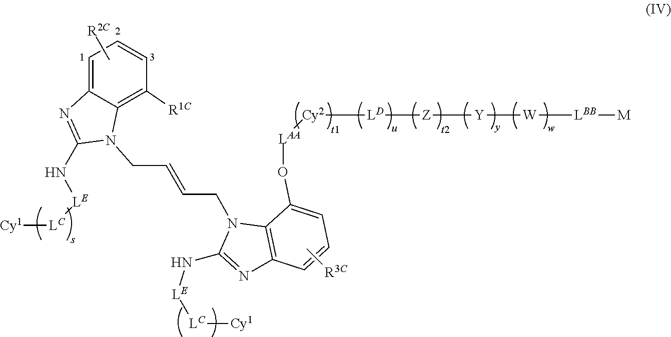

or a pharmaceutically acceptable salt thereof, wherein:
R^{1C} is hydrogen, hydroxyl, C₁₋₆ alkoxy, —(C₁₋₆ alkyl) C₁₋₆ alkoxy, —(CH₂)ₙ—NR^{A}R^{B}, or PEG2 to PEG4;
R^{2C} is —CO₂R^{M}, —(C═O)NR^{C}R^{D}, S(O)₂NR^{C}R^{D}, —S(O)₂R^{M}, —(CH₂)_{q}—NR^{E}R^{F}, —(CH₂)_{q}—OR^{M}, —O(C═O)—NR^{E}R^{F}, or —NR^{M}(C═O)—NR^{E}R^{F}, wherein R^{2C} is attached at any on of positions labeled 1, 2, or 3;
R^{3C} is —CO₂R^{M}, —(C═O)NR^{C}R^{D}, —S(O)₂NR^{C}R^{D}, —S(O)₂R^{M}, —(CH₂)_{q}—NR^{E}R^{F}, —(CH₂)_{q}—OR^{M}, —O(C═O)—NR^{E}R^{F}, or —NR^{M}(C═O)—NR^{E}R^{F}, wherein R^{3C} is attached at any one of positions labeled 1′, 2′, or 3′;
each R^{A}, R^{B}, R^{C}, R^{D}, R^{E}, R^{F}, and R^{M} are independently hydrogen or C₁₋₆ alkyl;
each subscript n is independently an integer from 0 to 6;
each subscript q is independently an integer from 0 to 6;
L^{E} is —(C═O)— or —S(O)₂—;
L^{C} is —(CR^{I}R^{J})₁₋₃—
each R^{I} and R^{J} are independently hydrogen or C₁₋₃ alkyl;
subscript s is 0 or 1;
each Cy¹ is independently a 4-6 membered heterocycle, a 5-6 membered heteroaryl, or a C₃₋₆ cycloalkyl, each optionally substituted with one or more R^{K};
each R^{K} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d2}R^{e2}, —C(O)NR^{d2}R^{e2}, —C(O)(C₁₋₆ alkyl), and —C(O)O(C₁₋₆ alkyl);
each R^{d2} and R^{e2} are independently hydrogen or C₁₋₃ alkyl;
L^{AA} is —(CH₂)₁₋₆—, —C(O)(CH₂)₁₋₆—, —C(O)NR^{L}(CH₂)₁₋₆—, —(CH₂)₁₋₆O—, —C(O)(CH₂)₁₋₆O—, or —C(O)NR^{L}(CH₂)₁₋₆O—;
R^{L} is hydrogen or C₁₋₃ alkyl;
Cy² is C34 cycloalkyl, 4-6 membered heterocycle, 5-6 membered heteroaryl, or phenyl, each optionally substituted with one or more R^{U};
each R^{U} is independently selected from the group consisting of —CO₂R^{j1}, —(C═O)NR^{d3}R^{e3}, —S(O)₂NR^{d3}R^{e3}, —(CH₂)_{q1}—NR^{g1}R^{h1}, —(CH₂)_{q1}—OR^{j1}, and —(CH₂)_{q1}—(OCH₂CH₂)₁₋₈OH;
each R^{d3}, R^{e3}, R^{g1}, R^{h1}, and R^{j1} are independently hydrogen or C₁₋₆ alkyl;
subscript q1 is an integer from 0 to 6;
subscripts t1 and t2 are independently 0 or 1, wherein at least one of t1 and t2 is 1;
L^{D} is —(CH₂)₁₋₆—;
subscript u is 0 or 1;
Z is —N(R^{HH})— or —N⁺(C₁₋₆alkyl)(R^{HH})—;
R^{HH} is hydrogen, C₁₋₆ alkyl, C₃₋₆ cycloalkyl, —(CH₂)₁₋₃C₃₋₆ cycloalkyl, —(CH₂)₁₋₃C₁₋₃ alkoxy, —(CH₂)₁₋₃ 4-6 membered heterocycle, or —(CH₂)₁₋₃ 5-6 membered heteroaryl;
Y is a self-immolative moiety, a non-self-immolative releasable moiety, or a non-cleavable moiety;
subscript y is 0 or 1;
W is a chain of 1-12 amino acids or has the structure:

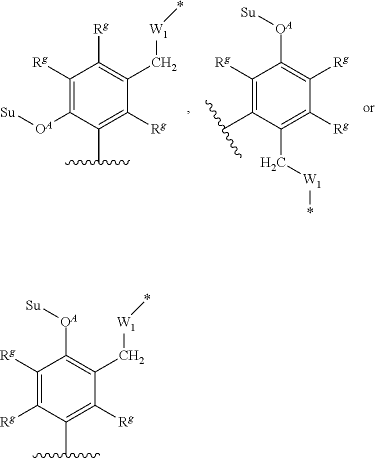

wherein Su is a Sugar moiety;
—O^{A}— represents a glycosidic bond;
each R^{g} is independently hydrogen, halogen, —CN, or —NO₂;
W¹ is absent or —O—C(═O)—;
[symbol] represents covalent attachment to L^{BB};
* represents covalent attachment to Y, L^{D}, NR^{HH}, or Cy²;
subscript w is 0 or 1;
L^{BB} is (CH₂)₁₋₆—, —C(O)CH₂)₁₋₆—, or —[NHC(OCH₂)₁₋₄]₁₋₃—; and
M is

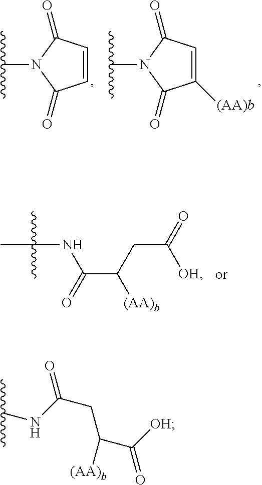

each AA is an independently selected amino acid, wherein (AA)_{b} is connected to the succinimide or hydrolyzed succinimide via a sulfur atom; and
each subscript b is independently an integer from 1 to 6.

In some embodiments, R^{1C} is hydrogen. In some embodiments, R^{1C} is hydroxyl. In some embodiments, R^{1C} is C₁₋₆ alkoxy. In some embodiments, R^{1C} is methoxy. In some embodiments, R^{1C} is —(C₁₋₆ alkyl)C₁₋₆ alkoxy. In some embodiments, R^{1C} is methoxyethyl. In some embodiments, R^{1C} is PEG2 to PEG4. In some embodiments, R^{1C} is —(CH₂)ₙ—NR^{A}R^{B}.

In some embodiments, R^{A} and R^{B} are both hydrogen. In some embodiments, R^{A} and R^{B} are independently C₁₋₃ alkyl. In some embodiments, one of R^{A} and R^{B} is hydrogen and the other of R^{A} and R^{B} is C₁₋₃ alkyl.

In some embodiments, each subscript n is 0. In some embodiments, each subscript n is 1. In some embodiments, each subscript n is 2. In some embodiments, each subscript n is 3, 4, 5, or 6.

In some embodiments, R^{2C} and R^{3C} are independently —CO₂H, —(C═O)ₘ—NR^{C}R^{D}, or —(CH₂)_{q}—NR^{E}R^{F}; and R^{2C} and R^{3C} are the same. In some embodiments, R^{2C} and R^{3C} are independently —CO₂H, —(C═O)ₘ—NR^{C}R^{D}, or —(CH₂)_{q}—NR^{E}R^{F}; and R^{2C} and R^{3C} are different. In some embodiments, R^{2C} is —(C═O)ₘ—NR^{C}R^{D}. In some embodiments, R^{3C} is —(C═O)ₘ—NR^{C}R^{D}. In some embodiments, R^{C} and R^{D} are both hydrogen. In some embodiments, R^{C} and R^{D} are each independently C₁₋₃ alkyl. In some embodiments, one of R^{C} and R^{D} is hydrogen and the other of R^{C} and R^{D} is C₁₋₃ alkyl. In some embodiments, each subscript m is 0. In some embodiments, each subscript m is 1.

In some embodiments, R^{2C} is —(CH₂)_{q}—NR^{E}R^{F}. In some embodiments, R^{3C} is —(CH₂)_{q}—NR^{E}R^{F}. In some embodiments, R^{E} and R^{F} are both hydrogen. In some embodiments, R^{E} and R^{F} are each independently C₁₋₃ alkyl. In some embodiments, one of R^{E} and R^{F} is hydrogen and the other of R^{E} and R^{F} is C₁₋₃ alkyl. In some embodiments, each subscript q is 0. In some embodiments, each subscript q is an integer from 1 to 6.

In some embodiments, R^{2C} is —CO₂R^{M}. In some embodiments, R^{3C} is —CO₂R^{M}. In some embodiments, R^{M} is hydrogen. In some embodiments, R^{M} is C₁₋₃ alkyl.

In some embodiments, R^{2C} is —(CH₂)_{q}—OR^{M}.

In some embodiments, R^{3C} is —(CH₂)_{q}—OR^{M}. In some embodiments, R^{M} is hydrogen. In some embodiments, q is 0. In some embodiments, q is 1.

In some embodiments, R^{2C} is —O(C═O)—NR^{E}R^{F}. In some embodiments, R^{3C} is —O(C═O)—NR^{E}R^{F}. In some embodiments, R^{E} and R^{F} are both hydrogen. In some embodiments, R^{E} and R^{F} are each independently C₁₋₃ alkyl. In some embodiments, R^{E} and R^{F} is hydrogen and the other of R^{E} and R^{F} is C₁₋₃ alkyl.

In some embodiments, R^{2C} is —NR^{M}(C═O)—NR^{E}R^{F}. In some embodiments, R^{3C} is —NR^{M}(C═O)—NR^{E}R^{F}. In some embodiments, R^{E}, R^{F}, and R^{M} are all hydrogen. In some embodiments, R^{E}, R^{F}, and R^{M} are each independently C₁₋₃ alkyl. In some embodiments, one of R^{E}, R^{F}, and R^{M} is C₁₋₃ alkyl and the rest of R^{E}, R^{F}, and R^{M} is hydrogen.

In some embodiments, R^{2C} is —S(O)₂NR^{C}R^{D}. In some embodiments, R^{3C} is —S(O)₂NR^{C}R^{D}. In some embodiments, R^{C} and R^{D} are both hydrogen. In some embodiments, R^{C} and R^{D} are each independently C₁₋₃ alkyl. In some embodiments, one of R^{C} and R^{D} is hydrogen and the other of R^{C} and R^{D} is C₁₋₃ alkyl.

In some embodiments, R^{2C} is —S(O)₂R^{M}. In some embodiments, R^{3C} is —S(O)₂R^{M}. In some embodiments, R^{M} is hydrogen. In some embodiments, R^{M} is C₁₋₃ alkyl.

In some embodiments, R^{2C} is attached at position 1. In some embodiments, R^{2C} is attached at position 2. In some embodiments, R^{2C} is attached at position 3. In some embodiments, R^{3C} is attached at position 1′. In some embodiments, R^{3C} is attached at position 2′. In some embodiments, R^{3C} is attached at position 3′.

In some embodiments, L^{E} is —(C═O)—. In some embodiments, L^{E} is —S(O)₂—.

In some embodiments, each R^{I} and R^{J} is hydrogen. In some embodiments, each R^{I} and R^{J} is C₁₋₃ alkyl. In some embodiments, one of R^{I} and R^{J} is hydrogen and the other of R^{I} and R^{J} is C₁₋₃ alkyl.

In some embodiments, L^{C} is —(CR^{I}R^{J})—.

In some embodiments, s is 0. In some embodiments, s is 1.

In some embodiments, each Cy¹ is independently a 5-6 membered heteroaryl. In some embodiments, each Cy¹ is pyrazole optionally substituted with one or more R^{K}. In some embodiments, each Cy¹ is independently selected from the group consisting of pyrazole, imidazole, furan, thiophene, thiazole, isothiazole, oxazole, isoxazole, pyrrole, pyridazine, pyridine, pyrimidine, and pyrazine, each optionally substituted with one or more R^{K}. In some embodiments, each Cy¹ is independently selected from the group consisting of imidazole, furan, thiophene, thiazole, isothiazole, oxazole, isoxazole, pyrrole, pyridazine, pyridine, pyrimidine, and pyrazine, each optionally substituted with one or more R^{K}. In some embodiments, each Cy¹ is independently a C4-s cycloalkyl optionally substituted with one or more R^{K}. In some embodiments, each R^{K} is independently selected from the group consisting of C₁₋₃ alkyl, C₁₋₃ haloalkyl, and halogen. In some embodiments, each R^{K} is independently selected from the group consisting of methyl, ethyl, —CF₃, and halogen.

In some embodiments, each Cy¹ is the same. In some embodiments, each Cy¹ is different.

In some embodiments, L^{AA} is —(CH₂)₁₋₆—. In some embodiments, L^{AA} is —(CH₂)₁₋₃—. In some embodiments, L^{AA} is —(CH₂)₁₋₆O—. In some embodiments, L^{AA} is —(CH₂)₁₋₃O—.

In some embodiments, Cy² is a 4-6 membered heterocycle. In some embodiments, Cy² has the structure:

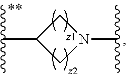

wherein each of subscripts z1 and z2 is independently an integer from 1 to 3 and ** indicates attachment to L^{AA}. In some embodiments, z1 and z2 are 1. In some embodiments, z1 and z2 are 2. In some embodiments, z1 is 1 and z2 is 2.

In some embodiments, Cy² has the structure:

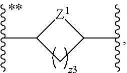

wherein
Z¹ is selected from the group consisting of —O—, —S—, —CR^{N}R^{O}—, and —NR^{P}—;
R^{N}, R^{O}, and R^{P} are independently hydrogen or C₁₋₆ alkyl;
subscript z3 is an integer from 1 to 3; and
** indicates attachment to L^{AA}.

In some embodiments, R^{N} and R^{O} are hydrogen. In some embodiments, R^{P} is hydrogen. In some embodiments, R^{P} is methyl.

In some embodiments, Cy² is a 5-6 membered heteroaryl. In some embodiments, Cy² is selected from the group consisting of:

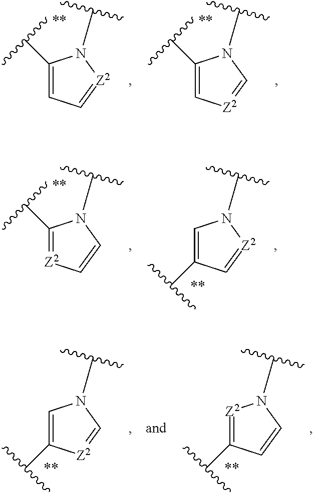

wherein
Z² is ═CR^{N}— or ═N;
R^{N} is hydrogen or C₁₋₄ alkyl; and
** indicates attachment to L^{AA}

In some embodiments, Z² is ═CR^{N} and R^{N} is hydrogen. In some embodiments, Z² is ═N—.

In some embodiments, Cy² is selected from the group consisting of:

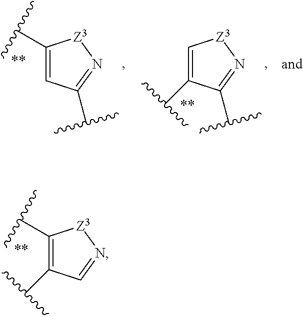

wherein Z³ is —O— or —S— and ** indicates attachment to L^{AA}, L^{D}, NR^{HH}, Y, W, or L^{BB}.

In some embodiments, ** indicates attachment to L^{AA}. In some embodiments, ** indicates attachment to L^{D}, NR^{HH}, Y, W, or L^{B}.

In some embodiments, Cy² is selected from the group consisting of:

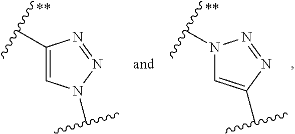 [SMILES: *c1cn(*)nn1 ; *c1cn(*)nn1]

wherein ** indicates attachment to L^{AA}.

In some embodiments, Cy² is selected from the group consisting of:

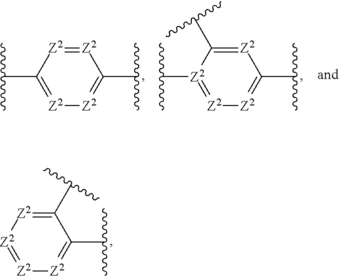

wherein
each Z² is independently ═CR^{N}— or ═N—; and
each R^{N} is hydrogen or C₁₋₆ alkyl.

In some embodiments, at least one Z² is ═N—. In some embodiments, one Z² is ═N— and the remaining Z² are ═CR^{N}—. In some embodiments, two Z² are ═N— and the remaining Z2 are ═CR^{N}—.

In some embodiments, R^{N} is hydrogen.

In some embodiments, Cy² is selected from the group consisting of:

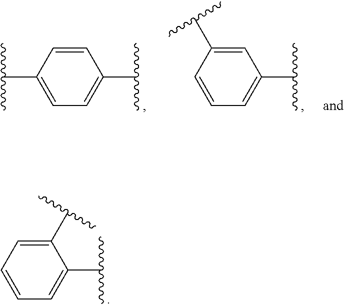 [SMILES: *c1ccc(*)cc1 ; *c1cccc(*)c1 ; *c1ccccc1*]

In some embodiments, Cy² is cyclobutyl.

In some embodiments, each R^{d3}, R^{e3}, R^{g1}, R^{h1}, and R^{j1} are independently hydrogen or —CH₃.

In some embodiments, each R^{U} is independently selected from —CO₂H, —(C═O)NH₂, —S(O)₂NH₂, —CH₂NH₂, and —CH₂OH.

In some embodiments, t1 is 0 and t2 is 1. In some embodiments, t1 is 1 and t2 is 0. In some embodiments, t1 is 1 and t2 is 1.

In some embodiments, u is 1 and L^{D} is —(CH₂)₁₋₃. In some embodiments, u is 0.

In some embodiments, t2 is 1 and R^{HH} is hydrogen. In some embodiments, t2 is 1 and R^{HH} is C₁₋₃ alkyl. In some embodiments, t2 is 1 and R^{HH} is C₃₋₄ cycloalkyl. In some embodiments, t2 is 1 and R^{HH} is —(CH₂) C₃₋₄ cycloalkyl. In some embodiments, t2 is 1 and R^{HH} is —(CH₂) 4-5 membered heterocycle. In some embodiments, t2 is 1 and R^{HH} is —(CH₂) 5-membered heteroaryl.

In some embodiments, Z is —N(R^{HH})—. In other embodiments, Z is —N⁺(C₁₋₆ alkyl)(R^{HH})—.

In some embodiments, Y is

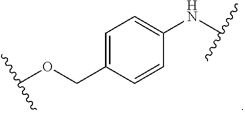 [SMILES: *Nc1ccc(CO*)cc1]

In some embodiments, Y is a cyclohexanecarboxyl, undecanoyl, caproyl, hexanoyl, butanoyl or propionyl group. In some embodiments, Y is PEG4 to PEG12. In some embodiments, y is 0. In some embodiments, y is 1.

In some embodiments, W is a chain of 1-12 amino acids. In some embodiments, W is a chain of 1-6 amino acids. In some embodiments, W is a chain of 1-3 amino acids.

In some embodiments, W is independently selected from the group consisting of alanine, valine, isoleucine, leucine, aspartic acid, glutamic acid, lysine, histidine, arginine, glycine, serine, threonine, phenylalanine, O-methylserine, O-methylaspartic acid, O-methylglutamic acid, N-methyllysine, O-methyltyrosine, O-methylhistidine, and O-methylthreonine. In some embodiments, each amino acid in W is independently selected from the group consisting of alanine, glycine, lysine, serine, aspartic acid, aspartate methyl ester, N,N-dimethyl-lysine, phenylalanine, citrulline, valine-alanine, valine-citrulline, phenylalanine-lysine or homoserine methyl ether.

In some embodiments, W has the structure:

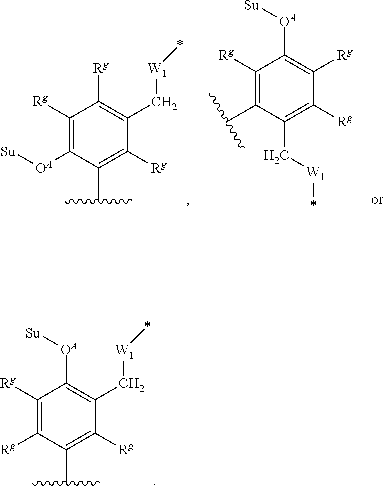

In some embodiments, W¹ is —O—C(═O)—. In some embodiments, one R^{g} is halogen, —CN, or —NO₂, and the remaining R^{G} are hydrogen. In some embodiments, each R^{g} is hydrogen.

In some embodiments, w is 0. In some embodiments, w is 1.

In some embodiments, L^{BB} is —(CH₂)₁₋₃—. In some embodiments, L^{BB} is —C(O)(CH₂)₁₋₂—.

In some embodiments, L^{BB} is —C(O)(CH₂)₂—. In some embodiments, L^{BB} is —[NHC(O)(CH₂)₂]₁₋₂—. In some embodiments, L^{BB} is —[NHC(O)(CH₂)₂]₂—.

In some embodiments, M is

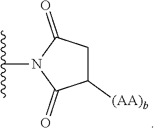

In some aspects, M is

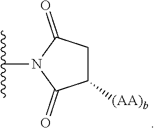

In some aspects, M is

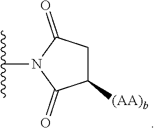

In some embodiments, M is

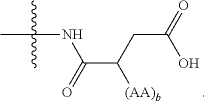

In some aspects, M is

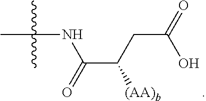

In some aspects, M is

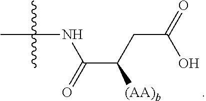

In some aspects, M is

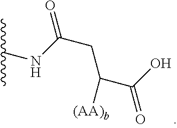

n some aspects, M is

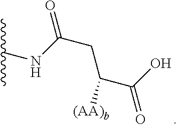

In some aspects, M is

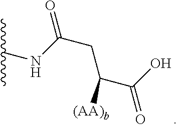

In some embodiments, each AA is independently a natural amino acid; wherein (AA)b is connected to the succinimide or hydrolyzed succinimide via a sulfur atom. In some embodiments, each AA is independently a natural amino acid; wherein (AA)_{b} is connected to the succinimide or hydrolyzed succinimide via a nitrogen atom. In some embodiments, each subscript b is 1. In some embodiments, each subscript b is 2. In some embodiments, each subscript b is 3, 4, 5, or 6.

In some embodiments, M is

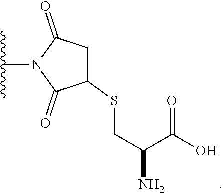 [SMILES: *N1C(=O)CC(SC[C@H](N)C(=O)O)C1=O]

In some aspects, M is

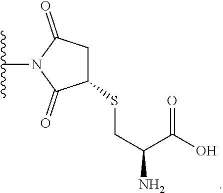 [SMILES: *N1C(=O)C[C@H](SC[C@H](N)C(=O)O)C1=O]

In some aspects, M is

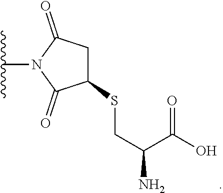 [SMILES: *N1C(=O)C[C@@H](SC[C@H](N)C(=O)O)C1=O]

In some embodiments, M is

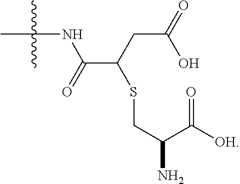 [SMILES: *NC(=O)C(CC(=O)O)SC[C@H](N)C(=O)O]

n some aspects, M is

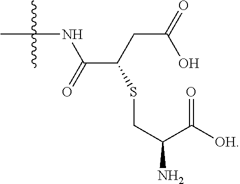 [SMILES: *NC(=O)[C@H](CC(=O)O)SC[C@H](N)C(=O)O]

In some aspects, M is

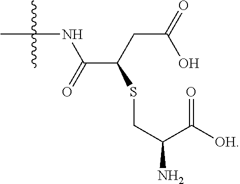 [SMILES: *NC(=O)[C@@H](CC(=O)O)SC[C@H](N)C(=O)O]

In some embodiments, M is

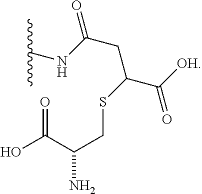 [SMILES: *NC(=O)CC(SC[C@H](N)C(=O)O)C(=O)O]

In some aspects, M is

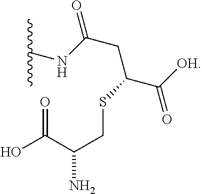 [SMILES: *NC(=O)C[C@@H](SC[C@H](N)C(=O)O)C(=O)O]

In some aspects, M is

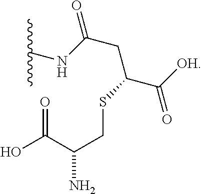 [SMILES: *NC(=O)C[C@@H](SC[C@H](N)C(=O)O)C(=O)O]

In some embodiments, M is

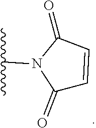 [SMILES: *N1C(=O)C=CC1=O]

Some embodiments of the compound of Formula (IV) include a compound selected from the group consisting of:

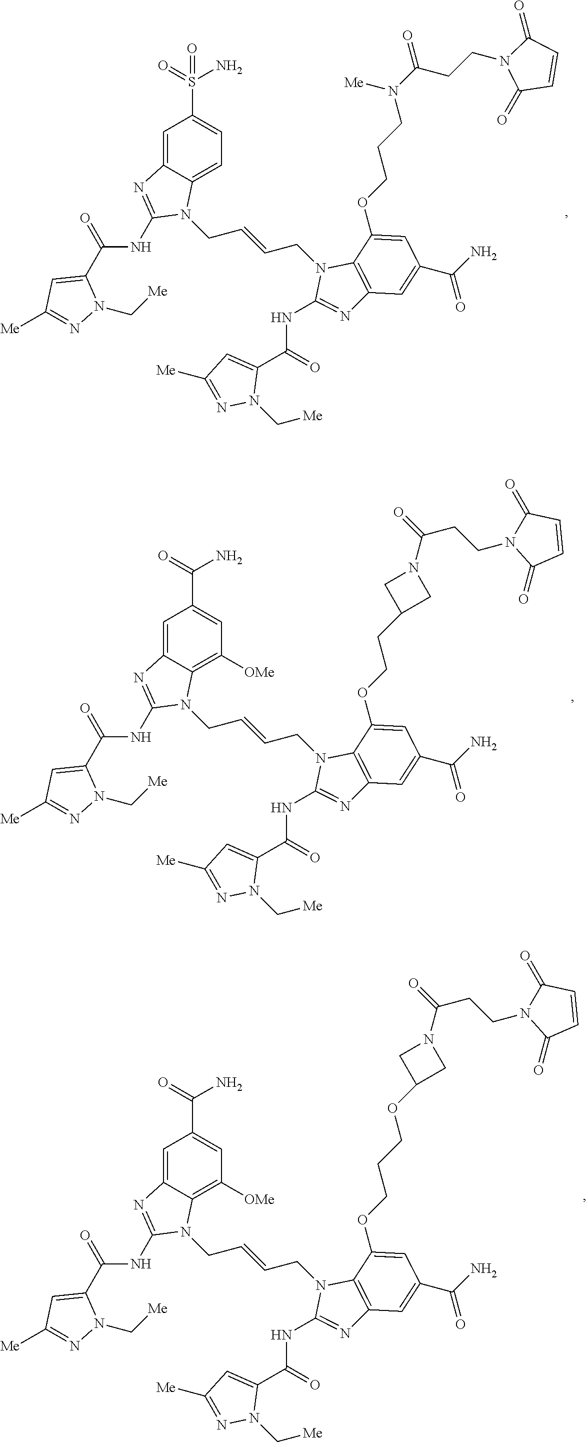 [SMILES: CCn1nc(C)cc1C(=O)Nc1nc2cc(S(N)(=O)=O)ccc2n1C/C=C/Cn1c(NC(=O)c2cc(C)nn2CC)nc2cc(C(N)=O)cc(OCCCN(C)C(=O)CCN3C(=O)C=CC3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCC3CN(C(=O)CCN4C(=O)C=CC4=O)C3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCOC3CN(C(=O)CCN4C(=O)C=CC4=O)C3)c21]

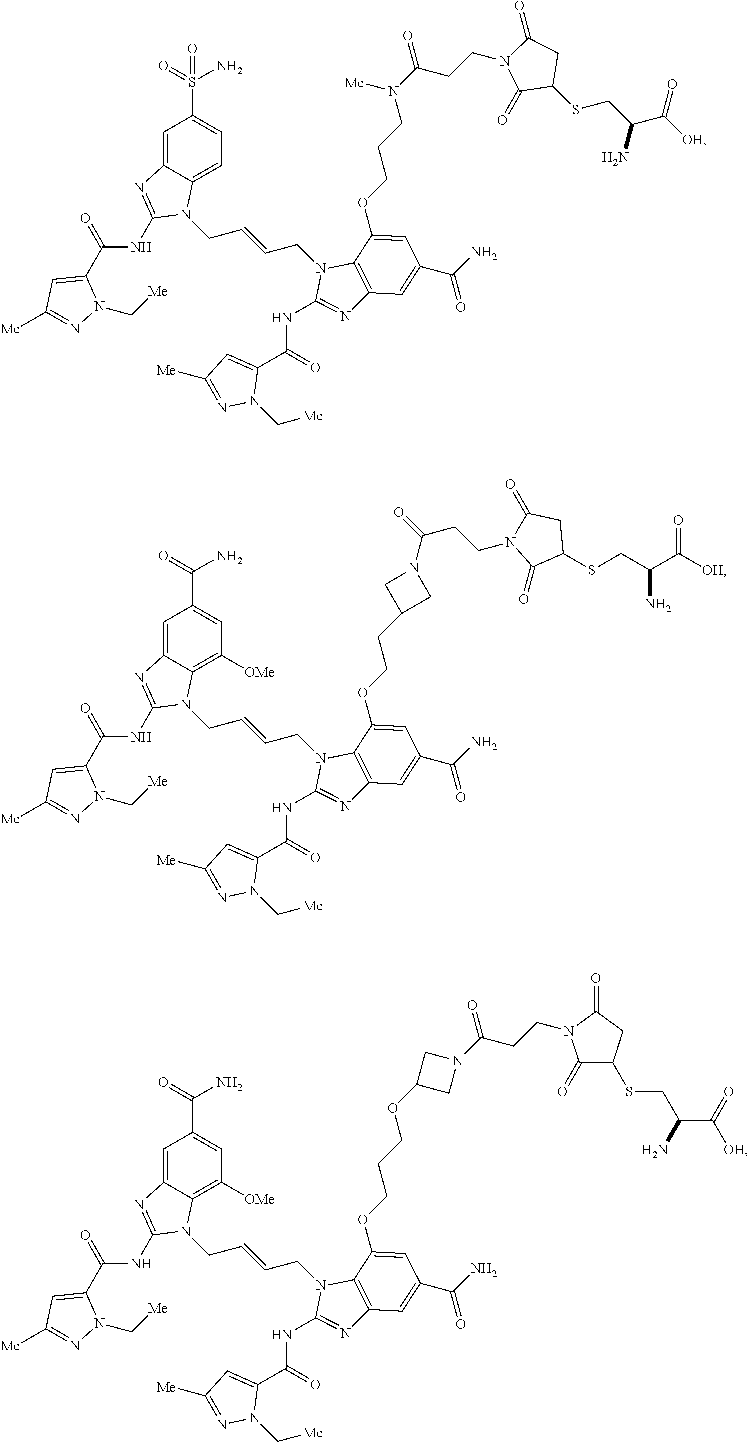 [SMILES: CCn1nc(C)cc1C(=O)Nc1nc2cc(S(N)(=O)=O)ccc2n1C/C=C/Cn1c(NC(=O)c2cc(C)nn2CC)nc2cc(C(N)=O)cc(OCCCN(C)C(=O)CCN3C(=O)CC(SC[C@H](N)C(=O)O)C3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCC3CN(C(=O)CCN4C(=O)CC(SC[C@H](N)C(=O)O)C4=O)C3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCOC3CN(C(=O)CCN4C(=O)CC(SC[C@H](N)C(=O)O)C4=O)C3)c21]

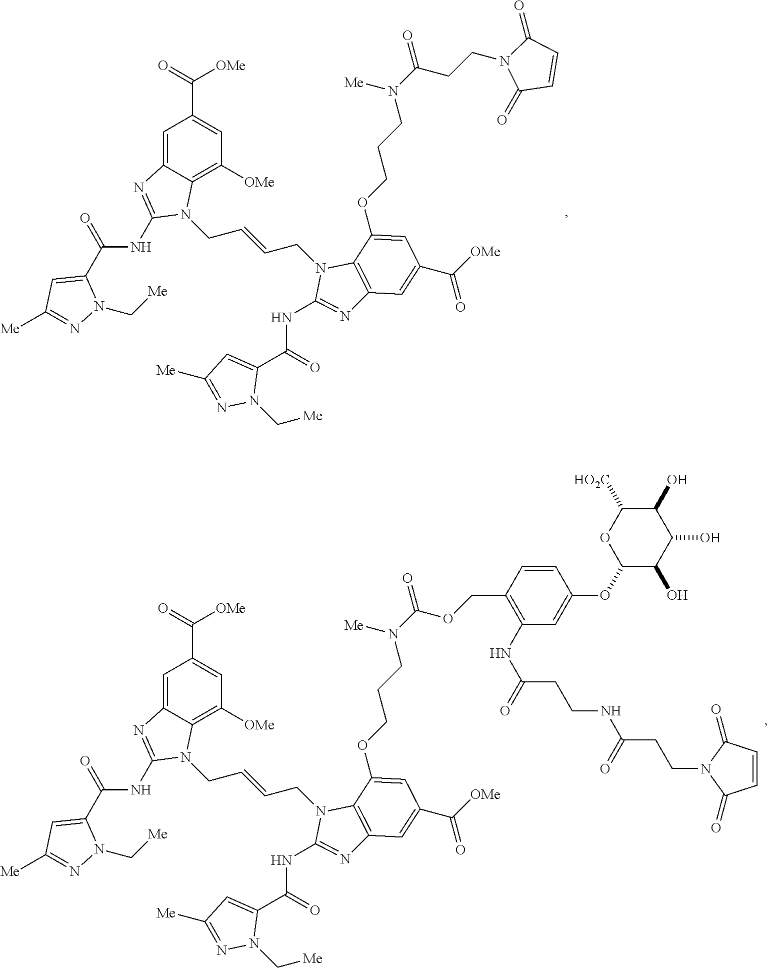

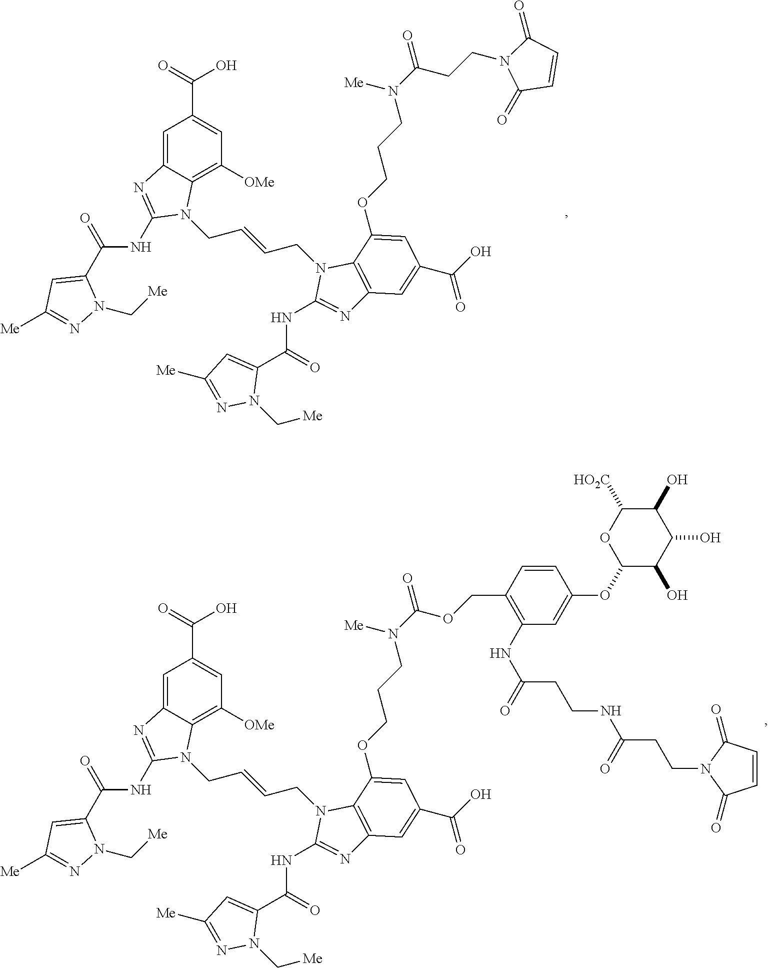

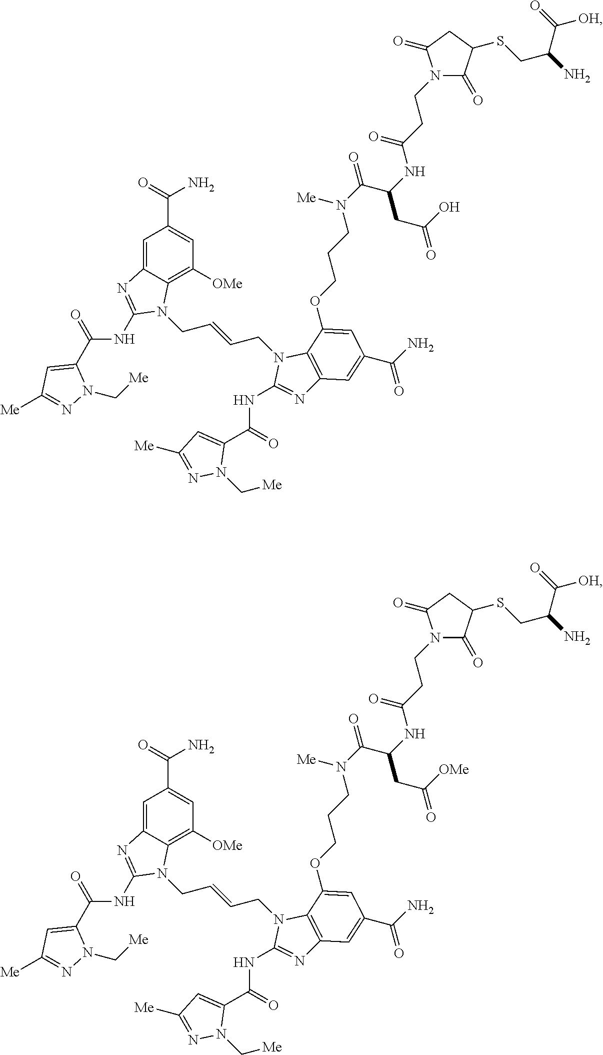 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CC(=O)O)NC(=O)CCN3C(=O)CC(SC[C@H](N)C(=O)O)C3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CC(=O)OC)NC(=O)CCN3C(=O)CC(SC[C@H](N)C(=O)O)C3=O)c21]

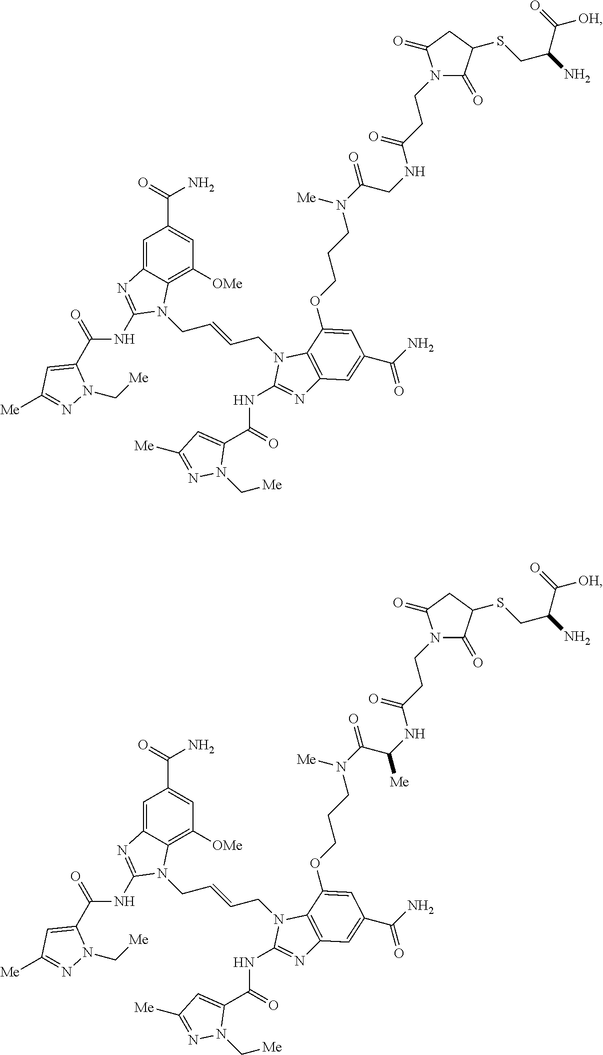 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)CNC(=O)CCN3C(=O)CC(SC[C@H](N)C(=O)O)C3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](C)NC(=O)CCN3C(=O)CC(SC[C@H](N)C(=O)O)C3=O)c21]

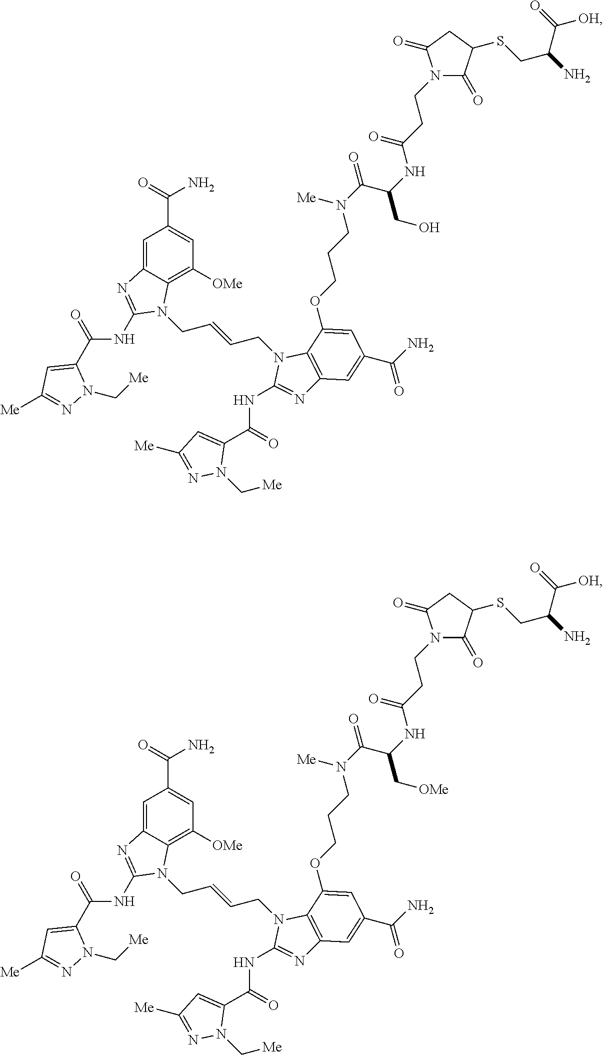 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CO)NC(=O)CCN3C(=O)CC(SC[C@H](N)C(=O)O)C3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](COC)NC(=O)CCN3C(=O)CC(SC[C@H](N)C(=O)O)C3=O)c21]

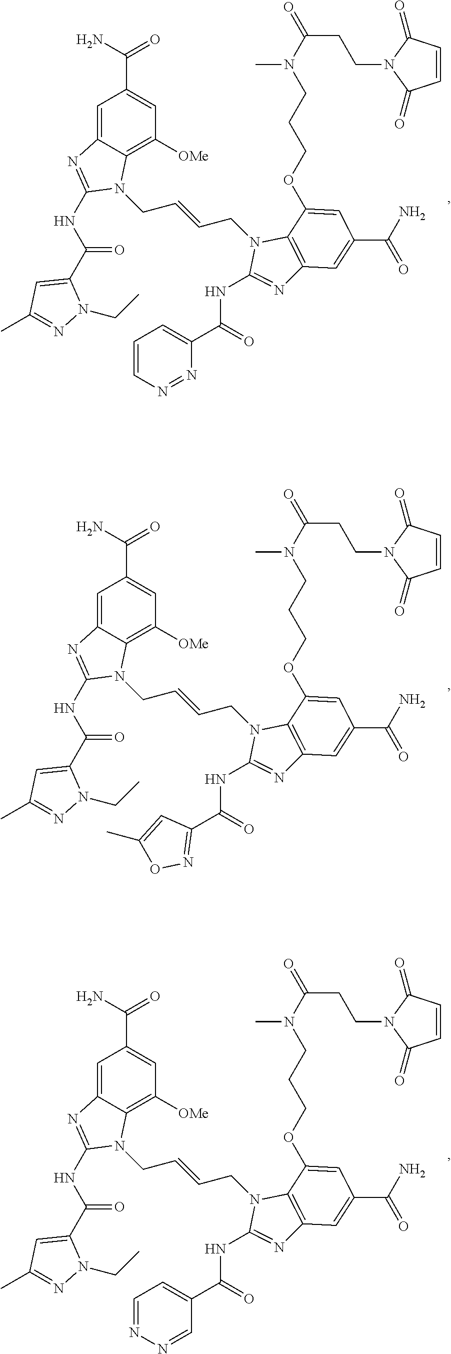 [SMILES: CCn1nc(C)cc1C(=O)Nc1nc2cc(C(N)=O)cc(OC)c2n1C/C=C/Cn1c(NC(=O)c2cccnn2)nc2cc(C(N)=O)cc(OCCCN(C)C(=O)CCN3C(=O)C=CC3=O)c21 ; CCn1nc(C)cc1C(=O)Nc1nc2cc(C(N)=O)cc(OC)c2n1C/C=C/Cn1c(NC(=O)c2cc(C)on2)nc2cc(C(N)=O)cc(OCCCN(C)C(=O)CCN3C(=O)C=CC3=O)c21 ; CCn1nc(C)cc1C(=O)Nc1nc2cc(C(N)=O)cc(OC)c2n1C/C=C/Cn1c(NC(=O)c2ccnnc2)nc2cc(C(N)=O)cc(OCCCN(C)C(=O)CCN3C(=O)C=CC3=O)c21]

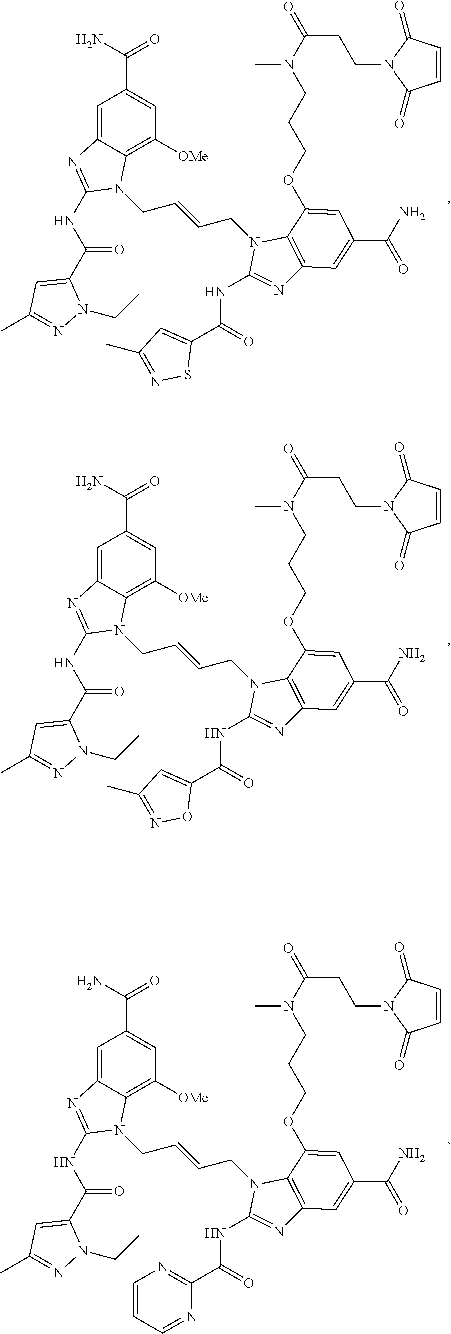 [SMILES: CCn1nc(C)cc1C(=O)Nc1nc2cc(C(N)=O)cc(OC)c2n1C/C=C/Cn1c(NC(=O)c2cc(C)ns2)nc2cc(C(N)=O)cc(OCCCN(C)C(=O)CCN3C(=O)C=CC3=O)c21 ; CCn1nc(C)cc1C(=O)Nc1nc2cc(C(N)=O)cc(OC)c2n1C/C=C/Cn1c(NC(=O)c2cc(C)no2)nc2cc(C(N)=O)cc(OCCCN(C)C(=O)CCN3C(=O)C=CC3=O)c21 ; CCn1nc(C)cc1C(=O)Nc1nc2cc(C(N)=O)cc(OC)c2n1C/C=C/Cn1c(NC(=O)c2ncccn2)nc2cc(C(N)=O)cc(OCCCN(C)C(=O)CCN3C(=O)C=CC3=O)c21]

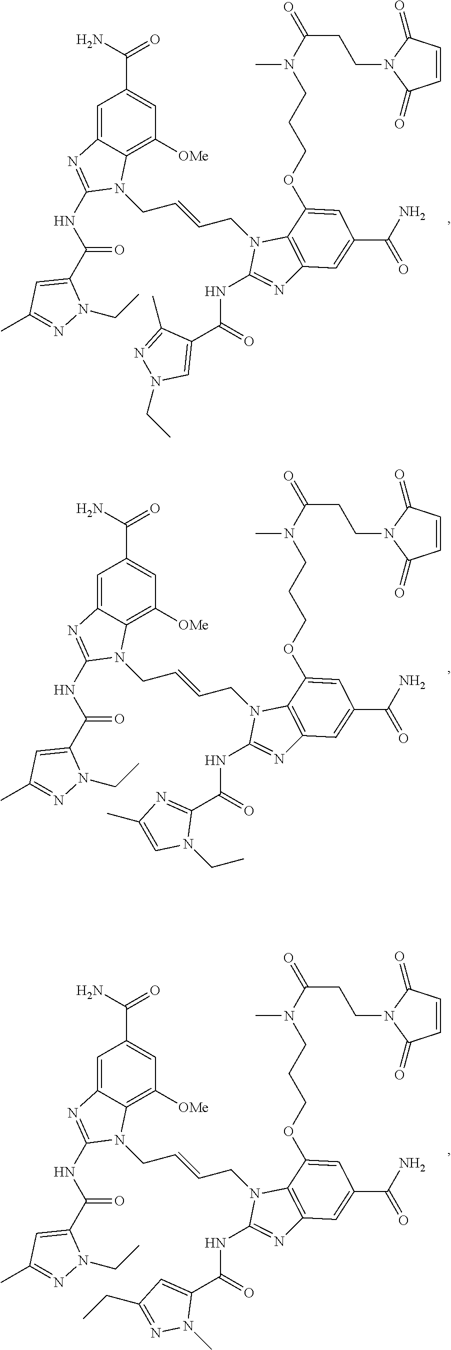 [SMILES: CCn1cc(C(=O)Nc2nc3cc(C(N)=O)cc(OCCCN(C)C(=O)CCN4C(=O)C=CC4=O)c3n2C/C=C/Cn2c(NC(=O)c3cc(C)nn3CC)nc3cc(C(N)=O)cc(OC)c32)c(C)n1 ; CCn1cc(C)nc1C(=O)Nc1nc2cc(C(N)=O)cc(OCCCN(C)C(=O)CCN3C(=O)C=CC3=O)c2n1C/C=C/Cn1c(NC(=O)c2cc(C)nn2CC)nc2cc(C(N)=O)cc(OC)c21 ; CCc1cc(C(=O)Nc2nc3cc(C(N)=O)cc(OCCCN(C)C(=O)CCN4C(=O)C=CC4=O)c3n2C/C=C/Cn2c(NC(=O)c3cc(C)nn3CC)nc3cc(C(N)=O)cc(OC)c32)n(C)n1]

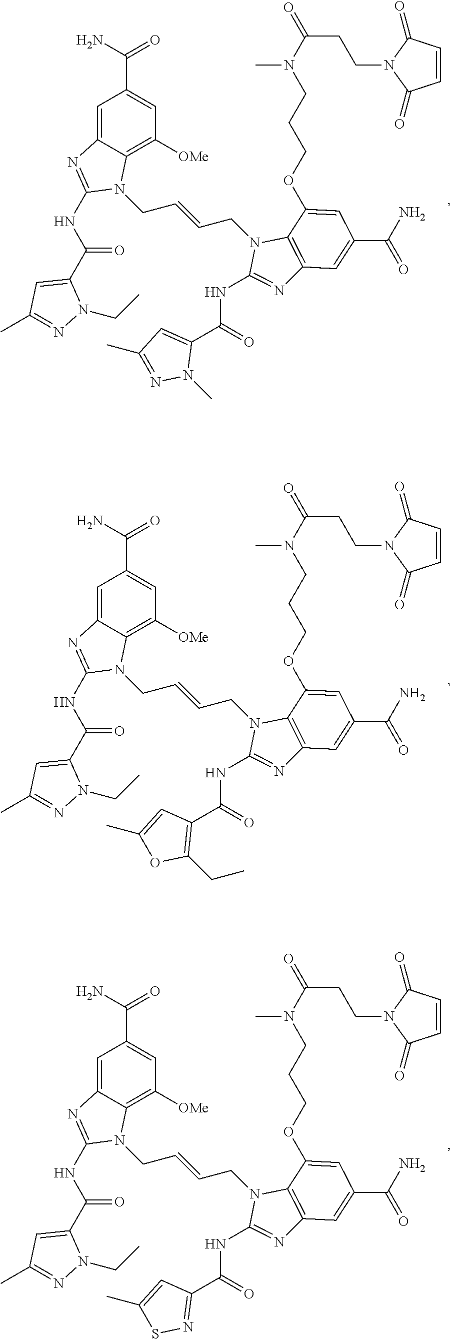 [SMILES: CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCCN(C)C(=O)CCN3C(=O)C=CC3=O)c21 ; CCc1oc(C)cc1C(=O)Nc1nc2cc(C(N)=O)cc(OCCCN(C)C(=O)CCN3C(=O)C=CC3=O)c2n1C/C=C/Cn1c(NC(=O)c2cc(C)nn2CC)nc2cc(C(N)=O)cc(OC)c21 ; CCn1nc(C)cc1C(=O)Nc1nc2cc(C(N)=O)cc(OC)c2n1C/C=C/Cn1c(NC(=O)c2cc(C)sn2)nc2cc(C(N)=O)cc(OCCCN(C)C(=O)CCN3C(=O)C=CC3=O)c21]

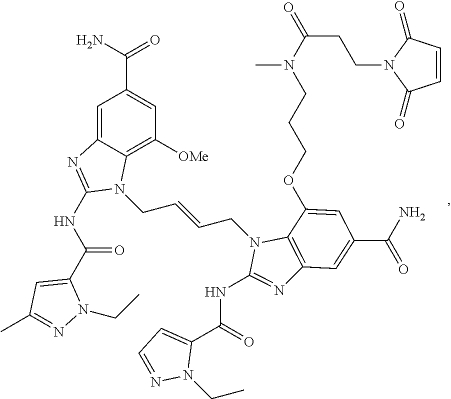 [SMILES: CCn1nc(C)cc1C(=O)Nc1nc2cc(C(N)=O)cc(OC)c2n1C/C=C/Cn1c(NC(=O)c2ccnn2CC)nc2cc(C(N)=O)cc(OCCCN(C)C(=O)CCN3C(=O)C=CC3=O)c21]

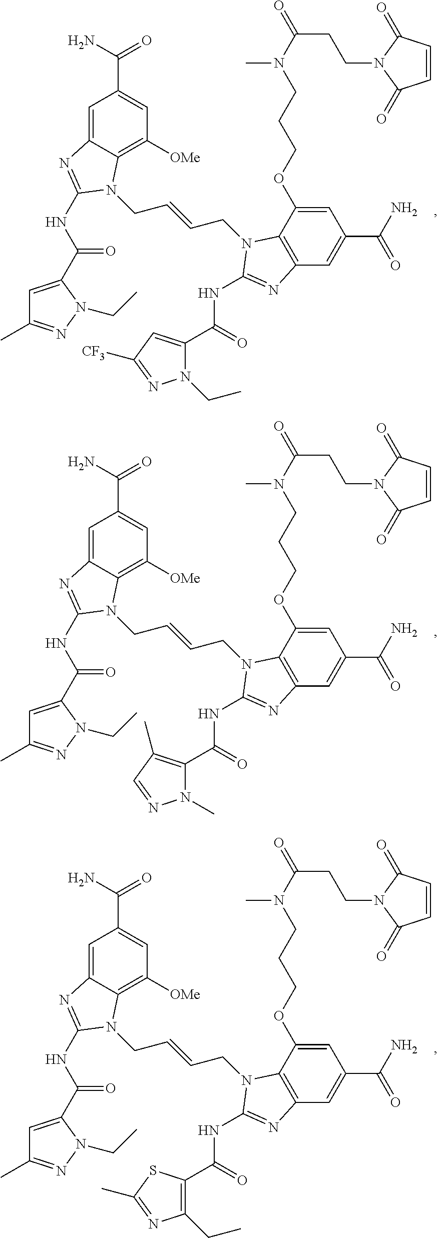 [SMILES: CCn1nc(C)cc1C(=O)Nc1nc2cc(C(N)=O)cc(OC)c2n1C/C=C/Cn1c(NC(=O)c2cc(C(F)(F)F)nn2CC)nc2cc(C(N)=O)cc(OCCCN(C)C(=O)CCN3C(=O)C=CC3=O)c21 ; CCn1nc(C)cc1C(=O)Nc1nc2cc(C(N)=O)cc(OC)c2n1C/C=C/Cn1c(NC(=O)c2c(C)cnn2C)nc2cc(C(N)=O)cc(OCCCN(C)C(=O)CCN3C(=O)C=CC3=O)c21 ; CCc1nc(C)sc1C(=O)Nc1nc2cc(C(N)=O)cc(OCCCN(C)C(=O)CCN3C(=O)C=CC3=O)c2n1C/C=C/Cn1c(NC(=O)c2cc(C)nn2CC)nc2cc(C(N)=O)cc(OC)c21]

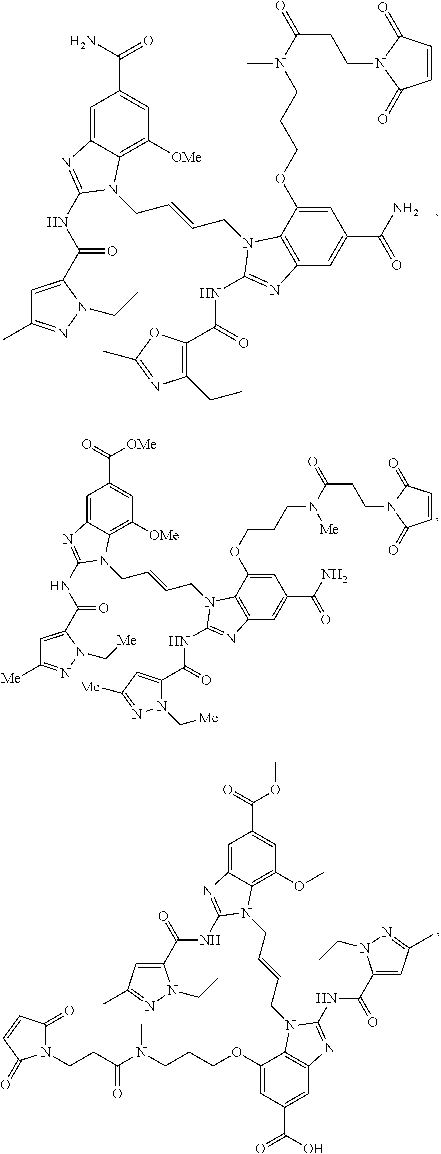 [SMILES: CCc1nc(C)oc1C(=O)Nc1nc2cc(C(N)=O)cc(OCCCN(C)C(=O)CCN3C(=O)C=CC3=O)c2n1C/C=C/Cn1c(NC(=O)c2cc(C)nn2CC)nc2cc(C(N)=O)cc(OC)c21 ; CCn1nc(C)cc1C(=O)Nc1nc2cc(C(=O)OC)cc(OC)c2n1C/C=C/Cn1c(NC(=O)c2cc(C)nn2CC)nc2cc(C(N)=O)cc(OCCCN(C)C(=O)CCN3C(=O)C=CC3=O)c21 ; CCn1nc(C)cc1C(=O)Nc1nc2cc(C(=O)OC)cc(OC)c2n1C/C=C/Cn1c(NC(=O)c2cc(C)nn2CC)nc2cc(C(=O)O)cc(OCCCN(C)C(=O)CCN3C(=O)C=CC3=O)c21]

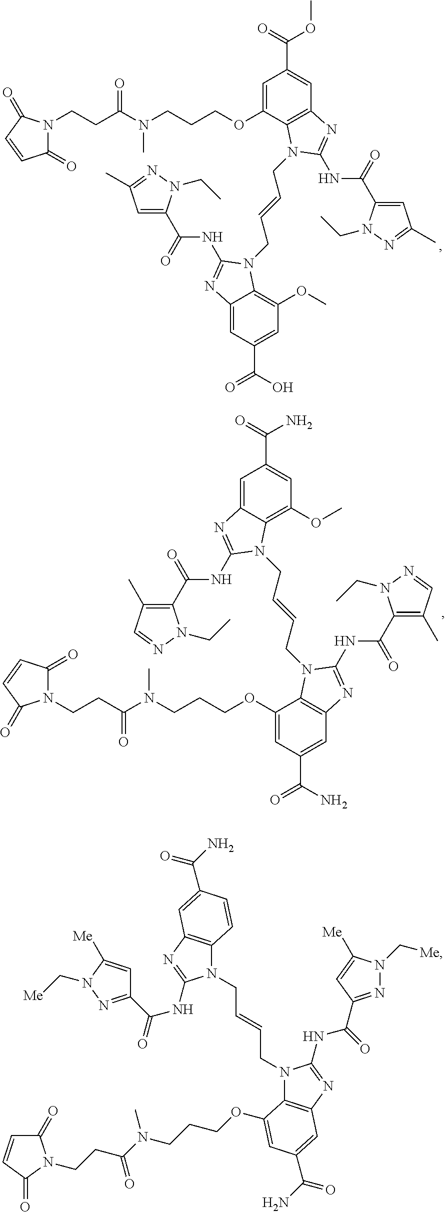

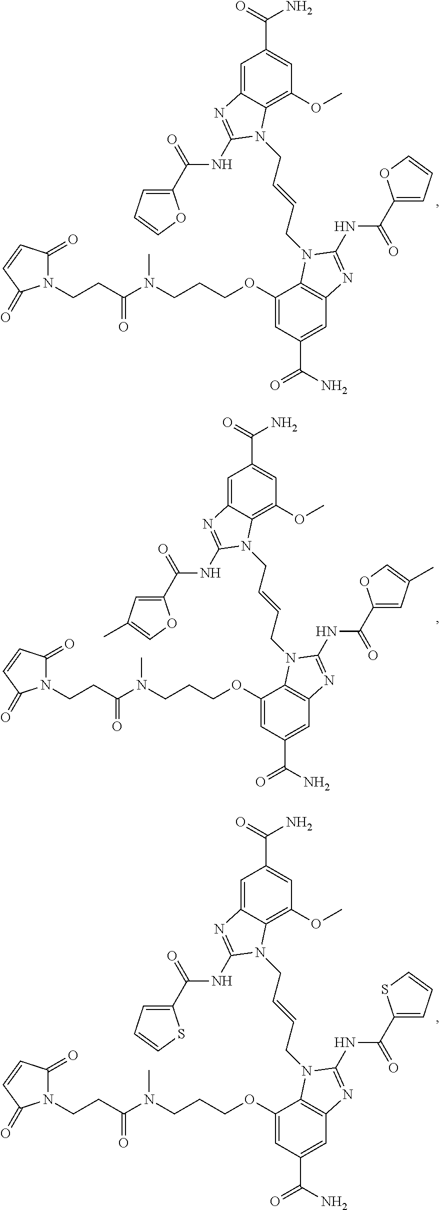

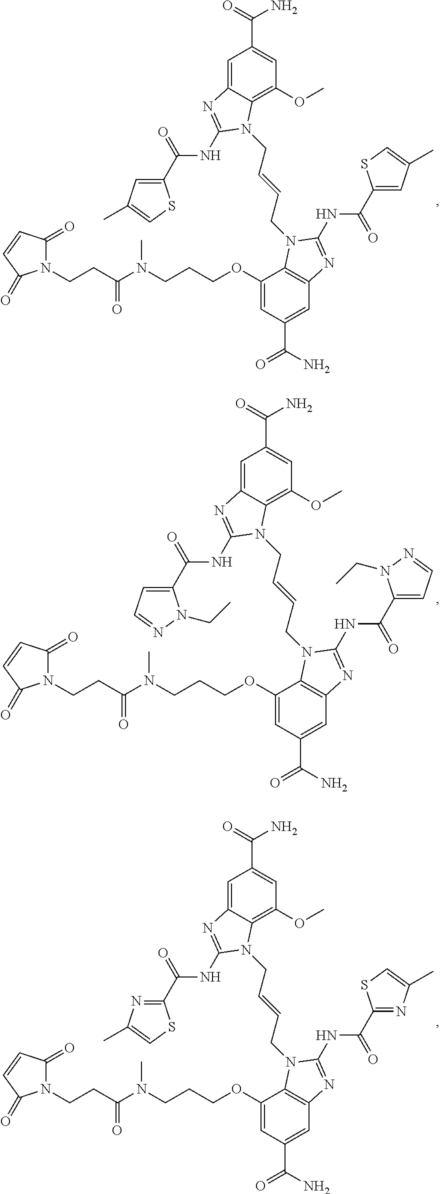 [SMILES: COc1cc(C(N)=O)cc2nc(NC(=O)c3cc(C)cs3)n(C/C=C/Cn3c(NC(=O)c4cc(C)cs4)nc4cc(C(N)=O)cc(OCCCN(C)C(=O)CCN5C(=O)C=CC5=O)c43)c12 ; CCn1nccc1C(=O)Nc1nc2cc(C(N)=O)cc(OC)c2n1C/C=C/Cn1c(NC(=O)c2ccnn2CC)nc2cc(C(N)=O)cc(OCCCN(C)C(=O)CCN3C(=O)C=CC3=O)c21 ; COc1cc(C(N)=O)cc2nc(NC(=O)c3nc(C)cs3)n(C/C=C/Cn3c(NC(=O)c4nc(C)cs4)nc4cc(C(N)=O)cc(OCCCN(C)C(=O)CCN5C(=O)C=CC5=O)c43)c12]

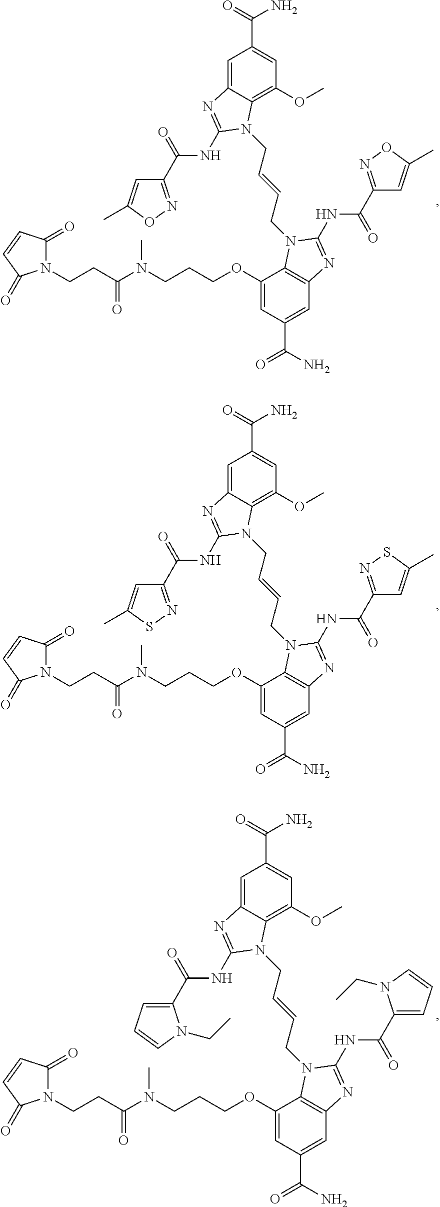 [SMILES: COc1cc(C(N)=O)cc2nc(NC(=O)c3cc(C)on3)n(C/C=C/Cn3c(NC(=O)c4cc(C)on4)nc4cc(C(N)=O)cc(OCCCN(C)C(=O)CCN5C(=O)C=CC5=O)c43)c12 ; COc1cc(C(N)=O)cc2nc(NC(=O)c3cc(C)sn3)n(C/C=C/Cn3c(NC(=O)c4cc(C)sn4)nc4cc(C(N)=O)cc(OCCCN(C)C(=O)CCN5C(=O)C=CC5=O)c43)c12 ; CCn1cccc1C(=O)Nc1nc2cc(C(N)=O)cc(OC)c2n1C/C=C/Cn1c(NC(=O)c2cccn2CC)nc2cc(C(N)=O)cc(OCCCN(C)C(=O)CCN3C(=O)C=CC3=O)c21]

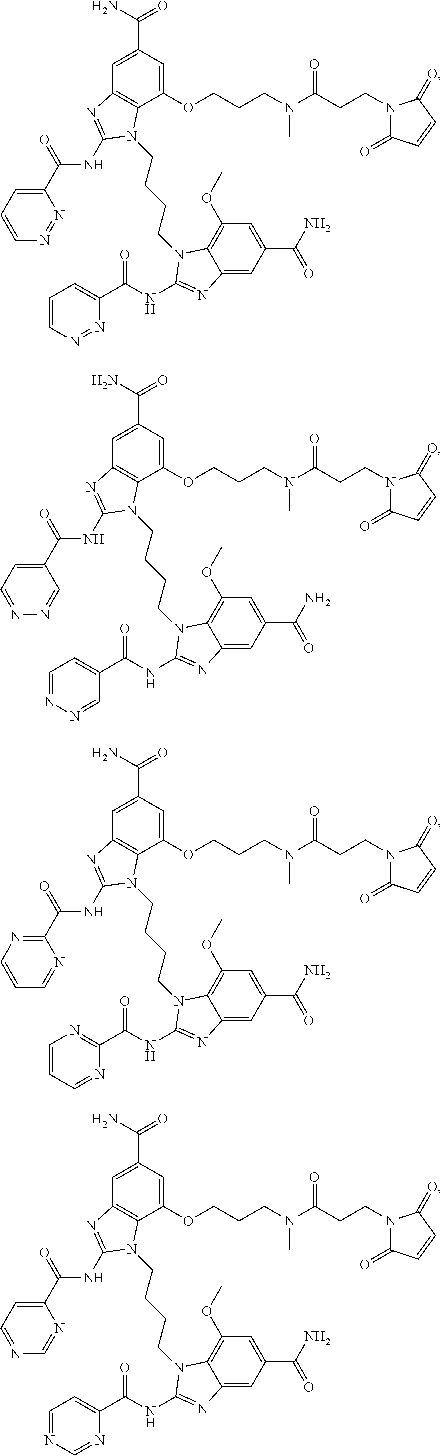 [SMILES: COc1cc(C(N)=O)cc2nc(NC(=O)c3cccnn3)n(CCCCn3c(NC(=O)c4cccnn4)nc4cc(C(N)=O)cc(OCCCN(C)C(=O)CCN5C(=O)C=CC5=O)c43)c12 ; COc1cc(C(N)=O)cc2nc(NC(=O)c3ccnnc3)n(CCCCn3c(NC(=O)c4ccnnc4)nc4cc(C(N)=O)cc(OCCCN(C)C(=O)CCN5C(=O)C=CC5=O)c43)c12 ; COc1cc(C(N)=O)cc2nc(NC(=O)c3ncccn3)n(CCCCn3c(NC(=O)c4ncccn4)nc4cc(C(N)=O)cc(OCCCN(C)C(=O)CCN5C(=O)C=CC5=O)c43)c12 ; COc1cc(C(N)=O)cc2nc(NC(=O)c3ccncn3)n(CCCCn3c(NC(=O)c4ccncn4)nc4cc(C(N)=O)cc(OCCCN(C)C(=O)CCN5C(=O)C=CC5=O)c43)c12]

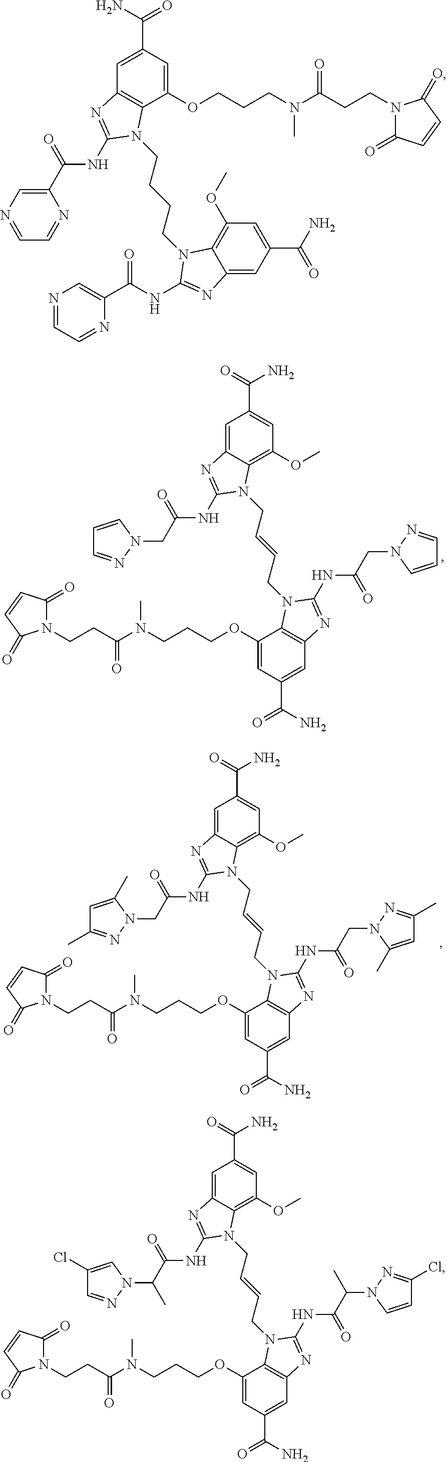

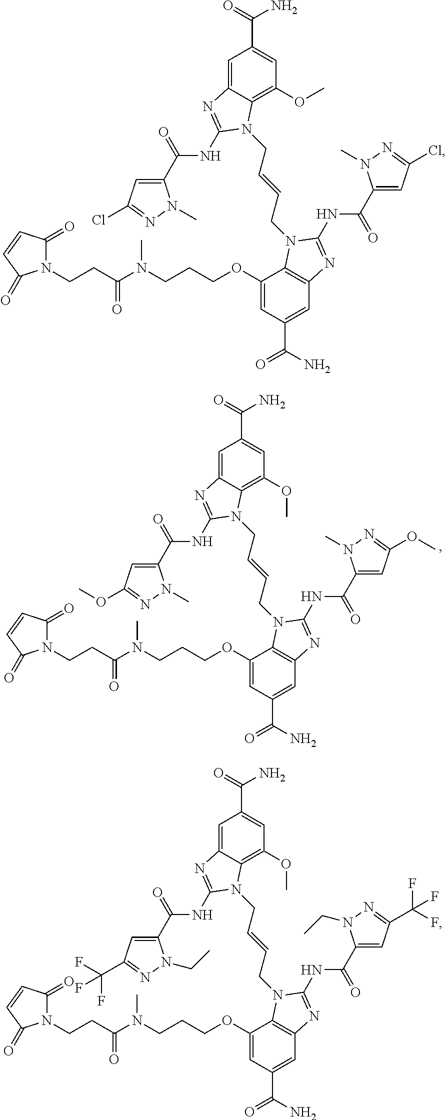

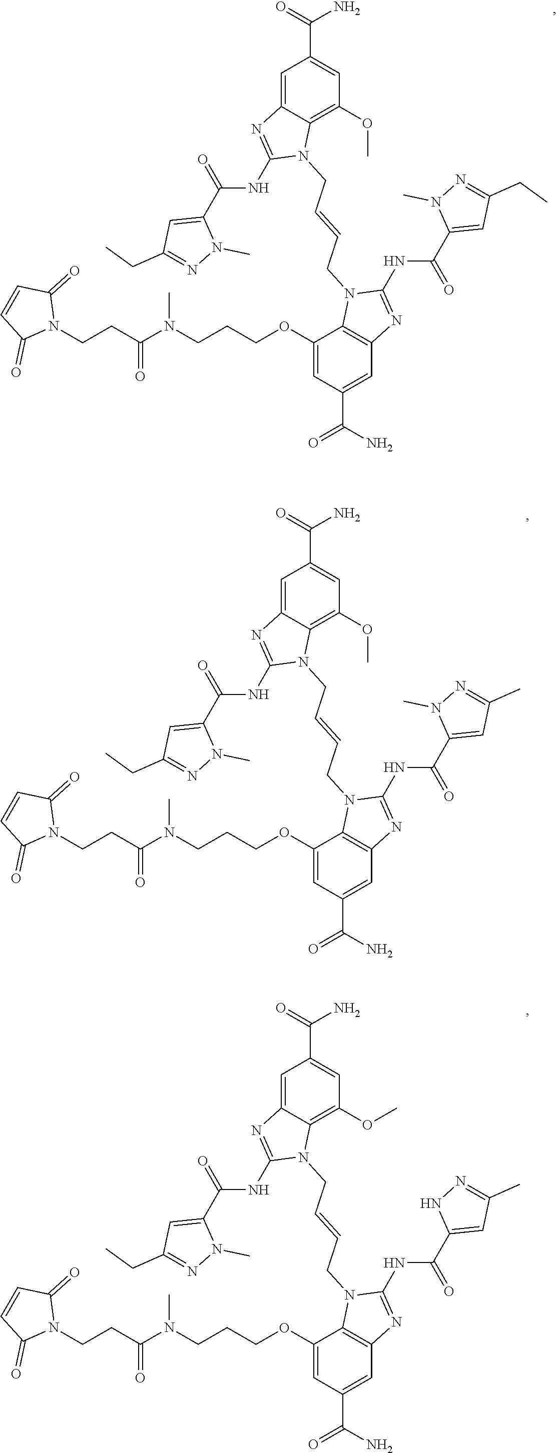 [SMILES: CCc1cc(C(=O)Nc2nc3cc(C(N)=O)cc(OC)c3n2C/C=C/Cn2c(NC(=O)c3cc(CC)nn3C)nc3cc(C(N)=O)cc(OCCCN(C)C(=O)CCN4C(=O)C=CC4=O)c32)n(C)n1 ; CCc1cc(C(=O)Nc2nc3cc(C(N)=O)cc(OC)c3n2C/C=C/Cn2c(NC(=O)c3cc(C)nn3C)nc3cc(C(N)=O)cc(OCCCN(C)C(=O)CCN4C(=O)C=CC4=O)c32)n(C)n1 ; CCc1cc(C(=O)Nc2nc3cc(C(N)=O)cc(OC)c3n2C/C=C/Cn2c(NC(=O)c3cc(C)n[nH]3)nc3cc(C(N)=O)cc(OCCCN(C)C(=O)CCN4C(=O)C=CC4=O)c32)n(C)n1]

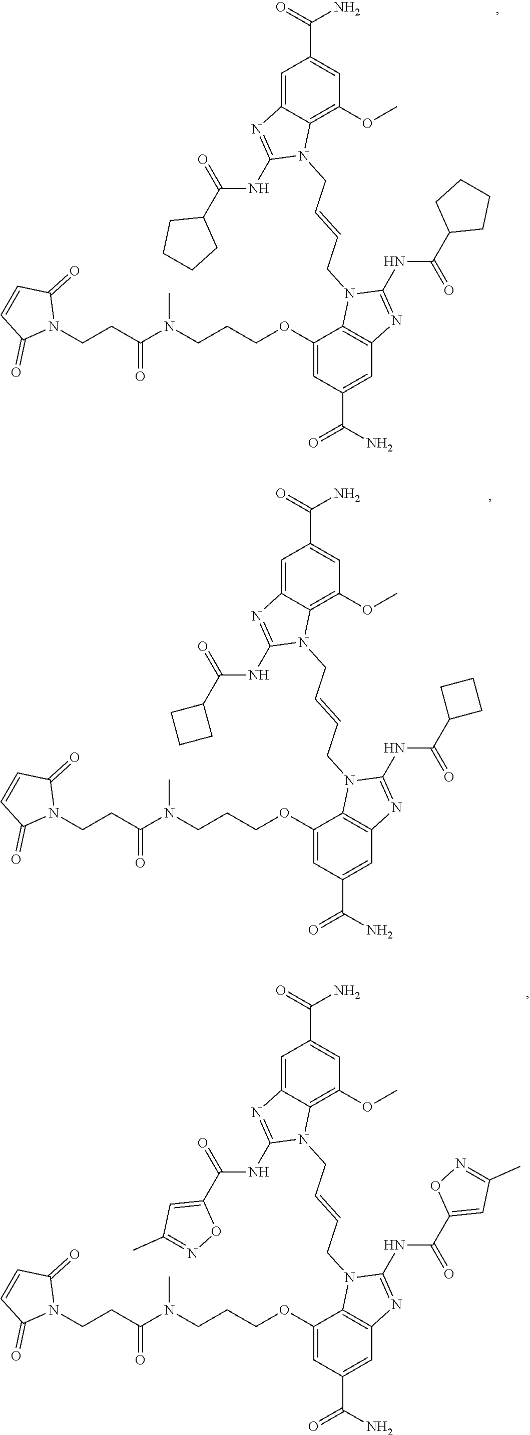 [SMILES: COc1cc(C(N)=O)cc2nc(NC(=O)C3CCCC3)n(C/C=C/Cn3c(NC(=O)C4CCCC4)nc4cc(C(N)=O)cc(OCCCN(C)C(=O)CCN5C(=O)C=CC5=O)c43)c12 ; COc1cc(C(N)=O)cc2nc(NC(=O)C3CCC3)n(C/C=C/Cn3c(NC(=O)C4CCC4)nc4cc(C(N)=O)cc(OCCCN(C)C(=O)CCN5C(=O)C=CC5=O)c43)c12 ; COc1cc(C(N)=O)cc2nc(NC(=O)c3cc(C)no3)n(C/C=C/Cn3c(NC(=O)c4cc(C)no4)nc4cc(C(N)=O)cc(OCCCN(C)C(=O)CCN5C(=O)C=CC5=O)c43)c12]

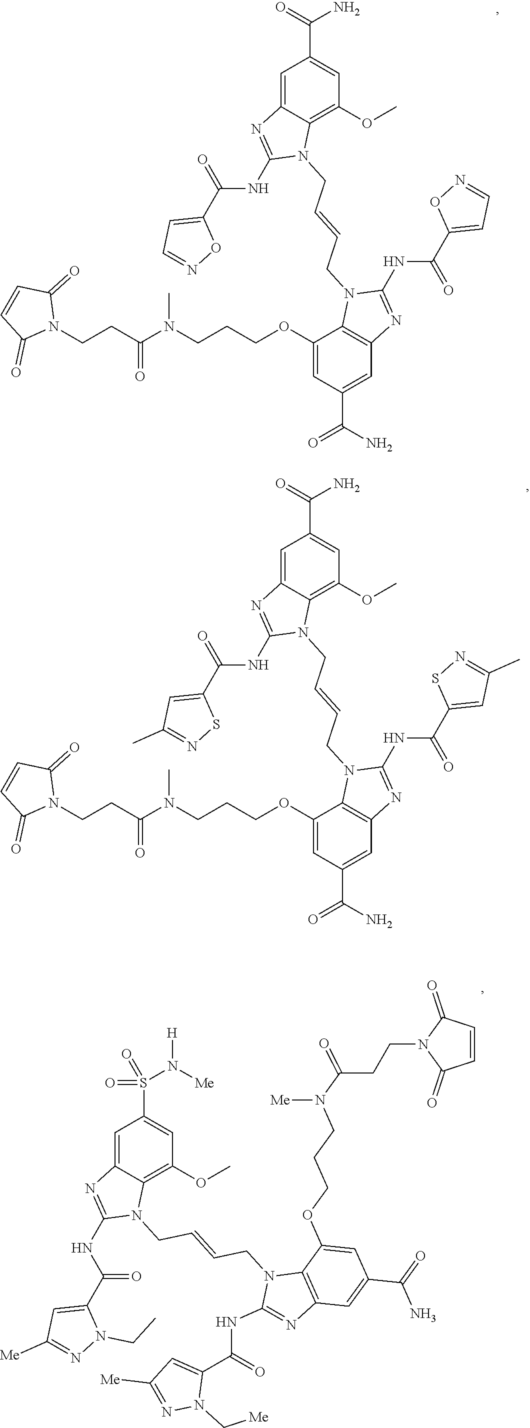 [SMILES: COc1cc(C(N)=O)cc2nc(NC(=O)c3ccno3)n(C/C=C/Cn3c(NC(=O)c4ccno4)nc4cc(C(N)=O)cc(OCCCN(C)C(=O)CCN5C(=O)C=CC5=O)c43)c12 ; COc1cc(C(N)=O)cc2nc(NC(=O)c3cc(C)ns3)n(C/C=C/Cn3c(NC(=O)c4cc(C)ns4)nc4cc(C(N)=O)cc(OCCCN(C)C(=O)CCN5C(=O)C=CC5=O)c43)c12 ; CCn1nc(C)cc1C(=O)Nc1nc2cc(S(=O)(=O)NC)cc(OC)c2n1C/C=C/Cn1c(NC(=O)c2cc(C)nn2CC)nc2cc(C(N)=O)cc(OCCCN(C)C(=O)CCN3C(=O)C=CC3=O)c21]

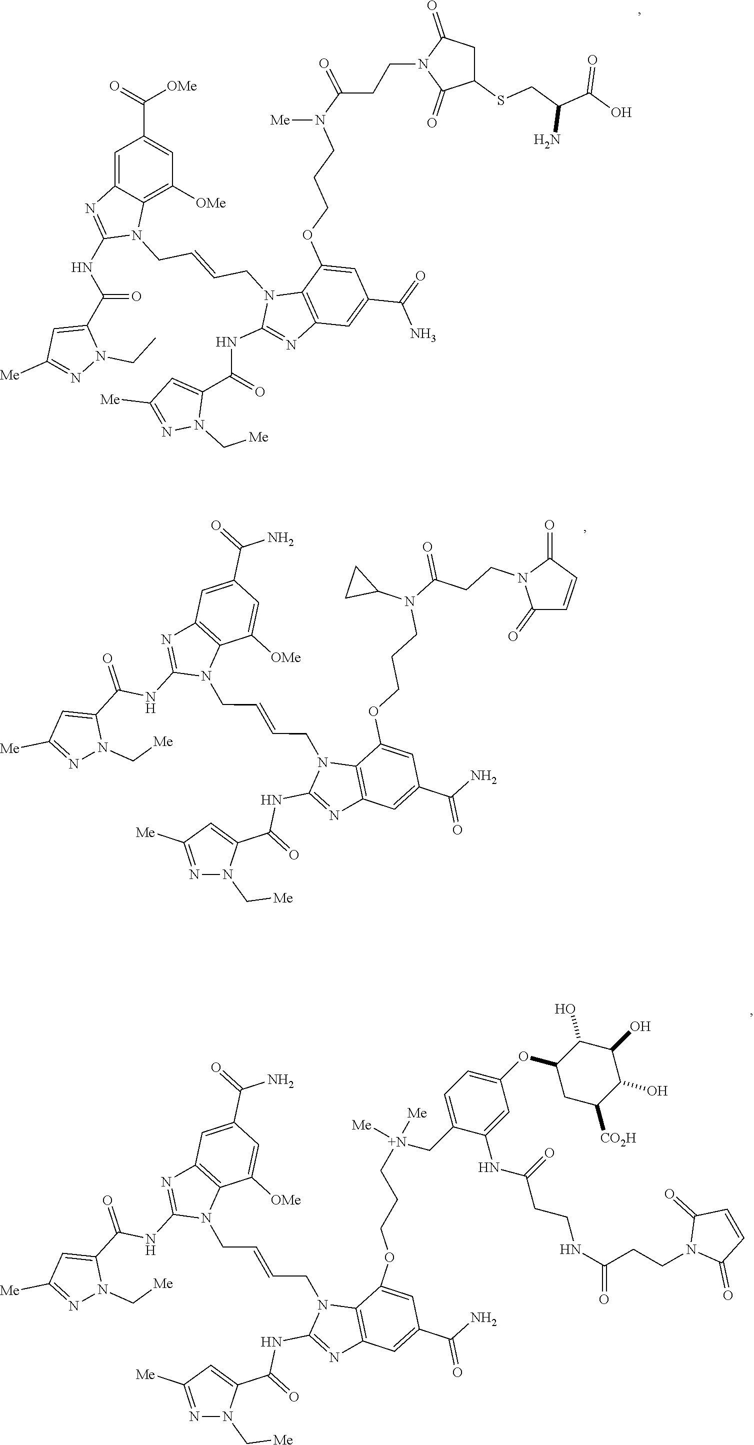

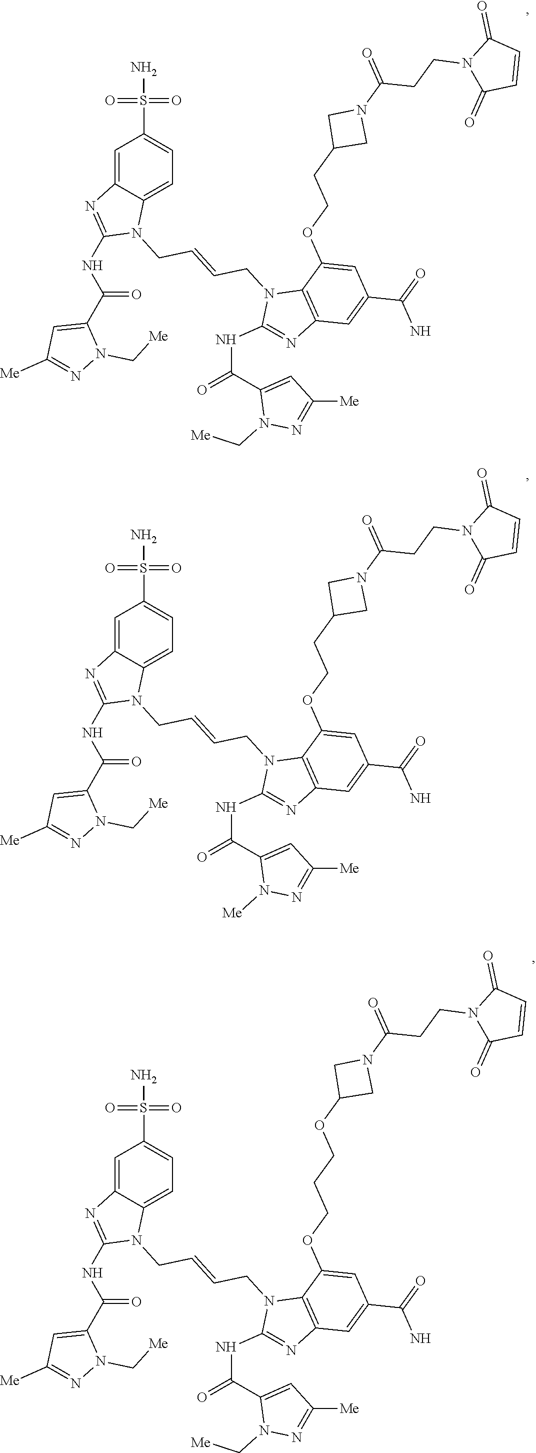 [SMILES: CCn1nc(C)cc1C(=O)Nc1nc2cc(S(N)(=O)=O)ccc2n1C/C=C/Cn1c(NC(=O)c2cc(C)nn2CC)nc2cc(C(N)=O)cc(OCCC3CN(C(=O)CCN4C(=O)C=CC4=O)C3)c21 ; CCn1nc(C)cc1C(=O)Nc1nc2cc(S(N)(=O)=O)ccc2n1C/C=C/Cn1c(NC(=O)c2cc(C)nn2C)nc2cc(C(N)=O)cc(OCCC3CN(C(=O)CCN4C(=O)C=CC4=O)C3)c21 ; CCn1nc(C)cc1C(=O)Nc1nc2cc(S(N)(=O)=O)ccc2n1C/C=C/Cn1c(NC(=O)c2cc(C)nn2CC)nc2cc(C(N)=O)cc(OCCCOC3CN(C(=O)CCN4C(=O)C=CC4=O)C3)c21]

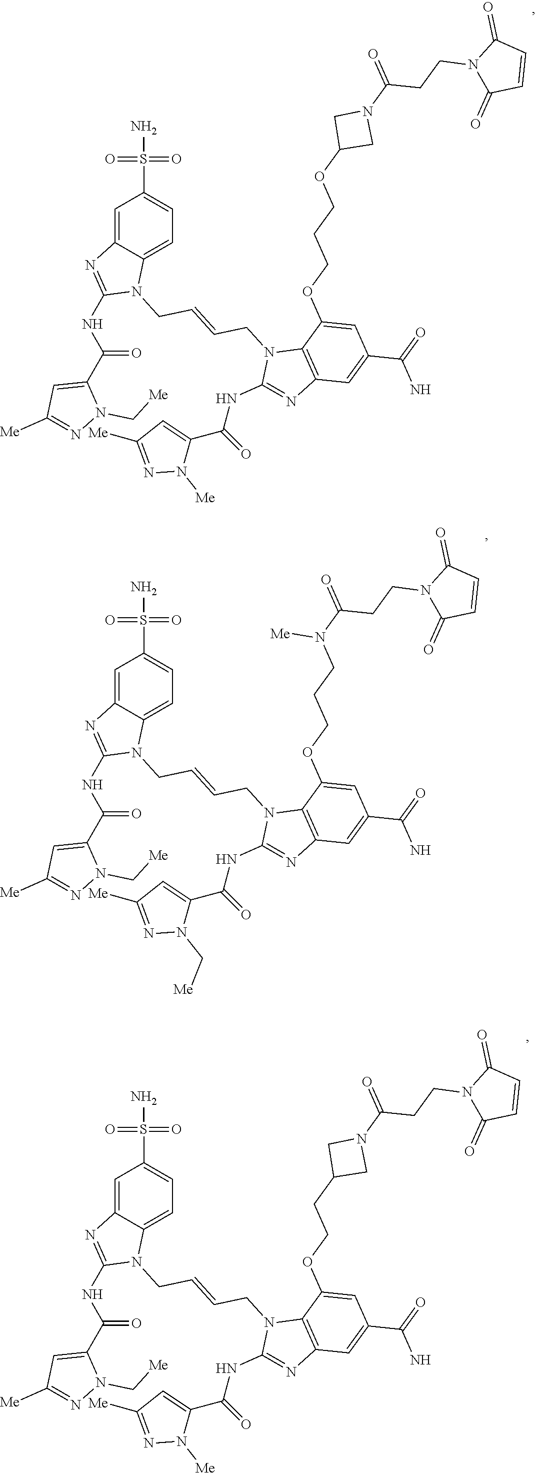 [SMILES: CCn1nc(C)cc1C(=O)Nc1nc2cc(S(N)(=O)=O)ccc2n1C/C=C/Cn1c(NC(=O)c2cc(C)nn2C)nc2cc(C(N)=O)cc(OCCCOC3CN(C(=O)CCN4C(=O)C=CC4=O)C3)c21 ; CCn1nc(C)cc1C(=O)Nc1nc2cc(S(N)(=O)=O)ccc2n1C/C=C/Cn1c(NC(=O)c2cc(C)nn2CC)nc2cc(C(N)=O)cc(OCCCN(C)C(=O)CCN3C(=O)C=CC3=O)c21 ; CCn1nc(C)cc1C(=O)Nc1nc2cc(S(N)(=O)=O)ccc2n1C/C=C/Cn1c(NC(=O)c2cc(C)nn2C)nc2cc(C(N)=O)cc(OCCC3CN(C(=O)CCN4C(=O)C=CC4=O)C3)c21]

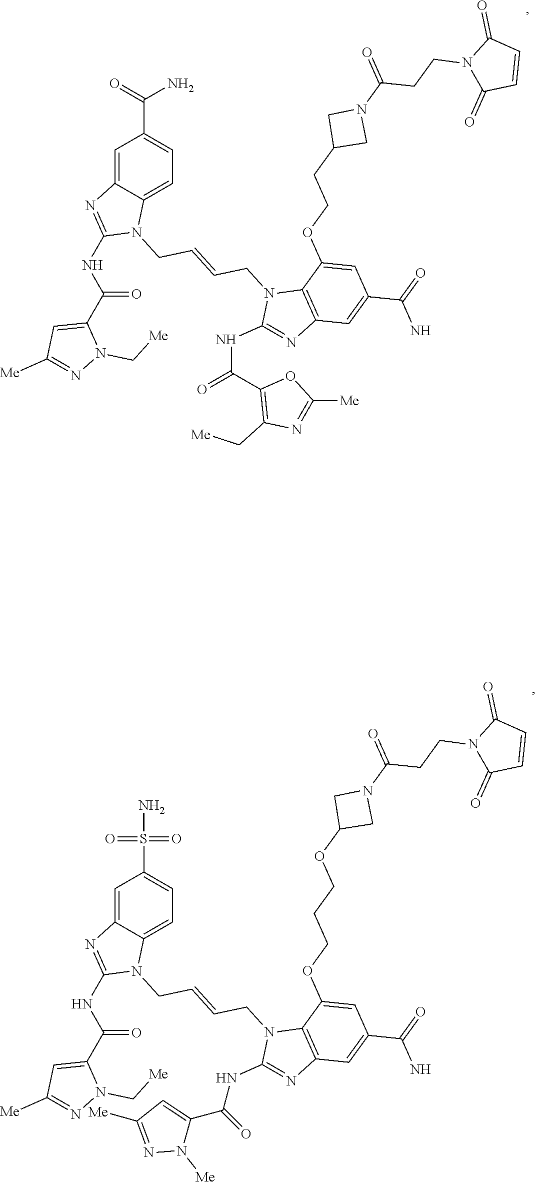 [SMILES: CCc1nc(C)oc1C(=O)Nc1nc2cc(C(N)=O)cc(OCCC3CN(C(=O)CCN4C(=O)C=CC4=O)C3)c2n1C/C=C/Cn1c(NC(=O)c2cc(C)nn2CC)nc2cc(C(N)=O)ccc21 ; CCn1nc(C)cc1C(=O)Nc1nc2cc(S(N)(=O)=O)ccc2n1C/C=C/Cn1c(NC(=O)c2cc(C)nn2C)nc2cc(C(N)=O)cc(OCCCOC3CN(C(=O)CCN4C(=O)C=CC4=O)C3)c21]

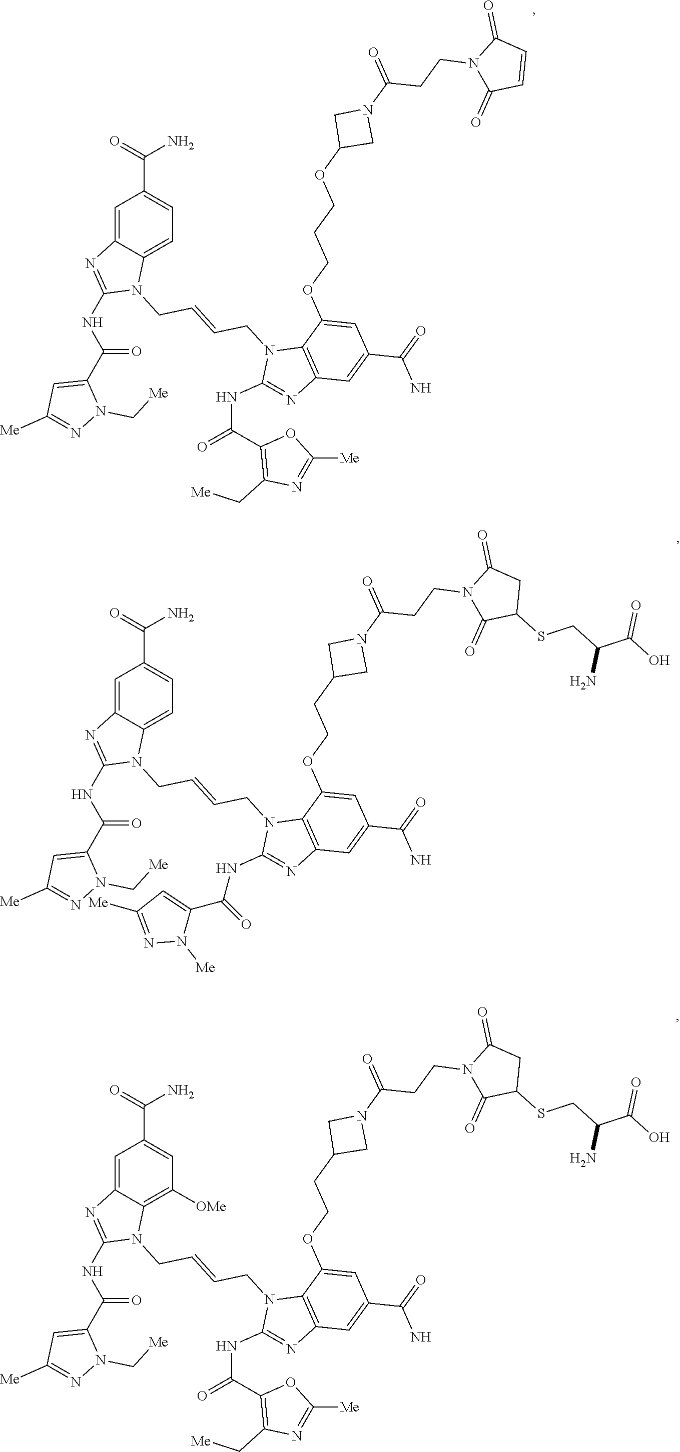 [SMILES: CCc1nc(C)oc1C(=O)Nc1nc2cc(C(N)=O)cc(OCCCOC3CN(C(=O)CCN4C(=O)C=CC4=O)C3)c2n1C/C=C/Cn1c(NC(=O)c2cc(C)nn2CC)nc2cc(C(N)=O)ccc21 ; CCn1nc(C)cc1C(=O)Nc1nc2cc(C(N)=O)ccc2n1C/C=C/Cn1c(NC(=O)c2cc(C)nn2C)nc2cc(C(N)=O)cc(OCCC3CN(C(=O)CCN4C(=O)CC(SC[C@H](N)C(=O)O)C4=O)C3)c21 ; CCc1nc(C)oc1C(=O)Nc1nc2cc(C(N)=O)cc(OCCC3CN(C(=O)CCN4C(=O)CC(SC[C@H](N)C(=O)O)C4=O)C3)c2n1C/C=C/Cn1c(NC(=O)c2cc(C)nn2CC)nc2cc(C(N)=O)cc(OC)c21]

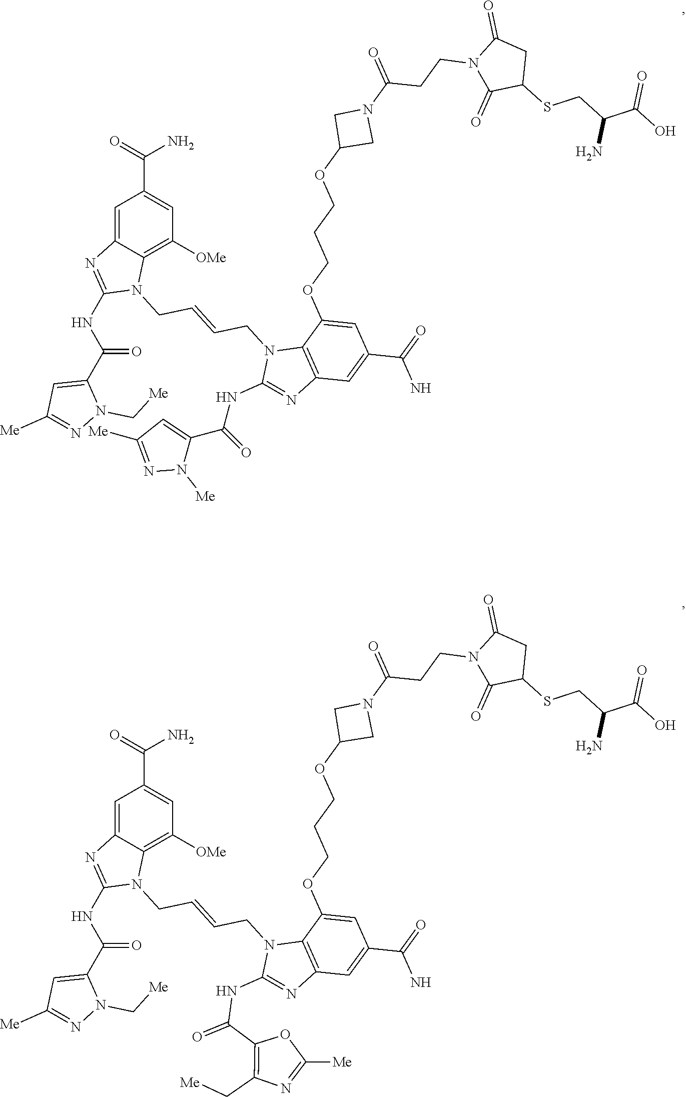 [SMILES: CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCCOC3CN(C(=O)CCN4C(=O)CC(SC[C@H](N)C(=O)O)C4=O)C3)c21 ; CCc1nc(C)oc1C(=O)Nc1nc2cc(C(N)=O)cc(OCCCOC3CN(C(=O)CCN4C(=O)CC(SC[C@H](N)C(=O)O)C4=O)C3)c2n1C/C=C/Cn1c(NC(=O)c2cc(C)nn2CC)nc2cc(C(N)=O)cc(OC)c21]

and pharmaceutically acceptable salts thereof.

### Compounds of Formula (V)

Some embodiments include a compound of Formula (V):

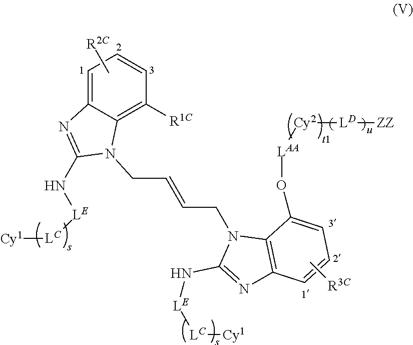

or a pharmaceutically acceptable salt thereof, wherein:
R^{1C} is hydrogen, hydroxyl, C₁₋₆ alkoxy, —(C₁₋₆ alkyl) C₁₋₆ alkoxy, —(CH₂)ₙ—NR^{A}R^{B}, or PEG2 to PEG4;
R^{2C} is —CO₂R^{M}, —(C═O)NR^{C}R^{D}, S(O)₂NR^{C}R^{D}, —S(O)₂R^{M}, —(CH₂)_{q}—NR^{E}R^{F}, —(CH₂)_{q}—OR^{M}, —O(C═O)—NR^{E}W, or —NR^{M}(C═O)—NR^{E}R^{F}, wherein R^{2C} is attached at any one of positions labeled 1, 2, or 3;
R^{3C} is —CO₂R^{M}, —(C═O)NR^{C}R^{D}, —S(O)₂NR^{C}R^{D}, —S(O)₂R^{M}, —(CH₂)_{q}—NR^{E}R^{F}, —(CH₂)_{q}—OR^{M}, —O(C═O)—NR^{E}W, or —NR^{M}(C═O)—NR^{E}R^{F}, wherein R^{3C} is attached at any one of positions labeled 1′, 2′, or 3′;
each R^{A}, R^{B}, R^{C}, R^{D}, R^{E}, R^{F}, and R^{M} are independently hydrogen or C₁₋₆ alkyl;
each subscript n is independently an integer from 0 to 6;
each subscript q is independently an integer from 0 to 6;
L^{E} is —(C═O)— or —S(O)₂—;
L^{C} is —(CR^{I}R^{J})₁₋₃—
each R^{I} and R^{J} are independently hydrogen or C₁₋₃ alkyl;
subscript s is 0 or 1;
each Cy¹ is independently a 4-6 membered heterocycle, a 5-6 membered heteroaryl, or a C₃₋₆ cycloalkyl, each optionally substituted with one or more R^{K};
each R^{K} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d2}R^{e2}, —C(O)NR^{d2}R^{e2}, —C(O)(C₁₋₆ alkyl), and —C(O)O(C₁₋₆ alkyl);
each R^{d2} and R^{e2} are independently hydrogen or C₁₋₃ alkyl;
L^{AA} is —(CH₂)₁₋₆—, —C(O)(CH₂)₁₋₆—, —C(O)NR^{L}(CH₂)₁₋₆—, —(CH₂)₁₋₆O—, —C(O)(CH₂)₁₋₆O—, or —C(O)NR^{L}(CH₂)₁₋₆O—;
R^{L} is hydrogen or C₁₋₃ alkyl;
Cy² is C₃₋₆ cycloalkyl, 4-6 membered heterocycle, 5-6 membered heteroaryl, or phenyl, each optionally substituted with one or more R^{U};
each R^{U} is independently selected from the group consisting of —CO₂R^{j1}, —(C═O)NR^{d3}R^{e3}, —S(O)₂NR^{d3}R^{e3}, —(CH₂)_{q1}—NR^{g1}R^{h1}, —(CH₂)_{q1}—OR^{j1}, and —(CH₂)_{q1}—(OCH₂CH₂)₁₋₈OH;
each R^{d3}, R^{e3}, R^{g1}, R^{h1}, and R^{j1} are independently hydrogen or C₁₋₆ alkyl;
subscript q1 is an integer from 0 to 6;
subscript t1 is 0 or 1;
L^{D} is —(CH₂)₁₋₆—;
subscript u is 0 or 1;
when t1 is 0, ZZ is —NR^{Q}R^{R}, —N⁺(C₁₋₆ alkyl)R^{Q}R^{R}, —C(═O)N^{S}R^{T}, —C(O)O(C₁₋₆ alkyl), —CO₂H, or an amino acid, or when t1 is 1, ZZ is hydrogen, —NR^{Q}R^{R}, —N⁺(C₁₋₆ alkyl)R^{Q}R^{R}; —C(═O)N^{S}R^{T}, —C(O)O(C₁₋₆ alkyl), —CO₂H, or an amino acid;
R^{Q} is hydrogen, C₁₋₆ alkyl, C₃₋₆ cycloalkyl, —(CH₂)₁₋₃C₃₋₆ cycloalkyl, —(CH₂)₁₋₃C₁₋₃ alkoxy, —(CH₂)₁₋₃ 4-6 membered heterocycle, or —(CH₂)₁₋₃ 5-6 membered heteroaryl, provided that
   if t1 is 0 and both Cy¹ are

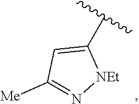

then R^{Q} is C₂₋₆ alkyl, C₃₋₆ cycloalkyl, —(CH₂)₁₋₃C₃₋₆ cycloalkyl, —(CH₂)₁₋₃C₁₋₃ alkoxy, —(CH₂)₁₋₃ 4-6 membered heterocycle, or —(CH₂)₁₋₃ 5-6 membered heteroaryl, and
if t1 is 0 and at least one Cy¹ is not

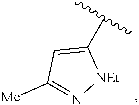

then ZZ is —NR^{Q}R^{R}, —N⁺(C₁₋₆ alkyl)R^{Q}R^{R}, or —C(═O)N^{S}R^{T}, and R^{Q} is C₁₋₆ alkyl, C3.6 cycloalkyl, —(CH₂)₁₋₃C₃₋₆ cycloalkyl, —(CH₂)₁₋₃C₁₋₃ alkoxy, —(CH₂)₁₋₃ 4-6 membered heterocycle, or —(CH₂)₁₋₃ 5-6 membered heteroaryl; and
each R^{R}, R^{S}, and R^{T} are independently hydrogen or C₁₋₆ alkyl.

In some embodiments, R^{1C} is hydrogen. In some embodiments, R^{1C} is hydroxyl. In some embodiments, R^{1C} is C₁₋₆ alkoxy. In some embodiments, R^{1C} is methoxy. In some embodiments, R^{1C} is —(C₁₋₆ alkyl)C₁₋₆ alkoxy. In some embodiments, R^{1C} is methoxyethyl. In some embodiments, R^{1C} is PEG2 to PEG4. In some embodiments, R^{1C} is —(CH₂)ₙ—NR^{A}R^{B}. In some embodiments, R^{A} and R^{B} are both hydrogen. In some embodiments, R^{A} and R^{B} are independently C₁₋₃ alkyl. In some embodiments, one of R^{A} and R^{B} is hydrogen and the other of R^{A} and R^{B} is C₁₋₃ alkyl. In some embodiments, each subscript n is 0. In some embodiments, each subscript n is 1. In some embodiments, each subscript n is 2. In some embodiments, each subscript n is 3, 4, 5, or 6.

In some embodiments, R^{2C} and R^{3C} are —CO₂H, —(C═O)ₘ—NR^{C}R^{D}, or —(CH₂)_{q}—NR^{E}R^{F}; and R^{2C} and R^{3C} are the same. In some embodiments, R^{2C} and R^{3C} are independently —CO₂H, —(C═O)ₘ—NR^{C}R^{D}, or —(CH₂)_{q}—NR^{E}R^{F}; and R^{2C} and R^{3C} are different.

In some embodiments, R^{2C} is —(C═O)ₘ—NR^{C}R^{D}. In some embodiments, R^{3C} is —(C═O)ₘ—NR^{C}R^{D}. In some embodiments, R^{C} and R^{D} are both hydrogen. In some embodiments, R^{C} and R^{D} are each independently C₁₋₃ alkyl. In some embodiments, one of R^{C} and R^{D} is hydrogen and the other of R^{C} and R^{D} is C₁₋₃ alkyl. In some embodiments, each subscript m is 0. In some embodiments, each subscript m is 1.

In some embodiments, R^{2C} is —(CH₂)_{q}—NR^{E}R^{F}. In some embodiments, R^{3C} is —(CH₂)_{q}—NR^{E}R^{F}. In some embodiments, R^{E} and R^{F} are both hydrogen. In some embodiments, R^{E} and R^{F} are each independently C₁₋₃ alkyl. In some embodiments, one of R^{E} and R^{F} is hydrogen and the other of R^{E} and R^{F} is C₁₋₃ alkyl.

In some embodiments, each subscript q is 0. In some embodiments, each subscript q is an integer from 1 to 6.

In some embodiments, R^{2C} is —CO₂R^{M}. In some embodiments, R^{3C} is —CO₂R^{M}.

In some embodiments, R^{M} is hydrogen. In some embodiments, R^{M} is C₁₋₃ alkyl.

In some embodiments, R^{2C} is —(CH₂)_{q}—OR^{M}. In some embodiments, R^{3C} is —(CH₂)_{q}—OR^{M}.

In some embodiments, R^{M} is hydrogen. In some embodiments, subscript q is 0. In some embodiments, subscript q is 1.

In some embodiments, R^{2C} is —O(C═O)—NR^{E}R^{F}. In some embodiments, R^{3C} is —O(C═O)—NR^{E}R^{F}. In some embodiments, R^{E} and R^{F} are both hydrogen. In some embodiments, R^{E} and R^{F} are each independently C₁₋₃ alkyl. In some embodiments, one of R^{E} and R^{F} is hydrogen and the other of R^{E} and R^{F} is C₁₋₃ alkyl.

In some embodiments, R^{2C} is —NR^{M}(C═O)—NR^{E}R^{F}. In some embodiments, R^{3C} is —NR^{M}(C═O)—NR^{E}R^{F}. In some embodiments, R^{E}, R^{F}, and R^{M} are all hydrogen. In some embodiments, R^{E}, R^{F}, and R^{M} are each independently C₁₋₃ alkyl. In some embodiments, one of R^{E}, R^{F}, and R^{M} is C₁₋₃ alkyl and the rest of R^{E}, R^{F}, and R^{M} is hydrogen.

In some embodiments, R^{2C} is —S(O)₂NR^{C}R^{D}.

In some embodiments, R^{3C} is —S(O)₂NR^{C}R^{D}. In some embodiments, R^{C} and R^{D} are both hydrogen. In some embodiments, R^{C} and R^{D} are each independently C₁₋₃ alkyl. In some embodiments, one of R^{C} and R^{D} is hydrogen and the other of R^{C} and R^{D} is C₁₋₃ alkyl.

In some embodiments, R^{2C} is —S(O)₂R^{M}. In some embodiments, R^{3C} is —S(O)₂R^{M}. In some embodiments, R^{M} is hydrogen. In some embodiments, R^{M} is C₁₋₃ alkyl.

In some embodiments, R^{2C} is attached at position 1. In some embodiments, R^{2C} is attached at position 2. In some embodiments, R^{2C} is attached at position 3. In some embodiments, R^{3C} is attached at position 1′. In some embodiments, R^{3C} is attached at position 2′. In some embodiments, R^{3C} is attached at position 3′.

In some embodiments, L^{E} is —(C═O)—. In some embodiments L^{E} is —S(O)₂—.

In some embodiments, each R^{I} and R^{J} is hydrogen. In some embodiments, each R^{I} and R^{J} is C₁₋₃ alkyl. In some embodiments, one of R^{I} and R^{J} is hydrogen and the other of R^{I} and R^{J} is C₁₋₃ alkyl.

In some embodiments, L^{C} is —(CR^{I}R^{J})—.

In some embodiments, subscript s is 0. In some embodiments, subscript s is 1.

In some embodiments, each Cy¹ is independently a 5-6 membered heteroaryl. In some embodiments, each Cy¹ is pyrazole optionally substituted with one or more R^{K}. In some embodiments, each Cy¹ is independently selected from the group consisting of pyrazole, imidazole, furan, thiophene, thiazole, isothiazole, oxazole, isoxazole, pyrrole, pyridazine, pyridine, pyrimidine, and pyrazine, each optionally substituted with one or more R^{K}. In some embodiments, each Cy¹ is independently selected from the group consisting of imidazole, furan, thiophene, thiazole, isothiazole, oxazole, isoxazole, pyrrole, pyridazine, pyridine, pyrimidine, and pyrazine, each optionally substituted with one or more R^{K}. In some embodiments, each Cy¹ is independently a C4-s cycloalkyl optionally substituted with one or more R^{K}. In some embodiments, each R^{K} is independently selected from the group consisting of C In some embodiments, each R^{K} is independently selected from the group consisting of methyl, ethyl, —CF₃, and halogen.

In some embodiments, each Cy¹ is the same. In some embodiments, each Cy¹ is different.

In some embodiments, L^{AA} is —(CH₂)₁₋₆—. In some embodiments, L^{AA} is —(CH₂)₁₋₃—. In some embodiments, L^{AA} is —(CH₂)₁₋₆O—. In some embodiments, L^{AA} is —(CH₂)₁₋₃O—.

In some embodiments, Cy² is a 4-6 membered heterocycle. In some embodiments, Cy has the structure:

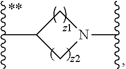

wherein each of subscripts z1 and z2 is independently an integer from 1 to 3 and ** indicates attachment to L^{AA}.

In some embodiments, subscript z1 and subscript z2 are 1. In some embodiments, subscript z1 and subscript z2 are 2.

In some embodiments, subscript z1 is 1 and subscript z2 is 2.

In some embodiments, Cy² has the structure:

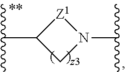

wherein
Z¹ is selected from the group consisting of —O—, —S—, —CR^{N}R^{O}—, and —NR^{P}—;
R^{N}, R^{O}, and R^{P} are independently hydrogen or C₁₋₆ alkyl;
subscript z3 is an integer from 1 to 3; and
** indicates attachment to L^{AA}.

In some embodiments, R^{N} and R^{O} are hydrogen. In some embodiments, R^{P} is hydrogen. In some embodiments, R^{P} is methyl.

In some embodiments, Cy² is a 5-6 membered heteroaryl.

In some embodiments, Cy² is selected from the group consisting of:

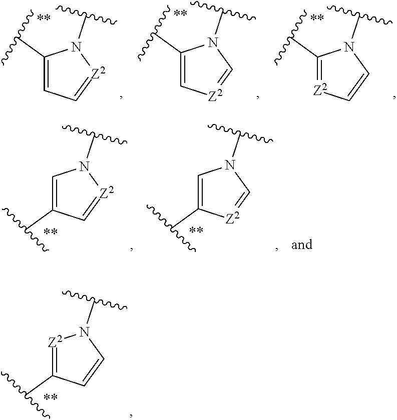

wherein
Z² is ═CR^{N}— or ═N—;
R^{N} is hydrogen or C₁₋₆ alkyl; and
** indicates attachment to L^{AA}.

In some embodiments, Z² is ═CR^{N}— and R^{N} is hydrogen. In some embodiments, Z² is ═N—.

In some embodiments, Cy² is selected from the group consisting of:

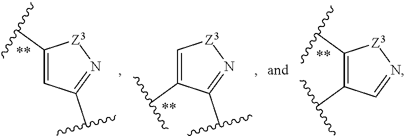

wherein Z³ is —O— or —S— and ** indicates attachment to L^{AA}, L^{D}, NR^{HH}, Y, W, or L^{BB}.

In some embodiments, ** indicates attachment to L^{AA}. In some embodiments, ** indicates attachment to L^{D}, NR^{HH}, Y, W, or L^{BB}.

In some embodiments, Cy² is selected from the group consisting of:

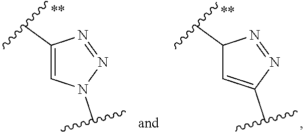 [SMILES: *c1cn(*)nn1 ; *C1=CC(*)N=N1]

wherein ** indicates attachment to L^{AA}.

In some embodiments, Cy² is selected from the group consisting of:

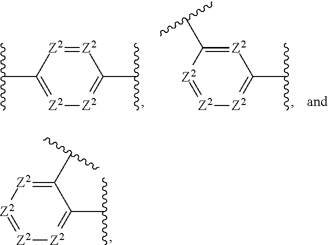

wherein
each Z² is independently ═CR^{N}— or ═N—; and
each R^{N} is hydrogen or C₁₋₆ alkyl.

In some embodiments, at least one Z² is ═N—. In some embodiments, one Z² is ═N— and the remaining Z² are ═CR^{N}—. In some embodiments, two Z² are —NR^{P}— and the remaining Z2 are ═CR^{N}.

In some embodiments, R^{N} is hydrogen.

In some embodiments, Cy² is selected from the group consisting of:

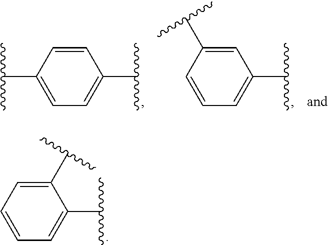 [SMILES: *c1ccc(*)cc1 ; *c1cccc(*)c1 ; *c1ccccc1*]

In some embodiments, Cy² is cyclobutyl.

In some embodiments, R^{d3}, R^{e3}, R^{g1}, R^{h1}, and R^{j1} are independently hydrogen or —CH₃.

In some embodiments, ach R^{U} is independently selected from —CO₂H, —(C═O)NH₂, —S(O)₂NH₂, —CH₂NH₂, and —CH₂OH.

In some embodiments, t1 is 0. In some embodiments, t1 is 1.

In some embodiments, u is 1 and L^{D} is —(CH₂)₁₋₃. In some embodiments, u is 0.

In some embodiments, ZZ is —NR^{Q}R^{R}. In some embodiments, R^{Q} is C₁₋₆ alkyl, In some embodiments, R^{Q} is C3.6 cycloalkyl. In some embodiments, R^{Q} is cyclopropyl. In some embodiments, R^{Q} is —(CH₂)₁₋₃C₃₋₆ cycloalkyl. In some embodiments, R^{R} is hydrogen.

In some embodiments, ZZ is —N⁺(C₁₋₆ alkyl)R^{Q}R^{R}.

In some embodiments, ZZ is —C(═O)N^{S}R^{T}.

In some embodiments, ZZ is —C(O)O(t-butyl).

In some embodiments, ZZ is —CO₂H.

In some embodiments, ZZ is an amino acid selected from the group consisting of alanine, valine, isoleucine, leucine, aspartic acid, glutamic acid, lysine, histidine, arginine, glycine, serine, threonine, phenylalanine, O-methylserine, O-methylaspartic acid, O-methylglutamic acid, N-methyllysine, O-methyltyrosine, O-methylhistidine, and O-methylthreonine.

Some embodiments of Formula (V) include compounds selected from the group consisting of:

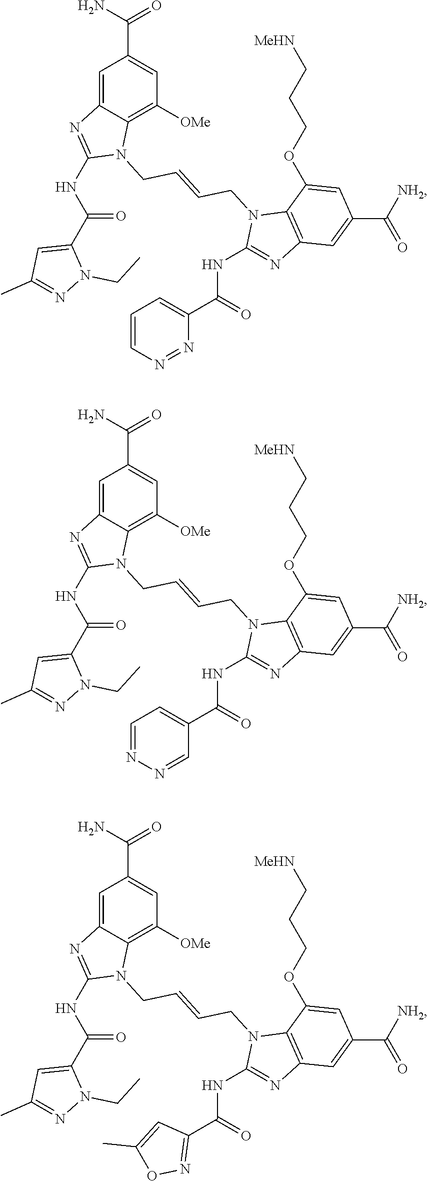 [SMILES: CCn1nc(C)cc1C(=O)Nc1nc2cc(C(N)=O)cc(OC)c2n1C/C=C/Cn1c(NC(=O)c2cccnn2)nc2cc(C(N)=O)cc(OCCCNC)c21 ; CCn1nc(C)cc1C(=O)Nc1nc2cc(C(N)=O)cc(OC)c2n1C/C=C/Cn1c(NC(=O)c2ccnnc2)nc2cc(C(N)=O)cc(OCCCNC)c21 ; CCn1nc(C)cc1C(=O)Nc1nc2cc(C(N)=O)cc(OC)c2n1C/C=C/Cn1c(NC(=O)c2cc(C)on2)nc2cc(C(N)=O)cc(OCCCNC)c21]

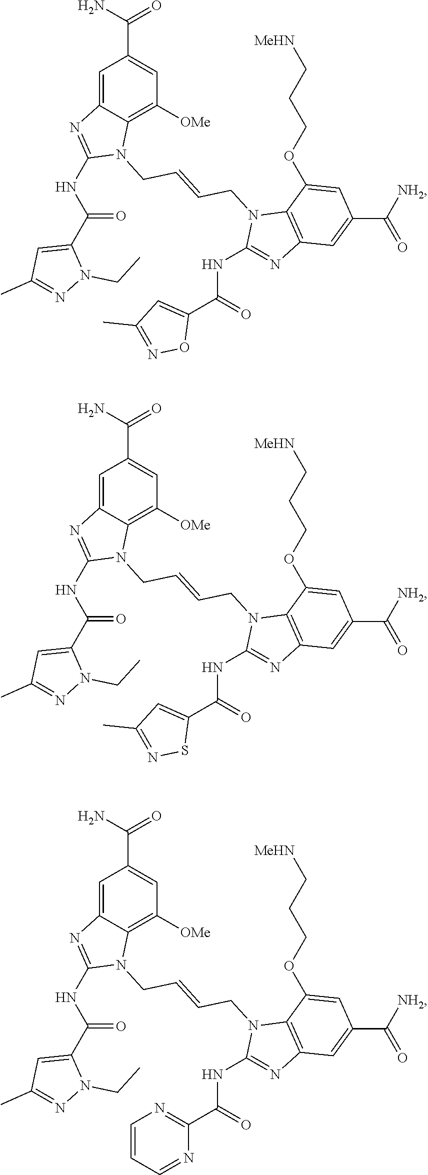 [SMILES: CCn1nc(C)cc1C(=O)Nc1nc2cc(C(N)=O)cc(OC)c2n1C/C=C/Cn1c(NC(=O)c2cc(C)no2)nc2cc(C(N)=O)cc(OCCCNC)c21 ; CCn1nc(C)cc1C(=O)Nc1nc2cc(C(N)=O)cc(OC)c2n1C/C=C/Cn1c(NC(=O)c2cc(C)ns2)nc2cc(C(N)=O)cc(OCCCNC)c21 ; CCn1nc(C)cc1C(=O)Nc1nc2cc(C(N)=O)cc(OC)c2n1C/C=C/Cn1c(NC(=O)c2ncccn2)nc2cc(C(N)=O)cc(OCCCNC)c21]

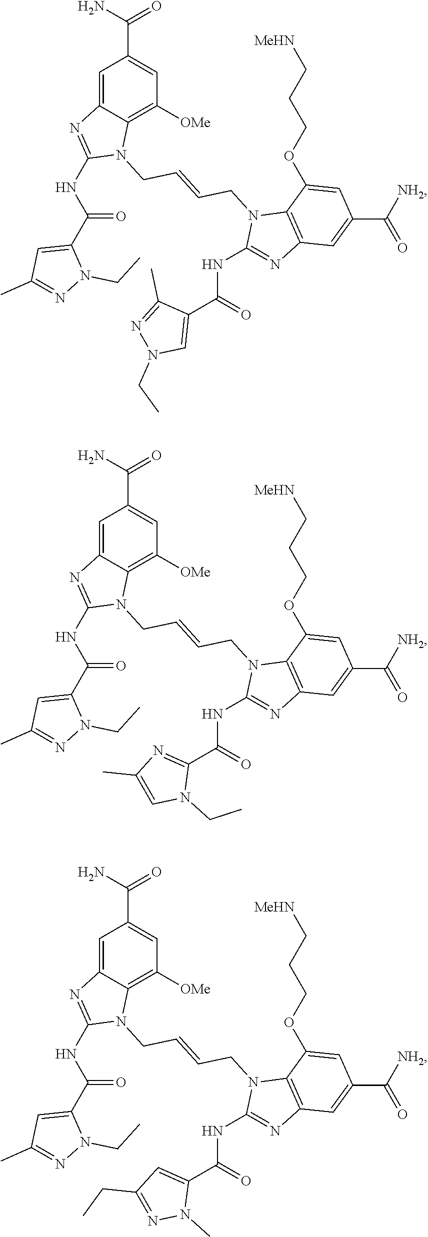

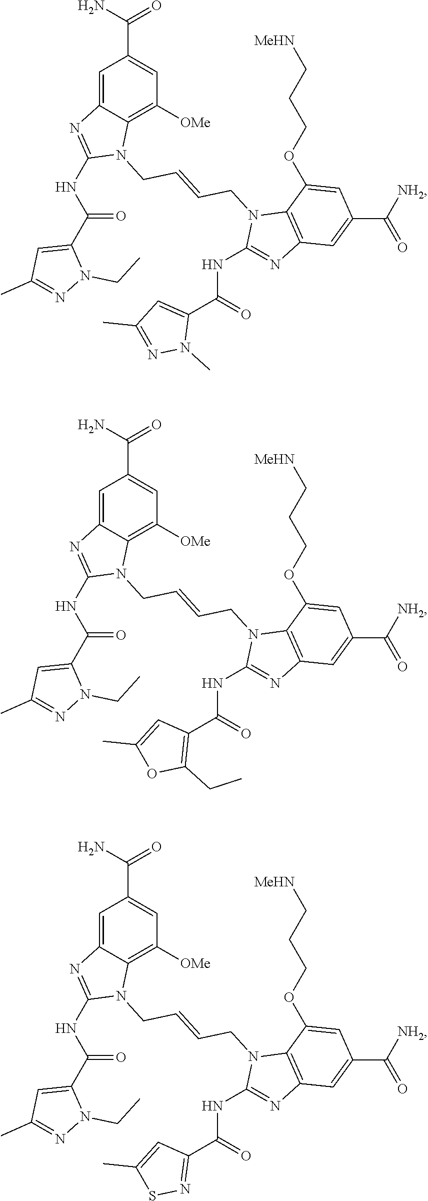 [SMILES: CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCCNC)c21 ; CCc1oc(C)cc1C(=O)Nc1nc2cc(C(N)=O)cc(OCCCNC)c2n1C/C=C/Cn1c(NC(=O)c2cc(C)nn2CC)nc2cc(C(N)=O)cc(OC)c21 ; CCn1nc(C)cc1C(=O)Nc1nc2cc(C(N)=O)cc(OC)c2n1C/C=C/Cn1c(NC(=O)c2cc(C)sn2)nc2cc(C(N)=O)cc(OCCCNC)c21]

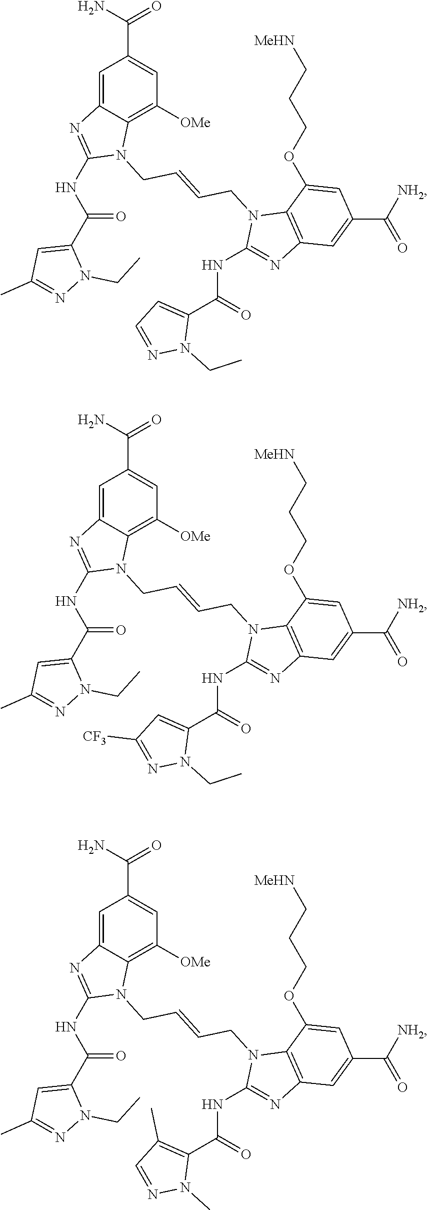 [SMILES: CCn1nc(C)cc1C(=O)Nc1nc2cc(C(N)=O)cc(OC)c2n1C/C=C/Cn1c(NC(=O)c2ccnn2CC)nc2cc(C(N)=O)cc(OCCCNC)c21 ; CCn1nc(C)cc1C(=O)Nc1nc2cc(C(N)=O)cc(OC)c2n1C/C=C/Cn1c(NC(=O)c2cc(C(F)(F)F)nn2CC)nc2cc(C(N)=O)cc(OCCCNC)c21 ; CCn1nc(C)cc1C(=O)Nc1nc2cc(C(N)=O)cc(OC)c2n1C/C=C/Cn1c(NC(=O)c2c(C)cnn2C)nc2cc(C(N)=O)cc(OCCCNC)c21]

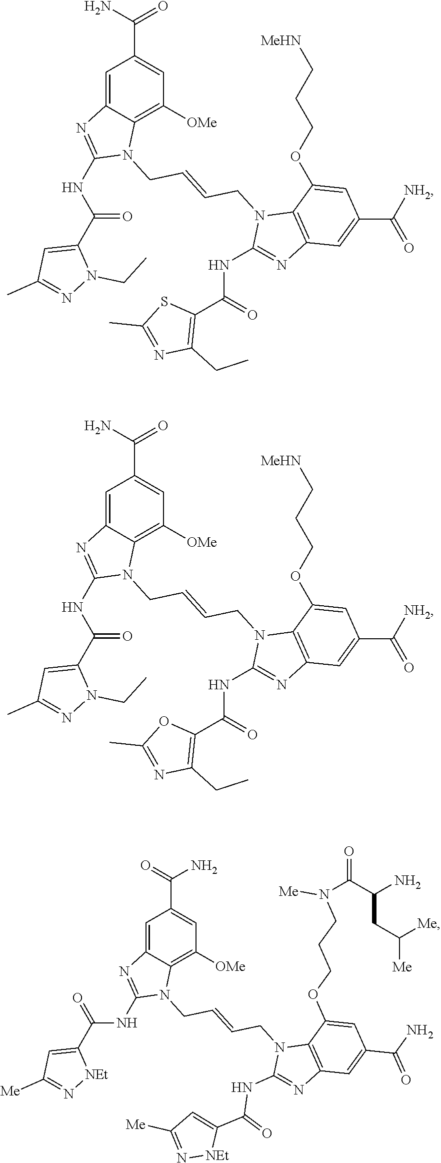

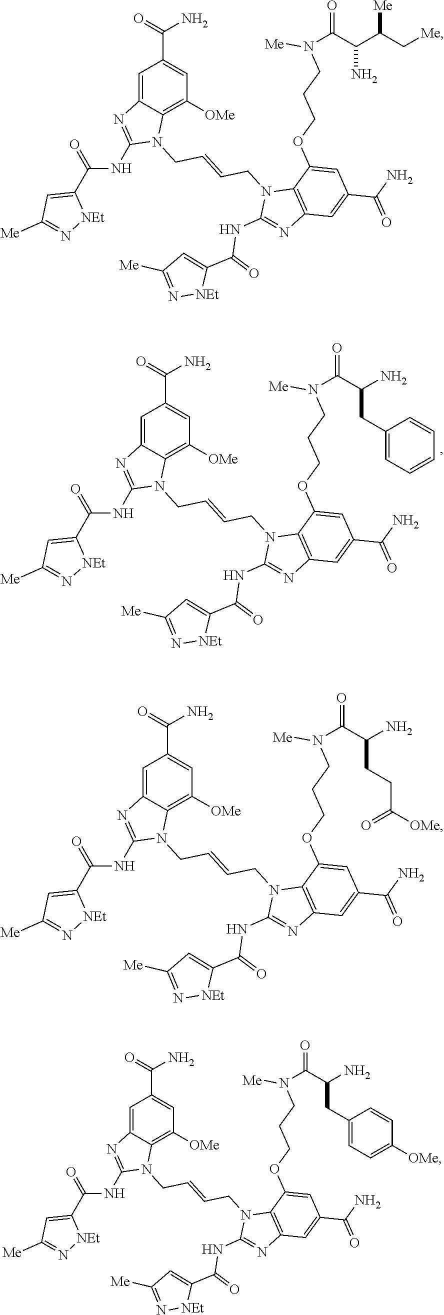

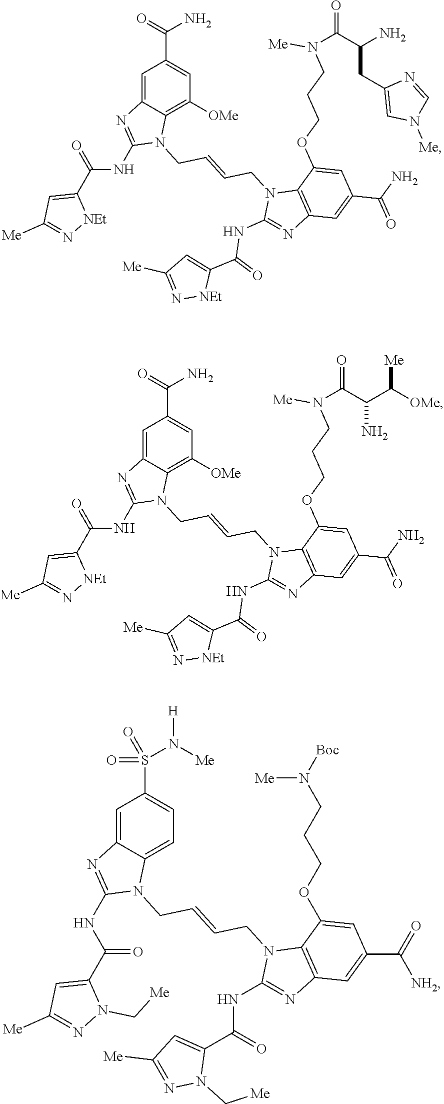

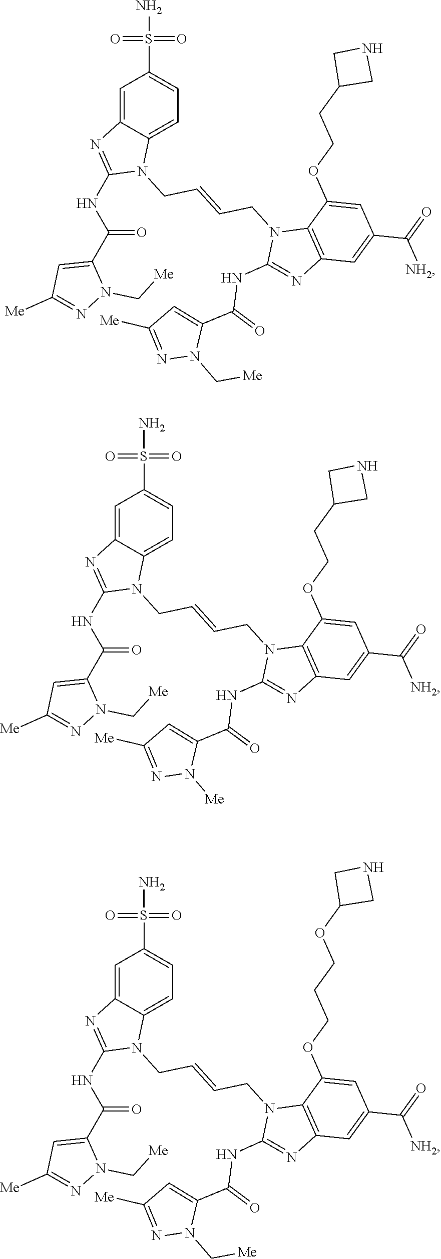 [SMILES: CCn1nc(C)cc1C(=O)Nc1nc2cc(S(N)(=O)=O)ccc2n1C/C=C/Cn1c(NC(=O)c2cc(C)nn2CC)nc2cc(C(N)=O)cc(OCCC3CNC3)c21 ; CCn1nc(C)cc1C(=O)Nc1nc2cc(S(N)(=O)=O)ccc2n1C/C=C/Cn1c(NC(=O)c2cc(C)nn2C)nc2cc(C(N)=O)cc(OCCC3CNC3)c21 ; CCn1nc(C)cc1C(=O)Nc1nc2cc(S(N)(=O)=O)ccc2n1C/C=C/Cn1c(NC(=O)c2cc(C)nn2CC)nc2cc(C(N)=O)cc(OCCCOC3CNC3)c21]

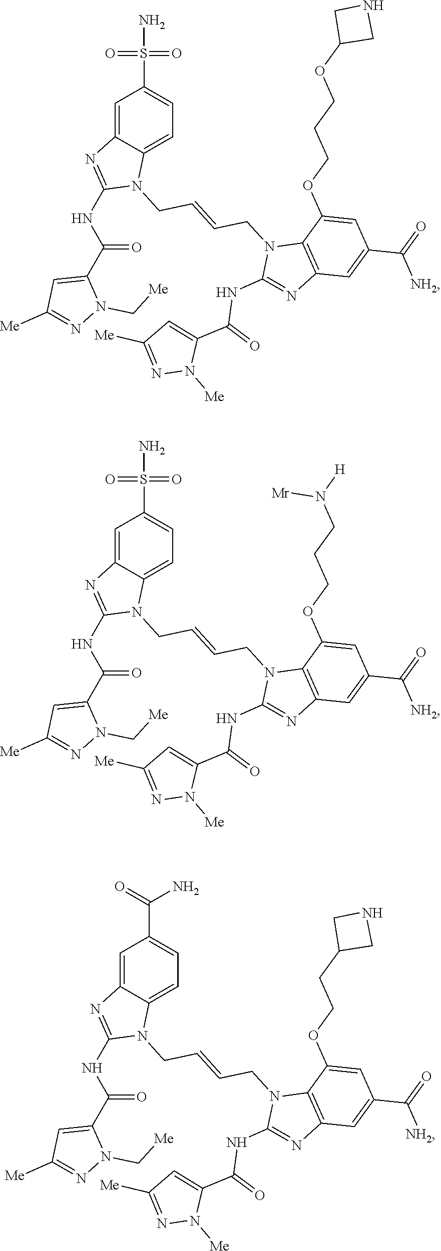

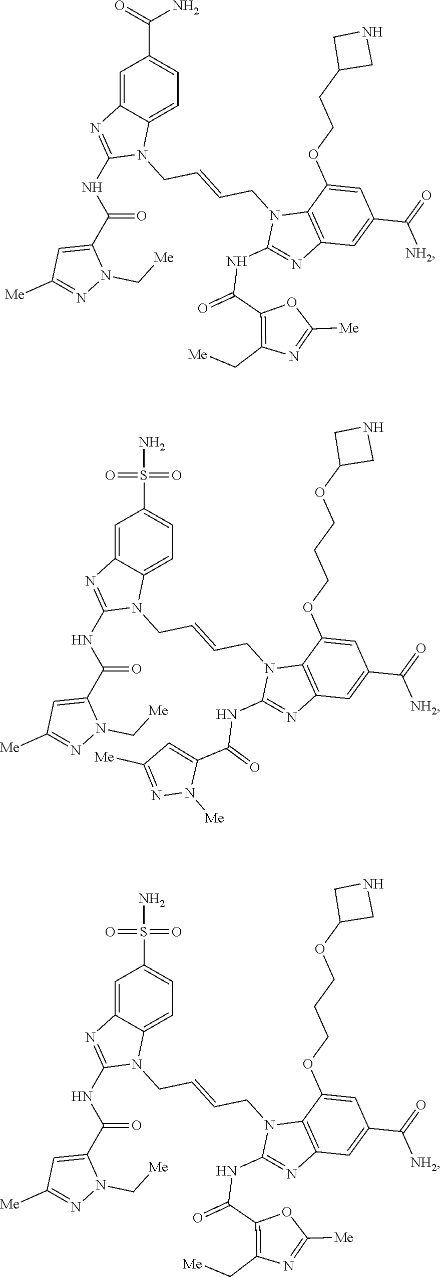 [SMILES: CCc1nc(C)oc1C(=O)Nc1nc2cc(C(N)=O)cc(OCCC3CNC3)c2n1C/C=C/Cn1c(NC(=O)c2cc(C)nn2CC)nc2cc(C(N)=O)ccc21 ; CCn1nc(C)cc1C(=O)Nc1nc2cc(S(N)(=O)=O)ccc2n1C/C=C/Cn1c(NC(=O)c2cc(C)nn2C)nc2cc(C(N)=O)cc(OCCCOC3CNC3)c21 ; CCc1nc(C)oc1C(=O)Nc1nc2cc(C(N)=O)cc(OCCCOC3CNC3)c2n1C/C=C/Cn1c(NC(=O)c2cc(C)nn2CC)nc2cc(S(N)(=O)=O)ccc21]

and pharmaceutically acceptable salts thereof.

### Linkers

As described herein, linkers (L) as defined in connection with Formulae (I), (II), and (II-A) are optional groups that connect X^{A} or X^{B}, when present, with M or M¹. For example, A, when present, is covalently attached to M or M¹, and Y, when present, is attached to X^{B} or to X^{A} (when X^{B} is absent). In some embodiments, the linker (L) has the formula -(A)ₐ-(W)_{w}-(Y)_{y}, wherein:
A is a C₂₋₂₀ alkylene optionally substituted with 1-3 R^{a1}; or a 2 to 40 membered heteroalkylene optionally substituted with 1-3 R^{b1};
each R^{a1} is independently selected from the group consisting of:
C₁₋₆ alkyl, C₁₋₆ haloalkyl, C₁₋₆ alkoxy, C₁₋₆ haloalkoxy, halogen, —OH, ═O, —NR^{d1}R^{e1}, —C(O)NR^{d1}R^{e1}, —C(O)(C₁₋₆ alkyl), and —C(O)O(C₁₋₆ alkyl);
each R^{b1} is independently selected from the group consisting of:
C₁₋₆ alkyl, C₁₋₆ haloalkyl, C₁₋₆ alkoxy, C₁₋₆ haloalkoxy, halogen, —OH, ═O, —NR^{d1}R^{e1}, —C(O)NR^{d1}R^{e1}, —C(O)(C₁₋₆ alkyl), and —C(O)O(C₁₋₆ alkyl);
each R^{d1} and R^{e1} are independently hydrogen or C₁₋₃ alkyl;
a is 00r 1;
W is from 1-12 amino acids or has the structure:

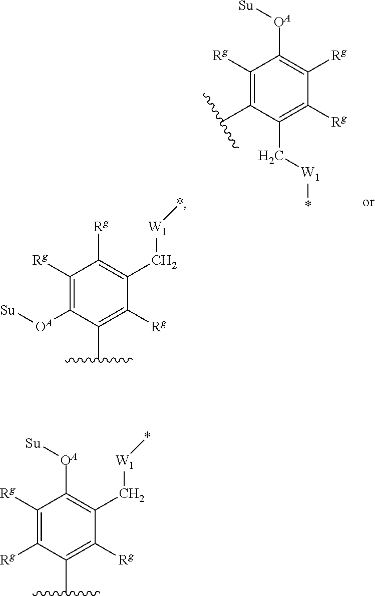

wherein Su is a Sugar moiety;
—O^{A}— represents a glycosidic bond;
each R^{g} is independently hydrogen, halogen, —CN, or —NO₂;
W₁ is absent or —O—C(═O)—;
[symbol] represents covalent attachment to A, when present, or M in compounds of Formula (II) and covalent attachment to A, M, or M¹ in the ADCs and compounds described herein;
* represents covalent attachment to Y, X^{A}, or X^{B} in compounds of Formula (II) and to Y, X^{A}, or X^{B} in the ADCs described herein;
w is 0 or 1;
Y is a self-immolative moiety, a non-self-immolative releasable moiety, or a non-cleavable moiety; and
y is 0 or 1.

In some embodiments, —O^{A}— represents a glycosidic bond. In some embodiments, the glycosidic bond provides a β-glucuronidase or a β-mannosidase-cleavage site. In some embodiments, the β-glucuronidase-cleavage site is cleavable by human lysosomal β-glucuronidase. In some embodiments, the β-mannosidase-cleavage site is cleavable by human lysosomal β-mannosidase.

In some embodiments, a is 0. In some embodiments, a is 1. In some embodiments, w is 0. In some embodiments, w is 1. In some embodiments, y is 0. In some embodiments, y is 1. In some embodiments, a+y+w=1. In some embodiments, a+y+w=2. In some embodiments, a+y+w=3. In some embodiments, a+y+w=0 (i.e., the linker (L) is absent).

In some embodiments, A is a C₂₋₂₀ alkylene optionally substituted with 1-3 R^{a1} In some embodiments, A is a C₂₋₁₀ alkylene optionally substituted with 1-3 R^{a1}. In some embodiments, A is a C₄₋₁₀ alkylene optionally substituted with 1-3 R^{a1}. In some embodiments, A is a C₂₋₂₀ alkylene substituted with R^{a1}. In some embodiments, A is a C₂₋₁₀ alkylene substituted with R^{a1}. In some embodiments, A is a C₂₋₁₀ alkylene substituted with R^{a1}.

In some embodiments, each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —C(O)NR^{d1}R^{e1}, —C(O)(C₁₋₆ alkyl), and —C(O)O(C₁₋₆ alkyl). In some embodiments, each R^{a1} is C₁₋₆ alkyl. In some embodiments, each R^{a1} is C₁₋₆ haloalkyl. In some embodiments, each R^{a1} is C₁₋₆ alkoxy. In some embodiments, each R^{a1} is C₁₋₆ haloalkoxy. In some embodiments, each R^{a1} is halogen. In some embodiments, each R^{a1} is —OH. In some embodiments, each R^{a1} is =0. In some embodiments, each R^{a1} is —NR^{d1}R^{e1}. In some embodiments, each R^{a1} is C(O)NR^{d1}R^{e1}. In some embodiments, each R^{a1} is —C(O)(C₁₋₆ alkyl). In some embodiments, each R^{a1} is —C(O)O(C₁₋₆ alkyl). In some embodiments, one occurrence of R^{a1} is —NR^{d1}R^{e1}. In some embodiments, A is a C₂₋₂₀ alkylene substituted with 1 or 2 R^{a1}, each of which is ═O.

In some embodiments, R^{d1} and R^{e1} are independently hydrogen or C₁₋₃ alkyl. In some embodiments, one of R^{d1} and R^{e1} is hydrogen, and the other of R^{d1} and R^{e1} is C₁₋₃ alkyl. In some embodiments, R^{d1} and R^{e1} are both hydrogen or C₁₋₃ alkyl. In some embodiments, R^{d1} and R^{e1} are both C₁₋₃ alkyl. In some embodiments, R^{d1} and R^{e1} are both methyl.

In some embodiments, A is a C₂₋₂₀ alkylene. In some embodiments, A is a C₂₋₁₀ alkylene. In some embodiments, A is a C₂₋₁₀ alkylene. In some embodiments, A is a C₂₋₆ alkylene. In some embodiments, A is a C₄₋₁₀ alkylene.

In some embodiments, A is a 2 to 40 membered heteroalkylene optionally substituted with 1-3 R^{b1}. In some embodiments, A is a 2 to 20 membered heteroalkylene optionally substituted with 1-3 R^{b1}. In some embodiments, A is a 2 to 12 membered heteroalkylene optionally substituted with 1-3 R^{b1}. In some embodiments, A is a 4 to 12 membered heteroalkylene optionally substituted with 1-3 R^{b1}. In some embodiments, A is a 4 to 8 membered heteroalkylene optionally substituted with 1-3 R^{b1}. In some embodiments, A is a 2 to 40 membered heteroalkylene substituted with R^{b1}. In some embodiments, A is a 2 to 20 membered heteroalkylene substituted with R^{b1}. In some embodiments, A is a 2 to 12 membered heteroalkylene substituted with R^{b1}. In some embodiments, A is a 4 to 12 membered heteroalkylene substituted with R^{b1}. In some embodiments, A is a 4 to 8 membered heteroalkylene substituted with R^{b1}.

In some embodiments, each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —C(O)NR^{d1}R^{e1}, —C(O)(C₁₋₆ alkyl), and —C(O)O(C₁₋₆ alkyl). In some embodiments, each R^{b1} is C₁₋₆ alkyl. In some embodiments, each R^{b1} is C₁₋₆ haloalkyl. In some embodiments, each R^{b1} is C₁₋₆ alkoxy. In some embodiments, each R^{b1} is C₁₋₆ haloalkoxy. In some embodiments, each R^{b1} is halogen. In some embodiments, each R^{b1} is —OH. In some embodiments, each R^{b1} is —NR^{d1}R^{e1}. In some embodiments, each R^{b1} is C(O)NR^{d1}R^{e1}. In some embodiments, each R^{b1} is —C(O)(C₁₋₆ alkyl). In some embodiments, each R^{b1} is —C(O)O(C₁₋₆ alkyl). In some embodiments, one occurrence of R^{b1} is —NR^{d1}R^{e1}.

In some embodiments, R^{d1} and R^{e1} are independently hydrogen or C₁₋₃ alkyl. In some embodiments, one of R^{d1} and R^{e1} is hydrogen, and the other of R^{d1} and R^{e1} is C₁₋₃ alkyl. In some embodiments, R^{d1} and R^{e1} are both hydrogen or C₁₋₃ alkyl. In some embodiments, R^{d1} and R^{e1} are both C₁₋₃ alkyl. In some embodiments, R^{d1} and R^{e1} are both methyl.

In some embodiments, A is a 2 to 40 membered heteroalkylene. In some embodiments, A is a 2 to 20 membered heteroalkylene. In some embodiments, A is a 2 to 12 membered heteroalkylene. In some embodiments, A is a 4 to 12 membered heteroalkylene. In some embodiments, A is a 4 to 8 membered heteroalkylene. In some embodiments, A is selected from the group consisting of:

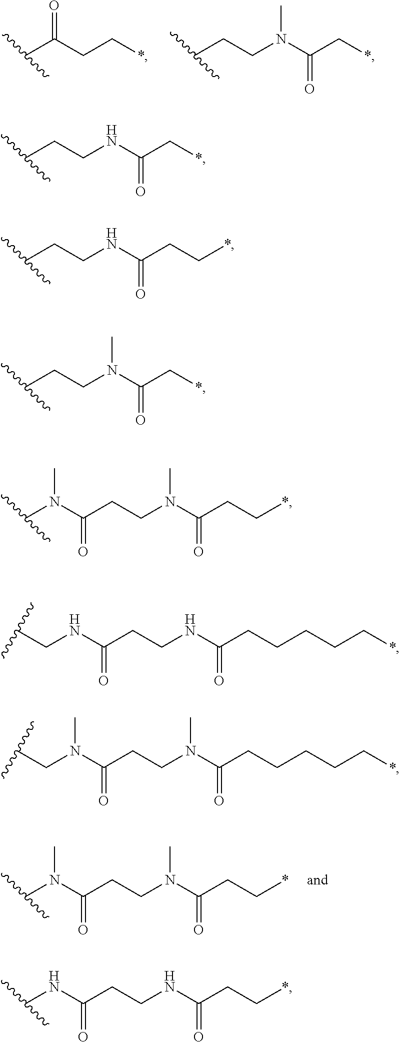

wherein [symbol] represents covalent attachment to W or Y, and * represents covalent linkage to M¹ or M (e.g., in compounds of Formula (I) or (II), respectively). In some embodiments, M is a succinimide. In some embodiments, M is a hydrolyzed succinimide. In some embodiments, M¹ is a succinimide. In some embodiments, M¹ is a hydrolyzed succinimide. It will be understood that a hydrolyzed succinimide may exist in two regioisomeric form(s). Those forms are exemplified below for hydrolysis of M, wherein the structures representing the regioisomers from that hydrolysis are formula M′ and M″; wherein the wavy lines adjacent to the bonds are as defined for A.

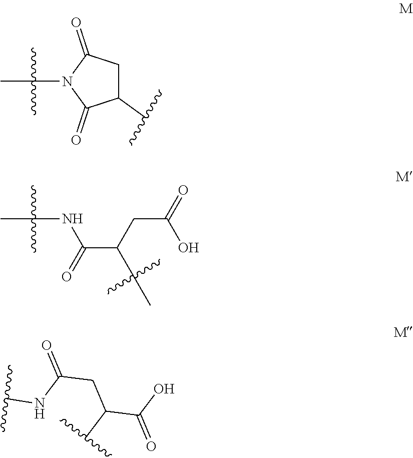 [SMILES: *C1CC(=O)N(*)C1=O ; *NC(=O)C(*)CC(=O)O ; *NC(=O)CC(*)C(=O)O]

In some embodiments, M′ is

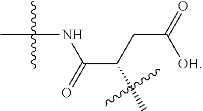 [SMILES: *NC(=O)[C@@H](C)CC(=O)O]

In some embodiments, M′ is

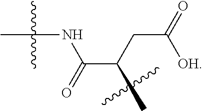 [SMILES: *NC(=O)[C@H](C)CC(=O)O]

In some embodiments, M″ is

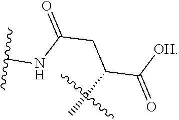 [SMILES: *NC(=O)C[C@@H](C)C(=O)O]

In some embodiments, M″ is

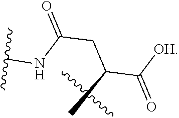 [SMILES: *NC(=O)C[C@H](C)C(=O)O]

In some embodiments, A is a PEG4 to PEG12. In some embodiments, A is a PEG4 to PEG8. Representative A groups include, but are not limited to:

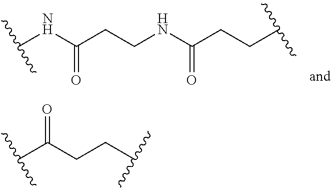

In some embodiments, w is 0. In some embodiments w is 1.

In some embodiments, W is a single amino acid. In some embodiments, W is a single natural amino acid. In some embodiments, W is a peptide including from 2-12 amino acids, wherein each amino acid is independently a natural or unnatural amino acid. In some embodiments, the natural or unnatural amino acid is a D or L isomer. In some embodiments, each amino acid is independently a natural amino acid. In some embodiments, each W is independently an alpha, beta, or gamma amino acid that is natural or unnatural. In some embodiments, W comprises a natural amino acid linked to an unnatural amino acid. In some embodiments, W comprises a natural or unnatural amino acid linked to a D-isomer of a natural or unnatural amino acid. In some embodiments, W is a dipeptide. In some embodiments, W is a tripeptide. In some embodiments, W is a tetrapeptide. In some embodiments, W is a pentapeptide. In some embodiments, W is a hexapeptide. In some embodiments, W is 7, 8, 9, 10, 11, or 12 amino acids. In some embodiments, each amino acid of W is independently selected from the group consisting of valine, alanine, β-alanine, glycine, lysine, leucine, phenylalanine, proline, aspartic acid, serine, glutamic acid, homoserine methyl ether, aspartate methyl ester, N,N-dimethyl lysine, arginine, valine-alanine, valine-citrulline, phenylalanine-lysine, and citrulline. In some embodiments, W is an aspartic acid. In some embodiments, W is a lysine. In some embodiments, W is a glycine. In some embodiments, W is an alanine. In some embodiments, W is aspartate methyl ester. In some embodiments, W is a N,N-dimethyl lysine. In some embodiments, W is a homoserine methyl ether. In some embodiments, W is a serine. In some embodiments, W is a valine-alanine.

In some embodiments, w is 1; W is from 1-12 amino acids; and the bond between W and the X^{B} or between W and Y is enzymatically cleavable by a tumor-associated protease. In some embodiments, the tumor-associated protease is a cathepsin. In some embodiments, the tumor-associated protease is cathepsin B, C, or D.

In some embodiments, w is 1; and W has the structure of:

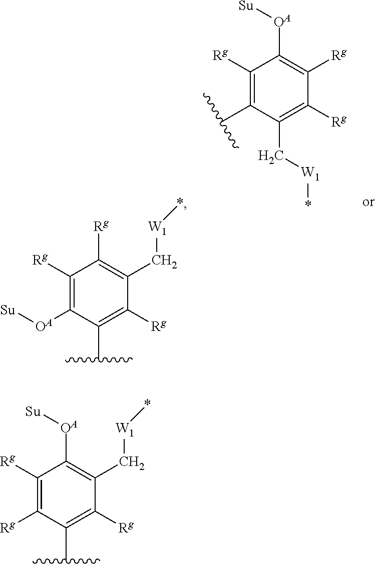

wherein Su is a Sugar moiety;
—O^{A}— represents a glycosidic bond;
each R^{g} is independently hydrogen, halogen, —CN, or —NO₂;
W₁ is absent or —O—C(═O)—;
[symbol] represents covalent attachment to A or M in compounds of Formula (II); and
the * represents covalent attachment to Y, X^{A}, or X^{B} in compounds of Formula (II);

In some embodiments, w is 1; and W has the structure of:

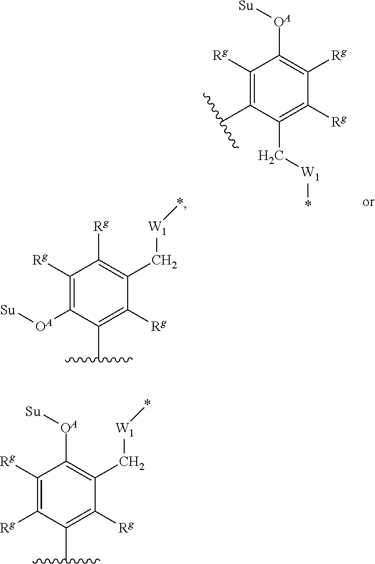

wherein Su is a Sugar moiety;
—O^{A}— represents a glycosidic bond;
each R^{g} is independently hydrogen, halogen, —CN, or —NO₂;
W₁ is absent or —O—C(═O)—;
[symbol] represents covalent attachment to A or M in the ADCs described herein; and
the * represents covalent attachment to Y, X^{A}, or X^{B} in the ADCs described herein;

In some embodiments, —O^{A}— represents a glycosidic bond. In some embodiments, the glycosidic bond provides a β-glucuronidase or a β-mannosidase-cleavage site. In some embodiments, the β-glucuronidase or a β-mannosidase-cleavage site is cleavable by human lysosomal β-glucuronidase or by human lysosomal β-mannosidase.

In some embodiments, W is

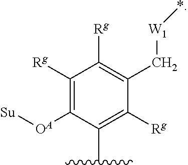

In some embodiments, W is

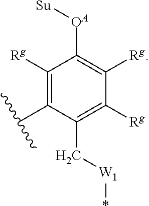

In some embodiments, W is

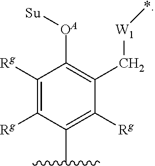

In some embodiments, each R^{g} is hydrogen. In some embodiments, one R^{g} is hydrogen, and the remaining R^{g} are independently halo, —CN, or —NO₂. In some embodiments, two R^{g} are hydrogen, and the remaining R^{g} is halo, —CN, or —NO₂.

In some embodiments, one R^{g} is halogen, —CN, or —NO₂, and the other R^{g} are hydrogen. In some embodiments, each R^{g} is hydrogen.

In some embodiments, O^{A}-Su is charged neutral at physiological pH. In some embodiments, O^{A}-Su is mannose. In some embodiments, O^{A}-Su is

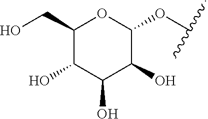

In some embodiments, O^{A}-Su comprises a carboxylate moiety. In some embodiments, O^{A}-Su is glucuronic acid. In some embodiments, O^{A}-Su is

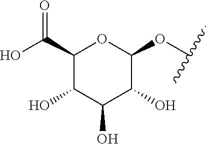

In some embodiments, W is

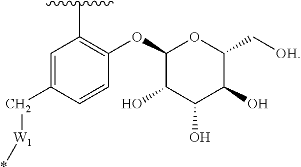

In some embodiments, W is

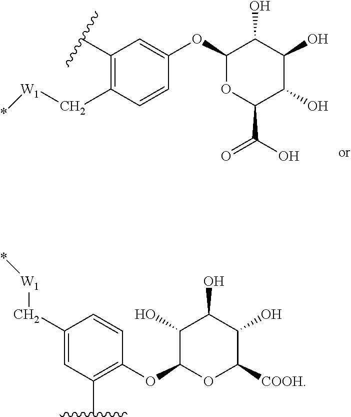

In some embodiments, W is

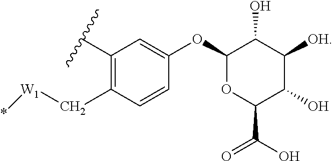

In some, embodiments, W is

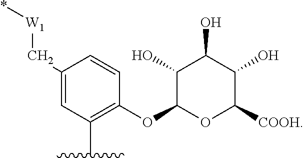

In some embodiments, a is 0.

In some embodiments, y is 0. In some embodiments y is 1.

In some embodiments, Y is a self-immolative moiety, a non-self-immolative releasable moiety, or a non-cleavable moiety. In some embodiments, Y is a self-immolative moiety or a non-self-immolative releasable moiety. In some embodiments, Y is a self-immolative moiety. In some embodiments, Y is a non-self-immolative moiety.

A non-self-immolative moiety is one which requires enzymatic cleavage, and in which part or all of the group remains bound to the Drug Unit after cleavage from the ADC, thereby forming free drug. Examples of a non-self-immolative moiety include, but are not limited to: -glycine-; and -glycine-glycine. When an ADC having Y is -glycine- or -glycine-glycine-undergoes enzymatic cleavage (for example, via a cancer-cell-associated protease or a lymphocyte-associated protease), the Drug Unit is cleaved from the ADC such that the free drug includes the glycine or glycine-glycine group from Y. In some embodiments, an independent hydrolysis reaction takes place within, or in proximity to, the target cell, further cleaving the glycine or glycine-glycine group from the free drug. In some embodiments, enzymatic cleavage of the non-self-immolative moiety, as described herein, does not result in any further hydrolysis step(s).

A self-immolative moiety refers to a bifunctional chemical moiety that is capable of covalently linking together two spaced chemical moieties into a normally stable tripartite molecule. The self-immolative group will spontaneously separate from the second chemical moiety if its bond to the first moiety is cleaved. For example, a self-immolative moiety includes a β-aminobenzyl alcohol (PAB) optionally substituted with one or more alkyl, alkoxy, halogen, cyano, or nitro groups. Other examples of self-immolative moieties include, but are not limited to, aromatic compounds that are electronically similar to the PAB group such as 2-aminoimidazol-5-methanol derivatives (see, e.g., Hay et al., 1999, Bioorg. Med. Chem. Lett. 9:2237), ortho or para-aminobenzylacetals, substituted and unsubstituted 4-aminobutyric acid amides (see, e.g., Rodrigues et al., 1995, Chemistry Biology 2:223), appropriately substituted bicyclo[2.2.1] and bicyclo[2.2.2] ring systems (see, e.g., Storm et al., 1972, J. Amer. Chem. Soc. 94:5815), 2-aminophenylpropionic acid amides (see, e.g., Amsberry et al., 1990, J. Org. Chem. 55:5867), and elimination of amine-containing drugs that are substituted at the α-position of glycine (see, e.g., Kingsbury et al., 1984, J. Med. Chem. 27:1447).

In some embodiments, Y is a PAB group, optionally substituted with one or more alkyl, alkoxy, halogen, cyano, or nitro groups; a para-aminobenzyloxy-carbonyl (PABC) group optionally substituted with a sugar moiety; -glycine-; -glycine-glycine-; or a branched bis(hydroxymethyl)styrene (BHMS) unit, which is capable of incorporating (and releasing) multiple Drug Units.

In some embodiments, -(A)ₐ-(W)_{w}-(Y)_{y} comprises a non-self-immolative releasable linker, which provides release of the free drug once the ADC has been internalized into the target cell. In some embodiments, -(A)ₐ-(W)_{w}-(Y)_{y} comprises a releasable linker, which provides release of the free Drug with, or in the vicinity, of targeted cells. Releasable linkers possess a recognition site, such as a peptide cleavage site, sugar cleavage site, or disulfide cleavage side. In some embodiments, each releasable linker is a di-peptide. In some embodiments, each releasable linker is a disulfide. In some embodiments, each releasable linker is a hydrazone. In some embodiments, each releasable linker is independently Val-Cit-, -Phe-Lys-, or -Val-Ala-. In some embodiments, each releasable linker, when bound to a succinimide or hydrolyzed succinimide, is independently succinimido-caproyl (mc), succinimido-caproyl-valine-citrulline (sc-vc), succinimido-caproyl-valine-citrulline-paraaminobenzyloxycarbonyl (sc-vc-PABC), or SDPr-vc (where “S” refers to succinimido).

In some embodiments, -(A)ₐ-(W)_{w}-(Y)_{y} comprises a non-cleavable linker. Non-cleavable linkers are known in the art and can be adapted for use with the ADCs described herein as the “Y” group. A non-cleavable linker is capable of linking a Drug Unit to an antibody in a generally stable and covalent manner and is substantially resistant to acid-induced cleavage, light-induced cleavage, peptidase- or esterase-induced cleavage, and disulfide bond cleavage. The free drug can be released from the ADCs containing non-cleavable linkers via alternative mechanisms, such as proteolytic antibody degradation. In some embodiments, the Drug Unit can exert a biological effect as a part of the ADC (i.e., while still conjugated to the antibody via a linker).

Reagents that form non-cleavable linker-maleimide and non-cleavable linker-succinimide compounds are known in the art and can adapted for use herein. Exemplary reagents comprise a maleimido or haloacetyl-based moiety, such as 6-maleimidocaproic acid N-hydroxy succinimide ester (MCC), N-succinimidyl 4-(maleimidomethyl)cyclohexanecarboxylate (SMCC), N-succinimidyl-4-(N-maleimidomethyl)-cyclohexane-1-carboxy-(6-amidocaproate) (LC-SMCC), maleimidoundecanoic acid N-succinimidyl ester (KMUA), γ-maleimidobutyric acid N-succinimidyl ester (GMBS), c-maleimidocaproic acid N-hydroxysuccinimide ester (EMCS), m-maleimidobenzoyl-N-hydroxysuccinimide ester (MBS), N-(α-maleimidoacetoxy)-succinimide ester [AMAS], succinimidyl-6-(β-maleimidopropionamido)hexanoate (SMPH), N-succinimidyl 4-(β-maleimidophenyl)-butyrate (SMPB), and N-(β-maleimidophenyl)isocyanate (PMPI), N-succinimidyl-4-(iodoacetyl)-aminobenzoate (STAB), N-succinimidyl iodoacetate (SIA), N-succinimidyl bromoacetate (SBA) and N-succinimidyl 3-(bromoacetamido)propionate (SBAP). Additional “A-M” and “A-M¹” groups for use in the ADCs described herein can be found, for example, in U.S. Pat. No. 8,142,784, incorporated herein by reference in its entirety.

In some embodiments, y is 1; and Y is

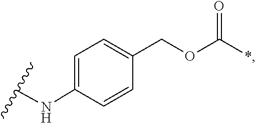

wherein [symbol] represents connection to W, A, or M in compounds of Formula (II); and the * represents connection to X^{A} or X^{B}, in compounds of Formula (II).

In some embodiments, y is 1; and Y is

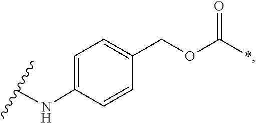

wherein [symbol] represents connection to W, A, M or M¹ in the ADCs described herein; and the * represents connection to X^{A} or X^{B}, in the ADCs described herein.

In some embodiments, -(A)ₐ-(W)_{w}-(Y)_{y}- comprises a non-releasable linker, wherein the Drug is released after the ADC has been internalized into the target cell and degraded, liberating the Drug.

In some embodiments, the linker (L) is substituted with a polyethylene glycol moiety selected from the group consisting of PEG2 to PEG20. In some embodiments, L is substituted with a polyethylene glycol moiety selected from the group consisting of PEG2, PEG4, PEG6, PEG8, PEG10, PEG12, PEG16, and PEG20. In some embodiments, L is not substituted with a polyethylene glycol moiety selected from the group consisting of PEG2 to PEG20.

Polydisperse PEGs, monodisperse PEGs and discrete PEGs can be used to make the ADCs and intermediates thereof. Polydisperse PEGs are a heterogeneous mixture of sizes and molecular weights whereas monodisperse PEGs are typically purified from heterogeneous mixtures and therefore provide a single chain length and molecular weight. Discrete PEGs are synthesized in step-wise fashion and not via a polymerization process. Discrete PEGs provide a single molecule with defined and specified chain length. The number of —CH₂CH₂O— subunits of a PEG Unit ranges, for example, from 8 to 24 or from 12 to 24, referred to as PEG8 to PEG24 and PEG12 to PEG24, respectively.

The PEG moieties provided herein, which are also referred to as PEG Units, comprise one or multiple polyethylene glycol chains. The polyethylene glycol chains are linked together, for example, in a linear, branched or star shaped configuration. Typically, at least one of the polyethylene glycol chains of a PEG Unit is derivatized at one end for covalent attachment to an appropriate site on a component of the ADC (e.g., L). Exemplary attachments to ADCs are by means of non-conditionally cleavable linkages or via conditionally cleavable linkages. Exemplary attachments are via amide linkage, ether linkages, ester linkages, hydrazone linkages, oxime linkages, disulfide linkages, peptide linkages or triazole linkages. In some embodiments, attachment to the Formula (I) ADC is by means of a non-conditionally cleavable linkage. In some embodiments, attachment to the ADC is not via an ester linkage, hydrazone linkage, oxime linkage, or disulfide linkage. In some embodiments, attachment to the ADC is not via a hydrazone linkage.

A conditionally cleavable linkage refers to a linkage that is not substantially sensitive to cleavage while circulating in plasma but is sensitive to cleavage in an intracellular or intratumoral environment. A non-conditionally cleavable linkage is one that is not substantially sensitive to cleavage in any biologically relevant environment in a subject that is administered the ADC. Chemical hydrolysis of a hydrazone, reduction of a disulfide bond, and enzymatic cleavage of a peptide bond or glycosidic bond of a Glucuronide Unit as described by WO 2007/011968 (which is incorporated by reference in its entirety) are examples of conditionally cleavable linkages.

In some embodiments, the PEG Unit is directly attached to the ADC (or an intermediate thereof) at L. In those embodiments, the other terminus (or termini) of the PEG Unit is free and untethered (i.e., not covalently attached), and in some embodiments, is a methoxy, carboxylic acid, alcohol or other suitable functional group. The methoxy, carboxylic acid, alcohol or other suitable functional group acts as a cap for the terminal polyethylene glycol subunit of the PEG Unit. By untethered, it is meant that the PEG Unit will not be covalently attached at that untethered site to a Drug Unit, to an antibody, or to a linking component to a Drug Unit and/or an antibody. Such an arrangement can allow a PEG Unit of sufficient length to assume a parallel orientation with respect to the drug in conjugated form, i.e., as a Drug Unit (D). For those embodiments in which the PEG Unit comprises more than one polyethylene glycol chain, the multiple polyethylene glycol chains are independently chosen, e.g., are the same or different chemical moieties (e.g., polyethylene glycol chains of different molecular weight or number of —CH₂CH₂O— subunits). A PEG Unit having multiple polyethylene glycol chains is attached to the ADC at a single attachment site. The skilled artisan will understand that the PEG Unit, in addition to comprising repeating polyethylene glycol subunits, may also contain non-PEG material (e.g., to facilitate coupling of multiple polyethylene glycol chains to each other or to facilitate coupling to the ADC). Non-PEG material refers to the atoms in the PEG Unit that are not part of the repeating —CH₂CH₂O— subunits. In some embodiments provided herein, the PEG Unit comprises two monomeric polyethylene glycol chains attached to each other via non-PEG elements. In other embodiments provided herein, the PEG Unit comprises two linear polyethylene glycol chains attached to a central core that is attached to the ADC (i.e., the PEG Unit itself is branched).

There are a number of PEG attachment methods available to those skilled in the art: see, for example: Goodson, et al. (1990) Bio/Technology 8:343 (PEGylation of interleukin-2 at its glycosylation site after site-directed mutagenesis); EP 0 401 384 (coupling PEG to G-CSF); Malik, et al., (1992) Exp. Hematol. 20:1028-1035 (PEGylation of GM-CSF using tresyl chloride); ACT Pub. No. WO 90/12874 (PEGylation of erythropoietin containing a recombinantly introduced cysteine residue using a cysteine-specific mPEG derivative); U.S. Pat. No. 5,757,078 (PEGylation of EPO peptides); U.S. Pat. No. 5,672,662 (Poly(ethylene glycol) and related polymers monosubstituted with propionic or butanoic acids and functional derivatives thereof for biotechnical applications); U.S. Pat. No. 6,077,939 (PEGylation of an N-terminal α-carbon of a peptide); Veronese et al., (1985) Appl. Biochem. Bioechnol 11:141-142 (PEGylation of an N-terminal α-carbon of a peptide with PEG-nitrophenylcarbonate (“PEG-NPC”) or PEG-trichlorophenylcarbonate); and Veronese (2001) Biomaterials 22:405-417 (Review article on peptide and protein PEGylation).

For example, a PEG Unit may be covalently bound to an amino acid residue via reactive groups of a polyethylene glycol-containing compound and the amino acid residue. Reactive groups of the amino acid residue include those that are reactive to an activated PEG molecule (e.g., a free amino or carboxyl group). For example, N-terminal amino acid residues and lysine (K) residues have a free amino group; and C-terminal amino acid residues have a free carboxyl group. Thiol groups (e.g., as found on cysteine residues) are also useful as a reactive group for forming a covalent attachment to a PEG. In addition, enzyme-assisted methods for introducing activated groups (e.g., hydrazide, aldehyde, and aromatic-amino groups) specifically at the C-terminus of a polypeptide have been described. See Schwarz, et al. (1990) Methods Enzymol. 184:160; Rose, et al. (1991) Bioconjugate Chem. 2:154; and Gaertner, et al. (1994) J. Biol. Chem. 269: 7224.

In some embodiments, a polyethylene glycol-containing compound forms a covalent attachment to an amino group using methoxylated PEG (“mPEG”) having different reactive moieties. Non-limiting examples of such reactive moieties include succinimidyl succinate (SS), succinimidyl carbonate (SC), mPEG-imidate, para-nitrophenylcarbonate (NPC), succinimidyl propionate (SPA), and cyanuric chloride. Non-limiting examples of such mPEGs include mPEG-succinimidyl succinate (mPEG-SS), mPEG₂-succinimidyl succinate (mPEG₂-SS); mPEG-succinimidyl carbonate (mPEG-SC), mPEG₂-succinimidyl carbonate (mPEG₂-SC); mPEG-imidate, mPEG-para-nitrophenylcarbonate (mPEG-NPC), mPEG-imidate; mPEG₂-para-nitrophenylcarbonate (mPEG₂-NPC); mPEG-succinimidyl propionate (mPEG-SPA); mPEG₂-succinimidyl propionate (mPEG-SPA); mPEG-N-hydroxy-succinimide (mPEG-NHS); mPEG₂-N-hydroxy-succinimide (mPEG₂-NHS); mPEG-cyanuric chloride; mPEG₂-cyanuric chloride; mPEG₂-Lysinol-NPC, and mPEG₂-Lys-NHS.

Generally, at least one of the polyethylene glycol chains that make up the PEG is functionalized to provide covalent attachment to the ADC. Functionalization of the polyethylene glycol-containing compound that is the precursor to the PEG includes, for example, via an amine, thiol, NHS ester, maleimide, alkyne, azide, carbonyl, or other functional group. In some embodiments, the PEG further comprises non-PEG material (i.e., material not comprised of —CH₂CH₂O—) that provides coupling to the ADC or in constructing the polyethylene glycol-containing compound or PEG facilitates coupling of two or more polyethylene glycol chains.

In some embodiments, the presence of the PEG Unit in an ADC is capable of having two potential impacts upon the pharmacokinetics of the resulting ADC. One impact is a decrease in clearance (and consequent increase in exposure) that arises from the reduction in non-specific interactions induced by the exposed hydrophobic elements of the Drug Unit. The second impact is a decrease in volume and rate of distribution that sometimes arises from the increase in the molecular weight of the ADC. Increasing the number of polyethylene glycol subunits increases the hydrodynamic radius of a conjugate, typically resulting in decreased diffusivity. In turn, decreased diffusivity typically diminishes the ability of the ADC to penetrate into a tumor. See Schmidt and Wittrup, Mol Cancer Ther 2009; 8:2861-2871. Because of these two competing pharmacokinetic effects, it can be desirable to use a PEG Unit that is sufficiently large to decrease the ADC clearance thus increasing plasma exposure, but not so large as to greatly diminish its diffusivity to an extent that it interferes with the ability of the ADC to reach the intended target cell population. See, e.g., Examples 1, 18, and 21 of US 2016/0310612, which is incorporated by reference herein (e.g., for methodology for selecting an optimal size of a PEG Unit for a particular Drug Unit, Linker, and/or drug-linker compound).

In one group of embodiments, the PEG Unit comprises one or more linear polyethylene glycol chains each having at 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In some embodiments, the PEG comprises a combined total of at least 8 subunits, at least 10 subunits, or at least 12 subunits. In some such embodiments, the PEG comprises no more than a combined total of about 72 subunits. In some such embodiments, the PEG comprises no more than a combined total of about 36 subunits. In some embodiments, the PEG comprises about 8 to about 24 subunits (referred to as PEG8 to PEG24).

In another group of embodiments, the PEG Unit comprises a combined total of from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits.

Illustrative linear PEGs that can be used in any of the embodiments provided herein are as follows:

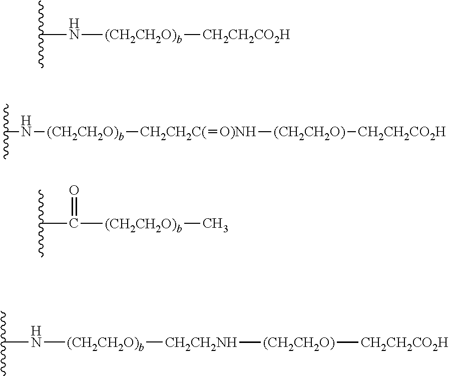

wherein the wavy line indicates the site of attachment to the ADC, and each subscript b is independently selected from the group consisting of 7 to 72, 8 to 72, 10 to 72, 12 to 72, 6 to 24, or 8 to 24. In some embodiments, each subscript b is about 8, about 12, or about 24.

As described herein, the PEG Unit can be selected such that it improves clearance of the resultant ADC but does not significantly impact the ability of the ADC to penetrate into the tumor.

In some embodiments, the PEG is from about 300 daltons to about 5 kilodaltons; from about 300 daltons to about 4 kilodaltons; from about 300 daltons to about 3 kilodaltons; from about 300 daltons to about 2 kilodaltons; from about 300 daltons to about 1 kilodalton; or any value in between. In some embodiments, the PEG has at least 8, 10 or 12 subunits. In some embodiments, the PEG Unit is PEG8 to PEG72, for example, PEG8, PEG10, PEG12, PEG16, PEG20, PEG24, PEG28, PEG32, PEG36, PEG48, or PEG72.

In some embodiments, apart from the PEGylation of the ADC, there are no other PEG subunits present in the ADC (i.e., no PEG subunits are present as part of any of the other components of the conjugates and linkers provided herein, such as A and X^{B}). In some embodiments, apart from the PEG, there are no more than 8, no more than 7, no more than 6, no more than 5, no more than 4, no more than 3, no more than 2 or no more than 1 other polyethylene glycol (—CH₂CH₂O—) subunits present in the ADC, or intermediate thereof (i.e., no more than 8, 7, 6, 5, 4, 3, 2, or 1 other polyethylene glycol subunits in other components of the ADCs (or intermediates thereof) provided herein).

It will be appreciated that when referring to polyethylene glycol subunits of a PEG Unit, and depending on context, the number of subunits can represent an average number, e.g., when referring to a population of ADCs or intermediates thereto and/or using polydisperse PEGs.

### Methods of Use

In some embodiments, the ADCs described herein, or pharmaceutically acceptable salts thereof, are used to deliver the conjugated drug to a target cell. Without being bound by theory, in some embodiments, an ADC associates with an antigen on the surface of a target cell. The Drug Unit can then be released as free drug to induce its biological effect (such as an immunostimulatory effect). The Drug Unit can also remain attached to the antibody, or a portion of the antibody and/or linker, and induce its biological effect.

Some embodiments provide a method of treating cancer in a subject in need thereof, comprising administering a therapeutically effective amount of an ADC described herein, or a pharmaceutically acceptable salt thereof, to the subject.

Some embodiments provide a method of treating cancer in a subject in need thereof, comprising administering a therapeutically effective amount of a composition comprising an ADC described herein, or a pharmaceutically acceptable salt thereof, to the subject.

Some embodiments provide a method of inducing an anti-tumor immune response in a subject in need thereof, comprising administering a therapeutically effective amount of a composition comprising a ADC described herein, or a pharmaceutically acceptable salt thereof, to the subject.

Some embodiments provide a method of inducing an anti-tumor immune response in a subject in need thereof, comprising administering a therapeutically effective amount of an ADC described herein, or a pharmaceutically acceptable salt thereof, to the subject.

Some embodiments provide a method of treating cancer in a subject in need thereof, comprising administering a therapeutically effective amount of an ADC as described herein, or a pharmaceutically acceptable salt thereof, to the subject in combination with another anticancer therapy (e.g., surgery and radiation therapy) and/or anticancer agent (e.g., an immunotherapy such as nivolumab or pembrolizumab). The ADCs described herein can be administered before, during, or after administration of the anticancer therapy and/or anticancer agent to the subject. In some embodiments, the ADCs described herein can be administered to the subject following treatment with radiation and/or after surgery.

Some embodiments provide a method for delaying or preventing acquired resistance to an anticancer agent, comprising administering a therapeutically effective amount of an ADC as described herein, or a pharmaceutically acceptable salt thereof, to a patient at risk for developing or having acquired resistance to an anticancer agent. In some embodiments, the patient is administered a dose of the anticancer agent (e.g., at substantially the same time as a dose of an ADC as described herein, or a pharmaceutically acceptable salt thereof is administered to the patient).

Some embodiments provide a method of delaying and/or preventing development of cancer resistant to an anticancer agent in a subject, comprising administering to the subject a therapeutically effective amount of an ADC as described herein, or a pharmaceutically acceptable salt thereof, before, during, or after administration of a therapeutically effective amount of the anticancer agent.

The ADCs described herein are useful for inhibiting the multiplication of a cancer cell, causing apoptosis in a cancer cell, for increasing phagocytosis of a cancer cell, and/or for treating cancer in a subject in need thereof. The ADCs can be used accordingly in a variety of settings for the treatment of cancers. The ADCs can be used to deliver a drug to a cancer cell. Without being bound by theory, in some embodiments, the antibody of an ADC binds to or associates with a cancer-cell-associated antigen. The antigen can be attached to a cancer cell or can be an extracellular matrix protein associated with the cancer cell. The drug can be released in proximity to the cancer cell, thus recruiting/activating immune cells to attack the cancer cell. In some embodiments, the Drug Unit is cleaved from the ADC outside the cancer cell. In some embodiments, the Drug Unit remains attached to the antibody bound to the antigen.

In some embodiments, the antibody binds to the cancer cell. In some embodiments, the antibody binds to a cancer cell antigen which is on the surface of the cancer cell. In some embodiments, the antibody binds to a cancer cell antigen which is an extracellular matrix protein associated with the tumor cell or cancer cell. In some embodiments, the antibody of an ADC binds to or associates with a cancer-associated cell or an antigen on a cancer-associated cell. In some embodiments, the cancer-associated cell is a stromal cell in a tumor, for example, a cancer-associated fibroblast (CAF).

In some embodiments, the antibody of an ADC binds to or associates with an immune cell or an immune-cell-associated antigen. The antigen can be attached to an immune cell or can be an extracellular matrix protein associated with the immune cell. The drug can be released in proximity to the immune cell, thus recruiting/activating the immune cell to attack a cancer cell. In some embodiments, the Drug Unit is cleaved from the ADC outside the immune cell. In some embodiments, the Drug Unit remains attached to the antibody bound to the antigen. In some embodiments, the immune cell is a lymphocyte, an antigen-presenting cell, a natural killer (NK) cell, a neutrophil, an eosinophil, a basophil, a mast cell, innate lymphoid cells or a combination of any of the foregoing. In some embodiments, the immune cell is selected from the group consisting of B cells, plasma cells, T cells, NKT cells, gamma delta T (γδT) cells, monocytes, macrophages, dendritic cells, natural killer (NK) cells, neutrophils, eosinophils, basophils, mast cells, innate lymphoid cells and a combination of any of the foregoing.

The specificity of the antibody for a particular cancer cell can be important for determining those tumors or cancers that are most effectively treated. For example, ADCs that target a cancer cell antigen present on hematopoietic cancer cells in some embodiments treat hematologic malignancies. In some embodiments, ADCs target a cancer cell antigen present on abnormal cells of solid tumors for treating such solid tumors. In some embodiments an ADC are directed against abnormal cells of hematopoietic cancers such as, for example, lymphomas (Hodgkin Lymphoma and Non-Hodgkin Lymphomas) and leukemias.

Cancers, including, but not limited to, a tumor, metastasis, or other disease or disorder characterized by abnormal cells that are characterized by uncontrolled cell growth in some embodiments are treated or inhibited by administration of an ADC.

In some embodiments, the subject has previously undergone treatment for the cancer. In some embodiments, the prior treatment is surgery, radiation therapy, administration of one or more anticancer agents, or a combination of any of the foregoing.

In any of the methods described herein, the cancer is selected from the group consisting of: adenocarcinoma, adrenal gland cortical carcinoma, adrenal gland neuroblastoma, anus squamous cell carcinoma, appendix adenocarcinoma, bladder urothelial carcinoma, bile duct adenocarcinoma, bladder carcinoma, bladder urothelial carcinoma, bone chordoma, bone marrow leukemia lymphocytic chronic, bone marrow leukemia non-lymphocytic acute myelocytic, bone marrow lymph proliferative disease, bone marrow multiple myeloma, bone sarcoma, brain astrocytoma, brain glioblastoma, brain medulloblastoma, brain meningioma, brain oligodendroglioma, breast adenoid cystic carcinoma, breast carcinoma, breast ductal carcinoma in situ, breast invasive ductal carcinoma, breast invasive lobular carcinoma, breast metaplastic carcinoma, cervix neuroendocrine carcinoma, cervix squamous cell carcinoma, colon adenocarcinoma, colon carcinoid tumor, duodenum adenocarcinoma, endometrioid tumor, esophagus adenocarcinoma, esophagus and stomach carcinoma, eye intraocular melanoma, eye intraocular squamous cell carcinoma, eye lacrimal duct carcinoma, fallopian tube serous carcinoma, gallbladder adenocarcinoma, gallbladder glomus tumor, gastroesophageal junction adenocarcinoma, head and neck adenoid cystic carcinoma, head and neck carcinoma, head and neck neuroblastoma, head and neck squamous cell carcinoma, kidney chromophore carcinoma, kidney medullary carcinoma, kidney renal cell carcinoma, kidney renal papillary carcinoma, kidney sarcomatoid carcinoma, kidney urothelial carcinoma, kidney carcinoma, leukemia lymphocytic, leukemia lymphocytic chronic, liver cholangiocarcinoma, liver hepatocellular carcinoma, liver carcinoma, lung adenocarcinoma, lung adenosquamous carcinoma, lung atypical carcinoid, lung carcinosarcoma, lung large cell neuroendocrine carcinoma, lung non-small cell lung carcinoma, lung sarcoma, lung sarcomatoid carcinoma, lung small cell carcinoma, lung small cell undifferentiated carcinoma, lung squamous cell carcinoma, upper aerodigestive tract squamous cell carcinoma, upper aerodigestive tract carcinoma, lymph node lymphoma diffuse large B cell, lymph node lymphoma follicular lymphoma, lymph node lymphoma mediastinal B-cell, lymph node lymphoma plasmablastic lung adenocarcinoma, lymphoma follicular lymphoma, lymphoma, non-Hodgkins, nasopharynx and paranasal sinuses undifferentiated carcinoma, ovary carcinoma, ovary carcinosarcoma, ovary clear cell carcinoma, ovary epithelial carcinoma, ovary granulosa cell tumor, ovary serous carcinoma, pancreas carcinoma, pancreas ductal adenocarcinoma, pancreas neuroendocrine carcinoma, peritoneum mesothelioma, peritoneum serous carcinoma, placenta choriocarcinoma, pleura mesothelioma, prostate acinar adenocarcinoma, prostate carcinoma, rectum adenocarcinoma, rectum squamous cell carcinoma, skin adnexal carcinoma, skin basal cell carcinoma, skin melanoma, skin Merkel cell carcinoma, skin squamous cell carcinoma, small intestine adenocarcinoma, small intestine gastrointestinal stromal tumors (GISTs), large intestine/colon carcinoma, large intestine adenocarcinoma, soft tissue angiosarcoma, soft tissue Ewing sarcoma, soft tissue hemangioendothelioma, soft tissue inflammatory myofibroblastic tumor, soft tissue leiomyosarcoma, soft tissue liposarcoma, soft tissue neuroblastoma, soft tissue paraganglioma, soft tissue perivascular epitheliod cell tumor, soft tissue sarcoma, soft tissue synovial sarcoma, stomach adenocarcinoma, stomach adenocarcinoma diffuse-type, stomach adenocarcinoma intestinal type, stomach adenocarcinoma intestinal type, stomach leiomyosarcoma, thymus carcinoma, thymus thymoma lymphocytic, thyroid papillary carcinoma, unknown primary adenocarcinoma, unknown primary carcinoma, unknown primary malignant neoplasm, lymphoid neoplasm, unknown primary melanoma, unknown primary sarcomatoid carcinoma, unknown primary squamous cell carcinoma, unknown undifferentiated neuroendocrine carcinoma, unknown primary undifferentiated small cell carcinoma, uterus carcinosarcoma, uterus endometrial adenocarcinoma, uterus endometrial adenocarcinoma endometrioid, uterus endometrial adenocarcinoma papillary serous, and uterus leiomyosarcoma.

In some embodiments, the subject is concurrently administered one or more additional anticancer agents with the ADCs described herein, or a pharmaceutically acceptable salt thereof. In some embodiments, the subject is concurrently receiving radiation therapy with the ADCs described herein, or a pharmaceutically acceptable salt thereof. In some embodiments, the subject is administered one or more additional anticancer agents after administration of the ADCs described herein, or a pharmaceutically acceptable salt thereof. In some embodiments, the subject receives radiation therapy after administration of the ADCs described herein, or a pharmaceutically acceptable salt thereof.

In some embodiments, the subject has discontinued a prior therapy, for example, due to unacceptable or unbearable side effects, wherein the prior therapy was too toxic, or wherein the subject developed resistance to the prior therapy.

Some embodiments provide a method for delaying or preventing a disease or disorder, comprising administering a therapeutically effective amount of an ADC as described herein, or a pharmaceutically acceptable salt thereof, and a vaccine against the disease or disorder, to a patient at risk for developing the disease or disorder. In some embodiments, the disease or disorder is cancer, as described herein. In some embodiments, the disease or disorder is a viral pathogen. In some embodiments, the vaccine is administered subcutaneously. In some embodiments, the vaccine is administered intramuscularly. In some embodiments, the ADC and the vaccine are administered via the same route (for example, the ADC and the vaccine are both administered subcutaneously). In some embodiments, the ADC, or a pharmaceutically acceptable salt thereof, and the vaccine are administered via different routes. In some embodiments, the vaccine and the ADC, or a pharmaceutically acceptable salt thereof, are provided in a single formulation. In some embodiments, the vaccine and the ADC, or a pharmaceutically acceptable salt thereof, are provided in separate formulations.

### Compostions and Methods of Administration

Some embodiments provide a composition comprising a distribution of ADCs, as described herein. In some embodiments, the composition comprises a distribution of ADCs, as described herein and at least one pharmaceutically acceptable carrier. In some embodiments, the route of administration is parenteral. Parenteral administration includes subcutaneous injections, intravenous, intramuscular, intrasternal injection or infusion techniques. In some embodiments, the compositions are administered parenterally. In one of those embodiments, the ADCs are administered intravenously. Administration is typically through any convenient route, for example by infusion or bolus injection.

Compositions of an ADC are formulated so as to allow the ADC to be bioavailable upon administration of the composition to a subject. Compositions can be in the form of one or more injectable dosage units.

Materials used in preparing the compositions can be non-toxic in the amounts used. It will be evident to those of ordinary skill in the art that the optimal dosage of the active ingredient(s) in the composition will depend on a variety of factors. Relevant factors include, without limitation, the type of animal (e.g., human), the particular form of the compound, the manner of administration, and the composition employed.

In some embodiments, the ADC composition is a solid, for example, as a lyophilized powder, suitable for reconstitution into a liquid prior to administration. In some embodiments, the ADC composition is a liquid composition, such as a solution or a suspension. A liquid composition or suspension is useful for delivery by injection and a lyophilized solid is suitable for reconstitution as a liquid or suspension using a diluent suitable for injection. In a composition administered by injection, one or more of a surfactant, preservative, wetting agent, dispersing agent, suspending agent, buffer, stabilizer and isotonic agent is typically included.

In some embodiments, the liquid compositions, whether they are solutions, suspensions or other like form, can also include one or more of the following: sterile diluents such as water for injection, saline solution, physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as amino acids, acetates, citrates or phosphates; detergents, such as nonionic surfactants, polyols; and agents for the adjustment of tonicity such as sodium chloride or dextrose. A parenteral composition is typically enclosed in ampoule, a disposable syringe or a multiple-dose vial made of glass, plastic or other material. In some embodiments, the sterile diluent comprises physiological saline. In some embodiments, the sterile diluent is physiological saline. In some embodiments, the composition described herein are liquid injectable compositions that are sterile.

The amount of the ADC that is effective in the treatment of a particular disorder or condition will depend on the nature of the disorder or condition, which is usually determined by standard clinical techniques. In addition, in vitro or in vivo assays are sometimes employed to help identify optimal dosage ranges. The precise dose to be employed in the compositions will also depend on the route of parenteral administration, and the seriousness of the disease or disorder, and should be decided according to the judgment of the practitioner and each subject's circumstances.

In some embodiments, the compositions comprise an effective amount of an ADC such that a suitable dosage will be obtained. Typically, this amount is at least about 0.01% of the ADC by weight of the composition.

In some embodiments, the compositions dosage of an ADC administered to a subject is from about 0.01 mg/kg to about 100 mg/kg, from about 1 to about 100 mg of a per kg or from about 0.1 to about 25 mg/kg of the subject's body weight. In some embodiments, the dosage administered to a subject is about 0.01 mg/kg to about 15 mg/kg of the subject's body weight. In some embodiments, the dosage administered to a subject is about 0.1 mg/kg to about 15 mg/kg of the subject's body weight. In some embodiments, the dosage administered to a subject is about 0.1 mg/kg to about 20 mg/kg of the subject's body weight. In some embodiments, the dosage administered is about 0.1 mg/kg to about 5 mg/kg or about 0.1 mg/kg to about 10 mg/kg of the subject's body weight. In some embodiments, the dosage administered is about 1 mg/kg to about 15 mg/kg of the subject's body weight. In some embodiments, the dosage administered is about 1 mg/kg to about 10 mg/kg of the subject's body weight. In some embodiments, the dosage administered is about 0.1 to about 4 mg/kg, about 0.1 to about 3.2 mg/kg, or about 0.1 to about 2.7 mg/kg of the subject's body weight over a treatment cycle.

The term “carrier” refers to a diluent, adjuvant or excipient, with which a compound is administered. Such pharmaceutical carriers are liquids. Water is an exemplary carrier when the compounds are administered intravenously. Saline solutions and aqueous dextrose and glycerol solutions are also useful as liquid carriers for injectable solutions. Suitable pharmaceutical carriers also include glycerol, propylene, glycol, or ethanol. The present compositions, if desired, will in some embodiments also contain minor amounts of wetting or emulsifying agents, and/or pH buffering agents.

In some embodiments, the ADCs are formulated in accordance with routine procedures as a composition adapted for intravenous administration to animals, particularly human beings. Typically, the carriers or vehicles for intravenous administration are sterile isotonic aqueous buffer solutions. In some embodiments, the composition further comprises a local anesthetic, such as lignocaine, to ease pain at the site of the injection. In some embodiments, the ADC and the remainder of the formulation are supplied either separately or mixed together in unit dosage form, for example, as a dry lyophilized powder or water free concentrate in a hermetically sealed container such as an ampoule or sachette indicating the quantity of active agent. Where an ADC is to be administered by infusion, it is sometimes dispensed, for example, with an infusion bottle containing sterile pharmaceutical grade water or saline. Where the ADCs are administered by injection, an ampoule of sterile water for injection or saline is typically provided so that the ingredients can be mixed prior to administration.

The compositions are generally formulated as sterile, substantially isotonic and in full compliance with all Good Manufacturing Practice (GMP) regulations of the U.S. Food and Drug Administration.

### EXAMPLES

### General Methods:

All commercially available anhydrous solvents were used without further purification. All commercially available reagents were used without further purification unless otherwise noted. Analytical thin layer chromatography (TLC) was performed on silica gel 60 F254 aluminum sheets or glass plates (EMD Chemicals, Gibbstown, NJ). Flash column chromatography was performed on a Biotage Isolera One™ flash purification system 20 or Biotage Selekt™ flash purification system (Charlotte, NC). UPLC-MS analysis was performed on one of four systems. UPLC-MS system 1: Waters single quad detector mass spectrometer interfaced to a Waters Acquity UPLC system equipped with a Waters Acquity UPLC BEH C18 2.1×50 mm, 1.7 μm, reversed-phase column. UPLC-MS system 2: Waters Xevo G2 TOF mass spectrometer interfaced to a Waters Acquity H-class Ultra Performance LC equipped with a C8 Phenomenex Synergi 2.0×150 mm, 4 μm, 80 Å reversed-phase column with a Waters 2996 Photodiode Array Detector. UPLC-MS system 3 (C18): Shimadzu LC-20 AD & MS 2020 interfaced with a diode array detector (DAD) and positive ESI mass spectrometer equipped with either a Luna-C18 2.0×30 mm, 3 μm particle size reversed-phase column maintained at 40° C. or a Kinetex-C18 2.1×30 mm, 5 μm reversed-phase column maintained at 40° C. UPLC-MS system 4 (C18): Agilent 1200 series LC system interfaced a diode array detector (DAD) and Agilent 6110B positive ESI quadrapole mass spectrometer equipped with a Kinetex-C18 2.1×50 mm, 5 μm reversed-phase column maintained at 40° C.

Compounds were eluted using one of Methods A-E, as described herein.

Method A—a linear gradient of 5-95% acetonitrile in water (1 mL/min) over 1.0 min, followed by isocratic flow of 95% acetonitrile to 1.80 min (1.0 mL/min) and column equilibration back to 5% acetonitrile to 2.20 min (1.2 mL/min). The water contained 0.037% TFA (v/v) and the acetonitrile contained 0.018% TFA (v/v). The column used was a Phenomenex Luna C18 2.0×30 mm, 3 μm reversed-phase column.

Method B—a linear gradient of 5-95% acetonitrile in water (1 mL/min) over 1.0 min, followed by isocratic flow of 95% acetonitrile to 1.80 min (1.0 mL/min) and column equilibration back to 5% acetonitrile to 2.20 min (1.2 mL/min). The water contained 0.05% TFA (v/v) and the acetonitrile contained 0.05% TFA (v/v). The column used was a Phenomenex Kinetex C18 2.1×30 mm, 5 μm reversed-phase column.

Method C—isocratic flow of 5% acetonitrile in water for 0.4 min, followed by a linear gradient of 5-95% acetonitrile in water to 3.0 min, followed by isocratic flow for 95% acetonitrile to 4.0 min and column equilibration back to 5% acetonitrile to 4.5 min. The flow rate was 1.0 mL/min and the water contained 0.05% TFA (v/v) and the acetonitrile contained 0.05% TFA (v/v). The column used was a Phenomenex Kinetex C18 2.1×30 mm, 5 μm reversed-phase column.

Method D—a linear gradient of 3-60% acetonitrile over 1.7 min, then 60-95% acetonitrile to 2.0 min, followed by isocratic flow of 95% acetonitrile to 2.5 min followed by column equilibration back to 3% acetonitrile. The flow rate was 0.6 mL/min and the water contained 0.1% (v/v) formic acid and the acetonitrile contained 0.1% (v/v) formic acid. The column used was either a Waters Acquity UPLC BEH C18 2.1×50 mm, 1.7 μm, reversed-phase column or a C8 Phenomenex Synergi 2.0×150 mm, 4 μm, reversed-phase column.

Method E—a linear gradient of 3-95% acetonitrile over 1.5 min, followed by isocratic elution of 95% acetonitrile to 2.4 min, followed by equilibration back to 3% acetonitrile. The flow rate was 0.6 mL/min and the water contained 0.1% (v/v) formic acid and the acetonitrile contained 0.1% (v/v) formic acid. The column used was either a Waters Acquity UPLC BEH C18 2.1×50 mm, 1.7 μm, reversed-phase column or a C8 Phenomenex Synergi 2.0×150 mm, 4 μm, reversed-phase column.

Unless otherwise specified, preparatory HPLC (PrepHPLC) was performed on one of two instruments using the procedures listed herein: (Method F) a Shimadzu LC-8a preparative HPLC with a Phenomenex Luna C-18 250×50 mm, 10 μm using water/acetonitrile mobile phase with 0.09% (v/v) TFA at a flow rate of 80 mL/min or on a Teledyne ISCO ACCQPrep HP150 equipped with one of three Phenomemex preparatory HPLC columns: (i) (Method G) 10×250 mm Synergi C12, 4 μm, Max-RP 80 Å LC Column, (ii) (Method H) 21.2 ×250 mm Synergi C12, 4 μm, Max-RP 80 Å LC Column or (iii) (Method I) 30×250 mm Synergi C12, 4 μm, Max-RP 80 Å LC Column using acetonitrile/water mobile phases containing either 0.05% (v/v) trifluoroacetic acid or 0.1% (v/v) formic acid as additives.

NMR spectra were recorded on one of three instruments: Bruker Avance III HD (400 MHz), Varian 400-MR (400 MHz) or Bruker Avance NEO (400 MHz).

### Example 1

### Synthetic Procedures for Sting Agonists and Linkers

### Synthesis of (E)-1-(4-(5-carbamoyl-2-(1-ethyl-3-methyl-1H-pyrazole-5-carboxamido)-7-hydroxy-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide (Compound 1)

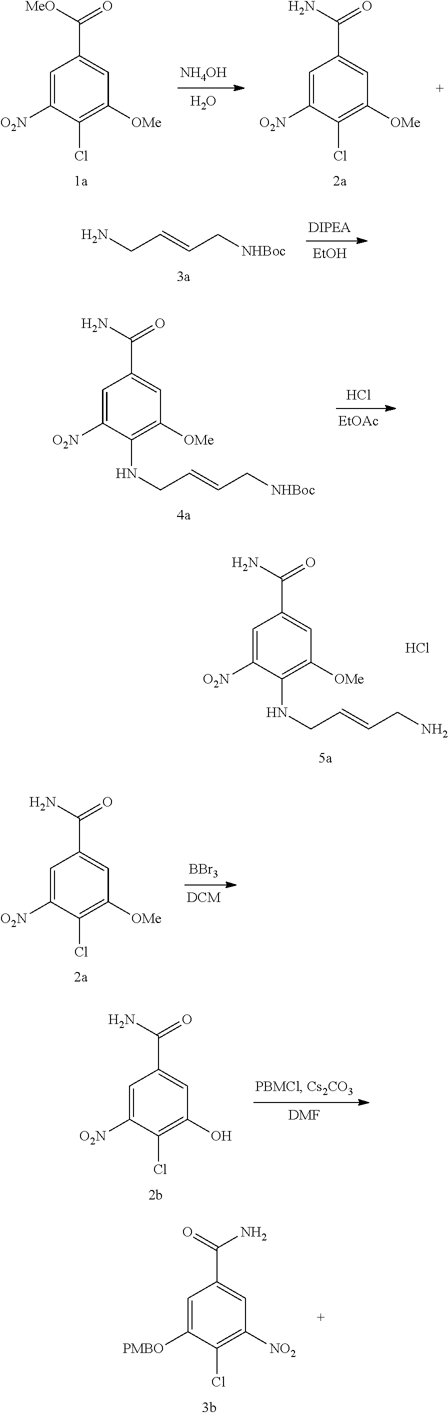

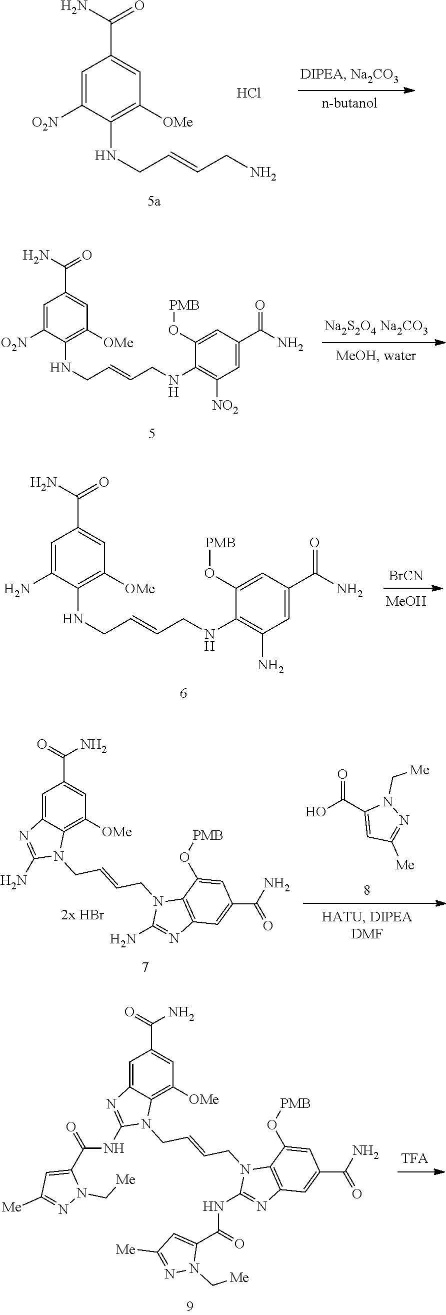

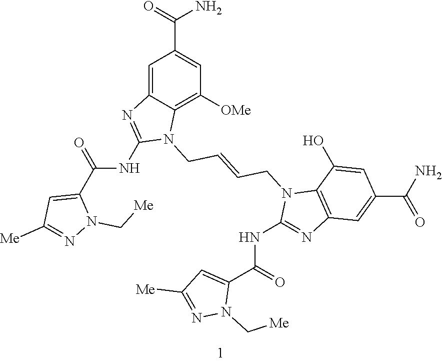 [SMILES: CCn1nc(C)cc1C(=O)Nc1nc2cc(C(N)=O)cc(O)c2n1C/C=C/Cn1c(NC(=O)c2cc(C)nn2CC)nc2cc(C(N)=O)cc(OC)c21]

### Synthesis of 4-chloro-3-met-5-nitrobenzamide (Compound 2a)

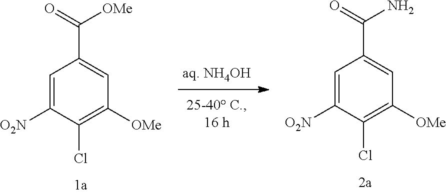 [SMILES: COC(=O)c1cc(OC)c(Cl)c([N+](=O)[O-])c1 ; COc1cc(C(N)=O)cc([N+](=O)[O-])c1Cl]

Compound 1a (methyl 4-chloro-3-methoxy-5-nitrobenzoate, 18 g, 73 mmol, 1 equiv.) was added into aqueous NH₄OH solution (300 mL, 28% NH₃ in H₂O) at 25° C. The reaction mixture was stirred at 40° C. for 16 hrs, during which time a precipitate was formed. The precipitate was collected by filtration, washed with water and dried in vacuo to give 2a (13 grams, 56 mmols, 77% yield) as a yellow solid. This product was used in subsequent steps without further purification. UPLC-MS (Method A, ESI+): m/z (M+H)⁺ 231.0 (theoretical); 231.2 (observed). HPLC retention time: 0.93 min. ¹H NMR (DMSO-d₆, 400 MHz): δ=8.29 (br s, 1H), 8.05 (d, J=2.0 Hz, 1H), 7.88 (d, J=1.6 Hz, 1H), 7.79 (br s, 1H), 4.02 (s, 3H).

### Synthesis of tert-butyl (E)-(4-((4-carbamoyl-2-methoxy-6-nitrophenyl)amino)but-2-en-1-yl)carbamate (Compound 4a)

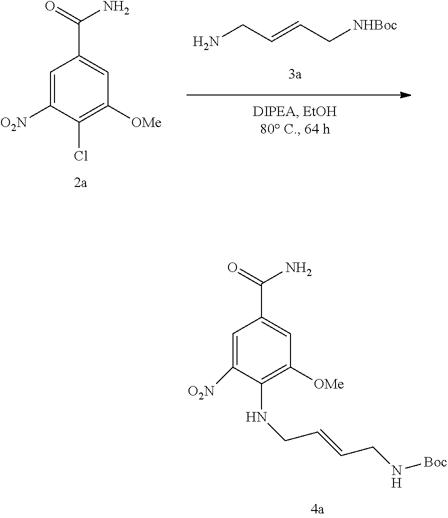 [SMILES: COc1cc(C(N)=O)cc([N+](=O)[O-])c1Cl ; CC(C)(C)OC(=O)NC/C=C/CN ; COc1cc(C(N)=O)cc([N+](=O)[O-])c1NC/C=C/CNC(=O)OC(C)(C)C]

To a solution of 2a (10 g, 43.4 mmol, 1 equiv.) in ethanol (EtOH, 200 mL) was added 3a (tert-butyl (E)-(4-aminobut-2-en-1-yl)carbamate, 9.69 g, 52.0 mmol, 1.2 equiv.) and N,N-diisopropylethylamine (DIPEA, 16.8 g, 130 mmol, 3 equiv.) at 25° C. The reaction mixture was stirred at 80° C. for 64 hours which point the precipitate was collected by filtration, washed with ethanol, and dried under high vacuum to give 4a (8 grams, 21 mmols, 48% yield) as a red solid. This product was used in subsequent steps without further purification. ¹H NMR (DMSO-d₆, 400 MHz): δ=8.18 (s, 1H), 8.01 (br s, 1H), 7.74 (br t, J=5.6 Hz, 1H), 7.55 (s, 1H), 7.31 (br s, 1H), 6.92 (br s, 1H), 5.53 (br s, 2H), 4.08 (br s, 2H), 3.87 (s, 3H), 3.47 (br s, 2H), 1.35 (s, 9H).

### Synthesis of Compound 5a

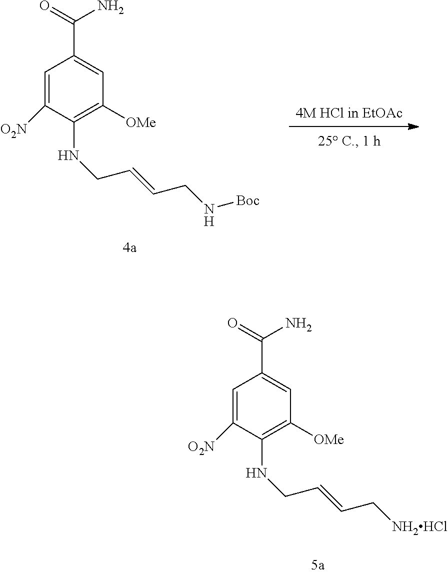

Compound 4a (8 g, 21.0 mmol, 1 equiv.) was added into a 4M solution of HCl in ethyl acetate (200 mL, 800 mmol HCl) at 25° C. The reaction mixture was stirred at 25° C. for 1 h. The precipitate was collected by filtration, washed with EtOAc and dried under high vacuum to give 5a as HCl salt (7.2 g, quantitative yield) as a red solid. This product was used in subsequent steps without further purification. ¹H NMR (DMSO-d₆, 400 MHz): δ=8.21 (d, J=1.6 Hz, 1H), 8.02 (br s, 4H), 7.59 (d, J=2.0 Hz, 1H), 7.34 (br s, 1H), 5.87 (td, J=5.6, 15.6 Hz, 1H), 5.67-5.56 (m, 1H), 4.17 (br d, J=5.6 Hz, 2H), 3.89 (s, 3H), 3.39 (br t, J=5.6 Hz, 2H).

### Synthesis of Compound 2b

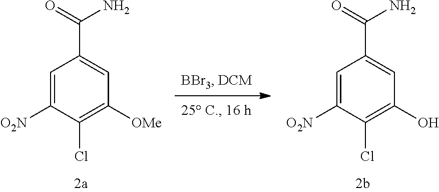 [SMILES: COc1cc(C(N)=O)cc([N+](=O)[O-])c1Cl ; NC(=O)c1cc(O)c(Cl)c([N+](=O)[O-])c1]

To a solution of compound 2a (4-chloro-3-methoxy-5-nitrobenzamide, 16 g, 69.4 mmol, 1 equiv.) in dichloromethane (DCM, 500 mL) was added a solution of boron tribromide (BBr₃, 1 M in DCM, 275 mL, 4 equiv.) dropwise at 20° C. under nitrogen. The reaction mixture was stirred at 20° C. for 16 h, upon which LC-MS analysis (Method B) showed the reaction was complete. The reaction mixture was poured into ice water (2 L) and stirred vigorously for 20 min. The resulting suspension was filtered and the filtrate was extracted with ethyl acetate (2×300 mL), dried over anhydrous Na₂SO₄, filtered and concentrated in vacuo to give a crude product. The crude product (9 g) was dissolved in DMF (30 mL) and purified by reversed-phase flash chromatography on a Biotage Isolera One (330 gram Agela C18 column (20-35 μm particle size), utilizing water/acetonitrile with 0.09% (v/v) TFA eluting with a gradient of 20-40% acetonitrile over 20 min followed by 40-45% acetonitrile at 35 min to give 2b (6 grams, 27.7 mmols, 40% yield) as an off-white solid. LCMS (Method B, ESI+): m/z [M+H]⁺ 217.0 (theoretical); 217.2 (observed). HPLC retention time: 0.84 min.

### Synthesis of Compound 3b

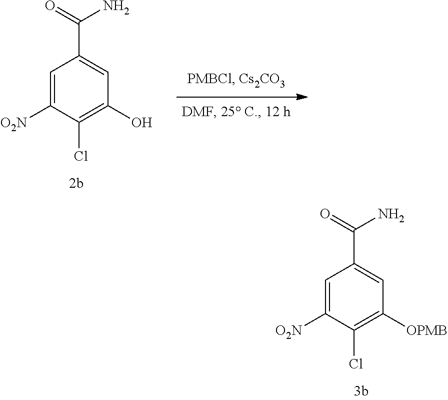

To a solution of 2b (4.5 g, 20.8 mmol, 1 equiv.) in dimethylformamide (DMF, 20 mL) was added 1-(chloromethyl)-4-methoxybenzene (PMBCl, 3.42 g, 21.8 mmol, 1.05 equiv.) and cesium carbonate (Cs₂CO₃, 7.45 g, 22.9 mmol, 1.1 equiv.), the reaction mixture was stirred at 25° C. for 12 h, upon which LC-MS analysis (Method B) showed the reaction was complete. The reaction mixture was poured into ice water, and the precipitate was filtered and dried to give 3b (6.4 grams, 19.0 mmols, 91% yield)) as a light yellow solid. This product was used in subsequent steps without further purification. LC-MS (Method B, ESI+): m/z [M+H]⁺: 337.1 (theoretical); 337.2 (observed). HPLC Retention Time: 1.11 min.

### Synthesis of Compound 5

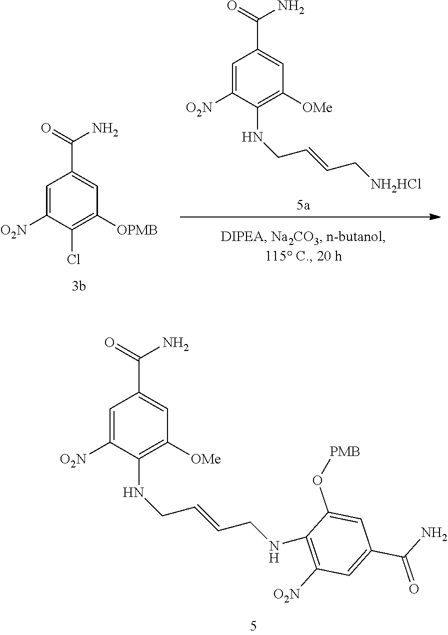

A solution of 5a (762 mg, 2.16 mmol, 1.2 equiv.) in n-butanol (10 mL) was added to a vial, followed by the addition of DIPEA (1.11 g, 8.62 mmol, 4.8 equiv.) and sodium bicarbonate (457 mg, 4.31 mmol, 2 equiv.). The vial was sealed and the reaction mixture was stirred at 20° C. for 10 min. This was followed by the addition of 3b (600 mg, 1.78 mmol, 2.4 equiv.), and the reaction mixture was stirred at 115° C. for 20 hours upon which time UPLC-MS analysis (Method B) showed the reaction was complete. Four additional vials were set up as described above. All five reaction mixtures were combined at the end of the reaction. The resulting combined reaction mixture was cooled to 20° C. and diluted with MeCN (180 mL). The solid material in the reaction mixture was filtered and rinsed with MeCN (80 mL) to give a dark red solid. The solid was then washed with water and dried under high vacuum to give 5 (2.7 grams, 4.65 mmols, 52% yield) as a brick-red solid. This product was used in subsequent steps without further purification. ¹H NMR (400 MHz, DMSO-d₆): δ=8.17 (dd, J=1.9, 7.8 Hz, 2H), 8.08-7.96 (m, 2H), 7.77-7.63 (m, 3H), 7.51 (d, J=1.8 Hz, 1H), 7.37 (d, J=8.6 Hz, 2H), 7.33 (br s, 2H), 6.92 (d, J=8.6 Hz, 2H), 5.57-5.42 (m, 2H), 5.04 (s, 2H), 4.01 (q, J=5.8 Hz, 4H), 3.79 (s, 3H), 3.74 (s, 3H).

### Synthesis of Compound 6

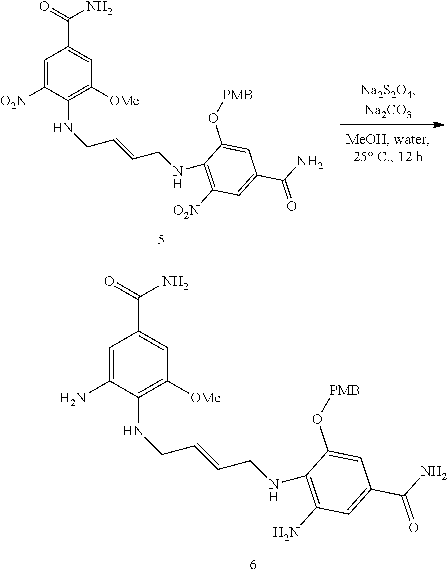

To a solution of 5 (2 g, 3.45 mmol, 1 equiv.) in a 1:1 (v/v) mixture of methanol and water (160 mL) was added Na₂CO₃ (10.95 g, 103 mmol, 30 equiv.) and sodium dithionite (Na₂S₂O₄, 8.40 g, 48.2 mmol, 14 equiv.). The resulting red reaction mixture was stirred at 25° C. for 12 h, upon which the red mixture turned into a pale yellow color, and UPLC-MS analysis (Method B) showed the reaction was complete. The reaction mixture was filtered, and the filtrate was concentrated and diluted with water. The mixture was extracted with EtOAc and the organic layer was concentrated to give 6 (1.0 grams, 1.81 mmols, 52% yield) as an off-white solid. This product was used in subsequent steps without further purification. ¹H NMR (400 MHz, DMSO-d₆): δ=7.61 (br s, 2H), 7.37 (d, J=8.6 Hz, 2H), 6.97 (br s, 2H), 6.94 (s, 1H), 6.93-6.90 (m, 2H), 6.86 (s, 2H), 6.77 (d, J=1.8 Hz, 1H), 5.71-5.53 (m, 2H), 4.98 (s, 2H), 4.65 (br d, J=12.6 Hz, 4H), 3.74 (s, 3H), 3.71 (s, 3H), 3.49 (br s, 4H).

### Synthesis of Compound 7

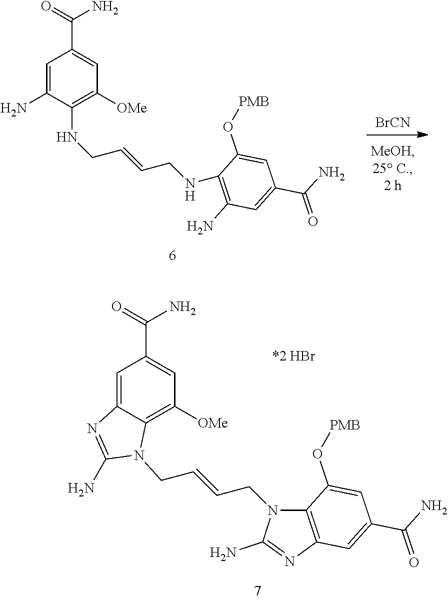

To a solution of 6 (1.4 g, 2.69 mmol, 1 equiv.) in methanol (20 mL) was added cyanogen bromide (BrCN, 1.71 g, 16.1 mmol, 6 equiv.). The reaction mixture was stirred at 25° C. for 2 h, during which time a precipitate was observed. LC-MS analysis (Method C) showed the reaction was complete. The solid was collected by filtration, washed with ethanol and petroleum ether to give 7 (1.2 g, 1.64 mmols, 61% yield) as a light yellow solid. This product was used in subsequent steps without further purification. LC-MS (Method C, ESI+): m/z [M+H]⁺ 571.2 (theoretical); 571 (observed). HPLC retention time: 1.634 min. ¹H NMR (400 MHz, DMSO-d₆): δ=12.94 (br s, 2H), 8.63 (br d, J=12.8 Hz, 4H), 8.08 (br s, 2H), 7.62-7.52 (m, 3H), 7.47 (br s, 2H), 7.38 (s, 1H), 7.24 (d, J=8.6 Hz, 2H), 6.84 (d, J=8.6 Hz, 2H), 5.81-5.69 (m, 1H), 5.57 (td, J=5.4, 15.5 Hz, 1H), 5.07 (s, 2H), 4.80 (br t, J=6.6 Hz, 4H), 3.74 (s, 3H), 3.69 (s, 3H).

### Synthesis of Compound 9

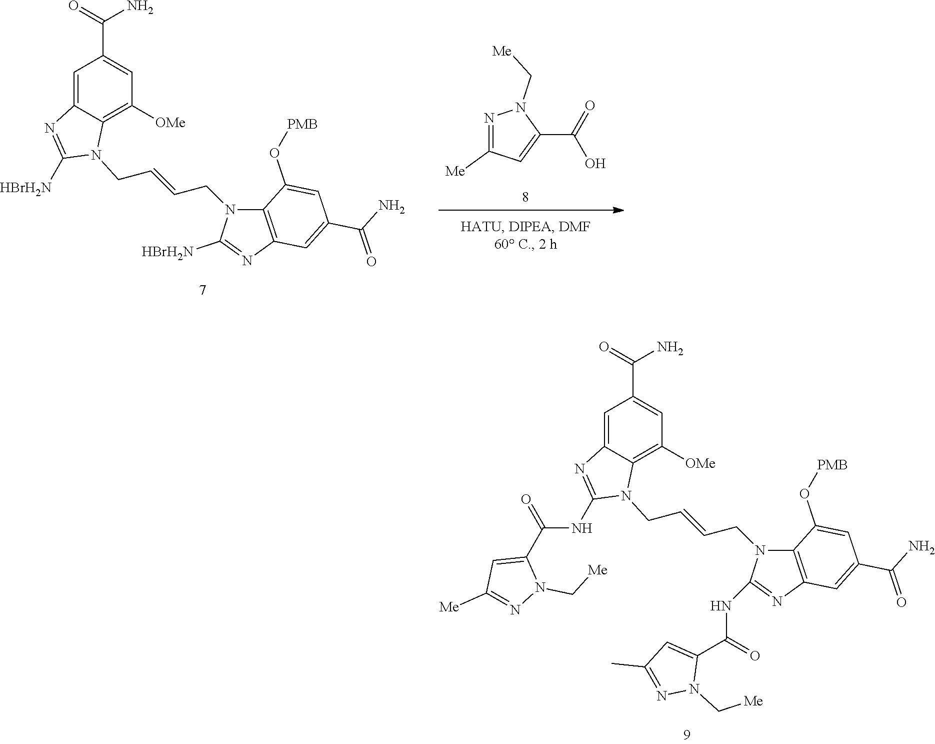

To a solution of compound 8 (1-ethyl-3-methyl-1H-pyrazole-5-carboxylic acid, 331 mg, 2.15 mmol, 2.1 equiv.) in dimethylformamide (DMF, 3 mL) was added 1-[bis(dimethylamino)methylene]-1H-1,2,3-triazolo[4,5-b]pyridinium 3-oxid hexafluorophosphate (HATU, 973 mg, 2.56 mmol, 2.5 equiv.) and the reaction mixture was stirred at 60° C. for 10 min. A solution of DIPEA (596 mg, 4.61 mmol, 4.5 equiv.) and 7 (750 mg, 1.02 mmol, 1 equiv.) in DMF (1 mL) was then added to the reaction mixture, which was stirred at 60° C. for 2 h, upon which LC-MS analysis (Method B) showed the reaction was complete. The reaction mixture was poured into ice water, the solid was collected by filtration and dried to give a crude product. The crude product was used in the next step without further purification. LC-MS (Method B, ESI+): m/z [M+H]⁺ 843.4 (theoretical); 843.4 (observed). HPLC Retention Time: 1.062 min.

### Synthesis of Compound 1

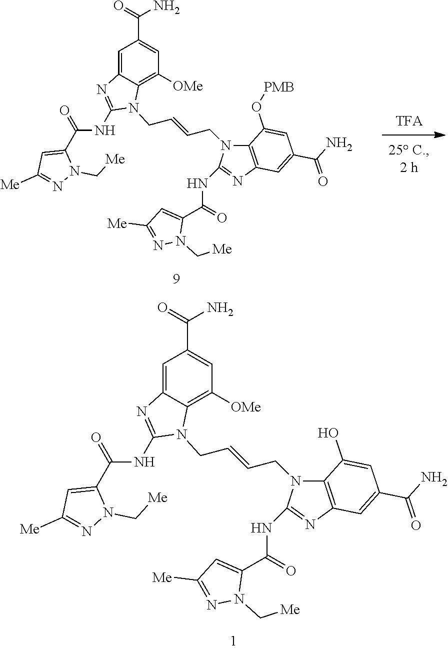

Compound 9 (700 mg, 0.83 mmol) was added to a glass vial containing trifluoroacetic acid (TFA, 3 mL), and the resulting mixture was stirred at 25° C. for 2 h, upon which LC-MS analysis showed the reaction was complete. The TFA was removed in vacuo and the residue was dissolved in DMSO and acetonitrile and purified by preparatory HPLC (Method F) to give 1 (40 mg, 0.055 mmols, 7% yield over 2 steps) as an off-white solid. LCMS (Method B, ESI+): m/z [M+H]⁺ 723.3 (theoretical); 723.1 (observed); [M+H]⁺, HPLC retention time: 2.04 min. ¹H NMR (400 MHz, DMSO-d₆): δ=13.00-12.51 (m, 2H), 10.41 (s, 1H), 7.96 (br s, 1H), 7.81 (br s, 1H), 7.63 (s, 1H), 7.43 (s, 1H), 7.37-7.28 (m, 2H), 7.22 (br s, 1H), 7.14-7.07 (m, 1H), 6.51 (br d, J=11.0 Hz, 2H), 5.97-5.75 (m, 2H), 4.91 (br dd, J=3.5, 16.3 Hz, 4H), 4.51 (br d, J=3.3 Hz, 4H), 3.77 (s, 3H), 2.10 (d, J=6.0 Hz, 6H), 1.25 (dt, J=3.6, 6.9 Hz, 6H).

### Synthesis of (2S,3S,4S,5R,6S)-6-(4-((((2-((((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)carbonyl)(methyl)amino)ethyl)(methyl)carbamoyl)oxy)methyl)-3-(3-(3-(2,5-dioxo-2,5-dihydro-1H-pyrrol-1-yl)propanamido)propanamido)phenoxy)-3,4,5-trihydroxytetrahydro-2H-pyran-2-carboxylic acid (Compound 11)

### Synthesis of Compound 10b

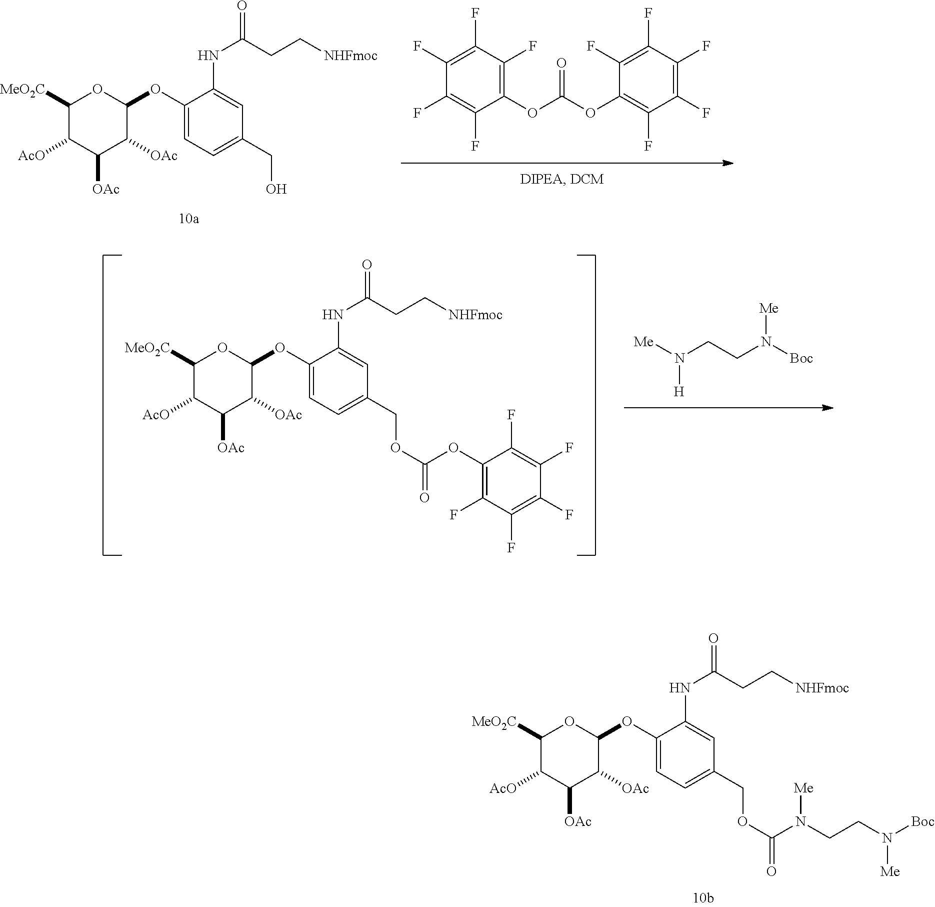

Compound 10a was prepared as previously reported (ACS Med. Chem. Lett. 2010, 1, 6, 277-280).

An oven-dried 4 mL glass vial was charged with 10a (150 mg, 0.20 mmol, 1 equiv.) and pentafluorophenyl carbonate (88 mg, 0.22 mmol, 1.1 equiv.), DMF (1 mL) and DIPEA (0.15 mL, 0.86 mmol, 4.3 equiv.). The reaction mixture was stirred at room temperature for 30 minutes upon which a light pink homogenous solution was observed. Tert-butyl methyl(2-(methylamino)ethyl)carbamate (50 uL, 0.27 mmol, 1.3 equiv.) was added to the solution, which resulted in the reaction mixture turning to a light yellow color. The reaction mixture was stirred at room temperature overnight. The reaction mixture was diluted with water (50 mL), transferred to a separatory funnel and extracted with EtOAc (3×50 mL). The organic layers were collected and combined, washed with 1M HCl, dried with MgSO₄, filtered and the solvent removed in vacuo. The resulting solid was purified by flash column chromatography (25 g SiO₂ column, eluting with 0-25% MeOH in DCM) to yield 10b as a light yellow solid (70.4 mg, 0.073 mmol, 36% yield). UPLC-MS (Method E, ESI+) m/z [(M-Boc)+2H]⁺: 863.33 (theoretical); 863.14 (observed). HPLC retention time: 1.54 min.

### Synthesis of Compound 10c

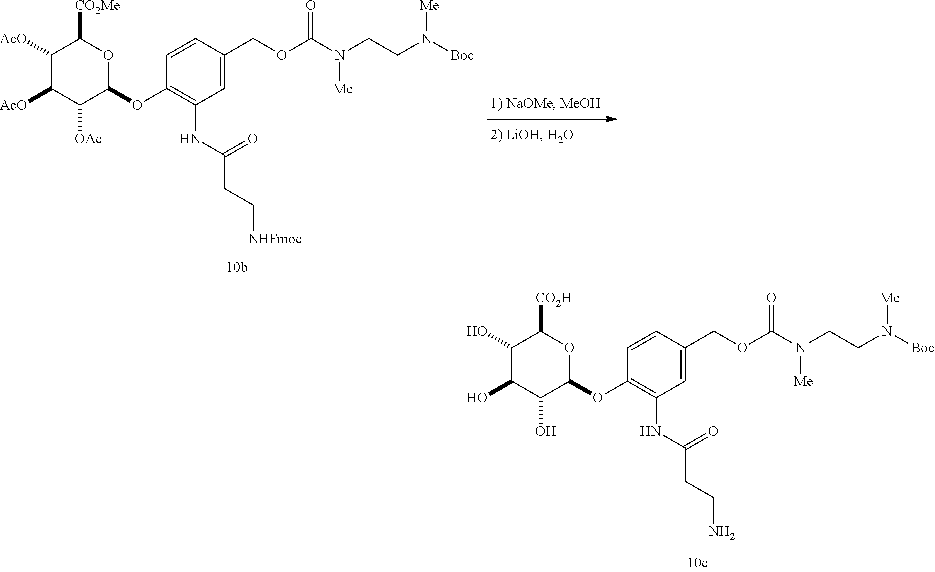

Compound 10b (70.4 mg, 0.073 mmol, 1 equiv.) was transferred as a solution in MeOH to an oven-dried 4 mL glass vial equipped with a magnetic stir bar. The MeOH was removed under vacuum and the vial back-filled with argon. To the vial, under Ar, was added MeOH (0.5 mL) and the resulting solution was cooled to 0° C. and sodium methoxide (0.5 M solution in MeOH, 150 uL, 0.075 mmol, 1 equiv.) was added. The reaction was monitored by LC-MS (Method D) and upon complete removal of all three acetate groups, lithium hydroxide (1M in water, 0.225 mL, 0.225 mmol, 3 equiv.) was added and the reaction mixture was stirred at room temperature for 30 min. DMSO (0.5 mL) and glacial acetic acid (50 uL) were added to the reaction mixture, yielding a homogenous solution. The crude product was purified by preparatory HPLC (Method H, 5-40% MeCN in water with 0.05% TFA as mobile phase additive) to give 10c as a white solid (16.8 mg, 0.028 mmol, 38% yield). UPLC-MS (Method D, ESI+): m/z [M+H]⁺ 601.26 (theoretical); 601.15 (observed). HPLC retention time: 1.09 min.

### Synthesis of Compound 10d

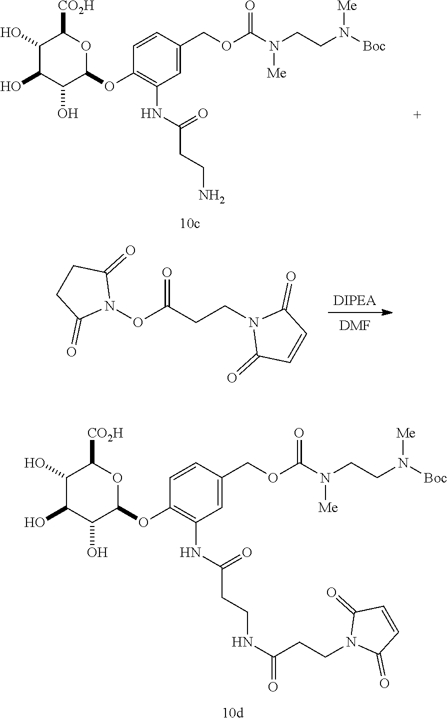

Compound 10c (16.8 mg, 0.028 mmol, 1 equiv.) was added to an oven-dried 4 mL glass vial equipped with a magnetic stir bar as a solution in MeOH. The MeOH was removed under vacuum and the vial filled with argon. To the vial was added 3-(maleimido)propionic acid N-hydroxysuccinimide ester (MP-OSu, 16 mg, 0.06 mmol, 2 equiv.) followed by DMF (0.5 mL) and DIPEA (50 uL, 0.28 mmol, 10 equiv.). After 15 minutes, DMSO (0.5 mL) and glacial acetic acid (100 uL) were added and the crude product purified by preparatory HPLC (Method H, 10-60% MeCN in water with 0.05% TFA as mobile phase additive) to give 10d as a white solid (15 mg, 0.020 mmol, 71% yield). UPLC-MS (Method A, ESI+): m/z [M+H]⁺: 752.29 (theoretical); 752.26 (observed). HPLC retention time: 1.27 min.

### Synthesis of Compound 10

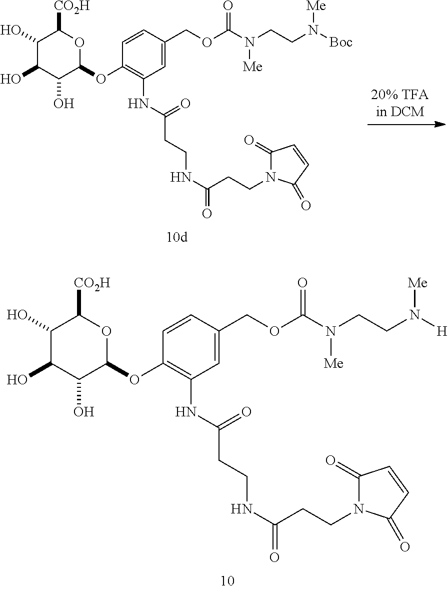 [SMILES: CN(CCN(C)C(=O)OC(C)(C)C)C(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1 ; CNCCN(C)C(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1]

Compound 10d (15 mg, 0.020 mmols, 1 equiv.) was dissolved in 20% (v/v) TFA in DCM (1 mL) and transferred to a 4 mL glass vial equipped with a magnetic stir bar. The vial was left uncapped and the reaction progress was monitored by LC-MS. After 2h, the solvent was removed in vacuo to give 10 as a white solid (13 mg, 0.02 mmol, quantitative yield) which was used in subsequent steps without any further purification. UPLC-MS (Method D, ESI+): m/z [M+H]⁺: 652.24 (theoretical); 652.45 (observed). HPLC retention time: 0.69 min.

### Synthesis of Compound 11

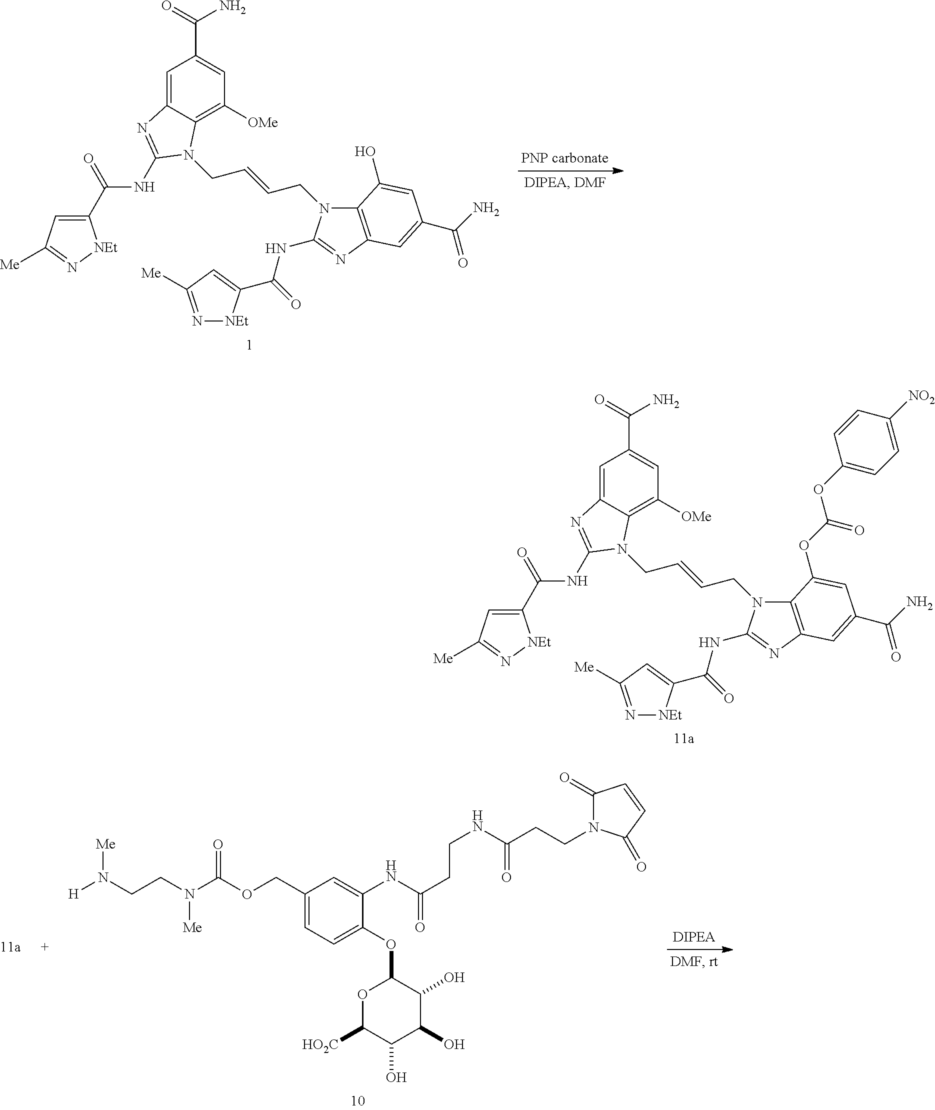

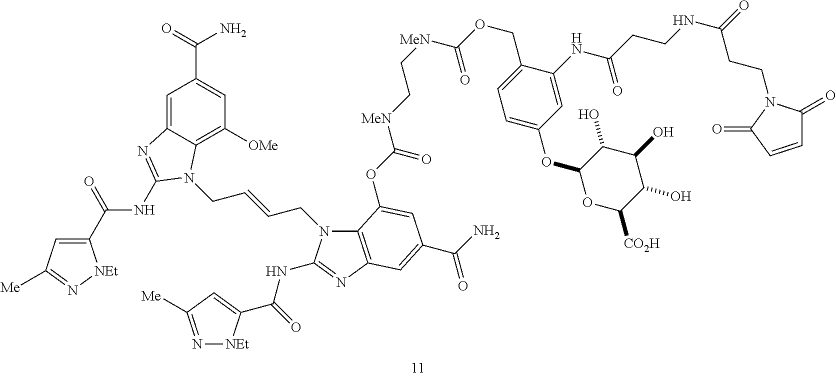

To an oven-dried 4 mL glass vial was added Compound 1 (9.5 mg, 0.010 mmol, 1 equiv.) followed by DMF (0.5 mL), p-nitrophenyl carbonate (9.0 mg, 0.030 mmol, 3 equiv.) and DIPEA (20 uL, 0.115 mmol, 11.5 equiv.). The reaction mixture was stirred at room temperature for 1 hour at which point full conversion to 11a was confirmed by UPLC-MS analysis (Method D). Compound 10 (20 mg, 0.031 mmol, 3.1 equiv.) was added in a single portion to the reaction mixture which was stirred at room temperature for 2 h. Glacial acetic acid (20 uL) was added and the crude product purified by preparatory HPLC (Method H, 0-45% MeCN in water with 0.05% TFA as mobile phase additive). The fractions containing 11 were combined and the solvent was removed via lyophilization to give 11 (6.31 mg, 0.0039 mmol, 39% yield). Compound 1 was also recovered (2.81 mg, 0.0030 mmol, 30% recovery) as a white fluffy solid. UPLC-MS (Method D, ESI+): m/z [M+H]⁺: 1400.52 (theoretical); 1400.25 (observed) & [M+2H]²+=701.43 (observed). HPLC retention time: 1.28 min.

### Synthesis of (E)-1-(4-(5-carbamoyl-2-(1-ethyl-3-methyl-1H-pyrazole-5-carboxamido)-7-methoxy-1H-benzo[d]imidazol-1-yl)but-2-en-1-yl)-7-(3-(3-(2,5-dioxo-2,5-dihydro-1H-pyrrol-1-yl)-N-methylpropanamido)propoxy)-2-(1-ethyl-3-methyl-1H-pyrazole-5-carboxamido)-1H-benzo[d]imidazole-5-carboxamide (Compound 12)

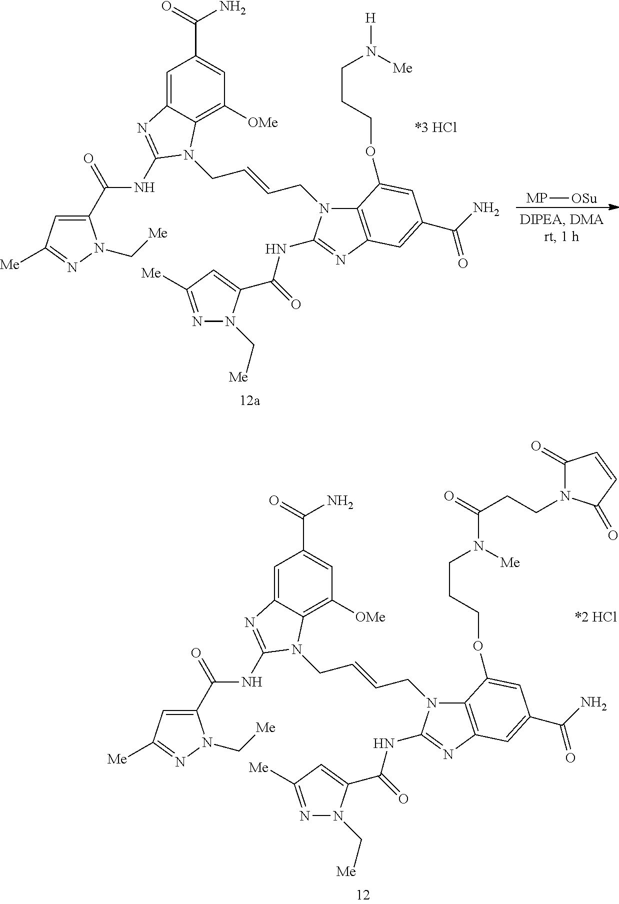

Compound 12a was prepared as previously reported (WO2017/175147, example 40, page 292).

To a solution of 12a (28.7 mg, 0.032 mmol, 1.0 equiv.) in DMA (635 μL) was added MP-OSu (15.9 mg, 0.0596 mmol, 1.9 equiv.), and DIPEA (35 μL, 0.199 mmol, 6.2 equiv.). The reaction mixture was stirred for 1 h at room temperature. Upon completion, the solution was concentrated under reduced pressure and the crude product was purified by preparatory HPLC (Method G, 20-50-95% MeCN in water with 0.1% formic acid as mobile phase additive) to yield 12 (46% yield, 17.8 mg, 0.0152 mmol). UPLC-MS (Method D, ESI+): m/z [M+H]⁺ 945.40 (theoretical); 945.72 (observed). HPLC retention time: 1.79 min.

### Synthesis of (2S,3S,4S,5R,6S)-6-(4-((((3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)(methyl)carbamoyl)oxy)methyl)-3-(3-(3-(2,5-dioxo-2,5-dihydro-1H-pyrrol-1-yl)propanamido)propanamido)phenoxy)-3,4,5-trihydroxytetrahydro-2H-pyran-2-carboxylic acid (Compound 13)

### Synthesis of Compounds 13a and 13b

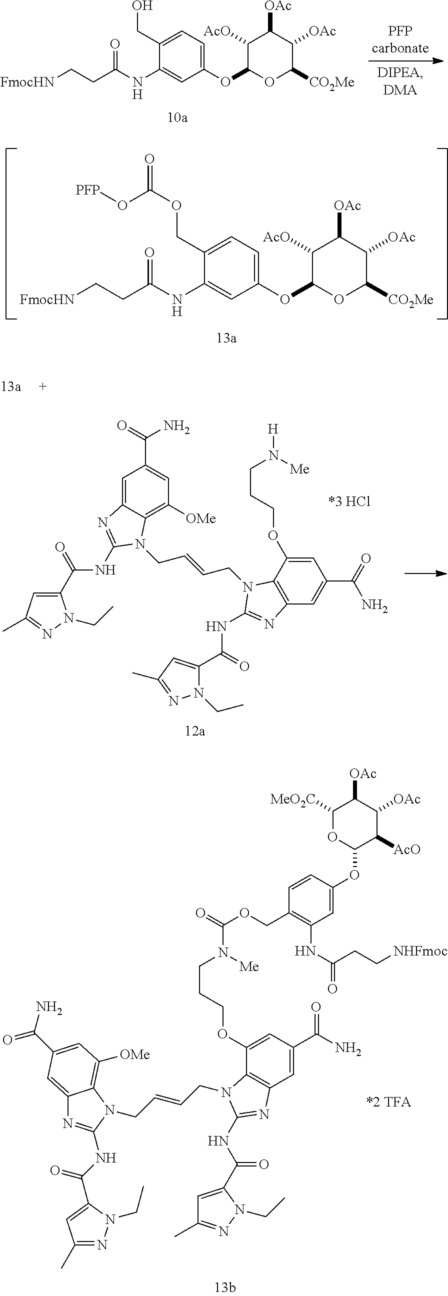

Compound 10a (13 mg, 0.017 mmol) was dissolved in DMA (87 μL). To this solution was added pentafluorophenyl carbonate (13.7 mg, 0.035 mmol), and DIPEA (14 μL, 0.078 mmol). The mixture was stirred for 30 min at room temperature. Upon full conversion to intermediate 13a, this solution was transferred to a second vial containing 12a (10.6 mg, 0.012 mmol). The reaction mixture was stirred for 18 h at room temperature. The reaction was then diluted with water and extracted three times with EtOAc (20 mL×3). The combined organic layers were then washed with 1M HCl. The organic layers were combined, dried with MgSO₄, filtered, and concentrated in vacuo. The product was purified by preparatory HPLC (Method H, 5-50-95% MeCN in water using 0.05% TFA as mobile phase additive) to yield compound 13b as a trifluoroacetate salt (10.0 mg, 0.0056 mmol, 48% yield). UPLC-MS (Method D, ESI+): m/z [M+H]⁺=1568.60 (theoretical); 1568.95 (observed). HPLC retention time: 1.70 min.

### Synthesis of Compound 13c

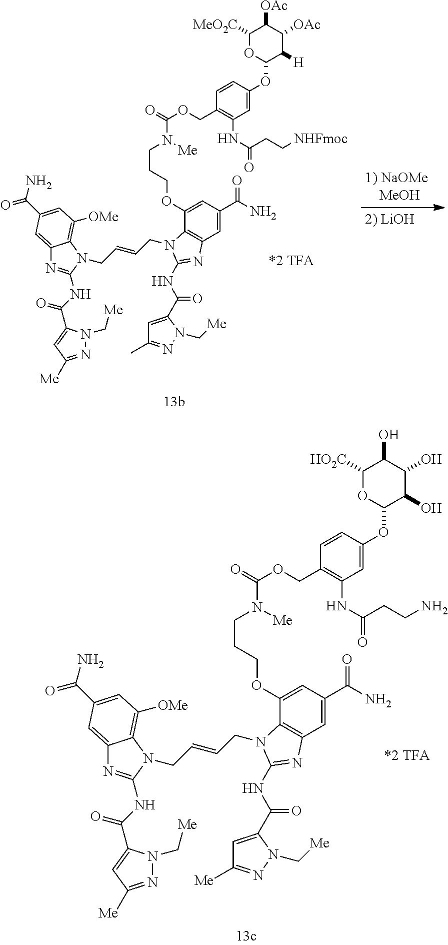

To a dry, well purged glass vial was added compound 13b (10.0 mg, 0.0056 mmol) in anhydrous methanol (40 μL). The solution was cooled in an ice bath, and NaOMe (0.5 M in MeOH, 11.13 μL) was added. After about 1 h, 1 M aqueous LiOH (17 μL, 0.017 mmols, 3 equiv.) solution was added. Significant white precipitate formed upon the addition of the LiOH solution. After 1 hr, glacial acetic acid (12 μL) was added, and the solvents were removed in vacuo. The crude product was purified by preparatory HPLC (Method G, 20-60-95% MeCN in water, with 0.05% TFA as mobile phase additive) to yield compound 13c as trifluoroacetate salt (4.13 mg, 0.0029 mmol, 52% yield). UPLC-MS (Method D, ESI+): m/z [M+H]⁺=1206.49 (theoretical); 1206.50 (observed). HPLC retention time: 1.45 min.

### Synthesis of Compound 13

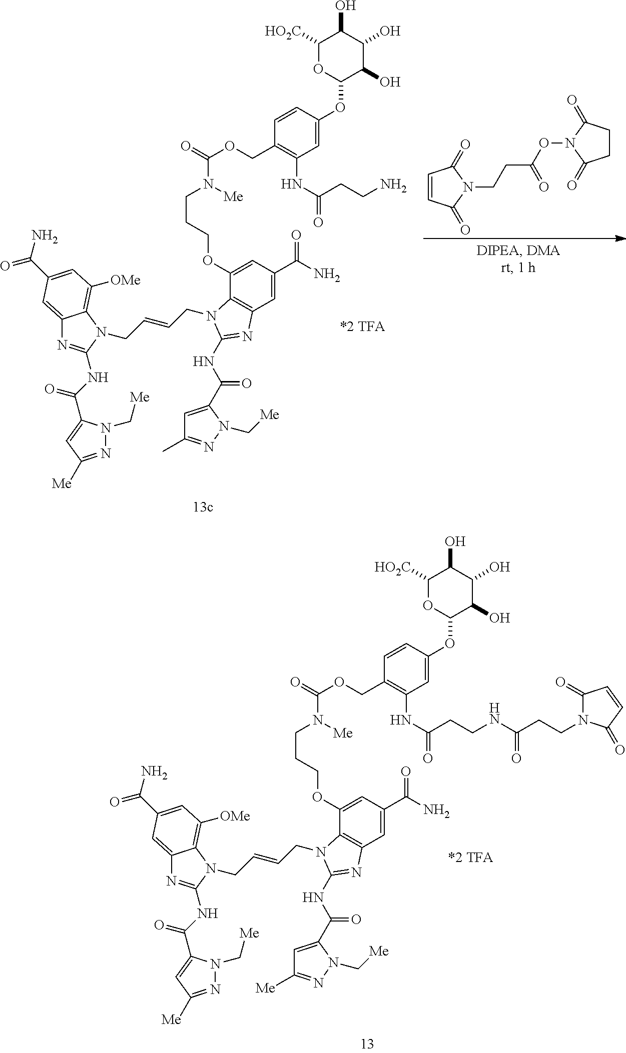 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)OCc3ccc(O[C@@H]4O[C@H](C(=O)O)[C@@H](O)[C@H](O)[C@H]4O)cc3NC(=O)CCN)c21 ; O=C(CCN1C(=O)C=CC1=O)ON1C(=O)CCC1=O ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)OCc3ccc(O[C@@H]4O[C@H](C(=O)O)[C@@H](O)[C@H](O)[C@H]4O)cc3NC(=O)CCNC(=O)CCN3C(=O)C=CC3=O)c21]

Compound 13c (4.13 mg, 0.00342 mmol, 1.0 equiv.) was dissolved in DMA (68 μL) in a glass vial under argon. MP-OSu (1.82 mg, 0.00685 mmol, 2 equiv.) and DIPEA (3.0 μL, 0.0171 mmol, 5 equiv.) were added and the reaction mixture was stirred for 1 h at RT. Glacial acetic acid (3.0 μL) was added, and the crude product purified by preparatory HPLC (Method G, 10-60-95% MeCN in water using 0.1% formic acid as mobile phase additive) to yield 13 as trifluoroacetate salt (5.43 mg, 0.0034 mmol, 93% yield). UPLC-MS (Method E, ESI+): m/z [M+H]⁺=1357.52 (theoretical); 1357.82 (observed). HPLC retention time: 1.54 min.

### Synthesis of 4-((S)-2-((S)-2-(3-(2,5-dioxo-2,5-dihydro-1H-pyrrol-1-yl)propanamido)-3-methylbutanamido)propanamido)benzyl (3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)(methyl)carbamate (Compound 14)

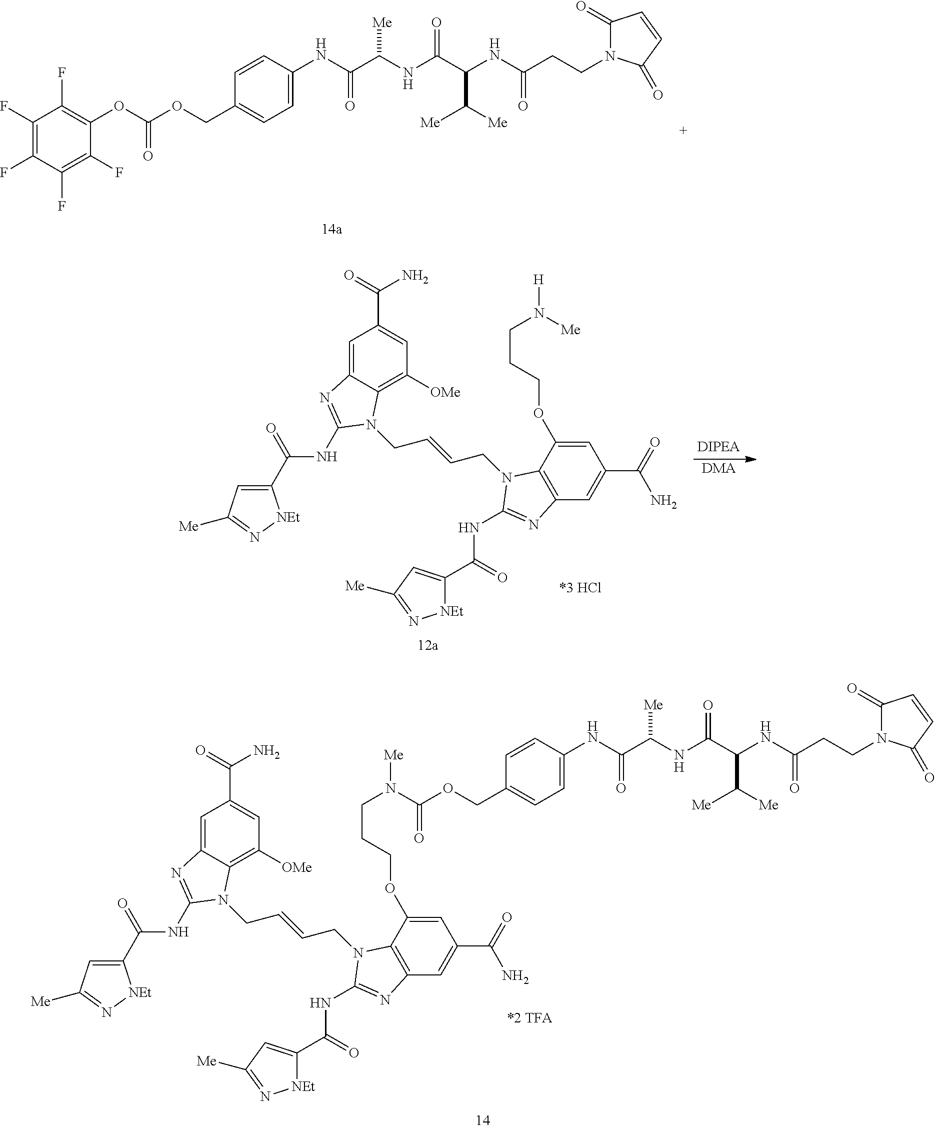

To a dry glass vial charged with compound 12a (2.6 mg, 0.0033 mmol) was added DMA (66 μL) followed by MP-Val-Ala-PAB-Opfp (14a, 3.2 mg, 0.049 mmol, 15 equiv.) and DIPEA (2.8 μL, 0.016 mmol, 4.9 equiv.). The reaction mixture was stirred for 30 minutes at RT and then glacial acetic acid (2.85 μL) was added, and the crude product purified by preparatory HPLC (Method G, 30-60-95% MeCN in water, with 0.1% formic acid as mobile phase additive), to yield compound 14 as trifluoroacetate salt (4.0 mg, 0.0027 mmol, 82% yield). UPLC-MS (Method D, ESI+): m/z [M+H]⁺=1264.56 (theoretical); 1264.85 (observed). HPLC retention time: 1.75 min.

### Synthesis of (E)-1-(4-(5-carbamoyl-2-(1-ethyl-3-methyl-1H-pyrazole-5-carboxamido)-7-hydroxy-1H-benzo[d]imidazol-1-yl)but-2-en-1-yl)-7-(3-(3-(2,5-dioxo-2,5-dihydro-1H-pyrrol-1-yl)-N-methylpropanamido)propoxy)-2-(1-ethyl-3-methyl-1H-pyrazole-5-carboxamido)-1H-benzo[d]imidazole-5-carboxamide (Compound 15)

### Synthesis of Compound 15b

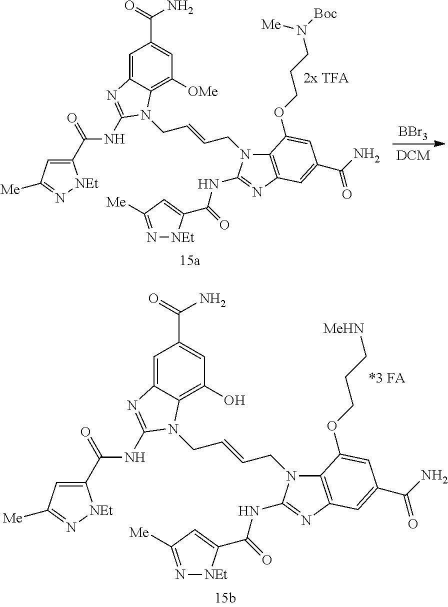

Compound 15a was prepared as previously reported (WO2017/175147, page 292)

To a dry glass vial containing compound 15a (31.4 mg, 0.0280 mmol) in DCM (280 μL) was added boron tribromide (BBr₃, 1M in DCM, 168 μL, 0.168 mmol, 6 equiv.) dropwise. The reaction mixture was stirred at 40° C. for 18 h. The reaction mixture was cooled to RT and cold water (170 μL) was slowly added. The resulting mixture was concentrated in vacuo and purified by preparatory HPLC (20-50-95%, 0.1% formic acid in acetonitrile, Method G). Fractions containing the desired product were combined and solvent removed via lyophilization to yield compound 15b as the formate salt (17% yield, 4.36 mg, 0.0047 mmol). UPLC-MS (Method D, ESI+): m/z [M+H]⁺=780.36 (theoretical); 780.38 (observed). HPLC retention time: 1.33 min.

### Synthesis of Compound 15

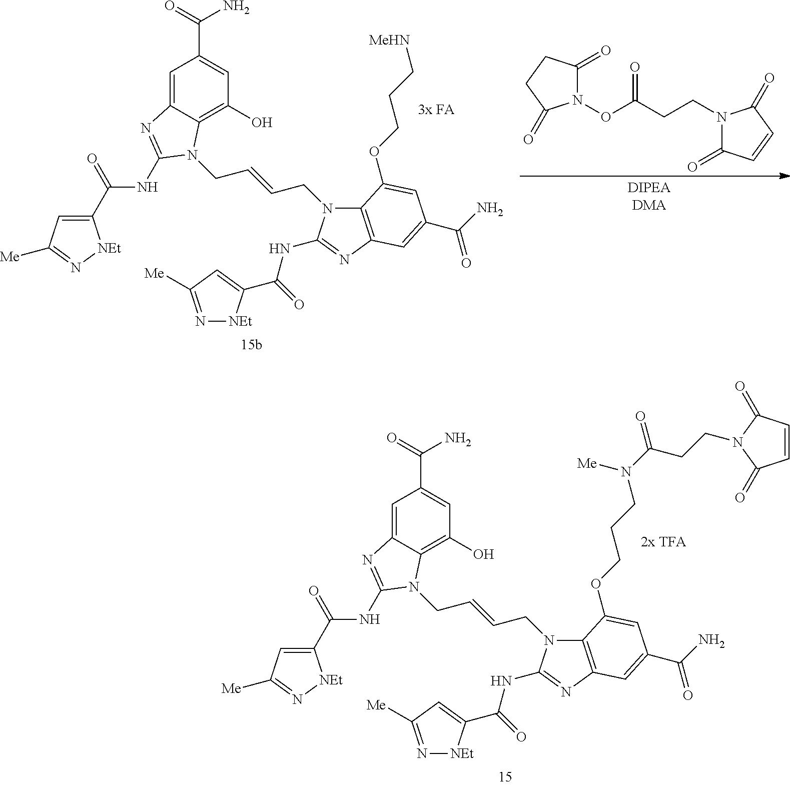

To a dry 4 mL vial containing 2,5-dioxopyrrolidin-1-yl 3-(2,5-dioxo-2,5-dihydro-1H-pyrrol-1-yl)propanoate (MP-OSu, 1.7 mg, 0.0063 mmol) was added compound 15b (3.9 mg, 0.0042 mmol) as a solution in DMA (423 μL). To the mixture was added DIPEA (3.7 μL, 0.0211 mmol, 5 equiv.) and the reaction mixture was stirred for 30 min at RT, after which glacial acetic acid (3.68 μL) was added, and the product was purified via preparatory HPLC (10-40-95%, 0.05% TFA in acetonitrile, Method G). Fractions containing the desired product were combined and solvents removed via lyophilization to yield compound 15 as trifluoroacetate salt (20% yield, 1.0 mg, 0.0009 mmol). UPLC-MS (Method D, ESI+): m/z [M+H]⁺=931.39 (theoretical); 931.41 (observed). HPLC retention time: 1.62 min.

### Synthesis of S-(1-(3-((3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)(methyl)amino)-3-oxopropyl)-2,5-dioxopyrrolidin-3-yl)-L-cysteine (Compound 16)

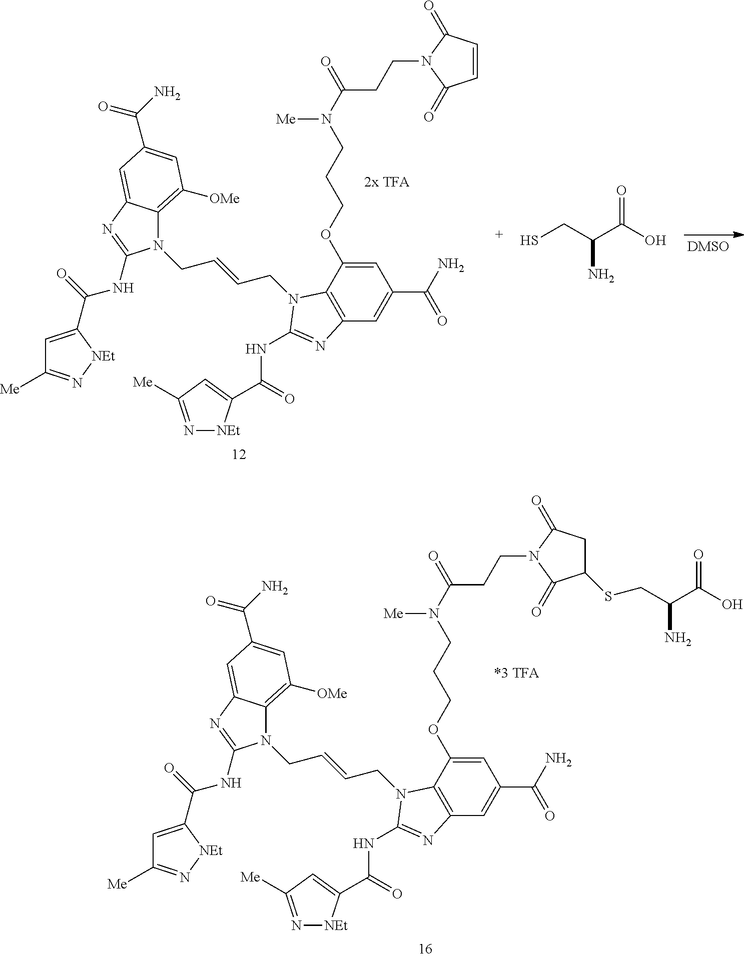

Compound 12 (1.5 mg, 0.0015 mmol, 1 equiv.) was dissolved in DMSO (50 μL). L-cysteine (1 M, 2.2 μL, 0.0022 mmols, 1.5 equiv.) was added as a solution in water. The reaction mixture was stirred at 30° C. for 30 min, and subsequently purified directly via preparatory HPLC (30-70-95%, 0.05% TFA in acetonitrile, Method G). Fractions containing the desired product were combined and frozen. The solvents were removed via lyophilization to yield compound 16 as the trifluoroacetate salt (49% yield, 1.03 mg, 0.0007 mmol). UPLC-MS (Method E, ESI+): m/z [M+H]⁺=1066.42 (theoretical); 1066.44 (observed). HPLC retention time: 1.65 min.

### Synthesis of (S,E)-4-((3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl1H-pyrazole-5-carboxamido)-1H-benzo[d]imidazol-7-yl)oxy)propyl)(methyl)amino)-3-(3-(2,5-dioxo-2,5-dihydro-1H-pyrrol-1-yl)propanamido)-4-oxobutanoic acid (Compound 17)

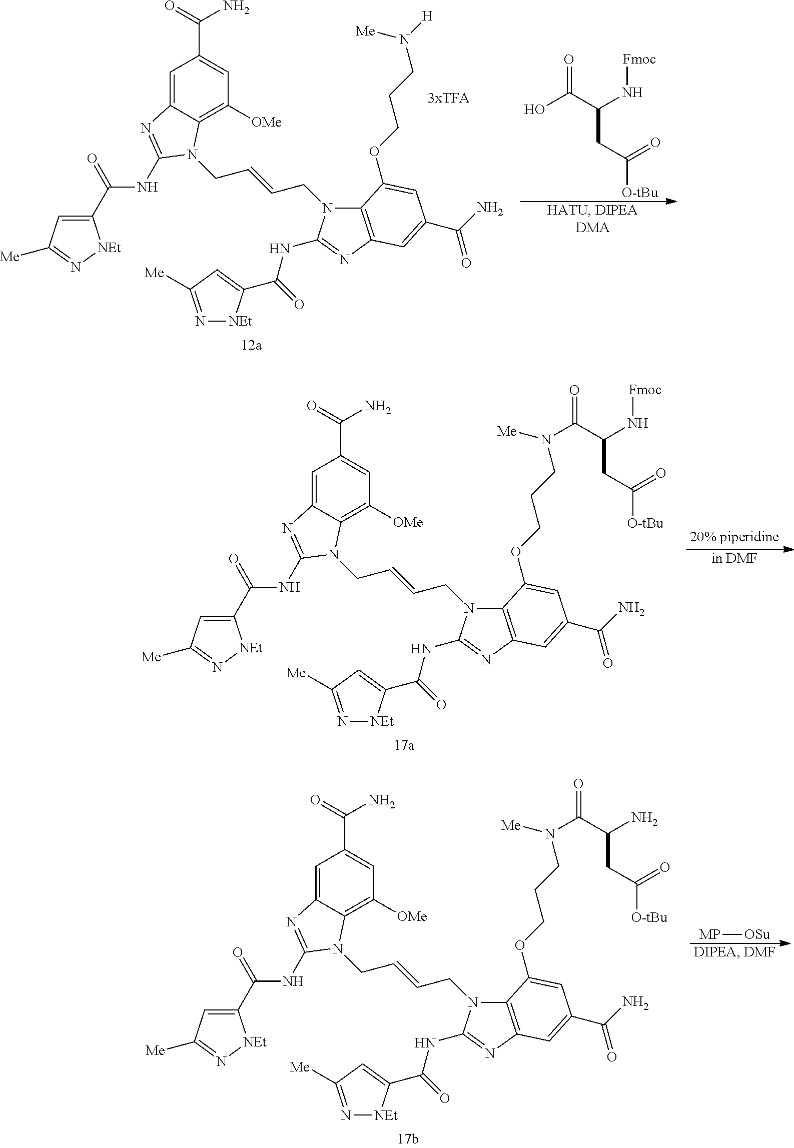

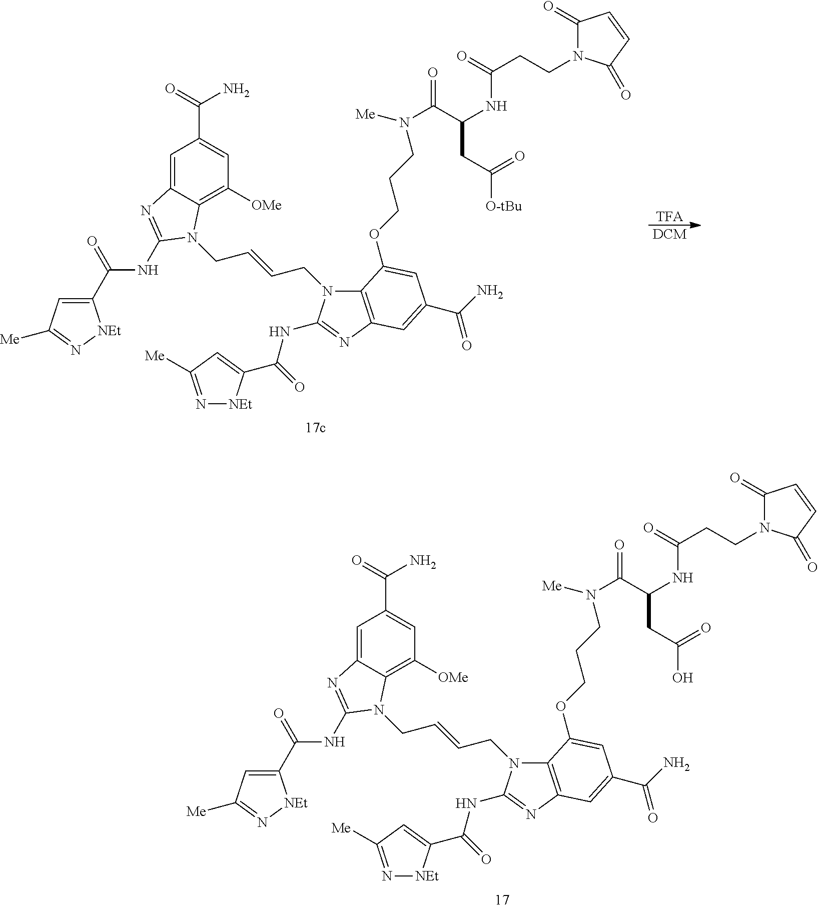

### Synthesis of Compound 17a

An oven dried 4 mL vial equipped with a stir bar was charged with compound 12a (10 mg, 0.011 mmol, 1.0 equiv.), Fmoc-aspartate 4-tert-butyl ester (9.1 mg, 0.022 mmol, 2.0 equiv.) and HATU (8.4 mg, 0.022 mmol, 2.0 equiv.), followed by DMF (0.5 mL) and DIPEA (9.6 uL, 0.055 mmol, 5.0 equiv.). The reaction mixture was stirred at room temperature overnight and full conversion to the amide was observed. Solvent was removed in vacuo, and the resulting crude oil was dissolved in DCM and the desired product isolated by flash chromatography (10 g SiO₂, 0-40% MeOH in DCM) to give 17a (12 mg, 0.0104 mmol, 94% yield) as a light brown solid. The isolated material still contained some impurities, but was used in subsequent steps without further purification. UPLC-MS (Method D, ESI+): m/z [M+H]⁺=1187.54 (theoretical); 1187.53 (observed). HPLC retention time: 2.40 min.

### Synthesis of Compound 17b

An oven dried 4 mL vial equipped with a stir bar was charged with 17a (12 mg, 0.0104 mmol, 1.0 equiv.) and 20% piperidine in DMF (1 mL). The reaction mixture was stirred for 1 hour, solvent removed in vacuo and product purified by prepHPLC (Method G, 5-95% acetonitrile in water) to yield 17b (9.3 mg, 0.0096 mmol, 93% yield). UPLC-MS (Method D, ESI+): m/z [M+H]⁺=965.47 (theoretical); 965.48 (observed). HPLC retention time: 1.68 min.

### Synthesis of Compound 17c

A stock solution of MP-OSu and DIPEA was prepared by dissolving 7.7 mg of MP-OSu and 10 μL of DIPEA in 1.0 mL of DMF. An oven dried 4 mL vial equipped with a stir bar was charged with 17b (9.3 mg, 0.0096 mmol, 1.0 equiv.) and 0.5 mL of the stock solution containing MP-OSu (3.8 mg, 0.014 mmol, 1.5 equiv.) and DIPEA (0.029 mmol, 3 equiv.) was added to the vial. The reaction mixture was stirred at room temperature for 2 hours and solvent removed in vacuo to yield crude 17c, which was used in the next step without any further purification. UPLC-MS (Method D, ESI+): m/z [M+H]⁺=1116.50 (theoretical); 1116.80 (observed). HPLC retention time: 1.51 min.

### Synthesis of Compound 17

A 4 mL vial was charged with compound 17c (10.7 mg, 0.0096 mmol, 1 equiv.) dissolved in 20% (v/v) TFA in DCM (1 mL) and the reaction mixture was stirred at room temperature for 3 hours. Solvent was subsequently removed in vacuo, and the crude product was dissolved in DMSO (0.75 mL) and purified by prepHPLC (Method G, 5-50% MeCN in water) to give Compound 17 (5.4 mg, 0.0051 mmol, 53% yield) as a white solid. UPLC-MS (Method D, ESI+): m/z [M+H]⁺=1060.44 (theoretical); 1061.12 (observed). HPLC retention time: 1.28 min.

### Synthesis of (S,E)-7-(3-(6-amino-2-(3-(2,5-dioxo-2,5-dihydro-1H-pyrrol-1-yl)propanamido)-N-methylhexanamido)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide (Compound 18)

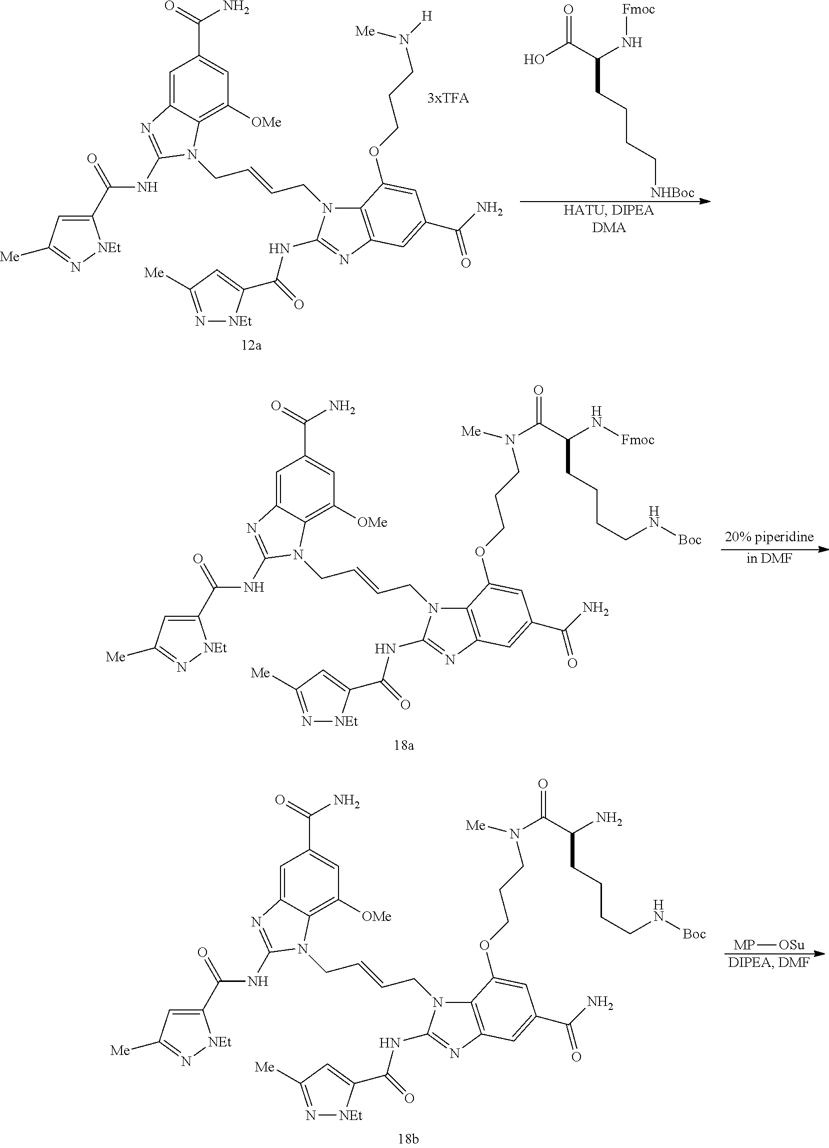

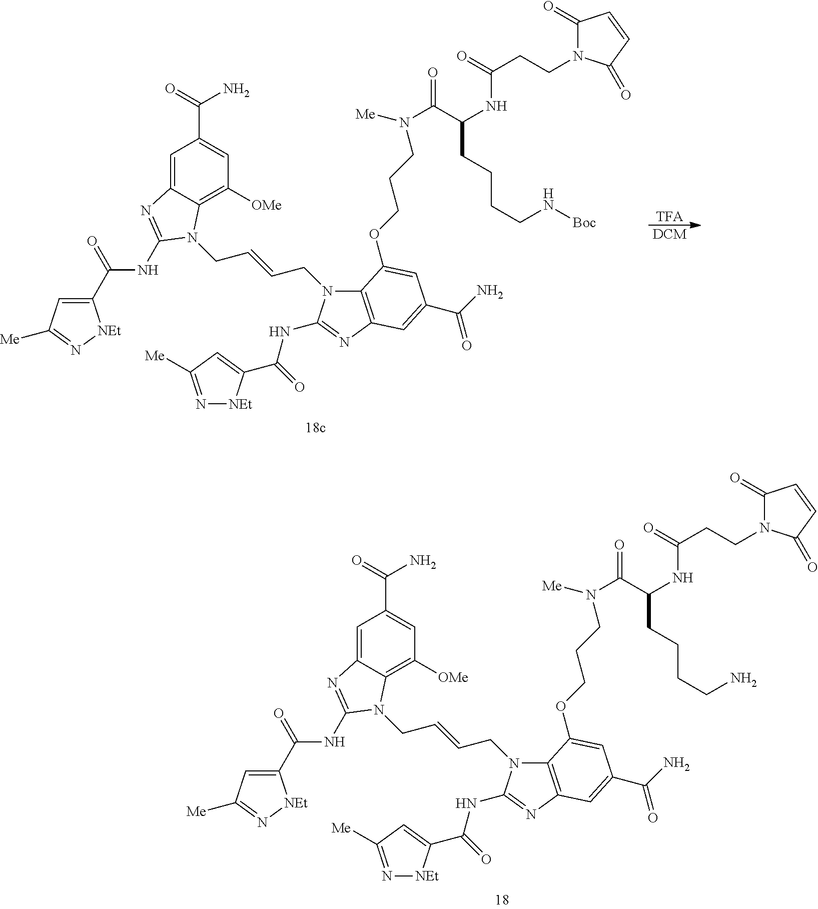

### Synthesis of Compound 18a

An oven dried 4 mL vial equipped with a stir bar was charged with HATU (7.8 mg, 0.021 mmol, 2.0 equiv.) and Fmoc-lysine N-epsilon-Boc (9.6 mg, 0.021 mmol, 2.0 equiv.); to which was added a solution of compound 12a (9.3 mg, 0.0103 mmol, 1.0 equiv.) and DIPEA (9 uL, 0.051 mmol, 5 equiv.) in DMF (0.5 mL). The vial was capped and sealed with parafilm and the mixture was stirred at RT overnight. Full conversion was observed by UPLC-MS (Method D). Solvent was removed in vacuo and product was purified by flash chromatography (10 g SiO₂, 0-40% MeOH in DCM) to give 18a (12 mg, 0.0097 mmol, 95%) as a tan solid. UPLC-MS (Method D, ESI+): m/z [M+H]⁺=1244.60 (theoretical); 1244.61 (observed). HPLC retention time: 2.40 min.

### Synthesis of Compound 18b

An oven-dried 4 mL vial equipped with a stir bar was charged with 18a (12 mg, 0.0096 mmol) and 20% (v/v) piperidine in DMF (1 mL) was added to the reaction. The reaction mixture was stirred until full conversion was observed by UPLC-MS (Method D), which took approximately 1 hour. Solvent was removed in vacuo and product was purified by preparatory HPLC (Method G, 5-95% MeCN in water with 0.1% (v/v) formic acid). The HPLC solvents were removed in vacuo to give 18b (4.2 mg, 0.0041 mmol, 36%) as an off-white solid. UPLC-MS (Method D, ESI+): m/z [M+H]⁺=1022.53 (theoretical); 1022.80 (observed). HPLC retention time: 1.30 min.

### Synthesis of Compound 18e

An oven-dried 4 mL vial equipped with a stir bar was charged with 18b (4.2 mg, 0.0034 mmol, 1 equiv.), followed by MP-OSu (1.8 mg, 0.0068 mmol, 2.0 equiv.), DIPEA (5.9 μL, 0.034 mmol, 10 equiv.) and DMF (0.8 mL). The reaction mixture was stirred at room temperature for 3 hours at which point UPLC-MS (Method D) analysis showed full conversion. Solvent was removed in vacuo to yield the crude product 18c, which was used in the next step without purification. UPLC-MS (Method D, ESI+): m/z [M+H]⁺=1173.56 (theoretical); 1173.94 (observed). HPLC retention time: 1.54 min.

### Synthesis of Compound 18

An oven-dried 4 mL vial containing a stir bar was charged with crude 18c from the previous step (0.0034 mmol) and 20% (v/v) TFA in DCM (1 mL) was added. The reaction mixture was stirred for one hour and the product was subsequently purified by preparatory HPLC (Method G, 5-50% MeCN in water with 0.1% (v/v) formic acid). The HPLC solvents were removed in vacuo to give 18c (4.2 mg, 0.0035 mmol, 56% yield over 2 steps) as a white solid. UPLC-MS (Method D, ESI+): m/z [M+H]⁺=1073.51 (theoretical); 1073.73 (observed). HPLC retention time: 1.15 min.

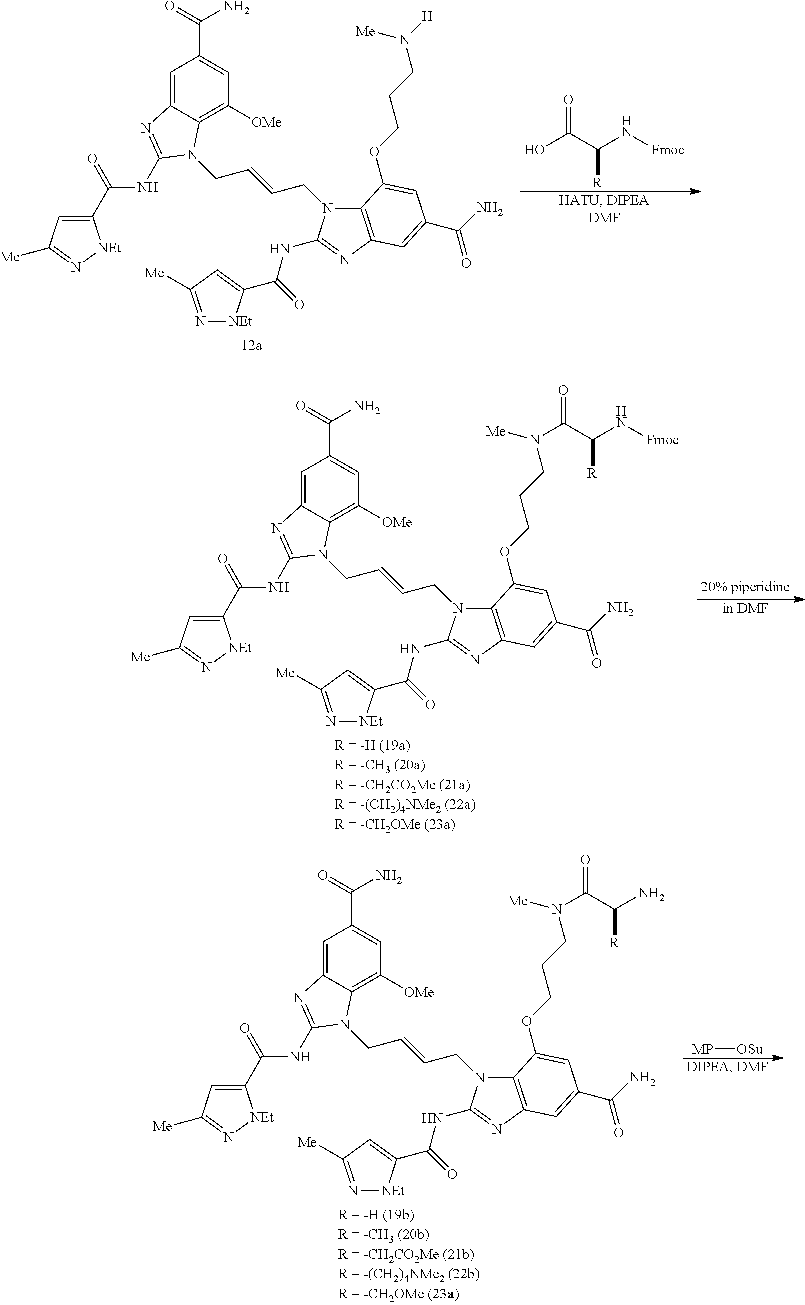

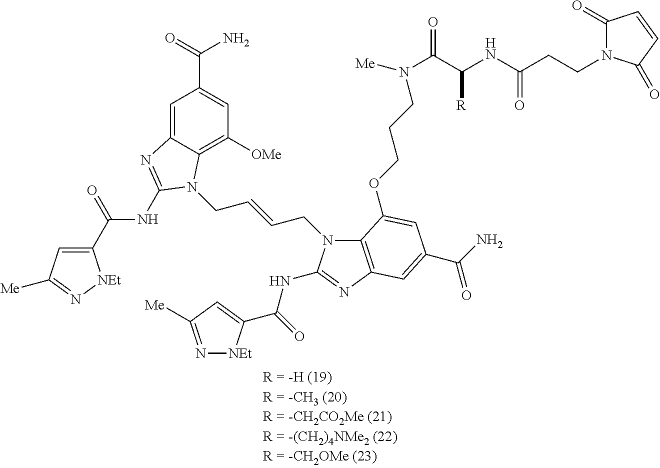

### General Methods for HATU Coupling, Fmoc Deprotection, and MP Coupling

HATU coupling (Method 1): An oven-dried 4 mL vial equipped with a stir bar was charged with compound 12a (1.0 equiv.), HATU (2.0 equiv.), DIPEA (5 equiv.) and DMF (20 mM in 12a) and the reaction mixture was stirred at room temperature overnight. The solvent was removed in vacuo and product purified via chromatography.

Fmoc deprotection (Method 2): An oven-dried 4 mL vial equipped with a stir bar was charged with the HATU coupled product from above, which was dissolved in 20% (v/v) piperidine in DMF (1 mL). The reaction mixture was stirred at room temperature for 1 hour, solvent removed in vacuo, and product purified via chromatography.

MP coupling (Method 3): An oven-dried 4 mL vial equipped with a stir bar was charged with the product from the previous reaction, to which was added MP-OSu (2 equiv.) and DIPEA (10 equiv.) and DMF (10 mM in Fmoc-deprotected amine starting material). The reaction mixture was stirred at room temperature for 3 hours, solvent removed in vacuo and product purified by preparatory HPLC.

Compound 19a was prepared according to General Method 1 (8.0 mg, 0.0075 mol, 85% yield). UPLC-MS (Method D, ESI+): m/z [M+H]⁺=1073.47 (theoretical); 1074.03 (observed). HPLC retention time: 1.76 min.

Compound 19b was prepared according to General Method 2 (6.1 mg, 0.0072 mol, 95% yield). UPLC-MS (Method D, ESI+): m/z [M+H]⁺=851.41 (theoretical); 851.69 (observed). HPLC retention time: 1.15 min.

Compound 19 was prepared according to General Method 3 (4.3 mg, 0.0043 mol, 60% yield). UPLC-MS (Method D, ESI+): m/z [M+H]⁺=1002.43 (theoretical); 1002.72 (observed). HPLC retention time: 1.31 min.

Compound 20a was prepared according to General Method 1 (8.7 mg, 0.0080 mol, 91% yield). UPLC-MS (Method D, ESI+): m/z [M+H]⁺=1087.49 (theoretical); 1087.90 (observed). HPLC retention time: 1.75 min.

Compound 20b was prepared according to General Method 2 (5.6 mg, 0.0065 mol, 81% yield). UPLC-MS (Method D, ESI+): m/z [M+H]⁺=865.42 (theoretical); 865.66 (observed). HPLC retention time: 1.12 min.

Compound 20 was prepared according to General Method 3 (3.4 mg, 0.0034 mol, 52% yield). UPLC-MS (Method D, ESI+): m/z [M+H]⁺=1016.45 (theoretical); 1017.08 (observed). HPLC retention time: 1.33 min.

Compound 21a was prepared according to General Method 1 (14 mg, 0.0119, mmol). UPLC-MS (Method D, ESI+): m/z [M+H]⁺=1145.50 (theoretical); 1145.42 (observed). HPLC retention time: 1.74 min.

Compound 21b was prepared according to General Method 2 (7.2 mg, 0.0078 mol, 76% yield over 2 steps). UPLC-MS (Method D, ESI+): m/z [M+H]⁺=923.43 (theoretical); 923.67 (observed). HPLC retention time: 1.13 min.

Compound 21 was prepared according to General Method 3 (1.5 mg, 0.0014 mols, 22% yield). UPLC-MS (Method D, ESI+): m/z [M+H]⁺=1074.45 (theoretical); 1074.90 (observed). HPLC retention time: 1.36 min.

Compound 22a was prepared according to General Method 1 (7.6 mg, 0.0065 mols, 63% yield). UPLC-MS (Method D, ESI+): m/z [M+H]⁺=1172.58 (theoretical); 1172.59 (observed). HPLC retention time: 1.84 min.

Compound 22b was prepared according to General Method 2 (6.1 mg, 0.0064 mmols, 57% yield). UPLC-MS (Method D, ESI+): m/z [M+H]⁺=950.51 (theoretical); 950.83 (observed). HPLC retention time: 0.99 min.

Compound 22 was prepared according to General Method 1 (2.6 mg, 0.0023 mols, 37% yield). UPLC-MS (Method D, ESI+): m/z [M+H]⁺=1101.54 (theoretical); 1101.96 (observed). HPLC retention time: 1.18 min.

Compound 23a was prepared according to General Method 1 (12 mg, 0.0105 mmol). UPLC-MS (Method D, ESI+): m/z [M+H]⁺=1117.50 (theoretical); 1117.77 (observed). HPLC retention time: 1.75 min.

Compound 23b was prepared according to General Method 2 (7.2 mg, 0.00804 mmol, 91% over 2 steps). UPLC-MS (Method D, ESI+): m/z [M+H]⁺=895.43 (theoretical); 895.73 (observed). HPLC retention time: 1.12 min.

Compound 23 was prepared according to General Method 3 (8.4 mg, 0.0047, 58% yield). UPLC-MS (Method D, ESI+): m/z [M+H]⁺=1046.46 (theoretical); 1047.06 (observed). HPLC retention time: 1.36 min.

### Synthesis of (S,E)-1-(4-(5-carbamoyl-2-(1-ethyl-3-methyl-1H-pyrazole-5-carboxamido)-7-methoxy-1H-benzo[d]imidazol-1-yl)but-2-en-1-yl)-7-(3-(2-(3-(2,5-dioxo-2,5-dihydro-1H-pyrrol-1-yl)propanamido)-3-hydroxy-N-methylpropanamido)propoxy)-2-(1-ethyl-3-methyl-1H-pyrazole-5-carboxamido)-1H-benzo[d]imidazole-5-carboxamide (Compound 24)

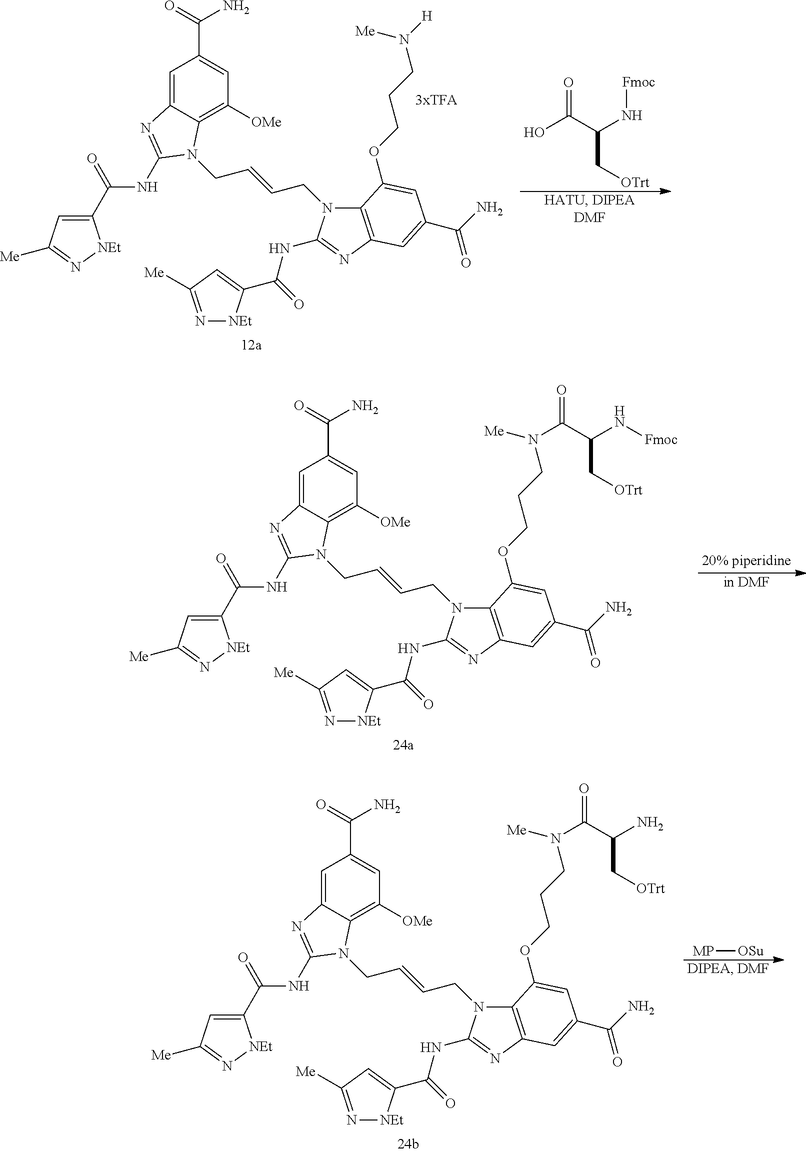

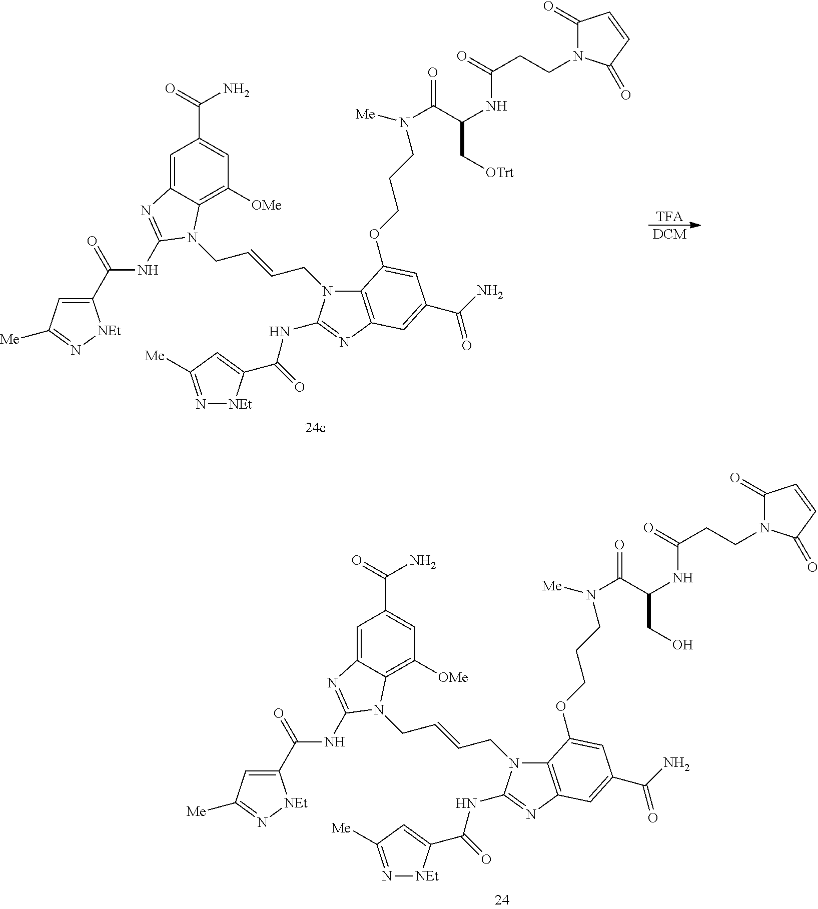

### Synthesis of Compound 24a

An oven dried 4 mL vial equipped with a stir bar was charged with HATU (6.7 mg, 0.018 mmol, 2.0 equiv.) and 2-(9H-fluoren-9-ylmethoxycarbonylamino)-3-methoxy-propanoic acid (6.0 mg, 0.018 mmol, 2.0 equiv.), and a solution of compound 12a (8 mg, 0.0088 mmols, 1.0 equiv.) and DIPEA (8 uL, 0.044 mmols, 5 equiv.) in DMF (0.5 mL) was added to the vial. The vial was capped and sealed with parafilm and the reaction mixture was stirred at room temperature overnight, upon which full conversion was observed by UPLC-MS (Method D). Solvent was removed in vacuo and product purified by flash chromatography (10 g SiO₂, 0-40% MeOH in DCM) to give 24a (15 mg), which was used in the next reaction without further purification. UPLC-MS (Method D, ESI+): m/z [M+H]⁺=1345.59 (theoretical); 1346.12 (observed). HPLC retention time: 2.23 min.

### Synthesis of Compound 24b

An oven-dried 4 mL vial equipped with a stir bar was charged with 24a (15 mg, 0.011 mmol) and 20% (v/v) piperidine in DMF (1 mL) was added to it. The reaction mixture was stirred until full conversion was observed by UPLC-MS (Method D), which took approximately 1 hour. Solvent was removed in vacuo and the crude product was purified by preparatory HPLC (Method G, 5-95% MeCN in water with 0.1% (v/v) formic acid); the HPLC solvents were removed in vacuo to give 24b (8.4 mg, 0.0075 mmol, 94% over 2 steps) as an off-white solid. UPLC-MS (Method D, ESI+): m/z [M+H]⁺=1123.53 (theoretical); 1123.98 (observed). HPLC retention time: 1.47 min.

### Synthesis of Compound 24c

An oven-dried 4 mL vial equipped with a stir bar was charged with 24b (8.4 mg, 0.0075 mmol, 1 equiv.), followed by MP-OSu (3.0 mg, 0.011 mmol, 1.5 equiv.), DIPEA (3.9 μL, 0.022 mmol, 3 equiv.) and DMF (0.5 mL). The reaction mixture was stirred at room temperature for 3 hours at which point UPLC-MS (Method D) analysis showed full conversion. Solvent was removed in vacuo and the resulting crude product was used in the next step without purification. UPLC-MS (Method D, ESI+): m/z [M+H]⁺=1274.55 (theoretical); 1275.21 (observed). HPLC retention time: 1.89 min.

### Synthesis of Compound 24

An oven-dried 4 mL vial containing a stir bar was charged with crude 24c (0.0075 mmol) and 20% (v/v) TFA in DCM (1 mL) was added to the vial. The reaction mixture was stirred for 20 minutes, and solvent removed in vacuo. The resulting crude product was dissolved in DMSO (0.5 mL) and purified by preparatory HPLC (Method G, 5-50% MeCN in water with 0.1% (v/v) formic acid) and solvent removed in vacuo to give 24 (4.0 mg, 0.0031 mmol, 42% yield over 2 steps) as a white solid. UPLC-MS (Method D, ESI+): m/z [M+H]⁺=1032.44 (theoretical); 1033.09 (observed). HPLC retention time: 1.28 min.

### Synthesis of (E)-7-(3-(3-(2,5-dioxo-2,5-dihydro-1H-pyrrol-1-yl)-N-methylpropanamido)propoxy)-2-(1-ethyl-3-methyl-1H-pyrazole-5-carboxamido)-1-(4-(2-(1-ethyl-3-methyl-1H-pyrazole-5-carboxamido)-5-sulfamoyl-1H-benzo[d]imidazol-1-yl)but-2-en-1-yl)-1H-benzo[d]imidazole-5-carboxamide (Compound 25)

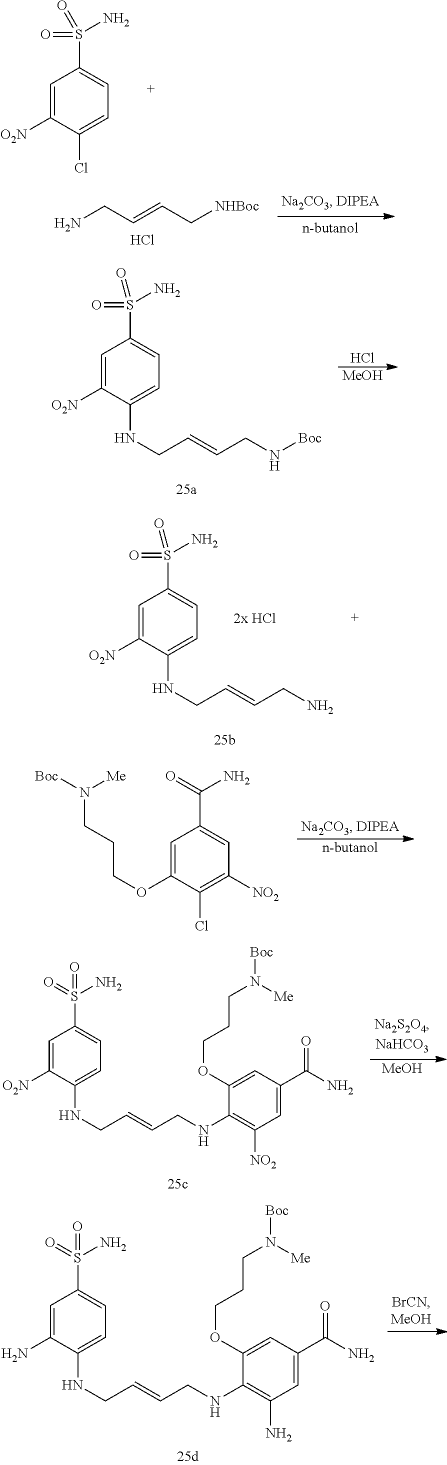

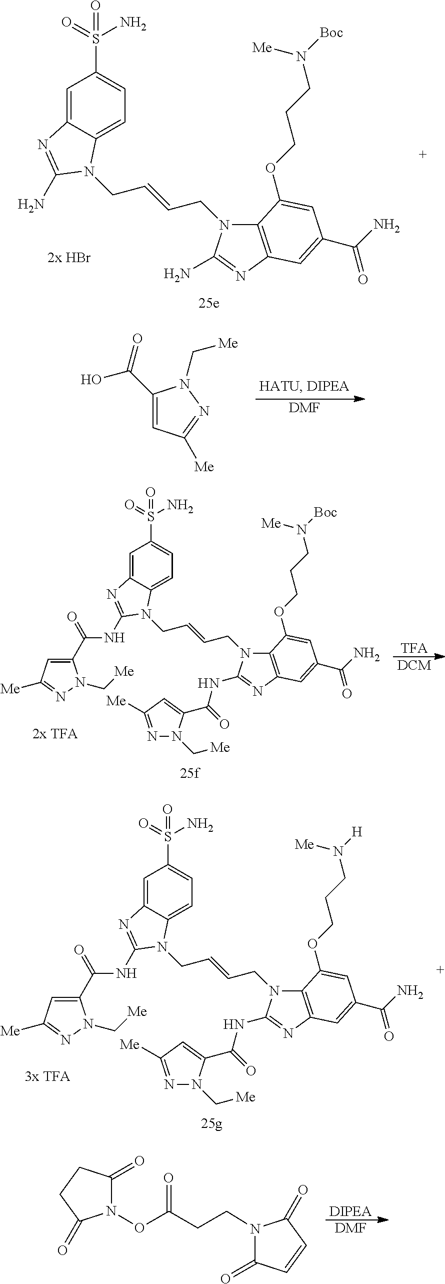

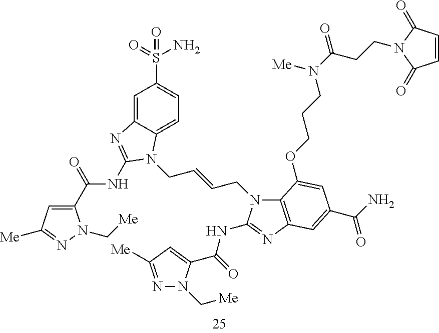 [SMILES: CCn1nc(C)cc1C(=O)Nc1nc2cc(S(N)(=O)=O)ccc2n1C/C=C/Cn1c(NC(=O)c2cc(C)nn2CC)nc2cc(C(N)=O)cc(OCCCN(C)C(=O)CCN3C(=O)C=CC3=O)c21]

### Synthesis of 25a

A 5 mL oven-dried microwave vial with stir bar was charged with 4-chloro-3-nitro-benzenesulfonamide (250 mg, 1.06 mmol, 1 equiv.), tert-butyl N-[(E)-4-aminobut-2-enyl]carbamate hydrochloride (353 mg, 1.6 mmol, 1.5 equiv.) and sodium carbonate (336 mg, 3.2 mmol, 3 equiv.). To the vial was added 1-butanol (3 mL) followed by DIPEA (1.1 mL, 6.34 mmol, 6 equiv.) and additional 1-butanol to bring the total volume of the reaction up to 5 mL. The vial was sealed and heated to 140° C. in the microwave reactor for 120 minutes.

The crude product was poured into brine (100 mL) and extracted with EtOAc (3×200 mL), organics combined, washed with brine (2×100 mL), dried with MgSO₄, filtered and solvent removed in vacuo to give a bright red oil. This material was purified by flash chromatography (dry loaded on celite, 25g Sfar, HC Duo, SiO₂ column, 0-40% MeOH in DCM) to give 25a (295 mg, 0.763 mmol, 72% yield) as a bright yellow solid. UPLC-MS (Method D, ESI+): m/z [M+H−Boc]⁺=287.1 (theoretical); 287.4 (observed). HPLC retention time: 1.53 min.

### Synthesis of 25b

A 20 mL vial was charged with 25a (295 mg, 0.763 mmol, 1 equiv.) which was dissolved in methanol (7.5 mL) and 4M HCl in 1,4-dioxane (40 eq, 7.5 mL, 30.0 mmol). The solution was stirred at 40° C. for 30 minutes and solvent removed in vacuo to give 25b as the 2× HCl salt (274 mg, 0.764 mmol, quantitative yield) as a bright red solid. UPLC-MS (Method D, ESI+): m/z [M+H]⁺=287.1 (theoretical); 287.6 (observed). HPLC retention time: 0.52 min.

### Synthesis of 25c

An oven dried 5 mL microwave vial with stir bar was charged with 25b (135 mg, 0.376 mmol, 1 equiv.), tert-butyl N-[3-(5-carbamoyl-2-chloro-3-nitro-phenoxy)propyl]carbamate (211 mg, 0.564 mmol, 1.5 equiv., prepared as described below) and sodium carbonate (119 mg, 1.13 mmol, 3 equiv.) which was followed by addition of n-butanol (3.75 mL) and DIPEA (0.39 mL, 2.25 mmol, 6 equiv.). The vial was sealed and heated to 140° C. for 3 hours in a microwave reactor to give a bright red heterogenous mixture. This solution was filtered over celite washing with 1:1 DCM:MeOH (100 mL), solvent removed in vacuo and crude product was loaded onto celite and purified by flash chromatography (25 g SiO₂ column, 0-40% MeOH in DCM) to give 25c (245 mg, 0.384 mmol) as a mixture of product and starting material (3:2). Product mixture was used in the next step without any further purification. UPLC-MS (Method D, ESI+): m/z [M+H]⁺=638.2 (theoretical); 638.5 (observed). HPLC retention time: 1.75 min.

### Synthesis of 25d

A 20 mL vial with stir bar was charged with 25c (245 mg, 0.384 mmol, 1 equiv.) and sodium bicarbonate (580 mg, 6.90 mmol, 18 equiv.) and methanol (4 mL) was added. To the vial was then added sodium hydrosulfite (1.20 g, 6.90 mmol, 18 equiv. in 4 mL water) and the vial was heated to 50° C. for 60 minutes. The reaction was cooled to room temperature, filtered over celite washing with MeOH (50 mL) and DCM (50 mL) and the crude product loaded onto celite. The product was purified by flash chromatography (25g Sfar HC Duo, SiO₂ column, 0-40% 10:1 MeOH:NH₄OH in DCM) to give 25d (89 mg, 0.154 mmol, 41% yield over 2 steps) as a mixture of inseparable rotational conformers. UPLC-MS (Method D, ESI+): m/z [M+H]⁺=578.3 (theoretical); 578.5 (observed). HPLC retention time: 0.98 & 1.18 min.

### Synthesis of 25e

Two identical reactions were setup side by side. An oven dried 4 mL vial with stir bar was charged with 25d (45 mg, 0.156 mmol, 1 equiv.), dissolved in methanol (1 mL) and cyanogen bromide (200 uL, 1.20 mmol, 8 equiv.) was added. Reaction was stirred overnight, and solvent removed in vacuo and two reactions combined to give 25e as the 2× HBr salt (120 mg, 0.15 mmol, 97% yield) as a light gray solid. UPLC-MS (Method D, ESI+): m/z [M+H]⁺=628.3 (theoretical); 628.4 (observed). HPLC retention time: 0.79 min.

### Synthesis of 25f

An oven dried 4 mL vial with stir bar was charged with 25e (120 mg, 0.152 mmol, 1 equiv.), 2-ethyl-5-methyl-pyrazole-3-carboxylic acid (94 mg, 0.61 mmol, 4.0 equiv.) and HATU (231 mg, 0.61 mmol, 4 equiv.). The solids were dissolved in DMF (1 mL) and DIPEA (0.22 mL, 1.2 mmol, 8 equiv.) was added. The reaction was stirred at room temperature overnight, acetic acid was added (100 uL) and product purified by prepHPLC (Method I, 5-95% MeCN in water with 0.05% TFA) and solvent removed in vacuo to give 25f (107 mg, 0.12 mmol, 78% yield) as an off-white solid. UPLC-MS (Method D, ESI+): m/z [M+H]⁺=900.4 (theoretical); 900.6 (observed). HPLC retention time: 1.69 min.

### Synthesis of 25g

Compound 25f (107 mg, 0.12 mmol, 1 equiv.) was added to a 20 mL vial with stir bar and dissolved in 20% TFA in DCM (5 mL). Reaction was stirred at room temperature for 20 minutes and then solvent removed in vacuo to give 25g as the 3×TFA salt and an off-white solid (70 mg, 0.0615 mmol, 52% yield). A sample of analytical purity was obtained by prepHPLC purification (Method G, 5-95% MeCN in water with 0.05% TFA). UPLC-MS (Method D, ESI+): m/z [M+H]⁺=800.3 (theoretical); 800.6 (observed). HPLC retention time: 1.12 min.

### Synthesis of 25

An oven dried 4 mL vial with stir bar was charged with 25g (12 mg, 0.011 mmol, 1 equiv.) which was dissolved in DMF (1 mL) and then both DIPEA (15 uL, 0.087 mmol, 8 equiv.) and MP-OSu (4.3 mg, 0.0163 mmol, 1.5 equiv.) were added to the reaction. The solution was stirred at room temperature for 30 minutes, quenched with 20% TFA in DCM (100 uL) and purified by prepHPLC (Method G, 5-95% MeCN in water with 0.05% TFA) to 25 as the 2×TFA salt (5.7 mg, 0.0048 mmol, 45% yield). UPLC-MS (Method D, ESI+): m/z [M+H]⁺=951.4 (theoretical); 951.2 (observed). HPLC retention time: 2.18 min.

### Synthesis of (E)-1-(4-(5-carbamoyl-2-(1-ethyl-3-methyl-1H-pyrazole-5-carboxamido)-7-methoxy-1H-benzo[d]imidazol-1-yl)but-2-en-1-yl)-7-(2-(1-(3-(2,5-dioxo-2,5-dihydro-1H-pyrrol-1-yl)propanoyl)azetidin-3-yl)ethoxy)-2-(1-ethyl-3-methyl-1H-pyrazole-5-carboxamido)-1H-benzo[d]imidazole-5-carboxamide (Compound 26)

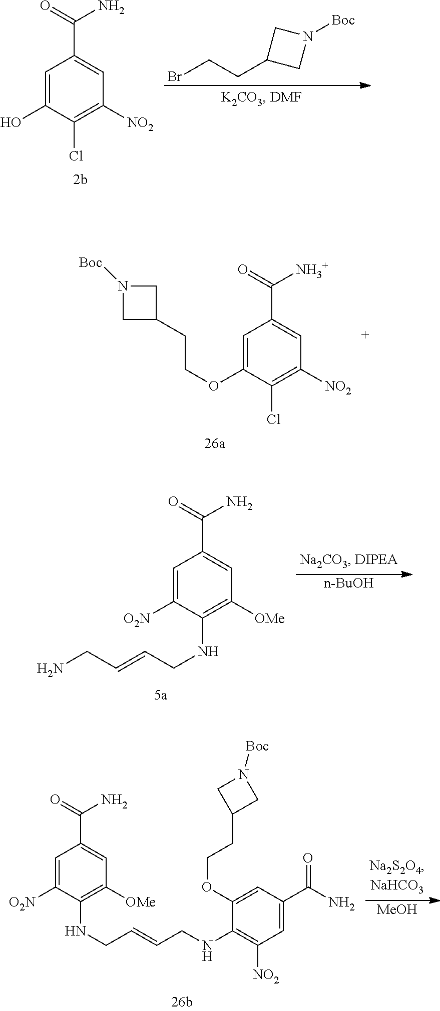

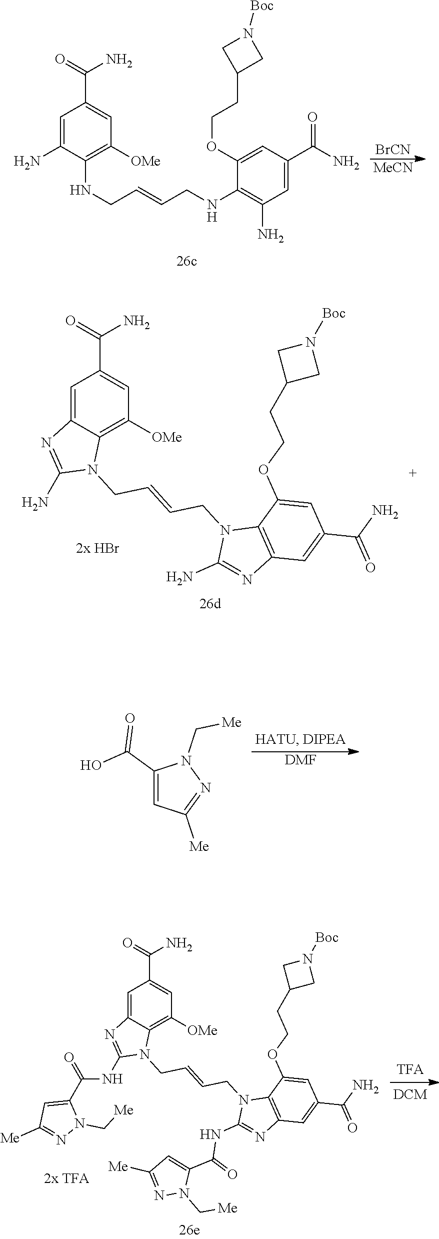

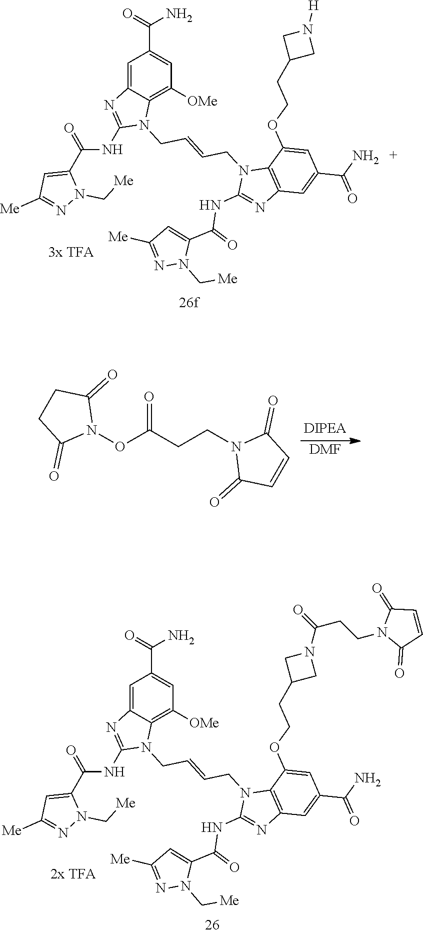

### Synthesis of 26a

An oven dried 8 mL Vial with stir bar was charged with 2b (100 mg, 0.462 mmol, 1 equiv.) and potassium carbonate (191 mg, 1.39 mmol, 3 equiv.) followed by addition of tert-butyl 3-(2-bromoethyl)azetidine-1-carboxylate (152 mg, 0.577 mmol, 1.25 equiv.). The starting materials were dissolved in DMF (3 mL), vial sealed with parafilm and stirred at 70° C. for 24 hours. The crude material was poured into a separatory funnel containing saturated ammonium chloride (100 mL) and EtOAc (100 mL each), shaken, layers separated, and aqueous layer extracted with EtOAc (2×100 mL). The combined organic fractions were washed with brine (2×50 mL), dried with MgSO₄, filtered and solvent removed in vacuo to give crude product as a light-yellow solid. The crude product was purified by flash chromatography (25g Sfar HC Duo SiO₂ column, 0-20% MeOH in DCM) to give 26a as a yellow solid (86 mg, 0.215 mmol, 47% yield). UPLC-MS (Method D, ESI+): m/z [M+H]⁺=400.1 (theoretical); 400.5 (observed). HPLC retention time: 1.79 mi.

### Synthesis of 26b

An oven-dried 2 mL microwave vial was charged with 25a (35 mg, 0.0875 mmol, 1 equiv.), 5a (62 mg, 0.175 mmol, 2 equiv.) and sodium carbonate (28 mg, 0.263 mmol, 3 equiv.) and to this vial was added n-butanol (1 mL) and DIPEA (0.1 mL, 0.5 mmol, 6 equiv.). The vial was sealed and heated to 140° C. for 3 hours in a microwave reactor. The reaction was then filtered over celite washing with 1:1 MeOH:DCM (100 mL), solvent removed in vacuo and crude material loaded onto celite. The product was purified by flash chromatography (25g Sfar HC Duo SiO₂ column, 0-20% MeOH in DCM) to give 25b as a bright red solid (38 mg, 0.0592 mmol, 68% yield). UPLC-MS (Method D, ESI+): m/z [M+H]⁺=644.3 (theoretical); 644.6 (observed). HPLC retention time: 1.72 min.

### Synthesis of 26c

An oven-dried 4 mL vial was charged with 25b (38 mg, 0.0592 mmol, 1 equiv.) which was dissolved in methanol (1 mL) and sodium bicarbonate (90 mg, 1.1 mmol, 18 equiv.) was added followed by sodium hydrosulfite (186 mg, 1.07 mmol, 18 equiv.) as a solution in water (1 mL). The reaction was heated to 50° C. for 1 hour and filtered over celite washing with 1:1 DCM:MeOH (50 mL). The crude product was loaded onto celite and purified by flash chromatography (25g Sfar HC Duo, SiO₂ column, 0-40% 10:1 MeOH:NH₄OH in DCM) to give 25c (10 mg, 0.017 mmol, 29% yield) as a light yellow solid. UPLC-MS (Method D, ESI+): m/z [M+H]⁺=584.3 (theoretical); 584.6 (observed). HPLC retention time: 1.18 min.

### Synthesis of 26d

An oven dried 4 mL vial with stir bar was charged with 25c (10 mg, 0.017 mmol, 10 equiv.) which was dissolved in methanol (0.5 mL) and cyanogen bromide (0.050 mL, 0.150 mmol, 3M in DCM, 8.7 equiv.) was added. The reaction was stirred for 18 hours and solvent removed in vacuo to give the 25d as a light grey solid and the 2× HBr salt (13 mg, 0.0165 mmol, 95% yield) which was used without any further purification. UPLC-MS (Method D, ESI+): m/z [M+H]⁺=634.3 (theoretical); 634.6 (observed). HPLC retention time: 0.98 min.

### Synthesis of 26e

An oven dried 4 mL vial with stir bar was charged with 25d (13 mg, 0.0165 mmol, 1 equiv.), HATU (25 mg, 0.066 mmol, 4 equiv.) and 2-ethyl-5-methyl-pyrazole-3-carboxylic acid (10 mg, 0.066 mmol, 4 equiv.) which were dissolved in DMF (0.5 mL) and then DIPEA (0.050 mL, 0.20 mmol, 17 equiv.) was added. The reaction was stirred at room temperature for 24 hours. The reaction was quenched with acetic acid (100 uL) and product purified by via prepHPLC (Method H., 5-95% MeCN in water with 0.05% TFA) to give 25e as the 2×TFA salt (14 mg, 0.016 mmol, 95% yield) as a light tan solid. UPLC-MS (Method D, ESI+): m/z [M+H]⁺=906.4 (theoretical); 906.3 (observed). HPLC retention time: 2.44 min.

### Synthesis of 26f

An oven dried 4 mL vial with stir bar was charged with 25e (14 mg, 0.016 mmol, 1 equiv.) which was dissolved in 20% TFA in DCM (1 mL) and stirred at room temperature for 15 minutes. Solvent was removed in vacuo to give 25f as the 3×TFA salt (15 mg, 0.013 mmol, 82% yield) as a white solid and the product used without any further purification. UPLC-MS (Method D, ESI+): m/z [M+H]⁺=806.4 (theoretical); 806.6 (observed). HPLC retention time: 1.25 min.

### Synthesis of 26

An oven dried 4 mL vial with stir bar was charged with 25f (5.7 mg, 0.0050 mmol, 1 equiv.) in DMSO (0.5 mL) and MP-OSu (2.0 mg, 0.00750 mmol, 1.5 equiv.) and DIPEA (5 uL, 0.030 mmol, 6 equiv.) was added. The reaction was stirred at room temperature for 1 hour. The reaction was quenched added 20% TFA in DCM (100 uL) and product purified by prepHPLC (Method G, 5-95% MeCN in water with 0.05% TFA) to give 25 as the 2×TFA salt (3.8 mg, 0.00321 mmol, 64% yield) as a white solid. UPLC-MS (Method D, ESI+): m/z [M+H]⁺=957.4 (theoretical); 957.3 (observed). HPLC retention time: 2.19 min.

### Synthesis of (E)-1-(4-(5-carbamoyl-2-(1-ethyl-3-methyl-1H-pyrazole-5-carboxamido)-7-methoxy-1H-benzo[d]imidazol-1-yl)but-2-en-1-yl)-7-(3-((1-(3-(2,5-dioxo-2,5-dihydro-1H-pyrrol-1-yl)propanoyl)azetidin-3-yl)oxy)propoxy)-2-(1-ethyl-3-methyl-1H-pyrazole-5-carboxamido)-1H-benzo[d]imidazole-5-carboxamide (Compound 27)

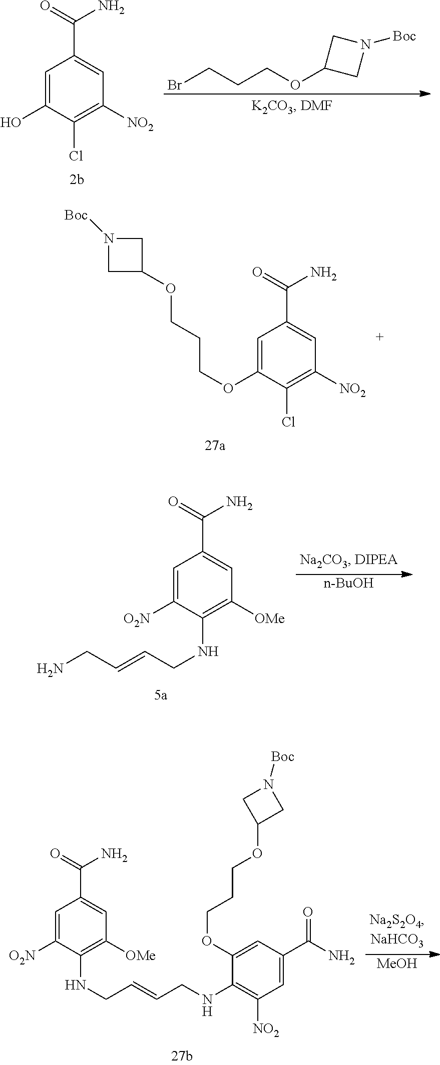

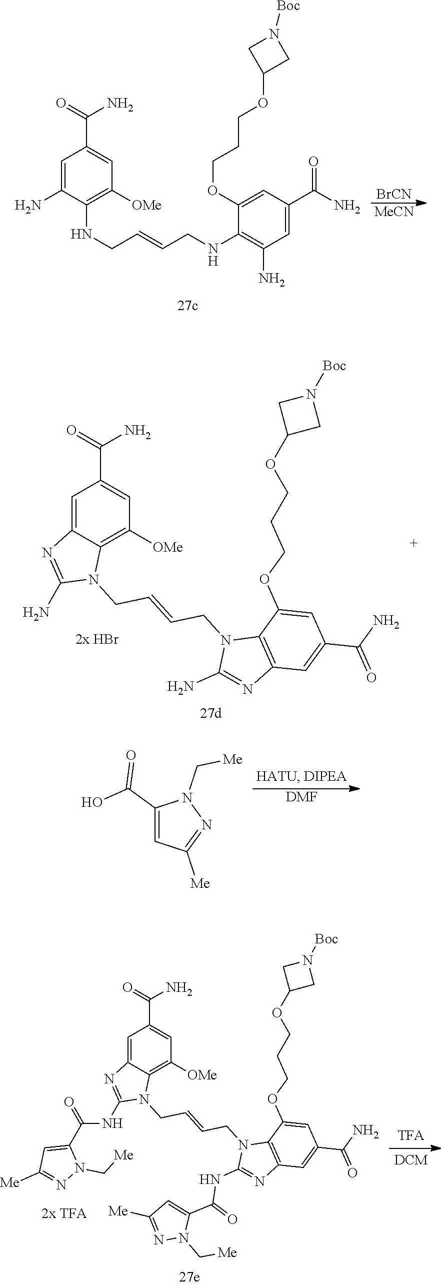

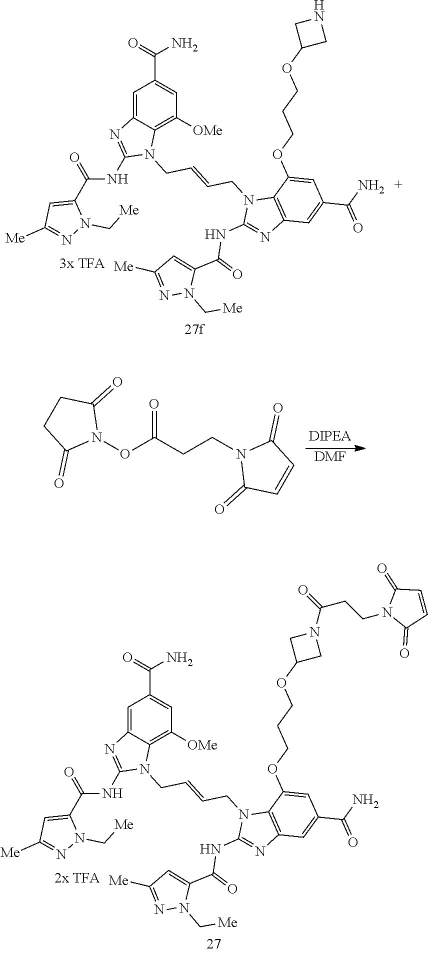

### Synthesis of 27a

An oven dried 8 mL vial with stir bar was charged with 2b as the TFA salt (150 mg, 0.454 mmol, 1 equiv.), tert-butyl 3-(3-bromopropoxy)azetidine-1-carboxylate (133 mg, 0.454 mmol, 1 equiv.) and potassium carbonate (141 mg, 1.02 mmol, 2.3 equiv.) which were dissolved in DMF (4.5 mL) and heated to 55° C. for 24 hours. The reaction was poured into a separatory funnel containing sat. NaHCO₃ (100 mL) and EtOAc (100 mL), shaken, layers separated, and aqueous layer extracted with EtOAc (3×50 mL). The organic fractions were combined and further washed with sat. NaHCO₃ (3×50 mL) and brine (2×50 mL). They were then dried with MgSO₄, filtered and solvent removed in vacuo to 27a (194 mg, 0.353 mmol, 78% yield) as a light yellow solid in a 4:1 ratio of starting material to product and used without further purification. MS (Method D, ESI+): m/z [M+H]⁺=430.1 (theoretical); 430.6 (observed). HPLC retention time: 1.82 min.

### Synthesis of 27b

An oven-dried 5 mL microwave vial was charged with Sodium carbonate (144 mg, 1.36 mmol, 3.00 eq), 5a as the 2×HCL salt (240 mg, 0.678 mmol, 1.50 eq) and 27a (194 mg, 0.452 mmol, 1 equiv.) and then 1-butanol (4 mL) and DIPEA (0.5 mL, 2.7 mmol, 6 equiv.) were added. The vial was sealed and heated to 140° C. for 3 hours in a microwave reactor. The reaction was cooled to room temperature and solution was filtered over celite washing with 1:1 MeOH:DCM (100 mL). The crude product was loaded onto celite and purified by flash chromatography (25g Sfar HC Duo, SiO₂ column, 0-20% MeOH in DCM) to give 27b (95 mg, 0.141 mmol, 31% yield) as a bright red solid. MS (Method D, ESI+): m/z [M+H]⁺=674.3 (theoretical); 674.6 (observed). HPLC retention time: 1.73 min.

### Synthesis of 27c

A 20 mL vial was charged 27b (95 mg, 0.141 mmol, 1 equiv.) and sodium bicarbonate (442 mg, 5.3 mmol, 37 equiv.) and starting material dissolved in methanol (4 mL). To the vial was added sodium hydrosulfite (442 mg, 2.54 mmol, 18 equiv.) as solution in water (4 mL) and reaction was heated, open to the atmosphere, to 50° C. for 1 hour. The solution went from bright red to light yellow over the course of an hour. The reaction was filtered, filter cake washed with 1:1 MeOH:DCM (3×50 mL), solvent removed in vacuo, crude product redissolved in 1:1 MeOH:DCM (100 mL) and filtered over celite. The crude product was loaded onto celite and purified by flash chromatography (25g Sfar HC Duo, SiO₂ column, 0-40% 10:1 MeOH:NH₄OH in DCM) to give 27c (42 mg, 0.0689 mmol, 49% yield) as an off-white solid. UPLC-MS (Method D, ESI+): m/z [M+H]⁺=614.3 (theoretical); 614.5 (observed). HPLC retention time: 0.78 min.

### Synthesis of 27d

An oven-dried 4 mL vial was charged with 27c (42 mg, 0.0689 mmol, 1 equiv.) which was dissolved in methanol (1.3 mL) and then cyanogen bromide (3M in DCM, 0.14 mL, 0.414 mmol, 6 equiv.) was added. The vial was stirred at room temperature for 24 hours and solvent removed in vacuo to give 27d as the 2× HBr salt (57 mg, 0.0694 mmol, quantitative yield) as an off-white solid. MS (Method D, ESI+): m/z [M+H]⁺=664.3 (theoretical); 664.7 (observed). HPLC retention time: 0.95 min.

### Synthesis of 27e

An oven dried 4 mL vial with stir bar was charged with 27d (57 mg, 0.0694 mmol, 1 equiv.), 2-ethyl-5-methyl-pyrazole-3-carboxylic acid (43 mg, 0.278 mmol, 4 equiv.) and HATU (106 mg, 0.278 mmol, 4 equiv.) which were dissolved in DMF (1 mL) and then DIPEA (0.097 mL, 0.555 mmol, 8 equiv.) was added. The reaction was stirred at room temperature for 24 hours, quenched with 20% TFA in MeCN (200 uL) and product purified by prepHPLC (Method I, 5-95% MeCN in water with 0.05% TFA), solvent removed via lyophilization to give 27e as the 2×TFA salt (35 mg, 0.0302 mmol, 43% yield) as a tan solid. A sample of analytical purity was prepared via a second prepHPLC purification (Method G, 5-60% MeCN in water with 0.05% TFA). MS (Method D, ESI+): m/z [M+H]⁺=936.4 (theoretical); 936.3 (observed). HPLC retention time: 2.37 min.

### Synthesis of 27f

A 20 mL vial was charged with 27e (31 mg, 0.0266 mmol. 1 equiv.) which was dissolved in 20% TFA in DCM (2 mL) and stirred at room temperature for 15 minutes. Solvent was removed in vacuo and crude product purified by prepHPLC (Method H, 5-95% MeCN in water with 0.05% TFA) to give 27f as the 3×TFA salt (7.2 mg, 0.0061 mmol, 23% yield) as a white solid. MS (Method D, ESI+): m/z [M+H]⁺=836.4 (theoretical); 836.3 (observed). HPLC retention time: 2.02 min.

### Synthesis of 27

An oven-dried 4 mL vial was charged with 27f (10 mM in DMSO, 0.50 mL, 0.0050 mmol, 1 equiv.) and then MP-OSu (2.0 mg, 0.0075 mmol, 1.5 equiv.) and DIPEA (20 uL, 0.12 mmol, 23 equiv.) was added. The reaction was stirred at room temperature for 90 minutes, quenched with 20% TFA in MeCN (100 uL) and crude product was purified by prepHPLC (Method G, 5-95% MeCN in water with 0.05% TFA) to 27 as the 3×TFA salt (3.6 mg, 0.0029 mmol, 58% yield) as a white solid. MS (Method D, ESI+): m/z [M+H]⁺=987.4 (theoretical); 987.2 (observed). HPLC retention time: 2.23 min.

### Synthesis of S-(1-(3-((3-((5-carbamoyl-2-(1-ethyl-3-methyl-1H-pyrazole-5-carboxamido)-1-((E)-4-(2-(1-ethyl-3-methyl-1H-pyrazole-5-carboxamido)-5-sulfamoyl-1H-benzo[d]imidazol-1-yl)but-2-en-1-yl)-1H-benzo[d]imidazol-7-yl)oxy)propyl)(methyl)amino)-3-oxopropyl)-2,5-dioxopyrrolidin-3-yl)-L-cysteine (Compound 28)

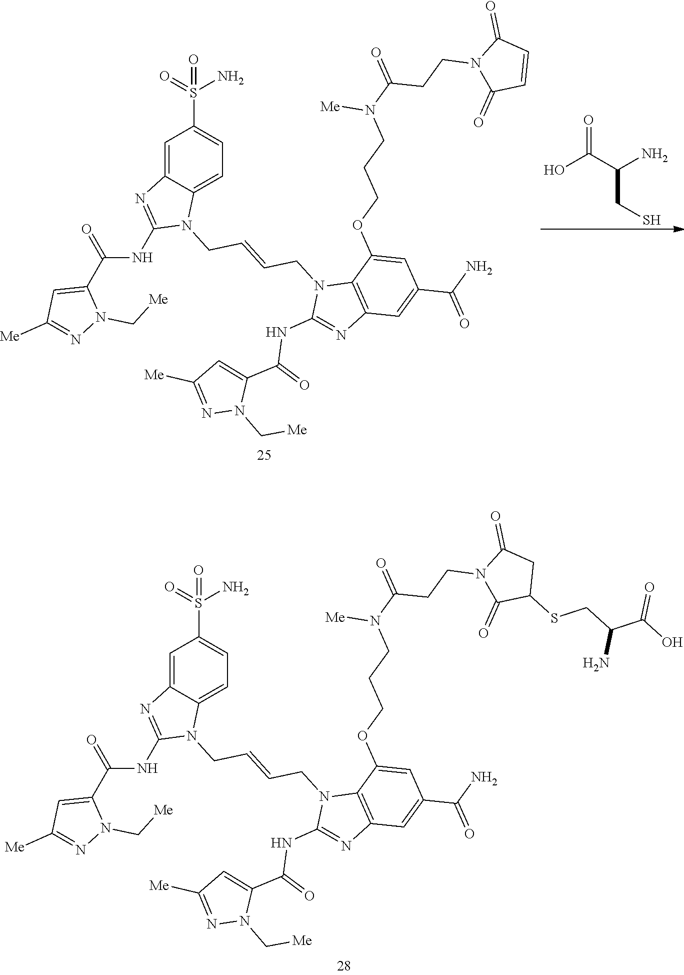 [SMILES: CCn1nc(C)cc1C(=O)Nc1nc2cc(S(N)(=O)=O)ccc2n1C/C=C/Cn1c(NC(=O)c2cc(C)nn2CC)nc2cc(C(N)=O)cc(OCCCN(C)C(=O)CCN3C(=O)C=CC3=O)c21 ; N[C@@H](CS)C(=O)O ; CCn1nc(C)cc1C(=O)Nc1nc2cc(S(N)(=O)=O)ccc2n1C/C=C/Cn1c(NC(=O)c2cc(C)nn2CC)nc2cc(C(N)=O)cc(OCCCN(C)C(=O)CCN3C(=O)CC(SC[C@H](N)C(=O)O)C3=O)c21]

A 1.7 mL eppendorf tube was charged with 25 (10 mM in DMSO, 100 uL, 0.00100 mmol, 1 equiv.) and L-cysteine (15 mM in 4:1 DMSO:water, 150 uL, 0.00300 mmol, 3 equiv.) was added. The reaction was heated to 37° C. for 90 minutes and the crude product was then purified by prepHPLC (Method G, 5-95% MeCN in water with 0.05% TFA) to give 28 as the 2×TFA salt (1.1 mg, 0.000861 mmol, 86% yield) as a white solid. MS (Method D, ESI+): m/z [M+H]⁺=1072.4 (theoretical); 1072.2 (observed). HPLC retention time: 1.98 min.

### Synthesis of S-(1-(3-(3-(2-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)ethyl)azetidin-1-yl)-3-oxopropyl)-2,5-dioxopyrrolidin-3-yl)-L-cysteine (Compound 29)

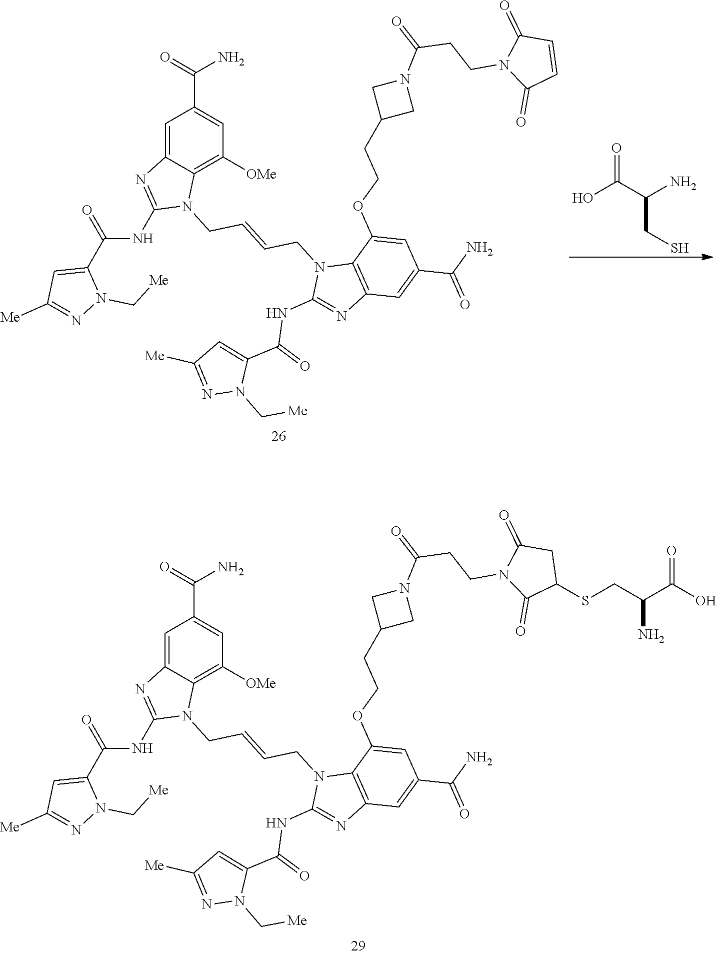 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCC3CN(C(=O)CCN4C(=O)C=CC4=O)C3)c21 ; N[C@@H](CS)C(=O)O ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCC3CN(C(=O)CCN4C(=O)CC(SC[C@H](N)C(=O)O)C4=O)C3)c21]

A 1.7 mL eppendorf tube was charged with 26 (10 mM in DMSO, 100 uL, 0.00100 mmol, 1 equiv.) and L-cysteine (15 mM in 4:1 DMSO:water, 150 uL, 0.00300 mmol, 3 equiv.) was added. The reaction was heated to 37° C. for 2 hours and the crude product was then purified by prepHPLC (Method G, 5-95% MeCN in water with 0.05% TFA) to give 29 as the 2× TFA salt (0.91 mg, 0.000697 mmol, 70% yield) as a white solid. MS (Method D, ESI+): m/z [M+H]⁺=1078.4 (theoretical); 1078.3 (observed). HPLC retention time: 2.03 min.

### S-(1-(3-(3-(3-((5-carbamoyl-1-((E)-4-(s-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propoxy)azetidin-1-yl)-3-oxopropyl)-2,5-dioxopyrrolidin-3-yl)-L-cysteine (Compound 30)

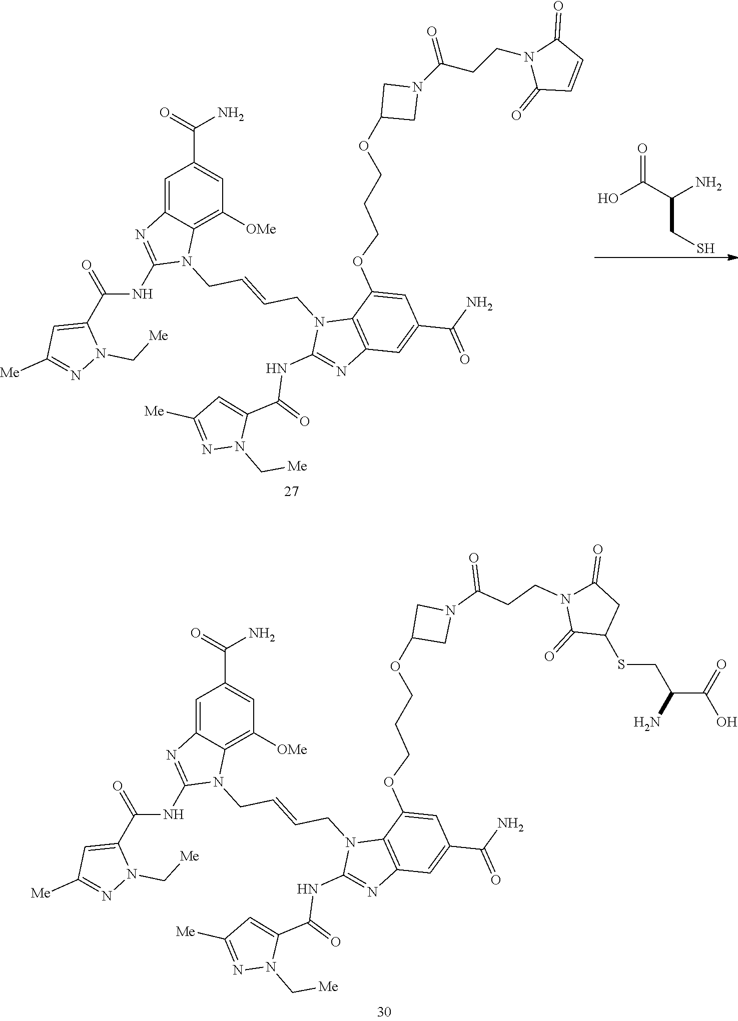 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCOC3CN(C(=O)CCN4C(=O)C=CC4=O)C3)c21 ; N[C@@H](CS)C(=O)O ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCOC3CN(C(=O)CCN4C(=O)CC(SC[C@H](N)C(=O)O)C4=O)C3)c21]

A 1.7 mL eppendorf tube was charged with 27 (10 mM in DMSO, 100 uL, 0.00100 mmol, 1 equiv.) and L-cysteine (100 mM in DMSO, 30 uL, 0.00300 mmol, 3 equiv.) and the solution incubated at 37° C. for 30 minutes. The crude product was purified by prepHPLC (Method G, 5-95% MeCN in water with 0.05%) to give 30 as the 2×TFA salt (1.2 mg, 0.000913 mmol, 61% yield) as a white solid. MS (Method D, ESI+): m/z [M+H]⁺=1108.4 (theoretical); 1108.5 (observed). HPLC retention time: 2.08 min.

Library Synthesis of Amide Analogs. Scheme and General Methods. Compounds 31-60.

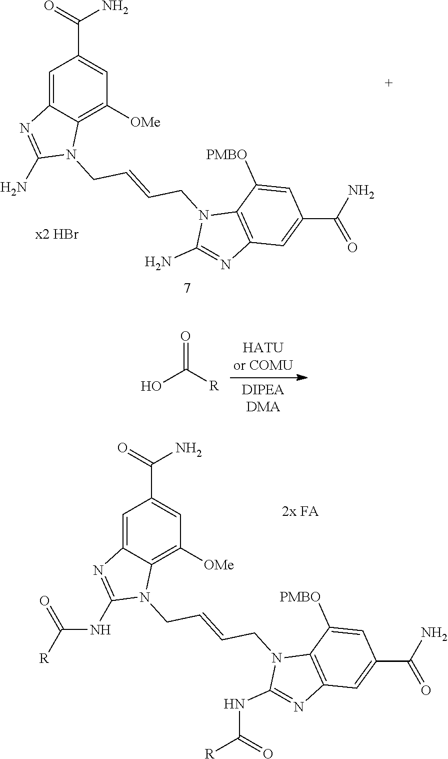

HATU Couplings (General Method 4A) To a solution of carboxylic acid (4 equiv.) in DMA (400 μL) was added HATU (6.2 mg, 0.016 mmol, 4 equiv.) and DIPEA (4.3 μL, 0.025 mmol, 6 equiv.). The mixture was stirred at room temperature for 30 minutes and then compound 7 (3 mg, 0.0041 mmol, 1 equiv.) was added to the mixture, and was heated to 70° C. for 18 hr. At which point, acetic acid (4.3 μL) was added, and resulting products were purified by prepHPLC (20-50-95% MeCN in water with 0.1% FA). All molecules were characterized using LC-MS Method D with ESI+ ionization.

COMU Couplings (General Method 4B) To a solution of carboxylic acid (4 equiv.) in DMA (400 μL) was added COMU (7 mg, 0.016 mmol, 4 equiv.) and DIPEA (4.3 μL, 0.025 mmol, 6 equiv.). The mixture was stirred at room temperature for 30 min and then compound 7 (3 mg, 0.0041 mmol, 1 equiv.) was added to the mixture, and the solution was heated to 40° C. for 18 hr. At which point, acetic acid was added (4.3 μL), and the resulting products were purified by prepHPLC (20-50-95% MeCN in water with 0.1% FA).

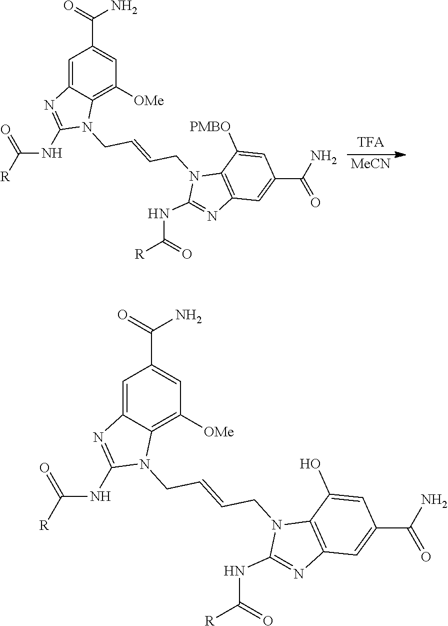

PMB deprotection (General Method 5) The resulting amide from the previous step was dissolved in 50% TFA in MeCN (0.01 M) and stirred at 30° C. for 30 min. Upon completion, the mixture was concentrated, and the product purified by prep-HPLC (20-50-95% water/acetonitrile 0.1% TFA).

Examples below were prepared using the general methods specified above.

| | | | Yield | PMB | Phenol |
|---|---|---|---|---|---|
| | | | (over 2 | LC-MS | LC-MS |
| Cmpd. | Structure | Method | steps) | data | data |
| 31 | 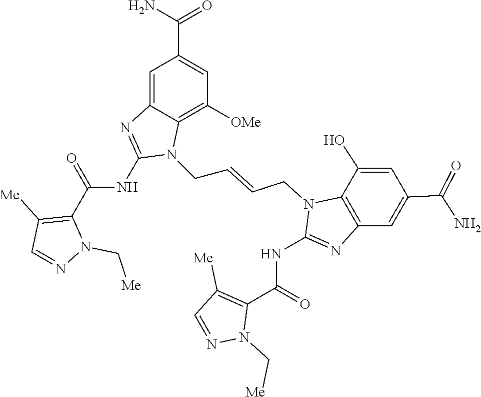 [SMILES: CCn1ncc(C)c1C(=O)Nc1nc2cc(C(N)=O)cc(O)c2n1C/C=C/Cn1c(NC(=O)c2c(C)cnn2CC)nc2cc(C(N)=O)cc(OC)c21] | HATU - Method 4a | 45% 1.74 mg 0.00183 mmol | RT: 1.49 Theoretical: 843.4 Observed: 843.5 | RT: 1.25 Theoretical: 723.3 Observed: 723.5 |
| 32 | 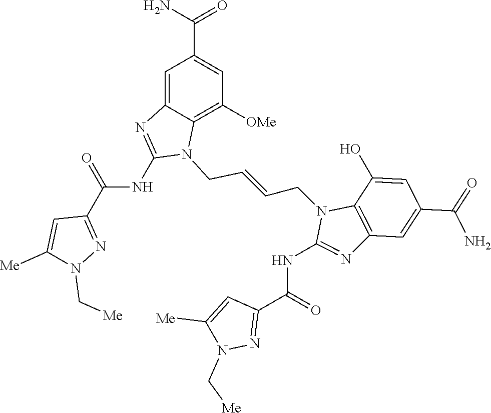 [SMILES: CCn1nc(C(=O)Nc2nc3cc(C(N)=O)cc(O)c3n2C/C=C/Cn2c(NC(=O)c3cc(C)n(CC)n3)nc3cc(C(N)=O)cc(OC)c32)cc1C] | HATU - Method 4a | 58% 2.25 mg 0.00237 mmol | RT: 1.67 Theoretical: 843.4 Observed: 843.5 | RT: 1.42 Theoretical: 723.3 Observed: 723.5 |
| 33 | 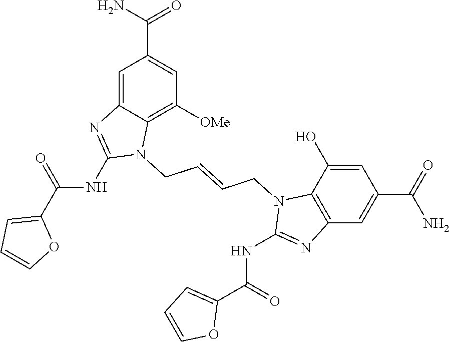 [SMILES: COc1cc(C(N)=O)cc2nc(NC(=O)c3ccco3)n(C/C=C/Cn3c(NC(=O)c4ccco4)nc4cc(C(N)=O)cc(O)c43)c12] | HATU - Method 4a | 56% 2.00 mg 0.00231 mmol | RT: 1.78 Theoretical: 759.2 Observed: 759.4 | RT: 1.60 Theoretical: 639.2 Observed: 639.3 |
| 34 | 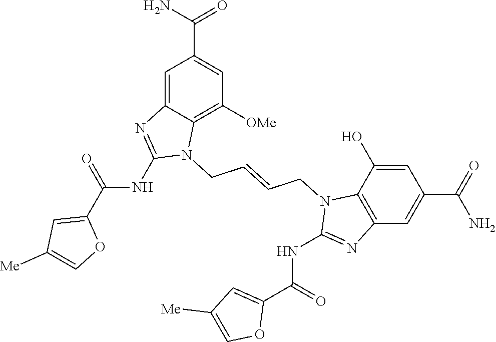 [SMILES: COc1cc(C(N)=O)cc2nc(NC(=O)c3cc(C)co3)n(C/C=C/Cn3c(NC(=O)c4cc(C)co4)nc4cc(C(N)=O)cc(O)c43)c12] | HATU - Method 4a | 61% 2.24 mg 0.00251 mmol | RT: 1.92 Theoretical: 787.3 Observed: 787.4 | RT: 1.74 Theoretical: 667.2 Observed: 667.4 |
| 35 | 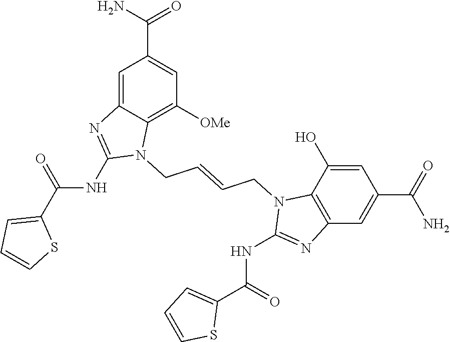 [SMILES: COc1cc(C(N)=O)cc2nc(NC(=O)c3cccs3)n(C/C=C/Cn3c(NC(=O)c4cccs4)nc4cc(C(N)=O)cc(O)c43)c12] | HATU - Method 4a | 59% 2.19 mg 0.00244 mmol | RT: 2.00 Theoretical: 791.2 Observed: 791.3 | RT: 1.85 Theoretical: 671.1 Observed: 671.3 |
| 36 | 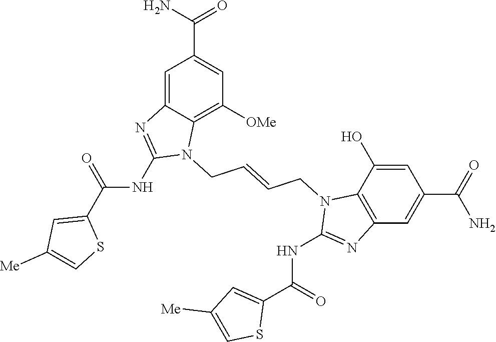 [SMILES: COc1cc(C(N)=O)cc2nc(NC(=O)c3cc(C)cs3)n(C/C=C/Cn3c(NC(=O)c4cc(C)cs4)nc4cc(C(N)=O)cc(O)c43)c12] | HATU - Method 4a | 40% 1.52 mg 0.00164 mmol | RT: 2.14 Theoretical: 819.2 Observed: 819.4 | RT: 2.01 Theoretical: 699.2 Observed: 699.3 |
| 37 | 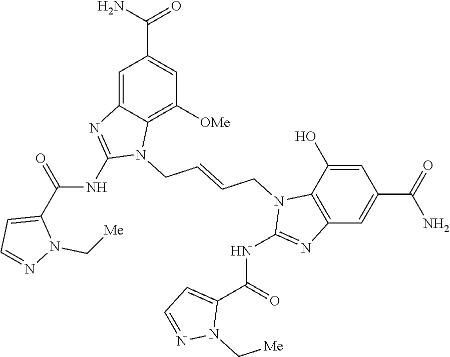 [SMILES: CCn1nccc1C(=O)Nc1nc2cc(C(N)=O)cc(O)c2n1C/C=C/Cn1c(NC(=O)c2ccnn2CC)nc2cc(C(N)=O)cc(OC)c21] | COMU - Method 4b | 35% 1.32 mg 0.00143 mmol | RT: 2.02 Theoretical: 815.3 Observed: 815.5 | RT: 1.63 Theoretical: 695.3 Observed: 695.4 |
| 38 | 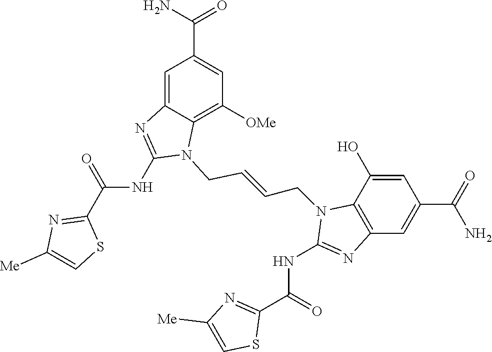 [SMILES: COc1cc(C(N)=O)cc2nc(NC(=O)c3nc(C)cs3)n(C/C=C/Cn3c(NC(=O)c4nc(C)cs4)nc4cc(C(N)=O)cc(O)c43)c12] | COMU - Method 4b | 39% 1.47 mg 0.00158 mmol | RT: 2.02 Theoretical: 821.2 Observed: 821.4 | RT: 1.63 Theoretical: 701.2 Observed: 701.3 |
| 39 | 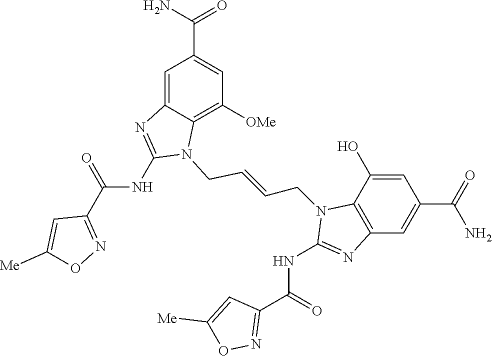 [SMILES: COc1cc(C(N)=O)cc2nc(NC(=O)c3cc(C)on3)n(C/C=C/Cn3c(NC(=O)c4cc(C)on4)nc4cc(C(N)=O)cc(O)c43)c12] | COMU - Method 4b | 57% 2.08 mg 0.00232 mmol | RT: 1.86 Theoretical: 789.3 Observed: 789.5 | RT: 1.65 Theoretical: 669.2 Observed: 669.4 |
| 40 | 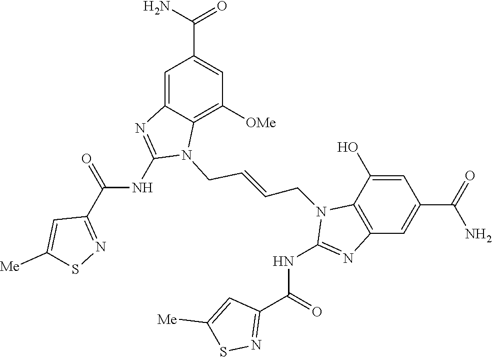 [SMILES: COc1cc(C(N)=O)cc2nc(NC(=O)c3cc(C)sn3)n(C/C=C/Cn3c(NC(=O)c4cc(C)sn4)nc4cc(C(N)=O)cc(O)c43)c12] | COMU - Method 4b | 36% 1.37 mg 0.00148 mmol | RT: 2.00 Theoretical: 821.2 Observed: 821.4 | RT: 1.61 Theoretical: 701.2 Observed: 701.3 |
| 41 | 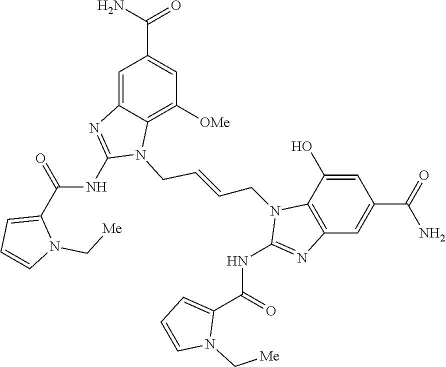 [SMILES: CCn1cccc1C(=O)Nc1nc2cc(C(N)=O)cc(O)c2n1C/C=C/Cn1c(NC(=O)c2cccn2CC)nc2cc(C(N)=O)cc(OC)c21] | HATU - Method 4a | 66% 2.50 mg 0.00272 mmol | RT: 2.24 Theoretical: 813.3 Observed: 813.5 | RT: 1.84 Theoretical: 693.3 Observed: 693.5 |
| 42 | 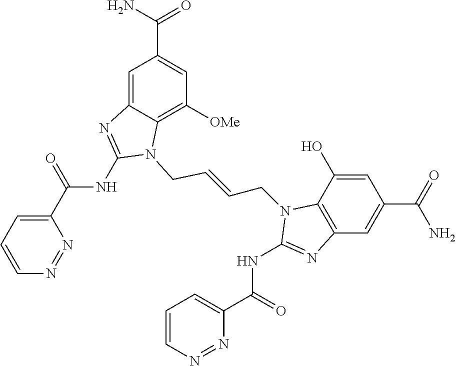 [SMILES: COc1cc(C(N)=O)cc2nc(NC(=O)c3cccnn3)n(C/C=C/Cn3c(NC(=O)c4cccnn4)nc4cc(C(N)=O)cc(O)c43)c12] | COMU - Method 4b | 45% 1.64 mg 0.00184 mmol | RT: 1.59 Theoretical: 783.3 Observed: 783.4 | RT: 1.31 Theoretical: 663.2 Observed: 663.4 |
| 43 | 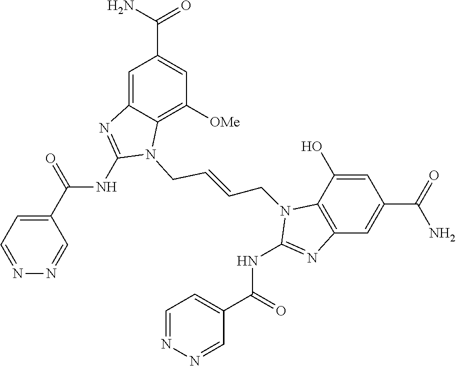 [SMILES: COc1cc(C(N)=O)cc2nc(NC(=O)c3ccnnc3)n(C/C=C/Cn3c(NC(=O)c4ccnnc4)nc4cc(C(N)=O)cc(O)c43)c12] | COMU - Method 4b | 29% 1.07 mg 0.00120 mmol | RT: 1.63 Theoretical: 783.3 Observed: 783.4 | RT: 1.36 Theoretical: 663.2 Observed: 663.4 |
| 44 | 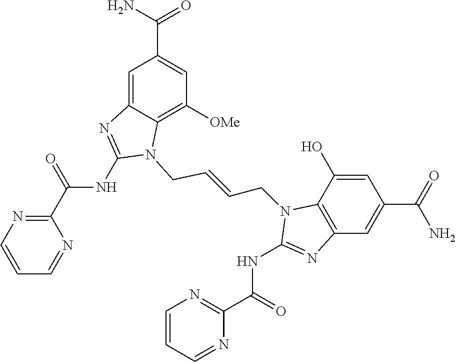 [SMILES: COc1cc(C(N)=O)cc2nc(NC(=O)c3ncccn3)n(C/C=C/Cn3c(NC(=O)c4ncccn4)nc4cc(C(N)=O)cc(O)c43)c12] | COMU - Method 4b | 55% 2.00 mg 0.00225 mmol | RT: 1.47 Theoretical: 783.3 Observed: 783.4 | RT: 1.19 Theoretical: 663.2 Observed: 663.4 |
| 45 | 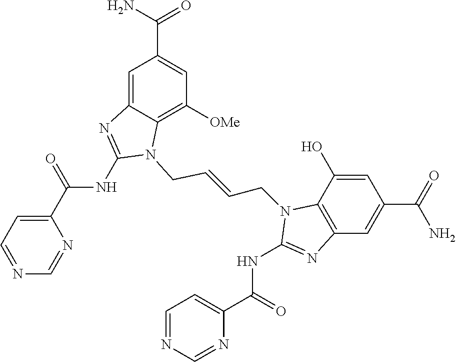 [SMILES: COc1cc(C(N)=O)cc2nc(NC(=O)c3ccncn3)n(C/C=C/Cn3c(NC(=O)c4ccncn4)nc4cc(C(N)=O)cc(O)c43)c12] | COMU - Method 4b | 37% 1.36 mg 0.00153 mg | RT: 1.61 Theoretical: 783.3 Observed: 783.4 | RT: 1.34 Theoretical: 663.2 Observed: 663.4 |
| 46 | 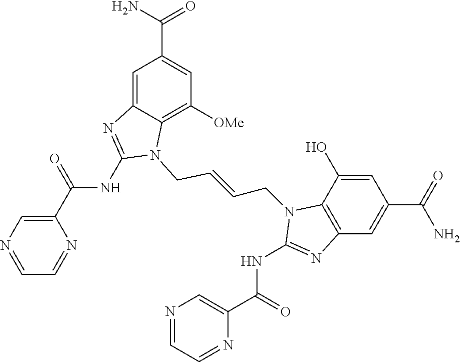 [SMILES: COc1cc(C(N)=O)cc2nc(NC(=O)c3cnccn3)n(C/C=C/Cn3c(NC(=O)c4cnccn4)nc4cc(C(N)=O)cc(O)c43)c12] | COMU - Method 4b | 63% 2.30 mg 0.00258 mmol | RT: 1.62 Theoretical: 783.3 Observed: 783.4 | RT: 1.34 Theoretical: 663.2 Observed: 663.4 |
| 47 | 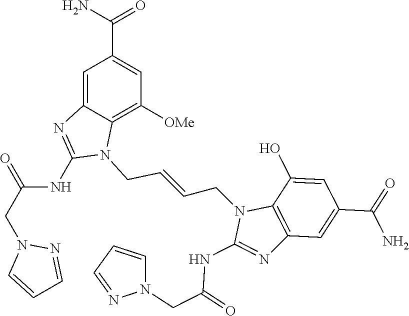 [SMILES: COc1cc(C(N)=O)cc2nc(NC(=O)Cn3cccn3)n(C/C=C/Cn3c(NC(=O)Cn4cccn4)nc4cc(C(N)=O)cc(O)c43)c12] | COMU - Method 4b | 46% 1.67 mg 0.00187 mmol | RT: 1.74 Theoretical: 787.3 Observed: 787.5 | RT: 1.46 Theoretical: 667.2 Observed: 667.4 |
| 48 | 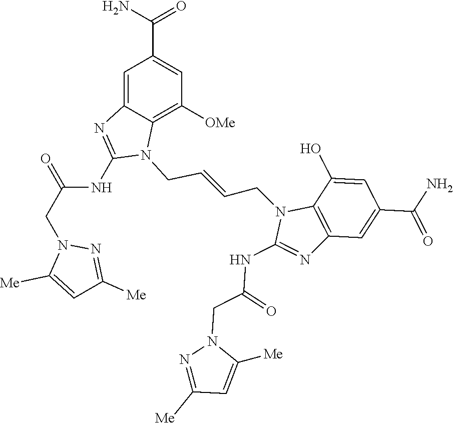 [SMILES: COc1cc(C(N)=O)cc2nc(NC(=O)Cn3nc(C)cc3C)n(C/C=C/Cn3c(NC(=O)Cn4nc(C)cc4C)nc4cc(C(N)=O)cc(O)c43)c12] | COMU - Method 4b | 60% 2.35 mg 0.00247 mmol | RT: 1.79 Theoretical: 843.4 Observed: 843.5 | RT: 1.52 Theoretical: 723.3 Observed: 723.5 |
| 49 | 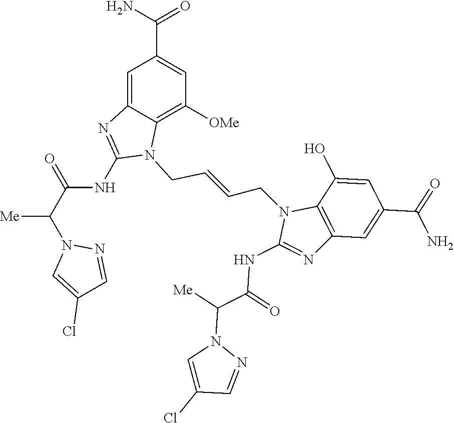 [SMILES: COc1cc(C(N)=O)cc2nc(NC(=O)C(C)n3cc(Cl)cn3)n(C/C=C/Cn3c(NC(=O)C(C)n4cc(Cl)cn4)nc4cc(C(N)=O)cc(O)c43)c12] | COMU - Method 4b | 60% 2.42 mg 0.00244 mmol | RT: 2.18 Theoretical: 883.3 Observed: 883.5 | RT: 1.90 Theoretical: 763.2 Observed: 763.4 |
| 50 | 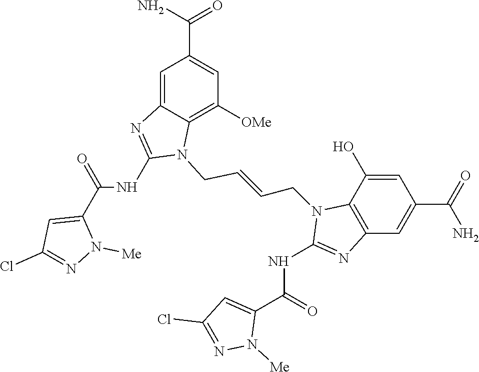 [SMILES: COc1cc(C(N)=O)cc2nc(NC(=O)c3cc(Cl)nn3C)n(C/C=C/Cn3c(NC(=O)c4cc(Cl)nn4C)nc4cc(C(N)=O)cc(O)c43)c12] | COMU - Method 4b | 55% 2.17 mg 0.00225 mmol | RT: 2.17 Theoretical: 855.2 Observed: 855.4 | RT: 1.59 Theoretical: 735.2 Observed: 735.3 |
| 51 | 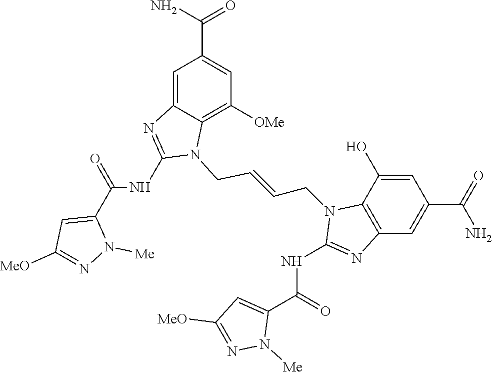 [SMILES: COc1cc(C(=O)Nc2nc3cc(C(N)=O)cc(O)c3n2C/C=C/Cn2c(NC(=O)c3cc(OC)nn3C)nc3cc(C(N)=O)cc(OC)c32)n(C)n1] | COMU - Method 4b | 71% 2.76 mg 0.00289 mmol | RT: 1.89 Theoretical: 847.3 Observed: 847.5 | RT: 1.31 Theoretical: 727.3 Observed: 727.6 |
| 52 | 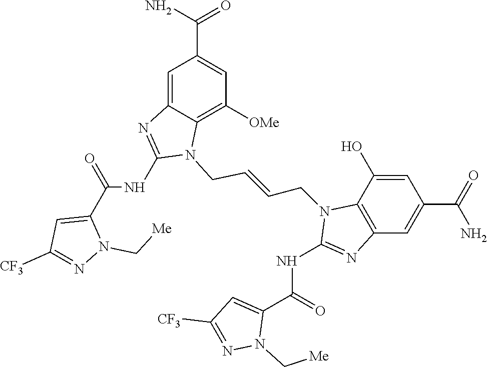 [SMILES: CCn1nc(C(F)(F)F)cc1C(=O)Nc1nc2cc(C(N)=O)cc(O)c2n1C/C=C/Cn1c(NC(=O)c2cc(C(F)(F)F)nn2CC)nc2cc(C(N)=O)cc(OC)c21] | COMU - Method 4b | 51% 2.23 mg 0.00211 mmol | RT: 2.50 Theoretical: 951.3 Observed: 951.6 | RT: 1.99 Theoretical: 831.3 Observed: 831.6 |
| 53 | 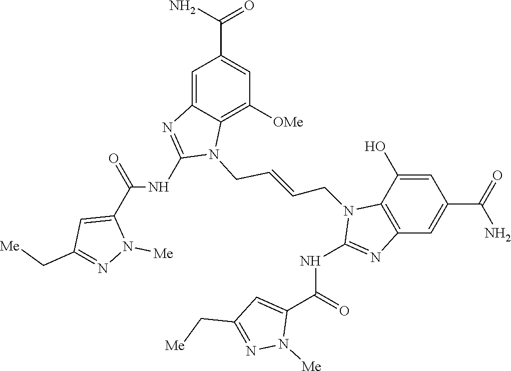 [SMILES: CCc1cc(C(=O)Nc2nc3cc(C(N)=O)cc(O)c3n2C/C=C/Cn2c(NC(=O)c3cc(CC)nn3C)nc3cc(C(N)=O)cc(OC)c32)n(C)n1] | COMU - Method 4b | 51% 2.00 mg 0.00210 mmol | RT: 2.01 Theoretical: 843.4 Observed: 843.5 | RT: 1.44 Theoretical: 723.3 Observed: 723.6 |
| 54 | 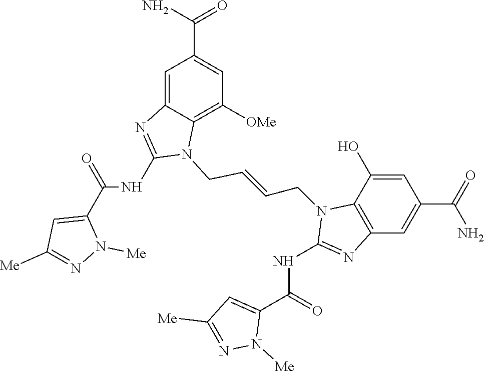 [SMILES: COc1cc(C(N)=O)cc2nc(NC(=O)c3cc(C)nn3C)n(C/C=C/Cn3c(NC(=O)c4cc(C)nn4C)nc4cc(C(N)=O)cc(O)c43)c12] | COMU - Method 4b | 52% 1.97 mg 0.00214 mmol | RT: 1.86 Theoretical: 815.3 Observed: 615.5 | RT: 1.28 Theoretical: 695.3 Observed: 695.5 |
| 55 | 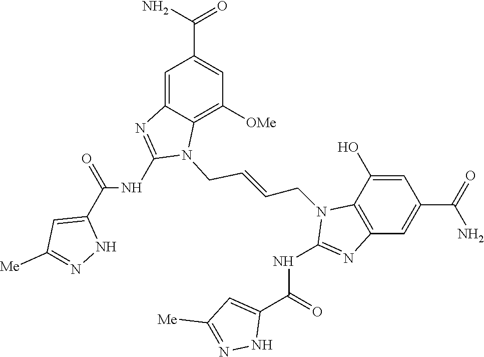 [SMILES: COc1cc(C(N)=O)cc2nc(NC(=O)c3cc(C)n[nH]3)n(C/C=C/Cn3c(NC(=O)c4cc(C)n[nH]4)nc4cc(C(N)=O)cc(O)c43)c12] | HATU - Method 4a | 56% 2.04 mg 0.00228 mmol | RT: 1.88 Theoretical: 1027.4 Observed: 1028.0 | RT: 1.35 Theoretical: 667.2 Observed: 667.8 |
| 56 | 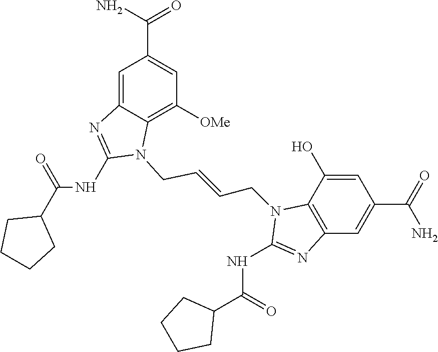 [SMILES: COc1cc(C(N)=O)cc2nc(NC(=O)C3CCCC3)n(C/C=C/Cn3c(NC(=O)C4CCCC4)nc4cc(C(N)=O)cc(O)c43)c12] | COMU - Method 4b | 63% 2.24 mg 0.00257 mmol | RT: 1.78 Theoretical: 763.4 Observed: 763.5 | RT: 1.11 Theoretical: 643.3 Observed: 643.9 |
| 57 | 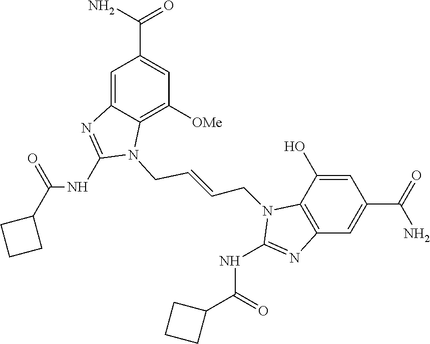 [SMILES: COc1cc(C(N)=O)cc2nc(NC(=O)C3CCC3)n(C/C=C/Cn3c(NC(=O)C4CCC4)nc4cc(C(N)=O)cc(O)c43)c12] | HATU - Method 4a | 60% 2.08 mg 0.00247 mmol | RT: 1.31 Theoretical: 735.3 Observed: 735.7 | RT: 0.99 Theoretical: 615.3 Observed: 615.6 |
| 58 | 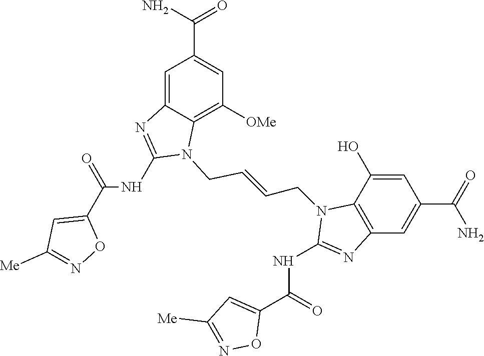 [SMILES: COc1cc(C(N)=O)cc2nc(NC(=O)c3cc(C)no3)n(C/C=C/Cn3c(NC(=O)c4cc(C)no4)nc4cc(C(N)=O)cc(O)c43)c12] | HATU - Method 4a | 55% 2.02 mg 0.00225 mmol | RT: 1.88 Theoretical: 789.3 Observed: 789.4 | RT: 1.60 Theoretical: 669.2 Observed: 669.3 |
| 59 | 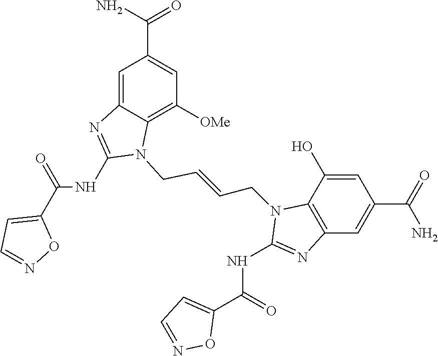 [SMILES: COc1cc(C(N)=O)cc2nc(NC(=O)c3ccno3)n(C/C=C/Cn3c(NC(=O)c4ccno4)nc4cc(C(N)=O)cc(O)c43)c12] | HATU - Method 4a | 56% 2.00 mg 0.00230 mmol | RT: 1.81 Theoretical: 761.2 Observed: 761.4 | RT: 1.56 Theoretical: 641.2 Observed: 641.3 |
| 60 | 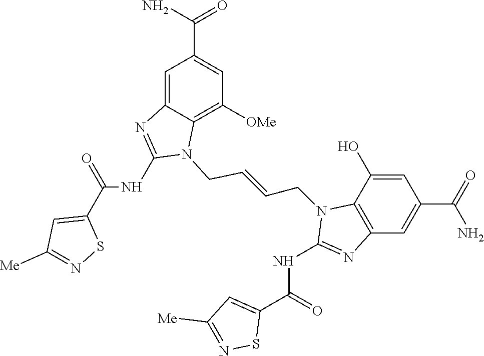 [SMILES: COc1cc(C(N)=O)cc2nc(NC(=O)c3cc(C)ns3)n(C/C=C/Cn3c(NC(=O)c4cc(C)ns4)nc4cc(C(N)=O)cc(O)c43)c12] | HATU - Method 4a | 48% 1.82 mg 0.00196 mmol | RT: 1.98 Theoretical: 821.2 Observed: 821.4 | RT: 1.68 Theoretical: 701.2 Observed: 701.4 |

### Synthesis of methyl 4-chloro-3-((4-methoxybenzyl)oxy)-5-nitrobenzoate (Compound 61)

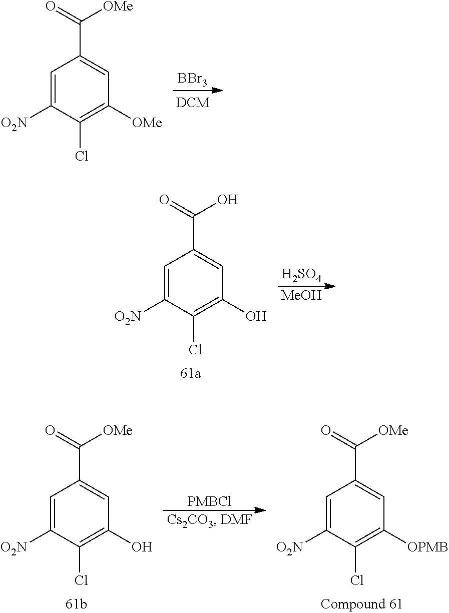

### Synthesis of 61a

To a solution of methyl 4-chloro-3-methoxy-5-nitrobenzoate (15 g, 61 mmol, 1 equiv.) in DC (60 mL) at 0° C. under nitrogen was added BBr₃ (1 M in DCM, 153 mL, 153 mmols, 2.5 equiv.) dropwise over 20 min. The reaction mixture was stirred at 0° C. for 30 min and then allowed to warm to 25° C. and stirred for a further 12 h. The reaction mixture was cooled to 0° C., quenched with methanol, and concentrated in vacuo to give 61a (12.3 g, 56.5 mmols, 93% yield) as dark brown oil. LC-MS (Method C, ESI+): m/z [M+H]⁺=218.0 (theoretical); 217.9 (observed). HPLC retention time: 0.21 min.

### Synthesis of 61b

To a solution of 61a (26.6 g, 122 mmol, 1 equiv.) in methanol (800 mL), was added concentrated H₂SO₄ (600 mg, 6.11 mmol, 0.05 equiv.), the mixture was stirred at 60° C. for 12 h. LCMS analysis (Method C) showed the reaction was completed. The mixture was cooled to room temperature and concentrated in vacuo. The crude residue was diluted with water (50 mL) and saturated NaHCO₃ (50 mL) was carefully added to achieve a pH >7. The resultant solid was collected by filtration, washed with water (25 mL) and dried under vacuum to give 61b (25 g, 88% yield) as a brown solid. LC-MS (Method C, ESI+): m/z [M+H]⁺=232.0 (theoretical); 231.9 (observed). HPLC retention time: 0.92 min.

### Synthesis of 61

To a solution of 61b (18 g, 78 mmol, 1 equiv.) in DMF (200 mL) was added Cs₂CO₃ (27.9 g, 86 mmol, 1.1 equiv.) and 1-(chloromethyl)-4-methoxybenzene (12.8 g, 82 mmol, 1.05 equiv.) and the mixture was stirred at 25° C. for 16 h. LCMS analysis (Method C) showed the reaction was completed. The reaction was poured into water, filtered and dried under high vacuum to give 61 (22.3 g, 82% yield) as a light-yellow solid. ¹H NMR (400 MHz, DMSO-d₆): δ=8.11 (d, J=1.4 Hz, 1H), 7.97 (d, J=1.4 Hz, 1H), 7.43 (d, J=8.5 Hz, 2H), 6.99 (d, J=8.5 Hz, 2H), 5.33 (s, 2H), 3.92 (s, 3H), 3.77 (s, 3H).

### Synthesis of methyl (E)-2-(1-ethyl-3-methyl-1H-pyrazole-5-carboxamido)-1-(4-(2-(1-ethyl-3-methyl-1H-pyrazole-5-carboxamido)-7-hydroxy-5-(methoxycarbonyl)-1H-benzo[d]imidazol-1-yl)but-2-en-1-yl)-7-methoxy-1H-benzo[d]imidazole-5-carboxylate (Compound 62)

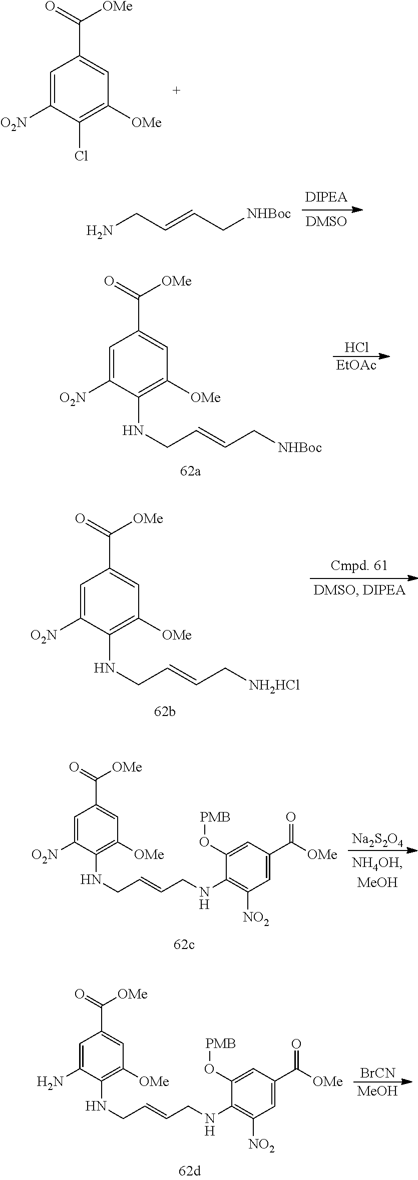

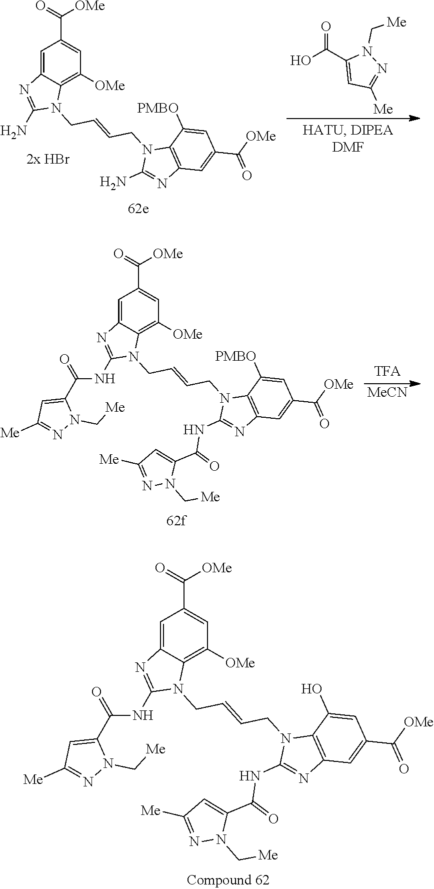

### Synthesis of 62a

To a solution of tert-butyl (E)-(4-aminobut-2-en-1-yl)carbamate (12.5 g, 67.2 mmol, 1.1 equiv.) in DMSO (150 mL) was added methyl 4-chloro-3-methoxy-5-nitrobenzoate (15 g, 61.1 mmol, 1 equiv.) and DIPEA (39.5 g, 305 mmol, 5 equiv.) the mixture was stirred at 100° C. for 12 h. The mixture was poured into water, extracted with EtOAc and concentrated in vacuo to give 62a (16.4 g, 41.4 mmols, 68% yield) as a dark red solid. LC-MS (Method C, ESI+): m/z [M −tBu]⁺=340.1 (theoretical); 340.1 (observed). HPLC retention time: 1.08 min.

### Synthesis of 62b

62a (21 g, 53.1 mmol, 1 equiv.) was added to a solution of HCl in ethyl acetate (4 M, 350 mL, 1400 mmols, 26 equiv.) and the mixture was stirred at 25° C. for 2 h. The mixture was concentrated in vacuo and crude solid washed with EtOAc to give 62b as the HCl salt (14.5 g, 43.7 mmols, 82% yield) as a dark red solid. ¹H NMR (400 MHz, DMSO-d₆): δ=8.19 (d, J=1.8 Hz, 1H), 8.12 (br s, 1H), 8.01 (br s, 3H), 7.46 (d, J=1.6 Hz, 1H), 5.87 (td, J=5.8, 15.5 Hz, 1H), 5.71-5.55 (m, 1H), 4.21 (br s, 2H), 3.90 (s, 3H), 3.84 (s, 3H), 3.42-3.35 (m, 2H).

### Synthesis of 62c

To a solution of 61 (4.5 g, 12.8 mmol) in DMSO (70 mL) was added 62b (4.67 g, 14.1 mmol, HCl salt) and DIPEA (8.3 g, 64 mmol, 5 equiv.) and the reaction was stirred at 80° C. for 10 h. The mixture was poured into ice water, extracted with EtOAc and concentrated in vacuo. The residue was recrystallized (ethyl acetate, 20V, reflux) to give 62c (6.4 g, 10.5 mmols, 82% yield) as a dark red solid. MS (Method C, ESI+): m/z [M+H]⁺=611.2 (theoretical); 611.2 (observed). HPLC retention time: 1.34 min. ¹H NMR (400 MHz, DMSO-d₆): δ=8.06 (dd, J=1.5, 9.5 Hz, 2H), 7.96 (br d, J=2.9 Hz, 2H), 7.44 (s, 1H), 7.36 (d, J=8.5 Hz, 2H), 7.30 (s, 1H), 6.94 (d, J=8.5 Hz, 2H), 5.53-5.29 (m, 2H), 5.00 (s, 2H), 4.03 (br t, J=5.4 Hz, 4H), 3.84 (s, 6H), 3.76 (d, J=3.5 Hz, 6H).

### Synthesis of 62d

To a solution of 62c (6.0 g, 9.83 mmol, 1 equiv.) in MeOH (300 mL) was added NH₄OH (60 mL, 28% NH₃ in H₂O) and Na₂S₂O₄ (20.5 g, 118 mmol, 12 equiv.). The mixture was stirred at 25° C. for 16 h and went from bright red to a light yellow/nearly colorless heterogenous mixture. The mixture was filtered, concentrated to remove MeOH and the remaining aqueous solution was extracted with EtOAc. The organic phases were combined, dried with Na₂SO₄ and concentrated in vacuo to give 62d (4.0 g, 7.25 mmols, 74% yield) as an off white solid. MS (Method B, ESI+): m/z [M+H]⁺=551.25 (theoretical); 551.2 (observed). HPLC retention time: 1.29 min.

### Synthesis of 62e

To a solution of 62d (4.0 g, 7.25 mmol, 1 equiv.) in MeOH (200 mL) was added BrCN (4.62 g, 43.6 mmol, 6 equiv.). The mixture was stirred at 25° C. for 2 h at which point LC-MS analysis (Method C) showed full conversion. The reaction mixture was concentrated in vacuo and the crude product washed with ethanol and petroleum ether to give 62e as the 2× HBr salt (2.6 g, 3.53 mmols 49% yield) as an off white solid. MS (Method C, ESI+): m/z [M+H]⁺=601.2 (theoretical); 601.3 (observed). HPLC retention time: 2.73 min. ¹H NMR (400 MHz, DMSO-d₆): δ=12.87 (br s, 1H), 8.72 (br d, J=17.0 Hz, 4H), 7.59 (s, 2H), 7.42 (s, 1H), 7.26-7.16 (m, 3H), 6.82 (d, J=8.6 Hz, 2H), 5.70 (br d, J=15.7 Hz, 1H), 5.57-5.48 (m, 1H), 5.00 (s, 2H), 4.83-4.73 (m, 4H), 3.88 (s, 6H), 3.71 (s, 3H), 3.65 (s, 3H).

### Synthesis of 62f

To a solution of 1-ethyl-3-methyl-1H-pyrazole-5-carboxylic acid (3.15 g, 20.5 mmol, 2.6 equiv.) in DMF (30 mL) was added HATU (8.38 g, 22.0 mmol, 2.8 equiv.) and the solution was stirred at 60° C. for 10 min. A second solution containing DIPEA (5.09 g, 39 mmol, 5 equiv.) and 62e (6.0 g, 7.87 mmol, 1 equiv. 2× HBr salt) in DMF, 30 mL) was prepared and added to the activated acid. The reaction was then stirred at 60° C. for 2 h. The solution was poured into water, filtered and triturated with acetonitrile to give 62f (2.54 g, 2.91 mmols, 37% yield) as an off-white solid. MS (Method C, ESI+): m/z [M+H]⁺=873.4 (theoretical); 873.4 (observed). HPLC retention time: 3.44 min. ¹H NMR (400 MHz, DMSO-d₆) δ=12.88 (br s, 2H), 7.74 (br s, 2H), 7.22 (br s, 1H), 7.16-6.97 (m, 3H), 6.66 (br d, J=7.9 Hz, 2H), 6.57-6.36 (m, 2H), 5.87-5.37 (m, 2H), 4.78 (br s, 6H), 4.51 (br dd, J=7.0, 17.3 Hz, 4H), 3.85 (s, 6H), 3.59 (s, 3H), 3.52 (br s, 3H), 2.10 (br d, J=11.1 Hz, 6H), 1.26 (td, J=6.8, 18.8 Hz, 6H).

### Synthesis of 62

An oven-dried 4 mL vial with stir bar was charged with 62f (9 mg, 0.010 mmols, 1 equiv.) which was dissolved in 1:1 MeCN:TFA (1 mL) and stirred for 1 hour at room temperature. Solvent was removed in vacuo and product dried on high-vac overnight to give 62 (7.5 mg, 0.0099 mmols, quant. yield) as a tan solid. MS (Method D, ESI+): m/z [M+H]⁺=753.3 (theoretical); 753.7 (observed). HPLC retention time: 1.99 min.

### Synthesis of (E)-1-(4-(5-carboxy-2-(1-ethyl-3-methyl-1H-pyrazole-5-carboxamido)-7-hydroxy-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxylic acid (Compound 63)

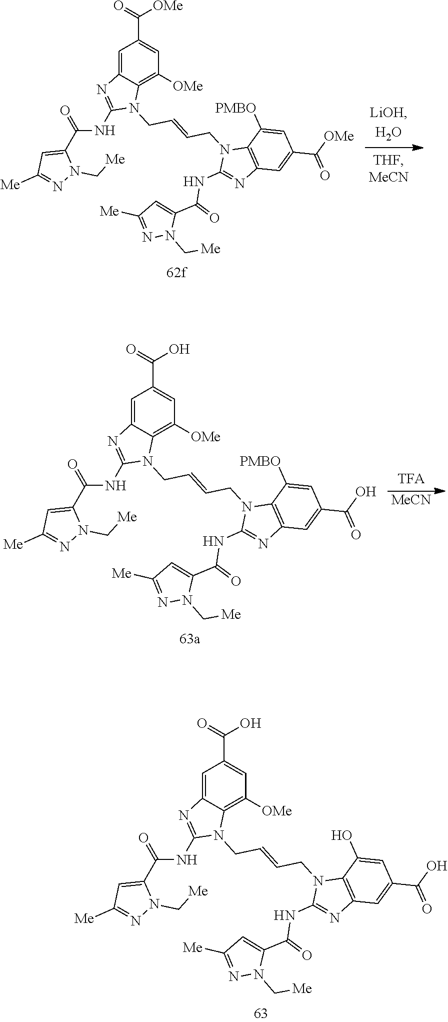

### Synthesis of 63a

Compound 62f (100 mg, 0.115 mmols, 1 equiv.) was dissolved in acetonitrile (1 mL), 1M LiOH (1 mL, 1 mmol, 9 equiv.) was added and the solution was heated to 80° C. for 1 hour. The vial was cooled, solvent removed in vacuo and product purified by prepHPLC (Method I, 5-95% MeCN in water with 0.1% TFA) to give 63a (78 mg, 0.092 mmols, 97% yield) as a white solid. MS (Method D, ESI+): m/z [M+H]⁺=845.3 (theoretical); 845.8 (observed). HPLC retention time: 1.95 min.

### Synthesis of 63

Compound 63 was prepared as previously described (see “Synthesis of 62”). MS (Method D, ESI+): m/z [M+H]⁺=725.3 (theoretical); 725.4 (observed). HPLC retention time: 1.83 min.

### Synthesis of (E)-2-(1-ethyl-3-methyl-1H-pyrazole-5-carboxamido)-1-(4-(2-(1-ethyl-3-methyl-1H-pyrazole-5-carboxamido)-7-hydroxy-5-(methoxycarbonyl)-1H-benzo[d]imidazol-1-yl)but-2-en-1-yl)-7-methoxy-1H-benzo[d]imidazole-5-carboxylic acid (Compound 64) and (E)-2-(1-ethyl-3-methyl-1H-pyrazole-5-carboxamido)-1-(4-(2-(1-ethyl-3-methyl-1H-pyrazole-5-carboxamido)-7-methoxy-5-(methoxycarbonyl)-1H-benzo[d]imidazol-1-yl)but-2-en-1-yl)-7-hydroxy-1H-benzo[d]imidazole-5-carboxylic acid Compound 65)

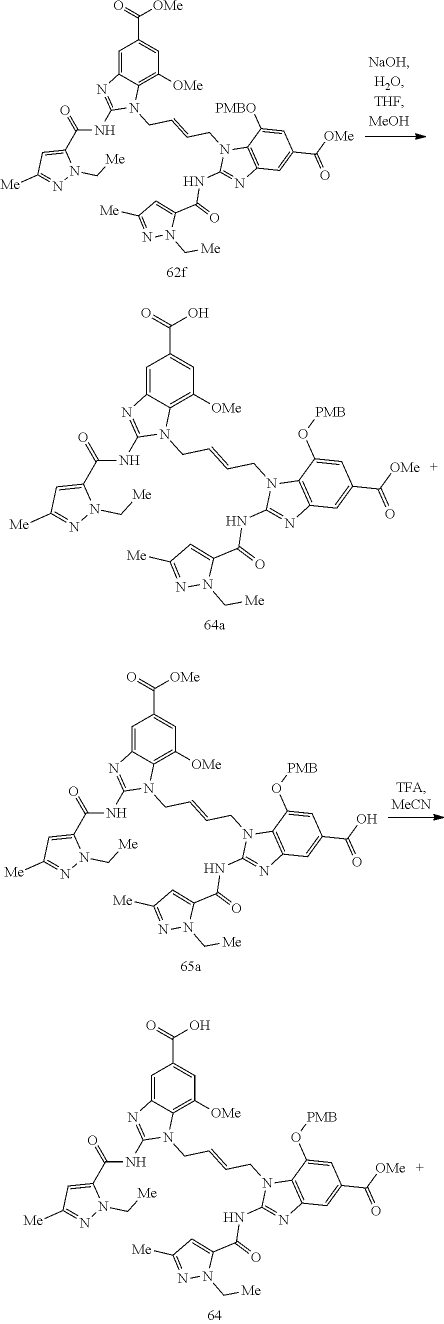

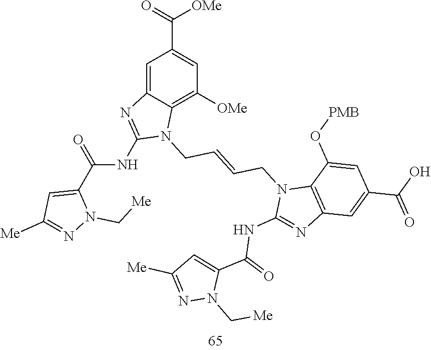

### Synthesis of 64a and 65a

An inseparable 1:1 mixture of compounds 64a and 65a was prepared as previously described (see “Synthesis of 65a”) substituting sodium hydroxide for lithium hydroxide and quenching the reaction at 50% conversion followed by purification via prepHPLC (Method H with 0.1% FA). MS (Method D, ESI+): m/z [M+H]+=859.4 (theoretical); 859.5 (observed). HPLC retention time: 2.46 min.

### Synthesis of 6 and 65

An inseparable 1:1 mixture of compounds 64a and 65a was prepared as previously described (see “Synthesis of 65a”). MS (Method D, ESI+): m/z [M+H]+=739.4 (theoretical); 739.4 (observed). HPLC retention time: 1.99 and 2.04 min.

### Synthesis of methyl (E)-7-(3-(3-(2,5-dioxo-2,5-dihydro-1H-pyrrol-1-yl)-N-methylpropanamido)propoxy)-2-(1-ethyl-3-methyl-1H-pyrazole-5-carboxamido)-1-(4-(2-(1-ethyl-3-methyl-1H-pyrazole-5-carboxamido)-7-methoxy-5-(methoxycarbonyl)-1H-benzo[d]imidazol-1-yl)but-2-en-1-yl)-1H-benzo[d]imidazole-5-carboxylate (Compound 66)

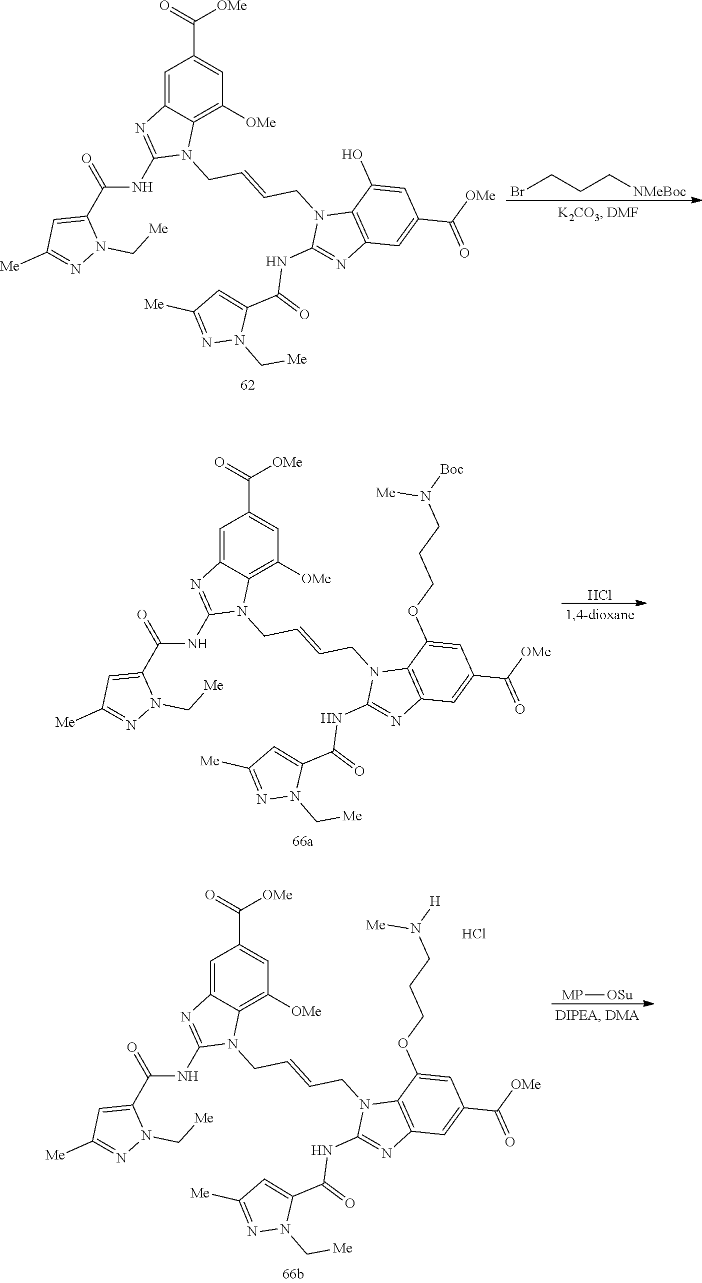

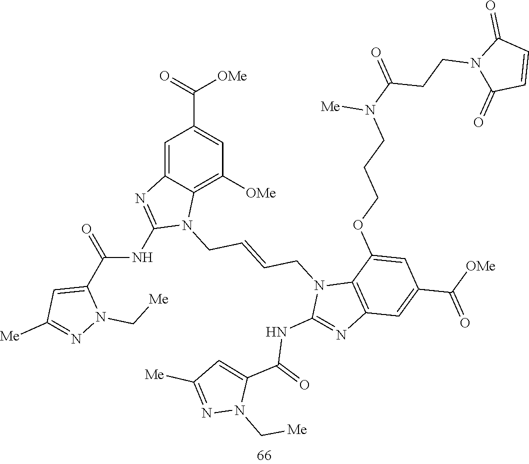 [SMILES: CCn1nc(C)cc1C(=O)Nc1nc2cc(C(=O)OC)cc(OC)c2n1C/C=C/Cn1c(NC(=O)c2cc(C)nn2CC)nc2cc(C(=O)OC)cc(OCCCN(C)C(=O)CCN3C(=O)C=CC3=O)c21]

### Synthesis of 66a

Compound 62 (397 mg, 0.527 mmol, 1 equiv.), tert-butyl (3-bromopropyl)(methyl)carbamate (146 mg, 0.580 mmol, 1.1 equiv.) and potassium carbonate (218 mg, 1.58 mmol, 3 equiv.) were dissolved in DMF (5.3 mL) in a 20 mL vial. The reaction was stirred at 55° C. for 18 hours and then the mixture was filtered washing with methanol and the filtrate concentrated in vacuo. To the crude solid was added cold water and the precipitate isolated via filtration to give 66a (232 mg, 0.251 mmol, 48% yield). MS (Method E, ESI+): m/z [M+H]⁺=924.4 (theoretical); 924.9 (observed). HPLC retention time: 2.42 min.

### Synthesis of 66b

Compound 66a (232 mg, 0.251 mmol, 1 equiv.) was dissolved in methanol (2.5 mL) and 4M HCl in dioxane was added (0.5 mL, 2.01 mmol, 8 equiv.). The reaction stirred at 30° C. for 90 minutes. The solvent was in vacuo and the crude product purified by prepHPLC (Method I with 0.05% TFA) to afford 66b (206 mg, 0.24 mmol, 96% yield). MS (Method E, ESI+): m/z [M+H]⁺=824.4 (theoretical); 824.9 (observed). HPLC retention time: 1.56 min.

### Synthesis of 66

Compound 66 (25 mg, 0.0291 mmol, 1 equiv.) and MP-OSu (11.6 mg, 0.0436 mmol, 1.5 equiv.) were dissolved in DMA (0.58 mL) and DIPEA (20 μL, 0.116 mmol) was added. The reaction was stirred at room temperature for 1 hour. The mixture was directly purified by prepHPLC (Method H, with 0.05% TFA) to afford 66 as a white solid (10.88 mg, 0.0112 mmol, 38% yield). MS (Method D, ESI+): m/z [M+H]⁺=975.4 (theoretical); 975.4 (observed). HPLC retention time: 2.24 min.

### Synthesis of (2S,3S,4S,5R,6S)-6-(3-(3-(3-(2,5-dioxo-2,5-dihydro-1H-pyrrol-1-yl)propanamido)propanamido)-4-((((3-((2-(1-ethyl-3-methyl-1H-pyrazole-5-carboxamido)-1-((E)-4-(2-(1-ethyl-3-methyl-1H-pyrazole-5-carboxamido)-7-methoxy-5-(methoxycarbonyl)-1H-benzo[d]imidazol-1-yl)but-2-en-1-yl)-5-(methoxycarbonyl)-1H-benzo[d]imidazol-7-yl)oxy)propyl)(methyl)carbamoyl)oxy)methyl)phenoxy)-3,4,5-trihydroxytetrahydro-2H-pyran-2-carboxylic acid (Compound 67)

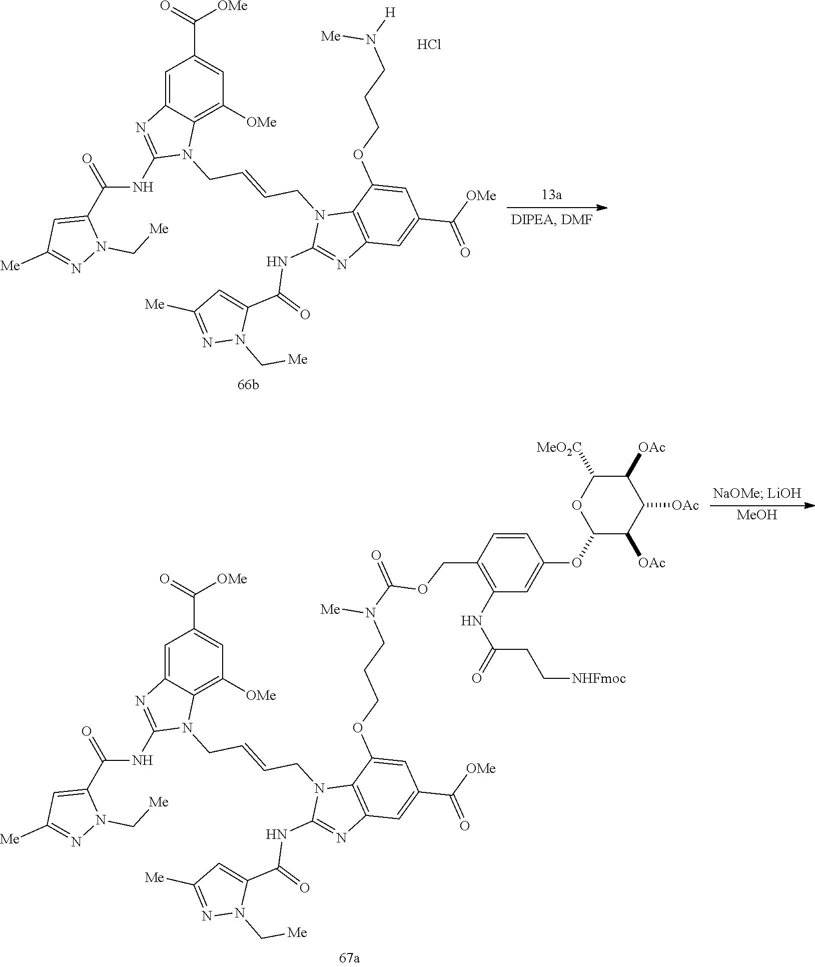

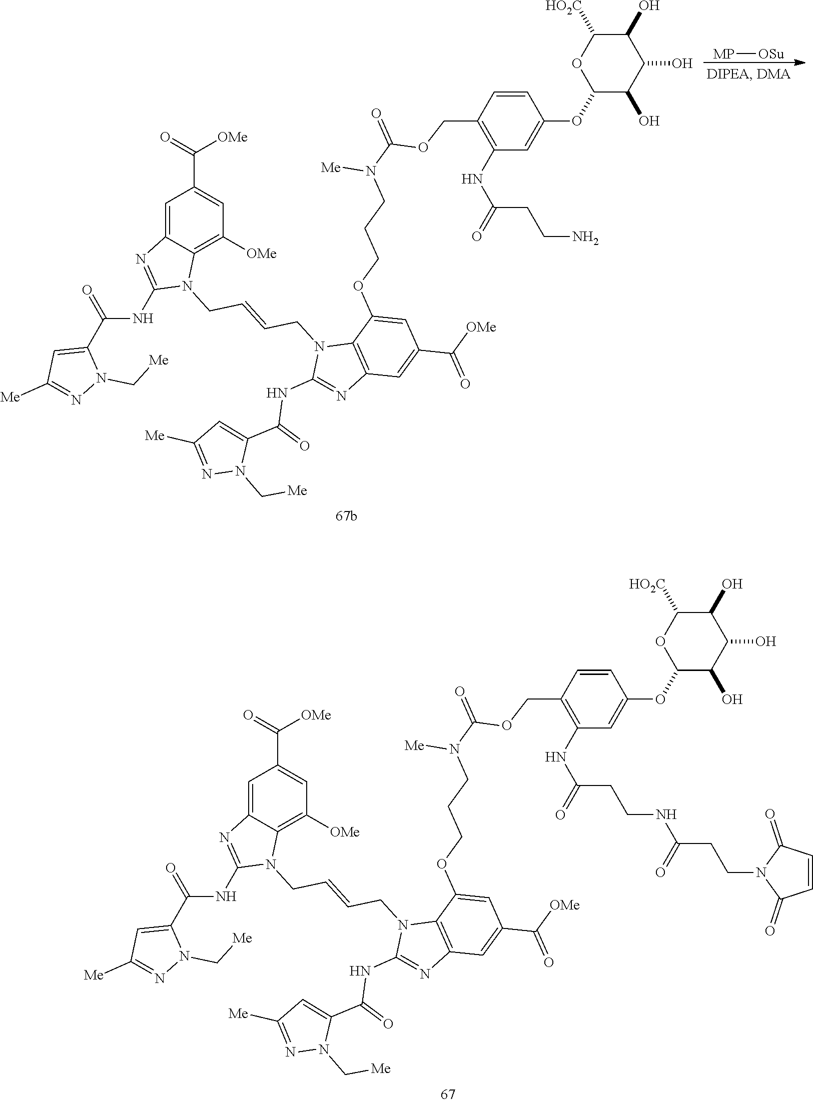

### Synthesis of 67a

Compound 13a (65 mg, 0.0868 mmol, 1.4 equiv.) and bis(pentafluorophenyl) carbonate (120 mg, 0.304 mmo, 5 equiv.) were dissolved in DMA (0.43 mL) and DIPEA (70 μL, 0.404 mmol, 6.7 equiv.) was added. The reaction was stirred for 30 minutes and then 66b (52 mg, 0.0607 mmol, 1 equiv.) was added. The reaction was stirred at room temperature for 18 hours. The solution was diluted with H₂O and extracted with EtOAc (3×), and the combined organics were washed with 1M HCl (3×), dried with MgSO₄, filtered and solvent removed in vacuo to give a crude solid. This material was dissolved in DMSO and purified by prepHPLC (Method H, with 0.05% TFA) to give 67a as a white solid (33.1 mg, 0.0207 mmol, 34% yield). LCMS (Method D, ESI+) m/z [M+H]⁺ 1598.6 (theoretical), 1598.6 (observed). LCMS retention time 2.65 min. MS (Method D, ESI+): m/z [M+H]⁺=1598.6 (theoretical); 1598.6 (observed). HPLC retention time: 2.65 min.

### Synthesis of 67b

Compound 67a (33.1 mg, 0.0207 mmol) was dissolved in dry methanol (0.21 mL), cooled in an ice bath, and 0.5M NaOMe in MeOH (41.5 μL, 0.0414 mmol, 2 equiv.) was added. The reaction was monitored by LCMS (Method D) and upon full acetate deprotection, 1M LiOH (62 μL, 0.0621 mmol, 3 equiv.) was added. The reaction stirred at room temperature for 1h monitoring by LCMS (Method E). Upon full conversion, acetic acid (62 μL) was added, solvent removed in vacuo and crude product purified via prepHPLC (Method H, with 0.05% TFA) to give 67b as a white solid (10.1 mg, 0.0075 mmol, 36% yield). LCMS (Method D, ESI+) m/z [M+H]⁺1236.5 (theoretical), 1236.5 (observed). LCMS retention time 2.31 min.

MS (Method D, ESI+): m/z [M+H]⁺=1236.5 (theoretical); 1236.5 (observed). HPLC retention time: 2.31 min.

### Synthesis of 67

Compound 67b (10.1 mg, 0.0075 mmol, 1 equiv.) and MP-OSu (3.0 mg, 0.0112 mmol, 1.5 equiv.) were dissolved in DMA (150 μL), DIPEA (4 μL, 0.0224 mmol) was added. The reaction was stirred for 30 min at room temperature at which point acetic acid (4 μL) was added, and the mixture was purified via prepHPLC (Method G, with 0.05% TFA) to obtain 67 as a white solid (3.3 mg, 0.0024 mmol, 32% yield). LCMS (Method E, ESI+) m/z [M+H]⁺ 1387.5 (theoretical), 1387.5 (observed). LCMS retention time 1.92 min.

MS (Method E, ESI+): m/z [M+H]⁺=1387.5 (theoretical); 1387.5 (observed). HPLC retention time: 1.92 min.

### Synthesis of (E)-1-(4-(5-carboxy-2-(1-ethyl-3-methyl-1H-pyrazole-5-carboxamido)-7-methoxy-1H-benzo[d]imidazol-1-yl)but-2-en-1-yl)-7-(3-(3-(2,5-dioxo-2,5-dihydro-1H-pyrrol-1-yl)-N-methylpropanamido)propoxy)-2-(1-ethyl-3-methyl-1H-pyrazole-5-carboxamido)-1H-benzo[d]imidazole-5-carboxylic acid (Compound 68)

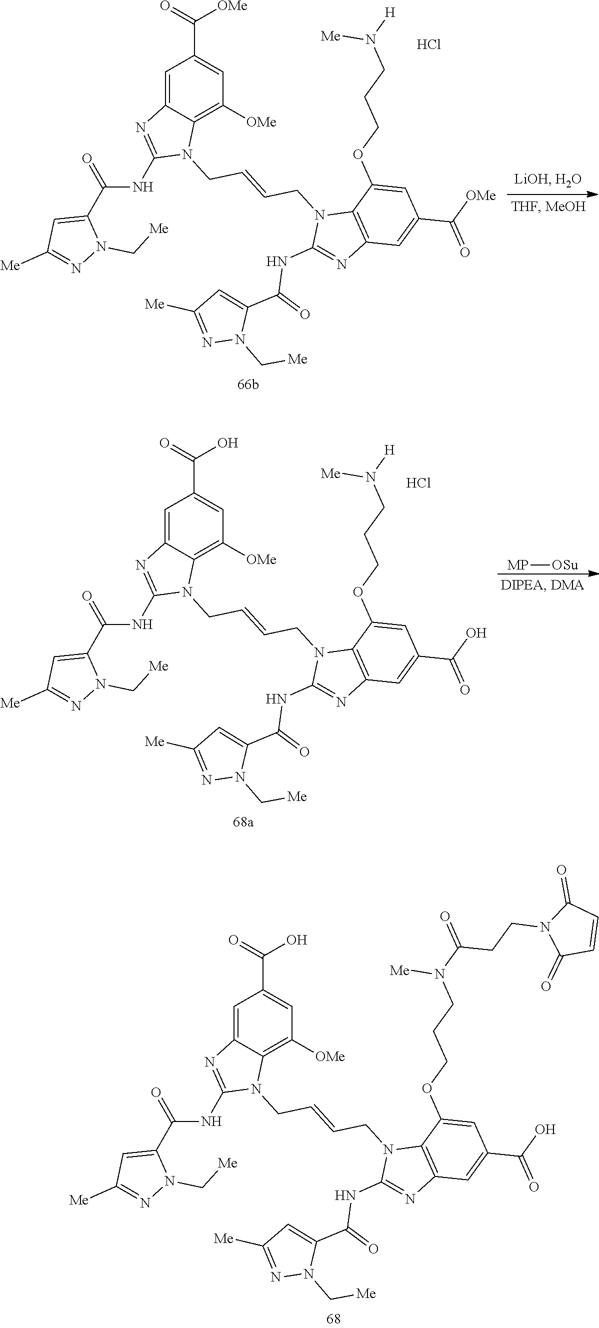

### Synthesis of 68a

Compound 66b (30 mg, 0.032 mmol) was dissolved in methanol (0.32 mL) and 1M LiOH (0.256 mL, 0.256 mmols, 8 equiv.) was added. The mixture was stirred at 80° C. for 1h. The mixture was concentrated in vacuo and purified via prepHPLC (Method H with 0.05% TFA) to afford 68a as a white solid (17.4 mg, 0.0191 mmol, 60% yield). MS (Method D, ESI+): m/z [M+H]⁺=796.4 (theoretical); 796.4 (observed). HPLC retention time: 1.83 min.

### Synthesis of 68

Compound 68a (16.7 mg, 0.0183 mmol, 1 equiv.) and MP-OSu (7.3 mg, 0.0275 mmol, 1.5 equiv.) were dissolved in DMA (0.37 mL) and DIPEA (10 μL, 0.0574 mmol, 2 equiv.) was added. The reaction was stirred at room temperature for 1 hour, AcOH (10 μL) was added and the crude product was purified via prepHPLC (Method H with 0.05% TFA) to afford 68 as a white solid (7.6 mg, 0.0080 mmol, 44% yield). MS (Method D, ESI+): m/z [M+H]⁺=974.4 (theoretical); 974.4 (observed). HPLC retention time: 2.42 min.

### Synthesis of 1-((E)-4-(5-carboxy-2-(1-ethyl-3-methyl-1H-pyrazole-5-carboxamido)-7-methoxy-1H-benzo[d]imidazol-1-yl)but-2-en-1-yl)-7-(3-((((4-(((2S,3R,4S,5S,6S)-6-carboxy-3,4,5-trihydroxytetrahydro-2H-pyran-2-yl)oxy)-2-(3-(3-(2,5-dioxo-2,5-dihydro-1H-pyrrol-1-yl)propanamido)propanamido)benzyl)oxy)carbonyl)(methyl)amino)propoxy)-2-(1-ethyl-3-methyl-1H-pyrazole-5-carboxamido)-1H-benzo[d]imidazole-5-carboxylic acid (Compound 69)

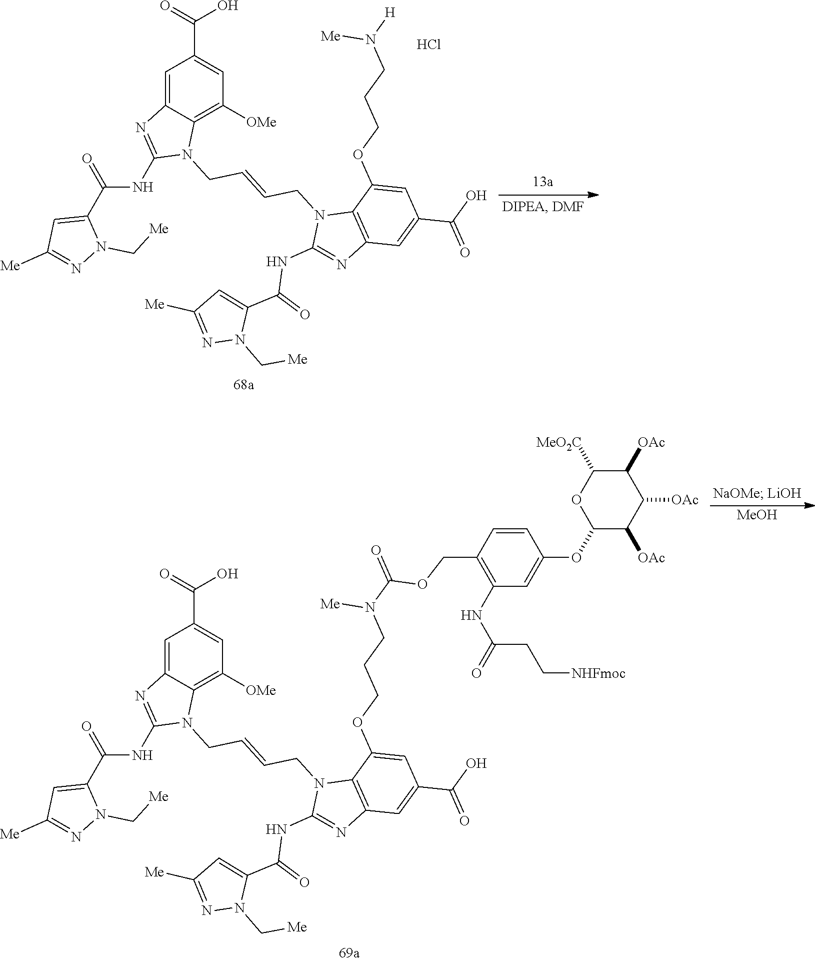

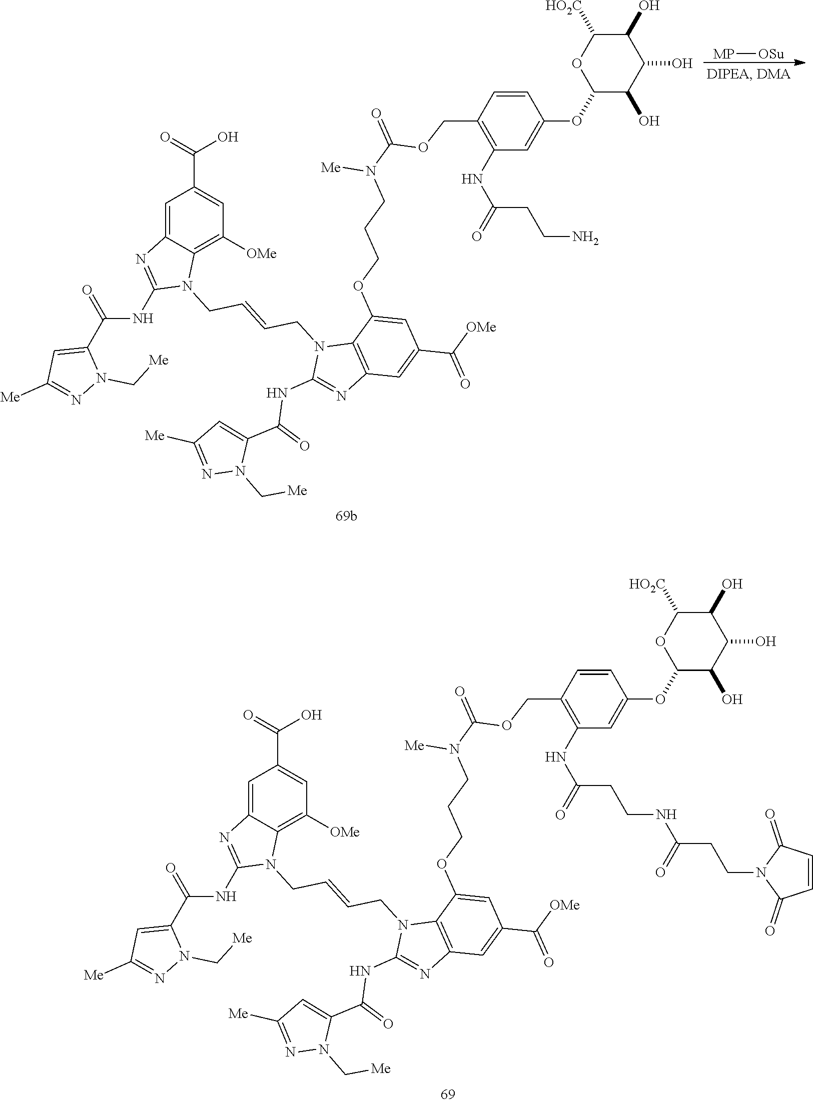

### Synthesis of 69a

Compound 69a was prepared as previously described (see “Synthesis of 67a”). MS (Method E, ESI+): m/z [M+H]⁺=1570.6 (theoretical); 1570.4 (observed). HPLC retention time: 1.95 min.

### Synthesis of 69b

Compound 69b was prepared as previously described (see “Synthesis of 67b”). MS (Method E, ESI+): m/z [M+H]⁺=1208.5 (theoretical); 1208.3 (observed). HPLC retention time: 1.48 min.

### Synthesis of 69

Compound 69 was prepared as previously described (see “Synthesis of 67”). MS (Method E, ESI+): m/z [M+H]⁺=1359.5 (theoretical); 1359.4 (observed). HPLC retention time: 1.68 min.

### Synthesis of (E)-1-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide (Compound 70)

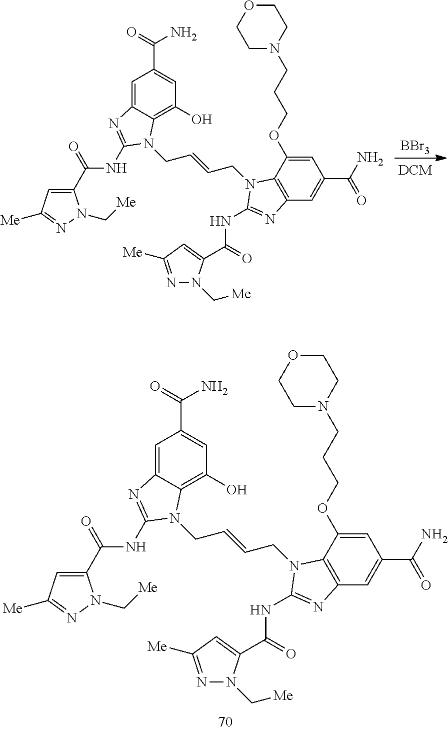 [SMILES: CCn1nc(C)cc1C(=O)Nc1nc2cc(C(N)=O)cc(O)c2n1C/C=C/Cn1c(NC(=O)c2cc(C)nn2CC)nc2cc(C(N)=O)cc(OCCCN3CCOCC3)c21 ; "BBr3" ; CCn1nc(C)cc1C(=O)Nc1nc2cc(C(N)=O)cc(O)c2n1C/C=C/Cn1c(NC(=O)c2cc(C)nn2CC)nc2cc(C(N)=O)cc(OCCCN3CCOCC3)c21]

### Synthesis of 70

To a solution of compound A (6 mg, 0.00706 mmol) in dry DCM (0.10 mL) was added BBr₃ (0.04 mL, 1M in DCM) dropwise. The slurry that formed was stirred overnight at 30° C. under argon. The reaction was monitored by UPLC-MS. Upon completion, cold water (0.10 mL) was added and the mixture was stirred vigorously. After 30 min., the solvent was evaporated, and the product purified by prepHPLC (Method G) using formic acid as the additive. Pure fractions were collected, frozen, and lyophilized to afford compound 70 (5.14 mg, 0.00528 mmol, 75% yield) as a white solid. UPLC-MS (Method D, ESI+): m/z [M+H]⁺=836.9 (theoretical), 836.6 (observed). HPLC retention time: 1.34 min.

The Cysteine Adducts of Compounds 17-24 were Prepared Using the Following Method.

General Method 6. A 10 mM solution of maleimide was incubated with 1 equiv. of L-cysteine (100 mM in water) at 37° C. for 1 hour and the product used without any further purification.

| Compound | Structure | LC-MS data |
|---|---|---|
| 71 | 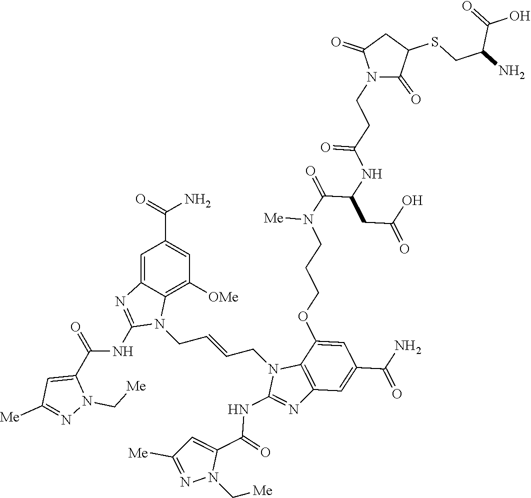 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CC(=O)O)NC(=O)CCN3C(=O)CC(SC[C@H](N)C(=O)O)C3=O)c21] | RT: 1.17 Theoretical: 1181.5 Observed: 1182.1 |
| 72 | 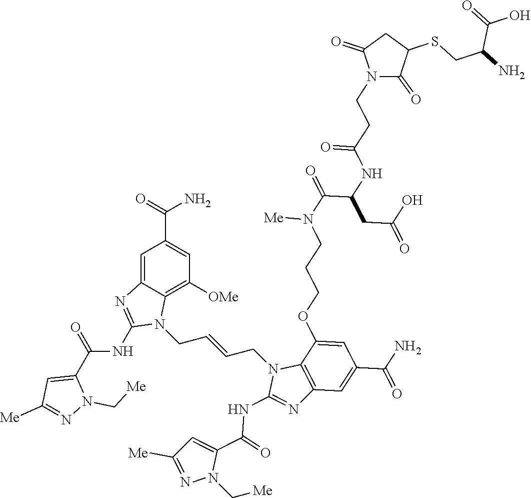 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CC(=O)O)NC(=O)CCN3C(=O)CC(SC[C@H](N)C(=O)O)C3=O)c21] | RT: 1.21 Theoretical: 1195.5 Observed: 1195.7 |
| 73 | 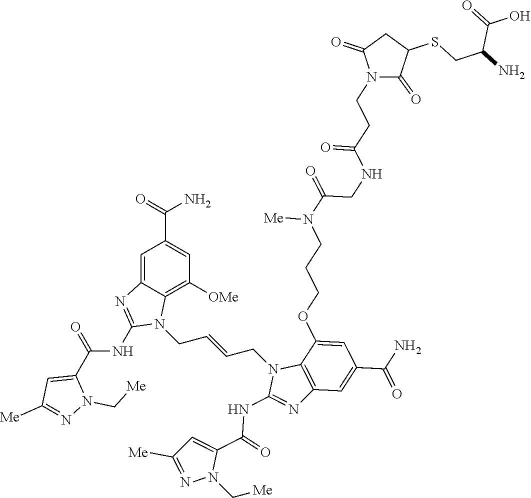 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)CNC(=O)CCN3C(=O)CC(SC[C@H](N)C(=O)O)C3=O)c21] | RT: 1.17 Theoretical: 1123.5 Observed: 1124.0 |
| 74 | 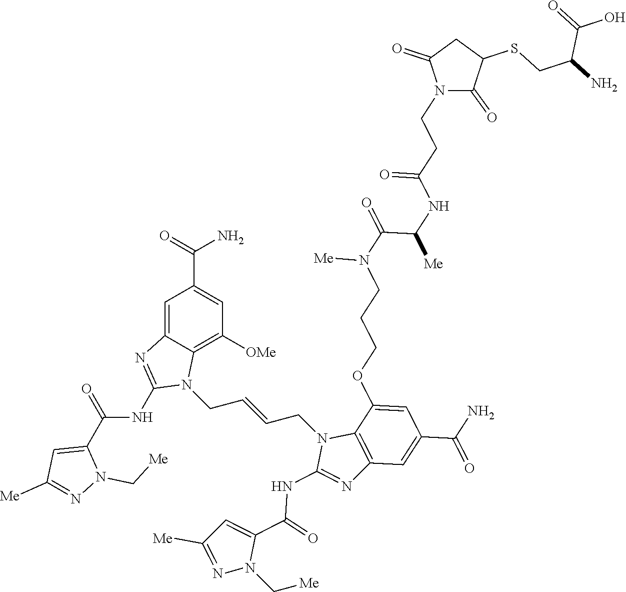 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](C)NC(=O)CCN3C(=O)CC(SC[C@H](N)C(=O)O)C3=O)c21] | RT: 1.19 Theoretical: 1137.5 Observed: 1137.9 |
| 75 | 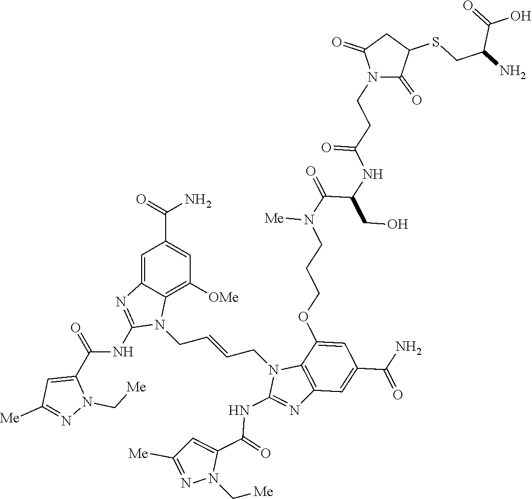 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CO)NC(=O)CCN3C(=O)CC(SC[C@H](N)C(=O)O)C3=O)c21] | RT: 1.16 Theoretical: 1153.5 Observed: 1153.8 |
| 76 | 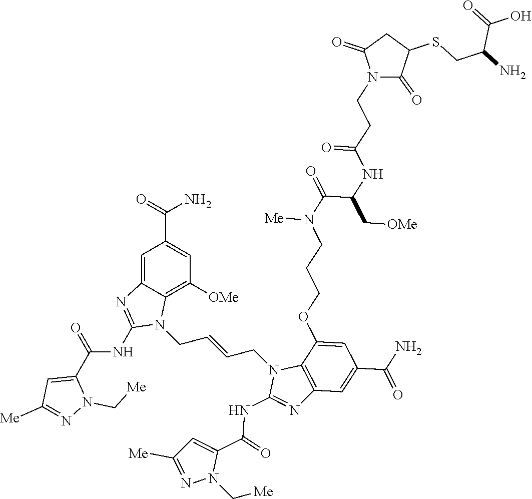 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](COC)NC(=O)CCN3C(=O)CC(SC[C@H](N)C(=O)O)C3=O)c21] | RT: 1.19 Theoretical: 1167.5 Observed: 1168.0 |

### Synthesis of tert-butyl (3-(5-carbamoyl-2-chloro-3-nitrophenoxy)propyl)(methyl)carbamate (Compound 77)

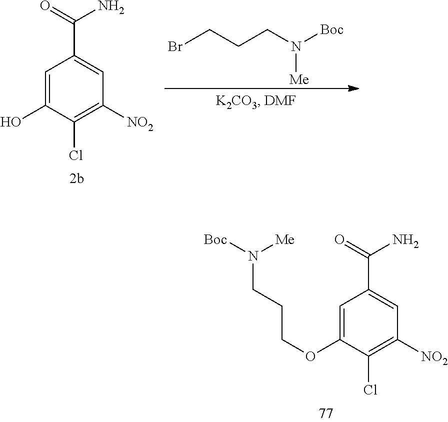 [SMILES: NC(=O)c1cc(O)c(Cl)c([N+](=O)[O-])c1 ; CN(CCCBr)C(=O)OC(C)(C)C ; CN(CCCOc1cc(C(N)=O)cc([N+](=O)[O-])c1Cl)C(=O)OC(C)(C)C]

A flame-dried 100 mL RB with stir bar was charged with a solution of 2b (1.0 g, 4.62 mmol, 1 equiv.) in DMF (10 mL), potassium carbonate (830 mg, 6.00 mmol, 1.3 equiv.) and a solution of tert-butyl N-(3-bromopropyl)-N-methyl-carbamate (1.20 eq, 1.40 g, 5.54 mmol, 1.20 equiv.) in DMF (5 mL). Additional DMF was added to bring the total volume to 45 mL and the reaction was heated to 70° C. for 24 hours. The reaction was cooled to room temperature and filtered over celite washing with DMF (3×10 mL). This solution was poured into ice water (900 mL), stirred for 90 minutes and crude product isolated via filtration. Finally, the filtrate was washed with cold water (300 mL) and dried in vacuo overnight to give 77 (1.23 g, 3.16 mmol, 68% yield).

### Synthesis of tert-butyl (E)-(3-((2-amino-5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-7-yl)oxy)propyl)(methyl)carbamate (Compound 78)

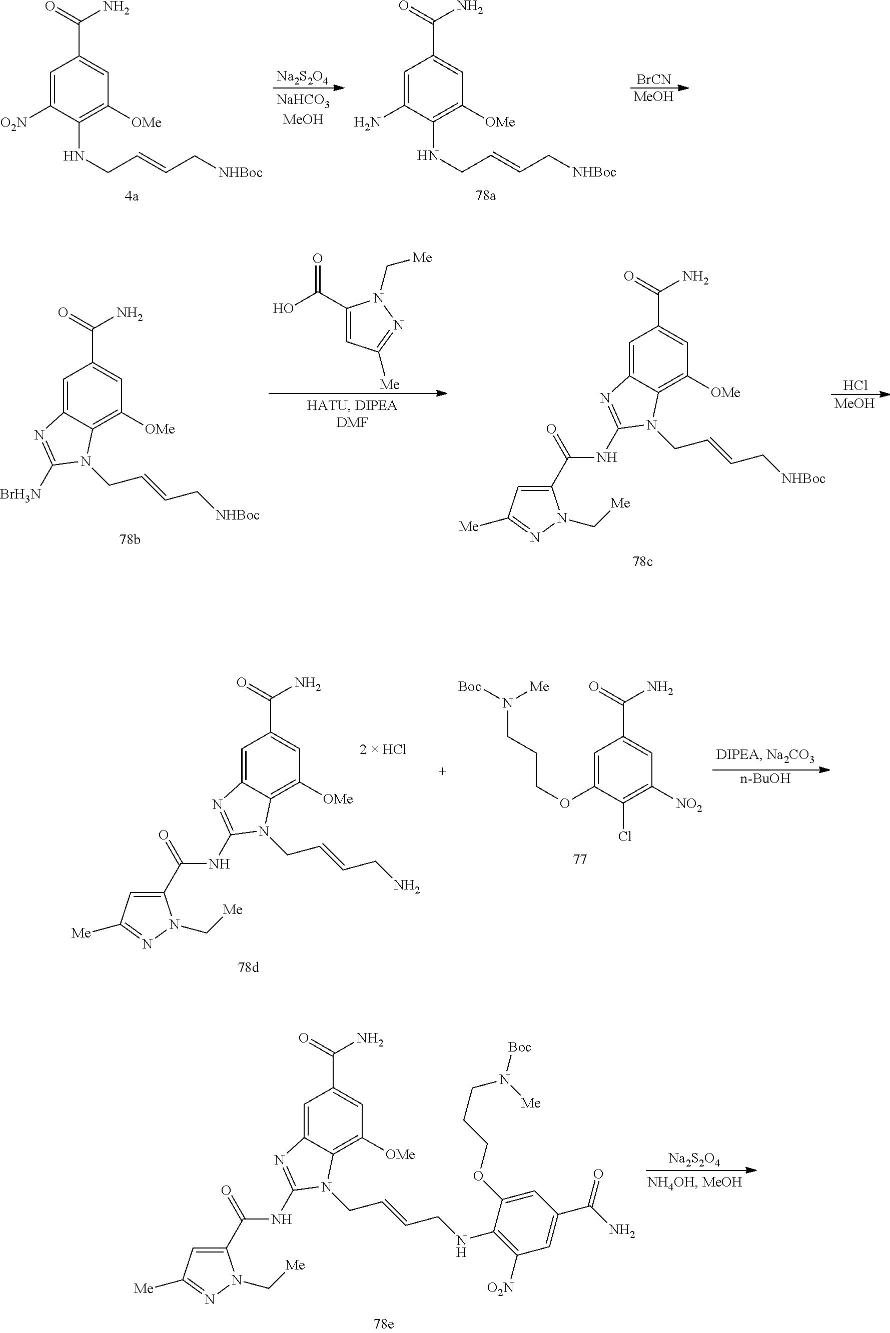

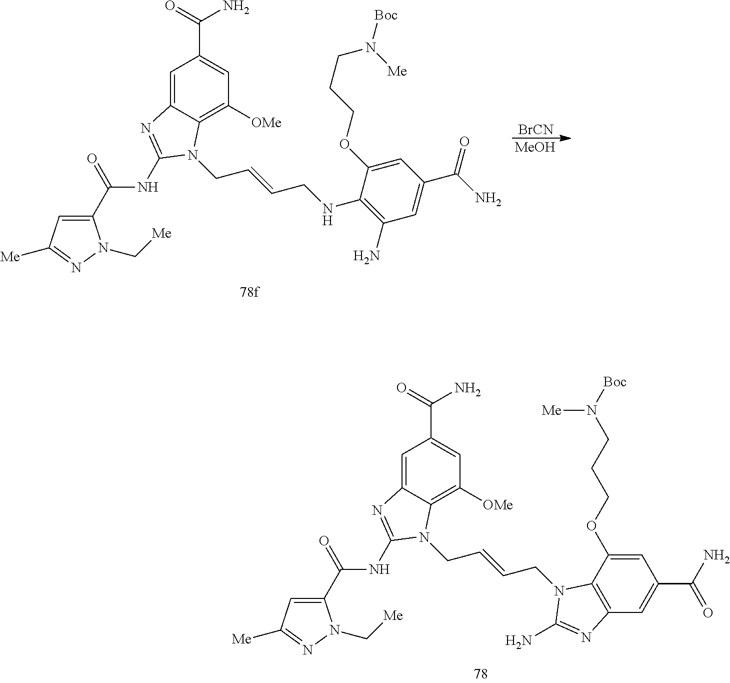

### Synthesis of 78a

A 500 mL round bottom flask with stir bar was charged with 4a (3.0 g, 7.9 mmol, 1 equiv.) and sodium bicarbonate (12.5 g, 148 mmol, 19 equiv.) and ethanol (105 mL) was added to give a heterogenous solution. This solution was cooled in an ice-bath and a solution of sodium hydrosulfite (25.8 g, 148 mmol, 19 equiv.) in 105 mL water was added dropwise at such a rate to keep the internal temperature below 10° C. The mixture was heated open to the atmosphere to 45° C. for 1 hour and cooled to room temperature. The mixture was filtered over celite, washing with EtOH (100 mL) and solvent removed in vacuo. The crude material was redissolved in 1:1 DCM:MeOH (200 mL), filtered over celite and solvent removed in vacuo. This procedure was repeated once more and then the crude product was loaded onto celite and purified by flash chromatography (50g Sfar HC Duo, SiO₂ column, 0-40% 10:1 NH₄OH:MeOH in DCM) to give 78a (1.45 g, 4.13 mmol, 52% yield). MS (Method D, ESI+): m/z [M+H]⁺=351.2 (theoretical); 351.1 (observed). HPLC retention time: 1.53 min.

### Synthesis of 78b

An oven-dried 200 mL round bottom flask was charged with 78a (1.95 g, 5.58 mmol, 1 equiv.) which was dissolved in methanol (45 mL) and cyanogen bromide (3M in DCM, 5.6 mL, 16.7 mmol, 3 equiv.) was added to give a yellow homogenous solution. The reaction was stirred at room temperature for 48 hours and solvent removed in vacuo to give 78b as the HBr salt (2.48 g, 5.44 mmol, 98% yield). MS (Method D, ESI+): m/z [M+H]⁺=376.2 (theoretical); 376.1 (observed). HPLC retention time: 0.71 min.

### Synthesis of 78c

A flame dried 40 mL vial with stir bar was charged with 78b HBr (867 mg, 1.90 mmol, 1 equiv.), 2-ethyl-5-methyl-pyrazole-3-carboxylic acid (879 mg, 5.70 mmol, 3 equiv.), and HATU (2.17 g, 5.70 mmol, 3 equiv.). The solids were dissolved in DMF (15 mL) and then DIPEA (2.0 mL, 11.4 mmol, 6 equiv.) was added. The vial was sealed and stirred at room temperature for 48 hours. The solution was poured into ice-cold water (450 mL) with NH₄OH (28% NH₃ in water, 10 mL) and allowed to precipitate at 4° C. overnight. The white precipitate was isolated via filtration and dried in vacuo overnight to give 78c (658 mg, 1.29 mmol, 68% yield). MS (Method D, ESI+): m/z [M+H]⁺=512.3 (theoretical); 512.2 (observed). HPLC retention time: 2.35 min.

### Synthesis of 78d

An oven dried 8 mL vial with stir bar was charged with 78c (800 mg, 1.56 mmol, 3 equiv.) which was stirred in 3M HCl in MeOH (5.2 mL, 15.6 mmol HCl, 10 equiv.) for 1 hour. The solvent removed in vacuo to give 78d as the 2×HCl salt (700 mg, 1.56 mmol, quantitative yield). MS (Method D, ESI+): m/z [M+H]⁺=412.2 (theoretical); 412.5 (observed). HPLC retention time: 0.73 min.

### Synthesis of 78e

An oven-dried 20 mL microwave vial was charged with 78e (700 mg, 1.56 mmol, 1 equiv.), 77 (909 mg, 2.34 mmol, 1.5 equiv.) and sodium carbonate (497 mg, 4.69 mmol, 3 equiv.) and to the mixture was added 1-butanol (15 mL) and DIPEA (1.6 mL, 9.38 mmol, 6 equiv.). The vial was sealed and heated in a microwave reactor at 140° C. for 3 hours to give a bright red heterogenous mixture. This mixture was poured into DCM (100 mL) and filtered over celite washing with DCM (50 mL) and MeOH (50 mL). The crude product was loaded onto celite and purified via flash chromatography (50g Sfar HC Duo, SiO₂ column, 0-20% MeOH in DCM) to give 78e (569 mg, 0.746 mmol, 48% yield) as a bright red solid. MS (Method D, ESI+): m/z [M+H]⁺=763.4 (theoretical); 763.4 (observed). HPLC retention time: 2.17 min.

### Synthesis of 78f

To a mixture of 78e (569 mg, 0.746 mmol, 1 equiv.) in methanol (8 mL) and NH₄OH (2.0 mL, 28% NH₃ in water) was added a solution of sodium hydrosulfite (2.34 g, 13.4 mmol, 18 equiv.) in water (8 mL). This solution was heated at 50° C. for 1 hour. The reaction was poured into a separatory funnel containing water (250 mL) and EtOAc (250 mL). The mixture was shaken, layers separated and the aqueous layer was further extracted with EtOAc (3×100 mL). The organics were combined, washed with brine (2×100 mL), dried with MgSO₄, filtered and solvent removed in vacuo to give 78f (400 mg, 0.546 mmol, 73% yield) as a tan solid. MS (Method D, ESI+): m/z [M+H]⁺=733.4 (theoretical); 733.6 (observed). HPLC retention time: 1.39 min.

### Synthesis of 78

To a solution of 78f (1.00 eq, 400 mg, 0.546 mmol) in methanol (5.5 mL) was added cyanogen bromide (3M in DCM, 0.55 mL, 1.65 mmol, 3 equiv.) and the mixture was stirred at room temperature for 24 hours. The solvent was removed in vacuo to give 78 as the HBr salt (456 mg, 0.544 mmol, quantitative yield). MS (Method D, ESI+): m/z [M+H]⁺=758.4 (theoretical); 758.6 (observed). HPLC retention time: 1.19 min.

Library Synthesis of Amide Analogs, Group #2. Scheme and General Methods. Compounds 71-95.

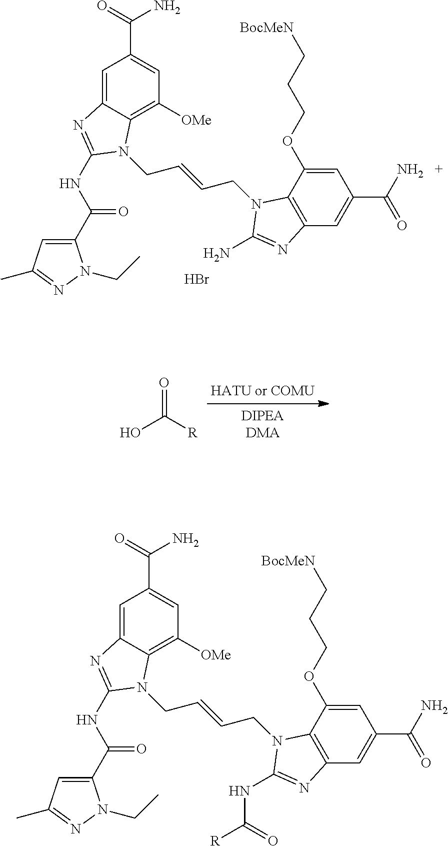

COMU Couplings (General Method 7A): A 2 mL microwave vial was charged with a solution of compound 78 (20 mg, 0.0238 mmol, 1 equiv.) in DMA (0.50 mL). The respective carboxylic acid (2 equiv.), COMU (20.4 mg, 0.0477 mmol, 2 equiv.) and DIPEA (20.8 μL, 0.119 mmol, 5 equiv.) were added. The vial was sealed and heated to 80° C. in a microwave reactor for 1h. The reaction was monitored via UPLC-MS (Method E, ESI+). Upon completion, acetic acid (20 μL) was added and the resulting product was purified by prepHPLC (Method H) using 0.05% TFA as the additive. Pure fractions were collected, frozen, and lyophilized to afford product as a white solid.

HATU Couplings (General Method 7B): A 2 mL microwave vial was charged with a solution of compound 78 (20 mg, 0.0238 mmol, 1 equiv.) in DMA (0.50 mL). The respective carboxylic acid (4 equiv.), HATU (36.3 mg, 0.0954 mmol, 2 equiv.) and DIPEA (20.8 μL, 0.119 mmol, 5 equiv.) were added. The vial was sealed and heated to 80° C. a microwave reactor for 1h. The reaction was monitored via UPLC-MS (Method E, ESI+). Upon completion, acetic acid (20 μL) was added and the resulting product was purified by prepHPLC (Method H) using 0.05% TFA as the additive. Pure fractions were collected, frozen, and lyophilized to afford product as a white solid.

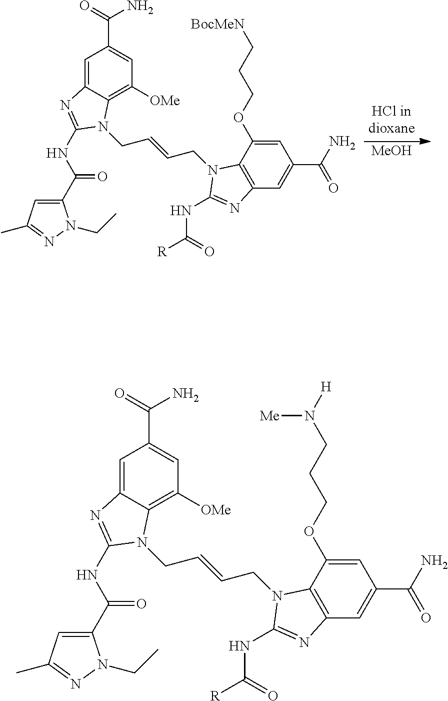

Boc Deprotection (General Method 8): The resulting product general method 7A or 7B was dissolved in MeOH (0.01 M), to which 4M HCl in dioxane (8 equiv.) was added. The solution stirred at room temperature for 30 min. The reaction was monitored via UPLC-MS (Method E, ESI+). Upon completion, the solution was concentrated, redissolved in DMSO, and purified via prepHPLC (Method G or H) using TFA as the additive. Pure fractions were collected, frozen, and lyophilized to afford product as a white solid.

| | | | | | Yield |
|---|---|---|---|---|---|
| | | | Boc LC- | Amine LC- | (over 2 |
| Cmpd. | Structure | Method | MS data | MS data | steps) |
| 79 | 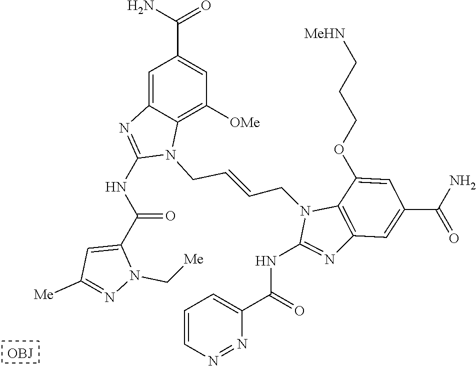 [SMILES: CCn1nc(C)cc1C(=O)Nc1nc2cc(C(N)=O)cc(OC)c2n1C/C=C/Cn1c(NC(=O)c2cccnn2)nc2cc(C(N)=O)cc(OCCCNC)c21] | 7A | RT: 1.57 Theoretical: 864.4 Observed: 864.7 | RT: 1.32 Theoretical: 764.3 Observed: 764.4 | 17% 4.5 mg 0.00406 mmol |
| 80 | 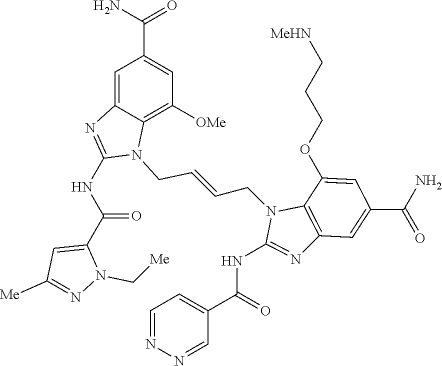 [SMILES: CCn1nc(C)cc1C(=O)Nc1nc2cc(C(N)=O)cc(OC)c2n1C/C=C/Cn1c(NC(=O)c2ccnnc2)nc2cc(C(N)=O)cc(OCCCNC)c21] | 7A | RT: 2.18 Theoretical: 864.4 Observed: 864.4 | RT: 1.24 Theoretical: 764.3 Observed: 764.4 | 19% 5.0 mg 0.00455 mmol |
| 81 | 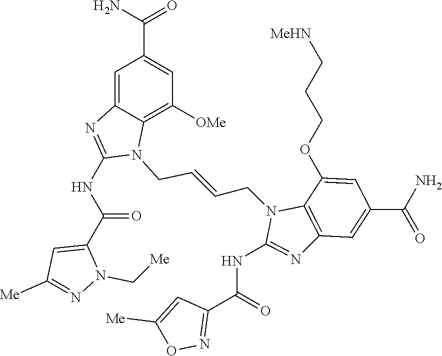 [SMILES: CCn1nc(C)cc1C(=O)Nc1nc2cc(C(N)=O)cc(OC)c2n1C/C=C/Cn1c(NC(=O)c2cc(C)on2)nc2cc(C(N)=O)cc(OCCCNC)c21] | 7A | RT: 1.79 Theoretical: 867.4 Observed: 867.7 | RT: 1.55 Theoretical: 767.3 Observed: 767.3 | 18% 4.8 mg 0.00436 mmol |
| 82 | 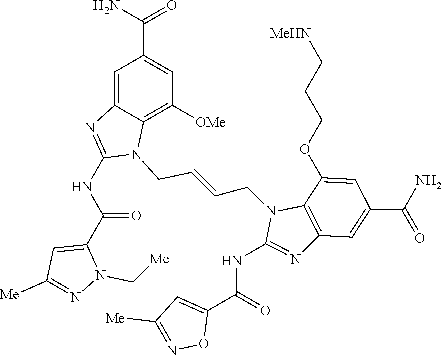 [SMILES: CCn1nc(C)cc1C(=O)Nc1nc2cc(C(N)=O)cc(OC)c2n1C/C=C/Cn1c(NC(=O)c2cc(C)no2)nc2cc(C(N)=O)cc(OCCCNC)c21] | 7A | RT: 1.83 Theoretical: 867.4 Observed: 967.4 | RT: 1.37 Theoretical 767.3 Observed: 767.3 | 9% 2.4 mg 0.00216 mmol |
| 83 | 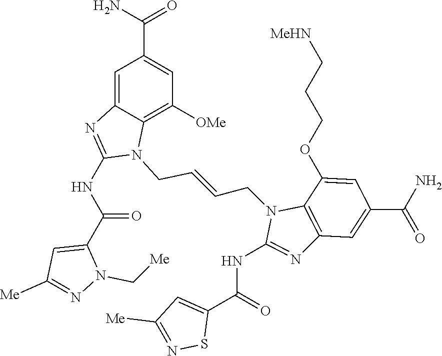 [SMILES: CCn1nc(C)cc1C(=O)Nc1nc2cc(C(N)=O)cc(OC)c2n1C/C=C/Cn1c(NC(=O)c2cc(C)ns2)nc2cc(C(N)=O)cc(OCCCNC)c21] | 7B | RT: 1.85 Theoretical: 883.4 Observed: 883.4 | RT: 1.35 Theoretical: 783.3 Observed: 783.3 | 10% 2.8 mg 0.00247 mmol |
| 84 | 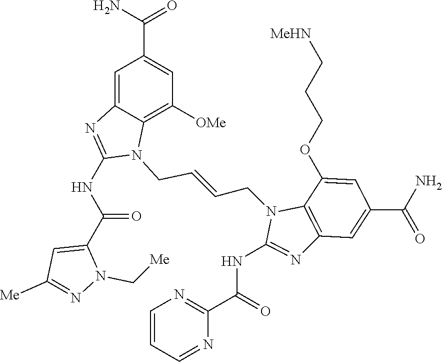 [SMILES: CCn1nc(C)cc1C(=O)Nc1nc2cc(C(N)=O)cc(OC)c2n1C/C=C/Cn1c(NC(=O)c2ncccn2)nc2cc(C(N)=O)cc(OCCCNC)c21] | 7A | RT: 1.69 Theoretical: 864.4 Observed: 864.4 | RT: 1.18 Theoretical: 764.3 Observed: 764.4 | 5% 1.2 mg 0.00110 mmol |
| 85 | 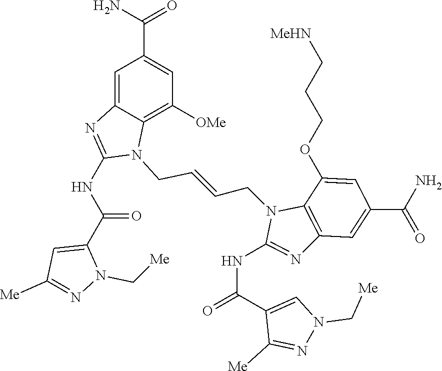 [SMILES: CCn1cc(C(=O)Nc2nc3cc(C(N)=O)cc(OCCCNC)c3n2C/C=C/Cn2c(NC(=O)c3cc(C)nn3CC)nc3cc(C(N)=O)cc(OC)c32)c(C)n1] | 7B | RT: 1.75 Theoretical: 894.4 Observed: 894.5 | RT: 1.31 Theoretical: 794.4 Observed: 794.4 | 3% 0.88 mg 0.00077 mmol |
| 86 | 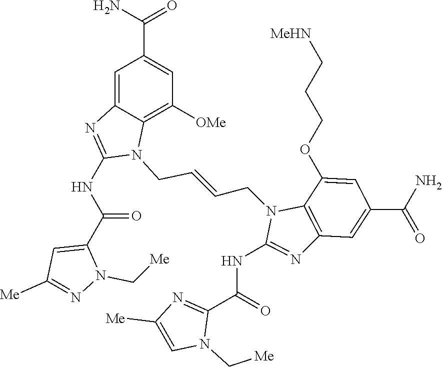 [SMILES: CCn1cc(C)nc1C(=O)Nc1nc2cc(C(N)=O)cc(OCCCNC)c2n1C/C=C/Cn1c(NC(=O)c2cc(C)nn2CC)nc2cc(C(N)=O)cc(OC)c21] | 7B | RT: 1.59 Theoretical: 894.4 Observed: 894.5 | RT: 1.19 Theoretical: 794.4 Observed: 794.4 | 11% 3.1 mg 0.00270 mmol |
| 87 | 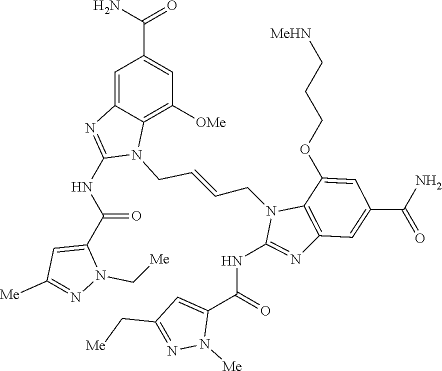 [SMILES: CCc1cc(C(=O)Nc2nc3cc(C(N)=O)cc(OCCCNC)c3n2C/C=C/Cn2c(NC(=O)c3cc(C)nn3CC)nc3cc(C(N)=O)cc(OC)c32)n(C)n1] | 7B | RT: 1.70 Theoretical: 894.4 Observed: 894.5 | RT: 1.38 Theoretical: 794.4 Observed: 794.4 | 13% 3.5 mg 0.00307 mmol |
| 88 | 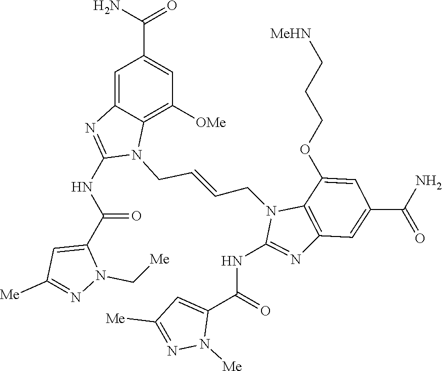 [SMILES: CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCCNC)c21] | 7B | RT: 1.63 Theoretical: 880.4 Observed: 880.5 | RT: 1.28 Theoretical: 780.4 Observed: 780.4 | 16% 4.4 mg 0.00391 mmol |
| 89 | 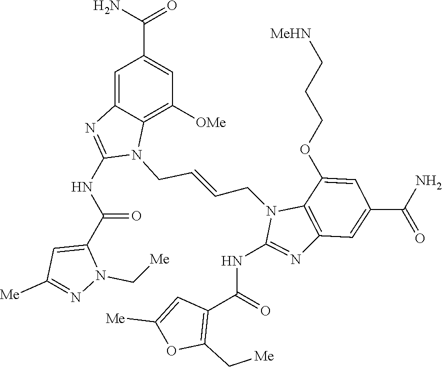 [SMILES: CCc1oc(C)cc1C(=O)Nc1nc2cc(C(N)=O)cc(OCCCNC)c2n1C/C=C/Cn1c(NC(=O)c2cc(C)nn2CC)nc2cc(C(N)=O)cc(OC)c21] | 7B | RT: 1.77 Theoretical: 894.4 Observed: 894.5 | RT: 1.46 Theoretical: 794.4 Observed: 794.4 | 9% 2.4 mg 0.00211 mmol |
| 90 | 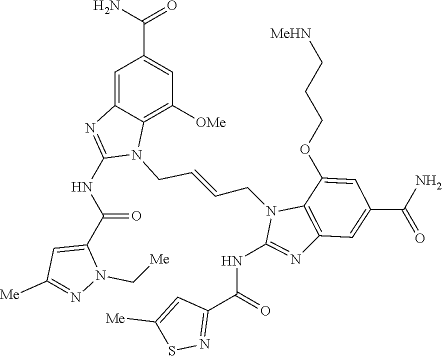 [SMILES: CCn1nc(C)cc1C(=O)Nc1nc2cc(C(N)=O)cc(OC)c2n1C/C=C/Cn1c(NC(=O)c2cc(C)sn2)nc2cc(C(N)=O)cc(OCCCNC)c21] | 7B | RT: 1.70 Theoretical: 883.4 Observed: 883.4 | RT: 1.30 Theoretical: 783.3 Observed: 783.4 | 17% 4.7 mg 0.00416 mmol |
| 91 | 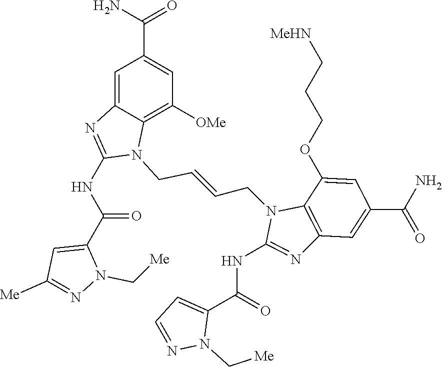 [SMILES: CCn1nc(C)cc1C(=O)Nc1nc2cc(C(N)=O)cc(OC)c2n1C/C=C/Cn1c(NC(=O)c2ccnn2CC)nc2cc(C(N)=O)cc(OCCCNC)c21] | 7B | RT: 1.65 Theoretical: 880.4 Observed: 880.5 | RT: 1.32 Theoretical: 780.4 Observed: 780.4 | 6% 1.7 mg 0.00153 mmol |
| 92 | 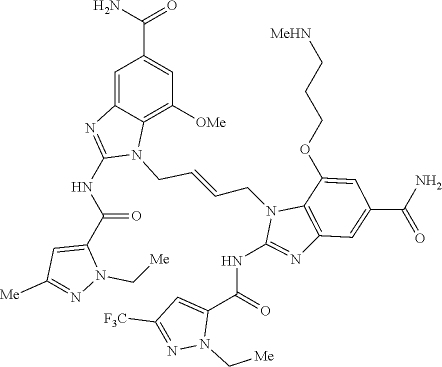 [SMILES: CCn1nc(C)cc1C(=O)Nc1nc2cc(C(N)=O)cc(OC)c2n1C/C=C/Cn1c(NC(=O)c2cc(C(F)(F)F)nn2CC)nc2cc(C(N)=O)cc(OCCCNC)c21] | 7B | RT: 1.83 Theoretical: 948.4 Observed: 948.5 | RT: 1.49 Theoretical: 848.4 Observed: 848.4 | 10% 2.7 mg 0.00229 mmol |
| 93 | 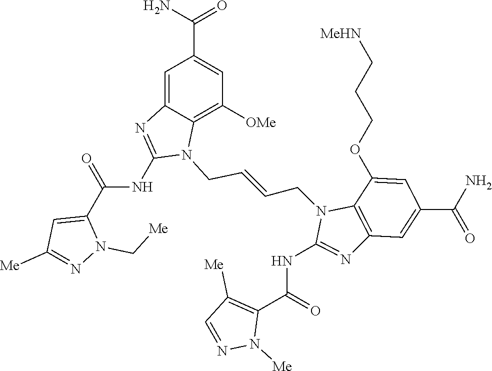 [SMILES: CCn1nc(C)cc1C(=O)Nc1nc2cc(C(N)=O)cc(OC)c2n1C/C=C/Cn1c(NC(=O)c2c(C)cnn2C)nc2cc(C(N)=O)cc(OCCCNC)c21] | 7B | RT: 1.74 Theoretical: 880.4 Observed: 880.5 | RT: 1.37 Theoretical: 780.4 Observed: 780.4 | 22% 5.9 mg 0.00529 mmol |
| 94 | 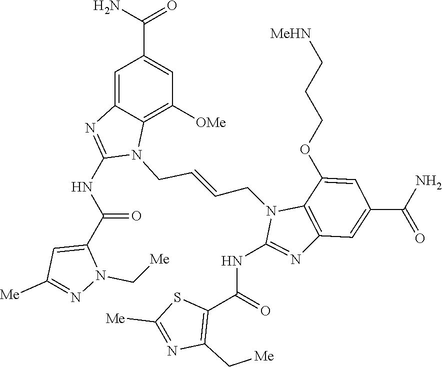 [SMILES: CCc1nc(C)sc1C(=O)Nc1nc2cc(C(N)=O)cc(OCCCNC)c2n1C/C=C/Cn1c(NC(=O)c2cc(C)nn2CC)nc2cc(C(N)=O)cc(OC)c21] | 7B | RT: 1.88 Theoretical: 911.4 Observed: 911.5 | RT: 1.38 Theoretical: 811.3 Observed: 811.4 | 30% 8.1 mg 0.00703 mmol |
| 95 | 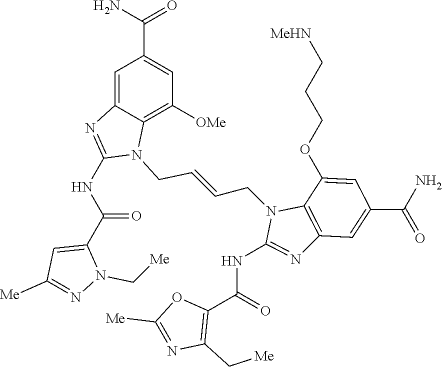 [SMILES: CCc1nc(C)oc1C(=O)Nc1nc2cc(C(N)=O)cc(OCCCNC)c2n1C/C=C/Cn1c(NC(=O)c2cc(C)nn2CC)nc2cc(C(N)=O)cc(OC)c21] | 7B | RT: 1.86 Theoretical: 895.4 Observed: 895.5 | RT: 1.41 Theoretical: 795.4 Observed: 795.4 | 8% 2.1 mg 0.0018 mmol |

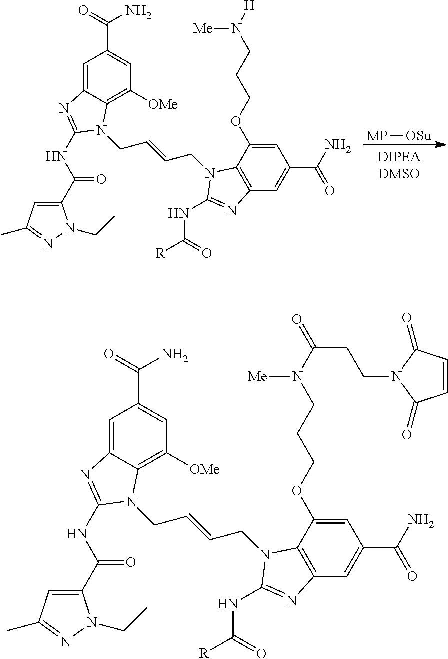

Maleimide Couplings (General Method 9): The resulting amine from the previous reaction (compounds 79-95) was dissolved in DMSO (0.01M), to which was added MP-OSu (2 equiv.) and DIPEA (5 equiv.). The mixture was stirred at 30° C. overnight, and monitored by UPLC-MS (Method E, ESI+). Upon completion, the resulting product was purified via prepHPLC (Method G) using 0.05% TFA as the additive.

| | | LC-MS data | |
|---|---|---|---|
| Cmpd. | Structure | [M + H]⁺ | Yield |
| 96 | 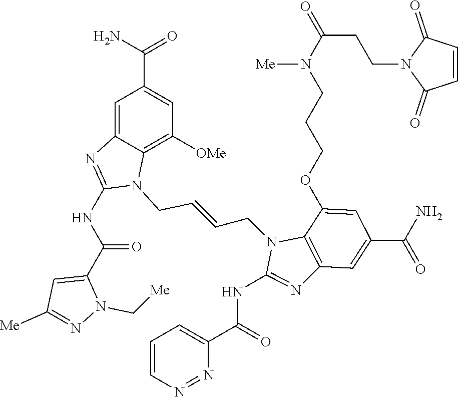 [SMILES: CCn1nc(C)cc1C(=O)Nc1nc2cc(C(N)=O)cc(OC)c2n1C/C=C/Cn1c(NC(=O)c2cccnn2)nc2cc(C(N)=O)cc(OCCCN(C)C(=O)CCN3C(=O)C=CC3=O)c21] | RT: 1.38 Theoretical: 915.4 Observed: 915.4 | 68% 3.14 mg 0.0027 mmol |
| 97 | 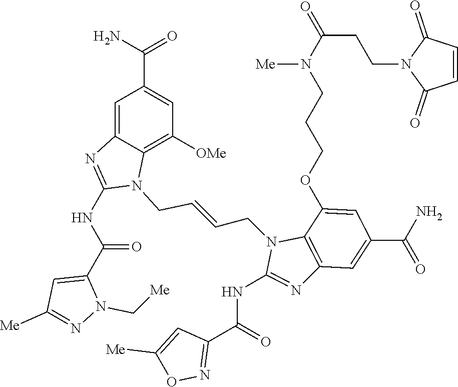 [SMILES: CCn1nc(C)cc1C(=O)Nc1nc2cc(C(N)=O)cc(OC)c2n1C/C=C/Cn1c(NC(=O)c2cc(C)on2)nc2cc(C(N)=O)cc(OCCCN(C)C(=O)CCN3C(=O)C=CC3=O)c21] | RT: 1.65 Theoretical: 918.4 Observed: 918.4 | 62% 3.11 mg 0.0027 mmol |
| 98 | 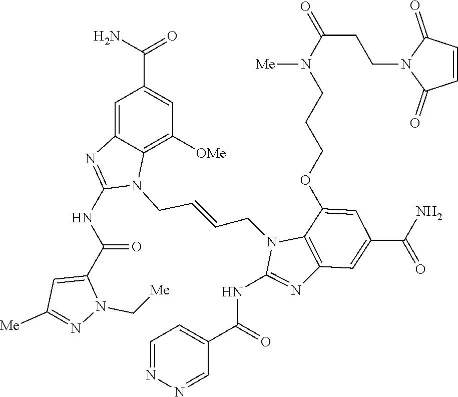 [SMILES: CCn1nc(C)cc1C(=O)Nc1nc2cc(C(N)=O)cc(OC)c2n1C/C=C/Cn1c(NC(=O)c2ccnnc2)nc2cc(C(N)=O)cc(OCCCN(C)C(=O)CCN3C(=O)C=CC3=O)c21] | RT: 1.42 Theoretical: 915.4 Observed: 915.4 | 84% 4.38 mg 0.0038 mmol |
| 99 | 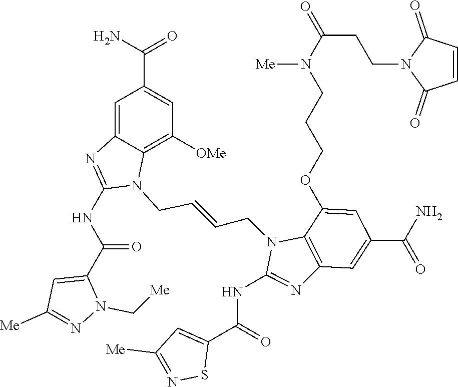 [SMILES: CCn1nc(C)cc1C(=O)Nc1nc2cc(C(N)=O)cc(OC)c2n1C/C=C/Cn1c(NC(=O)c2cc(C)ns2)nc2cc(C(N)=O)cc(OCCCN(C)C(=O)CCN3C(=O)C=CC3=O)c21] | RT: 1.47 Theoretical: 934.3 Observed: 934.4 | 31% 0.89 mg 0.0008 mmol |
| 100 | 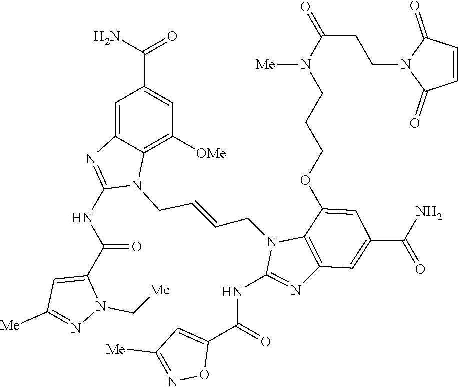 [SMILES: CCn1nc(C)cc1C(=O)Nc1nc2cc(C(N)=O)cc(OC)c2n1C/C=C/Cn1c(NC(=O)c2cc(C)no2)nc2cc(C(N)=O)cc(OCCCN(C)C(=O)CCN3C(=O)C=CC3=O)c21] | RT: 1.50 Theoretical: 918.4 Observed: 918.4 | 53% 1.3 mg 0.0011 mmol |
| 101 | 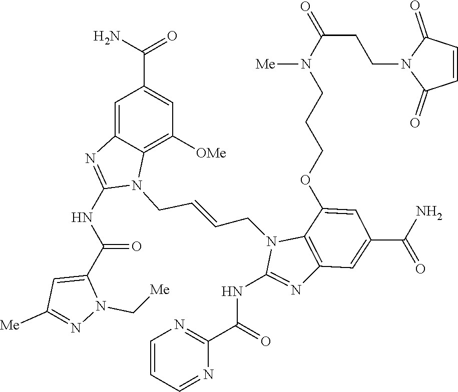 [SMILES: CCn1nc(C)cc1C(=O)Nc1nc2cc(C(N)=O)cc(OC)c2n1C/C=C/Cn1c(NC(=O)c2ncccn2)nc2cc(C(N)=O)cc(OCCCN(C)C(=O)CCN3C(=O)C=CC3=O)c21] | RT: 1.35 Theoretical: 915.4 Observed: 915.4 | 86% 5.88 mg 0.0051 mmol |
| 102 | 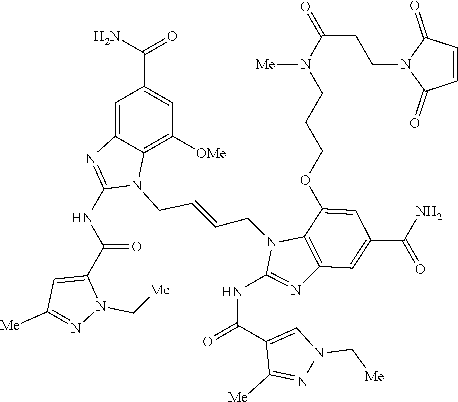 [SMILES: CCn1cc(C(=O)Nc2nc3cc(C(N)=O)cc(OCCCN(C)C(=O)CCN4C(=O)C=CC4=O)c3n2C/C=C/Cn2c(NC(=O)c3cc(C)nn3CC)nc3cc(C(N)=O)cc(OC)c32)c(C)n1] | RT: 1.45 Theoretical: 945.4 Observed: 945.4 | 40% 0.36 mg 0.0003 mmol |
| 103 | 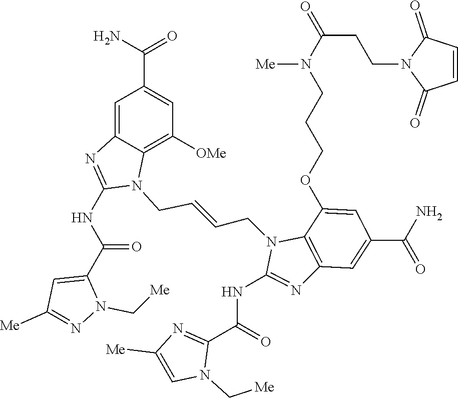 [SMILES: CCn1cc(C)nc1C(=O)Nc1nc2cc(C(N)=O)cc(OCCCN(C)C(=O)CCN3C(=O)C=CC3=O)c2n1C/C=C/Cn1c(NC(=O)c2cc(C)nn2CC)nc2cc(C(N)=O)cc(OC)c21] | RT: 1.37 Theoretical: 945.4 Observed: 945.4 | 41% 1.29 mg 0.0011 mmol |
| 104 | 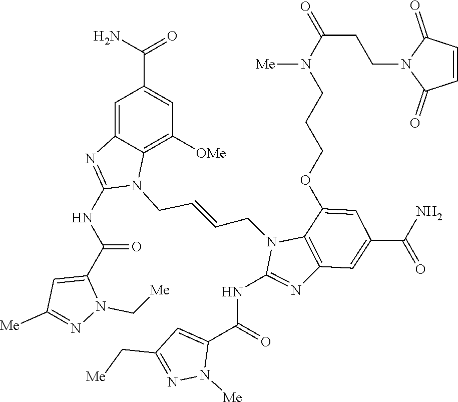 [SMILES: CCc1cc(C(=O)Nc2nc3cc(C(N)=O)cc(OCCCN(C)C(=O)CCN4C(=O)C=CC4=O)c3n2C/C=C/Cn2c(NC(=O)c3cc(C)nn3CC)nc3cc(C(N)=O)cc(OC)c32)n(C)n1] | RT: 1.67 Theoretical: 945.4 Observed: 945.5 | 52% 1.87 mg 0.0016 mmol |
| 105 | 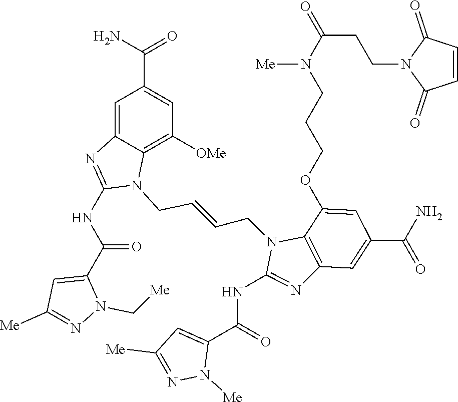 [SMILES: CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCCN(C)C(=O)CCN3C(=O)C=CC3=O)c21] | RT: 1.48 Theoretical: 931.4 Observed: 931.4 | 88% 4.00 mg 0.0035 mmol |
| 106 | 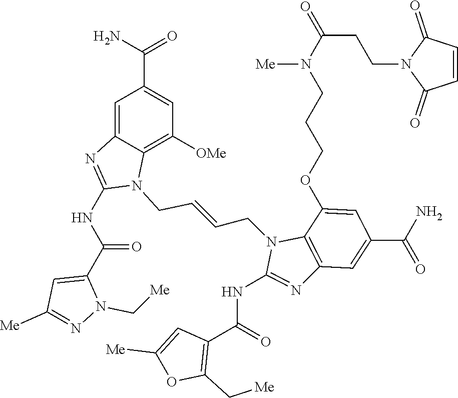 [SMILES: CCc1oc(C)cc1C(=O)Nc1nc2cc(C(N)=O)cc(OCCCN(C)C(=O)CCN3C(=O)C=CC3=O)c2n1C/C=C/Cn1c(NC(=O)c2cc(C)nn2CC)nc2cc(C(N)=O)cc(OC)c21] | RT: 1.61 Theoretical: 945.4 Observed: 945.4 | 31% 0.76 mg 0.0006 mmol |
| 107 | 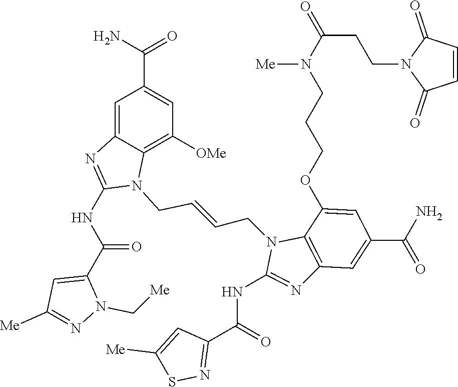 [SMILES: CCn1nc(C)cc1C(=O)Nc1nc2cc(C(N)=O)cc(OC)c2n1C/C=C/Cn1c(NC(=O)c2cc(C)sn2)nc2cc(C(N)=O)cc(OCCCN(C)C(=O)CCN3C(=O)C=CC3=O)c21] | RT: 1.45 Theoretical: 934.3 Observed: 934.4 | 51% 2.46 mg 0.0021 mmol |
| 108 | 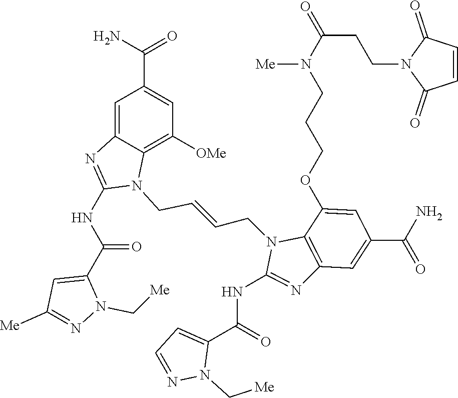 [SMILES: CCn1nc(C)cc1C(=O)Nc1nc2cc(C(N)=O)cc(OC)c2n1C/C=C/Cn1c(NC(=O)c2ccnn2CC)nc2cc(C(N)=O)cc(OCCCN(C)C(=O)CCN3C(=O)C=CC3=O)c21] | RT: 1.49 Theoretical: 931.4 Observed: 931.4 | 53% 0.94 mg 0.0008 mmol |
| 109 | 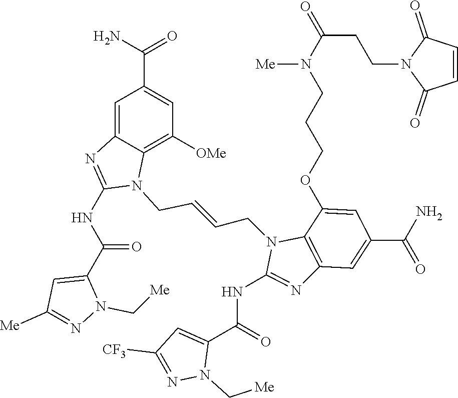 [SMILES: CCn1nc(C)cc1C(=O)Nc1nc2cc(C(N)=O)cc(OC)c2n1C/C=C/Cn1c(NC(=O)c2cc(C(F)(F)F)nn2CC)nc2cc(C(N)=O)cc(OCCCN(C)C(=O)CCN3C(=O)C=CC3=O)c21] | RT: 1.83 Theoretical: 999.4 Observed: 999.4 | 51% 1.43 mg 0.0012 mmol |
| 110 | 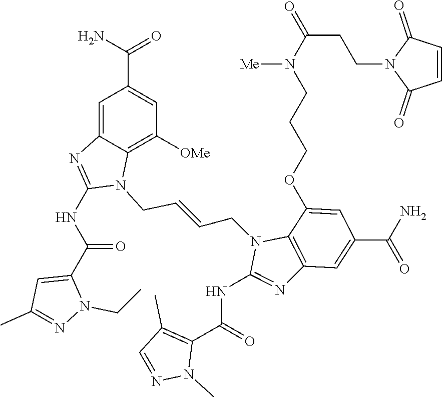 [SMILES: CCn1nc(C)cc1C(=O)Nc1nc2cc(C(N)=O)cc(OC)c2n1C/C=C/Cn1c(NC(=O)c2c(C)cnn2C)nc2cc(C(N)=O)cc(OCCCN(C)C(=O)CCN3C(=O)C=CC3=O)c21] | RT: 1.50 Theoretical: 931.4 Observed: 931.4 | 63% 3.86 mg 0.0033 mmol |
| 111 | 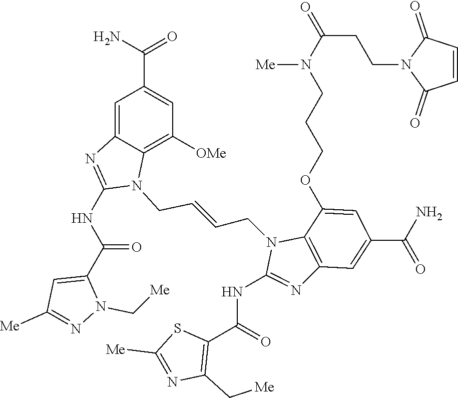 [SMILES: CCc1nc(C)sc1C(=O)Nc1nc2cc(C(N)=O)cc(OCCCN(C)C(=O)CCN3C(=O)C=CC3=O)c2n1C/C=C/Cn1c(NC(=O)c2cc(C)nn2CC)nc2cc(C(N)=O)cc(OC)c21] | RT: 1.50 Theoretical: 962.4 Observed: 962.4 | 40% 3.39 mg 0.0028 mmol |
| 112 | 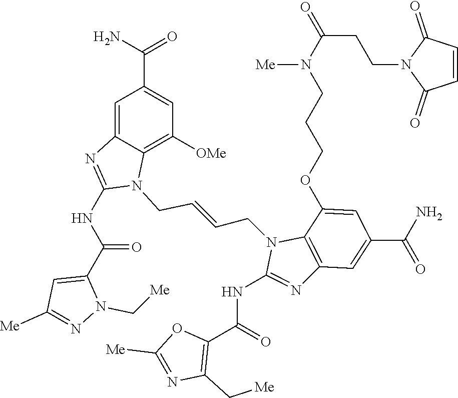 [SMILES: CCc1nc(C)oc1C(=O)Nc1nc2cc(C(N)=O)cc(OCCCN(C)C(=O)CCN3C(=O)C=CC3=O)c2n1C/C=C/Cn1c(NC(=O)c2cc(C)nn2CC)nc2cc(C(N)=O)cc(OC)c21] | RT: 1.59 Theoretical: 946.4 Observed: 946.4 | 31% 0.67 mg 0.0006 mmol |

### Synthesis of methyl (E)-1-(4-(5-carbamoyl-7-(3-(3-(2,5-dioxo-2,5-dihydro-1H-pyrrol-1-yl)-N-methylpropanamido)propoxy)-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxylate (Compound 113)

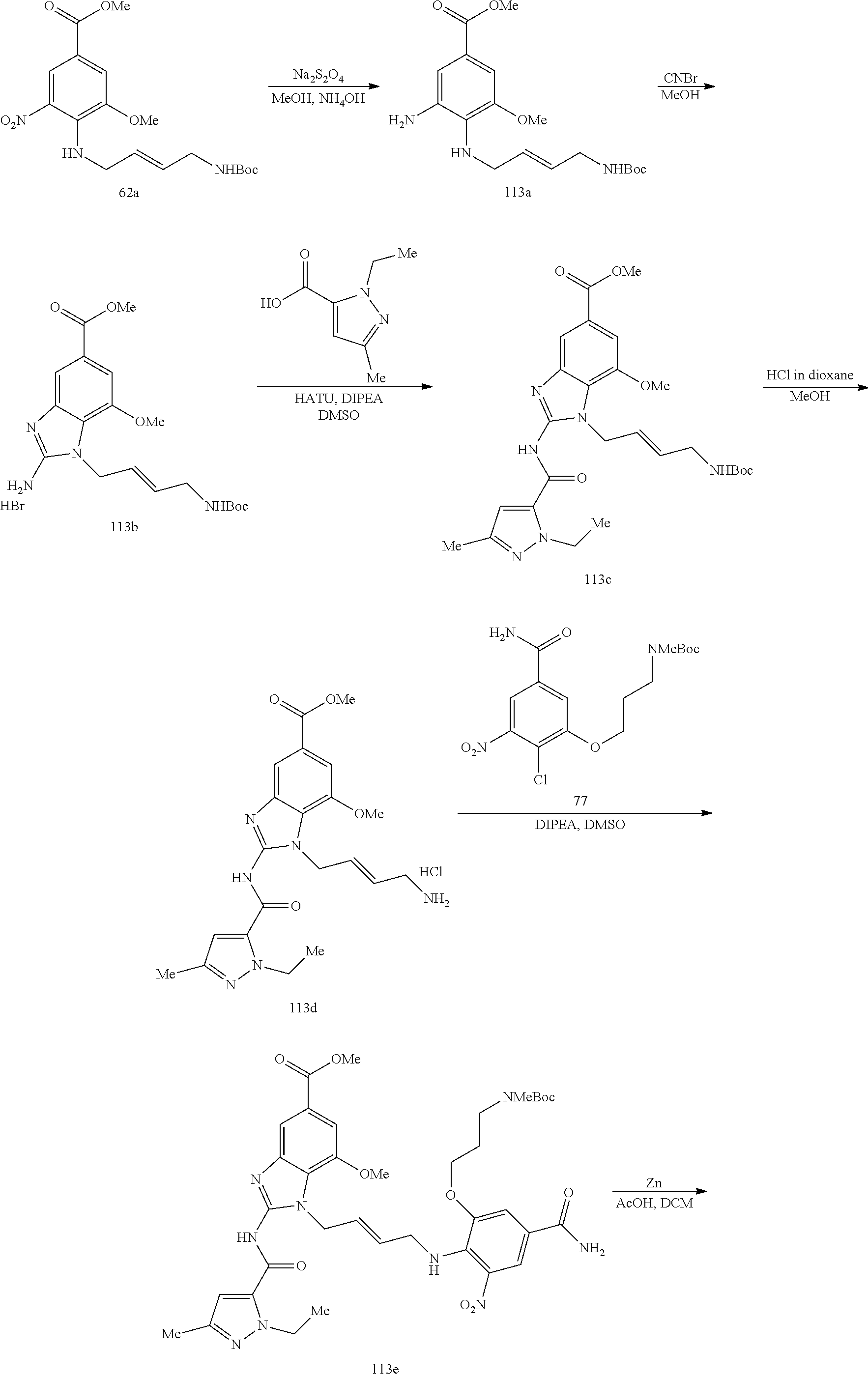

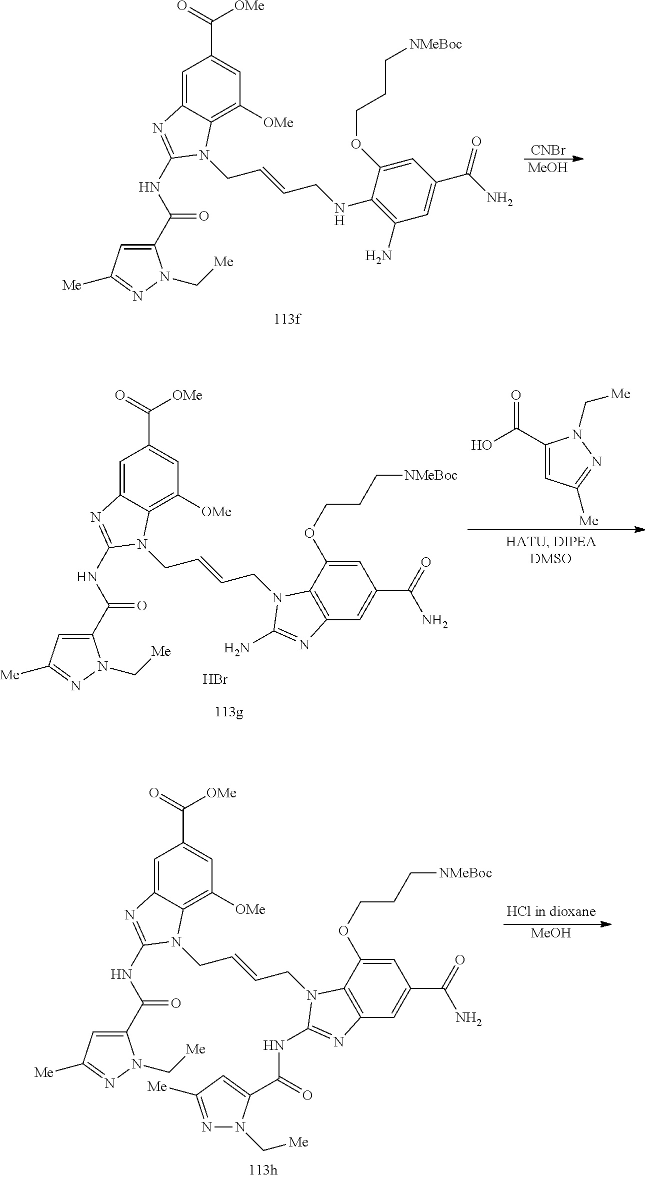

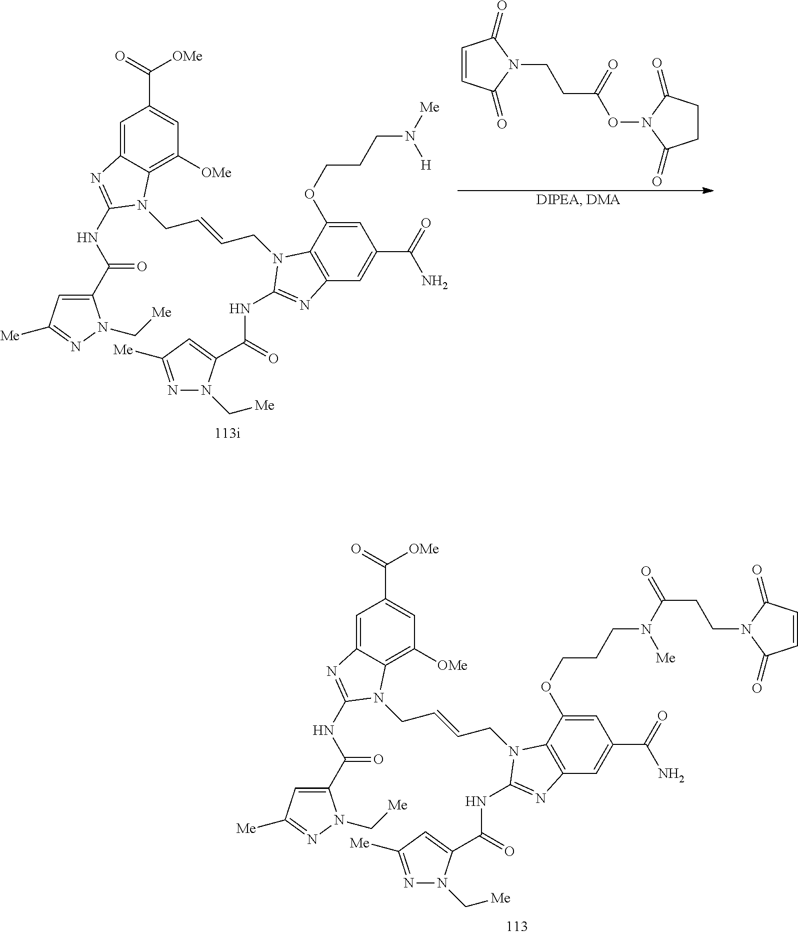

### Synthesis of methyl (E)-3-amino-4-((4-((tert-butoxycarbonyl)amino)but-2-en-1-yl)amino)-5-methoxybenzoate (Compound 113a)

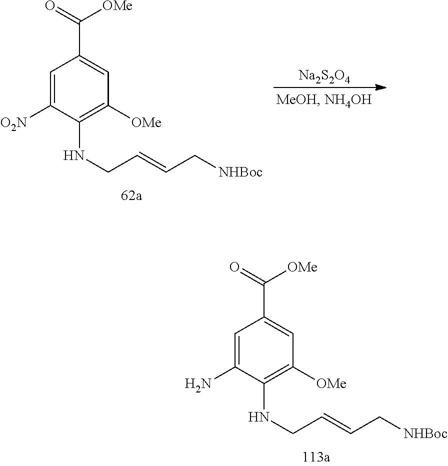 [SMILES: COC(=O)c1cc(OC)c(NC/C=C/CNC(=O)OC(C)(C)C)c([N+](=O)[O-])c1 ; "Na2S2O4" ; COC(=O)c1cc(N)c(NC/C=C/CNC(=O)OC(C)(C)C)c(OC)c1]

Compound 62a (500 mg, 1.26 mmol, 1 equiv.) was dissolved in MeOH (20 mL) and NH₄OH (6 mL). Na₂S₂O₄ (1.10 g, 6.32 mmol, 5 equiv.) in H₂O (5 mL) was slowly added and the mixture stirred at room temperature for 30 min. The reaction was monitored by UPLC-MS (Method E, ESI+). Upon completion, the mixture was filtered and concentrated. The resulting product was redissolved in EtOAc and washed with H₂O (×3). The organics were collected, dried with MgSO₄, filtered, and concentrated to afford compound 113a (343 mg, 0.938 mmol, 74% yield) as a yellow solid. The resulting product was used without further purification. UPLC-MS (Method E, ESI+): m/z [M+H]⁺=366.2 (theoretical), 366.2 (observed). HPLC retention time: 1.54 min.

### Synthesis of methyl (E)-2-amino-1-(4-((tert-butoxycarbonyl)amino)but-2-en-1-yl)-7-methoxy-1H-benzo[d]imidazole-5-carboxylate hydrobromide (Compound 113b)

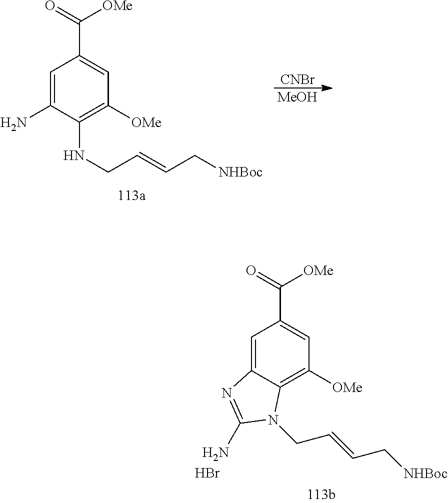 [SMILES: "MeOH" ; COC(=O)c1cc(N)c(NC/C=C/CNC(=O)OC(C)(C)C)c(OC)c1 ; "CNBr" ; Br ; COC(=O)c1cc(OC)c2c(c1)nc(N)n2C/C=C/CNC(=O)OC(C)(C)C]

Compound 113a (343 mg, 0.938 mmol, 1 equiv.) was dissolved in MeOH (9.3 mL) to which CNBr (3 M in MeCN, 0.374 mL, 1.2 equiv.) was added. The reaction stirred for 18 h at room temperature, and monitored by UPLC-MS (Method E, ESI+). Upon completion, the solution was concentrated to afford compound 113b (402 mg, 0.853 mmol, 91% yield), which was used without further purification. UPLC-MS (Method E, ESI+): m/z [M+H]⁺=391.2 (theoretical), 391.1 (observed). HPLC retention time: 1.51 min.

### Synthesis of methyl (E)-1-(4-((tert-butoxycarbonyl)amino)but-2-en-1-yl)-2-(1-ethyl-3-methyl-1H-pyrazole-5-carboxamido)-7-methoxy-1H-benzo[d]imidazole-5-carboxylate (Compound 113c)

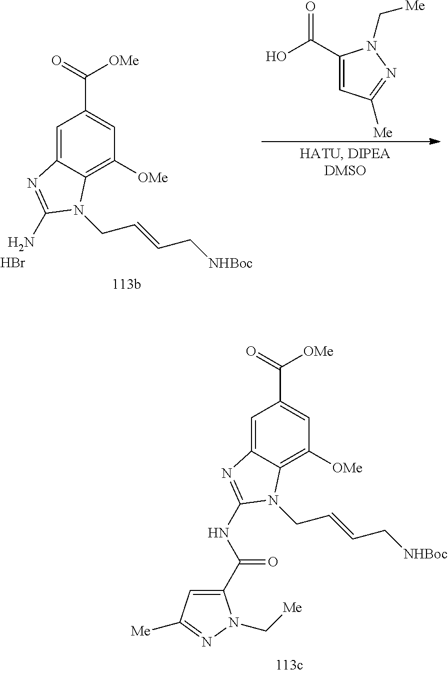 [SMILES: Br ; COC(=O)c1cc(OC)c2c(c1)nc(N)n2C/C=C/CNC(=O)OC(C)(C)C ; CCn1nc(C)cc1C(=O)O ; CCn1nc(C)cc1C(=O)Nc1nc2cc(C(=O)OC)cc(OC)c2n1C/C=C/CNC(=O)OC(C)(C)C]

Compound 113b (402 mg, 0.853 mmol, 1 equiv.), 1-ethyl-3-methyl-1H-pyrazole-5-carboxylic acid (394 mg, 2.56 mmol, 3 equiv.) and HATU (973 mg, 2.56 mmol, 3 equiv.) were dissolved in DMA (1.7 mL) in a 5 mL microwave vial. DIPEA (0.74 mL, 4.26 mmol, 5 equiv.) was added, and the reaction was heated to 80° C. in a microwave reactor for 1 h. The reaction was monitored via UPLC-MS (Method E, ESI+). Upon completion, the reaction mixture was slowly added to ice-cold water (300 mL) to precipitate compound 113c (295 mg, 0.560 mmol, 66% yield), which was used without further purification. UPLC-MS (Method E, ESI+): m/z [M+H]⁺=527.3 (theoretical), 527.1 (observed). HPLC retention time: 2.30 min.

### Synthesis of methyl (E)-1-(4-aminobut-2-en-1-yl)-2-(1-ethyl-3-methyl-1H-pyrazole-5-carboxamido)-7-methoxy-1H-benzo[d]imidazole-5-carboxylate (Compound 113d)

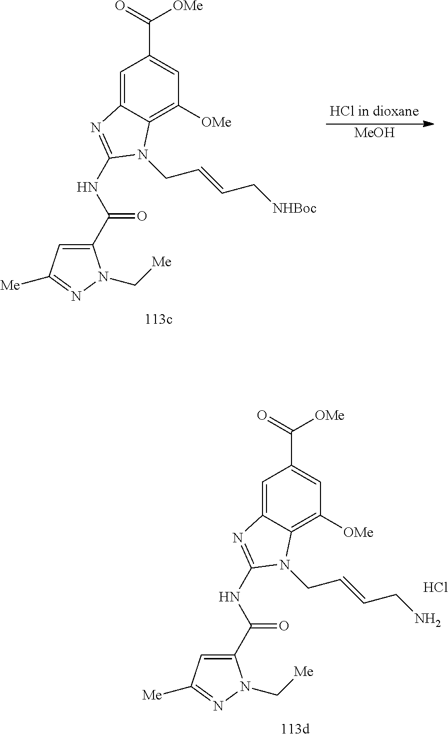 [SMILES: "MeOH" ; CCn1nc(C)cc1C(=O)Nc1nc2cc(C(=O)OC)cc(OC)c2n1C/C=C/CNC(=O)OC(C)(C)C ; CCn1nc(C)cc1C(=O)Nc1nc2cc(C(=O)OC)cc(OC)c2n1C/C=C/CN ; Cl]

Compound 113c (319 mg, 0.606 mmol, 1 equiv.) was dissolved in MeOH (1 mL), to which HCl in dioxane (4 M, 1.2 mL, 4.85 mmol, 8 equiv.) was added. The reaction was stirred at room temperature for 30 min. and was monitored by UPLC-MS (Method E, ESI+). Upon completion, the solution was concentrated and compound 113d (280 mg, 0.605 mmol, quantitative yield) was used without further purification. UPLC-MS (Method E, ESI+): m/z [M+H]⁺=427.2 (theoretical), 427.2 (observed). HPLC retention time: 1.54 min.

### Synthesis of methyl (E)-1-(4-((2-(3-((tert-butoxycarbonyl)(methyl)amino)propoxy)-4-carbamoyl-6-nitrophenyl)amino)but-2-en-1-yl)-2-(1-ethyl-3-methyl-1H-pyrazole-5-carboxamido)-7-methoxy-1H-benzo[d]imidazole-5-carboxylate (Compound 113e)

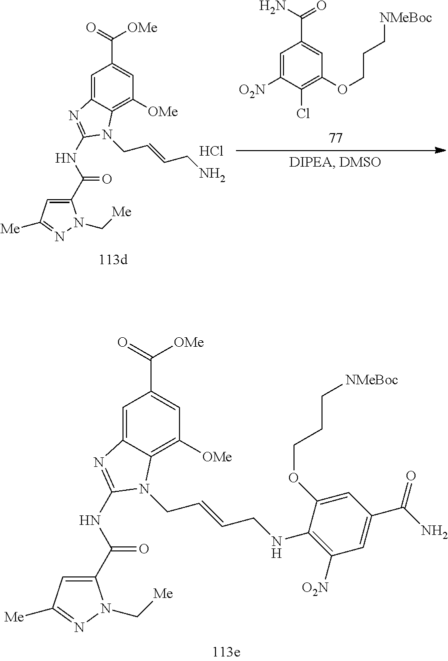

Compound 113d (280 mg, 0.605 mmol, 1 equiv.) and compound 77 (305 mg, 0.787 mmol, 1.3 equiv.) were dissolved in DMSO (3.0 mL) to which DIPEA (0.316 mL, 1.82 mmol, 3 equiv.) was added. The reaction stirred at 80° C. for 18 h and monitored via UPLC-MS (Method E, ESI+). Upon completion, AcOH (0.30 mL) was added, and the product was purified by prepHPLC (Method I) using 0.05% TFA as the additive. Pure fractions were collected, frozen, and lyophilized to afford compound 113e (58.6 mg, 0.0753 mmol, 12% yield) as an orange solid. UPLC-MS (Method E, ESI+): m/z [M+H]⁺=778.3 (theoretical), 778.4 (observed). HPLC retention time: 1.88 min.

### Synthesis of methyl (E)-1-(4-((2-amino-6-(3-((tert-butoxycarbonyl)(methyl)amino)propoxy)-4-carbamoylphenyl)amino)but-2-en-1-yl)-2-(1-ethyl-3-methyl-1H-pyrazole-5-carboxamido)-7-methoxy-1H-benzo[d]imidazole-5-carboxylate (Compound 113f)

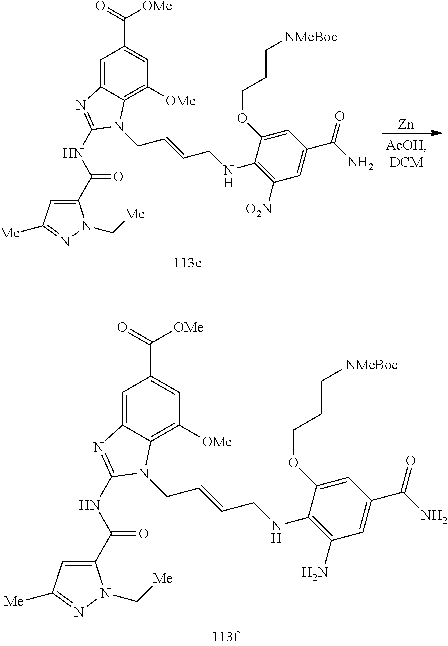

Compound 113e (58.6 mg, 0.0753 mmol, 1 equiv.) was dissolved in a 1:1 mixture of AcOH/DCM (0.75 mL) and cooled to 0° C. Zn (49.2 mg, 0.753 mmol, 10 equiv.) was added and the mixture was allowed to warm to room temperature while stirring for 30 min. The reaction was monitored via UPLC-MS (Method E, ESI+). Upon completion, the solution was concentrated and redissolved in DCM to be purified by flash chromatography (25 g SiO₂ column, 0-40% MeOH:NH₄OH (10:1) in DCM) to afford compound 113f (28.3 mg, 0.378 mmol, 50% yield). UPLC-MS (Method E, ESI+): m/z [M+H]⁺=748.4 (theoretical), 748.4 (observed). HPLC retention time: 1.84 min.

### Synthesis of methyl (E)-1-(4-(2-amino-7-(3-((tert-butoxycarbonyl)(methyl)amino)propoxy)-5-carbamoyl-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxylate (Compound 113g)

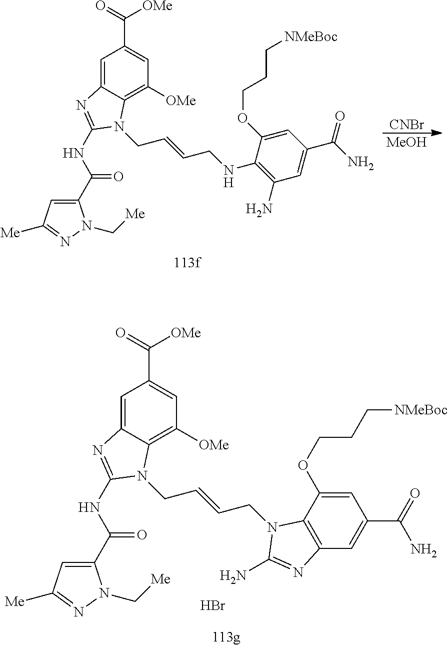

Compound 113f (28.3 mg, 0.378 mmol, 1 equiv.) was dissolved in MeOH (0.38 mL) to which CNBr (3 M in MeCN, 15 μL, 0.0454 mmol, 1.2 equiv.) was added. The reaction stirred at room temperature for 18 h and was monitored via UPLC-MS (Method E, ESI+). Upon completion, the solution was concentrated to afford product 113g (30.7 mg, 0.360 mmol, quantitative yield), which was used without further purification. UPLC-MS (Method E, ESI+): m/z [M+H]⁺=773.4 (theoretical), 773.4 (observed). HPLC retention time: 1.53 min.

### Synthesis of methyl (E)-1-(4-(7-(3-((tert-butoxycarbonyl)(methyl)amino)propoxy)-5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxylate (Compound H)

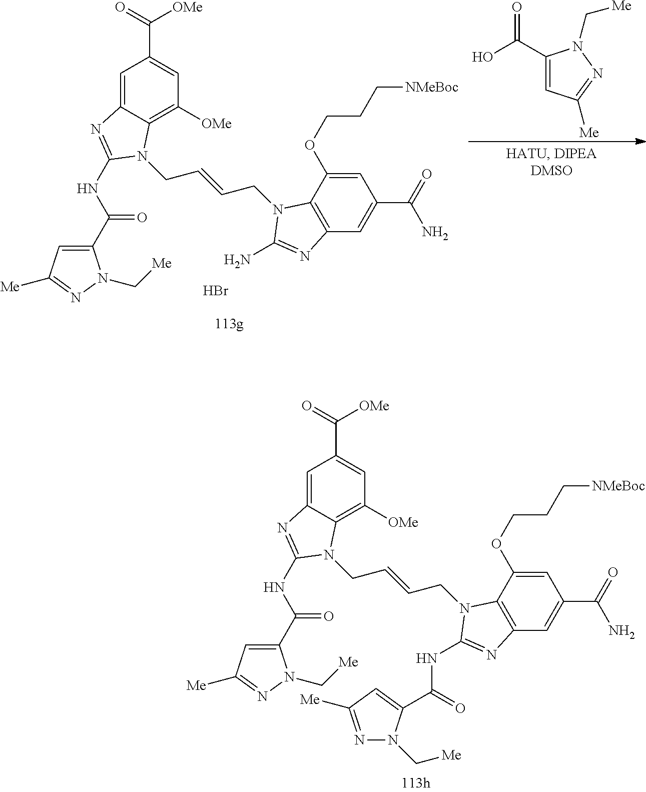

Compound 113g (30.7 mg, 0.0360 mmol, 1 equiv.), 1-ethyl-3-methyl-1H-pyrazole-5-carboxylic acid (22.1 mg, 0.144 mmol, 4 equiv.), and HATU (54.6 mg, 0.144 mmol, 4 equiv.) were dissolved in DMA (0.50 mL) in a 2 mL microwave vial. DIPEA (0.025 mL, 0.144 mmol, 4 equiv.) was added, and the reaction was heated in a microwave reactor at 80° C. for 1 h. The reaction was monitored via UPLC-MS (Method E, ESI+). Upon completion, the product was purified by prepHPLC (Method H) using 0.05% TFA as the additive. Pure fractions were collected, frozen, and lyophilized to afford compound 113h (6.52 mg, 0.0064 mmol, 18% yield) as a white solid. UPLC-MS (Method E, ESI+): m/z [M+H]⁺=909.4 (theoretical), 909.5 (observed). HPLC retention time: 1.90 min.

### Synthesis of methyl (E)-1-(4-(5-carbamoyl-2-(1-ethyl-3-methyl-1H-pyrazole-5-carboxamido)-7-(3-(methylamino)propoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxylate (Compound 113i)

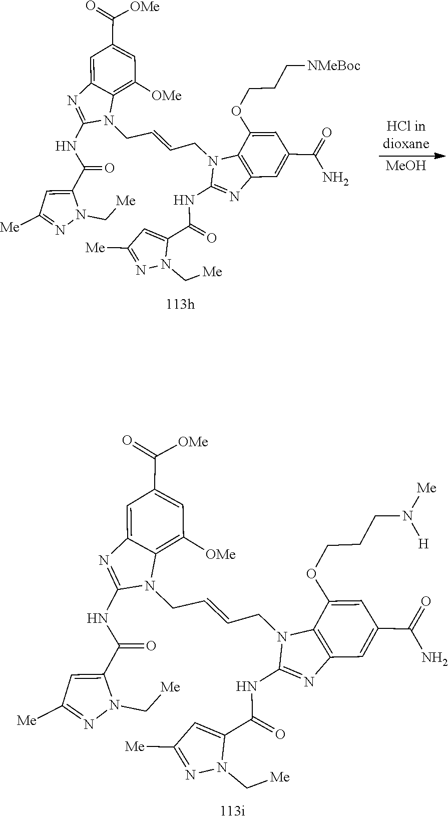

Compound 113h (3.02 mg, 0.0030 mmol, 1 equiv.) was dissolved in MeOH (0.30 mL) to which HCl in dioxane (4 M, 6.00 μL, 0.0236 mmol, 8 equiv.) was added. The reaction stirred for 30 min at room temperature and monitored via UPLC-MS (Method E, ESI+). Upon completion, the product was purified via prepHPLC (Method G) using 0.05% TFA as the additive. Pure fractions were collected, frozen, and lyophilized to afford compound 113i (1.35 mg, 0.0013 mmol, 44% yield) as a white solid. UPLC-MS (Method E, ESI+): m/z [M+H]⁺=809.4 (theoretical), 809.4 (observed). HPLC retention time: 1.57 min.

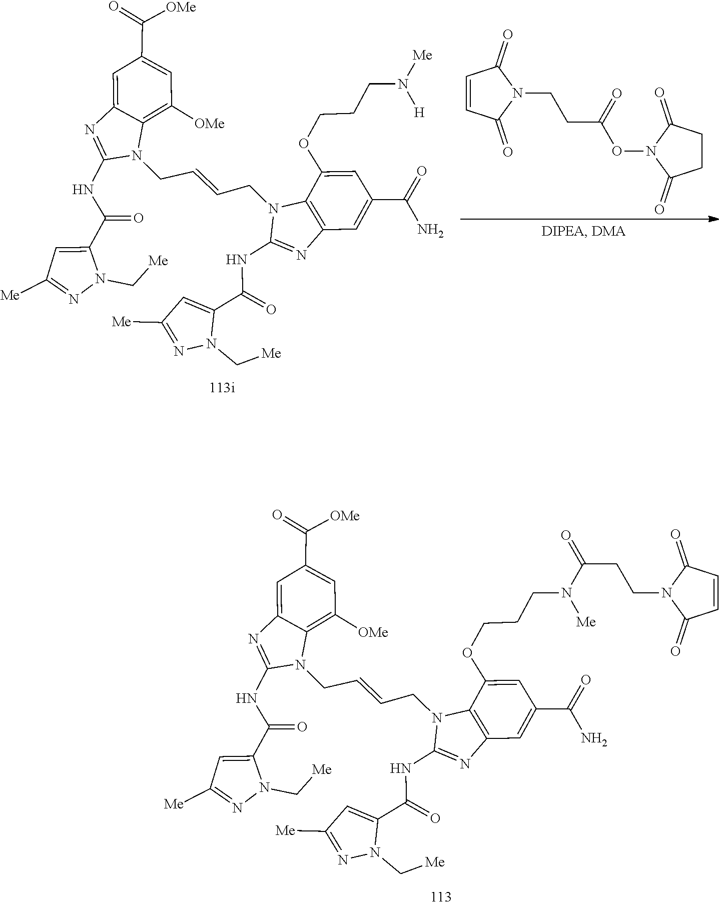

Compound 113i (7.53 mg, 0.0085 mmol, 1 equiv.) and MP-OSu (4.55 mg, 0.0171 mmol, 2 equiv.) were dissolved in DMA (0.854 mL), and DIPEA (42.7 μL, 0.0074 mmol, 5 equiv.) was added. The reaction stirred at room temperature for 18 h and monitored by UPLC-MS (Method E, ESI+). Upon completion, AcOH (42 μL) was added, and the product was purified via prepHPLC (Method G) using 0.05% TFA as the additive. Pure fractions were collected, frozen, and lyophilized to afford compound 113 (4.43 mg, 0.0041 mmol, 48% yield) as a white solid. UPLC-MS (Method E, ESI+): m/z [M+H]⁺=960.4 (theoretical), 960.5 (observed). HPLC retention time: 1.79 min.

### Linker Library Synthesis (Compounds 114-124).

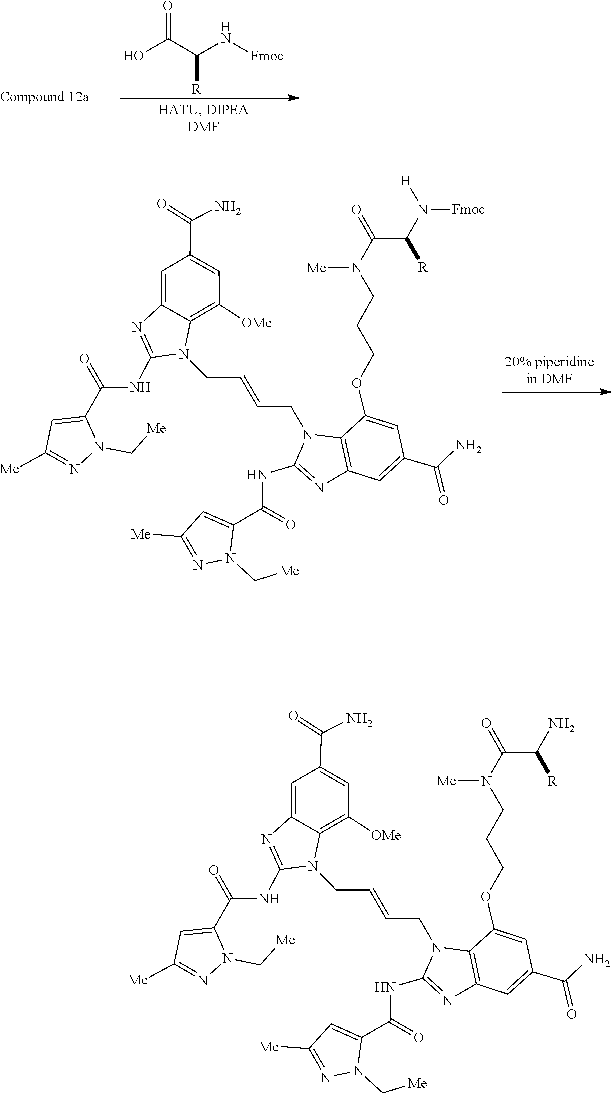

Amide coupling (General Method 10): A mixture of Compound 12a (1 equiv.), HATU (2 equiv.), DIPEA (5 equiv.), the appropriate L-amino acid (2 equiv.) was prepared in DMF (0.02 M in 12a) and stirred at room temperature overnight. The solvent was removed in vacuo, and resulting product used in the next step without further purification.

Fmoc deprotection (General Method 11): The resulting Fmoc-protected amine was dissolved in 20% piperidine in DMF (1 mL) and stirred at room temperature for 15 minutes. The solvent was removed in vacuo and the product purified via prep HPLC (Method H, 5-95% in MeCN in H₂O in 0.05% TFA).

| | UPLC-MS | |
|---|---|---|
| Compound | [M + H]⁺ | Yield |
| 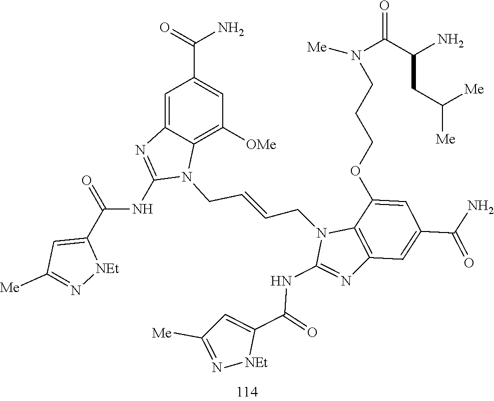 | RT: 1.83 min Theoretical: 907.5 Observed: 907.5 | 18.6 mg (58%) |
| 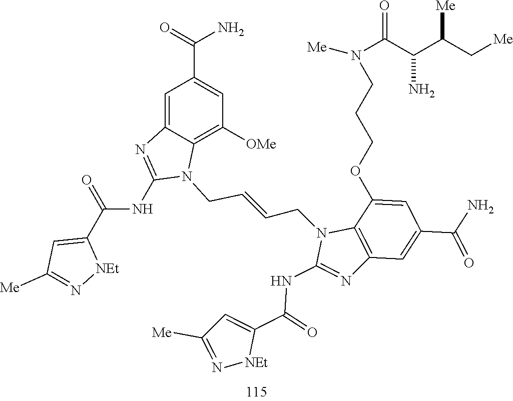 | RT: 1.77 Theoretical: 907.5 Observed: 907.5 | 24.9 mg (78%) |
| 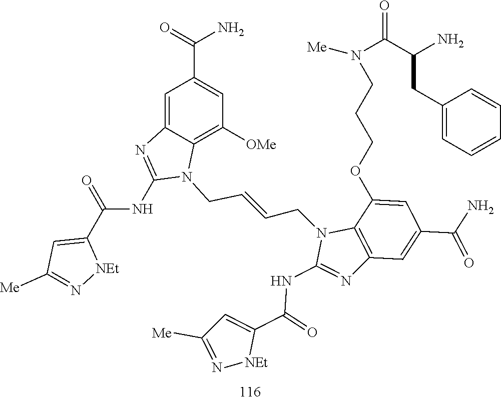 | RT: 1.76 Theoretical: 941.5 Observed: 941.5 | 13.5 mg (28%) |
| 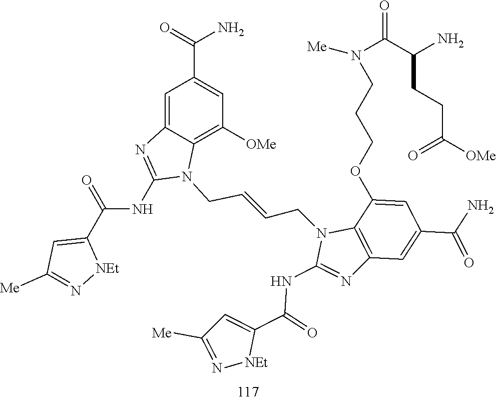 | RT: 2.05 Theoretical: 937.4 Observed: 937.5 | 9.8 mg (35%) |
| 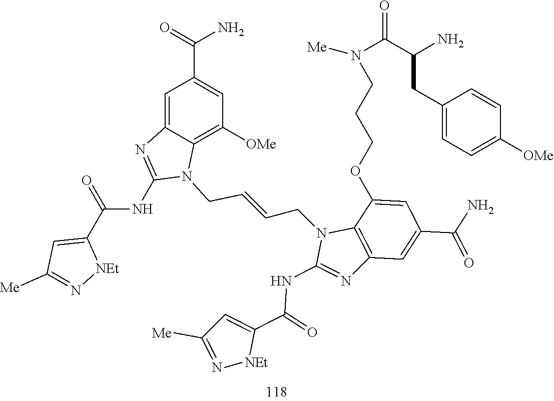 | RT: 1.93 Theoretical: 971.5 Observed: 971.5 | 18.6 mg (64%) |
| 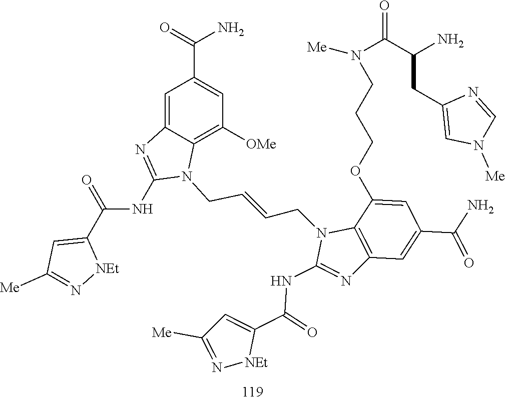 | RT: 1.93 Theoretical: 945.5 Observed: 945.5 | 19.4 (64%) |
| 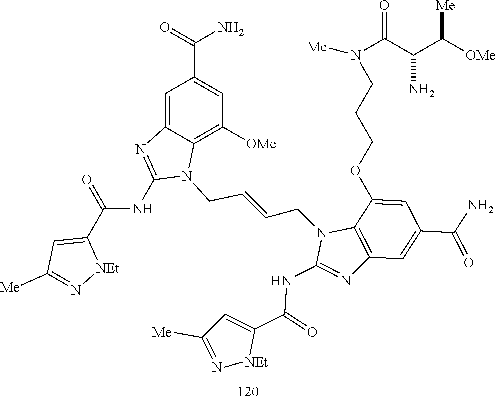 | RT: 2.05 Theoretical: 909.4 Observed: 909.5 | 24.5 mg (76.4%) |

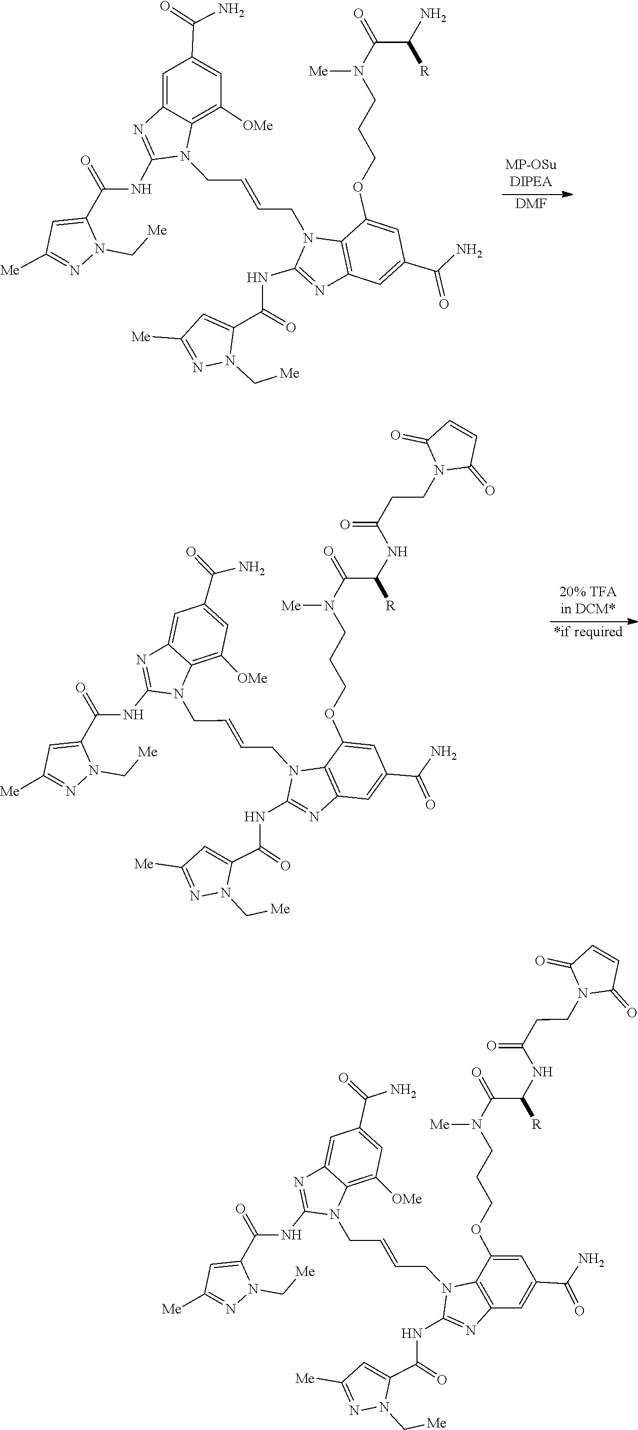

Synthesis of maleimide containing drug-linkers (compounds 121-125) was performed according to General Method 9.

| | UPLC-MS | |
|---|---|---|
| Compound | [M + H]⁺ | Yield |
| 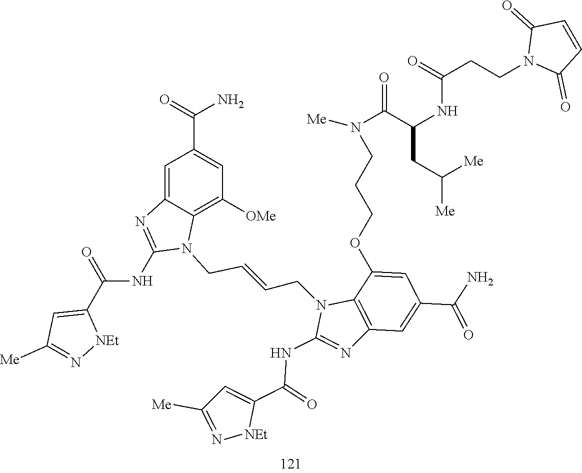 | RT: 2.16 Theoretical: 1058.5 Observed: 1058.5 | 1.5 mg (42%) |
| 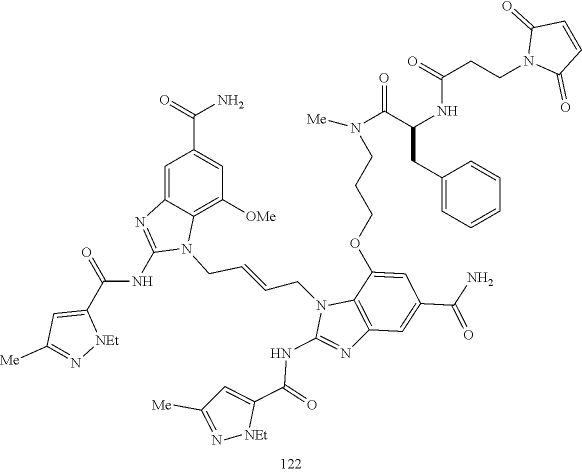 | RT: 2.00 Theoretical: 1092.5 Observed: 1092.5 | 0.5 mg (21%) |
| 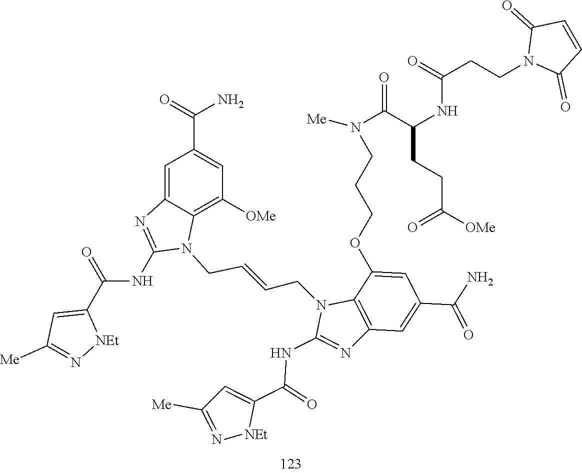 | RT: 2.21 Theoretical: 1088.5 Observed: 1088.5 | 1.1 mg (32%) |
| 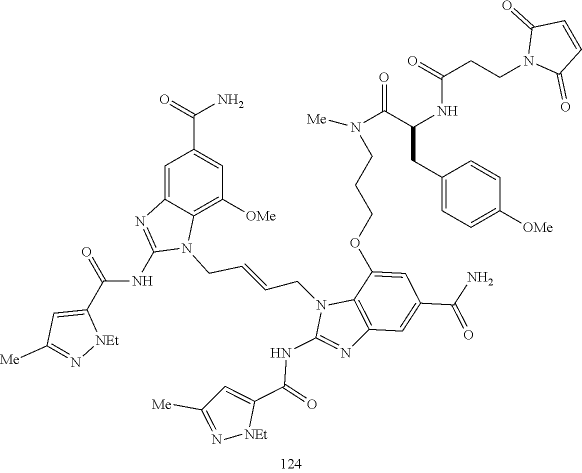 | RT: 1.81 Theoretical: 1122.5 Observed: 1122.6 | 1.1 mg (32%) |
| 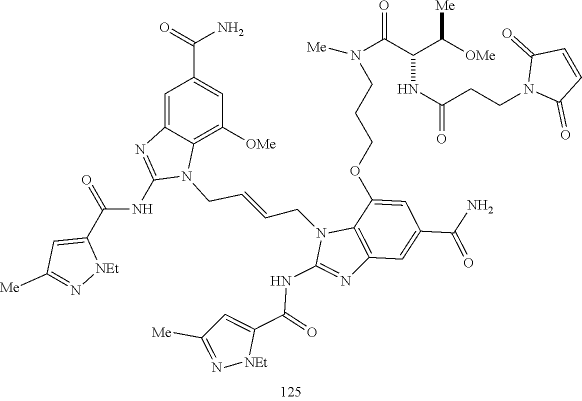 | RT: 1.79 Theoretical: 1060.5 Observed: 1060.5 | 0.3 mg (8.6%) |

### (E)-1-(4-(5-carbamoyl-7-(3-(3-(2,5-dioxo-2,5-dihydro-1H-pyrrol-1-yl)-N-methylpropanamido)propoxy)-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxylic acid (Compound 126)

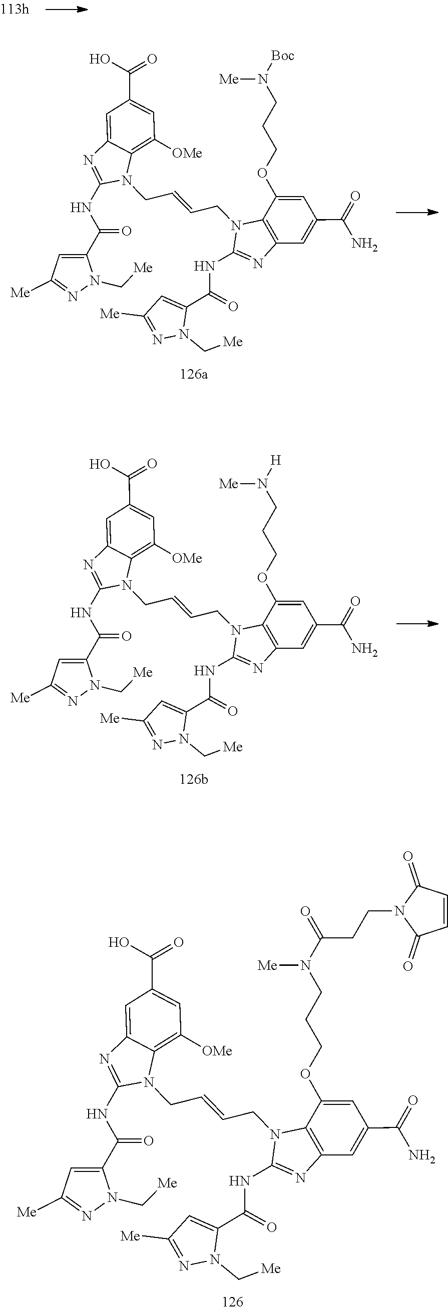 [SMILES: CCn1nc(C)cc1C(=O)Nc1nc2cc(C(=O)O)cc(OC)c2n1C/C=C/Cn1c(NC(=O)c2cc(C)nn2CC)nc2cc(C(N)=O)cc(OCCCN(C)C(=O)OC(C)(C)C)c21 ; CCn1nc(C)cc1C(=O)Nc1nc2cc(C(=O)O)cc(OC)c2n1C/C=C/Cn1c(NC(=O)c2cc(C)nn2CC)nc2cc(C(N)=O)cc(OCCCNC)c21 ; CCn1nc(C)cc1C(=O)Nc1nc2cc(C(=O)O)cc(OC)c2n1C/C=C/Cn1c(NC(=O)c2cc(C)nn2CC)nc2cc(C(N)=O)cc(OCCCN(C)C(=O)CCN3C(=O)C=CC3=O)c21]

### Synthesis of compound 126a

Compound 113h (25.44 mg, 0.0249 mmol, 1 equiv.) was dissolved in MeOH (166 μL). An aqueous solution of 1M LiOH (200 μL, 8 equiv.) was added and the reaction was stirred at 80° C. for 2h. Upon completion, the solution was concentrated under reduced pressure and purified by prepHPLC (Method H) using 0.05% TFA as the additive. Pure fractions were collected, frozen, and lyophilized to afford compound 126a (7.1 mg, 0.0071 mmol, 28% yield) as a white solid. UPLC-MS (Method E, ESI+): m/z [M+H]⁺=895.4 (theoretical), 895.6 (observed). HPLC retention time: 1.97 min.

### Synthesis of compound 126b

Compound 126b was prepared following the same procedure used to prepare compound 113i. UPLC-MS (Method E, ESI+): m/z [M+H]⁺=795.4 (theoretical), 795.6 (observed). HPLC retention time: 1.40 min.

### Synthesis of compound 126

Compound 126 was prepared following the same procedure used to prepare compound 113. UPLC-MS (Method E, ESI+): m/z [M+H]⁺=946.4 (theoretical), 946.6 (observed). HPLC retention time: 1.68 min.

### Synthesis of tert-butyl (E)-(3-((5-carbamoyl-2-(1-ethyl-3-methyl-1H-pyrazole-5-carboxamido)-1-(4-(2-(1-ethyl-3-methyl-1H-pyrazole-5-carboxamido)-5-(N-methylsulfamoyl)-1H-benzo[d]imidazol-1-yl)but-2-en-1-yl)-1H-benzo[d]imidazol-7-yl)oxy)propyl)(methyl)carbamate (Compound 127)

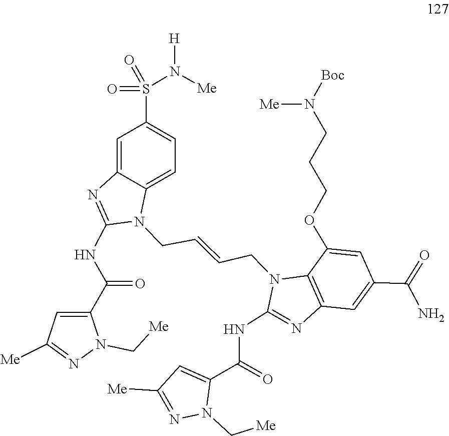 [SMILES: CCn1nc(C)cc1C(=O)Nc1nc2cc(S(=O)(=O)NC)ccc2n1C/C=C/Cn1c(NC(=O)c2cc(C)nn2CC)nc2cc(C(N)=O)cc(OCCCN(C)C(=O)OC(C)(C)C)c21]

Compound 127 was prepared following the same procedures as compound 25f substituting 4-chloro-N-methyl-3-nitrobenzenesulfonamide for 4-chloro-3-nitrobenzenesulfonamide. UPLC-MS (Method E, ESI+): m/z [M+H]⁺=914.4 (theoretical), 914.6 (observed). HPLC retention time: 1.80 min.

### Synthesis of (E)-7-(3-(3-(2,5-dioxo-2,5-dihydro-1H-pyrrol-1-yl)-N-methylpropanamido)propoxy)-2-(1-ethyl-3-methyl-1H-pyrazole-5-carboxamido)-1-(4-(2-(1-ethyl-3-methyl-1H-pyrazole-5-carboxamido)-5-(N-methylsulfamoyl)-1H-benzo[d]imidazol-1-yl)but-2-en-1-yl)-1H-benzo[d]imidazole-5-carboxamide (Compound 128)

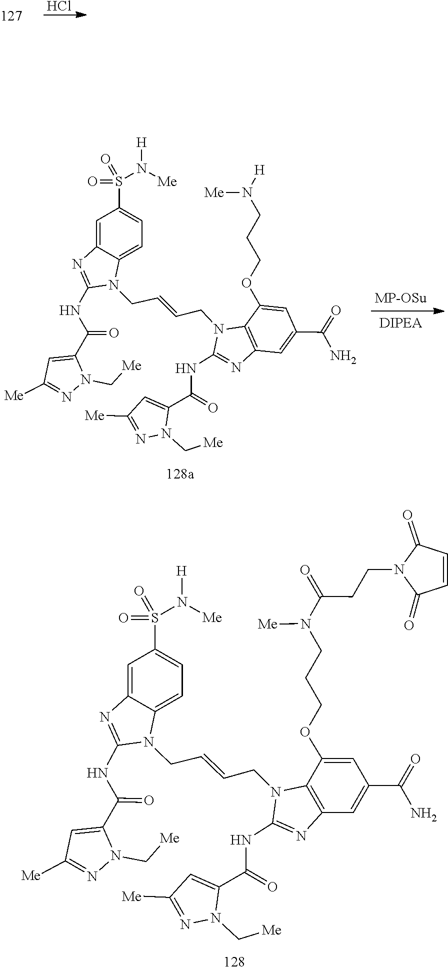 [SMILES: Cl ; CCn1nc(C)cc1C(=O)Nc1nc2cc(S(=O)(=O)NC)ccc2n1C/C=C/Cn1c(NC(=O)c2cc(C)nn2CC)nc2cc(C(N)=O)cc(OCCCNC)c21 ; CCn1nc(C)cc1C(=O)Nc1nc2cc(S(=O)(=O)NC)ccc2n1C/C=C/Cn1c(NC(=O)c2cc(C)nn2CC)nc2cc(C(N)=O)cc(OCCCN(C)C(=O)CCN3C(=O)C=CC3=O)c21]

### Synthesis of 128a

Compound 128a was prepared following the same procedure used to prepare compound 66b. UPLC-MS (Method E, ESI+): m/z [M+H]⁺=814.4 (theoretical), 814.5 (observed). HPLC retention time: 1.53 min.

### Synthesis of 128

Compound 128 was prepared following the same procedure used to prepare compound 12. UPLC-MS (Method E, ESI+): m/z [M+H]⁺=965.4 (theoretical), 965.6 (observed). HPLC retention time: 1.60 min.

### Synthesis of S-(1-(3-((3-((5-carbamoyl-2-(1-ethyl-3-methyl-1H-pyrazole-5-carboxamido)-1-((E)-4-(2-(1-ethyl-3-methyl-1H-pyrazole-5-carboxamido)-7-methoxy-5-(methoxycarbonyl)-1H-benzo[d]imidazol-1-yl)but-2-en-1-yl)-1H-benzo[d]imidazol-7-yl)oxy)propyl)(methyl)amino)-3-oxopropyl)-2,5-dioxopyrrolidin-3-yl)-L-cysteine (Compound 129)

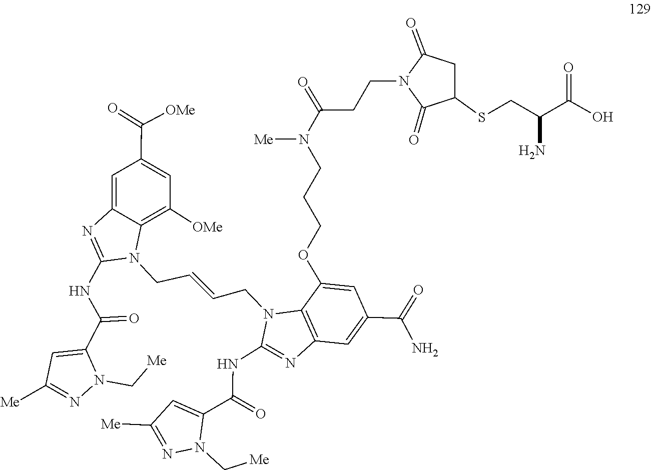 [SMILES: CCn1nc(C)cc1C(=O)Nc1nc2cc(C(=O)OC)cc(OC)c2n1C/C=C/Cn1c(NC(=O)c2cc(C)nn2CC)nc2cc(C(N)=O)cc(OCCCN(C)C(=O)CCN3C(=O)CC(SC[C@H](N)C(=O)O)C3=O)c21]

Compound 129 was prepared following General Method 6. UPLC-MS (Method E, ESI+): m/z [M+H]⁺=1081.4 (theoretical), 1081.6 (observed). HPLC retention time: 1.88 min.

### Synthesis of 1-((E)-4-(7-(3-(3-(3-(((R)-2-amino-2-carboxyethyl)thio)-2,5-dioxopyrrolidin-1-yl)-N-methylpropanamido)propoxy)-5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxylicacid (Compound 130)

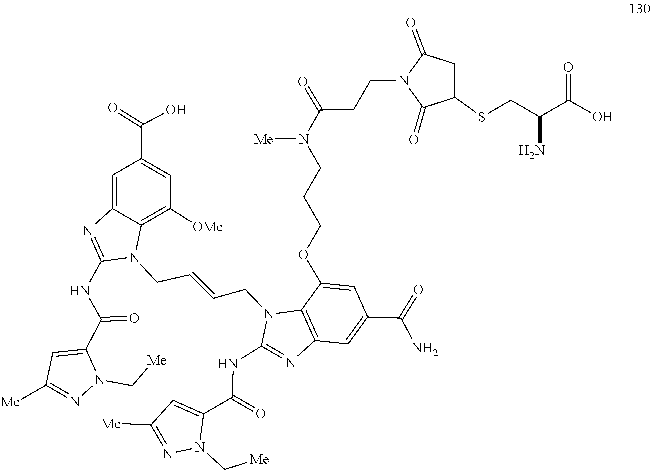 [SMILES: CCn1nc(C)cc1C(=O)Nc1nc2cc(C(=O)O)cc(OC)c2n1C/C=C/Cn1c(NC(=O)c2cc(C)nn2CC)nc2cc(C(N)=O)cc(OCCCN(C)C(=O)CCN3C(=O)CC(SC[C@H](N)C(=O)O)C3=O)c21]

Compound 130 was prepared following General Method 6. UPLC-MS (Method E, ESI+): m/z [M+H]⁺=1067.4 (theoretical), 1067.6 (observed). HPLC retention time: 1.49 min.

### Synthesis of (E)-1-(4-(5-carbamoyl-2-(1-ethyl-3-methyl-1H-pyrazole-5-carboxamido)-1H-benzo[d]imidazol-1-yl)but-2-en-1-yl)-7-(3-(6-(2,5-dioxo-2,5-dihydro-1H-pyrrol-1-yl)-N-methylhexanamido)propoxy)-2-(1-ethyl-3-methyl-1H-pyrazole-5-carboxamido)-1H-benzo[d]imidazole-5-carboxamide (Compound 131)

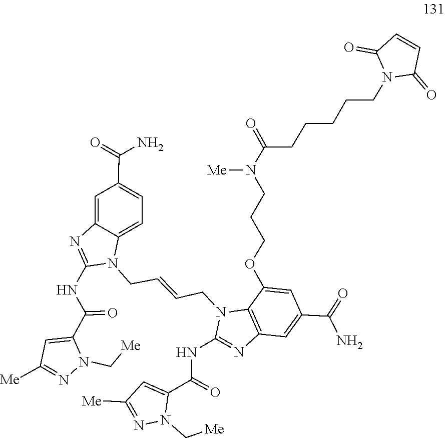 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN(C)C(=O)CCCCCN3C(=O)C=CC3=O)c21]

Compound 131 was prepared following the same procedure as compound 12 substituting 2,5-dioxopyrrolidin-1-yl 6-(2,5-dioxo-2,5-dihydro-1H-pyrrol-1-yl)hexanoate for 2,5-dioxopyrrolidin-1-yl 3-(2,5-dioxo-2,5-dihydro-1H-pyrrol-1-yl)propanoate. UPLC-MS (Method E, ESI+): m/z [M+H]⁺=987.5 (theoretical), 987.7 (observed). HPLC retention time: 1.85 min.

### Synthesis of S-(1-(6-((3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)(methyl)amino)-6-oxohexyl)-2,5-dioxopyrrolidin-3-yl)-L-cysteine (Compound 132)

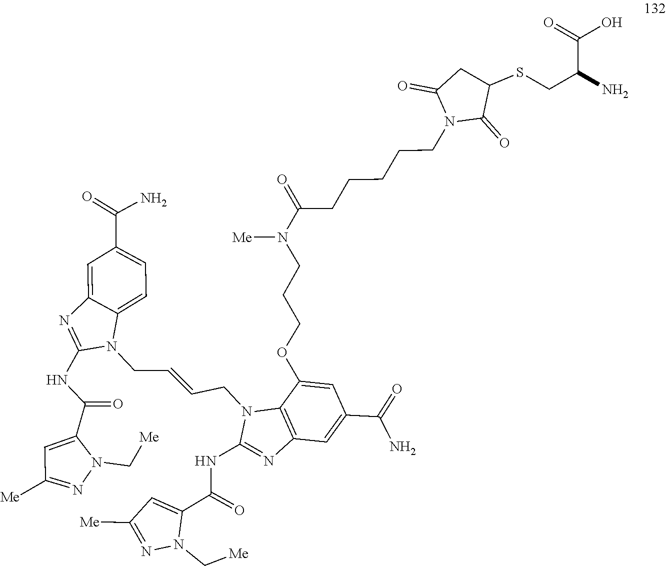 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN(C)C(=O)CCCCCN3C(=O)CC(SC[C@H](N)C(=O)O)C3=O)c21]

Compound 132 was prepared following General Method 6. UPLC-MS (Method E, ESI+): m/z [M+H]⁺=1108.5 (theoretical), 1108.7 (observed). HPLC retention time: 1.42 min.

### Synthesis of (E)-1-(4-(5-carbamoyl-2-(1-ethyl-3-methyl-1H-pyrazole-5-carboxamido)-7-methoxy-1H-benzo[d]imidazol-1-yl)but-2-en-1-yl)-7-(3-(N-cyclopropyl-3-(2,5-dioxo-2,5-dihydro-1H-pyrrol-1-yl)propanamido)propoxy)-2-(1-ethyl-3-methyl-1H-pyrazole-5-carboxamido)-1H-benzo[d]imidazole-5-carboxamide (Compound 133)

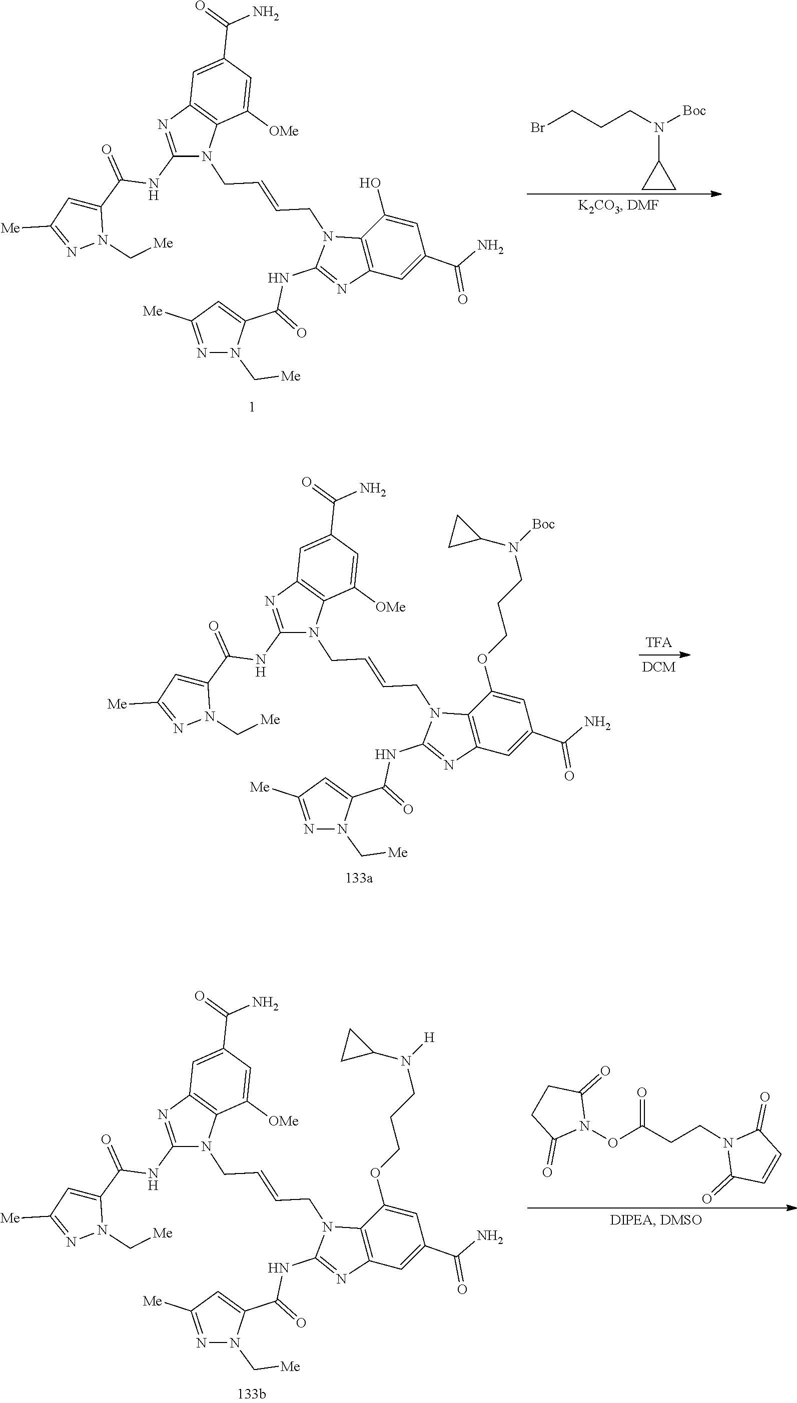 [SMILES: CCn1nc(C)cc1C(=O)Nc1nc2cc(C(N)=O)cc(O)c2n1C/C=C/Cn1c(NC(=O)c2cc(C)nn2CC)nc2cc(C(N)=O)cc(OC)c21 ; CC(C)(C)OC(=O)N(CCCBr)C1CC1 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C(=O)OC(C)(C)C)C3CC3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCNC3CC3)c21 ; O=C(CCN1C(=O)C=CC1=O)ON1C(=O)CCC1=O]

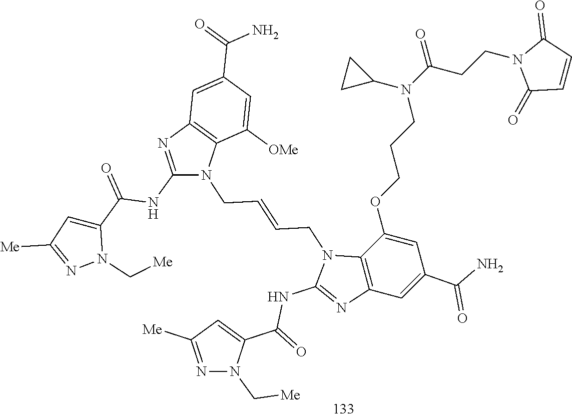 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C(=O)CCN3C(=O)C=CC3=O)C3CC3)c21]

### Synthesis of 133a

An oven-dried 4 mL vial was charged with 1 (10 mg, 0.0105 mmol, 1 equiv), potassium carbonate (7.3 mg, 0.0526 mmol, 5 equiv.), and tert-butyl N-(3-bromopropyl)-N-cyclopropyl-carbamate (0.49 mL of a 9 mg/mL solution in DMF, 0.0158 mmol, 1.50 equiv.) and starting materials were dissolved in DMF (0.50 mL). The reaction was stirred overnight at 55° C. and purified by preparatory HPLC (Method B), after which it was frozen and lyophilized to afford compound 133a (8.8 mg, 0.0077 mmol, 73% yield). UPLC-MS (Method D, ESI+): m/z [M+H]⁺=920.45 (theoretical), 920.64 (observed). HPLC retention time: 2.32 min. Synthesis of 133b

An oven-dried 4 mL vial was charged with 133a (8.8 mg, 0.0077 mmol) and 20% TFA in DCM (100 μL). The reaction was stirred for 30 minutes at room temperature and purified by preparatory HPLC (Method B), after which it was frozen and lyophilized to afford compound 133b (5.0 mg, 0.0043 mmol, 56% yield). UPLC-MS (Method D, ESI+): m/z [M+H]⁺=820.40 (theoretical), 820.49 (observed). HPLC retention time: 1.29 min.

### Synthesis of 133

An oven-dried 8 mL vial was charged with 133b (3.3 mg, 0.0085 mmols, 1 equiv.) which was dissolved in DMSO (1 mL) and a solution of 2,5-dioxopyrrolidin-1-yl 3-(2,5-dioxo-2,5-dihydro-1H-pyrrol-1-yl)propanoate in DMSO (10 mM in DMSO, 0.43 mL, 0.0043 mmol, 1.5 equiv.) and DIPEA (1.5 μL, 0.00851 mmol, 3 equiv.). The reaction was heated to 30° C. overnight, quenched with acetic acid and purified by preparatory HPLC (Method B), after which the compound was frozen and lyophilized to afford 133 (1.9 mg, 0.00158 mmol, 56% yield).

UPLC-MS (Method D, ESI+): m/z [M+H]⁺=971.43 (theoretical), 971.48 (observed). HPLC retention time: 1.99 min.

### Synthesis of 3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)-N-(4-(((2S,3R,4S,5S,6S)-6-carboxy-3,4,5-trihydroxytetrahydro-2H-pyran-2-yl)oxy)-2-(3-(3-(2,5-dioxo-2,5-dihydro-1H-pyrrol-1-yl)propanamido)propanamido)benzyl)-N,N-dimethylpropan-1-aminium 2,2,2-trifluoroacetate (Compound 134)

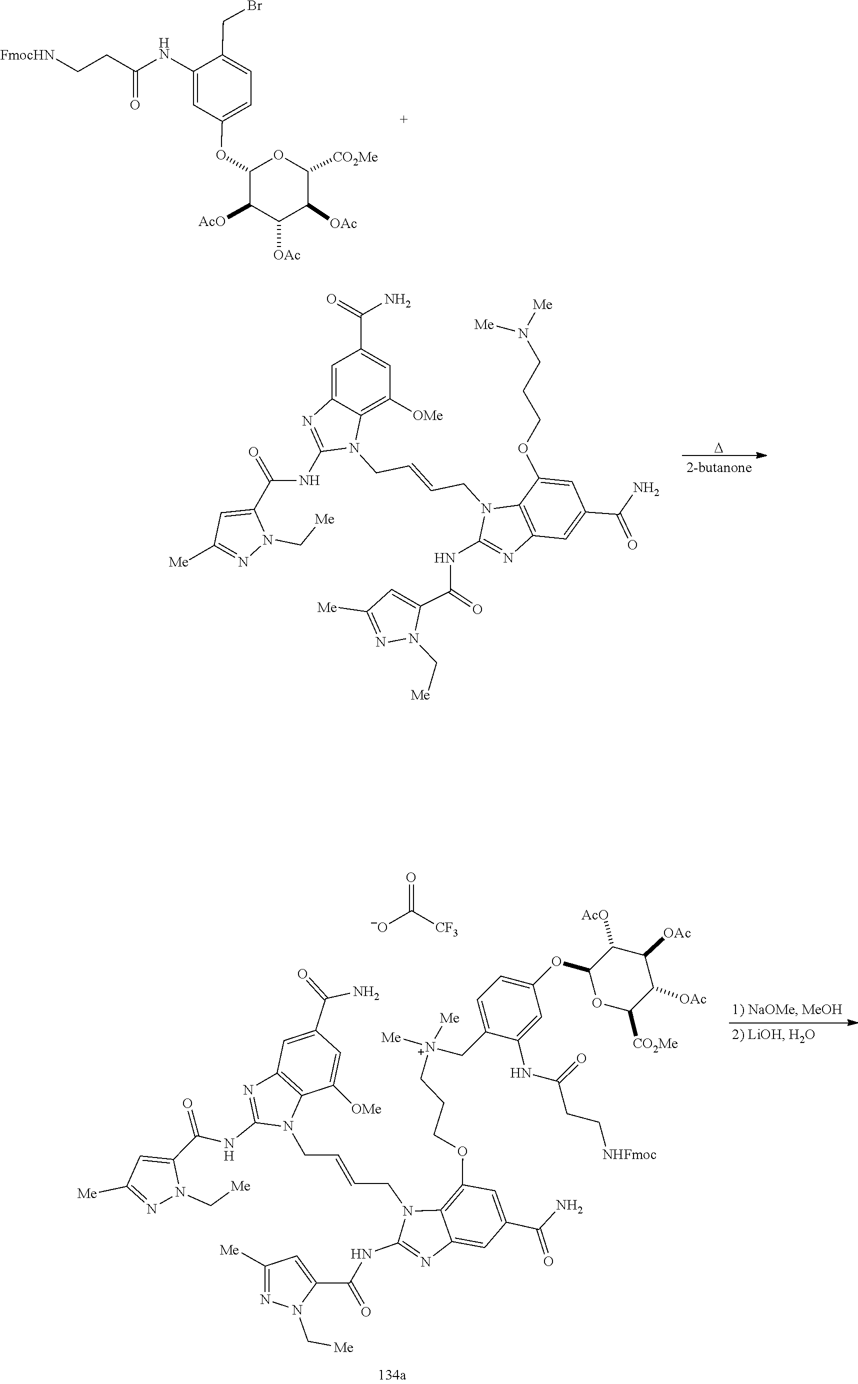

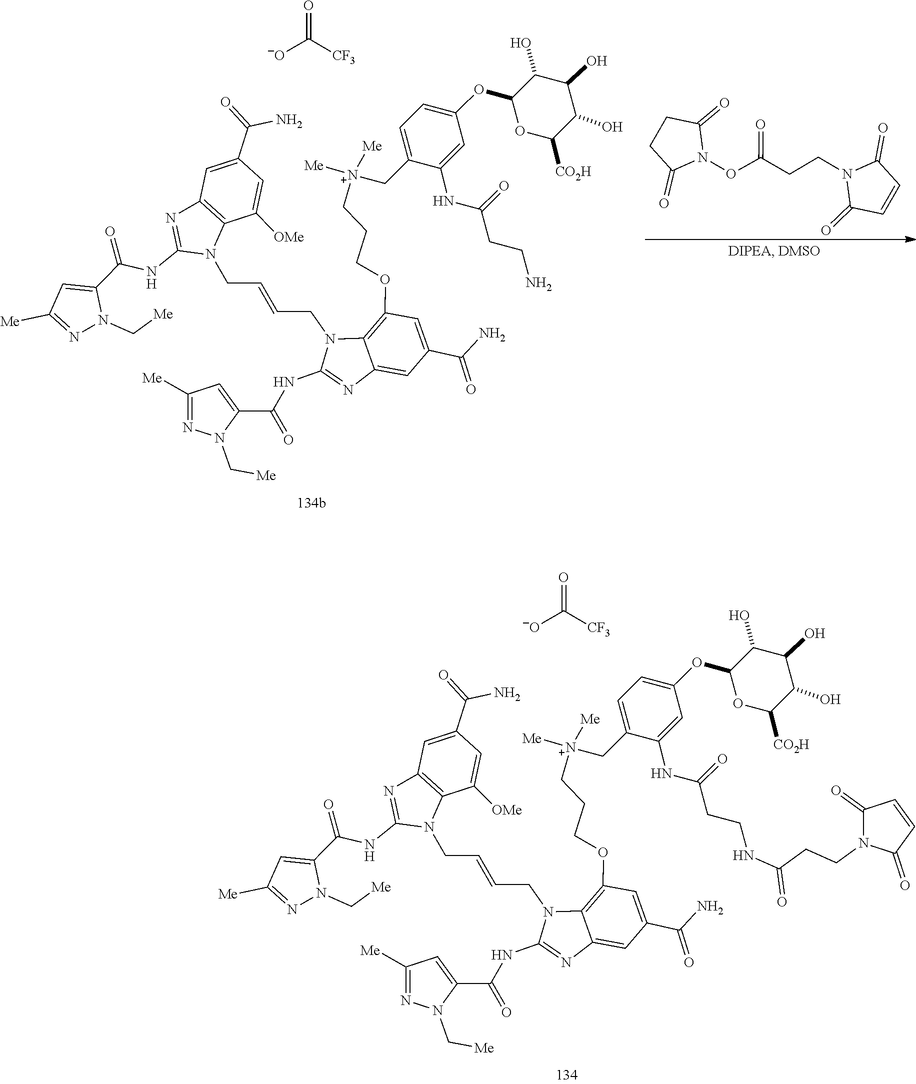 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCC[N+](C)(C)Cc3ccc(O[C@@H]4O[C@H](C(=O)O)[C@@H](O)[C@H](O)[C@H]4O)cc3NC(=O)CCN)c21 ; O=C([O-])C(F)(F)F ; O=C(CCN1C(=O)C=CC1=O)ON1C(=O)CCC1=O ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCC[N+](C)(C)Cc3ccc(O[C@@H]4O[C@H](C(=O)O)[C@@H](O)[C@H](O)[C@H]4O)cc3NC(=O)CCNC(=O)CCN3C(=O)C=CC3=O)c21 ; O=C([O-])C(F)(F)F]

### Synthesis of 134a

An oven-dried 8 mL vial was charged with (E)-1-(4-(5-carbamoyl-2-(1-ethyl-3-methyl-1H-pyrazole-5-carboxamido)-7-methoxy-1H-benzo[d]imidazol-1-yl)but-2-en-1-yl)-7-(3-(dimethylamino)propoxy)-2-(1-ethyl-3-methyl-1H-pyrazole-5-carboxamido)-1H-benzo[d]imidazole-5-carboxamide (20 mg, 0.0248 mmol, 1 equiv., prepared as previously described WO2017/175147, example 39, page 291) and (2S,3R,4S,5S,6S)-2-(3-(3-((((9H-fluoren-9-yl)methoxy)carbonyl)amino)propanamido)-4-(bromomethyl)phenoxy)-6-(methoxycarbonyl)tetrahydro-2H-pyran-3,4,5-triyl triacetate (60.3 mg, 0.0743 mmol, 3 equiv, prepared as previously described, Mol Cancer Ther 2016 15(5), 938-945) and azeotroped with anhydrous acetonitrile. To the vial was added 2-butanone (2.5 mL) and the solution was heated to 100° C. overnight. The compound was directly purified by preparatory HPLC (Method B), frozen and lyophilized to afford 134a (11.3 mg, 0.0070 mmol, 28% yield). UPLC-MS (Method E, ESI+): m/z [M+H]⁺=1538.64 (theoretical), 1538.83 (observed). HPLC retention time: 2.55 min

### Synthesis of 134b

An oven-dried 4 mL vial was charged with 134a (4.5 mg, 0.0094 mmol, 1 equiv.) and dissolved in anhydrous MeOH (0.5 mL). The vial was cooled in an acetonitrile/dry-ice bath at −40° C. and 0.5 M NaOMe (19 μL, 0.0094 mmol, 1 equiv) was added. The reaction was stirred for 1 hour before it was warmed to room temperature and LiOH (1 M in H₂O, 31 μL, 0.031 mmols, 3 equiv.) was added. The reaction was stirred at room temperature for 1 hour and then directly purified by preparatory HPLC (Method B) then frozen and lyophilized to afford 134b (5.8 mg, 0.0049 mmol, 48% yield). UPLC-MS (Method E, ESI+): m/z [M+H]⁺=1176.52 (theoretical), 1176.76 (observed). HPLC retention time: 1.29 min

### Synthesis of 134

134b (5.8 mg, 0.0038 mmols, 1 equiv.) was added to an oven-dried 4 mL vial and dissolved in DMSO (1 mL) and then MP-OSu (10 mM in DMSO, 0.57 mL, 0.0057 mL, 1.5 equiv.) and DIPEA (2 μL, 0.0115 mmol, 3 equiv.) were added. The solution was stirred for 30 min, quenched with acetic acid and purified by preparatory HPLC (Method B), then frozen and lyophilized to afford 134 (3.6 mg, 0.0023 mmol, 61% yield). UPLC-MS (Method E, ESI+): m/z [M+H]⁺=1327.55 (theoretical), 1327.77 (observed). HPLC retention time: 1.38 min.

### Synthesis of (E)-7-(2-(azetidin-3-yl)ethoxy)-2-(1-ethyl-3-methyl-1H-pyrazole-5-carboxamido)-1-(4-(2-(1-ethyl-3-methyl-1H-pyrazole-5-carboxamido)-5-sulfamoyl-1H-benzo[d]imidazol-1-yl)but-2-en-1-yl)-1H-benzo[d]imidazole-5-carboxamide (Compound 135)

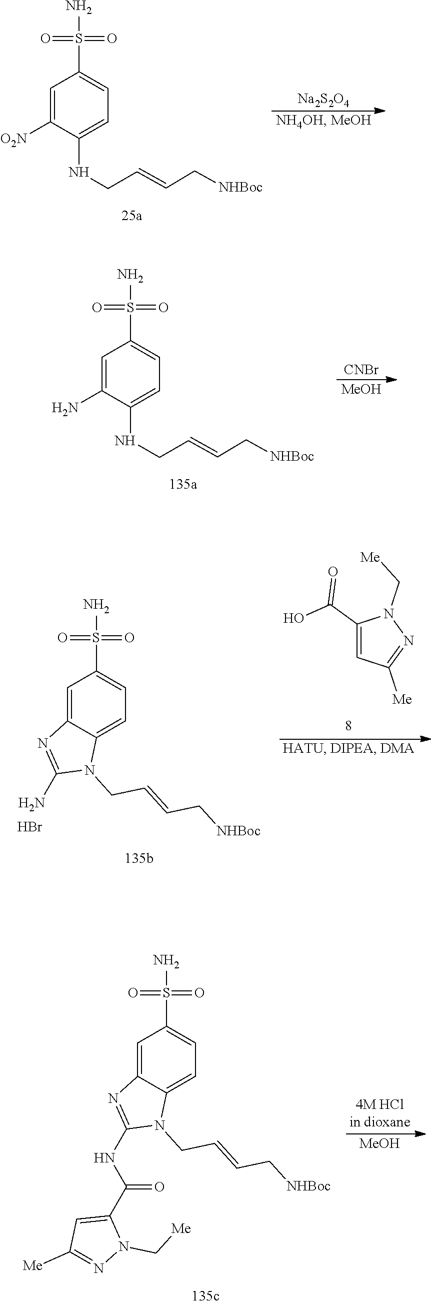

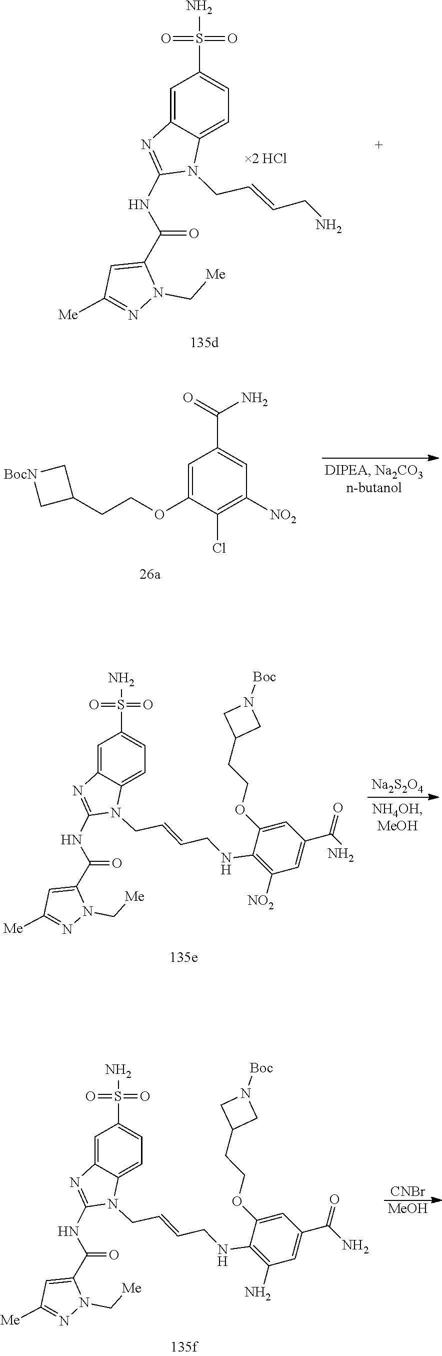

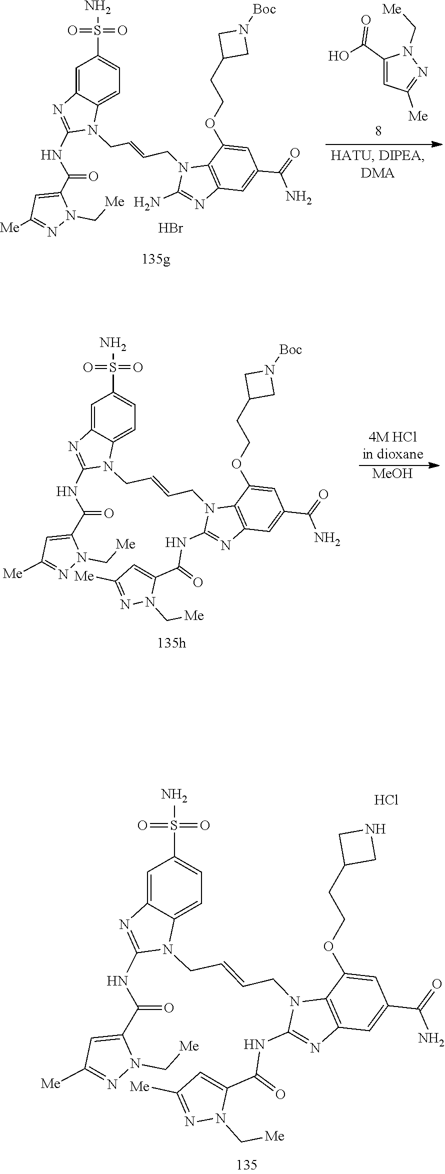

### Synthesis of 135a

To a solution of 25a (1.61 g, 4.18 mmol, 1 equiv.) in MeOH (63 mL) and aq. NH₄OH (21 mL) was added aq. Na₂S₂O₄ (1 M, 21 mL, 21 mmol, 5 equiv.). The mixture was stirred for 1 hour at 30° C., and the reaction was monitored by UPLC-MS (Method E, ESI+). Upon completion, the solution was filtered over celite and washed with MeOH. The filtrate was concentrated and the product was purified by flash chromatography (dry loaded on celite, Sfar HC Duo SiO₂ column, 10:1 MeOH:NH₄OH gradient in DCM) to yield 135a (774 mg, 2.17 mmol, 52% yield). LC-MS (Method E, ESI+): m/z [M+H]⁺=357.2 (theoretical), 357.3 (observed). HPLC retention time: 1.44 min.

### Synthesis of 135b

To a solution of compound 135a (774 mg, 2.17 mmol, 1 equiv.) in MeOH (4 mL) was added cyanogen bromide in MeCN (3 M, 1.5 mL, 4.35 mmol, 2 equiv.). The solution stirred for 18 hours at 30° C. and was monitored via UPLC-MS (Method E, ESI+). Upon completion, solvent was removed in vacuo to yield 135b (1.0 g, 2.25 mmol, quantitative yield). LC-MS (Method E, ESI+): m/z [M+H]⁺=382.2 (theoretical), 382.2 (observed). HPLC retention time: 1.12 min.

### Synthesis of 135c

A microwave vial was charged with a solution of 135b (1.0 g, 2.25 mmol, 1 equiv.) in DMA (11 mL), to which was added compound 8 (1.0 g, 6.74 mmol, 3 equiv.), HATU (2.6 g, 6.74 mmol, 3 equiv.) and DIPEA (1.2 mL, 6.74 mmol, 3 equiv.). This mixture was heated to 80° C. for 1 hour in a microwave reactor. Upon completion, 135c was isolated by precipitation with cold water (1.0 g, 1.93 mmol, 86% yield). LC-MS (Method E, ESI+): m/z [M+H]⁺=518.2 (theoretical), 518.3 (observed). HPLC retention time: 1.60 min.

### Synthesis of 135d

To a solution of 135c (1.0 g, 1.93 mmol, 1 equiv.) in MeOH (3.3 mL) was added HCl in dioxane (4 M, 5.3 mL, 21 mmol, 8 equiv.). The mixture stirred for 1 hour at 30° C. Upon completion, solvent was removed in vacuo and 135d (1.2 g, 2.65 mmol, quantitative yield) was used without further purification. LC-MS (Method E, ESI+): m/z [M+H]⁺=418.2 (theoretical), 418.2 (observed). HPLC retention time: 1.09 min.

### Synthesis of 135e

Compounds 135d (200 mg, 0.408 mmol, 1 equiv.) and 26a (245 mg, 0.612 mmol, 1.5 equiv.) were dissolved in n-butanol (2.0 mL) in a 5 mL microwave vial to which Na₂CO₃ (130 mg, 1.22 mmol, 3 equiv.) and DIPEA (0.36 mL, 2.04 mmol, 5 equiv.) were added. The reaction was heated via microwave reactor at 140° C. for 3 hours. The resulting product was filtered and washed with MeOH and DCM. The filtrate was concentrated and purified via flash chromatography (dry loaded on celite, Sfar HC Duo SiO₂ column, 10:1 MeOH:NH₄OH gradient in DCM) to yield 135e (51 mg, 0.0651 mmol, 16% yield). LC-MS (Method E, ESI+): m/z [M+H]⁺=781.3 (theoretical), 781.4 (observed). HPLC retention time: 1.72 min.

### Synthesis of 135f

Compound 135f (30 mg, 0.0402 mmol, 62% yield) was prepared using the same procedure as 135a, using 135e (51 mg, 0.0651 mmol, 1 equiv.) as the starting material. LC-MS (Method E, ESI+): m/z [M+H]⁺=751.3 (theoretical), 751.4 (observed). HPLC retention time: 1.46 min.

### Synthesis of 135g

Compound 135g (34 mg, 00394 mmol, quantitative yield) was prepared using the same procedure as 135b, using 135f (30 mg, 0.0402 mmol, 1 equiv.) as the starting material. LC-MS (Method E, ESI+): m/z [M+H]⁺=776.3 (theoretical), 776.4 (observed). HPLC retention time: 1.54 min.

### Synthesis of 135h

Compound 135h was prepared using the same procedure as 135c, using 135g (17 mg, 0.0197 mmol, 1 equiv.) as the starting material. Upon completion, the product was purified by preparatory HPLC (Method H). Pure fractions were collected, frozen and lyophilized to afford 135h (2.34 mg, 0.0021 mmol, 10% yield) as a white powder. LC-MS (Method E, ESI+): m/z [M+H]⁺=912.4 (theoretical), 912.5 (observed). HPLC retention time: 1.65 min.

### Synthesis of 135

Compound 135h (2.34 mg, 0.0021 mmol, 1 equiv.) was dissolved in MeOH (0.21 mL) and HCl in dioxane (4 M, 4.1 μL, 0.0164 mmol, 8 equiv.) was added. The solution was heated to 40° C. for 1 hour. Then solvent was removed in vacuo and 135 (1.86 mg, 0.0020 mmol, quantitative yield) was used without further purification. LC-MS (Method E, ESI+): m/z [M+H]⁺=812.3 (theoretical), 812.4 (observed). HPLC retention time: 1.26 min.

### Synthesis of (E)-7-(2-(azetidin-3-yl)ethoxy)-2-(1,3-dimethyl-1H-pyrazole-5-carboxamido)-1-(4-(2-(1-ethyl-3-methyl-1H-pyrazole-5-carboxamido)-5-sulfamoyl-1H-benzo[d]imidazol-1-yl)but-2-en-1-yl)-1H-benzo[d]imidazole-5-carboxamide (Compound 136)

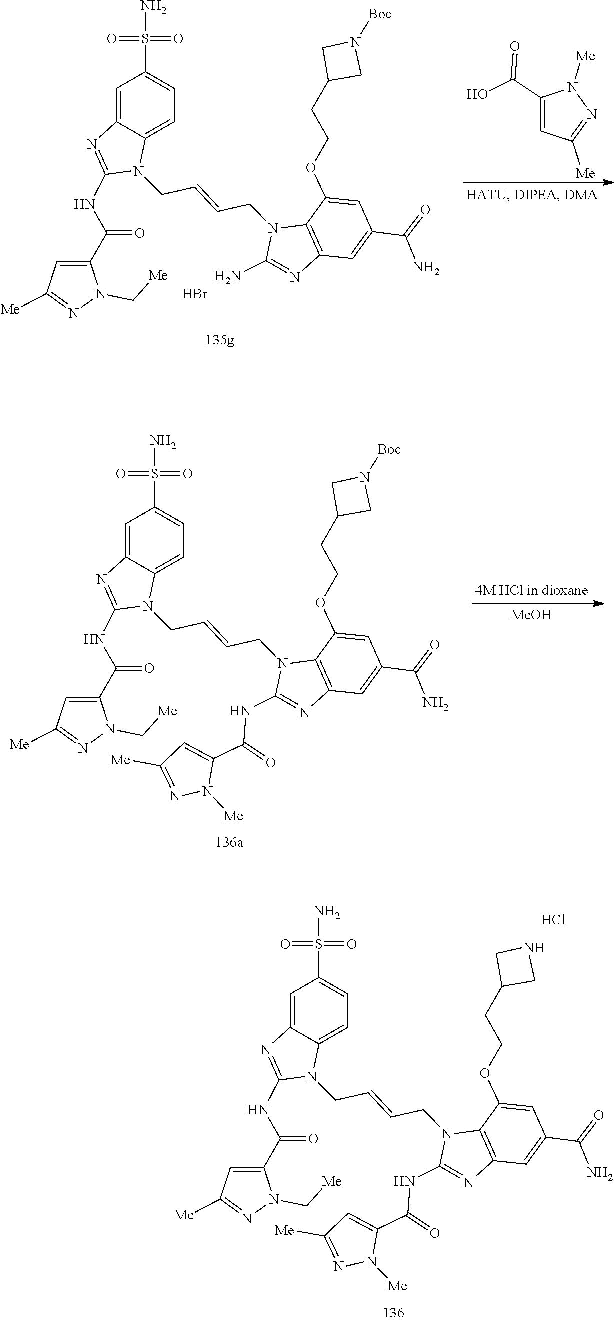

### Synthesis of 136a

Compound 136a (3.15 mg, 0.0028 mmol, 14% yield) was prepared using the same procedure as 135h, using 135g (17 mg, 0.0197 mmol, 1 equiv.) and 1, 3-dimethyl-1H-pyrazole-5-carboxyllic acid as the starting materials. LC-MS (Method E, ESI+): m/z [M+H]⁺=898.4 (theoretical), 898.5 (observed). HPLC retention time: 1.62 min.

### Synthesis of 136

Compound 136 (2.09 mg, 0.0023 mmol, 82% yield) was prepared using the same procedure as 135, using 136a (3.15 mg, 0.0028 mmol, 1 equiv.) as the starting material. LC-MS (Method E, ESI+): m/z [M+H]⁺=798.3 (theoretical), 798.4 (observed). HPLC retention time: 1.24 min.

### Synthesis of (E)-7-(3-(azetidin-3-yloxy)propoxy)-2-(1-ethyl-3-methyl-1H-pyrazole-5-carboxamido)-1-(4-(2-(1-ethyl-3-methyl-1H-pyrazole-5-carboxamido)-5-sulfamoyl-1H-benzo[d]imidazol-1-yl)but-2-en-1-yl)-1H-benzo[d]imidazole-5-carboxamide (Compound 137)

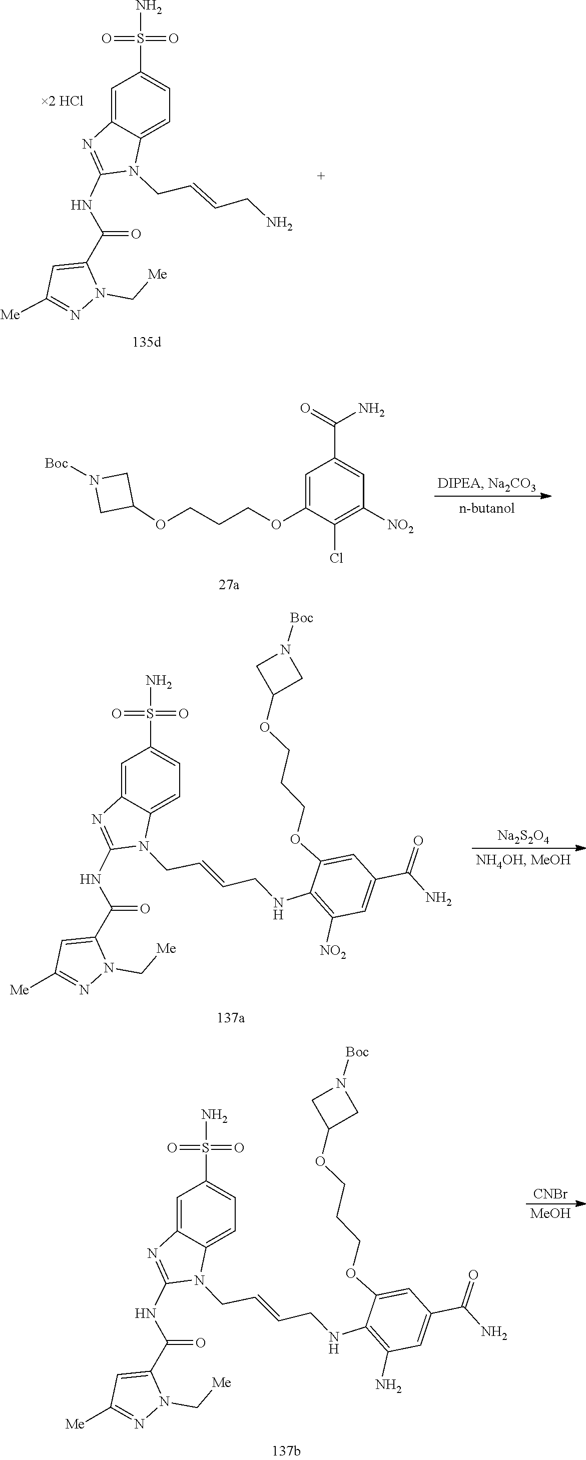

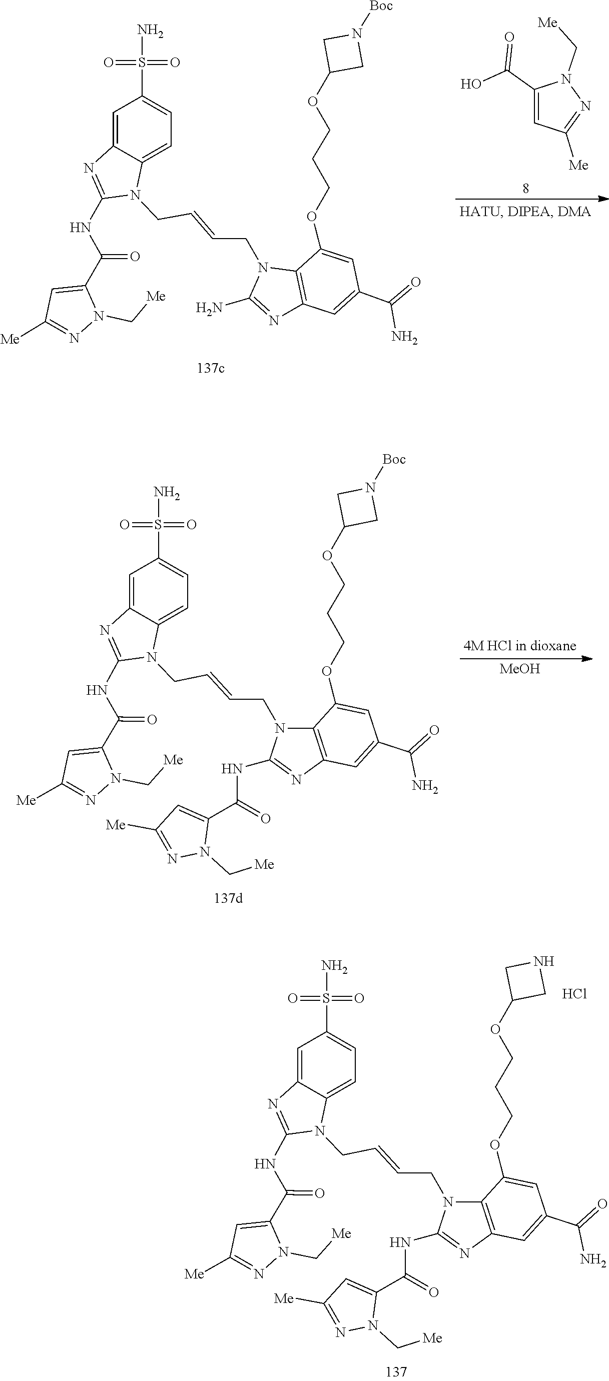

### Synthesis of 137a

Compound 137a (72 mg, 0.0893 mmol, 22% yield) was prepared using the same procedure as 135e, using 135d (200 mg, 0.408 mmol, 1 equiv.) and 27a (263 mg, 0.612 mmol, 1.5 equiv.) as the starting materials. LC-MS (Method E, ESI+): m/z [M+H]⁺=811.3 (theoretical), 811.4 (observed). HPLC retention time: 1.72 min.

### Synthesis of 137b

Compound 137b (30 mg, 0.0386 mmol, 43% yield) was prepared using the same method as 135a, using 137a (72 mg, 0.0893 mmol, 1 equiv.) as the starting material. LC-MS (Method E, ESI+): m/z [M+H]⁺=781.3 (theoretical), 781.4 (observed). HPLC retention time: 1.46 min.

### Synthesis of 137c

Compound 137c (34 mg, 0.0387 mmol, quantitative yield) was prepared using the same procedure as 135b, using 137b (30 mg, 0.03896 mmol, 1 equiv.) as the starting material. LC-MS (Method E, ESI+): m/z [M+H]⁺=806.3 (theoretical), 806.4 (observed). HPLC retention time: 1.53 min.

### Synthesis of 137d

Compound 137d (4.21 mg, 0.0036 mmol, 19% yield) was prepared using the same procedure as 135h, using 137c (17 mg, 0.0194 mmol, 1 equiv.) as the starting material. LC-MS (Method E, ESI+): m/z [M+H]⁺=942.4 (theoretical), 942.5 (observed). HPLC retention time: 1.65 min.

### Synthesis of Compound 137

Compound 137 (3.35 mg, 0.0035 mmol, quantitative yield), was prepared using the same procedure as 135, using 137d (4.21 mg, 0.0036 mmol, 1 equiv.) as the starting material. LC-MS (Method E, ESI+): m/z [M+H]⁺=842.3 (theoretical), 842.4 (observed). HPLC retention time: 1.29 min.

### Synthesis of (E)-7-(3-(azetidin-3-yloxy)propoxy)-2-(1,3-dimethyl-1H-pyrazole-5-carboxamido)-1-(4-(2-(1-ethyl-3-methyl-1H-pyrazole-5-carboxamido)-5-sulfamoyl-1H-benzo[d]imidazol-1-yl)but-2-en-1-yl-1H-benzo[d]imidazole-5-carboxamide (Compound 138)

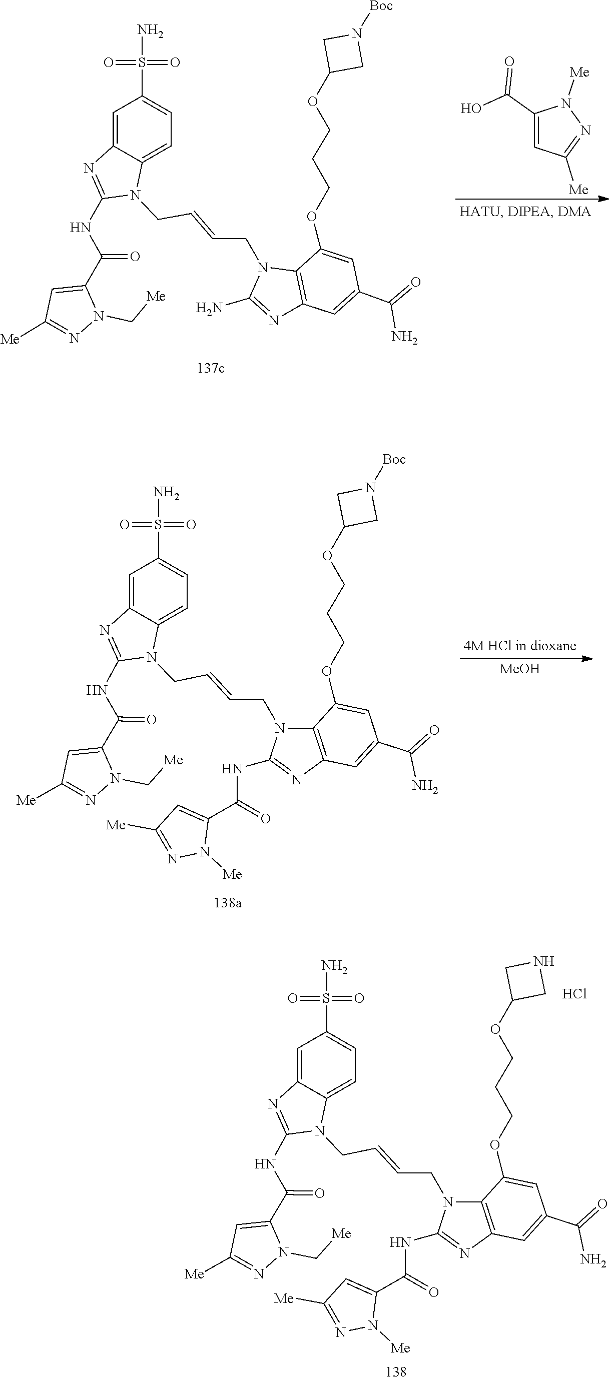

### Synthesis of 138a

Compound 138a (3.00 mg, 0.0026 mmol, 13% yield) was prepared using the same procedure as 135h, using 137c (17 mg, 0.0197 mmol, 1 equiv.) and 1, 3-dimethyl-1H-pyrazole-5-carboxyllic acid as the starting materials. LC-MS (Method E, ESI+): m/z [M+H]⁺=928.4 (theoretical), 928.5 (observed). HPLC retention time: 1.62 min.

### Synthesis of 138

Compound 138 (2.35 mg, 0.0025 mmol, quantitative yield), was prepared using the same procedure as 135, using 138a (3.00 mg, 0.0026 mmol, 1 equiv.) as the starting material. LC-MS (Method E, ESI+): m/z [M+H]⁺=828.3 (theoretical), 828.4 (observed). HPLC retention time: 1.26 min.

### Synthesis of (E)-2-(1-ethyl-3-methyl-1H-pyrazole-5-carboxamido)-1-(4-(2-(1-ethyl-3-methyl-1H-pyrazole-5-carboxamido)-5-sulfamoyl-1H-benzo[d]imidazol-1-yl)but-2-en-1-yl)-7-(3-(methylamino)propoxy)-1H-benzo[d]imidazole-5-carboxamide (Compound 139)

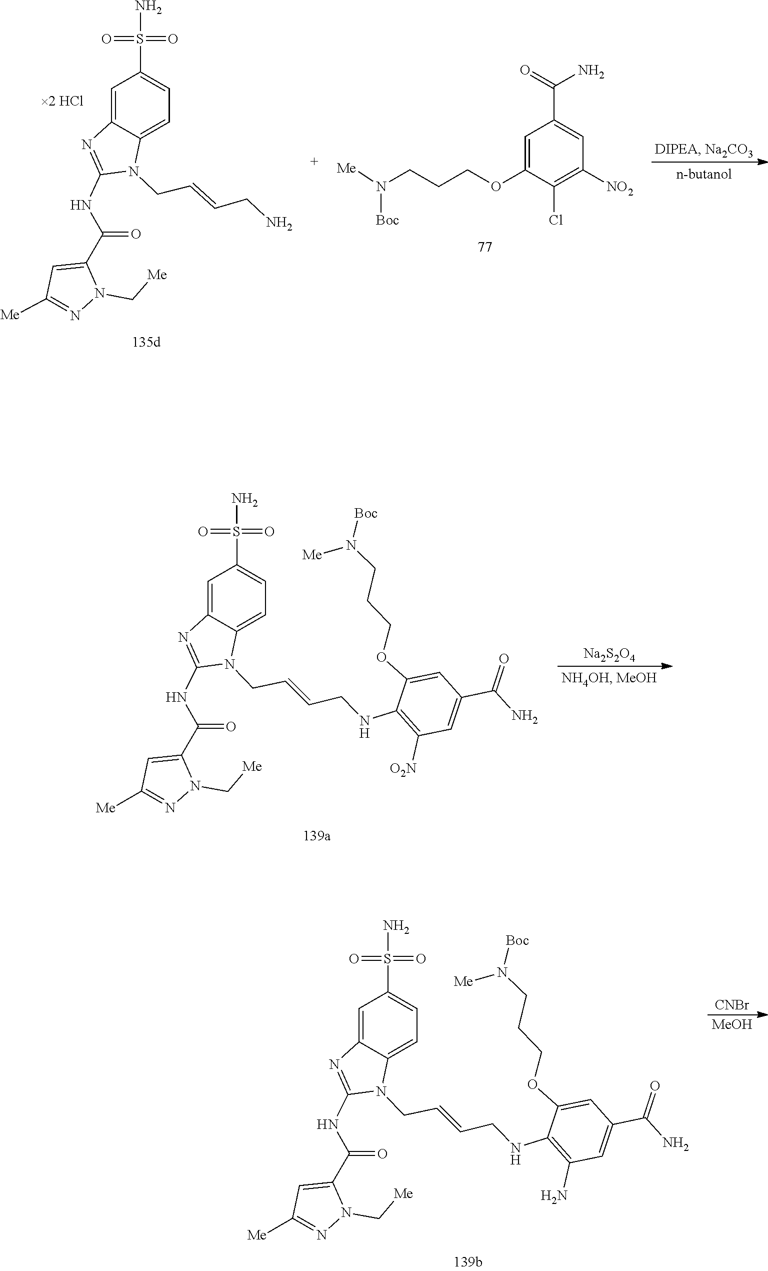

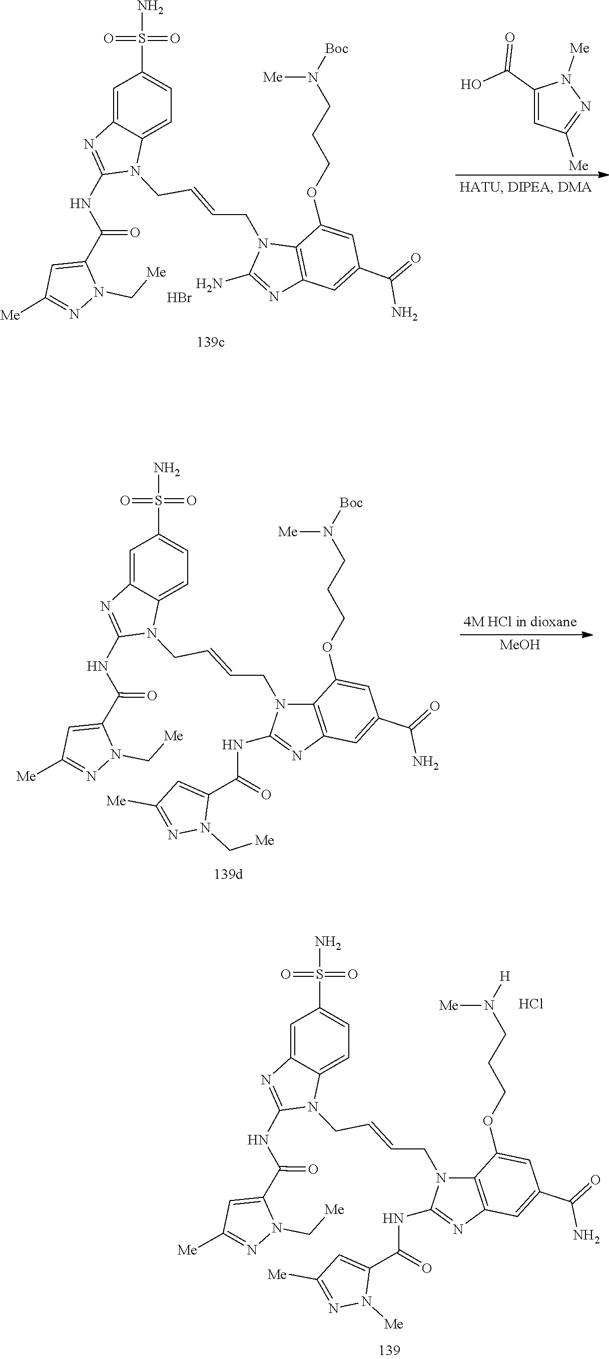

### Synthesis of 139a

Compound 139a (15 mg, 0.0686 mmol, 429% yield) was prepared using the same procedure as 135e, using 135d (1250 mg, 0.516 mmol, 1 equiv.) as the statin material LC-MS (Method E, ESI+): m/z [M+H]⁺=739.3 (theoretical), 739.4 (observed). HPLC retention time: 1.45 min.

### Synthesis of 139c

Compound 139c (57 mg, 0.0670 mmol, quantitative yield) was prepared using the same procedure as 135b, using 139b (51 mg, 0.0686 mmol, 1 equiv.) as the starting material. LC-MS (Method E, ESI+): m/z [M+H]⁺=764.3 (theoretical), 764.4 (observed). HPLC retention time: 1.31 min.

### Synthesis of 139d

Compound 139d (34 mg, 0.0303 mmol, 45% yield) was prepared following the same procedure as 135h using 139c (57 mg, 0.0670 mmol, 1 equiv.) and 1, 3-dimethyl-1H-pyrazole-5-carboxyllic acid as the starting materials. LC-MS (Method E, ESI+): m/z [M+H]⁺=886.4 (theoretical), 886.5 (observed). HPLC retention time: 1.61 min.

### Synthesis of 139

Compound 139 (27 mg, 0.0291, quantitative yield) was prepared using the same procedure as 135, using 139d (34 mg, 0.0303 mmol, 1 equiv.) as the starting material. LC-MS (Method E, ESI+): m/z [M+H]⁺=786.3 (theoretical), 786.4 (observed). HPLC retention time: 1.23 min.

### Synthesis of (E)-7-(2-(azetidin-3-yl)ethoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1,3-dimethyl-1H-pyrazole-5-carboxamido)-1H-benzo[d]imidazole-5-carboxamide (Compound 140)

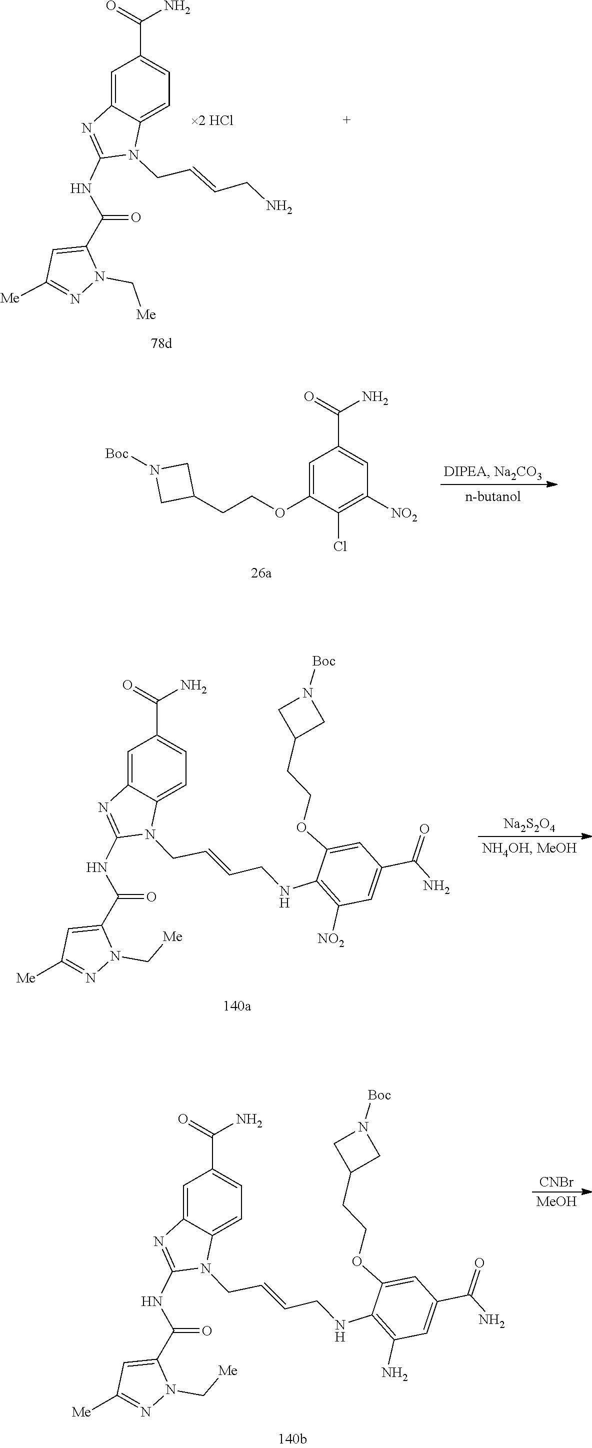

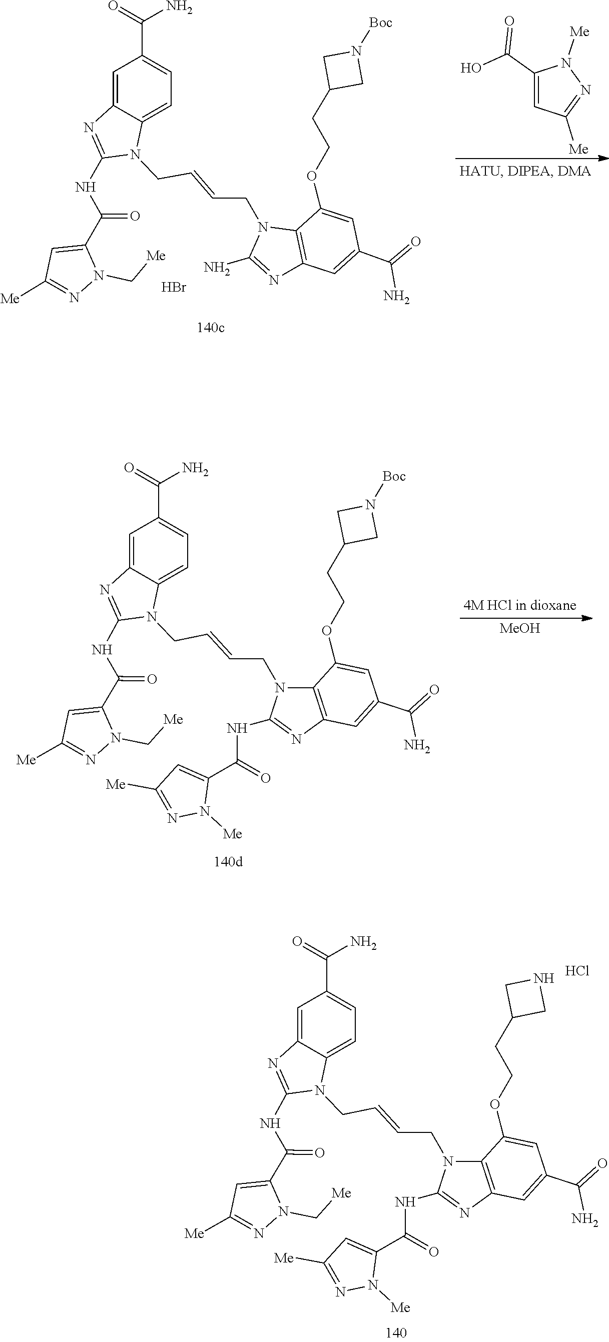

### Synthesis of 140a

Compound 140a (380 mg, 0.490 mmol, 78% yield) was prepared using the same procedure as 135e, using 26a (250 mg, 0.625 mmol, 1 equiv.) and 78d (420 mg, 0.938 mmol, 1.5 equiv.) as the starting materials. The product was precipitated in cold water and used without further purification. LC-MS (Method E, ESI+): m/z [M+H]⁺=775.3 (theoretical), 775.4 (observed). HPLC retention time: 1.66 min.

### Synthesis of 140b

Compound 140b (193 mg, 0.260 mmol, 53% yield) was prepared using the same procedure as 135a, using 140a (380 mg, 0.490 mmol, 1 equiv.) as the starting material. LC-MS (Method E, ESI+): m/z [M+H]⁺=745.4 (theoretical), 745.5 (observed). HPLC retention time: 1.44 min.

### Synthesis of 140c

Compound 140c (212 mg, 0.249 mmol, quantitative yield) was prepared using the same procedure as 135b, using 140b (193 mg, 0.260 mmol, 1 equiv.) as the starting material. LC-MS (Method E, ESI+): m/z [M+H]⁺=770.4 (theoretical), 770.5 (observed). HPLC retention time: 1.60 min.

### Synthesis of 140d

Compound 140d (38 mg, 0.0339 mmol, 27% yield) was prepared using the same procedure as 135h, using 140c (106 mg, 0.124 mmol, 1 equiv.) as the starting material. LC-MS (Method E, ESI+): m/z [M+H]⁺=892.4 (theoretical), 892.5 (observed). HPLC retention time: 1.59 min.

### Synthesis of 140

Compound 140 (30 mg, 0.0334 mmol, quantitative yield) was prepared using the same procedure as 135, using 140d (38 mg, 0.0339 mmol, 1 equiv.) as the starting material. LC-MS (Method E, ESI+): m/z [M+H]⁺=792.4 (theoretical), 792.5 (observed). HPLC retention time: 1.28 min.

### Synthesis of (E)-N-(7-(2-(azetidin-3-yl)ethoxy)-5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)-4-ethyl-2-methyloxazole-5-carboxamide (Compound 141)

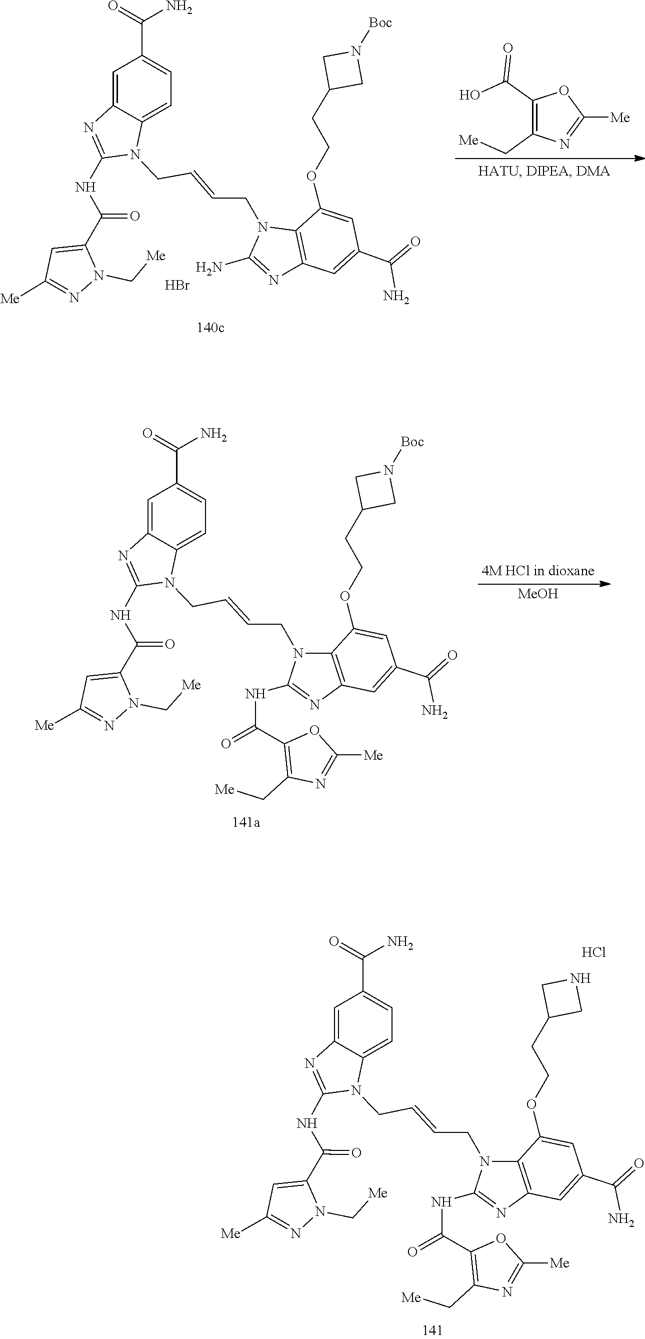

### Synthesis of 141a

Compound 141a (27 mg, 0.0237 mmol, 19% yield) was prepared using the same procedure as 135h, using 140c (106 mg, 0.124 mmol, 1 equiv.) and 4-ethyl-2-methyl-oxazole-5-carboxyllic acid as the starting materials. LC-MS (Method E, ESI+): m/z [M+H]⁺=907.4 (theoretical), 907.5 (observed). HPLC retention time: 1.57 min.

### Synthesis of 141

Compound 141 (21 mg, 0.0230 mmol, quantitative yield), was prepared using the same procedure as 135, using 141a (27 mg, 0.0237 mmol, 1 equiv.) as the starting material. LC-MS (Method E, ESI+): m/z [M+H]⁺=807.4 (theoretical), 807.5 (observed). HPLC retention time: 1.26 min.

### Synthesis of (E)-7-(3-(azetidin-3-yloxy)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1,3-dimethyl-1H-pyrazole-5-carboxamido)-1H-benzo[d]imidazole-5-carboxamide (Compound 142)

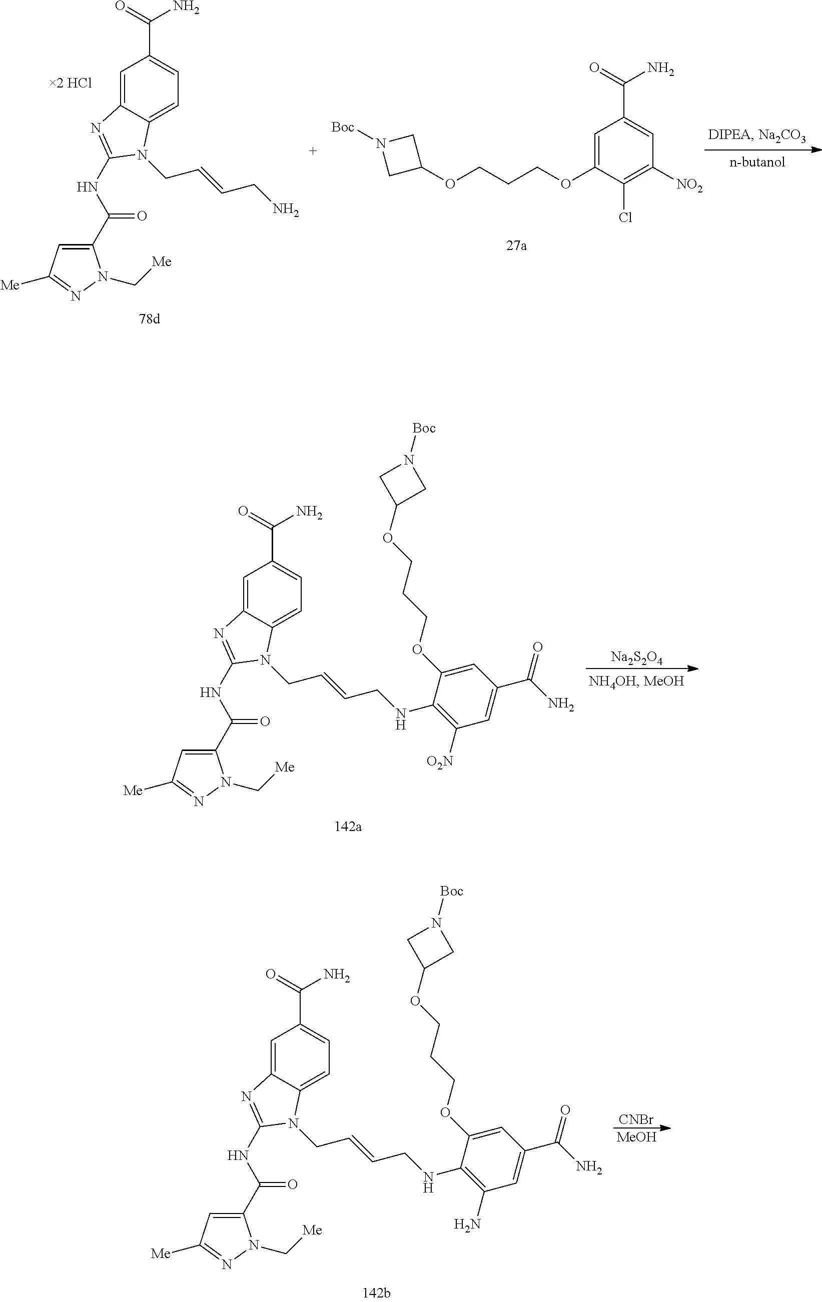

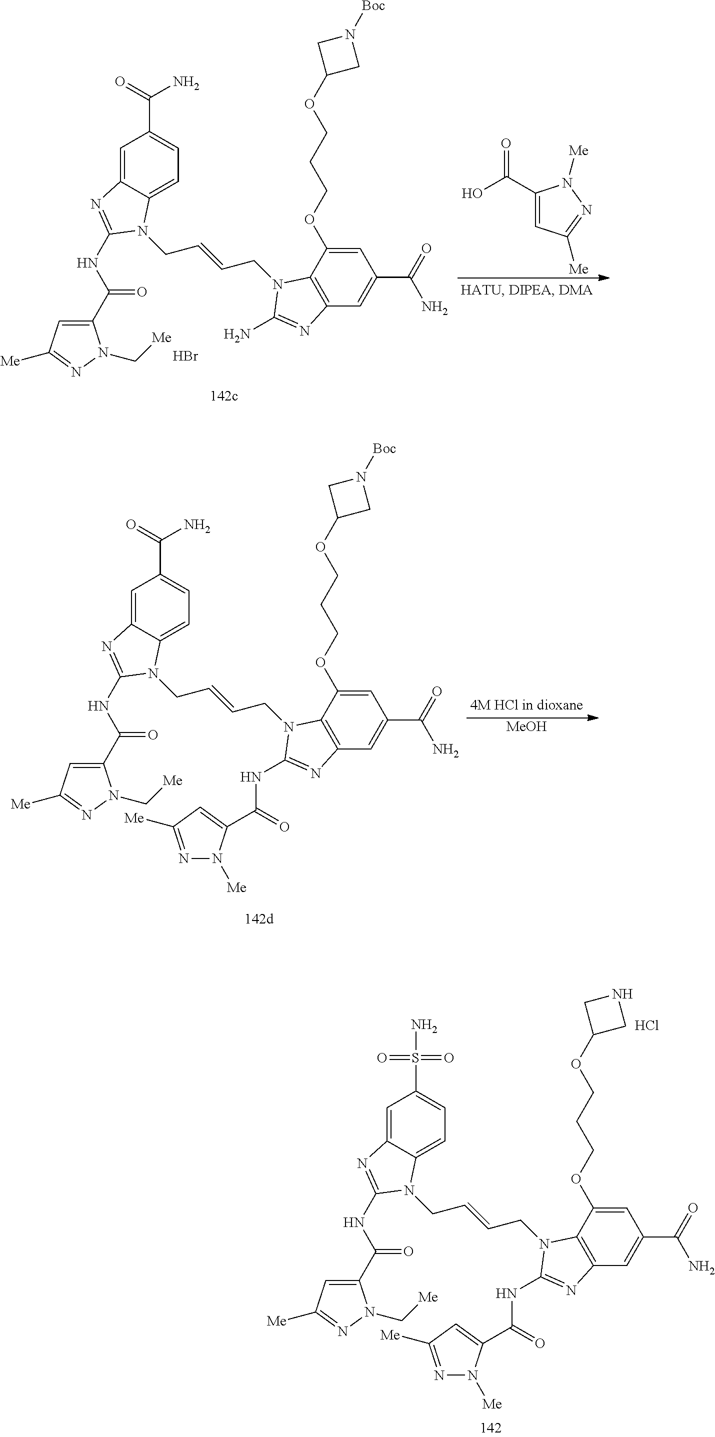

### Synthesis of 142a

Compound 142a was prepared using the same procedure as 135e, using 27a (250 mg, 0.582 mmol, 1 equiv.) and 78d (391 mg, 0.872 mmol, 1.5 equiv.) as the starting materials. The product was precipitated with cold water and used without further purification. LC-MS (Method E, ESI+): m/z [M+H]⁺=805.4 (theoretical), 805.4 (observed). HPLC retention time: 1.66 min.

### Synthesis of 142b

Compound 142b (193 mg, 0.250 mmol, 37% yield over 2 steps) was prepared using the same procedure as 135a, using 142a (548 mg, 0.681 mmol, 1 equiv.) as the starting material. LC-MS (Method E, ESI+): m/z [M+H]⁺=775.4 (theoretical), 775.5 (observed). HPLC retention time: 1.50 min.

### Synthesis of 142c

Compound 142c (164 mg, 0.186 mmol, 75% yield) was prepared using the same procedure as 135b, using 142b (193 mg, 0.260 mmol, 1 equiv.) as the starting material. LC-MS (Method E, ESI+): m/z [M+H]⁺=800.4 (theoretical), 800.5 (observed). HPLC retention time: 1.33 min.

### Synthesis of 142d

Compound 142d (40 mg, 0.0345 mmol, 37% yield) was prepared using the same procedure as 135h, using 142c (48 mg, 0.373 mmol, 1 equiv.) as the starting material. LC-MS (Method E, ESI+): m/z [M+H]⁺=922.4 (theoretical), 922.5 (observed). HPLC retention time: 1.58 min.

### Synthesis of 142

Compound 142 (32 mg, 0.0323 mmol, quantitative yield) was prepared using the same procedure as 135, using 142d (40 mg, 0.0345 mmol, 1 equiv.) as the starting material. LC-MS (Method E, ESI+): m/z [M+H]⁺=822.4 (theoretical), 822.5 (observed). HPLC retention time: 1.29 min.

### Synthesis of (E)-N-(7-(3-(azetidin-3-yloxy)propoxy)-5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)-4-ethyl-2-methyloxazole-5-carboxamide (Compound 143)

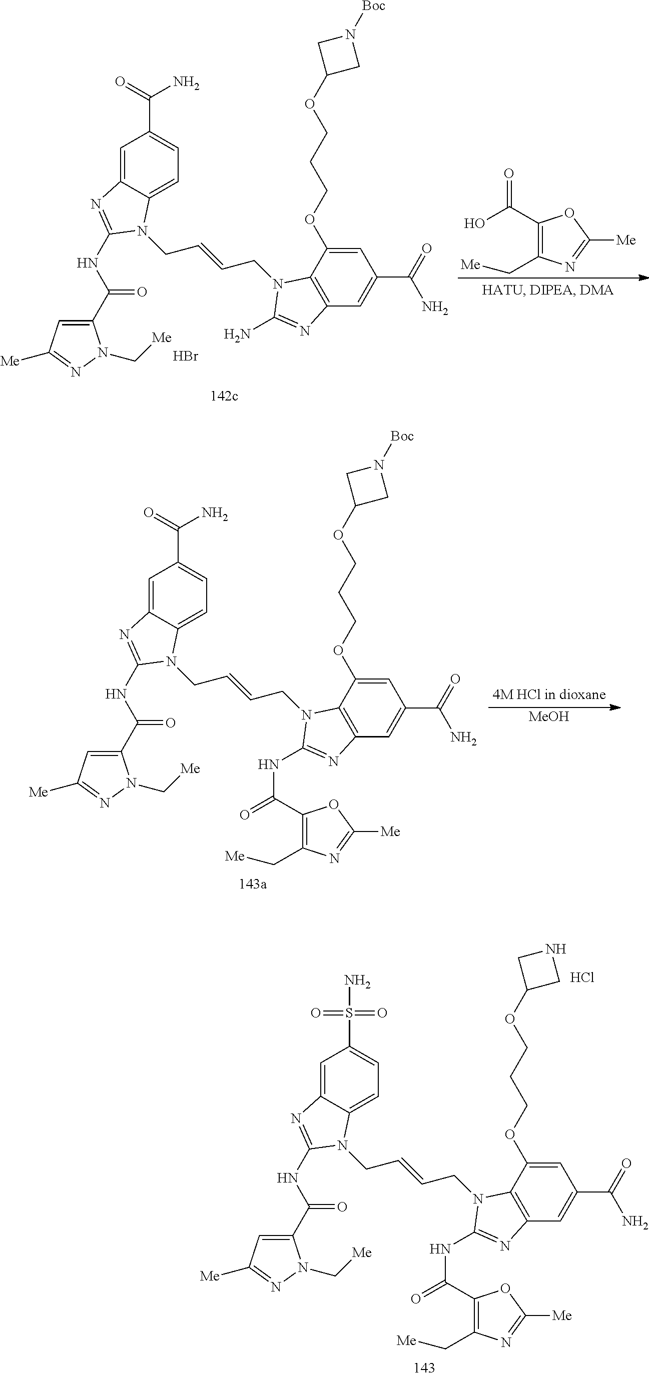

### Synthesis of 143a

Compound 143a (31 mg, 0.0263 mmol, 28% yield) was prepared using the same procedure as 135h, using 142c (82 mg, 0.0.0931 mmol, 1 equiv.) and 4-ethyl-2-methyl-oxazole-5-carboxyllic acid as the starting materials. LC-MS (Method E, ESI+): m/z [M+H]⁺=937.4 (theoretical), 937.5 (observed). HPLC retention time: 1.56 min.

### Synthesis of 143

Compound 143 (25 mg, 0.0261 mmol, quantitative yield), was prepared using the same procedure as 135, using 141a (31 mg, 0.0263 mmol, 1 equiv.) as the starting material. LC-MS (Method E, ESI+): m/z [M+H]⁺=837.4 (theoretical), 837.5 (observed). HPLC retention time: 1.29 min.

### Synthesis of (E)-7-(2-(1-(3-(2,5-dioxo-2,5-dihydro-1H-pyrrol-1-yl)propanoyl)azetidin-3-yl)ethoxy)-2-(1-ethyl-3-methyl-1H-pyrazole-5-carboxamido)-1-(4-(2-(1-ethyl-3-methyl-1H-pyrazole-5-carboxamido)-5-sulfamoyl-1H-benzo[d]imidazol-1-yl)but-2-en-1-yl)-1H-benzo[d]imidazole-5-carboxamide (Compound 144)

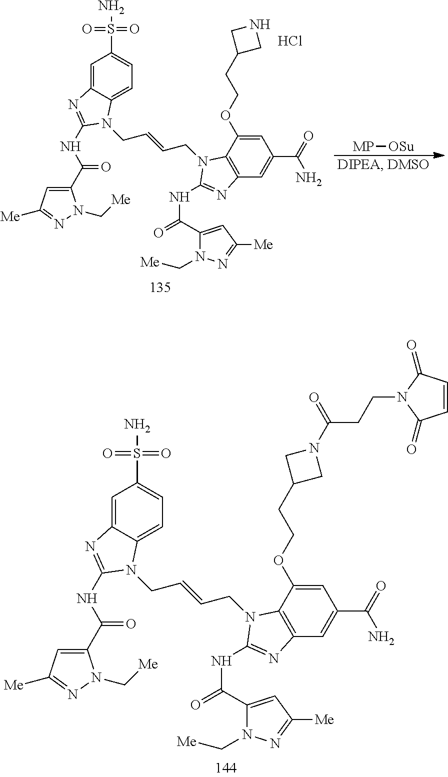

The ×2 TFA salt of compound 144 (0.59 mg, 0.0005 mmol, 24% yield) was prepared according to general method 9, using compound 135 (1.86 mg, 0.0020 mmol, 1 equiv.) as the starting material. LC-MS (Method E, ESI+): m/z [M+H]⁺=963.4 (theoretical), 963.5 (observed). HPLC retention time: 1.44 min.

### Synthesis of (E)-2-(1,3-dimethyl-1H-pyrazole-5-carboxamido)-7-(2-(1-(3-(2,5-dioxo-2,5-dihydro-1H-pyrrol-1-yl)propanoyl)azetidin-3-yl)ethoxy)-1-(4-(2-(1-ethyl-3-methyl-1H-pyrazole-5-carboxamido)-5-sulfamoyl-1H-benzo[d]imidazol-1-yl)but-2-en-1-yl)-1H-benzo[d]imidazole-5-carboxamide (Compound 145)

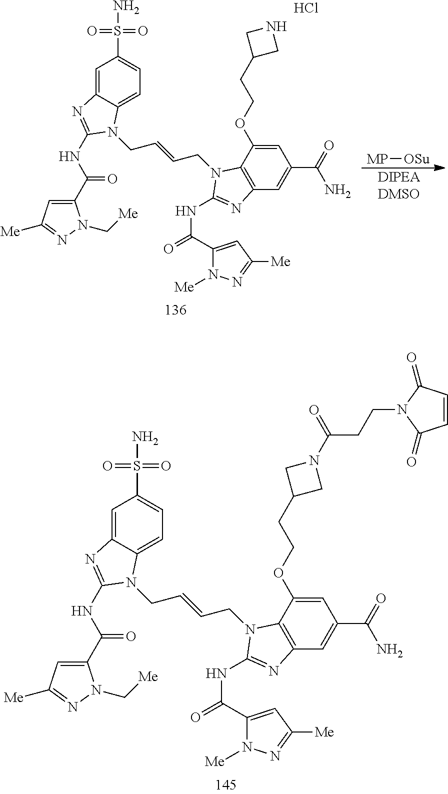

The ×2 TFA salt of compound 145 (0.31 mg, 0.0003 mmol, 12% yield) was prepared according to general method 9, using compound 136 (2.09 mg, 0.0023 mmol, 1 equiv.) as the starting material. LC-MS (Method E, ESI+): m/z [M+H]⁺=949.3 (theoretical), 949.5 (observed). HPLC retention time: 1.40 min.

### Synthesis of (E)-7-(3-((1-(3-(2,5-dioxo-2,5-dihydro-1H-pyrrol-1-yl)propanoyl)azetidin-3-yl)oxy)propoxy)-2-(1-ethyl-3-methyl-1H-pyrazole-5-carboxamido)-1-(4-(2-(1-ethyl-3-methyl-1H-pyrazole-5-carboxamido)-5-sulfamoyl-1H-benzo[d]imidazol-1-yl)but-2-en-1-yl)-1H-benzo[d]imidazole-5-carboxamide (Compound 146)

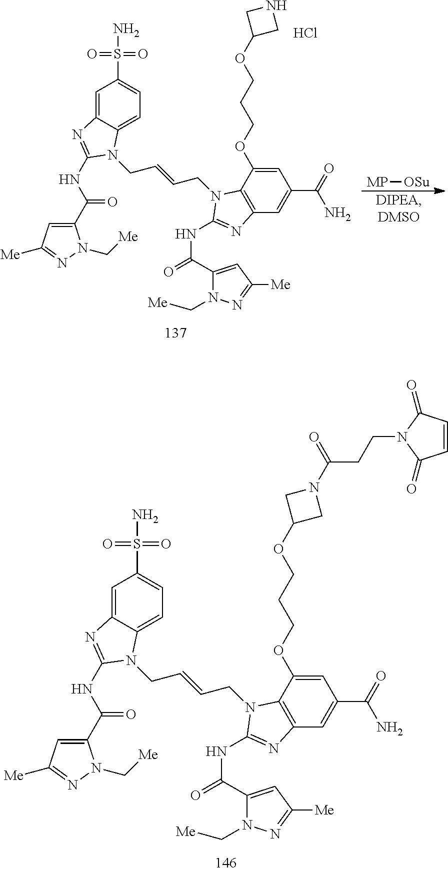

The ×2 TFA salt of compound 146 (0.92 mg, 0.0008 mmol, 21% yield) was prepared according to general method 9, using compound 137 (3.35 mg, 0.0035 mmol, 1 equiv.) as the starting material. LC-MS (Method E, ESI+): m/z [M+H]⁺=993.4 (theoretical), 993.5 (observed). HPLC retention time: 1.43 min.

### Synthesis of (E)-2-(1,3-dimethyl-1H-pyrazole-5-carboxamido)-7-(3-((1-(3-(2,5-dioxo-2,5-dihydro-1H-pyrrol-1-yl)propanoyl)azetidin-3-yl)oxy)propoxy)-1-(4-(2-(1-ethyl-3-methyl-1H-pyrazole-5-carboxamido)-5-sulfamoyl-1H-benzo[d]imidazol-1-yl)but-2-en-1-yl)-1H-benzo[d]imidazole-5-carboxamide (Compound 147)

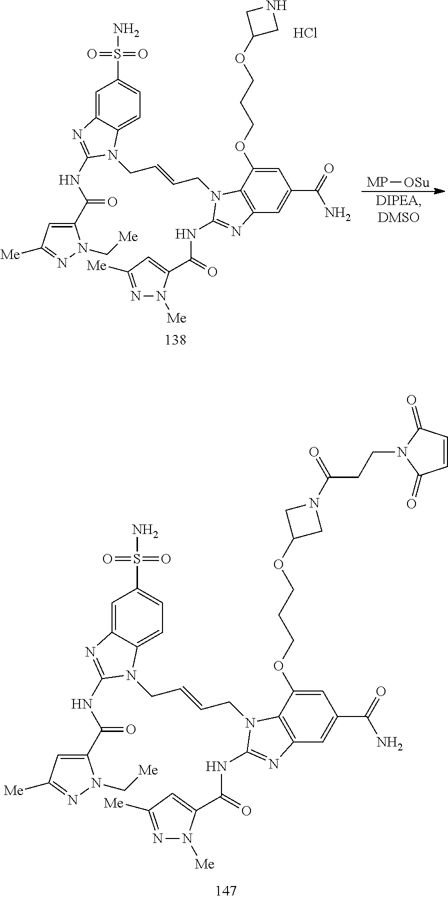

The ×2 TFA salt of compound 147 (0.36 mg, 0.0003 mmol, 12% yield) was prepared according to general method 9, using compound 136 (2.83 mg, 0.0024 mmol, 1 equiv.) as the starting material. LC-MS (Method E, ESI+): m/z [M+H]⁺=979.4 (theoretical), 979.5 (observed). HPLC retention time: 1.41 min.

### Synthesis of (E)-2-(1,3-dimethyl-1H-pyrazole-5-carboxamido)-7-(3-(3-(2,5-dioxo-2,5-dihydro-1H-pyrrol-1-yl)-N-methylpropanamido)propoxy)-1-(4-(2-(1-ethyl-3-methyl-1H-pyrazole-5-carboxamido)-5-sulfamoyl-1H-benzo[d]imidazol-1-yl)but-2-en-1-yl)-1H-benzo[d]imidazole-5-carboxamide (Compound 148)

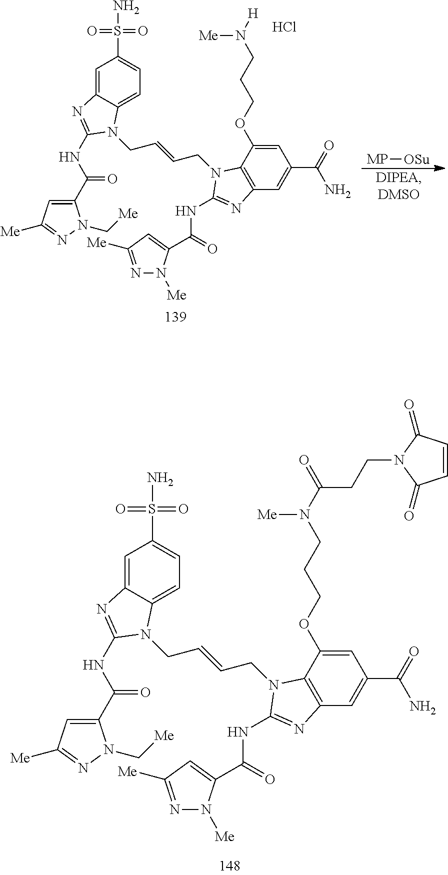

The ×2 TFA salt of compound 148 (15 mg, 0.0129 mmol, 44% yield) was prepared according to general method 9, using compound 139 as the starting material. LC-MS (Method E, ESI+): m/z [M+H]⁺=937.3 (theoretical), 937.4 (observed). HPLC retention time: 1.42 min.

### Synthesis of (E)-1-(4-(5-carbamoyl-2-(1,3-dimethyl-1H-pyrazole-5-carboxamido)-7-(2-(1-(3-(2,5-dioxo-2,5-dihydro-1H-pyrrol-1-yl)propanoyl)azetidin-3-yl)ethoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide (Compound 149)

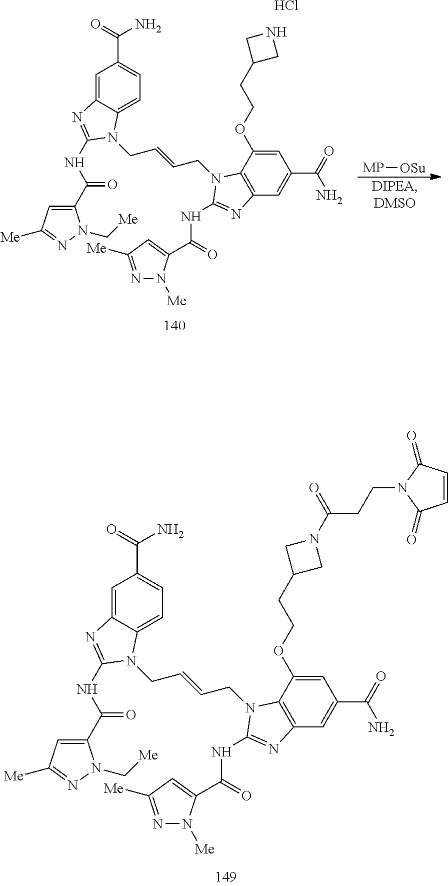

The ×2 TFA salt of compound 149 (16 mg, 0.0136 mmol, 41% yield) was prepared according to general method 9, using compound 140 (30 mg, 0.0334 mmol, 1 equiv). as the starting material. LC-MS (Method E, ESI+): m/z [M+H]⁺=943.4 (theoretical), 943.5 (observed). HPLC retention time: 1.41 min.

### Synthesis of (E)-N-(5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-7-(2-(1-(3-(2,5-dioxo-2,5-dihydro-1H-pyrrol-1-yl)propanoyl)azetidin-3-yl)ethoxy)-1H-benzo[d]imidazol-2-yl)-4-ethyl-2-methyloxazole-5-carboxamide (Compound 150)

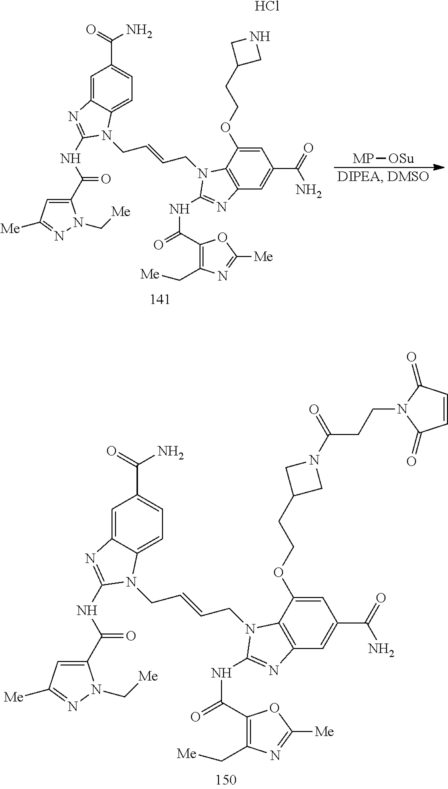

The ×2 TFA salt of compound 150 (15 mg, 0.0123 mmol, 53% yield) was prepared according to general method 9, using compound 141 (21 mg, 0.0230 mmol, 1 equiv.) as the starting material. LC-MS (Method E, ESI+): m/z [M+H]⁺=943.4 (theoretical), 943.5 (observed). HPLC retention time: 1.41 min.

### Synthesis of (E)-1-(4-(5-carbamoyl-2-(1,3-dimethyl-1H-pyrazole-5-carboxamido)-7-(3-((1-(3-(2,5-dioxo-2,5-dihydro-1H-pyrrol-1-yl)propanoyl)azetidin-3-yl)oxy)propoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide (Compound 151)

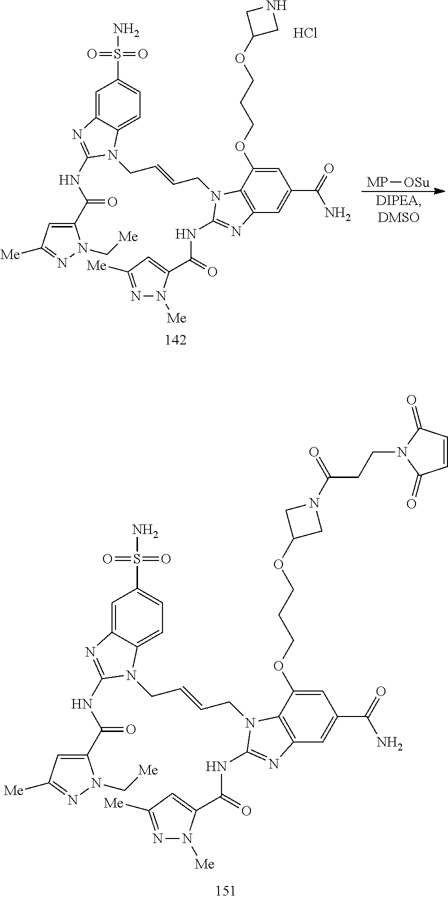

The ×2 TFA salt of compound 151 (22 mg, 0.0182 mmol, 56% yield) was prepared according to general method 9, using compound 142 (30 mg, 0.0323 mmol, 1 equiv.) as the starting material. LC-MS (Method E, ESI+): m/z [M+H]⁺=973.4 (theoretical), 973.5 (observed). HPLC retention time: 1.42 min.

### Synthesis of (E)-N-(5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-7-(3-((1-(3-(2,5-dioxo-2,5-dihydro-1H-pyrrol-1-yl)propanoyl)azetidin-3-yl)oxy)propoxy)-1H-benzo[d]imidazol-2-yl)-4-ethyl-2-methyloxazole-5-carboxamide (Compound 152)

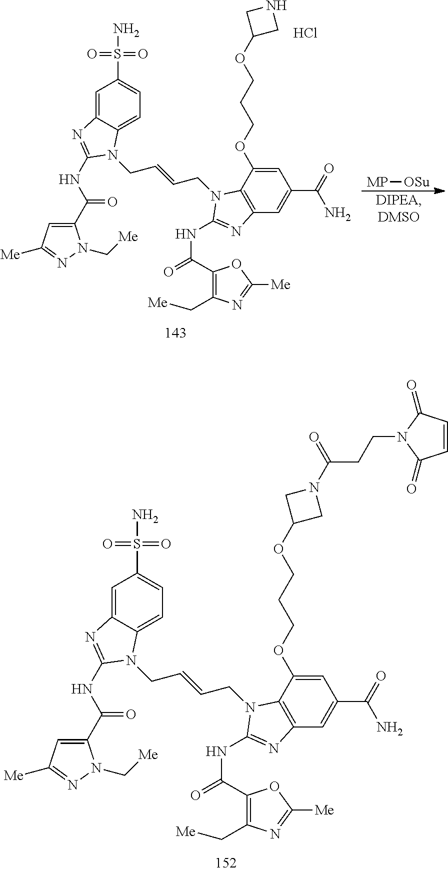

The ×2 TFA salt of compound 152 (20 mg, 0.0168 mmol, 43% yield) was prepared according to general method 9, using compound 143 (37 mg, 0.0388 mmol, 1 equiv.) as the starting material. LC-MS (Method E, ESI+): m/z [M+H]⁺=988.4 (theoretical), 988.5 (observed). HPLC retention time: 1.40 min.

### Synthesis of S-(1-(3-(3-(2-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1,3-dimethyl-1H-pyrazole-5-carboxamido)-1H-benzo[d]imidazol-7-yl)oxy)ethyl)azetidin-1-yl)-3-oxopropyl)-2,5-dioxopyrrolidin-3-yl)-L-cysteine (Compound 153)

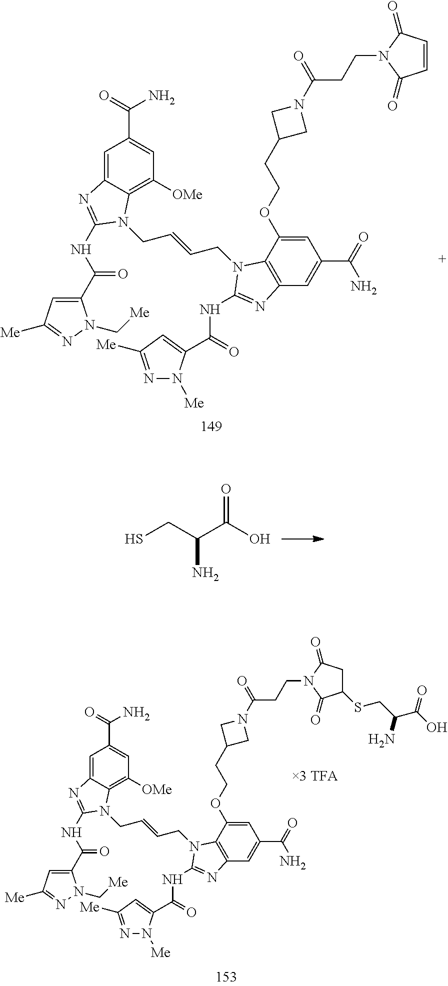 [SMILES: CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCC3CN(C(=O)CCN4C(=O)C=CC4=O)C3)c21 ; N[C@@H](CS)C(=O)O ; CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCC3CN(C(=O)CCN4C(=O)CC(SC[C@H](N)C(=O)O)C4=O)C3)c21]

To a solution of compound 149 (10 mM in DMSO, 0.42 mL, 0.0042 mmol, 1 equiv.) was added 1-cysteine (0.1 M H₂O, 63 μL, 0.063 mmol, 1.5 equiv.). The reaction stirred at 30° C. for 1 h and was monitored by UPLC-MS. Upon completion, the reaction mixture was purified directly by preparatory HPLC (method G). Pure fractions were collected, frozen, and lyophilized to yield compound 153 (2.17 mg, 0.0015 mmol, 36% yield). LC-MS (Method E, ESI+): m/z [M+H]⁺=1079.4 (theoretical), 1079.5 (observed). HPLC retention time: 1.28 min.

### Synthesis of S-(1-(3-(3-(2-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(4-ethyl-2-methyloxazole-5-carboxamido)-1H-benzo[d]imidazol-7-yl)oxy)ethyl)azetidin-1-yl)-3-oxopropyl)-2,5-dioxopyrrolidin-3-yl)-L-cysteine (Compound 154)

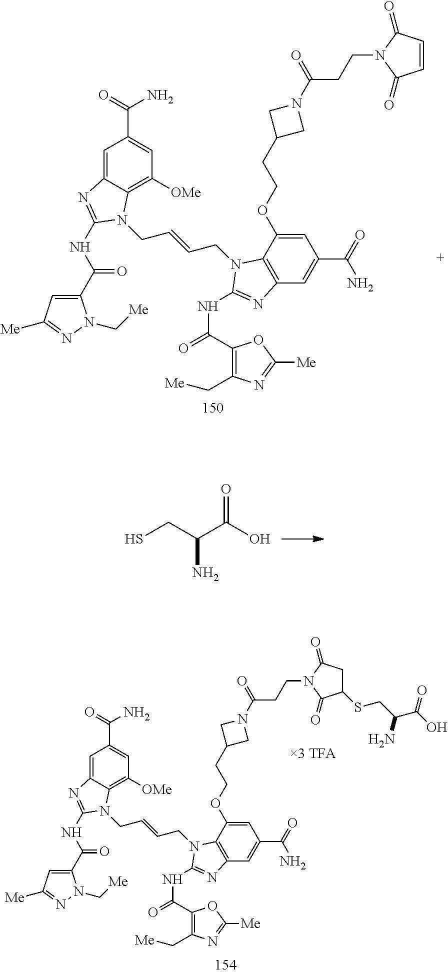 [SMILES: CCc1nc(C)oc1C(=O)Nc1nc2cc(C(N)=O)cc(OCCC3CN(C(=O)CCN4C(=O)C=CC4=O)C3)c2n1C/C=C/Cn1c(NC(=O)c2cc(C)nn2CC)nc2cc(C(N)=O)cc(OC)c21 ; N[C@@H](CS)C(=O)O ; CCc1nc(C)oc1C(=O)Nc1nc2cc(C(N)=O)cc(OCCC3CN(C(=O)CCN4C(=O)CC(SC[C@H](N)C(=O)O)C4=O)C3)c2n1C/C=C/Cn1c(NC(=O)c2cc(C)nn2CC)nc2cc(C(N)=O)cc(OC)c21]

Compound 150 (2.35 mg, 0.0017 mmol, 39% yield) was prepared using the same procedure as compound 153, using compound 145 (10 mM in DMSO, 0.43 mL, 0.0043 mmol, 1 equiv.) as the starting material. LC-MS (Method E, ESI+): m/z [M+H]⁺=1064.4 (theoretical), 1064.5 (observed). HPLC retention time: 1.29 min.

### Synthesis of S-(1-(3-(3-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1,3-dimethyl-1H-pyrazole-5-carboxamido)-1H-benzo[d]imidazol-7-yl)oxy)propoxy)azetidin-1-yl)-3-oxopropyl)-2,5-dioxopyrrolidin-3-yl)-L-cysteine (Compound 155)

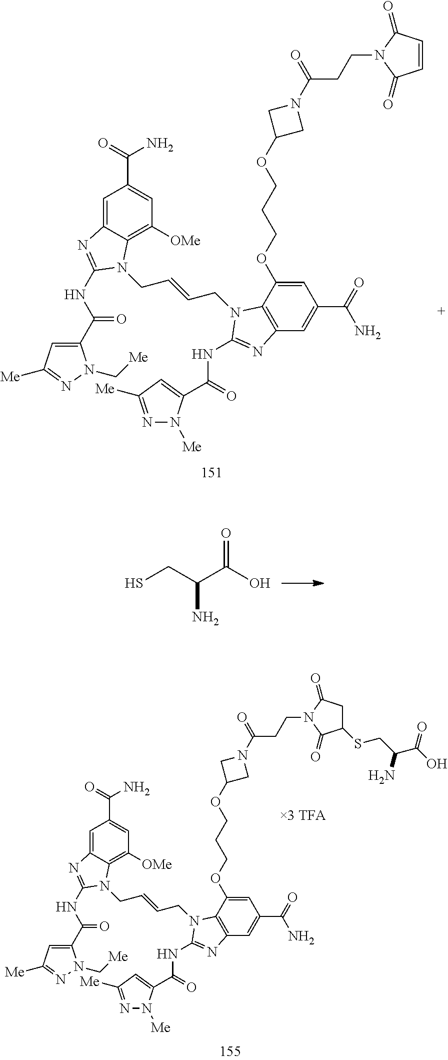 [SMILES: CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCCOC3CN(C(=O)CCN4C(=O)C=CC4=O)C3)c21 ; N[C@@H](CS)C(=O)O ; CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCCOC3CN(C(=O)CCN4C(=O)CC(SC[C@H](N)C(=O)O)C4=O)C3)c21]

Compound 155 (2.34 mg, 0.0016 mmol, 39% yield) was prepared using the same procedure as compound 153, using compound 151 (10 mM in DMSO, 0.41 mL, 0.0041 mmol, 1 equiv.) as the starting material. LC-MS (Method E, ESI+): m/z [M+H]⁺=1109.4 (theoretical), 1109.5 (observed). HPLC retention time: 1.30 min.

### Synthesis of S-(1-(3-(3-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(4-ethyl-2-methyloxazole-5-carboxamido)-1H-benzo[d]imidazol-7-yl)oxy)propoxy)azetidin-1-yl)-3-oxopropyl)-2,5-dioxopyrrolidin-3-yl)-L-cysteine (Compound 156)

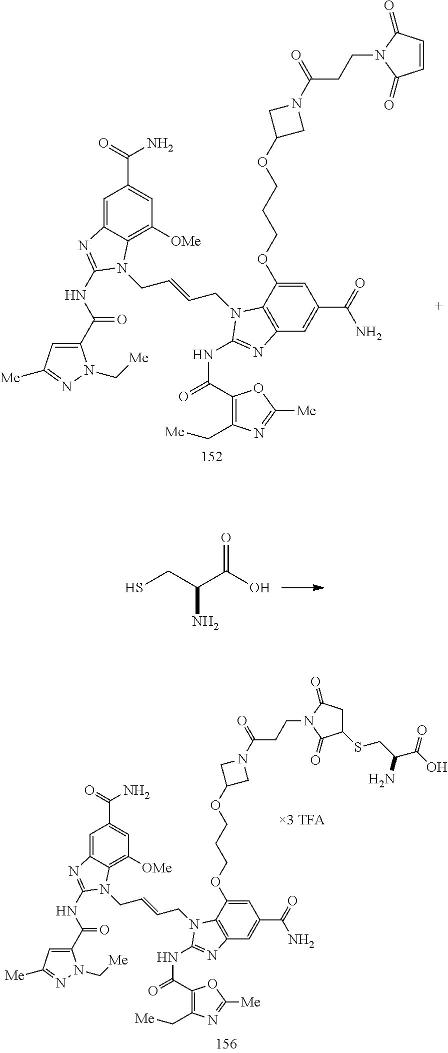 [SMILES: CCc1nc(C)oc1C(=O)Nc1nc2cc(C(N)=O)cc(OCCCOC3CN(C(=O)CCN4C(=O)C=CC4=O)C3)c2n1C/C=C/Cn1c(NC(=O)c2cc(C)nn2CC)nc2cc(C(N)=O)cc(OC)c21 ; N[C@@H](CS)C(=O)O ; CCc1nc(C)oc1C(=O)Nc1nc2cc(C(N)=O)cc(OCCCOC3CN(C(=O)CCN4C(=O)CC(SC[C@H](N)C(=O)O)C4=O)C3)c2n1C/C=C/Cn1c(NC(=O)c2cc(C)nn2CC)nc2cc(C(N)=O)cc(OC)c21]

Compound 156 (2.31 mg, 0.0016 mmol, 39% yield) was prepared using the same procedure as compound 153, using compound 152 (10 in mM DMSO, 0.42 mL, 0.0042 mmol, 1 equiv.) as the starting material. LC-MS (Method E, ESI+): m/z [M+H]⁺=1094.4 (theoretical), 1094.5 (observed). HPLC retention time: 1.32 min.

### General Procedures for the Preparation of ADCs:

ADCs were prepared as described previously (Methods Enzymol. 2012, 502, 123-138). Briefly, DAR (drug-to-antibody ratio) 4 conjugates were prepared by partial reduction of the antibody inter-chain disulfide bonds using a sub-stoichiometric amount of tris(2-carboxyethyl)phosphine (TCEP). TCEP was added at approximately 2.2 molar equivalents relative to the antibody (TCEP:antibody) to a pre-warmed (37° C.) antibody stock solution in phosphate buffered saline, (PBS, Gibco, PN 10010023) and 1 M EDTA. The reduction reaction mixture was incubated at 37° C. for approximately 60 minutes. Conjugation of the partially-reduced antibody with maleimide drug-linker was carried out by adding 6 molar equivalents of the drug-linker as a DMSO stock solution. Additional DMSO was added as necessary to achieve a final reaction concentration of 10% (v/v) DMSO to keep the drug-linker remain in solution during the conjugation reaction. The conjugation reaction was allowed to proceed for 30 minutes at room temperature or until all available antibody cysteine thiols had been alkylated by drug-linker as indicated by reversed-phase HPLC (Method G). Removal of excess drug-linker was achieved by incubating the reaction mixture with 100% molar excess QuadraSil® MP resin (Millipore Sigma, PN 679526) for 30 minutes at room temperature. Buffer exchange into formulation buffer (PBS, Gibco, PN 10010023) was achieved by gel filtration chromatography using a prepacked PD-10 column (GE Life Sciences, PN 17043501) according to manufacturer's instructions. Further removal of residual drug-linker was achieved by repeated diafiltration (5-10 times) of the reaction mixture containing the ADCs in formulation buffer using a 30 kilodalton molecular weight cutoff centrifugal filter (Millipore Sigma, PN Z717185), until there was no detectable free drug-linker remaining, as indicated by HPLC analysis (Method K).

### General Procedures for the Characterization of ADCs:

ADCs were characterized using the following methods:

Method I: Size-exclusion chromatography (SEC) was performed with a Waters ACQUITY UPLC system and an Acquity UPLC Protein BEH SEC Column, (200 Å, 1.7 μm, 4.6×150 mm, PN: 186005225). The mobile phase used was 7.5% isopropanol in 92.5% aqueous (25 mM sodium phosphate, 350 mM NaCl, pH 6.8), v/v. Elution was performed isocratically at a flow rate of 0.4 mL/min at ambient temperature.

Method J: Reversed-phase chromatography (RP-HPLC) was performed on a Waters 2695 HPLC system and an Agilent PLRP-S column (1000 Å, 8 μm 50×2.1 mm, PN: PL1912-1802). ADCs were treated with 10 mM DTT to reduce disulfide bonds prior to analysis. Sample elution was done using Mobile Phase A (0.05% (v/v) TFA in water) and Mobile Phase B (0.01% (v/v) TFA in MeCN) with a gradient of 25-44% B over 12.5 minutes at 80° C. The drug-to-antibody ratio (DAR) was calculated based on the integrated peak area measured at UV 280 nm.

### Calculations of Molar Ratios

The average drug loading per antibody light-chain (MR_{DLC}) or antibody heavy-chain (MR_{DHC}) was calculated using the equations below:

$$M {R}_{D L C} = \frac{\sum ( LC \% {area}_{n} \times {MR}_{n} )}{100}$$

where MR_{DLC}=average drug-to-light chain ratio
   LC % areaₙ=% area of the nth loaded light chain species
      % areas based on light chain peaks only
   MRₙ=drug-to-antibody ratio of the nth loaded species
AND

$${MR}_{DHC} = \frac{\sum ( HC \% {area}_{n} \times {MR}_{n} )}{100}$$

where MR_{DHC}=average drug-to-heavy chain ratio
   HC % areaₙ=% area of the nth loaded heavy chain species
      % areas based on heavy chain peaks only
   MRₙ=drug-to-antibody ratio of the nth loaded species

The average drug loading per antibody (MR_{D}) was calculated using the equation below:

MR_{D}=2×(MR_{DLC}+MR_{DHC})
where MR_{D}=average drug-to-antibody ratio
   MR_{DLC}=average drug-to-light chain ratio
   MR_{DHC}=average drug-to-heavy chain ratio

Method K: Residual unconjugated drug linker was measured on a Waters ACQUITY UPLC system using an ACQUITY UPLC BEH C18 Column (130 Å, 1.7 μm, 2.1 mm×50 mm, PN: 186002350). ADC samples were treated with 2× volumes of ice-cold MeOH to induce precipitation and pelleted by centrifugation. The supernatant, containing any residual, unconjugated drug-linker, was injected onto the system. Sample elution was done using Mobile Phase A (0.05% (v/v) TFA in Water) and Mobile Phase B (0.01% TFA (v/v) in MeCN) with a gradient of 1-95% B over 2 minutes at 50° C. Detection was performed at 215 nm and quantitation of the residual drug-linker compound was achieved using an external standard of the corresponding linker.

### Example 2

### In Vitro Potency Evaluation of Sting Agonists and Corresponding ADCS

### Experimental Procedures of In Vitro Biological Assays

THP1-Dual™ Cell Reporter Assay

Potency of compounds and ADCs was evaluated using the THP1-Dual™ cells (InvivoGen PN: thpd-nfis [also referred to as THP1 dual reporter cells]), which contain an IRF-Lucia luciferase reporter. Cells were cultured in RPMI-1640 (Gibco) with 10% heat-inactivated fetal bovine serum, Pen-Strep (100 U/mL-100 μg/mL, Gibco), HEPES (10 mM, Gibco)), sodium pyruvate (1 mM, Gibco), MEM non-essential amino acids (1×, Gibco), GlutaMAX (1×, Gibco), and beta-mercaptoethanol (55 μM, Gibco). Cells were plated in a 96-well flat bottom tissue culture-treated clear polystyrene plate (Corning Costar #3596) at ˜100,000 cells per well in 200 μL with the indicated concentration of the compound or ADC. The supernatant was harvested at 24 hours (compounds) or 48 hours (ADC) post plating for the reporter assay, or as indicated. To measure Lucia reporter signal, 10 μL of the supernatant was combined with 40 μL of QUANTI-Luc™ Luminescence assay reagent (Invivogen PN: rep-qlc1) in a 96-well clear flat bottom tissue culture-treated black polystyrene plate (Corning Costar #3603) and read on a Perkin Elmer Envision plate reader.

Bone Marrow-Derived Macrophage Assay

Potency of the compounds described herein was evaluated using mouse bone-marrow derived macrophages cultured from wild type (C57BL/6J, the Jackson Laboratory #000664) or STING-deficient (C57BL/6J-Sting1^{gt}/J, the Jackson Laboratory #017537) mice. Briefly, mouse bone marrow cells were cultured for 7-10 days in RPMI-1640 (Gibco) with 10% heat-inactivated fetal bovine serum, Pen-Strep (100 U/mL-100 μg/mL, Gibco), HEPES (10 mM, Gibco)), sodium pyruvate (1 mM, Gibco), GlutaMAX (1×, Gibco), beta-mercaptoethanol (55 μM, Gibco) and 20-40 ng/mL murine M-CSF (Peprotech, #315-02). Cells were plated in a 96-well flat bottom tissue culture-treated clear polystyrene plate (Corning Costar #3596) at ˜100,000 cells per well in 200 μL with the indicated concentration of the compound. The supernatant was harvested at 24 hours and cytokines were measured using a Milliplex MAP mouse cytokine/chemokine magnetic bead panel assay kit (MCYTOMAG-70k custom 11-plex kit: MCP1, MIP1α, MIP1β, TNFα, IFNγ, IL-10, IL-12p70, IL-1β, IL-6, IP10, RANTES) and analyzed using a Luminex™ MAGPIX™ Instrument System.

Bystander Activity Assay

Bystander activity of ADCs was evaluated using Renca cancer cells and THP1-Dual™ cells (InvivoGen) which contain an IRF-Lucia luciferase reporter. Cells were cultured in RPMI-1640 (Gibco) with 10% heat-inactivated fetal bovine serum, Pen-Strep (100 U/ml-100 μg/ml, Gibco), HEPES (10 mM, Gibco), sodium pyruvate (1 mM, Gibco), MEM non-essential amino acids (1×, Gibco), GlutaMAX (1×, Gibco), and beta-mercaptoethanol (55 μM, Gibco). Renca cells were plated in a 96-well flat bottom tissue culture-treated clear polystyrene plate (Corning Costar #3596) at 50,000 cells per well in 100 μL. On the day following the initial plating, 50,000 THP1-Dual™ cells were added to each well with the indicated concentration of ADC in a total volume of 200 μL. Supernatant was harvested at 48 hours post addition of the THP1-Dual™ cells. To measure Lucia reporter signal, 10 μL of supernatant was combined with 40 μL of QUANTI-Luc™ Luminescence assay reagent (Invivogen PN: rep-qlc1) in a 96-well clear flat bottom tissue culture-treated black polystyrene plate (Corning Costar PN: 3603) and read on a Perkin Elmer Envision plate reader. In some experiments, HEK 293T cells engineered to express a murine protein typically expressed by immune cells (target antigen C— an immune cell antigen) were plated as above instead of Renca tumor cells. Cancer Cell Direct Cytotoxicity Assay

Cancer cells were counted and plated in 40 μL complete growth media in 384-well, white-walled tissue culture treated plates (Corning). Cell plates were incubated at 37° C. and with 5% CO₂ overnight to allow the cells to equilibrate. Stock solutions containing ADCs or free drugs were serially diluted in RPMI-1640+20% fetal bovine serum (FBS). 10 μL of each concentration were then added to each cell plate in duplicate. Cells were then incubated at 37° C. and with 5% CO₂ for 96 hours, upon which, the cell plates were removed from the incubator and allowed to cool to room temperature for 30 minutes prior to analysis. CellTiter-Glo® luminescent assay reagent (Promega Corporation, Madison, WI) was prepared according to Promega's protocol. 10 μL of CellTiter-Glo® were added to assay plates using a Formulatrix Tempest liquid handler (Formulatrix) and the plates were protected from light for 30 minutes at room temperature. The luminescence of the samples was measured using an EnVision Multimode plate reader (Perkin Elmer, Waltham, MA). Raw data were analyzed in Graphpad Prism (San Diego, CA) using a nonlinear, 4-parameter curve fit model [Y=Bottom+(Top−BottomY (1+10\hat{ }((Log EC50−X)*HillSlope))]. Results are reported as X50 values, which are defined as the concentration of ADC or free drug required to reduce cell viability to 50%.

SU-DHL-1 Assay

Potency of ADCs was evaluated using the SU-DHL-1 lymphoma cells. Cells were cultured in RPMI-1640 (Gibco) with 10% heat-inactivated fetal bovine serum, Pen-Strep (100 U/mL-100 μg/mL, Gibco), HEPES (10 mM, Gibco)), sodium pyruvate (1 mM, Gibco), MEM non-essential amino acids (1×, Gibco), GlutaMAX (1×, Gibco), and beta-mercaptoethanol (55 μM, Gibco). Cells were plated in a 96-well flat bottom tissue culture-treated clear polystyrene plate (Corning Costar #3596) at ˜100,000 cells per well in 200 μL with the indicated concentration of ADC. After 48 hours, the 50 μL supernatant was harvested and cytokine production was evaluated using a MILLIPLEX MAP Human Cytokine/Chemokine Magnetic Bead panel (HCYTOMAG-60K custom 8-plex kit: IL-6, IL-8, MCP1, TNFα, GRO, IP-10, MIP1α, and MIP1β). Cell viability was evaluated by adding 100 μL CellTiter-Glo® luminescent assay reagent (Promega Corporation, Madison, WI) to remaining 150 μL of cells in the plate and transferring the mixture to a 96-well black-walled plate (Corning Costar #3603). Plates were protected from light for 30 minutes at room temperature, and the luminescence of the samples was measured using an EnVision Multimode plate reader (Perkin Elmer, Waltham, MA).

Results from In Vitro Biological Assays

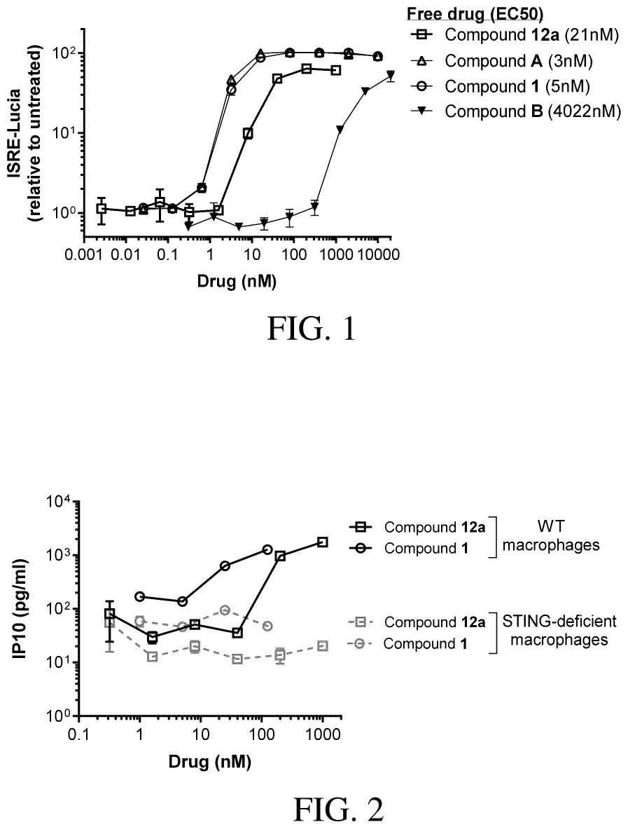

STING agonist compounds were assessed for their ability to activate THP1-Dual™ reporter cells, a human monocytic cell line in which type I interferon (IRF) signaling can be monitored via a secreted luciferase reporter protein (Lucia). THP1-Dual™ cells were treated with increasing concentrations of the agonists for 24h, then supernatants were harvested and the Lucia reporter signal was quantified using QUANTI-Luc™ Luminescence assay reagent. Compound A and compound 1 were significantly more potent than (2′, 3′)-Rp,Rpc-diAMPS disodium (Compound B) and activated the Lucia reporter with EC50 values of 3 and 5 nM respectively. Compound 12a was less potent than compound 1 and compound A (FIG. 1, EC₅₀ value of 21 nM). Both compound 1 and 12a induced cytokine production when used to stimulate wild type (WT), but not STING-deficient, murine bone marrow-derived macrophages, indicating the activity of these compounds is STING-dependent (FIG. 2).

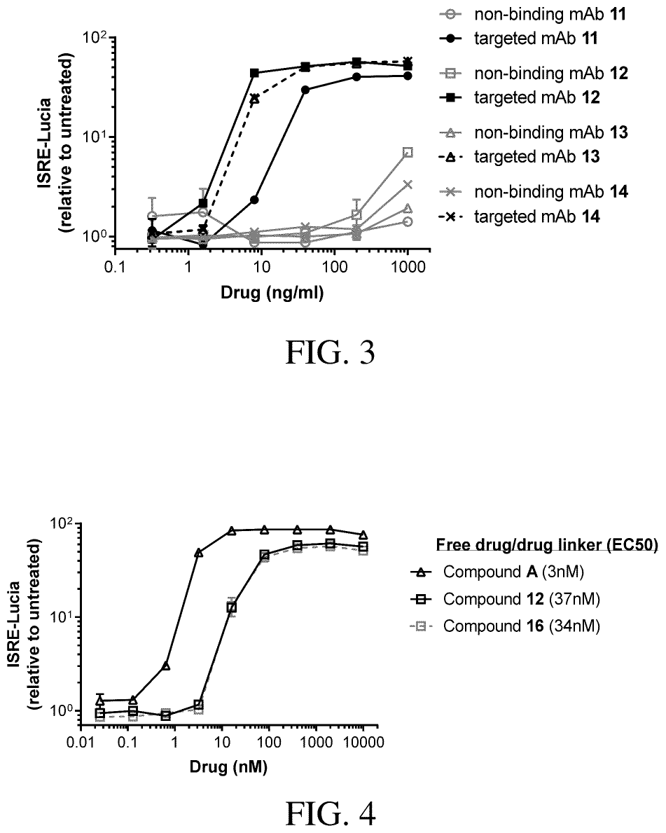

The STING agonist compounds were conjugated to both targeted and non-binding antibodies and the resulting ADCs were assessed for their ability to activate THP1-Dual™ reporter cells. Compound 1 was conjugated using a cleavable glucuronide-based linker (11). Compound 12a was conjugated using a non-cleavable, cleavable peptide-based, and cleavable glucuronide-based linker (Compounds 12, 14 and 13, respectively). THP1-Dual™ cells were treated with increasing concentrations of ADCs with a non-binding or targeted mAb conjugated to a compound for 48h, then supernatants were harvested, and the Lucia reporter signal was quantified using QUANTI-Luc™ Luminescence assay reagent. Although compound 12a was less potent than compound 1 as a free drug (FIG. 1), compound 12a was more potent when conjugated to a targeted mAb via a cleavable glucuronide linker (13) than the similar compound 1 conjugate (11). Furthermore, compound 12a was more potent when conjugated to a targeted mAb via a non-cleavable linker (12) than either cleavable linker 13 or 14 (FIG. 3), demonstrating that conjugation of STING agonist small molecules to an antibody can increase their potency.

Compound 12 and the cysteine adduct (compound 16) that is released upon cleavage of the mAb conjugate in the endo-lysosome were assessed for their ability to activate THP1-Dual™ reporter cells. THP1-Dual™ cells were treated with increasing concentrations of the compounds for 24h, then supernatants were harvested and the Lucia reporter signal was quantified using QUANTI-Luc™ Luminescence assay reagent. Both compound 12 and compound 16 were active with EC50 values (37 nM and 34 nM, respectively) similar to the parent free drug 12a (21 nM, FIG. 4 and FIG. 1).

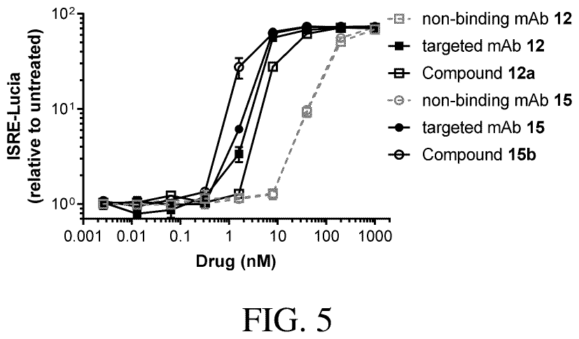

Compound 15b was also evaluated, both as a free drug and when conjugated to a targeted antibody using a non-cleavable linker (15). THP1-Dual™ cells were treated with increasing concentrations of free drug or ADCs with a non-binding or targeted mAb conjugated to a compound for 48h; then supernatants were harvested, and the Lucia reporter signal was quantified using QUANTI-Luc™ Luminescence assay reagent. Compound 15b was more potent than 12a, while the potency of the ADC of 15 was similar to that of the ADC of 12 when linked to the same targeted mAb (FIG. 5).

Compound 12a was conjugated to both targeted and non-binding antibodies using a variety of non-cleavable linkers (12, 17, 19-24) and the resulting ADCs were assessed for their ability to activate THP1-Dual™ reporter cells. All conjugates with the targeted mAb were active with EC50 values ranging from ˜1.7-7.3 ng/mL (Table 1). We also evaluated the ability of these linkers to directly kill cancer cells when conjugated to targeted mAbs binding tumor antigen A or antigen B (CD30). All conjugates were active in a subset of cancer cell lines (regardless of target antigen expression), indicating target-independent killing of some cancer cells; compounds 1, 12a, and 16 also demonstrated direct cytotoxic activity on a subset of cancer cell lines (Table 2; targeted mAb A conjugates comprise a mAb targeting the tumor antigen A conjugated to various drug linker compounds; targeted mAb B conjugates comprise the cAC10 mAb targeting CD30 conjugated to various drug linkers).

TABLE 1
Activity of target STING agonist ADCs in
THP1-Dual ™ reporter cells.

| Compound | EC₅₀ (ng/mL)** |
|---|---|
| 12 | 4.2 |
| 17 | 2.2 |
| 19 | 7.3 |
| 20 | 1.7 |
| 21 | 4.2 |
| 22 | 4.7 |
| 23 | 2.4 |
| 24 | 2.1 |
| 25 | 307 |
| 26 | 14.4 |
| 27 | 10.4 |
| 66 | >10,000 |
| 67 | >10,000 |
| 68 | >10,000 |
| 69 | >10,000 |
| 96 | >10,000 |
| 97 | * |
| 98 | >10,000 |
| 99 | * |
| 100 | * |
| 101 | >10,000 |
| 102 | >10,000 |
| 103 | >10,000 |
| 104 | >10,000 |
| 105 | 52.5 |
| 106 | >10,000 |
| 107 | >10,000 |
| 108 | 63.5 |
| 109 | >10,000 |
| 110 | >10,000 |
| 111 | 13.2 |
| 112 | 2 |
| 121 | 3.0 |
| 122 | 5.6 |
| 123 | 1.6 |
| 124 | 9.1 |
| 125 | 1.4 |
| 126 | >10,000 |
| 128 | >10,000 |
| 131 | 3.6 |
| 133 | 12.2 |
| 134 | 10.3 |
| 144 | >10,000 |
| 145 | >10,000 |
| 146 | >10,000 |
| 147 | >10,000 |
| 148 | >10,000 |
| 149 | >10,000 |
| 150 | 7.9 |
| 151 | >10,000 |
| 152 | 6.5 |

* ADCs with >10% aggregate were not evaluated
**For some compounds, EC50 values comprise the average value from multiple experiments

TABLE 2
Direct cytotoxicity data of various ADCs and compounds on human cancer cell lines.

| Target A expression | + | + | + | + | + | + | + | + | + | + | + |
|---|---|---|---|---|---|---|---|---|---|---|---|
| Target B expression | − | − | − | + | + | + | + | − | − | − | − |
| | ADC X50 (ng/mL) | | | | | | | | | | |
| | 786-O | A2058 | BxPC3 | DEL | DELBVR | Karpas299 | L540cy | Ls174T | MDAMB231 | MOLM-13 | SU-DHL-4 |
| Targeted mAb | >1K | >1K | >1K | 0.01 | 0.04 | 1 | 1 | >1K | >1K | 1 | >1K |
| A 12 (8 load) | | | | | | | | | | | |
| Targeted mAb | >1K | >1K | >1K | 0.012 | 0.03 | 3 | 2 | >1K | >1K | 12 | >1K |
| A 12 (4 load) | | | | | | | | | | | |
| Targeted mAb | >1K | >1K | >1K | <0.004 | 0.004 | 1 | 1 | >1K | >1K | 2 | >1K |
| B 12 (8 load) | | | | | | | | | | | |
| Targeted mAb | >1K | >1K | >1K | 0.01 | 0.1 | 2 | 1 | >1K | >1K | 7 | >1K |
| B 12 (4 load) | | | | | | | | | | | |
| Targeted mAb | >1K | >1K | >1K | <0.004 | 0.004 | 1 | 1 | >1K | >1K | 2 | >1K |
| A 17 (8 load) | | | | | | | | | | | |
| Targeted mAb | >1K | >1K | >1K | 0.1 | 0.03 | 5 | 2 | >1K | >1K | 26 | >1K |
| A 17 (4 load) | | | | | | | | | | | |
| Targeted mAb | >1K | >1K | >1K | <0.004 | <0.004 | 1 | 0.2 | >1K | >1K | 2 | >1K |
| B 17 (8 load) | | | | | | | | | | | |
| Targeted mAb | >1K | >1K | >1K | 0.004 | 0.01 | 1 | 1 | >1K | >1K | 25 | >1K |
| B 17 (4 load) | | | | | | | | | | | |
| Targeted mAb | >1K | >1K | >1K | 0.01 | 0.03 | 4 | 1 | >1K | >1K | 2 | >1K |
| A 21 (8 load) | | | | | | | | | | | |
| Targeted mAb | >1K | >1K | >1K | 0.1 | 0.2 | 7 | 2 | >1K | >1K | 20 | >1K |
| A 21 (4 load) | | | | | | | | | | | |
| Targeted mAb | >1K | >1K | >1K | <0.004 | 0.01 | 1 | 0.4 | >1K | >1K | 2 | >1K |
| B 21 (8 load) | | | | | | | | | | | |
| Targeted mAb | >1K | >1K | >1K | 0.02 | 0.1 | 2 | 1 | >1K | >1K | 2 | >1K |
| A 22 (6 load) | | | | | | | | | | | |
| Targeted mAb | >1K | >1K | >1K | 0.03 | 0.2 | 29 | 4 | >1K | >1K | 29 | >1K |
| A 22 (2 load) | | | | | | | | | | | |
| Targeted mAb | >1K | >1K | >1K | 0.01 | 0.1 | 1 | 1 | >1K | >1K | 3 | >1K |
| B 22 (6 load) | | | | | | | | | | | |
| Targeted mAb | >1K | >1K | >1K | <0.004 | 0.02 | 1 | 1 | >1K | >1K | 1 | >1K |
| A 19 (8 load) | | | | | | | | | | | |
| Targeted mAb | >1K | >1K | >1K | 0.01 | 0.1 | 3 | 5 | >1K | >1K | 13 | >1K |
| A 19 (4 load) | | | | | | | | | | | |
| Targeted mAb | >1K | >1K | >1K | <0.004 | 0.01 | 0.3 | 0.3 | >1K | >1K | 0.4 | >1K |
| B 19 (8 load) | | | | | | | | | | | |
| Targeted mAb | >1K | >1K | >1K | <0.004 | 0.02 | 0.5 | 1 | >1K | >1K | 1 | >1K |
| B 19 (4 load) | | | | | | | | | | | |
| Targeted mAb | >1K | >1K | >1K | 0.01 | 0.02 | 0.2 | 0.3 | >1K | >1K | 1 | >1K |
| A 20 (8 load) | | | | | | | | | | | |
| Targeted mAb | >1K | >1K | >1K | 0.01 | 0.02 | 1 | 1 | >1K | >1K | 10 | >1K |
| A 20 (4 load) | | | | | | | | | | | |
| Targeted mAb | >1K | >1K | >1K | <0.004 | <0.004 | 0.1 | 0.1 | >1K | >1K | 0.2 | >1K |
| B 20 (8 load) | | | | | | | | | | | |
| Targeted mAb | >1K | >1K | >1K | <0.004 | 0.02 | 0.1 | 0.5 | >1K | >1K | 1 | >1K |
| B 20 (4 load) | | | | | | | | | | | |
| Targeted mAb | >1K | >1K | >1K | <0.004 | 0.01 | 0.3 | 1 | >1K | >1K | 1 | >1K |
| A 24 (8 load) | | | | | | | | | | | |
| Targeted mAb | >1K | >1K | >1K | 0.01 | 0.01 | 1 | 4 | >1K | >1K | 11 | >1K |
| A 24 (4 load) | | | | | | | | | | | |
| Targeted mAb | >1K | >1K | >1K | <0.004 | 0.01 | 0.03 | 0.5 | >1K | >1K | 1 | >1K |
| B 24 (8 load) | | | | | | | | | | | |
| Targeted mAb | >1K | >1K | >1K | <0.004 | 0.01 | 0.2 | 2 | >1K | >1K | 4 | >1K |
| B 24 (4 load) | | | | | | | | | | | |
| Targeted mAb | >1K | >1K | >1K | 0.01 | 0.03 | 1 | 1 | >1K | >1K | 1 | >1K |
| A 23 (8 load) | | | | | | | | | | | |
| Targeted mAb | >1K | >1K | >1K | 0.02 | 0.05 | 4 | 5 | >1K | >1K | 8 | >1K |
| A 23 (4 load) | | | | | | | | | | | |
| Targeted mAb | >1K | >1K | >1K | <0.004 | 0.01 | 0.1 | 0.2 | >1K | >1K | 0.3 | >1K |
| B 23 (8 load) | | | | | | | | | | | |
| Targeted mAb | >1K | >1K | >1K | 0.002 | 0.02 | 0.3 | 0.3 | >1K | >1K | 2 | >1K |
| B 23 (4 load) | | | | | | | | | | | |
| | Compound X50 (nM)) | | | | | | | | | | |
| Compound 1 | >1K | >1K | >1K | 2 | 16 | >1K | 31 | >1K | >1K | 42 | >1K |
| Compoud 12a | >1K | >1K | >1K | 3 | 111 | >1K | 83 | >1K | >1K | 87 | >1K |
| Compound 16 | >1K | >1K | >1K | 4 | 7 | >1K | 29 | >1K | >1K | 27 | >1K |

Multiple additional compounds were synthesized and evaluated for their ability activate THP1-Dual™ reporter cells. Several compounds were active with EC50 values ranging from 1.3 nM (compound 27e) to 6337 nM (compound 126a, Table 3). Compounds with minimal activity up to 10 μM are listed in Table 3 as having an EC5 value of >10,000 nM. Several compounds were conjugated to targeted (Table 1) and non-binding antibodies (not shown) via cleavable or non-cleavable drug linkers and the resulting ADCs were assessed for their ability to activate THP1-Dual™ reporter cells. Conjugates with drug linkers 25-27, 105, 108, 111-112 and 121-125 were active with EC50 values ranging from 1.4 to 307 ng/mL (Table 1). All other conjugates tested were not active up to 10 μg/mL in this assay, including conjugates with drug linkers derived from active small molecules (Table 3, Table 1) thus highlighting the challenges of developing active ADCs targeting the STING pathway.

TABLE 3
Activity of STING agonist small molecules in
THP1-Dual ™ reporter cells.

| Compound | EC₅₀ (nM)** |
|---|---|
| A | 3 |
| 1 | 5 |
| 12 | 37 |
| 12a | 21 |
| 15b | 5.8 |
| 16 | 52/34 |
| 17 | 66.28 |
| 19 | 190.2 |
| 20 | 53.91 |
| 21 | 2632 |
| 23 | 62.55 |
| 24 | 83.29 |
| 25f | 12 |
| 25g | 1576 |
| 26e | 5 |
| 26f | 69 |
| 27e | 1.3 |
| 27f | 38 |
| 28 | 954 |
| 29 | 52 |
| 30 | 87 |
| 31 | 175.6 |
| 32 | 44.5 |
| 33 | >10,000 |
| 35 | >10,000 |
| 37 | >10,000 |
| 38 | >10,000 |
| 39 | >10,000 |
| 40 | >10,000 |
| 41 | >10,000 |
| 42 | >10,000 |
| 43 | >10,000 |
| 44 | >10,000 |
| 45 | >10,000 |
| 46 | >10,000 |
| 47 | >10,000 |
| 48 | >10,000 |
| 49 | >10,000 |
| 50 | >10,000 |
| 51 | >10,000 |
| 52 | >10,000 |
| 53 | >10,000 |
| 54 | >10,000 |
| 55 | >10,000 |
| 56 | >10,000 |
| 57 | >10,000 |
| 58 | >10,000 |
| 59 | >10,000 |
| 60 | >10,000 |
| 62 | >10,000 |
| 63 | 2508 |
| 64/65 | 388.3 |
| 66b | 1159 |
| 68a | 2238 |
| 70 | 1.6 |
| 71 | 70.11 |
| 72 | 275.3 |
| 73 | 216.25 |
| 74 | 35.98 |
| 75 | 57.73 |
| 76 | 58.8 |
| 79 | >10,000 |
| 80 | >10,000 |
| 81 | >10,000 |
| 82 | >10,000 |
| 83 | >10,000 |
| 84 | >10,000 |
| 85 | >10,000 |
| 86 | 2465 |
| 87 | >10,000 |
| 88 | 394 |
| 89 | 1324 |
| 90 | >10,000 |
| 91 | 350 |
| 92 | 1190 |
| 93 | >10,000 |
| 94 | 132.8 |
| 95 | 12.9 |
| 114 | 9.4 |
| 115 | 17.4 |
| 116 | 11.6 |
| 117 | 14.6 |
| 118 | 23.6 |
| 119 | 49.9 |
| 120 | 5.2 |
| 126a | 6337 |
| 126b | >10,000 |
| 127 | 301 |
| 128a | >10,000 |
| 129 | 836 |
| 130 | 2260 |
| 132 | 37.4 |
| 135 | 2011 |
| 136 | >5,000 |
| 137 | 1685 |
| 138 | >5,000 |
| 139 | >4,000 |
| 140 | 1793 |
| 141 | 159 |
| 142 | 1123 |
| 143 | 155 |
| 154 | >10,000 |
| 155 | 94 |
| 156 | 1561 |
| 157 | 51 |

**For some compounds, EC50 values comprise the average value from multiple experiments

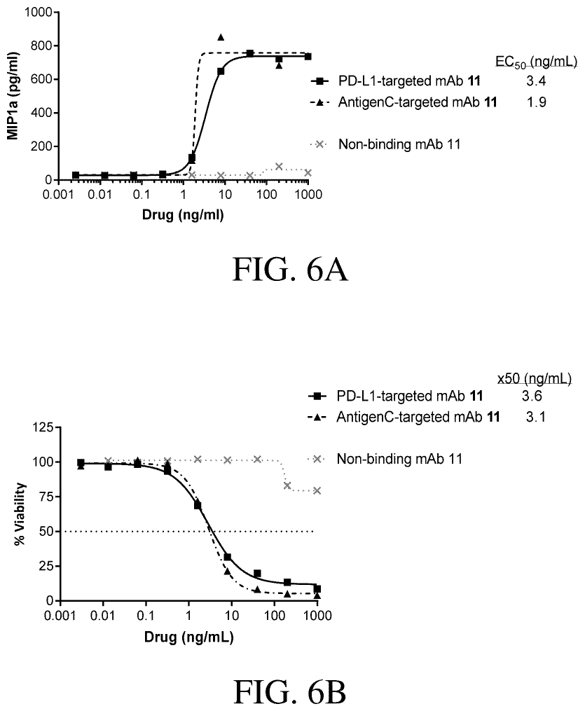

Compound 1 was conjugated to a non-binding antibody as well as antigen C and PD-L1-targeted mAbs using the cleavable linker 11 and the resulting ADCs were assessed for their ability to induce cytokine production and direct cytotoxicity by SU-DHL-1 cells. Conjugates targeting antigen C and PD-L1, but not the non-binding conjugate, induced robust production of the cytokine MIP-1α and led to SU-DHL-1 cell death (FIG. 6A and FIG. 6B).

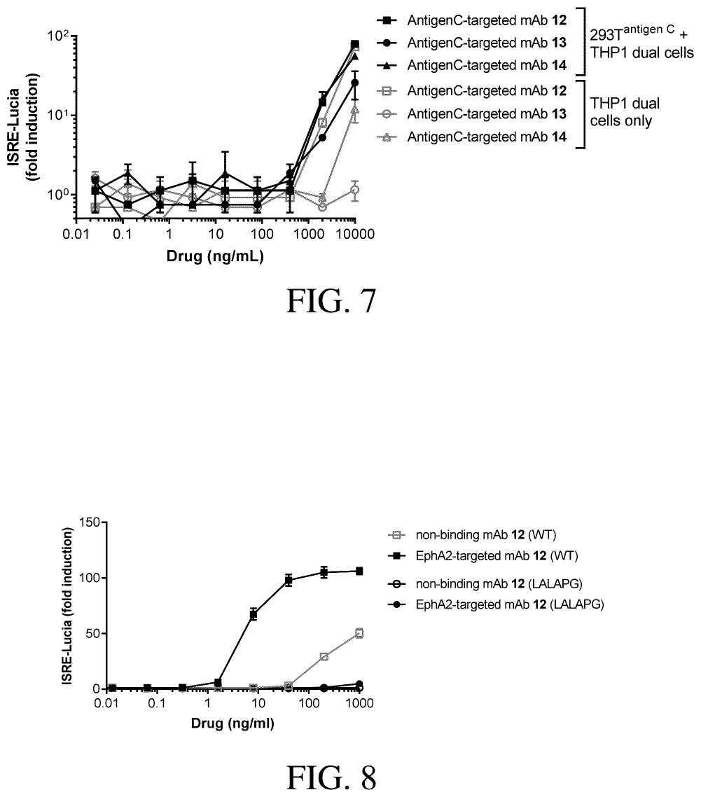

The ability of conjugates to activate THP1 dual reporter immune cells in a bystander manner was evaluated. Conjugates consisting of an antibody targeting antigen C with a hIgG1 LALAPG Fc backbone conjugated to compound 12, 13, and 14 demonstrated some bystander activity when THP1 dual cells were co-cultured with HEK 293T cells engineered to express antigen C (FIG. 7). Conjugates consisting of the h1C1 antibody targeting EphA2 with a mIgG2a WT or LALAPG Fc backbone (see, e.g., Schlothauer et al., Protein Engineering, Design and Selection, 2016, 29(10):457-466; and Hezareh et al., Journal of Virology, 2001, 75(24):12161-12168, each of which is incorporated herein by reference in its entirety) conjugated to compound 12 also demonstrated bystander activity when THP1 dual cells were co-cultured with murine Renca tumor cells. Markedly enhanced bystander activity was observed with conjugates with an intact WT Fc backbone (FIG. 8).

### Example 3

### In Vivo Evaluation of Anti-Tumor Immune Responses Induced by Sting Agonist ADCS

### Experimental Procedures for In Vivo Studies

In vivo Cytokine Assay

Cytokines were measured in mouse plasma harvested at 3, 6, 24, or 48 hours after treatment with compounds or ADCs using a Milliplex MAP mouse cytokine/chemokine magnetic bead panel assay kit (MCYTOMAG-70k custom 11-plex kit: MCP1, MIP1α, MIP1β, TNFα, IFNγ, IL-10, IL-12p70, IL-6, IL-1β, IP10, RANTES) and analyzed using a Luminex™ MAGPIX™ Instrument System. Values that were outside of the standard curve range (<3.2 or >10,000 μg/mL) were excluded from calculation of the mean values.

### In Vivo Anti-Tumor Activity Studies

Renca Cancer Cells

Renca cancer cells (ATCC) were cultured in RPMI-1640 (ATCC) with 10% heat-inactivated fetal bovine serum, Pen-Strep (100 U/mL-100 μg/mL), MEM non-essential amino acids (lx), sodium pyruvate (1 mM), and L-glutamine (2 mM). Renca cancer cells were implanted (2*10⁶ cells in 200 μL 25% Matrigel) subcutaneously into Balb/c female mice. In some experiments, Renca tumor cells were engineered to express the indicated murine or human target antigen.

When tumor volumes reached 100 mm³, the mice were dosed with either compounds or ADCs by intraperitoneal or intravenous injection at the indicated dosing schedule and tumor volumes were monitored twice weekly. Compounds were formulated in 40% PEG400 in saline.

CT26 Cancer Cells

CT26 cancer cells (ATCC) were cultured in RPMI 1640 modified with 1 mM Sodium Pyruvate, 10 mM HEPES, 2.8 mL 45% Glucose (1.25 g) and supplemented with 10% fetal bovine serum and 1% Pen/Strep/Glutamine. CT26 cancer cells were implanted (0.5*106 cells in 200 uL serum-free RPMI 1640) subcutaneously into Balb/c mice.

MC38 Cancer Cells

MC38 cancer cells (Kerafast) were cultured in DMEM with 10% heat-inactivated fetal bovine serum, Pen-Strep (100 U/mL-100 μg/mL), MEM non-essential amino acids (1×), sodium pyruvate (1 mM), and L-glutamine (2 mM). MC38 cancer cells were implanted (1*10⁶ cells in 100 uL 25% Matrigel) subcutaneously into C57B/6 mice.

In some experiments, tumor-bearing mice that achieved complete tumor regression following ADC treatment were “rechallenged” with MC38 tumor cells; MC38 cancer cells were implanted (1*10⁶ cells in 100 uL 25% Matrigel) subcutaneously into the opposite flank of C57BL/6 mice.

4T1 Cancer Cells

4T1 cancer cells (ATCC) were cultured in RPMI with 10% heat-inactivated fetal bovine serum and implanted (0.02*10⁶ cells in 200 uL plain RPMI) subcutaneously into Balb/c mice.

Results from In Vivo Studies

Renca Cancer Cells

A syngeneic system was used to assess the ability of the STING agonist ADCs to induce immune responses in vivo and drive an anti-tumor immune response. The Renca system is a subcutaneous, mouse renal adenocarcinoma model. Female Balb/c mice were implanted with 2×10⁶ Renca cells subcutaneously in the flank on day 0. When mean tumor size of 100 mm³ (measured by using the formula Volume (mm³)=0.5*Length*Width² where length is the longer dimension) was reached mice were randomized into treatment groups of ≥5 mice per group. Animals were then treated intraperitoneally (ADCs or compounds) or intravenously (compounds) with the indicated treatment every 7 days, for 3 doses total (or as indicated). Tumor length and width and the weight of the animals was measured throughout the study and tumor volume was calculated using the formula above. Animals were followed until the tumor volume reached ˜1000 mm³; animals were then euthanized.

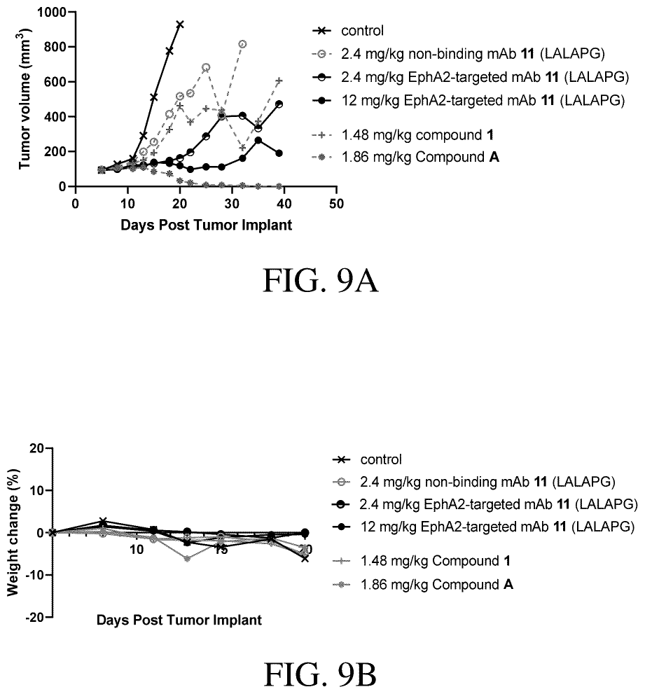

The anti-tumor activity of compound 1 compared to the cleavable linker 11 conjugated to a non-binding or EphA2-targeted mAb (mIgG2a LALAPG backbone; see, e.g., Schlothauer et al., Protein Engineering, Design and Selection, 2016, 29(10):457-466; and Hezareh et al., Journal of Virology, 2001, 75(24):12161-12168, each of which is incorporated herein by reference in its entirety) was evaluated; note that all EphA2-targeted mAb conjugates described herein consist of the h1C1 mIgG2a mAb conjugated to various drug linker compounds. When animals were treated with the Compound 1 or the non-binding mAb conjugate of 11, some tumor growth delay was observed; however, tumor growth delay was significantly enhanced with the EphA2-targeted mAb conjugate of 11, especially at the higher 12 mg/kg dose (FIG. 9A), clearly demonstrating the anti-tumor benefit of delivering STING agonists using a targeted ADC.

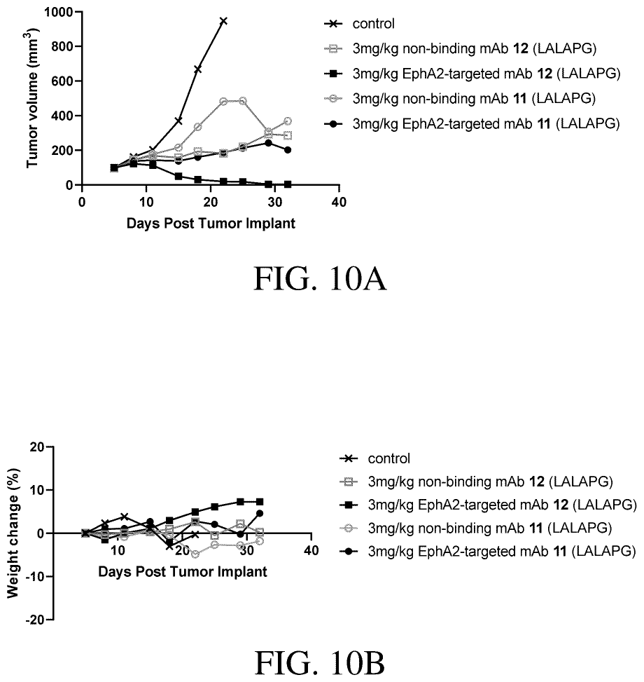

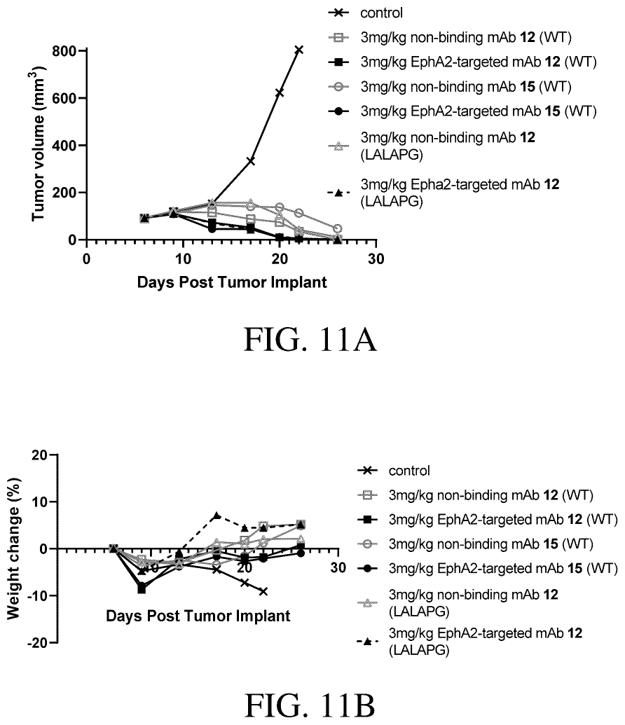

In the next in vivo study, the anti-tumor activity of the non-cleavable linker compound 12 conjugated to a non-binding or EphA2-targeted mAb (mIgG2a LALAPG backbone) was evaluated. EphA2-targeted mAb conjugate of 12 exhibited robust anti-tumor activity and was surprisingly more active than the ADC of 11 conjugated to the same EphA2-targeted mAb (FIG. 10A). In the next in vivo study, the anti-tumor activity of the non-cleavable linker 15 conjugated to a non-binding or EphA2-targeted mAb (mIgG2a WT backbone) was evaluated. EphA2-targeted mAb conjugates of 15 exhibited robust anti-tumor activity that was similar to the corresponding ADC of 12 (FIG. 11A). In this study, the activity of 12 conjugated to an EphA2-targeted antibody with a mIgG2a WT and LALAPG backbone was also evaluated, and both conjugates were similarly active. This was a surprising finding, given that the in vitro bystander assay indicates that an intact WT Fc backbone significantly enhances bystander immune cell activation compared to LALAPG Fc backbone (FIG. 8).

Compound 1 and all antibody conjugates of 11 and 12 on a mIgG2a LALAPG backbone were well tolerated—average weight loss was <˜5% after the 1^{st} and 2^{nd} dose of the treatment. The STING agonist compound A was less well tolerated—with mice exhibiting on average 6.2% weight loss after the 2^{nd} dose (FIGS. 9B, 10B, and 11B). Moreover, EphA2-targeted mAb conjugates of 12 and 15 with a mIgG2a WT backbone at the 3 mg/kg dose level were less well tolerated than the conjugate of 12 with a LALAPG backbone—with mice treated with targeted WT backbone ADCs exhibiting ˜8% weight loss (FIG. 11B).

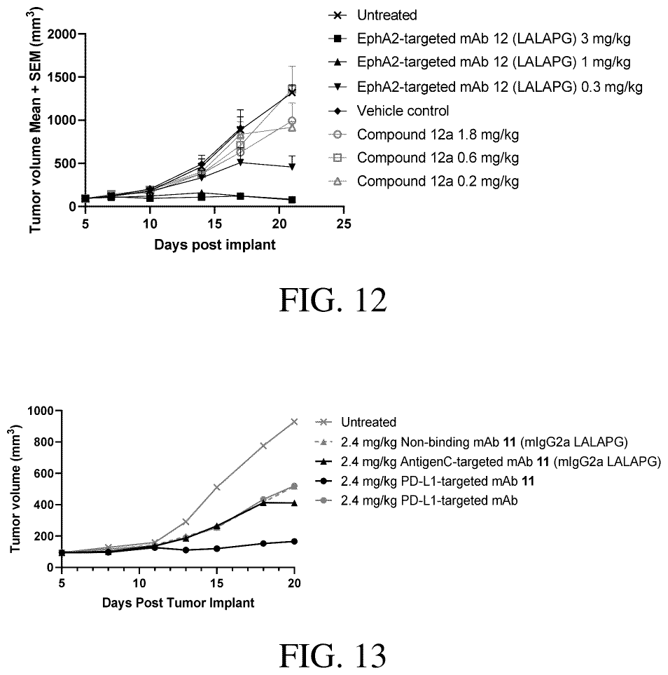

In the next in vivo study, the anti-tumor activity of the non-cleavable linker compound 12 conjugated to an EphA2-targeted mAb (mIgG2a LALAPG backbone) as well as unconjugated Compound 12a was evaluated. The EphA2-targeted mAb conjugates of 12 exhibited robust anti-tumor activity at doses of 1 mg/kg and 3 mg/kg, while Compound 12a had limited anti-tumor efficacy (FIG. 12). Collectively, this suggests that STING agonist compounds (e.g., compounds 1 and 12a) that are inactive in vivo in tumor models can be converted into active therapeutics by conjugation to an antibody (e.g., targeted mAb conjugates of 11 and 12).

Systemic cytokine production in response to the free drugs and conjugates was measured as a proxy for systemic activity. Compound 1 and all antibody conjugates of 11, 12 and 15 induced very little pro-inflammatory cytokine (IL-6 and TNF) production. On the other hand, compound A and compound 12a induced robust production of IL-6 and TNF (Table 4, Table 5, and Table 6). Moreover, EphA2-targeted conjugates of 11 and 12 with a WT Fc backbone induced more systemic MIP1α, MIP1β, and MCP-1 expression than the conjugate of 12 with a LALAPG Fc backbone. This indicates that, in the Renca tumor model with the specific EphA2-targeted antibodies described in FIGS. 10-12 dosed at 3 mg/kg q7dx3, a LALAPG Fc backbone may reduce on-target toxicity (systemic cytokines/weight loss), without impacting anti-tumor efficacy. This also indicates that conjugation of a STING agonist compound (e.g., compound 12a vs. the targeted mAb conjugate of 12) may improve both efficacy and safety (reduce systemic cytokines).

TABLE 4
Cytokine production in peripheral blood (plasma) in Renca tumor-bearing mice upon treatment with various ADCs comprising a non-
binding or EphA2-targeted mAb with a mIgG2a LALAPG backbone conjugated to compound 11 or 12, or compound 1 or Compound A.

| | | | 3 mg/kg | 3 mg/kg | | | | 2.4 mg/kg | | | | |
|---|---|---|---|---|---|---|---|---|---|---|---|---|
| | | | non- | non- | 3 mg/kg | 3 mg/kg | | non- | 2.4 mg/g | 12 mg/kg | 1.48 mg/kg | 1.86 mg/kg |
| | Time | | binding | binding | targeted | targeted | | binding | targeted | targeted | Compound | Compound |
| | (h) | control | mAb 11 | mAb 12 | mAb 11 | mAb 12 | control | mAb 11 | mAb 11 | mAb 11 | 1 | A |
| IFNγ | 3 | <3.2 | 1.1 | 0.4 | <3.2 | <3.2 | <3.2 | <3.2 | <3.2 | <3.2 | <3.2 | 0.3 |
| | 6 | <3.2 | 15.6 | 7.1 | 8.5 | 22.6 | <3.2 | <3.2 | 1.3 | 4.3 | 0.2 | 71.8 |
| | 24 | <3.2 | <3.2 | 1.4 | <3.2 | 3.0 | <3.2 | <3.2 | <3.2 | 0.9 | <3.2 | <3.2 |
| IL1β | 3 | 3.2 | 5.3 | 4.3 | 4.3 | 5.3 | 0.4 | 0.3 | 0.5 | 0.3 | 0.7 | 1.5 |
| | 6 | 3.2 | 4.3 | 4.3 | 5.3 | 6.4 | 0.3 | 1.7 | 0.3 | 1.8 | 1.5 | 0.6 |
| | 24 | 3.2 | 4.3 | 4.8 | 4.3 | 5.8 | 0.3 | 0.3 | 0.3 | 0.3 | 0.8 | 0.3 |
| IL-6 | 3 | 9.2 | 120.1 | 23.8 | 150.5 | 109.5 | 154.7 | 14.3 | 5.5 | 37.7 | 95.7 | 3553.0 |
| | 6 | 26.9 | 472.1 | 176.6 | 305.6 | 404.4 | 0.7 | 162.2 | 81.9 | 303.4 | 6.8 | 731.9 |
| | 24 | 33.2 | 12.0 | 58.6 | 28.0 | 75.3 | 7.7 | 19.6 | 4.8 | 27.0 | 13.5 | 16.4 |
| IL-10 | 3 | <3.2 | <3.2 | <3.2 | <3.2 | <3.2 | <3.2 | <3.2 | <3.2 | 1.5 | 48.8 | 0.5 |
| | 6 | <3.2 | 3.9 | 4.3 | 4.8 | 6.4 | <3.2 | <3.2 | <3.2 | <3.2 | 0.8 | <3.2 |
| | 24 | <3.2 | <3.2 | <3.2 | <3.2 | <3.2 | <3.2 | <3.2 | <3.2 | 0.8 | 34.6 | <3.2 |
| IL12p70 | 3 | <3.2 | 5.9 | <3.2 | <3.2 | <3.2 | <3.2 | <3.2 | <3.2 | 17.6 | 174.5 | 0.2 |
| | 6 | <3.2 | <3.2 | <3.2 | <3.2 | 3.1 | <3.2 | <3.2 | <3.2 | <3.2 | <3.2 | 0.2 |
| | 24 | <3.2 | <3.2 | <3.2 | <3.2 | <3.2 | <3.2 | <3.2 | <3.2 | 23.4 | 140.0 | <3.2 |
| IP10 | 3 | 235.3 | 973.2 | 402.4 | 1069.7 | 1094.1 | 59.8 | 176.7 | 80.5 | 295.4 | 702.8 | 4785.0 |
| | 6 | 179.7 | 2976.0 | 2783.5 | 2963.1 | 3037.2 | 37.0 | 3977.4 | 4205.6 | 7374.2 | 3391.5 | 4976.1 |
| | 24 | 192.3 | 1129.0 | 2329.0 | 1019.3 | 2564.0 | 71.5 | 559.7 | 358.1 | 1524.0 | 551.1 | 889.6 |
| MCP-1 | 3 | 50.2 | 138.6 | 97.6 | 152.5 | 166.5 | 67.8 | 21.6 | 10.0 | 65.2 | 167.8 | 14494.4 |
| | 6 | 35.8 | 1168.1 | 615.0 | 1727.0 | 7542.0 | 19.7 | 3822.7 | 1811.8 | 10211.9 | 795.6 | 10776.8 |
| | 24 | 50.2 | 157.0 | 2374.8 | 284.9 | 7777.1 | 26.9 | 238.0 | 161.9 | 1486.0 | 332.6 | 539.5 |
| MIP1α | 3 | <3.2 | 26.1 | <3.2 | 26.1 | <3.2 | 24.1 | 3.0 | 13.9 | 10.0 | 55.6 | 1300.3 |
| | 6 | <3.2 | 36.4 | 44.5 | 38.4 | 67.1 | 10.0 | 49.1 | 24.0 | 108.5 | 38.9 | 208.8 |
| | 24 | <3.2 | <3.2 | 62.9 | 62.9 | 36.4 | 33.9 | 10.0 | 49.4 | 29.9 | 69.9 | 41.7 |
| MIP1β | 3 | <3.2 | 78.2 | 40.4 | 66.6 | 53.3 | 6.4 | 11.3 | 6.4 | 45.4 | 362.3 | 6365.1 |
| | 6 | <3.2 | 736.5 | 697.0 | 657.3 | 1267.3 | <3.2 | 469.0 | 197.9 | 1170.5 | 168.7 | 2153.8 |
| | 24 | <3.2 | 61.8 | 439.4 | 35.9 | 705.5 | <3.2 | 35.6 | 26.4 | 270.7 | 70.7 | 35.5 |
| RANTES | 3 | 4.4 | 6.4 | 1.3 | 7.8 | 5.3 | 2.9 | 2.2 | 1.7 | 3.4 | 10.6 | 91.6 |
| | 6 | <3.2 | 14.9 | 3.4 | 13.2 | 34.1 | 2.7 | 19.9 | 11.9 | 57.3 | 18.4 | 736.9 |
| | 24 | 4.3 | 25.5 | 186.5 | 22.3 | 239.1 | 3.5 | 18.2 | 11.9 | 90.6 | 15.6 | 113.2 |
| TNFa | 3 | <3.2 | 3.2 | <3.2 | <3.2 | <3.2 | 1.6 | 1.4 | 1.3 | 2.5 | 3.6 | 57.0 |
| | 6 | <3.2 | 12.9 | 7.2 | 10.2 | 4.6 | 0.8 | 3.8 | 2.1 | 6.2 | 1.9 | 17.3 |
| | 24 | <3.2 | <3.2 | 3.2 | <3.2 | 8.2 | 1.3 | 1.4 | 1.3 | 3.1 | 2.0 | 1.3 |

TABLE 5
Cytokine production in peripheral blood (plasma) in engineered Renca tumor-bearing mice upon treatment with various ADCs comprising
a non-binding or EphA2-targeted mAb with either a mIgG2a wild type (WT) or a mIgG2a LALAPG backbone conjugated to compounds 12 or 15.

| | | | 3 mg/kg non- | 3 mg/kg non- | | | 3 mg/kg non- | |
|---|---|---|---|---|---|---|---|---|
| | | | targeted mAb 12 | targeted mAb 12 | 3 mg/kg targeted | 3 mg/kg targeted | targeted mAb 15 | 3 mg/kg targeted |
| | Time (h) | control | (LALAPG) | (WT) | mAb 12 (LALAPG) | mAb 12 (WT) | (WT) | mAb 15 (WT) |
| IFNγ | 3 | 1.1 | 1.2 | 2.2 | 0.7 | 1.2 | 1.3 | 1.8 |
| | 6 | 0.8 | 8.9 | 5.2 | 12.1 | 19.3 | 8.5 | 17.5 |
| | 24 | 1.3 | 7.1 | 6.1 | 18.8 | 27.0 | 6.6 | 21.1 |
| | 48 | 2.0 | 1.5 | 2.0 | 2.1 | 2.0 | 1.8 | 2.0 |
| IL1β | 3 | 8.8 | 12.7 | 26.0 | 19.8 | 7.8 | 15.9 | 30.3 |
| | 6 | 8.8 | 35.5 | 14.7 | <3.2 | 27.0 | 27.3 | 19.0 |
| | 24 | 10.7 | 12.2 | 22.7 | 4.9 | 10.7 | 10.8 | 7.8 |
| | 48 | <3.2 | 4.9 | 22.7 | 7.8 | 2.1 | 13.7 | 10.7 |
| IL-6 | 3 | 5.1 | 12.7 | 12.6 | 71.6 | 61.6 | 12.0 | 105.8 |
| | 6 | 5.5 | 55.3 | 47.3 | 327.4 | 279.5 | 82.4 | 475.4 |
| | 24 | 3.5 | 33.4 | 25.2 | 61.1 | 80.8 | 41.3 | 63.0 |
| | 48 | 2.3 | 5.0 | 5.8 | 29.9 | 15.6 | 11.5 | 17.9 |
| IL-10 | 3 | 2.6 | 4.0 | 4.4 | 4.1 | 2.2 | 5.9 | 5.1 |
| | 6 | 2.5 | 8.2 | 4.8 | 5.2 | 9.6 | 8.4 | 6.3 |
| | 24 | 1.6 | 3.2 | 2.7 | 4.9 | 5.5 | 4.9 | 7.3 |
| | 48 | 0.6 | 0.6 | 2.7 | 1.7 | 1.0 | 2.6 | 2.1 |
| IL12p70 | 3 | 12.1 | 5.0 | 31.9 | 26.6 | <3.2 | 23.3 | 12.5 |
| | 6 | 0.9 | 48.9 | 13.7 | <3.2 | 23.5 | 29.0 | 10.3 |
| | 24 | 0.9 | 10.5 | 7.1 | 4.0 | 0.9 | 10.5 | 4.0 |
| | 48 | <3.2 | 0.9 | 20.1 | <3.2 | 0.9 | 7.1 | 0.9 |
| IP10 | 3 | 99.4 | 134.0 | 104.9 | 335.5 | 371.9 | 185.4 | 637.3 |
| | 6 | 71.0 | 2469.9 | 2651.6 | 2757.9 | 3219.0 | 2885.3 | 3422.1 |
| | 24 | 86.3 | 1798.9 | 1652.7 | 2219.9 | 2004.1 | 2616.0 | 2324.1 |
| | 48 | 127.6 | 738.5 | 822.9 | 824.4 | 819.2 | 1052.2 | 935.8 |
| MCP-1 | 3 | 63.2 | 120.4 | 165.9 | 135.5 | 96.9 | 142.7 | 276.1 |
| | 6 | 72.1 | 1368.6 | 1195.0 | 915.2 | 4492.9 | 1764.0 | 6594.8 |
| | 24 | 57.0 | 2083.8 | 1878.0 | 6406.5 | 8018.1 | 9546.4 | 15026.1 |
| | 48 | 42.1 | 285.6 | 599.9 | 794.1 | 934.2 | 1629.3 | 2234.6 |
| MIP1α | 3 | 137.3 | 142.9 | 205.0 | 166.4 | 101.3 | 187.3 | 163.7 |
| | 6 | 101.3 | 178.8 | 130.0 | 61.4 | 284.3 | 133.8 | 271.9 |
| | 24 | 133.8 | 120.7 | 153.3 | 101.3 | 227.0 | 193.7 | 205.2 |
| | 48 | <3.2 | 101.3 | 140.2 | 101.3 | 101.3 | 140.2 | 166.4 |
| MIP1β | 3 | <3.2 | <3.2 | 78.1 | <3.2 | 349.4 | 49.8 | 942.0 |
| | 6 | <3.2 | 578.4 | 850.5 | 735.3 | 2898.1 | 677.8 | 4342.5 |
| | 24 | <3.2 | 425.4 | 456.2 | 661.8 | 1493.2 | 1561.0 | 1387.7 |
| | 48 | <3.2 | 178.9 | 227.9 | 221.2 | 309.4 | 567.1 | 378.3 |
| RANTES | 3 | 5.6 | 8.0 | 11.0 | 9.1 | 6.7 | 10.1 | 11.0 |
| | 6 | 6.6 | 24.5 | 18.9 | 14.4 | 168.0 | 37.5 | 206.6 |
| | 24 | 5.6 | 77.1 | 52.5 | 338.9 | 174.8 | 335.7 | 772.2 |
| | 48 | 6.6 | 21.3 | 43.9 | 69.6 | 59.2 | 140.4 | 190.4 |
| TNFα | 3 | 4.6 | 6.2 | 6.8 | 7.7 | 4.3 | 11.0 | 9.8 |
| | 6 | 2.7 | 15.0 | 5.8 | 3.5 | 16.5 | 9.9 | 22.9 |
| | 24 | 5.4 | 7.7 | 7.7 | 6.6 | 8.8 | 13.3 | 11.0 |
| | 48 | 1.9 | 5.4 | 6.5 | 5.4 | 5.4 | 6.6 | 7.6 |

TABLE 6
Cytokine production in peripheral blood (plasma) in Renca tumor-bearing mice upon treatment with ADCs
comprising an EphA2-targeted mAb with mIgG2a LALAPG backbone conjugated to compound 12 or compound 12a.

| | | | | | | | | 0.3 mg/kg | 1.0 mg/kg | 3.0 mg/kg |
|---|---|---|---|---|---|---|---|---|---|---|
| | | | | | 0.2 mg/kg | 0.6 mg/kg | 1.8 mg/kg | targeted | targeted | targeted |
| | | | | | Compound | Compound | Compound | mAb 12 | mAb 12 | mAb 12 |
| | Dose # | Time (h) | Untreated | Vehicle | 12a | 12a | 12a | (LALAPG) | (LALAPG) | (LALAPG) |
| IFNg | 1 | 3 | <3.2 | <3.2 | <3.2 | <3.2 | <3.2 | <3.2 | <3.2 | <3.2 |
| | 1 | 6 | <3.2 | <3.2 | 4.6 | 18.3 | 36.4 | <3.2 | <3.2 | 9.4 |
| | 1 | 24 | <3.2 | <3.2 | <3.2 | <3.2 | <3.2 | <3.2 | 5.8 | 5.3 |
| | 1 | 48 | <3.2 | <3.2 | <3.2 | <3.2 | <3.2 | <3.2 | <3.2 | <3.2 |
| | 2 | 6 | <3.2 | <3.2 | 16.5 | 41.5 | 80.4 | 0.0 | 1.7 | 20.1 |
| IL-1b | 1 | 3 | 0.7 | 0.7 | 0.7 | 0.7 | 7.5 | 3.2 | 0.7 | 0.7 |
| | 1 | 6 | 0.7 | <3.2 | 0.7 | 5.7 | 0.7 | 3.2 | 11.0 | 5.7 |
| | 1 | 24 | 7.0 | 3.2 | 3.2 | 1.6 | 3.2 | 2.4 | 0.7 | 3.2 |
| | 1 | 48 | 3.2 | 5.7 | 0.7 | 5.7 | 3.2 | 3.2 | 3.2 | 5.8 |
| | 2 | 6 | <3.2 | <3.2 | 2.7 | 5.8 | 4.6 | 2.7 | 2.7 | <3.2 |
| IL-6 | 1 | 3 | 3.3 | 16.6 | 780.0 | 1899.7 | 4314.6 | 5.8 | 13.9 | 17.8 |
| | 1 | 6 | 0.4 | 4.9 | 266.1 | 424.0 | 543.0 | 25.0 | 28.3 | 152.1 |
| | 1 | 24 | 2.8 | 1.5 | 4.1 | 6.1 | 6.8 | 8.6 | 22.5 | 74.1 |
| | 1 | 48 | 0.9 | 0.9 | 5.6 | 1.8 | 1.5 | 1.9 | 1.8 | 26.3 |
| | 2 | 6 | 2.2 | 5.1 | 410.9 | 516.9 | 1681.6 | 13.6 | 53.2 | 130.1 |
| IL-10 | 1 | 3 | <3.2 | <3.2 | 15.6 | 30.4 | 27.3 | <3.2 | <3.2 | <3.2 |
| | 1 | 6 | <3.2 | <3.2 | <3.2 | 3.8 | 9.9 | <3.2 | <3.2 | 3.8 |
| | 1 | 24 | <3.2 | <3.2 | <3.2 | <3.2 | <3.2 | <3.2 | <3.2 | 3.8 |
| | 1 | 48 | <3.2 | <3.2 | <3.2 | <3.2 | <3.2 | <3.2 | <3.2 | <3.2 |
| | 2 | 6 | <3.2 | <3.2 | 1.2 | <3.2 | 7.7 | <3.2 | 1.2 | <3.2 |
| IL12p70 | 1 | 3 | <3.2 | 9.4 | <3.2 | <3.2 | 3.1 | <3.2 | <3.2 | 9.4 |
| | 1 | 6 | <3.2 | <3.2 | <3.2 | 3.1 | <3.2 | <3.2 | <3.2 | <3.2 |
| | 1 | 24 | <3.2 | <3.2 | <3.2 | <3.2 | <3.2 | <3.2 | <3.2 | 3.1 |
| | 1 | 48 | <3.2 | <3.2 | <3.2 | <3.2 | <3.2 | <3.2 | <3.2 | <3.2 |
| | 2 | 6 | <3.2 | <3.2 | <3.2 | 28.4 | 28.4 | <3.2 | <3.2 | 12.1 |
| IP10 | 1 | 3 | 88.9 | 124.3 | 1989.6 | 4826.2 | 8222.7 | 122.1 | 200.3 | 278.0 |
| | 1 | 6 | 95.4 | 116.3 | 8985.4 | 8828.3 | 7589.2 | 4294.0 | 5279.6 | 6945.6 |
| | 1 | 24 | 102.2 | 78.1 | 257.9 | 381.8 | 595.6 | 1123.5 | 1703.4 | 4251.8 |
| | 1 | 48 | 80.8 | 85.1 | 172.8 | 218.4 | 223.5 | 317.8 | 566.2 | 1631.2 |
| | 2 | 6 | 139.5 | 191.4 | >10000 | 3996.6 | 4151.5 | 1451.7 | 2590.8 | 2963.6 |
| MCP-1 | 1 | 3 | 17.3 | 65.0 | 333.3 | 1547.8 | 5805.3 | 21.8 | 48.1 | 50.2 |
| | 1 | 6 | 11.9 | 62.8 | 7790.8 | 12898.6 | 13861.5 | 518.9 | 311.8 | 1080.9 |
| | 1 | 24 | 24.4 | 2.0 | 53.0 | 141.5 | 143.9 | 283.5 | 694.9 | 5104.5 |
| | 1 | 48 | 29.3 | 19.4 | 56.8 | 40.0 | 2.0 | 118.0 | 108.0 | 530.5 |
| | 2 | 6 | 51.7 | 87.7 | 5244.6 | 9761.2 | 17185.9 | 175.5 | 338.2 | 592.4 |
| MIP1a | 1 | 3 | <3.2 | <3.2 | 10.3 | 251.5 | 1052.6 | <3.2 | <3.2 | 10.3 |
| | 1 | 6 | <3.2 | 10.3 | 101.9 | 137.2 | 172.2 | 10.3 | 10.3 | 100.5 |
| | 1 | 24 | 10.3 | <3.2 | <3.2 | <3.2 | 10.3 | <3.2 | <3.2 | 92.5 |
| | 1 | 48 | <3.2 | <3.2 | 10.3 | 10.3 | <3.2 | 10.3 | <3.2 | <3.2 |
| | 2 | 6 | 32.6 | 32.6 | 104.9 | 115.5 | 209.9 | 52.2 | 32.6 | 99.2 |
| MIP1b | 1 | 3 | <3.2 | <3.2 | 727.2 | 2471.0 | 7438.6 | <3.2 | <3.2 | <3.2 |
| | 1 | 6 | <3.2 | <3.2 | 670.7 | 1050.0 | 1162.7 | 155.7 | 256.7 | 695.2 |
| | 1 | 24 | <3.2 | <3.2 | <3.2 | <3.2 | <3.2 | <3.2 | <3.2 | 497.5 |
| | 1 | 48 | <3.2 | <3.2 | <3.2 | <3.2 | <3.2 | <3.2 | <3.2 | 157.8 |
| | 2 | 6 | <3.2 | <3.2 | 718.6 | 837.6 | 1407.7 | <3.2 | 216.1 | 449.7 |
| RANTES | 1 | 3 | <3.2 | <3.2 | <3.2 | 20.1 | 51.2 | <3.2 | 5.0 | <3.2 |
| | 1 | 6 | 20.3 | <3.2 | 130.3 | 255.0 | 526.2 | 14.7 | 17.1 | 23.2 |
| | 1 | 24 | <3.2 | <3.2 | 3.2 | 46.2 | 48.8 | 11.3 | 34.5 | 131.7 |
| | 1 | 48 | 9.2 | <3.2 | <3.2 | 5.0 | <3.2 | 7.9 | 17.1 | 79.4 |
| | 2 | 6 | 8.2 | 2.9 | 133.8 | 621.6 | 892.1 | 12.5 | 15.6 | 45.8 |
| TNFa | 1 | 3 | <3.2 | 4.3 | 9.1 | 21.0 | 46.4 | 3.2 | 3.2 | 4.5 |
| | 1 | 6 | <3.2 | 3.2 | 8.1 | 15.6 | 21.2 | 3.2 | 10.6 | 7.3 |
| | 1 | 24 | 7.3 | 3.2 | 3.2 | 5.2 | 3.2 | 5.2 | 5.9 | 15.2 |
| | 1 | 48 | <3.2 | 5.4 | 4.3 | <3.2 | 3.2 | 5.2 | 7.3 | 8.9 |
| | 2 | 6 | <3.2 | <3.2 | 10.0 | 16.7 | 46.4 | 3.2 | 3.2 | 3.2 |

The anti-tumor activity of the cleavable linker 11 conjugated to a non-binding mAb, PD-L1-targeted mAb (tumor and/or immune cell-targeted), or antigen C-targeted mAb (immune cell-targeted) was also evaluated in Renca tumor-bearing mice. All conjugates demonstrated tumor growth delay compared to untreated tumors. The PD-L1-targeted mAb conjugate of 11 demonstrated enhanced anti-tumor activity compared to an unconjugated PD-L1-targeted mAb. This demonstrates the anti-tumor benefit of delivering STING agonists using an ADC targeting antigens C and PD-L1 (FIG. 13). The anti-tumor activity of the non-cleavable linker 12 conjugated to a PD-L1-targeted mAb was also evaluated in Renca tumor-bearing mice; these conjugates were efficacious at reducing tumor volume, though less well tolerated than PD-L1 targeted mAb conjugates of 11.

CT26 Cancer Cells

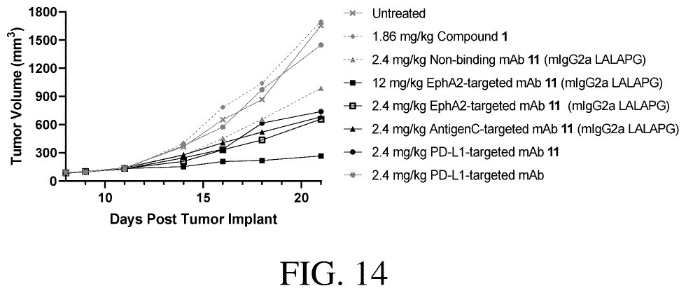

The anti-tumor activity of compound 1 compared to the cleavable linker 11 conjugated to a non-binding mAb, antigen C-targeted mAb, PD-L1-targeted mAb, or EphA2-targeted mAb was evaluated in CT26 tumor-bearing mice. When animals were treated with compound 1 or the unconjugated PD-L1-targeted mAb, minimal tumor growth delay was observed. Modest tumor growth delay was observed with the non-binding mAb conjugate of 11. In contrast, significant tumor growth delay was observed following treatment with all three targeted mAb conjugates of 11. This demonstrates the anti-tumor benefit of delivering STING agonists using an ADC targeting a variety of antigens, including an immune cell-targeted conjugate (antigen C), immune and/or tumor-targeted conjugate (PD-L1), and tumor-targeted conjugate (EphA2) (FIG. 14). Results of cytokine production in peripheral blood plasma is presented in Table 7.

TABLE 7
Cytokine production in peripheral blood (plasma) in CT26 tumor-bearing mice
upon treatment with various ADCs comprising a mAb conjugated to compound 11.

| | Time | | | | | | | 12 mg/kg EphA2-targeted | | | 2.4 mg/kg EphA2-targeted | | |
|---|---|---|---|---|---|---|---|---|---|---|---|---|---|
| | (h) | Untreated | | | 1.86 mg/kg Compound 1 | | | mAb 11 (mIgG2a LALAPG) | | | mAb 11 (mIgG2a LALAPG) | | |
| IL-6 | 3 | 0.5 | 3.2 | 3.2 | 622.1 | 781.4 | 1128.3 | 185.8 | 0.5 | 217.7 | 17.8 | 27.7 | 23.4 |
| | 6 | 2.7 | 3.2 | 0.2 | 2652.5 | 2393.5 | 2412.7 | 2677.9 | 1790.2 | 2777.7 | 168.7 | 303.4 | 304.8 |
| | 24 | 5.7 | 5.4 | 0.6 | 44.5 | 31.8 | 36.5 | 72.8 | 0.2 | 67.6 | 16.2 | 143.2 | 16.1 |
| | 48 | 26.5 | 262.8 | 7.6 | 47.8 | 142.7 | 29.2 | 214.1 | 61.7 | 80.4 | 6.5 | 39.1 | 7.8 |
| IL-10 | 3 | 3.2 | 3.2 | 3.2 | 0.2 | 2.3 | 1.2 | 3.2 | 3.2 | 1.2 | 3.2 | 3.2 | 3.2 |
| | 6 | 3.2 | 3.2 | 3.2 | 7.8 | 9 | 4.4 | 7.2 | 2 | 4.3 | 4 | 1.2 | 1.6 |
| | 24 | 3.2 | 3.2 | 3.2 | 3.2 | 3.2 | 3.2 | 3.2 | 3.2 | 1.5 | 3.2 | 3.2 | 3.2 |
| | 48 | 3.2 | 3.2 | 3.2 | 3.2 | 3.2 | 3.2 | 3.2 | 3.2 | 3.2 | 3.2 | 3.2 | 3.2 |
| IP-10 | 3 | 242.4 | 109.7 | 200.2 | 1439.4 | 1501.7 | 1160.7 | 592.6 | 125.9 | 377.2 | 198.1 | 190.9 | 196.9 |
| | 6 | 174.7 | 117.4 | 188.2 | 2610.8 | 2133.6 | 2473.4 | 2039.3 | 2711.3 | 2008.6 | 2327.7 | 2740.5 | 1949.2 |
| | 24 | 272.5 | 134.4 | 201.1 | 1121.6 | 1025.3 | 696.2 | 1468.5 | 145 | 1900.5 | 1189.9 | 1094.8 | 1089 |
| | 48 | 122.9 | 155.8 | 74.7 | 560.1 | 398.8 | 560 | 1293.4 | 541.3 | 716.9 | 307.2 | 167.2 | 250.2 |
| MCP-1 | 3 | 2.7 | 3.2 | 3.2 | 73.7 | 78.6 | 110.3 | 36.4 | 2.7 | 23.1 | 3.2 | 10.5 | 2.7 |
| | 6 | 7.2 | 7.2 | 3.2 | 3185.3 | 2816.3 | 1634.8 | 6186.6 | 3754.5 | 3698.6 | 656.5 | 578.3 | 382.2 |
| | 24 | 3.2 | 3.2 | 3.2 | 397.7 | 294.7 | 305.9 | 765.3 | 3.2 | 821 | 99.1 | 99.1 | 99.1 |
| | 48 | 13.5 | 18.6 | 3.2 | 93.1 | 20.9 | 41.4 | 207.4 | 85.1 | 108.7 | 27.2 | 10.5 | 13.5 |
| MIP-1a | 3 | 3.2 | 3.2 | 3.2 | 3.9 | 3.2 | 3.2 | 3.9 | 3.2 | 3.2 | 3.2 | 3.2 | 3.2 |
| | 6 | 3.9 | 3.2 | 3.2 | 94.6 | 93.6 | 73.9 | 222.1 | 127.3 | 261.8 | 12.7 | 3.9 | 3.2 |
| | 24 | 3.2 | 3.2 | 3.2 | 12.7 | 25.1 | 3.2 | 21.7 | 3.2 | 21.7 | 3.2 | 3.2 | 3.2 |
| | 48 | 3.2 | 3.2 | 3.2 | 3.9 | 12.7 | 3.2 | 3.9 | 3.2 | 3.9 | 3.2 | 3.2 | 3.2 |
| MIP1b | 3 | 3.2 | 1.6 | 3.2 | 297.7 | 336.8 | 224 | 311.6 | 3.2 | 185.9 | 26.9 | 19.7 | 5.4 |
| | 6 | 3.2 | 3.2 | 3.2 | 925.8 | 369.7 | 1161.4 | 1679.2 | 1460.7 | 1237.9 | 367.2 | 264.6 | 356.3 |
| | 24 | 3.2 | 3.2 | 3.2 | 127.6 | 125.3 | 55.3 | 268.8 | 3.2 | 356.8 | 84.9 | 63.2 | 51.3 |
| | 48 | 3.2 | 14.8 | 3.2 | 61.1 | 10.8 | 57.6 | 66.7 | 26.9 | 56.9 | 11.4 | 3.2 | 8.8 |
| TNFa | 3 | 3.2 | 3.2 | 3.2 | 3.2 | 4.9 | 4.3 | 1.4 | 3.2 | 3.2 | 3.2 | 3.2 | 3.2 |
| | 6 | 3.2 | 3.2 | 3.2 | 16.9 | 16.6 | 10 | 29.1 | 26.2 | 34.8 | 6.2 | 3.8 | 2.6 |
| | 24 | 3.2 | 3.2 | 3.2 | 0.4 | 0.7 | 3.2 | 4.3 | 3.2 | 3.2 | 3.2 | 2.9 | 8.5 |
| | 48 | 3.2 | 3.2 | 3.2 | 1.1 | 3.2 | 3.2 | 1.4 | 3.2 | 3.2 | 3.2 | 3.2 | 3.2 |

| | 2.4 mg/kg Antigen C-targeted | | | 2.4 mg/kg Non-binding | | | 2.4 mg/kg PD-L1- | | | 2.4 mg/kg PD-L1- | | |
|---|---|---|---|---|---|---|---|---|---|---|---|---|
| | mAb 11 (mIgG2a LALAPG) | | | mAb 11 (mIgG2a LALAPG) | | | targeted mAb 11 | | | targeted mAb | | |
| IL-6 | 483.9 | 182.4 | 442.5 | 1.4 | 100.6 | 56.5 | 18.1 | 56.9 | 3.3 | 25.6 | 0.6 | 5.8 |
| | 1821.4 | 2769.8 | 2324.8 | 123.7 | 151.3 | 266.9 | 119.4 | 72.6 | 162.5 | 3.2 | 3.2 | 1 |
| | 9.4 | 34.4 | 126.9 | 5 | 16.9 | 3.2 | 23.8 | 21.8 | 5.2 | 21.2 | 6.5 | 216.8 |
| | 48.9 | 32.2 | 19.5 | 2.9 | 2.9 | 2.3 | 26.6 | 26.8 | 89.7 | 1.4 | 3.2 | 12.2 |
| IL-10 | 0.9 | 3.2 | 0.4 | 3.2 | 3.2 | 3.2 | 3.2 | 3.2 | 3.2 | 33.8 | 3.2 | 3.2 |
| | 1.2 | 3.7 | 4.3 | 0.4 | 0.4 | 2 | 0.4 | 0 | 0 | 3.2 | 3.2 | 3.2 |
| | 0.4 | 3.2 | 3.2 | 3.1 | 3.2 | 3.2 | 3.2 | 3.2 | 3.2 | 26.2 | 3.2 | 3.2 |
| | 3.2 | 3.2 | 3.2 | 3.2 | 3.2 | 3.2 | 3.2 | 3.2 | 0.4 | 3.2 | 3.2 | 3.2 |
| IP-10 | 1271.3 | 629.1 | 627.8 | 109.4 | 450 | 308.7 | 163.9 | 154.4 | 178.3 | 105.7 | 190.4 | 96.1 |
| | 2062.9 | 2328 | 2713.5 | 1779.6 | 1376.6 | 2092 | 1814.4 | 1744 | 1389.4 | 130.6 | 151.9 | 110.7 |
| | 2565 | 1994.5 | 1329.8 | 246.4 | 693.7 | 3.2 | 717.5 | 1002.6 | 833.1 | 247.7 | 689.9 | 166.7 |
| | 448.8 | 273.5 | 378 | 107.3 | 235.9 | 319.2 | 748.1 | 599.3 | 580.4 | 128.1 | 183.3 | 135.9 |
| MCP-1 | 393.2 | 69.9 | 78.6 | 3.2 | 46.2 | 2.7 | 18.9 | 13.8 | 3.2 | 169.8 | 3.2 | 3.2 |
| | 10207.3 | 4960.7 | 37687.1 | 162 | 556.2 | 425 | 143.3 | 133 | 252 | 3.1 | 7.5 | 7.5 |
| | 1009.2 | 637.8 | 194 | 3.2 | 55.1 | 3.2 | 129.2 | 191.4 | 98 | 129.2 | 7.5 | 3.2 |
| | 39.8 | 34.7 | 365.9 | 3.2 | 2.7 | 20.9 | 135.9 | 127.3 | 253.1 | 7.5 | 13.8 | 16.4 |
| MIP-1a | 217.7 | 103.1 | 139.8 | 3.2 | 3.2 | 3.2 | 3.2 | 3 | 3.2 | 3 | 3 | 3 |
| | 144 | 152.7 | 82.1 | 17.7 | 12.7 | 3.9 | 11.2 | 3 | 3 | 3 | 3 | 11.2 |
| | 17.7 | 21.7 | 12.7 | 3.2 | 3.2 | 3.2 | 3 | 3 | 3 | 3 | 11.2 | 24 |
| | 3.2 | 3.2 | 17.7 | 3.2 | 3.2 | 3.2 | 11.2 | 11.2 | 3 | 3 | 7.9 | 3 |
| MIP1b | 1600.5 | 682.1 | 991.5 | 3.2 | 31.2 | 7.2 | 3.2 | 5.4 | 9.9 | 3.2 | 3.2 | 3.2 |
| | 1377.1 | 1236.6 | 1740.4 | 137.2 | 215.4 | 203.6 | 70 | 61.7 | 85.7 | 3.2 | 3.2 | 3.2 |
| | 197.4 | 142.2 | 127.5 | 3.2 | 26.9 | 3.2 | 3.2 | 19.3 | 32 | 3.2 | 3.2 | 46.1 |
| | 13.8 | 13.2 | 69 | 3.2 | 3.2 | 3.2 | 26.5 | 23.5 | 30 | 3.2 | 3.2 | 3.2 |
| TNFa | 31 | 25.6 | 27.6 | 3.2 | 3.2 | 3.2 | 5.8 | 3.2 | 3.2 | 75.5 | 3.2 | 3.2 |
| | 31 | 45.5 | 39.5 | 3.2 | 4.3 | 3.2 | 3.2 | 3.2 | 3.2 | 3.2 | 3.2 | 3.2 |
| | 2 | 7 | 3.8 | 3.2 | 1.4 | 3.2 | 6.4 | 3.2 | 3.2 | 58.6 | 3.2 | 3.2 |
| | 3.2 | 3.2 | 1.7 | 3.2 | 3.2 | 3.2 | 3.6 | 3.2 | 3.2 | 3.2 | 3.2 | 3.2 |

MC38 Cancer Cells

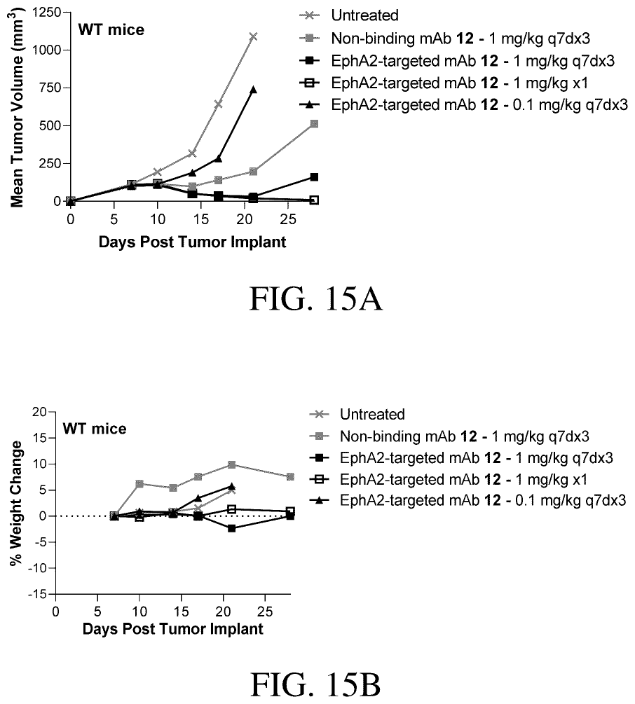

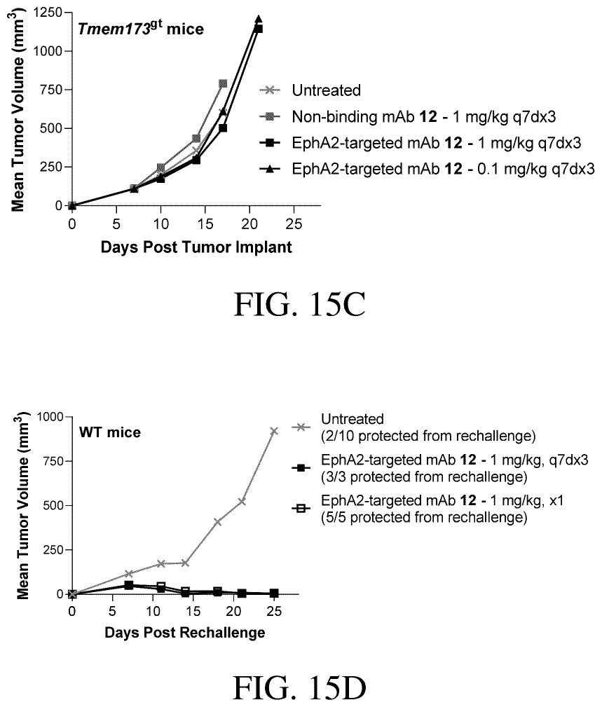

The anti-tumor activity of the cleavable linker 12 conjugated to a non-binding mAb or EphA2-targeted mAb with a LALAPG mIgG2a Fc backbone was evaluated in MC38-tumor bearing wild type (WT) or STING-deficient (Tmem173^{gt}) mice. Animals treated with 3 weekly doses of 1 mg/kg non-binding conjugates of 12 or 0.1 mg/kg targeted conjugates of 12 demonstrated modest and minimal tumor growth delay, respectively, in WT but not STING-deficient tumor bearing mice. Animals treated with 3 weekly doses of 1 mg/kg targeted conjugates of 12 demonstrated robust tumor growth delay in WT but not STING-deficient tumor bearing mice. This demonstrates that in MC38 tumor-bearing mice STING signaling is required in non-tumor cells in the tumor microenvironment for anti-tumor activity (FIGS. 15A and 15C).

Animals treated with a single dose of 1 mg/kg EphA2-targeted conjugates of 12 also demonstrated robust tumor growth delay in WT tumor bearing mice, demonstrating that a single dose of EphA2-targeted conjugates of 12 is sufficient to drive complete tumor regression (FIG. 15A).

Mice that achieved complete tumor regression in response to a single dose or 3 weekly doses of ADC were rechallenged with MC38 tumor cells on the opposite flank and tumor growth was monitored. All rechallenged mice—but not all naïve untreated mice challenged with MC38 tumor cells—were protected from rechallenge, suggesting that targeted conjugates of 12 elicit immune memory (FIG. 15D).

4T1 Cancer Cells

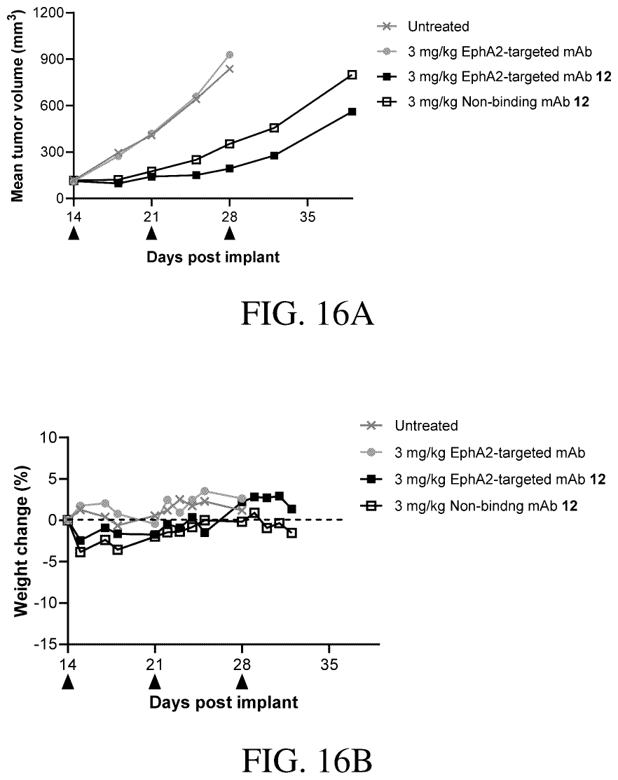

The anti-tumor activity of the cleavable linker 12 conjugated to a non-binding or EphA2-targeted mAb with a LALAPG mIgG2a Fc backbone was evaluated in 4T1 tumor-bearing mice. All conjugates of compound 12 led to significant tumor growth delay at the doses tested, with the targeted mAb conjugate of compound 12 demonstrating enhanced tumor growth delay compared to the non-binding conjugate, with minimal weight loss observed (FIG. 16B). This demonstrates that EphA2-targeted mAb conjugates of compound 12 are active in multiple tumor models (FIGS. 12-16).

### Rat Tolerability Study:

The nonclinical safety profile of compound 12 conjugated to non-binding antibodies with a WT Fc backbone, non-binding antibodies with an Fc null backbone, targeted antibodies with a WT Fc backbone, and targeted antibodies with an Fc null backbone was evaluated in non-GLP rat toxicology studies. All conjugates with the compound 12 drug linker (both non-binding and targeted, WT and null Fc backbone) were tolerated in rat at doses higher than the minimally efficacious dose in mouse tumor models.

### Example 4

### In Vivo Pharmacokinetic Study

### Methods

Pharmacokinetic profiles were analyzed following administration of two weekly 1 mg/kg doses of an ADC comprising a [deglycosylated] non-binding mAb conjugated to compound 12 to male C57BL/6 mice. Plasma was collected and analyzed for generic total antibody (gTAb) by immunoassay. TAb concentrations in mouse K₂EDTA plasma were determined by a Gyros flow-through immunoassay platform. Samples and standards were diluted in assay buffer and incubated with a solution containing biotinylated murine anti-human kappa light chain antibody and fluorescent goat anti-human IgG Fcg F(ab′)₂ antibody fragment in a sandwich format. The resulting immunocomplexes were bound to the streptavidin-coated beads in the affinity column of the compact disc (CD). The CD was read by a laser that excites the fluorescent detection reagent, producing a signal that is directly proportional to the concentration of test article in the C57BL/6 male mouse plasma sample. Non-compartmental analysis was applied to pooled animal plasma concentration data (sparse sampling) using Phoenix WinNonlin 8.2 (Certara, USA). Concentration values below the limit of quantitation (BLQ) were treated as zero for analysis. Nominal doses and sampling times were used.

### Results

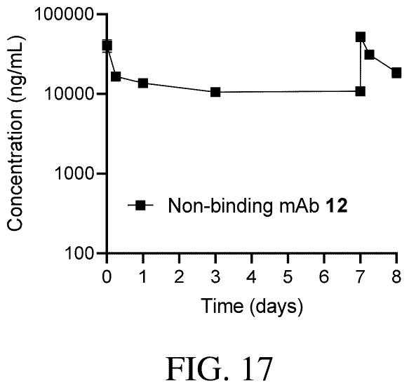

Pharmacokinetic profiles were analyzed following administration of two weekly 1 mg/kg doses of an ADC comprising a [deglycosylated] non-binding mAb conjugated to compound 12 to male C57BL/6 mice. The maximum observed concentration (Cmax) after the first and second dose was 40500 and 52400 ng/mL, respectively. The area under the concentration-time curve from time 0 through 7 days (AUC0-7d) was 85600 d*ng/mL. This suggests that the total antibody exposure for the non-binding conjugate of compound 12 was higher than the small molecule exposure of published small molecule STING agonists (FIG. 17)(See, e.g., Ramanjulu et al., 2018, Nature 564, 439-443).

The contents of each of the references cited in the present disclosure are hereby incorporated by reference in their entirety.

A number of embodiments of the present disclosure have been described. Nevertheless, it will be understood that various modifications may be made without departing from the spirit and scope of the present disclosure. Accordingly, other embodiments are within the scope of the following claims.

1. A compound of Formula (II): 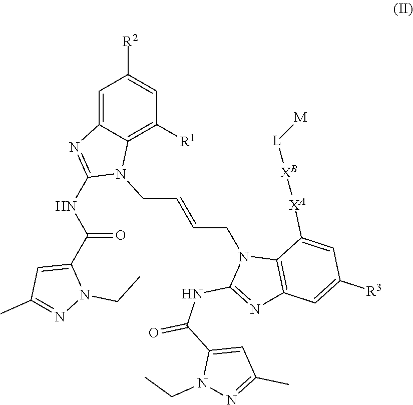
or a pharmaceutically acceptable salt thereof, wherein:
R¹ is hydrogen, hydroxyl, C₁₋₆ alkoxy, —(C₁₋₆ alkyl) C₁₋₆ alkoxy, —(CH₂)ₙ—NR^{A}R^{B}, or PEG2 to PEG4;
each R² and R³ are independently —CO₂H, —(C═O)ₘ—NR^{C}R^{D}, or —(CH₂)_{q}—NR^{E}R^{F};
each R^{A}, R^{B}, R^{C}, R^{D}, R^{E}, and R^{F} are independently hydrogen or C₁₋₃ alkyl;
each subscript n is independently an integer from 0 to 6;
each subscript m is independently 0 or 1;
each subscript q is independently an integer from 0 to 6;
X^{A} is —CH₂—, —O—, —S—, —NH—, or —N(CH₃)—;
X^{B} is absent or a 2-16 membered heteroalkylene;
L is a linker having the formula -(A)ₐ-(W)_{w}-(Y)_{y}—, wherein:
subscript a is 0 or 1;
subscript y is 0 or 1;
subscript w is 0 or 1;
A is a C₂₋₂₀ alkylene optionally substituted with 1-3 R^{a1}; or a 2 to 40 membered heteroalkylene optionally substituted with 1-3 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —C(O)NR^{d1}R^{e1}, —C(O)(C₁₋₆ alkyl), and —C(O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —C(O)NR^{d1}R^{e1}, —C(O)(C₁₋₆ alkyl), and —C(O)O(C₁₋₆ alkyl);
each R^{d1} and R^{e1} are independently hydrogen or C₁₋₃ alkyl;
W is from 1-12 amino acids or has the structure: 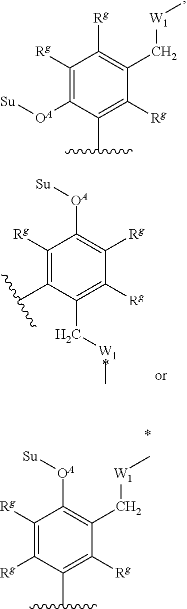
wherein Su is a Sugar moiety;
—O^{A}— represents a glycosidic bond;
each R^{g} is independently hydrogen, halogen, —CN, or —NO₂;
W¹ is absent or —O—C(═O)—; [symbol]
represents covalent attachment to A or M;
* represents covalent attachment to Y, X^{A}, or X^{B}; and
Y is a self-immolative moiety, a non-self-immolative releasable moiety, or a non-cleavable moiety;
M is 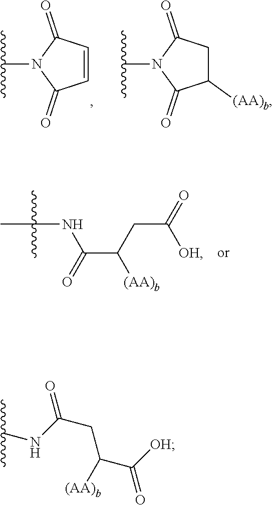
each AA is an independently selected amino acid, wherein (AA)_{b} is connected to the succinimide or hydrolyzed succinimide via a sulfur atom;
each subscript b is independently an integer from 1 to 6; and
X^{B} and L are each independently optionally substituted with a PEG Unit from PEG1 to PEG 72.

2-35. (canceled)

36. The compound of claim 1, wherein X^{A} is —O—.

37-68. (canceled)

69. The compound of claim 1, wherein each amino acid in W is independently selected from the group consisting of alanine, glycine, lysine, serine, aspartic acid, aspartate methyl ester, N,N-dimethyl-lysine, phenylalanine, citrulline, valine-alanine, valine-citrulline, phenylalanine-lysine or homoserine methyl ether.

70-84. (canceled)

85. The compound of claim 1, wherein A is a 4 to 12 membered heteroalkylene.

86-90. (canceled)

91. The compound of claim 1, wherein

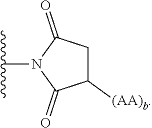

92-104. (canceled)

105. The compound of claim 1, selected from the group consisting of: 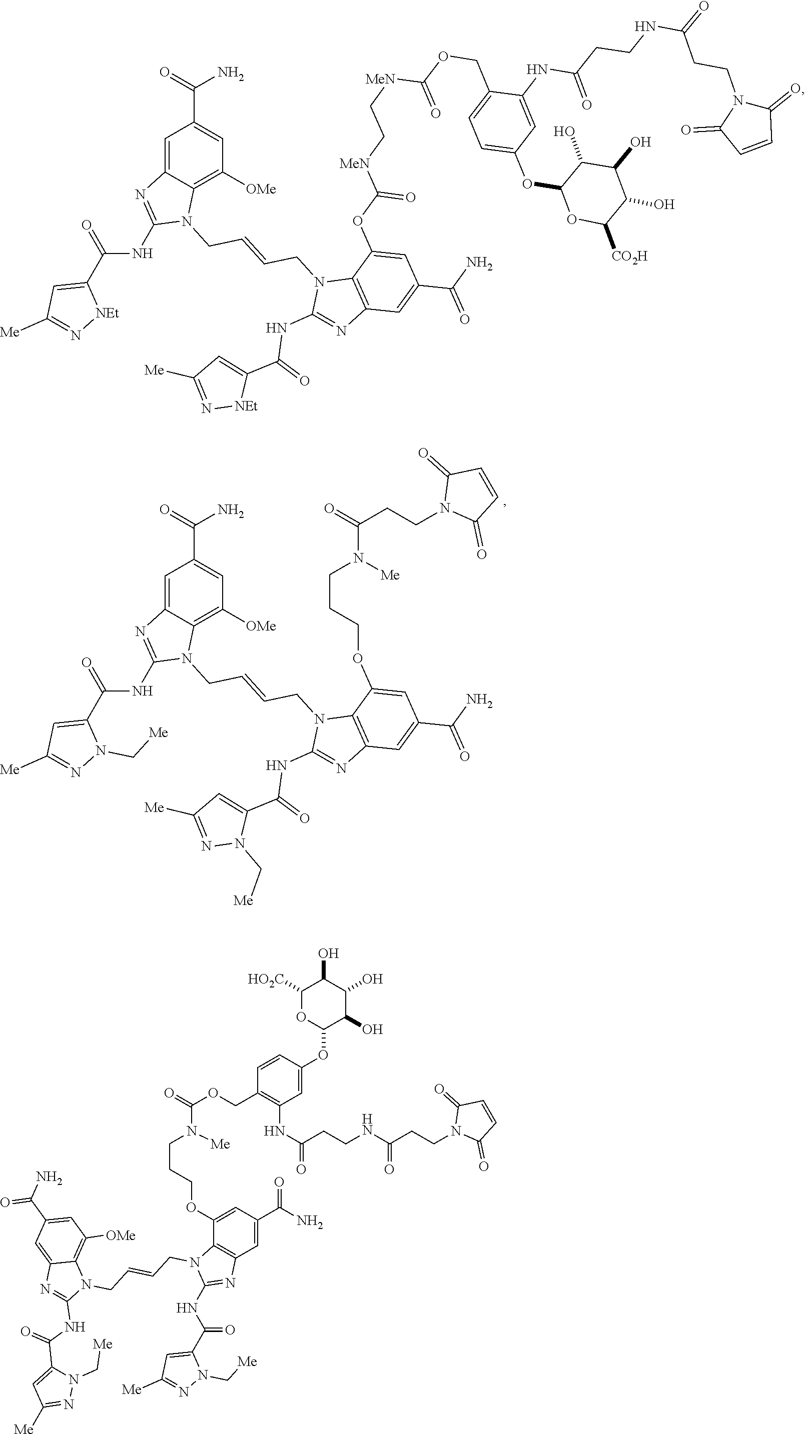 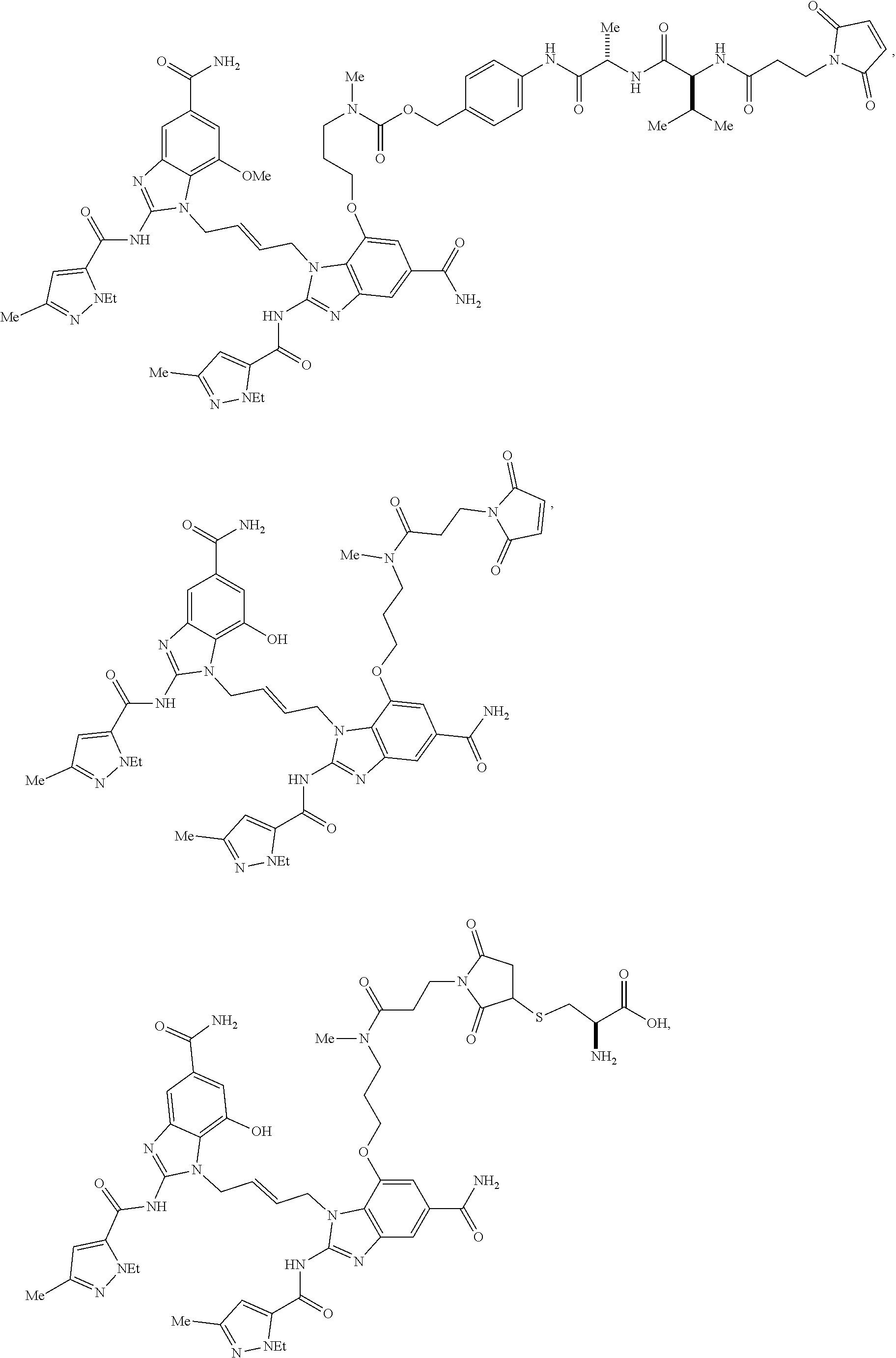 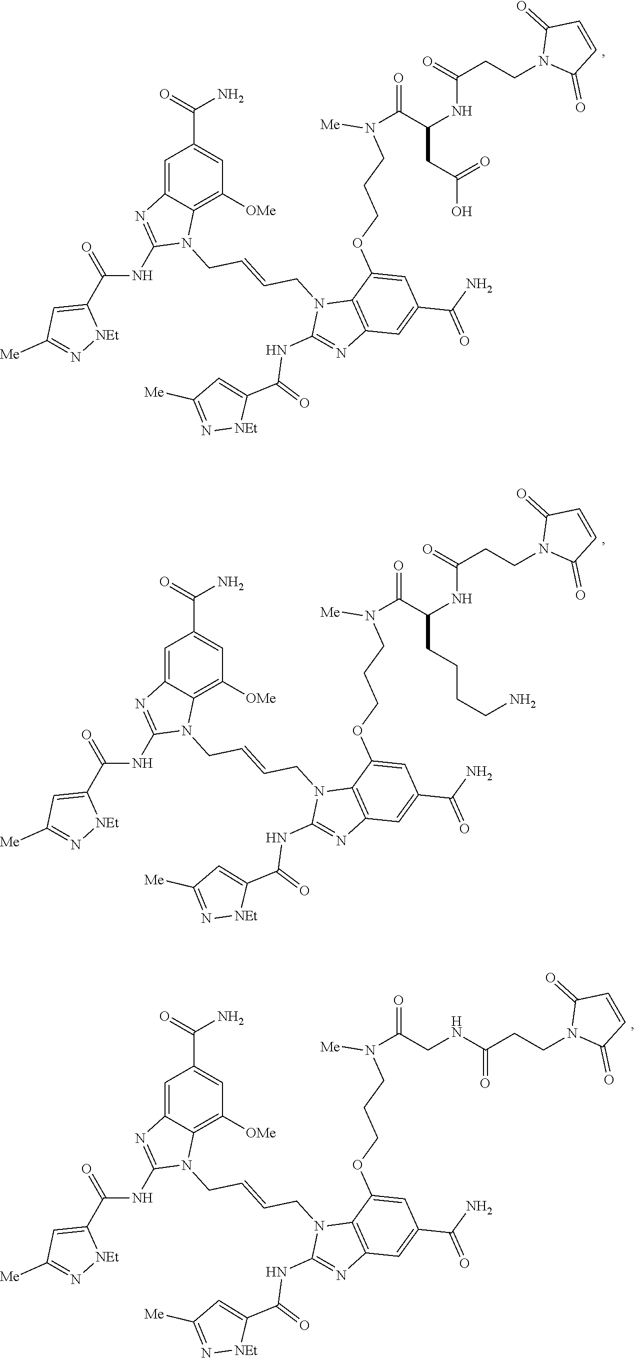 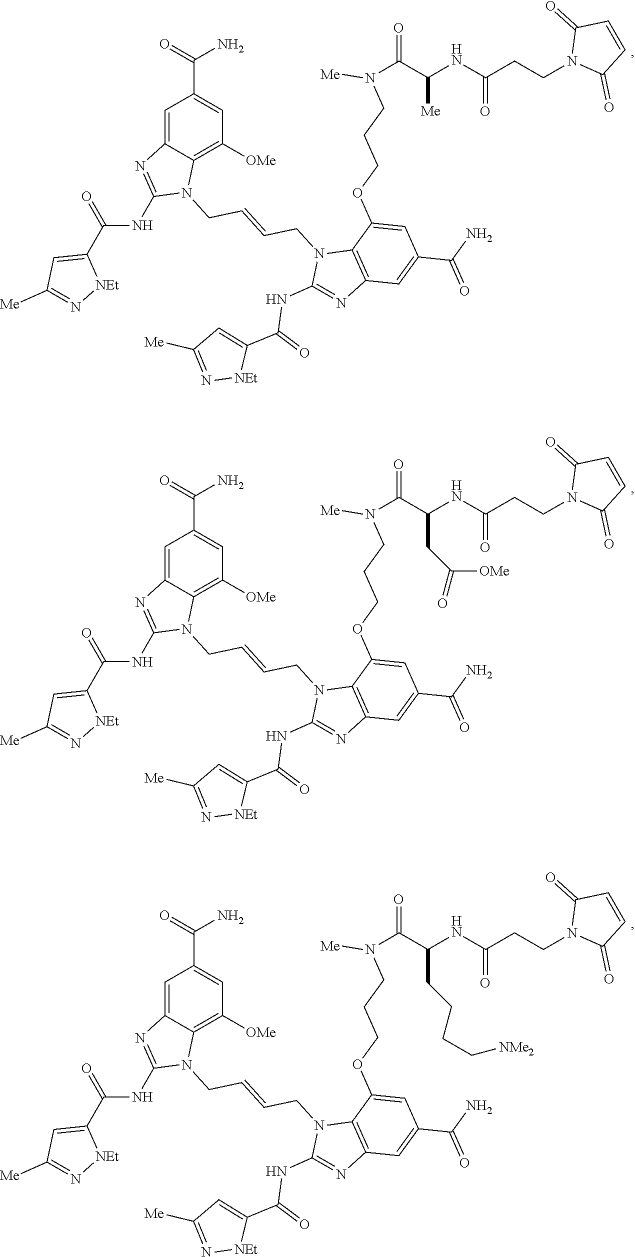 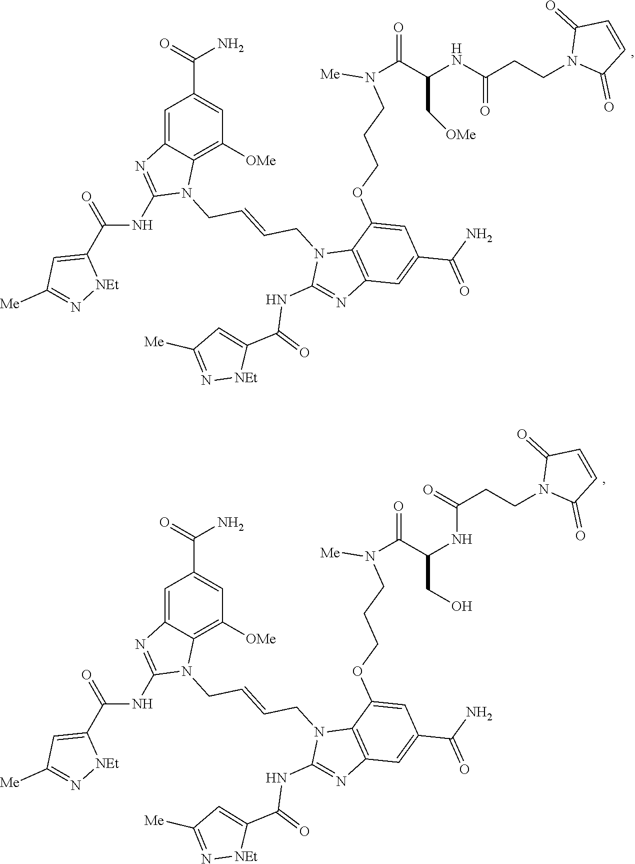
and pharmaceutically acceptable salts thereof.

106. The compound of claim 1, having the structure of Formula (II-A): 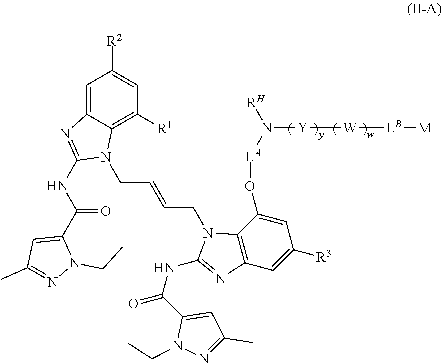
or a pharmaceutically acceptable salt thereof, wherein:
L^{A} is —(CH₂)₁₋₆—, —C(O)(CH₂)₁₋₆—, or —C(O)NR^{H}(CH₂)₁₋₆—;
each R^{H} is independently hydrogen or C₁₋₃ alkyl;
Y is 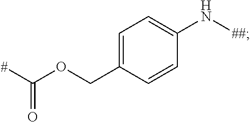
# represents covalent attachment to —NR^{H}L^{A};
## represents covalent attachment to W or L^{B}; and
L^{B} is —(CH₂)₁₋₆—, —C(O)(CH₂)₁₋₆—, or —[NHC(O)(CH₂)₁₋₄]₁₋₃—.

107. The compound of claim 106, wherein R^{H} is methyl.

108-112. (canceled)

113. The compound of claim 106, wherein each amino acid of W is independently selected from the group consisting of alanine, valine, isoleucine, leucine, aspartic acid, glutamic acid, lysine, histidine, arginine, glycine, serine, threonine, phenylalanine, O-methylserine, O-methylaspartic acid, O-methylglutamic acid, N-methyllysine, O-methyltyrosine, O-methylhistidine, and O-methylthreonine.

114-116. (canceled)

117. The compound of claim 106, selected from the group consisting of: 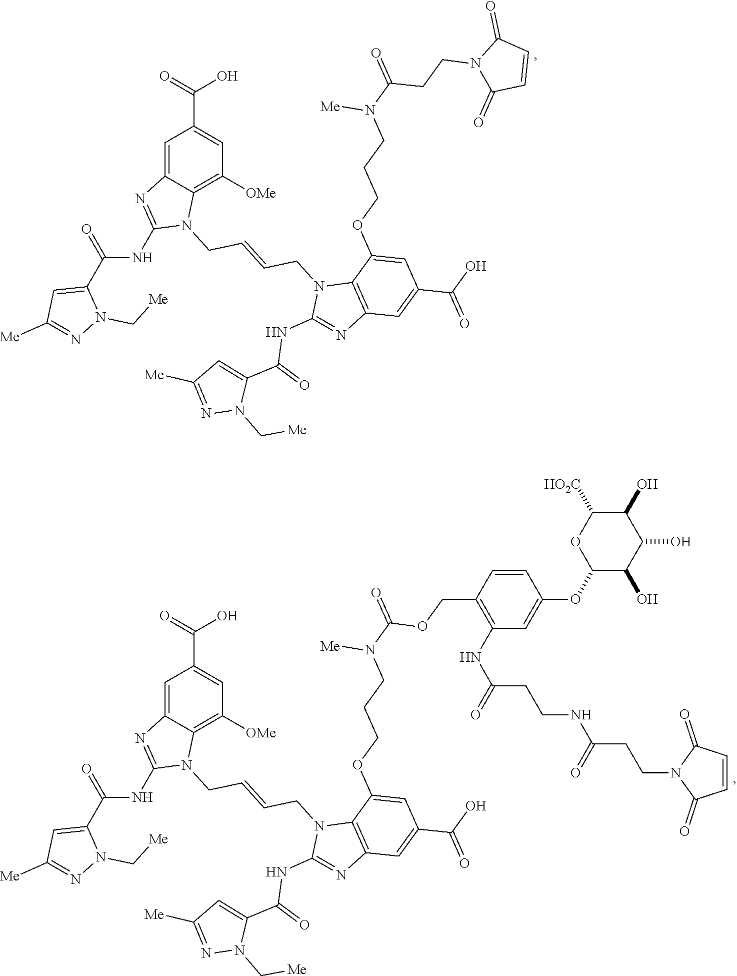 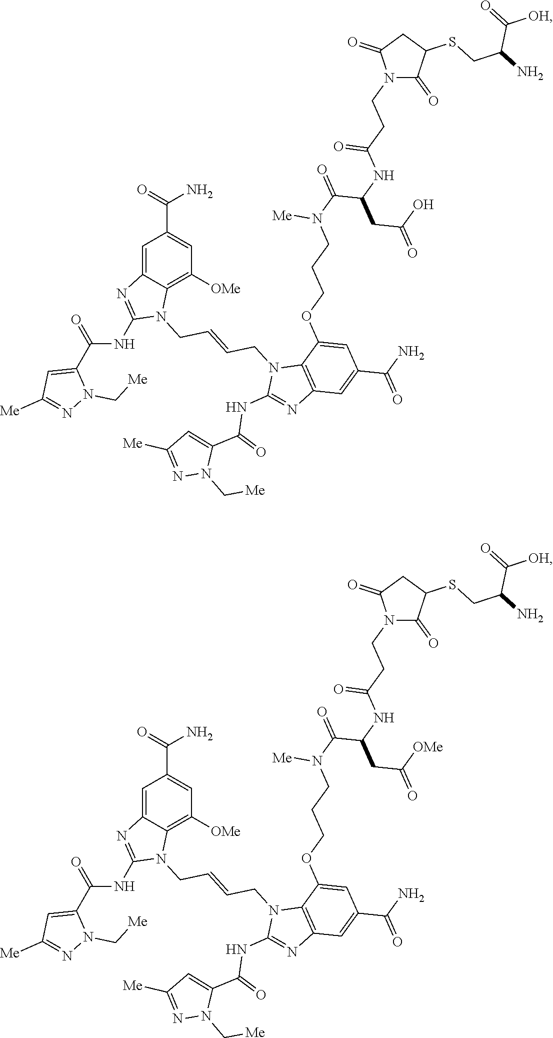 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CC(=O)O)NC(=O)CCN3C(=O)CC(SC[C@H](N)C(=O)O)C3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CC(=O)OC)NC(=O)CCN3C(=O)CC(SC[C@H](N)C(=O)O)C3=O)c21] 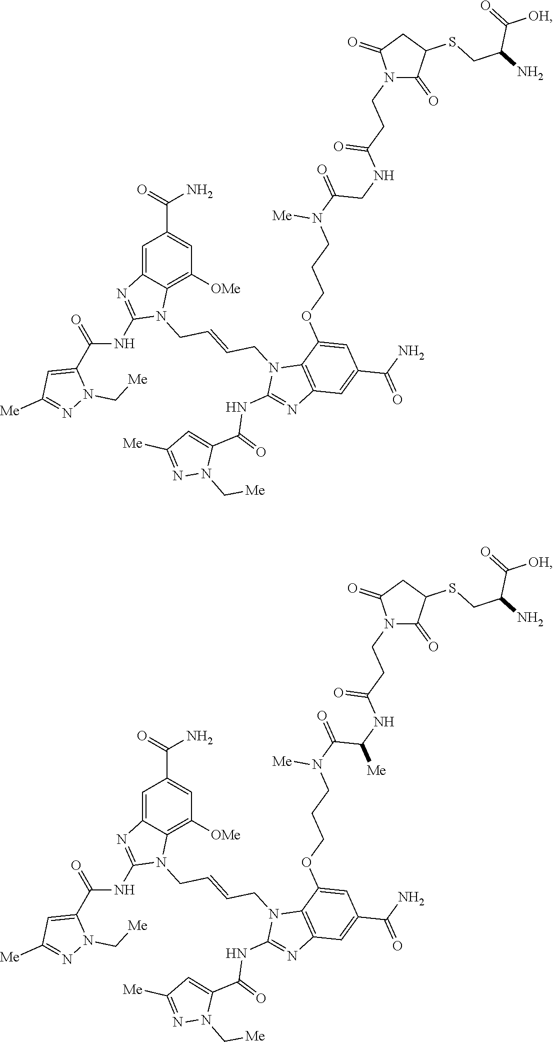 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)CNC(=O)CCN3C(=O)CC(SC[C@H](N)C(=O)O)C3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](C)NC(=O)CCN3C(=O)CC(SC[C@H](N)C(=O)O)C3=O)c21] 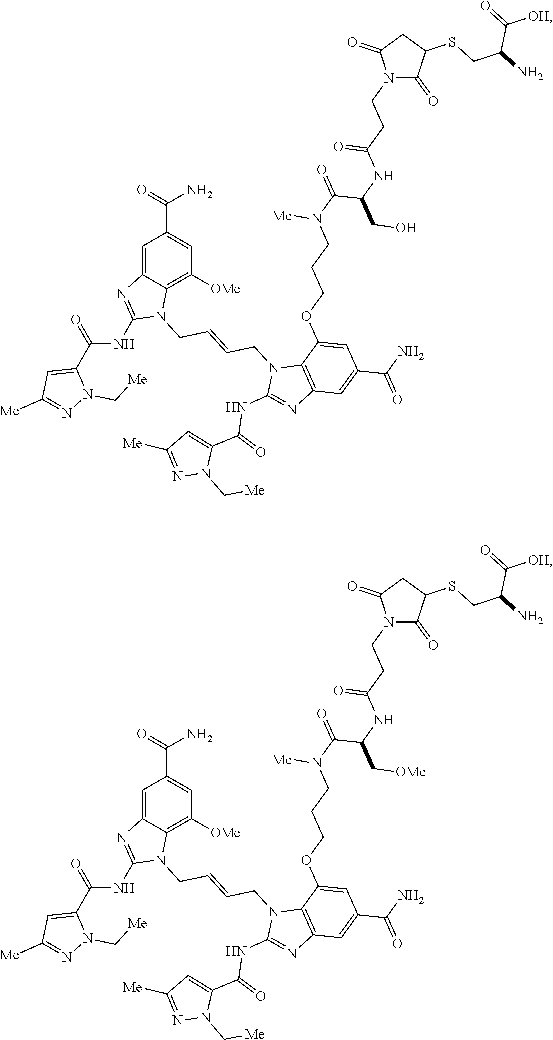 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CO)NC(=O)CCN3C(=O)CC(SC[C@H](N)C(=O)O)C3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](COC)NC(=O)CCN3C(=O)CC(SC[C@H](N)C(=O)O)C3=O)c21] 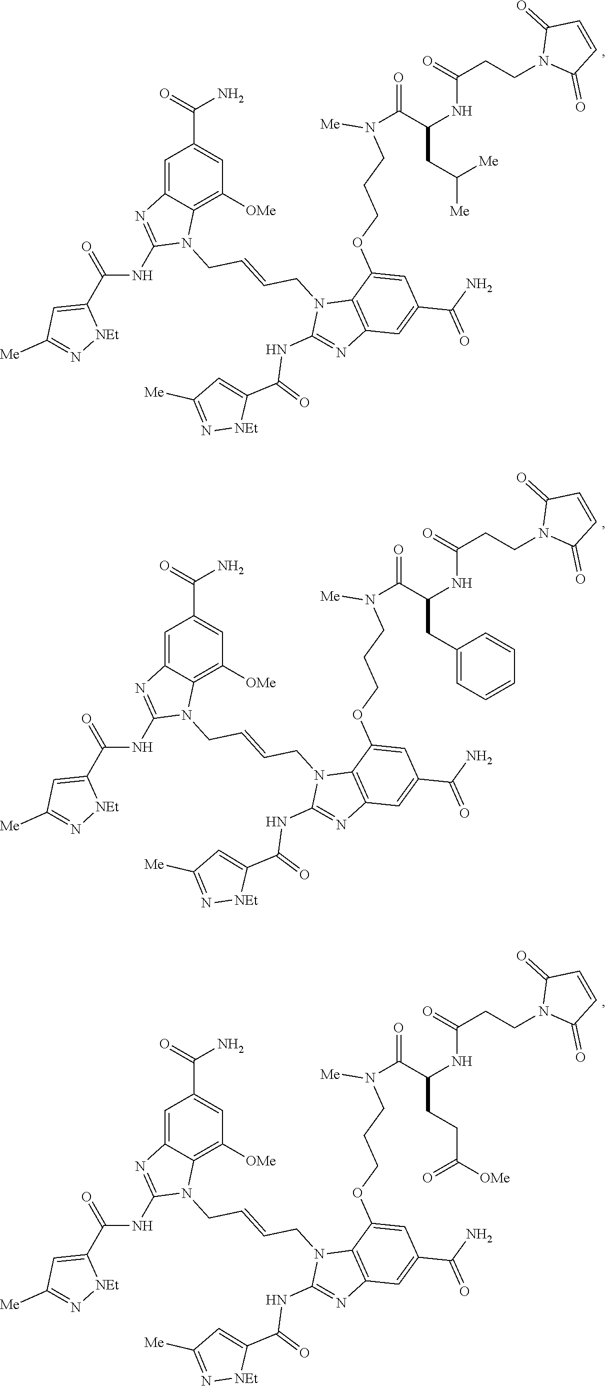 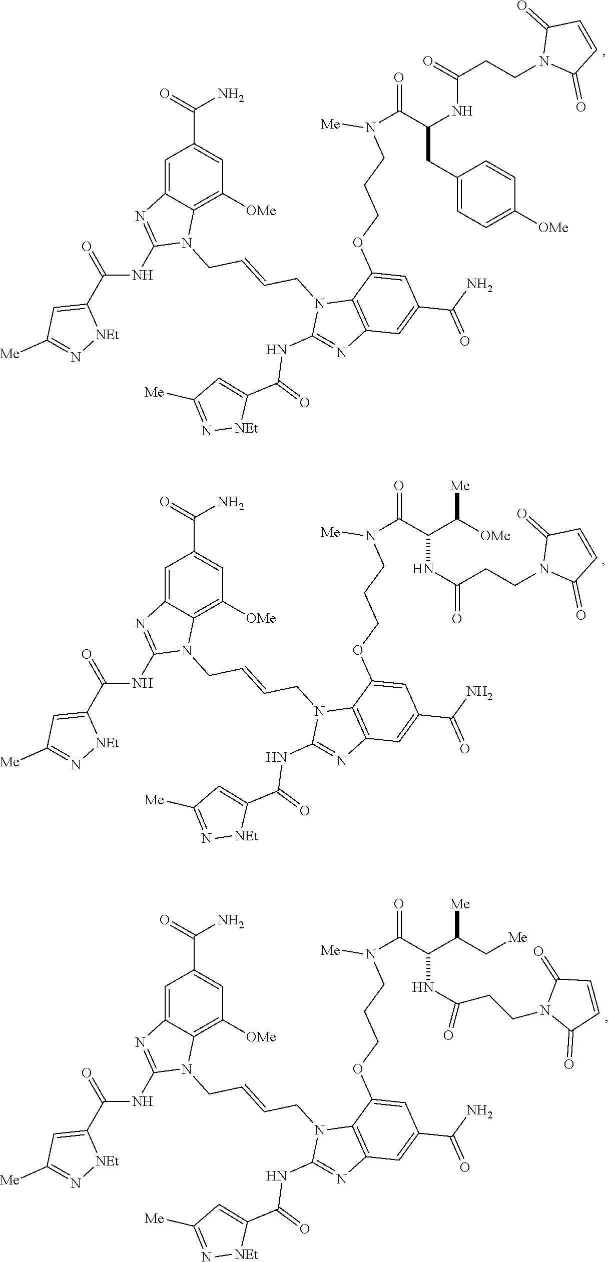 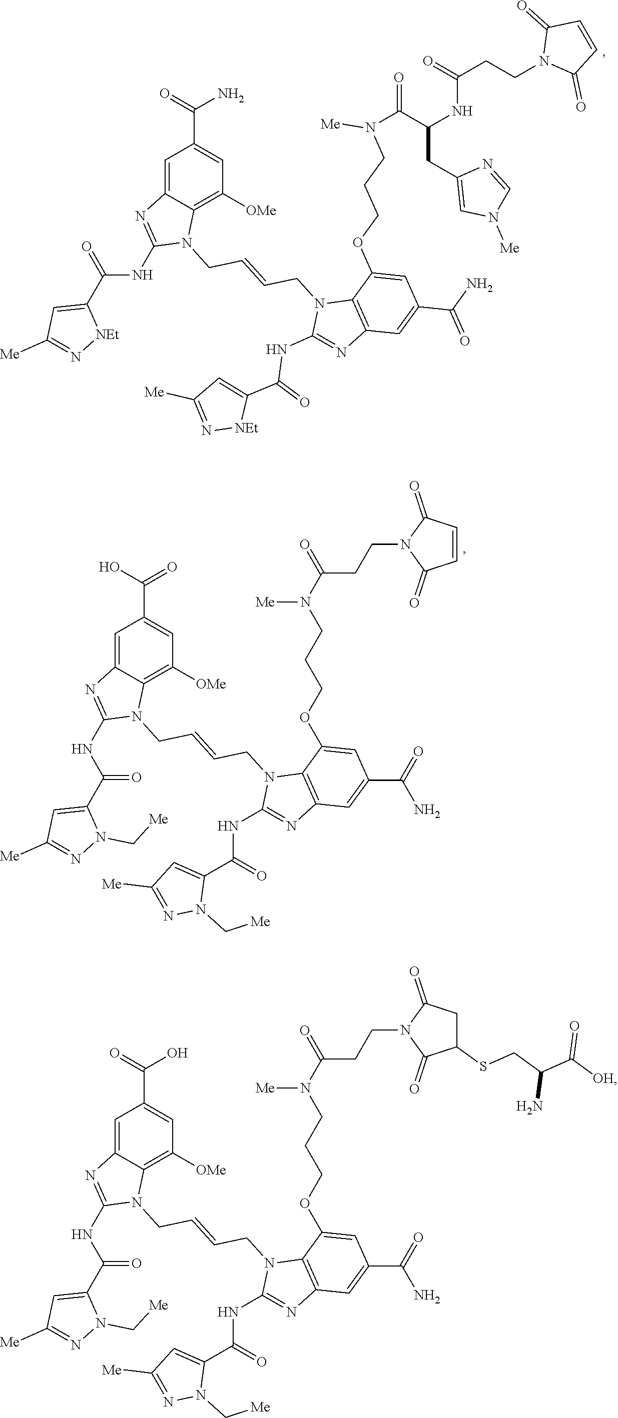 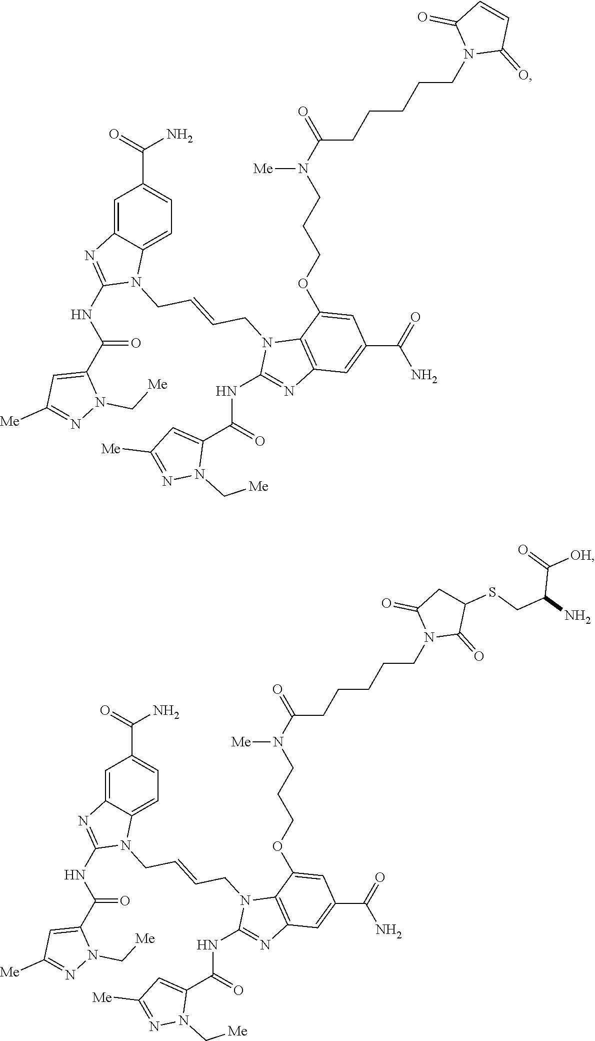 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN(C)C(=O)CCCCCN3C(=O)C=CC3=O)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN(C)C(=O)CCCCCN3C(=O)CC(SC[C@H](N)C(=O)O)C3=O)c21]
and pharmaceutically acceptable salts thereof.

118. An antibody-drug conjugate (ADC) having the formula:
Ab-(S*-M¹-(D))ₚ
wherein:
Ab is an antibody;
each S* is a sulfur atom from a cysteine residue of the antibody;
M¹ is a succinimide or a hydrolyzed succinimide;
subscript p is an integer from 2 to 8; and
each (D) is a Drug Unit of Formula (I): 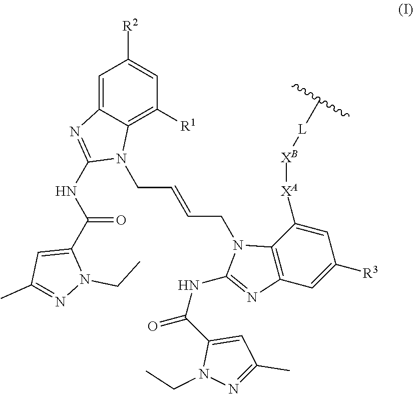
wherein [symbol]
represents covalent attachment of L to M¹;
R¹ is hydrogen, hydroxyl, C₁₋₆ alkoxy, —(C₁₋₆ alkyl)C₁₋₆ alkoxy, —(CH₂)ₙ—NR^{A}R^{B}, or PEG2 to PEG4;
R² and R³ are independently —CO₂H, —(C═O)ₘ—NR^{C}R^{D}, or —(CH₂)_{q}—NR^{E}R^{F};
each R^{A}, R^{B}, R^{C}, R^{D}, R^{E}, and R^{F} are independently hydrogen or C₁₋₃ alkyl;
each subscript n is independently an integer from 0 to 6;
each subscript m is independently 0 or 1;
each subscript q is an integer from 0 to 6;
X^{A} is —CH₂—, —O—, —S—, —NH—, or —N(CH₃)—;
X^{B} is absent or a 2-16 membered heteroalkylene;
L has the formula -(A)ₐ-(W)_{w}-(Y)_{y}—, wherein:
subscript a is 0 or 1;
subscript y is 0 or 1;
subscript w is 0 or 1;
A is a C₂₋₂₀ alkylene optionally substituted with 1-3 R^{a1}; or a 2 to 40 membered heteroalkylene optionally substituted with 1-3 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, =), —NR^{d1}R^{e1}, —C(O)NR^{d1}R^{e1}, —C(O)(C₁₋₆ alkyl), and —C(O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —C(O)NR^{d1}R^{e1}, —C(O)(C₁₋₆ alkyl), and —C(O)O(C₁₋₆ alkyl);
each R^{d1} and R^{e1} are independently hydrogen or C₁₋₃ alkyl;
W is from 1-12 amino acids or has the structure: 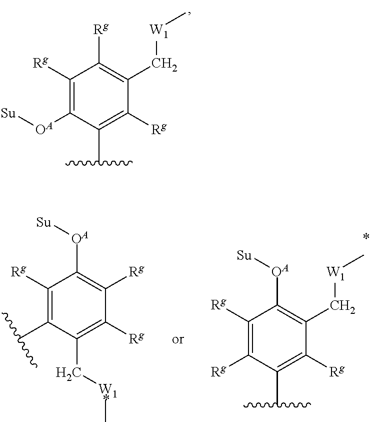
wherein Su is a Sugar moiety;
—O^{A}— represents a glycosidic bond;
each R^{g} is independently hydrogen, halogen, —CN, or —NO₂;
W¹ is absent or —O—C(═O)—; [symbol]
represents covalent attachment to A or M¹;
* represents covalent attachment to Y, X^{B}, or X^{A};
Y is self-immolative moiety, a non-self-immolative releasable moiety, or a non-cleavable moiety; and
X^{B} and L are each independently optionally substituted with a PEG Unit from PEG1 to PEG 72.

119-152. (canceled)

153. The ADC of claim 118, wherein X^{A} is —O—.

154-185. (canceled)

186. The ADC of claim 118, wherein each amino acid in W is selected from the group consisting of alanine, glycine, lysine, serine, aspartic acid, aspartate methyl ester, N,N-dimethyl-lysine, phenylalanine, citrulline, valine-alanine, valine-citrulline, phenylalanine-lysine or homoserine methyl ether.

187-192. (canceled)

193. The ADC of claim 118, wherein A is C₄₋₁₀ alkylene optionally substituted with 1-3 Ra1.

194-208. (canceled)

209. The ADC of claim 118, wherein the linker is a cleavable linker.

210. The ADC of claim 118, wherein the linker is cleavable by one or more of cathepsin B, C, or D; β-glucuronidase; and β-mannosidase.

211. The ADC of claim 118, wherein the linker is a non-cleavable linker.

212-215. (canceled)

216. A compound having the structure of Formula (IV): 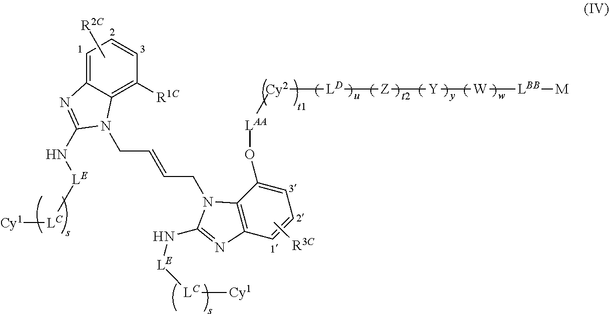
or a pharmaceutically acceptable salt thereof, wherein:
R^{1C} is hydrogen, hydroxyl, C₁₋₆ alkoxy, —(C₁₋₆ alkyl) C₁₋₆ alkoxy, —(CH₂)ₙ—NR^{A}R^{B}, or PEG2 to PEG4;
R^{2C} is —CO₂R^{M}, —(C═O)NR^{C}R^{D}, S(O)₂NR^{C}R^{D}, S(O)₂R^{M}, —(CH₂)_{q}—NR^{E}R^{F}, —(CH₂)_{q}—OR^{M}, —O(C═O)—NR^{E}R^{F}, or —NR^{M}(C═O)—NR^{E}R^{F}, wherein R^{2C} is attached at any one of positions labeled 1, 2, or 3;
R^{3C} is —CO₂R^{M}, —(C═O)NR^{C}R^{D}, —S(O)₂NR^{C}R^{D}, —S(O)₂R^{M}, —(CH₂)_{q}—NR^{E}R^{F}, —(CH₂)_{q}—OR^{M}, —O(C═O)—NR^{E}R^{F}, or —NR^{M}(C═O)—NR^{E}R^{F}, wherein R^{3C} is attached at any one of positions labeled 1′, 2′, or 3′;
each R^{A}, R^{B}, R^{C}, R^{D}, R^{E}, R^{F}, and R^{M} are independently hydrogen or C₁₋₆ alkyl;
each subscript n is independently an integer from 0 to 6;
each subscript q is independently an integer from 0 to 6;
L^{E} is —(C═O)— or —S(O)₂—;
L^{C} is —(CR^{I}R^{J})₁₋₃—
each R^{I} and R^{J} are independently hydrogen or C₁₋₃ alkyl;
subscript s is 0 or 1;
each Cy¹ is independently a 4-6 membered heterocycle, a 5-6 membered heteroaryl, or a C₃₋₆ cycloalkyl, each optionally substituted with one or more R^{K};
each R^{K} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d2}R^{e2}, —C(O)NR^{d2}R^{e2}, —C(O)(C₁₋₆ alkyl), and —C(O)O(C₁₋₆ alkyl);
each R^{d2} and R^{e2} are independently hydrogen or C₁₋₃ alkyl;
L^{AA} is —(CH₂)₁₋₆—, —C(O)(CH₂)₁₋₆—, —C(O)NR^{L}(CH₂)₁₋₆—, —(CH₂)₁₋₆O—, —C(O)(CH₂)₁₋₆O—, or —C(O)NR^{L}(CH₂)₁₋₆O—;
R^{L} is hydrogen or C₁₋₃ alkyl;
Cy² is C₃₋₆ cycloalkyl, 4-6 membered heterocycle, 5-6 membered heteroaryl, or phenyl, each optionally substituted with one or more R^{U};
each R^{U} is independently selected from the group consisting of —CO₂R^{j1}, —(C═O)NR^{d3}R^{e3}, —S(O)₂NR^{d3}R^{e3}, —(CH₂)_{q1}—NR^{g1}R^{h1}, —(CH₂)_{q1}—OR^{j1}, and —(CH₂)_{q1}—(OCH₂CH₂)₁₋₈OH;
each R^{d3}, R^{e3}, R^{g1}, R^{h1}, and R^{j1} are independently hydrogen or C₁₋₆ alkyl;
subscript q1 is an integer from 0 to 6;
subscripts t1 and t2 are independently 0 or 1, wherein at least one of t1 and t2 is 1;
L^{D} is —(CH₂)₁₋₆—;
subscript u is 0 or 1;
Z is —N(R^{HH})— or —N⁺(C₁₋₆ alkyl)(R^{HH})—;
R^{HH} is hydrogen, C₁₋₆ alkyl, C₃₋₆ cycloalkyl, —(CH₂)₁₋₃C₃₋₆ cycloalkyl, —(CH₂)₁₋₃C₁₋₃ alkoxy, —(CH₂)₁₋₃ 4-6 membered heterocycle, or —(CH₂)₁₋₃ 5-6 membered heteroaryl;
Y is a self-immolative moiety, a non-self-immolative releasable moiety, or a non-cleavable moiety;
subscript y is 0 or 1;
W is a chain of 1-12 amino acids or has the structure: 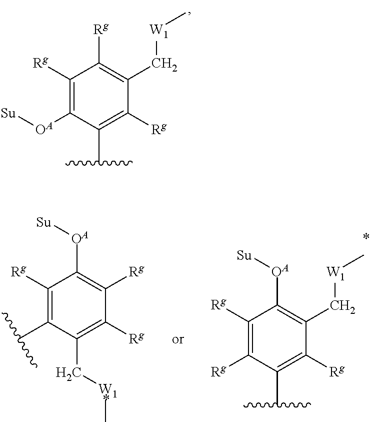
wherein Su is a Sugar moiety;
—O^{A}— represents a glycosidic bond;
each R^{g} is independently hydrogen, halogen, —CN, or —NO₂;
W¹ is absent or —O—C(═O)—; [symbol]
represents covalent attachment to L^{BB};
* represents covalent attachment to Y, L^{D}, NR^{HH}, or Cy²;
subscript w is 0 or 1;
L^{BB} is —(CH₂)₁₋₆—, —C(O)(CH₂)₁₋₆—, or —[NHC(O)(CH₂)₁₋₄]₁₋₃—; and
M is 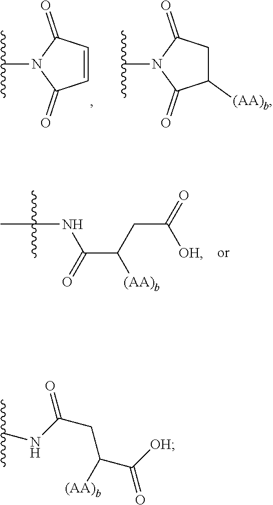
each AA is an independently selected amino acid, wherein (AA)_{b} is connected to the succinimide or hydrolyzed succinimide via a sulfur atom; and
each subscript b is independently an integer from 1 to 6.

217-302. (canceled)

303. The compound of claim 216, wherein Cy² is a 4-6 membered heterocycle.

304-331. (canceled)

332. The compound of claim 216, wherein t1 is 0 and t2 is 1.

333. The compound of claim 216, wherein t1 is 1 and t2 is 0.

334-337. (canceled)

338. The compound of claim 216, wherein t2 is 1 and R^{HH} is C₁₋₃ alkyl.

339-351. (canceled)

352. The compound of claim 216, wherein each amino acid of W is independently selected from the group consisting of alanine, valine, isoleucine, leucine, aspartic acid, glutamic acid, lysine, histidine, arginine, glycine, serine, threonine, phenylalanine, O-methylserine, O-methylaspartic acid, O-methylglutamic acid, N-methyllysine, O-methyltyrosine, O-methylhistidine, and O-methylthreonine.

353-359. (canceled)

360. The compound of claim 216, wherein L^{BB} is —(CH₂)₁₋₃—.

361-364. (canceled)

365. The compound of claim 216, wherein M is

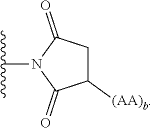

366-376. (canceled)

377. The compound of claim 216, selected from the group consisting of 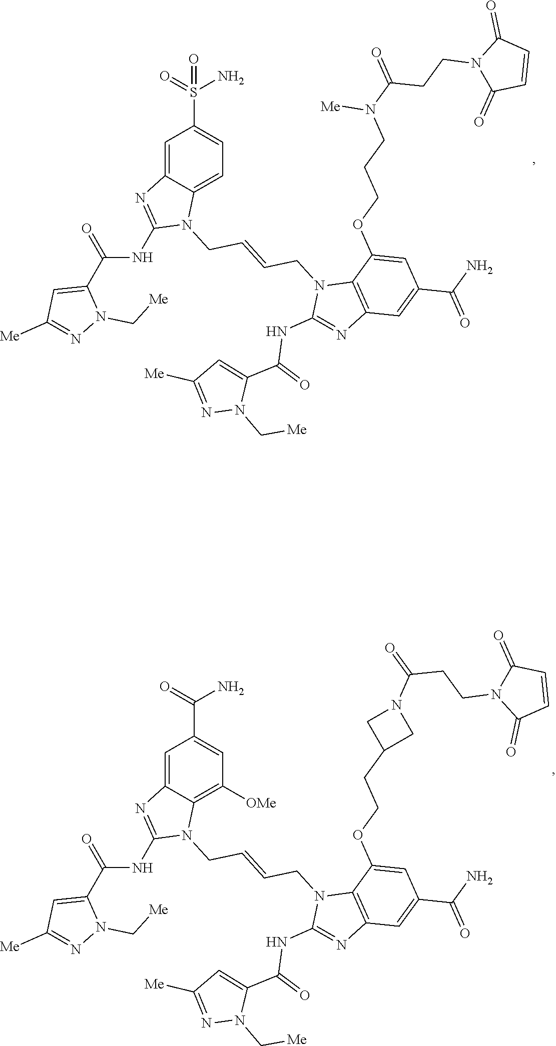 [SMILES: CCn1nc(C)cc1C(=O)Nc1nc2cc(S(N)(=O)=O)ccc2n1C/C=C/Cn1c(NC(=O)c2cc(C)nn2CC)nc2cc(C(N)=O)cc(OCCCN(C)C(=O)CCN3C(=O)C=CC3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCC3CN(C(=O)CCN4C(=O)C=CC4=O)C3)c21] 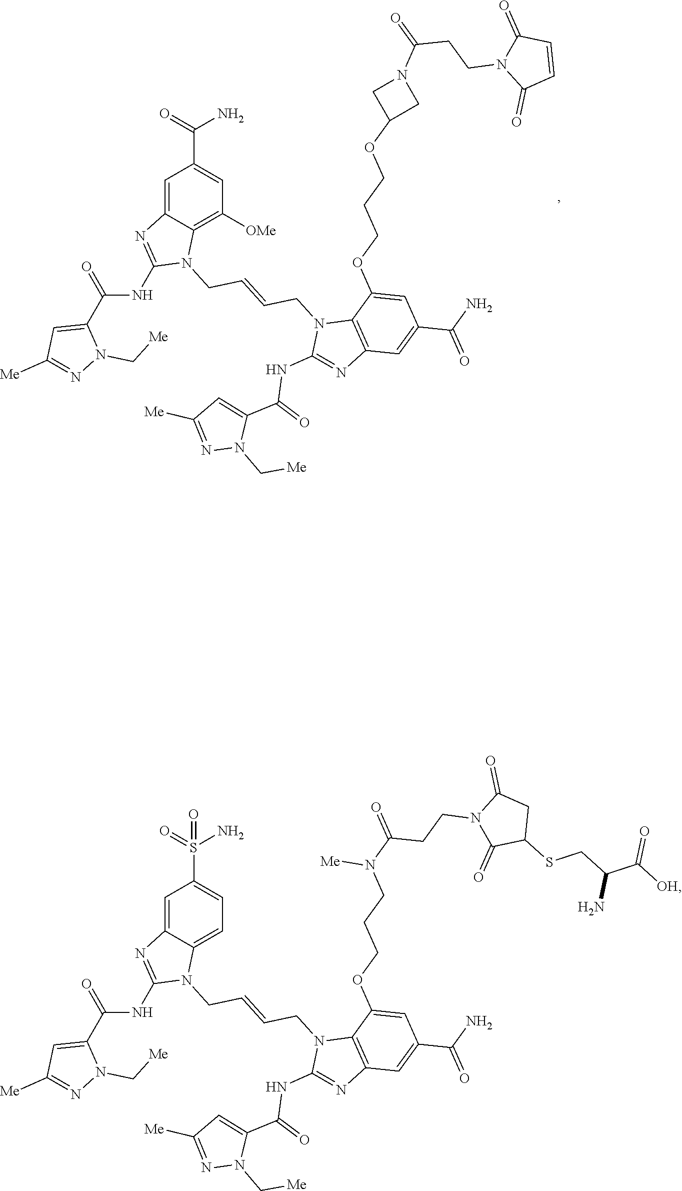 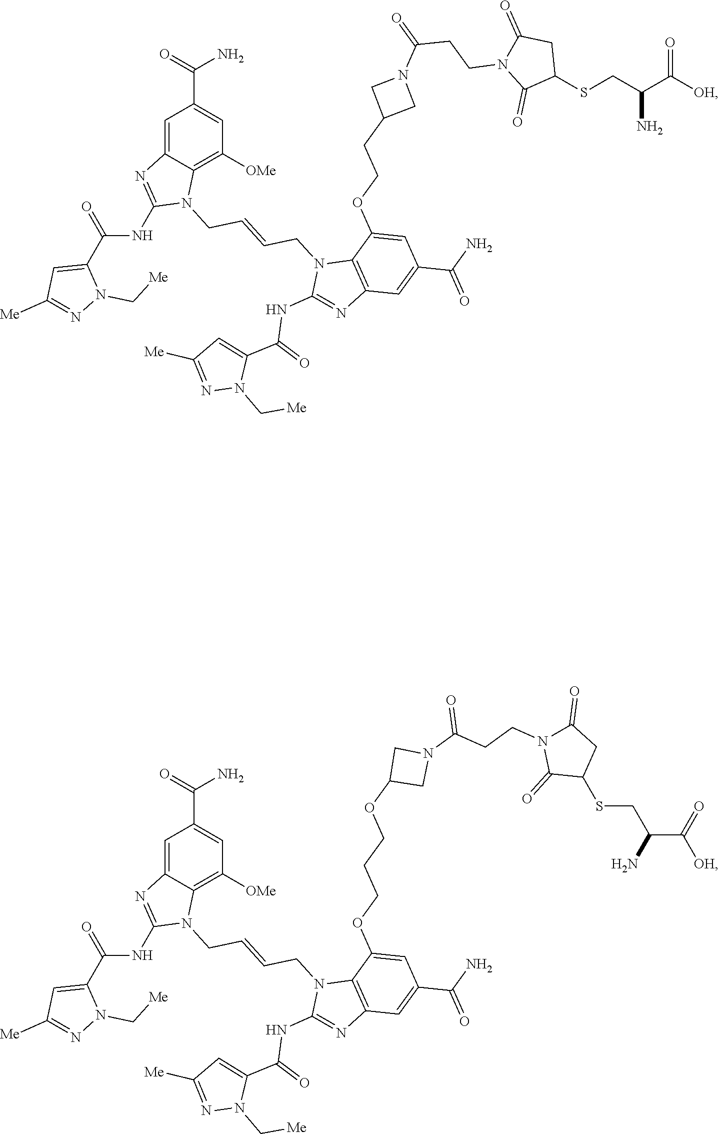 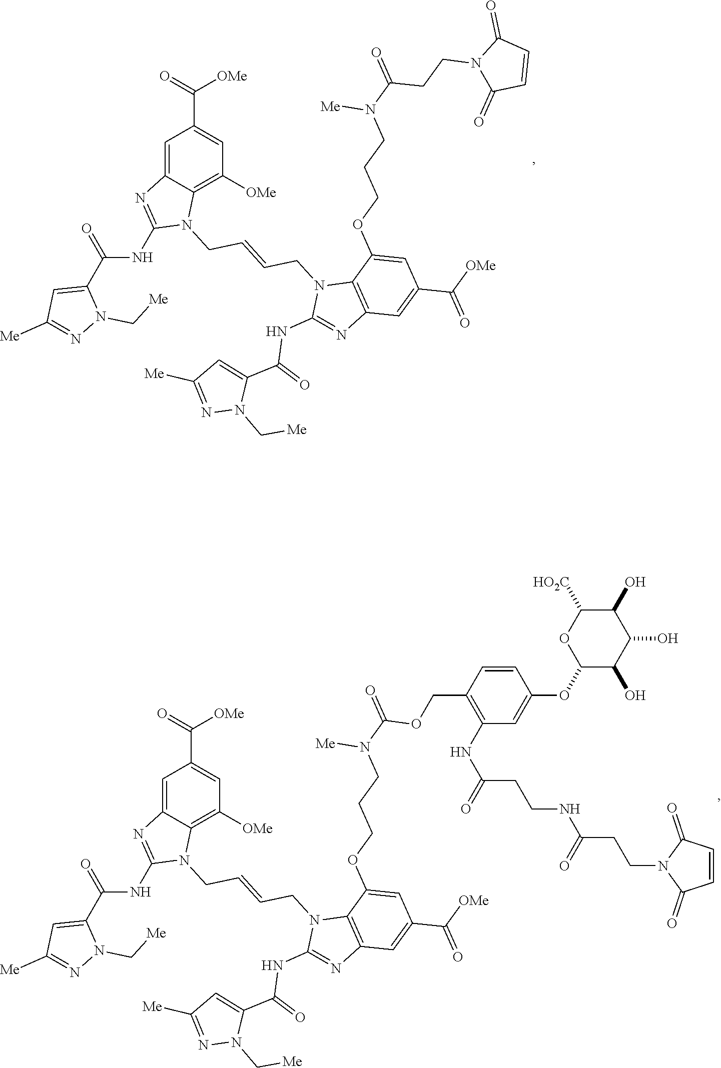 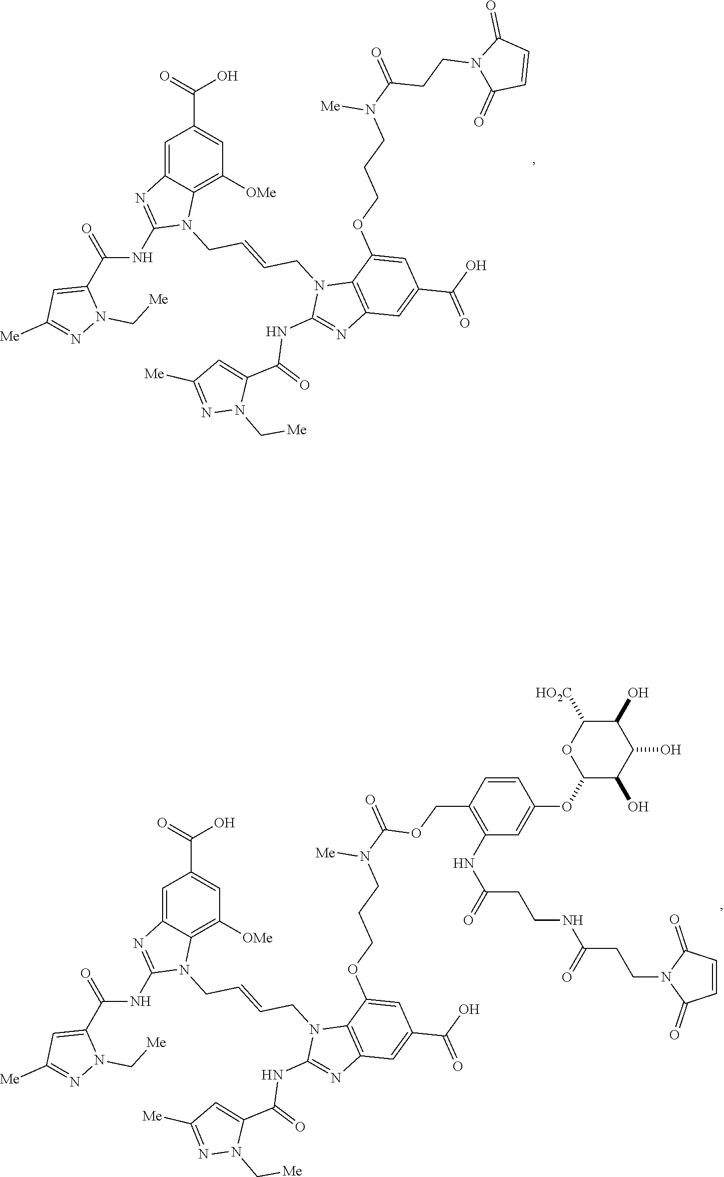 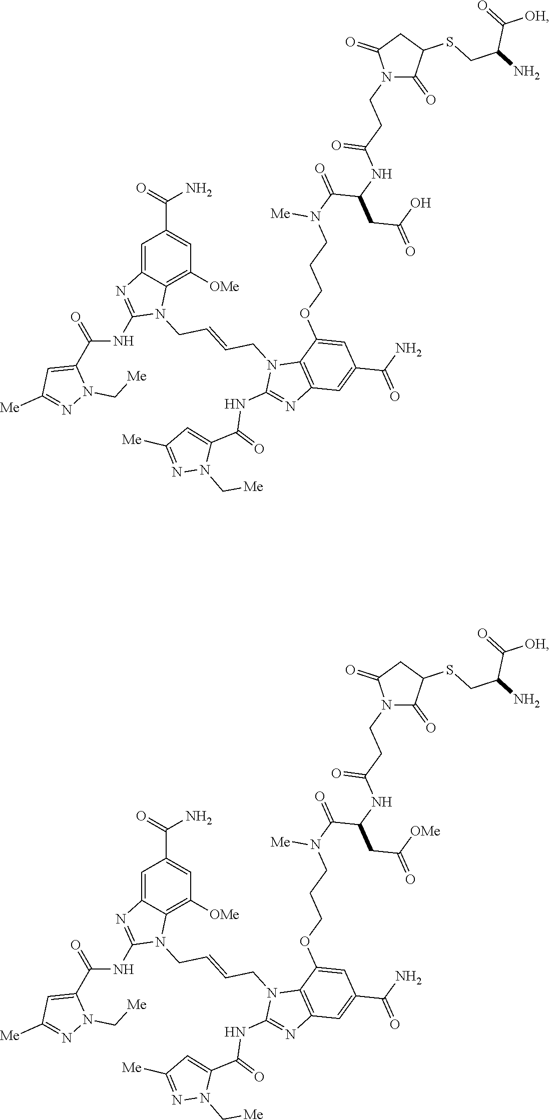 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CC(=O)O)NC(=O)CCN3C(=O)CC(SC[C@H](N)C(=O)O)C3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CC(=O)OC)NC(=O)CCN3C(=O)CC(SC[C@H](N)C(=O)O)C3=O)c21] 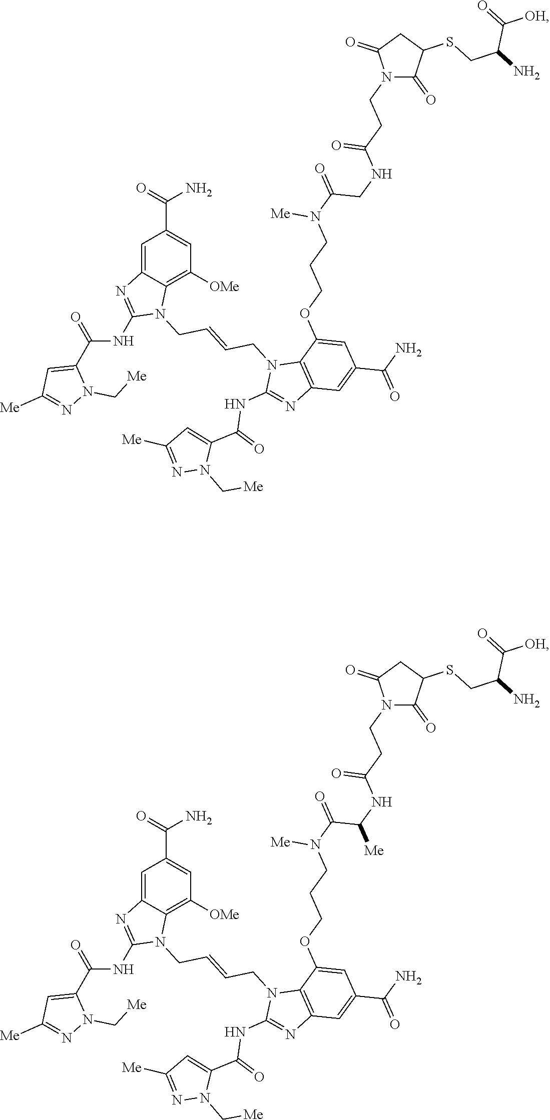 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)CNC(=O)CCN3C(=O)CC(SC[C@H](N)C(=O)O)C3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](C)NC(=O)CCN3C(=O)CC(SC[C@H](N)C(=O)O)C3=O)c21] 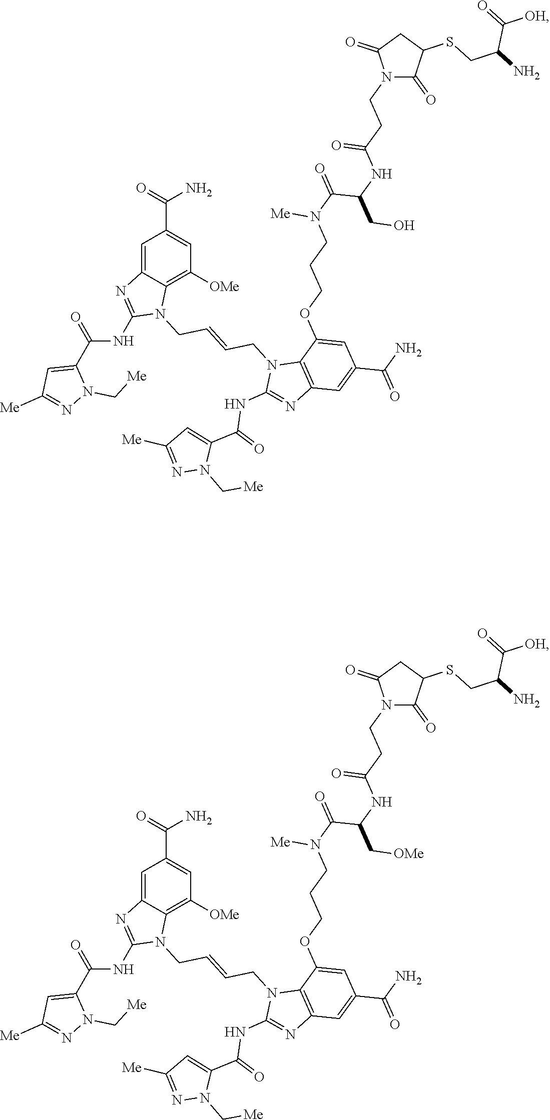 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](CO)NC(=O)CCN3C(=O)CC(SC[C@H](N)C(=O)O)C3=O)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)C(=O)[C@H](COC)NC(=O)CCN3C(=O)CC(SC[C@H](N)C(=O)O)C3=O)c21] 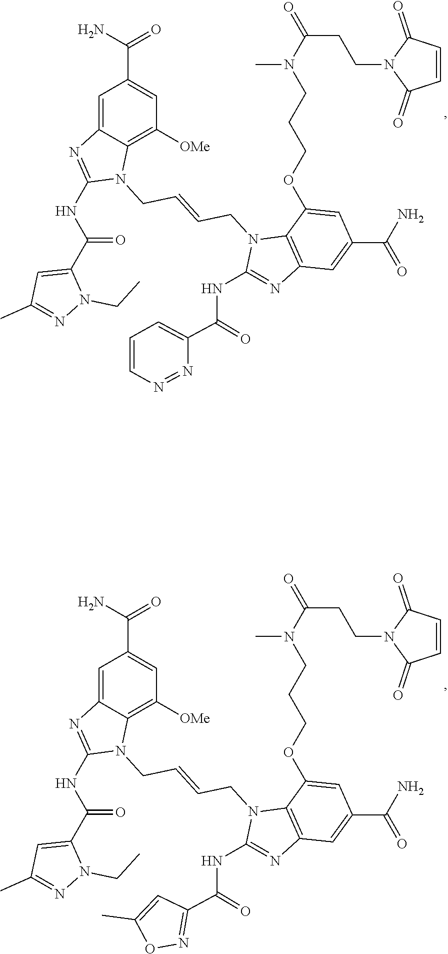 [SMILES: CCn1nc(C)cc1C(=O)Nc1nc2cc(C(N)=O)cc(OC)c2n1C/C=C/Cn1c(NC(=O)c2cccnn2)nc2cc(C(N)=O)cc(OCCCN(C)C(=O)CCN3C(=O)C=CC3=O)c21 ; CCn1nc(C)cc1C(=O)Nc1nc2cc(C(N)=O)cc(OC)c2n1C/C=C/Cn1c(NC(=O)c2cc(C)on2)nc2cc(C(N)=O)cc(OCCCN(C)C(=O)CCN3C(=O)C=CC3=O)c21] 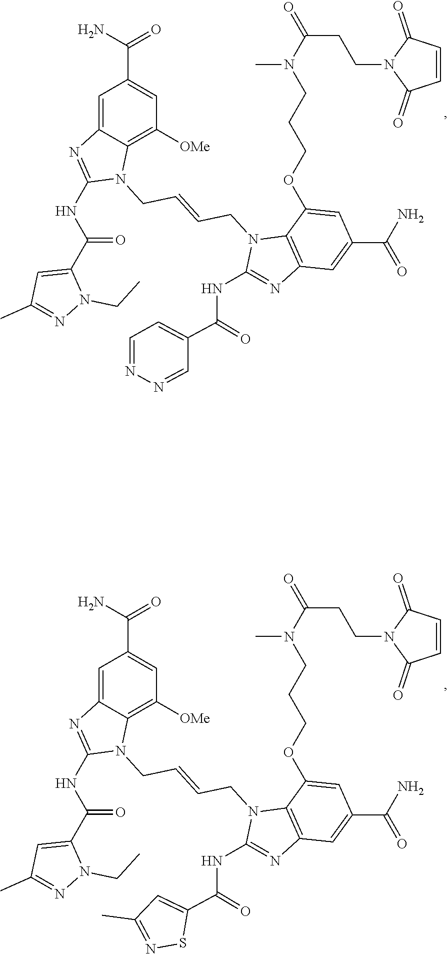 [SMILES: CCn1nc(C)cc1C(=O)Nc1nc2cc(C(N)=O)cc(OC)c2n1C/C=C/Cn1c(NC(=O)c2ccnnc2)nc2cc(C(N)=O)cc(OCCCN(C)C(=O)CCN3C(=O)C=CC3=O)c21 ; CCn1nc(C)cc1C(=O)Nc1nc2cc(C(N)=O)cc(OC)c2n1C/C=C/Cn1c(NC(=O)c2cc(C)ns2)nc2cc(C(N)=O)cc(OCCCN(C)C(=O)CCN3C(=O)C=CC3=O)c21] 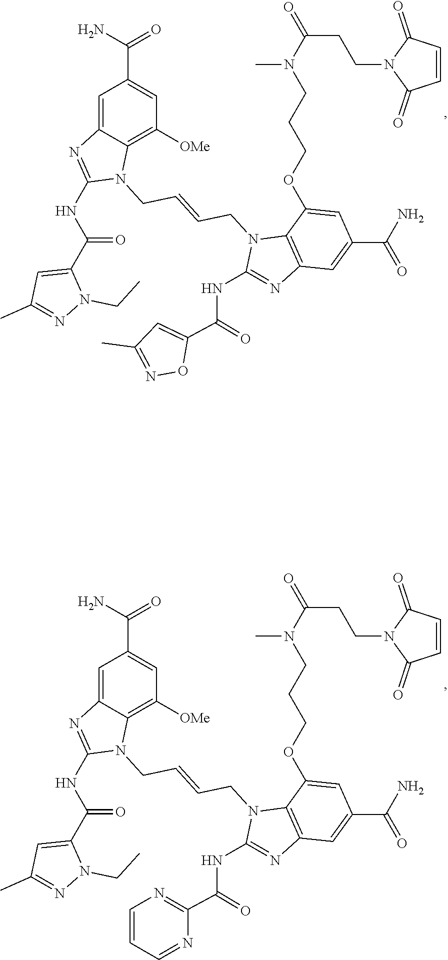 [SMILES: CCn1nc(C)cc1C(=O)Nc1nc2cc(C(N)=O)cc(OC)c2n1C/C=C/Cn1c(NC(=O)c2cc(C)no2)nc2cc(C(N)=O)cc(OCCCN(C)C(=O)CCN3C(=O)C=CC3=O)c21 ; CCn1nc(C)cc1C(=O)Nc1nc2cc(C(N)=O)cc(OC)c2n1C/C=C/Cn1c(NC(=O)c2ncccn2)nc2cc(C(N)=O)cc(OCCCN(C)C(=O)CCN3C(=O)C=CC3=O)c21] 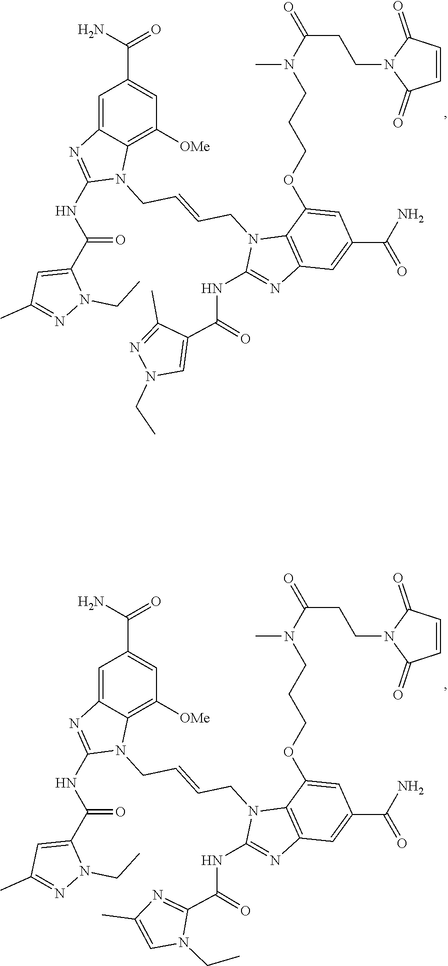 [SMILES: CCn1cc(C(=O)Nc2nc3cc(C(N)=O)cc(OCCCN(C)C(=O)CCN4C(=O)C=CC4=O)c3n2C/C=C/Cn2c(NC(=O)c3cc(C)nn3CC)nc3cc(C(N)=O)cc(OC)c32)c(C)n1 ; CCn1cc(C)nc1C(=O)Nc1nc2cc(C(N)=O)cc(OCCCN(C)C(=O)CCN3C(=O)C=CC3=O)c2n1C/C=C/Cn1c(NC(=O)c2cc(C)nn2CC)nc2cc(C(N)=O)cc(OC)c21] 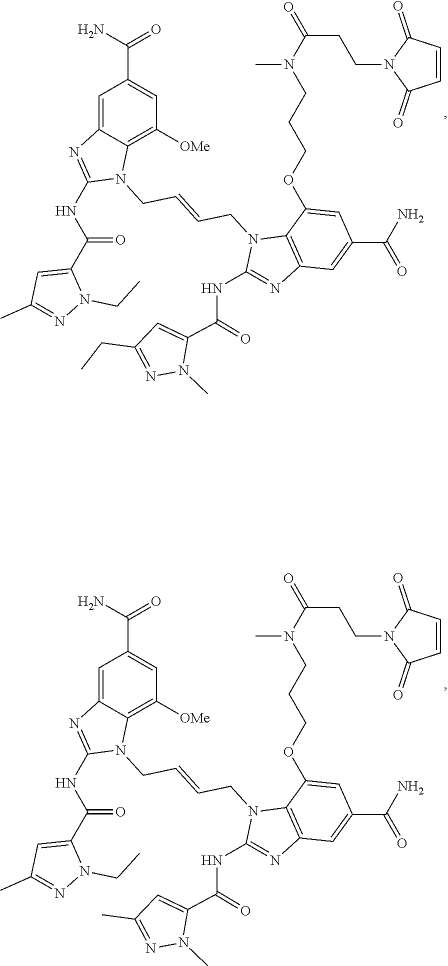 [SMILES: CCc1cc(C(=O)Nc2nc3cc(C(N)=O)cc(OCCCN(C)C(=O)CCN4C(=O)C=CC4=O)c3n2C/C=C/Cn2c(NC(=O)c3cc(C)nn3CC)nc3cc(C(N)=O)cc(OC)c32)n(C)n1 ; CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCCN(C)C(=O)CCN3C(=O)C=CC3=O)c21] 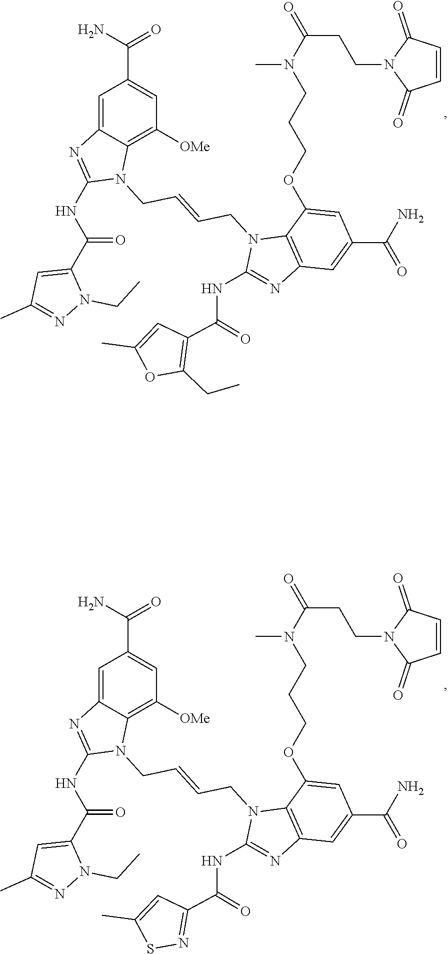 [SMILES: CCc1oc(C)cc1C(=O)Nc1nc2cc(C(N)=O)cc(OCCCN(C)C(=O)CCN3C(=O)C=CC3=O)c2n1C/C=C/Cn1c(NC(=O)c2cc(C)nn2CC)nc2cc(C(N)=O)cc(OC)c21 ; CCn1nc(C)cc1C(=O)Nc1nc2cc(C(N)=O)cc(OC)c2n1C/C=C/Cn1c(NC(=O)c2cc(C)sn2)nc2cc(C(N)=O)cc(OCCCN(C)C(=O)CCN3C(=O)C=CC3=O)c21] 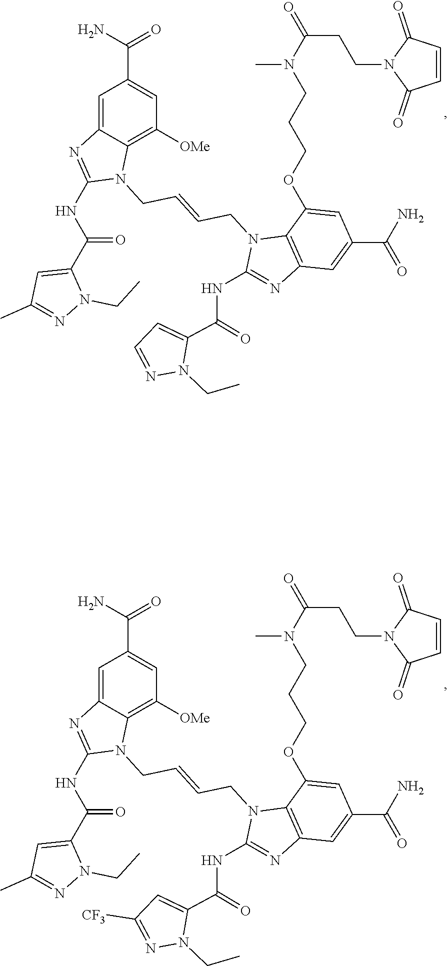 [SMILES: CCn1nc(C)cc1C(=O)Nc1nc2cc(C(N)=O)cc(OC)c2n1C/C=C/Cn1c(NC(=O)c2ccnn2CC)nc2cc(C(N)=O)cc(OCCCN(C)C(=O)CCN3C(=O)C=CC3=O)c21 ; CCn1nc(C)cc1C(=O)Nc1nc2cc(C(N)=O)cc(OC)c2n1C/C=C/Cn1c(NC(=O)c2cc(C(F)(F)F)nn2CC)nc2cc(C(N)=O)cc(OCCCN(C)C(=O)CCN3C(=O)C=CC3=O)c21] 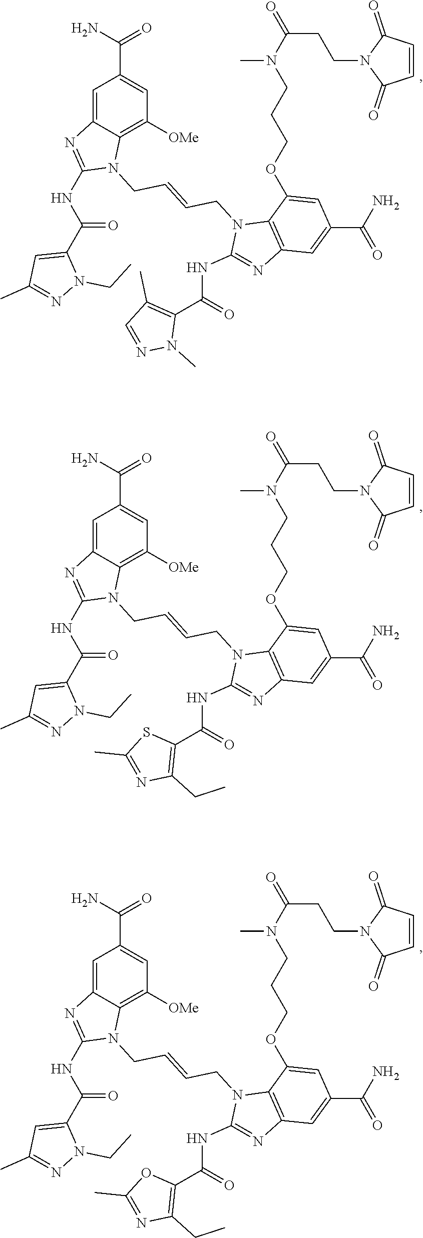 [SMILES: CCn1nc(C)cc1C(=O)Nc1nc2cc(C(N)=O)cc(OC)c2n1C/C=C/Cn1c(NC(=O)c2c(C)cnn2C)nc2cc(C(N)=O)cc(OCCCN(C)C(=O)CCN3C(=O)C=CC3=O)c21 ; CCc1nc(C)sc1C(=O)Nc1nc2cc(C(N)=O)cc(OCCCN(C)C(=O)CCN3C(=O)C=CC3=O)c2n1C/C=C/Cn1c(NC(=O)c2cc(C)nn2CC)nc2cc(C(N)=O)cc(OC)c21 ; CCc1nc(C)oc1C(=O)Nc1nc2cc(C(N)=O)cc(OCCCN(C)C(=O)CCN3C(=O)C=CC3=O)c2n1C/C=C/Cn1c(NC(=O)c2cc(C)nn2CC)nc2cc(C(N)=O)cc(OC)c21] 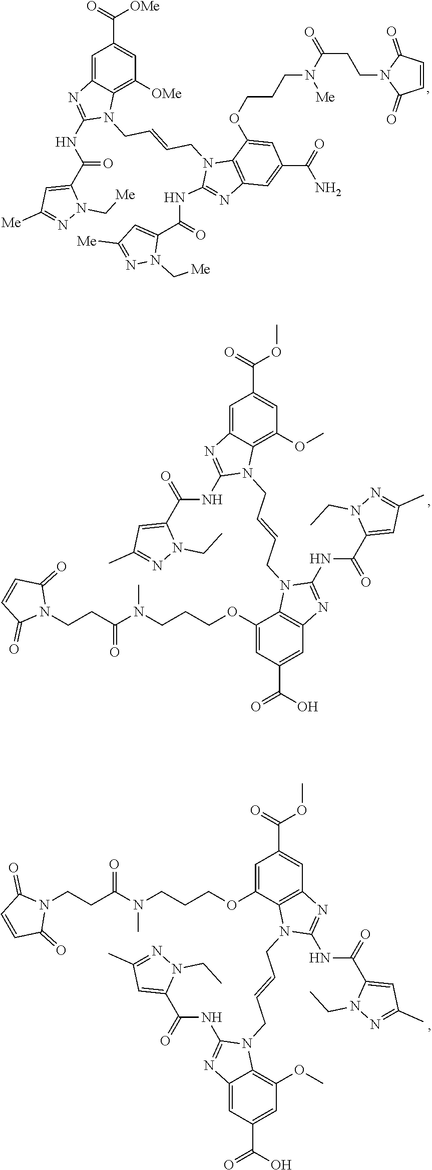 [SMILES: CCn1nc(C)cc1C(=O)Nc1nc2cc(C(=O)OC)cc(OC)c2n1C/C=C/Cn1c(NC(=O)c2cc(C)nn2CC)nc2cc(C(N)=O)cc(OCCCN(C)C(=O)CCN3C(=O)C=CC3=O)c21 ; CCn1nc(C)cc1C(=O)Nc1nc2cc(C(=O)OC)cc(OC)c2n1C/C=C/Cn1c(NC(=O)c2cc(C)nn2CC)nc2cc(C(=O)O)cc(OCCCN(C)C(=O)CCN3C(=O)C=CC3=O)c21 ; CCn1nc(C)cc1C(=O)Nc1nc2cc(C(=O)O)cc(OC)c2n1C/C=C/Cn1c(NC(=O)c2cc(C)nn2CC)nc2cc(C(=O)OC)cc(OCCCN(C)C(=O)CCN3C(=O)C=CC3=O)c21] 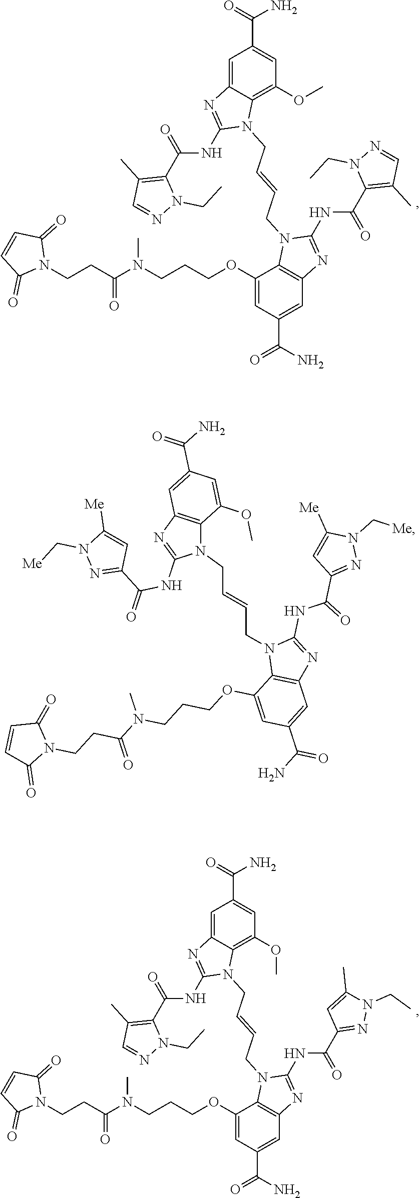 [SMILES: CCn1ncc(C)c1C(=O)Nc1nc2cc(C(N)=O)cc(OC)c2n1C/C=C/Cn1c(NC(=O)c2c(C)cnn2CC)nc2cc(C(N)=O)cc(OCCCN(C)C(=O)CCN3C(=O)C=CC3=O)c21 ; CCn1nc(C(=O)Nc2nc3cc(C(N)=O)cc(OC)c3n2C/C=C/Cn2c(NC(=O)c3cc(C)n(CC)n3)nc3cc(C(N)=O)cc(OCCCN(C)C(=O)CCN4C(=O)C=CC4=O)c32)cc1C ; CCn1nc(C(=O)Nc2nc3cc(C(N)=O)cc(OCCCN(C)C(=O)CCN4C(=O)C=CC4=O)c3n2C/C=C/Cn2c(NC(=O)c3c(C)cnn3CC)nc3cc(C(N)=O)cc(OC)c32)cc1C] 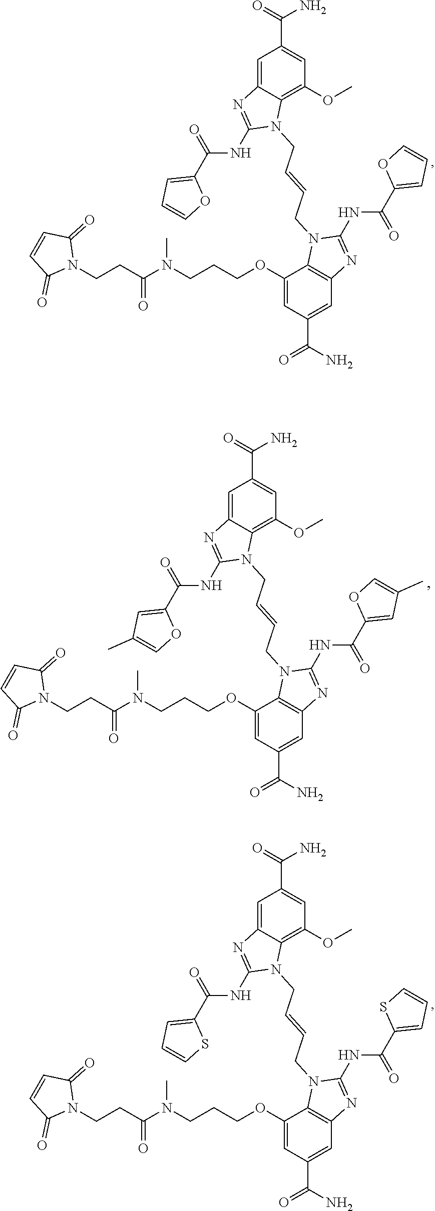 [SMILES: COc1cc(C(N)=O)cc2nc(NC(=O)c3ccco3)n(C/C=C/Cn3c(NC(=O)c4ccco4)nc4cc(C(N)=O)cc(OCCCN(C)C(=O)CCN5C(=O)C=CC5=O)c43)c12 ; COc1cc(C(N)=O)cc2nc(NC(=O)c3cc(C)co3)n(C/C=C/Cn3c(NC(=O)c4cc(C)co4)nc4cc(C(N)=O)cc(OCCCN(C)C(=O)CCN5C(=O)C=CC5=O)c43)c12 ; COc1cc(C(N)=O)cc2nc(NC(=O)c3cccs3)n(C/C=C/Cn3c(NC(=O)c4cccs4)nc4cc(C(N)=O)cc(OCCCN(C)C(=O)CCN5C(=O)C=CC5=O)c43)c12] 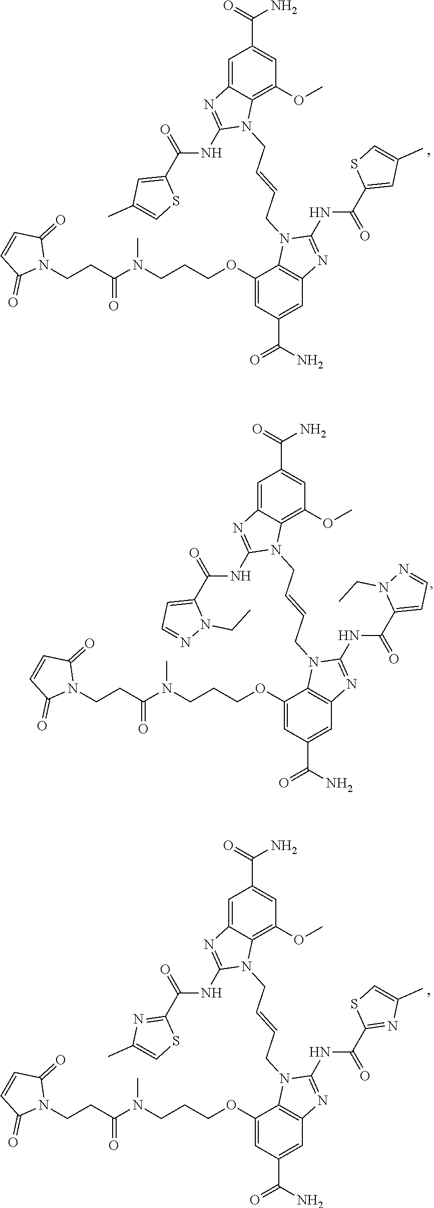 [SMILES: COc1cc(C(N)=O)cc2nc(NC(=O)c3cc(C)cs3)n(C/C=C/Cn3c(NC(=O)c4cc(C)cs4)nc4cc(C(N)=O)cc(OCCCN(C)C(=O)CCN5C(=O)C=CC5=O)c43)c12 ; CCn1nccc1C(=O)Nc1nc2cc(C(N)=O)cc(OC)c2n1C/C=C/Cn1c(NC(=O)c2ccnn2CC)nc2cc(C(N)=O)cc(OCCCN(C)C(=O)CCN3C(=O)C=CC3=O)c21 ; COc1cc(C(N)=O)cc2nc(NC(=O)c3nc(C)cs3)n(C/C=C/Cn3c(NC(=O)c4nc(C)cs4)nc4cc(C(N)=O)cc(OCCCN(C)C(=O)CCN5C(=O)C=CC5=O)c43)c12] 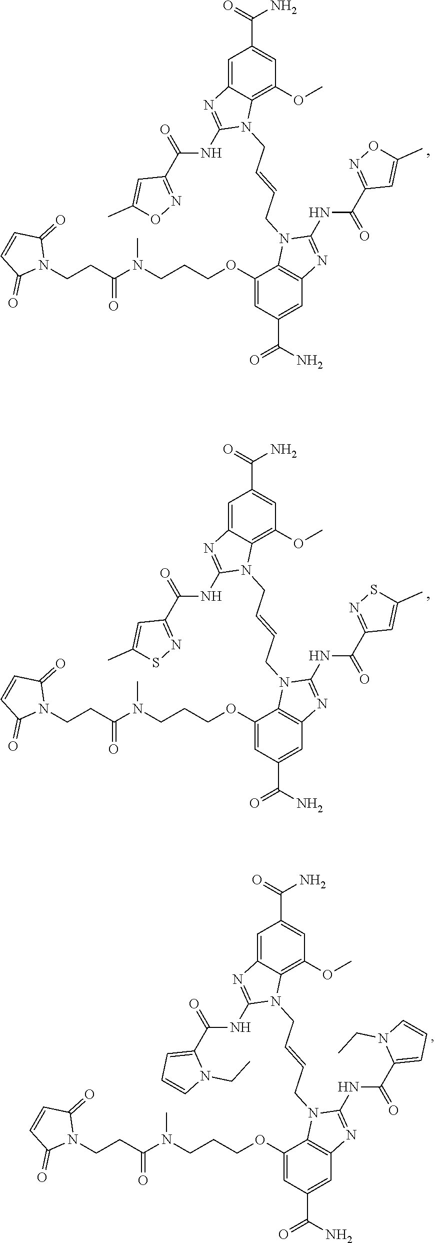 [SMILES: COc1cc(C(N)=O)cc2nc(NC(=O)c3cc(C)on3)n(C/C=C/Cn3c(NC(=O)c4cc(C)on4)nc4cc(C(N)=O)cc(OCCCN(C)C(=O)CCN5C(=O)C=CC5=O)c43)c12 ; COc1cc(C(N)=O)cc2nc(NC(=O)c3cc(C)sn3)n(C/C=C/Cn3c(NC(=O)c4cc(C)sn4)nc4cc(C(N)=O)cc(OCCCN(C)C(=O)CCN5C(=O)C=CC5=O)c43)c12 ; CCn1cccc1C(=O)Nc1nc2cc(C(N)=O)cc(OC)c2n1C/C=C/Cn1c(NC(=O)c2cccn2CC)nc2cc(C(N)=O)cc(OCCCN(C)C(=O)CCN3C(=O)C=CC3=O)c21] 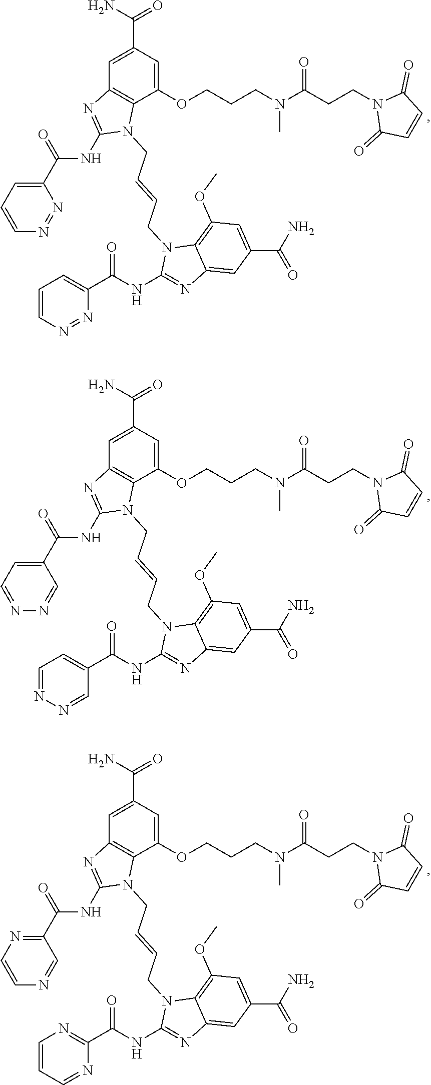 [SMILES: COc1cc(C(N)=O)cc2nc(NC(=O)c3cccnn3)n(C/C=C/Cn3c(NC(=O)c4cccnn4)nc4cc(C(N)=O)cc(OCCCN(C)C(=O)CCN5C(=O)C=CC5=O)c43)c12 ; COc1cc(C(N)=O)cc2nc(NC(=O)c3ccnnc3)n(C/C=C/Cn3c(NC(=O)c4ccnnc4)nc4cc(C(N)=O)cc(OCCCN(C)C(=O)CCN5C(=O)C=CC5=O)c43)c12 ; COc1cc(C(N)=O)cc2nc(NC(=O)c3ncccn3)n(C/C=C/Cn3c(NC(=O)c4cnccn4)nc4cc(C(N)=O)cc(OCCCN(C)C(=O)CCN5C(=O)C=CC5=O)c43)c12] 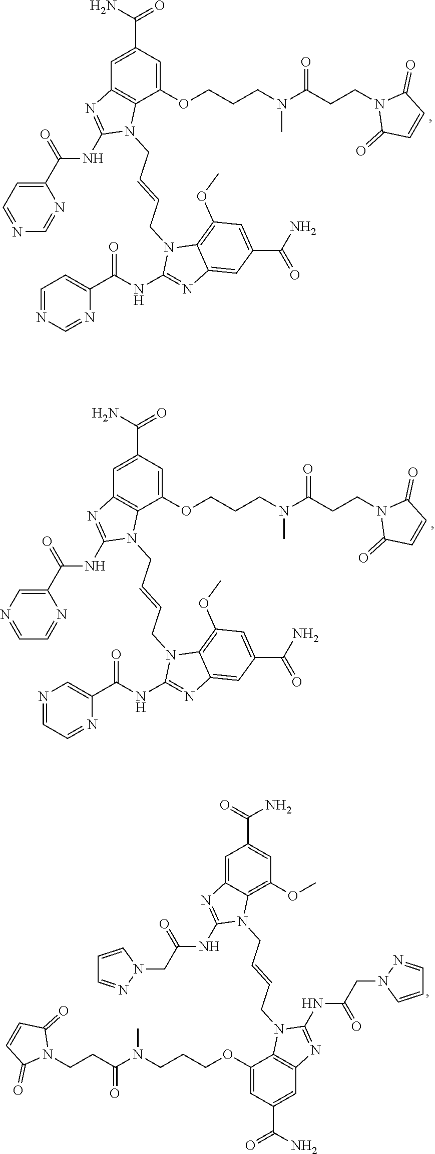 [SMILES: COc1cc(C(N)=O)cc2nc(NC(=O)c3ccncn3)n(C/C=C/Cn3c(NC(=O)c4ccncn4)nc4cc(C(N)=O)cc(OCCCN(C)C(=O)CCN5C(=O)C=CC5=O)c43)c12 ; COc1cc(C(N)=O)cc2nc(NC(=O)c3cnccn3)n(C/C=C/Cn3c(NC(=O)c4cnccn4)nc4cc(C(N)=O)cc(OCCCN(C)C(=O)CCN5C(=O)C=CC5=O)c43)c12 ; COc1cc(C(N)=O)cc2nc(NC(=O)Cn3cccn3)n(C/C=C/Cn3c(NC(=O)Cn4cccn4)nc4cc(C(N)=O)cc(OCCCN(C)C(=O)CCN5C(=O)C=CC5=O)c43)c12] 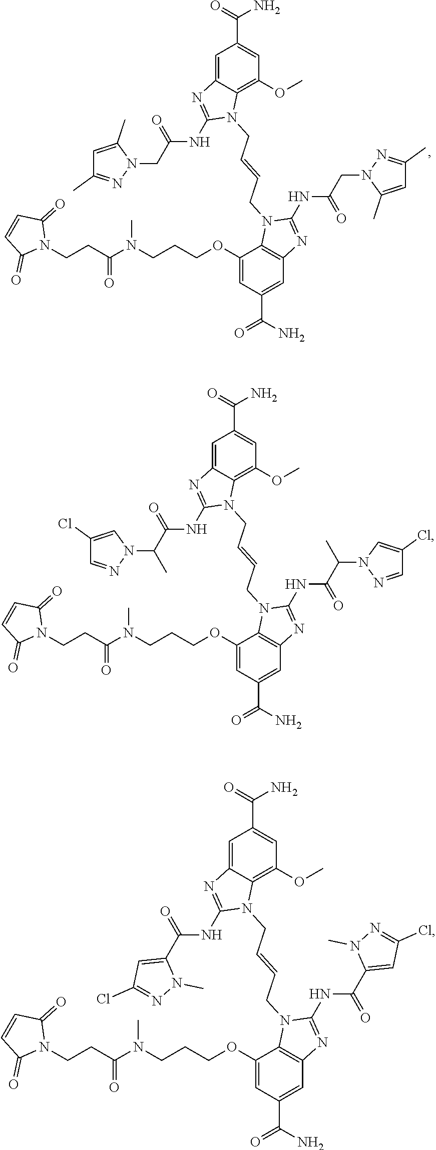 [SMILES: COc1cc(C(N)=O)cc2nc(NC(=O)Cn3nc(C)cc3C)n(C/C=C/Cn3c(NC(=O)Cn4nc(C)cc4C)nc4cc(C(N)=O)cc(OCCCN(C)C(=O)CCN5C(=O)C=CC5=O)c43)c12 ; COc1cc(C(N)=O)cc2nc(NC(=O)C(C)n3cc(Cl)cn3)n(C/C=C/Cn3c(NC(=O)C(C)n4cc(Cl)cn4)nc4cc(C(N)=O)cc(OCCCN(C)C(=O)CCN5C(=O)C=CC5=O)c43)c12 ; COc1cc(C(N)=O)cc2nc(NC(=O)c3cc(Cl)nn3C)n(C/C=C/Cn3c(NC(=O)c4cc(Cl)nn4C)nc4cc(C(N)=O)cc(OCCCN(C)C(=O)CCN5C(=O)C=CC5=O)c43)c12] 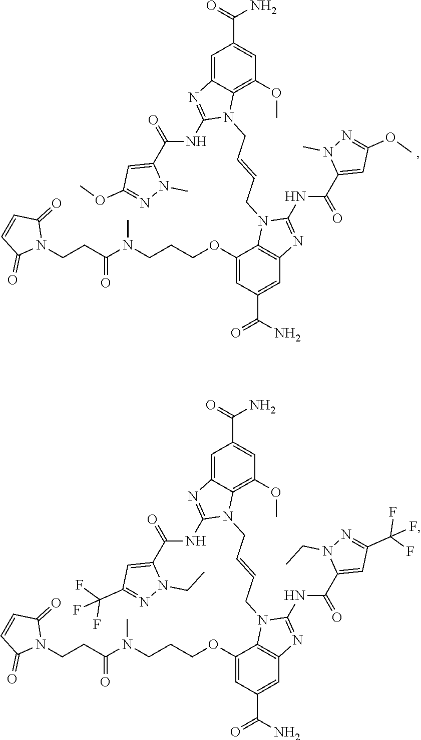 [SMILES: COc1cc(C(=O)Nc2nc3cc(C(N)=O)cc(OC)c3n2C/C=C/Cn2c(NC(=O)c3cc(OC)nn3C)nc3cc(C(N)=O)cc(OCCCN(C)C(=O)CCN4C(=O)C=CC4=O)c32)n(C)n1 ; CCn1nc(C(F)(F)F)cc1C(=O)Nc1nc2cc(C(N)=O)cc(OC)c2n1C/C=C/Cn1c(NC(=O)c2cc(C(F)(F)F)nn2CC)nc2cc(C(N)=O)cc(OCCCN(C)C(=O)CCN3C(=O)C=CC3=O)c21] 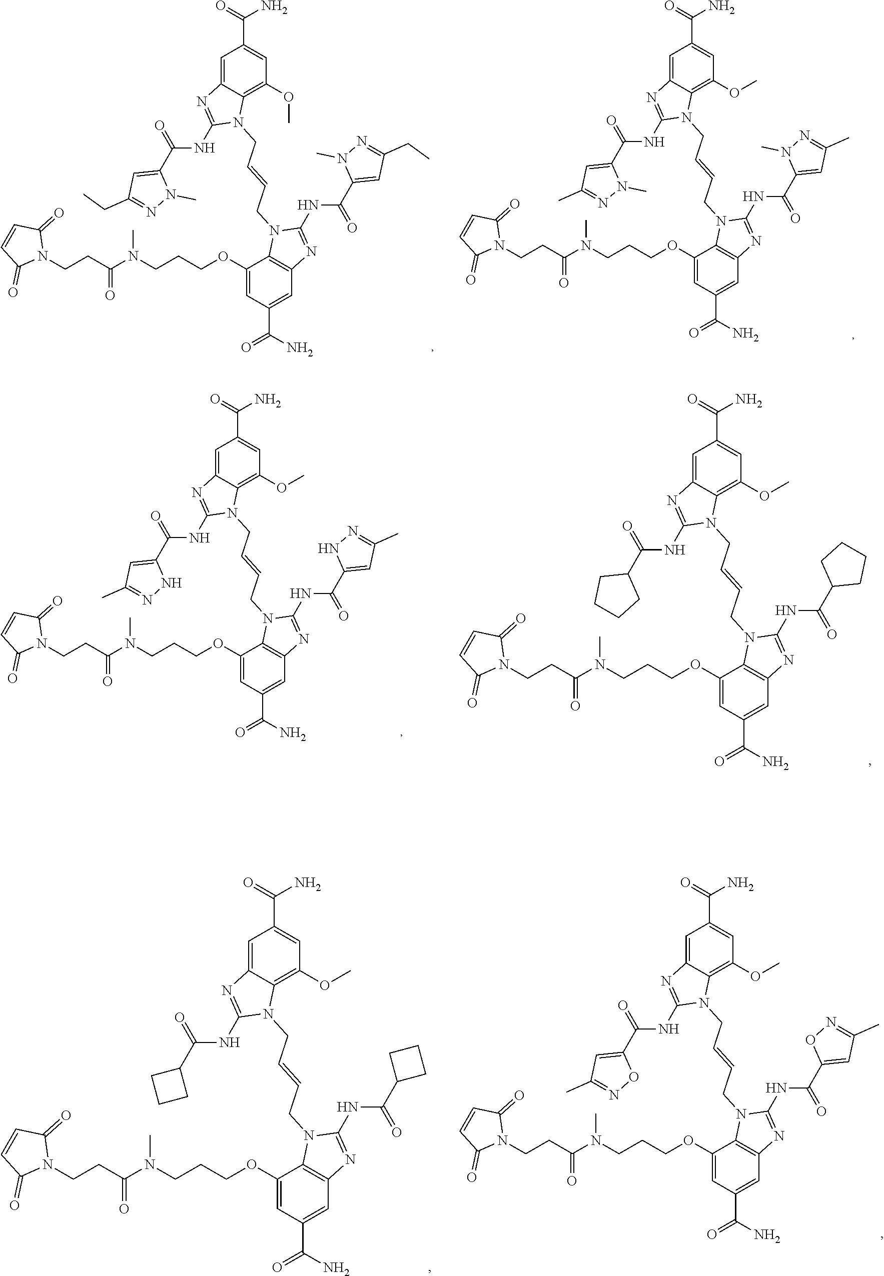 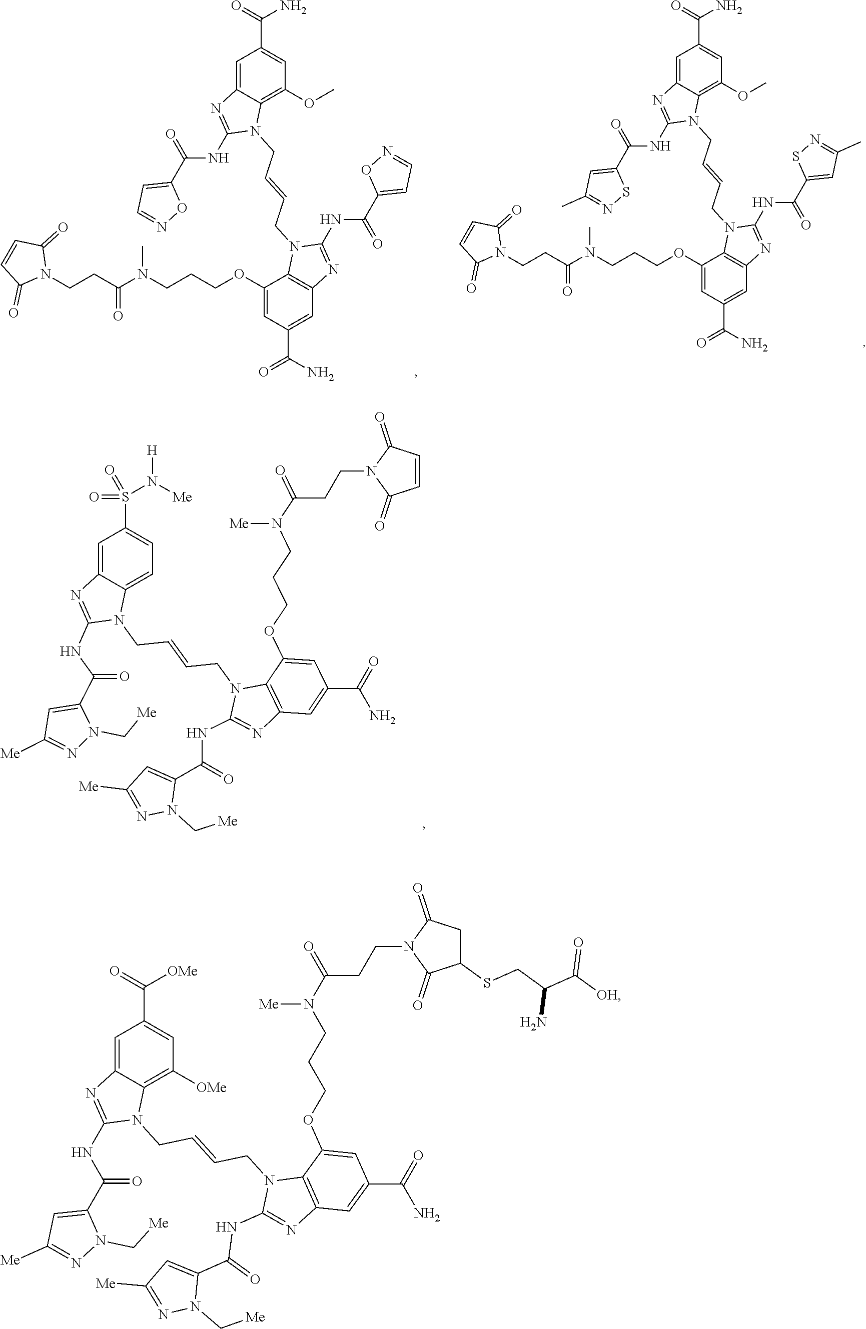 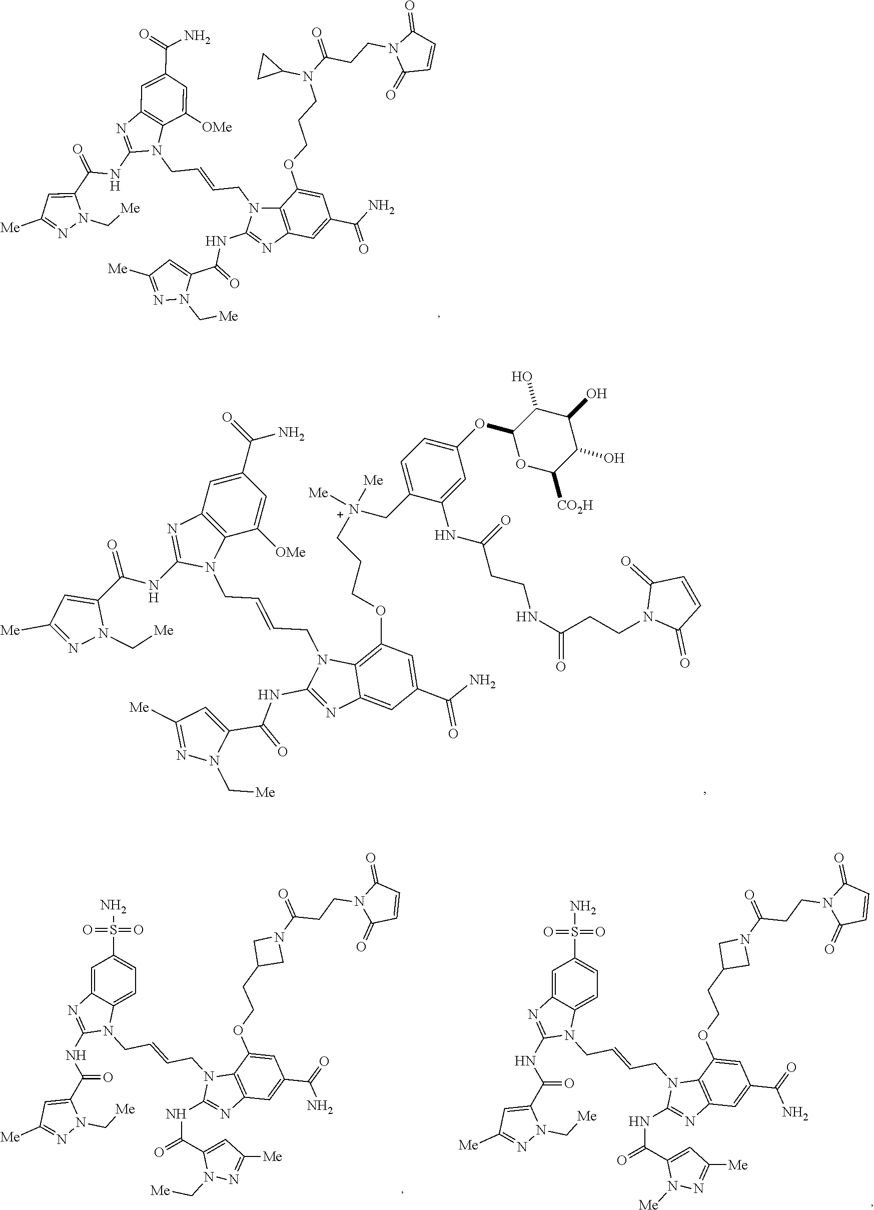 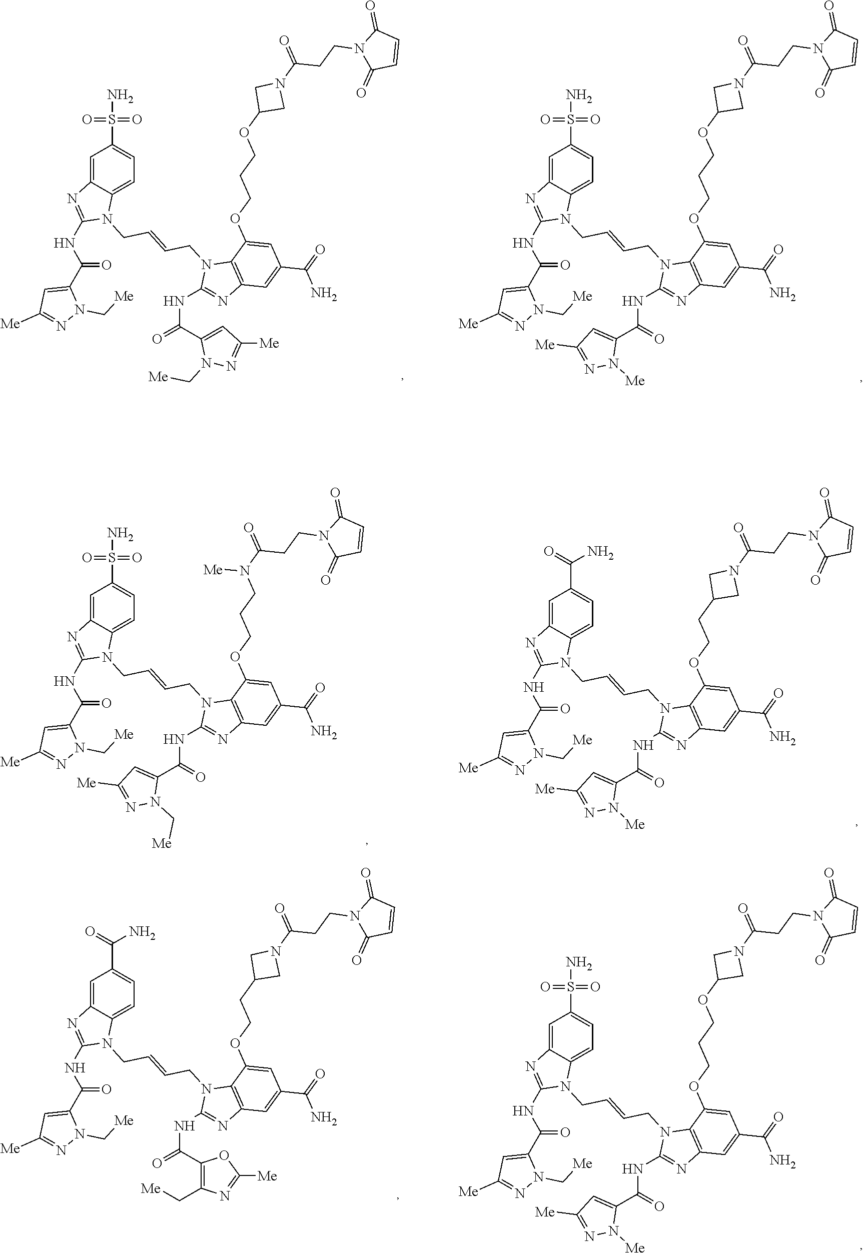 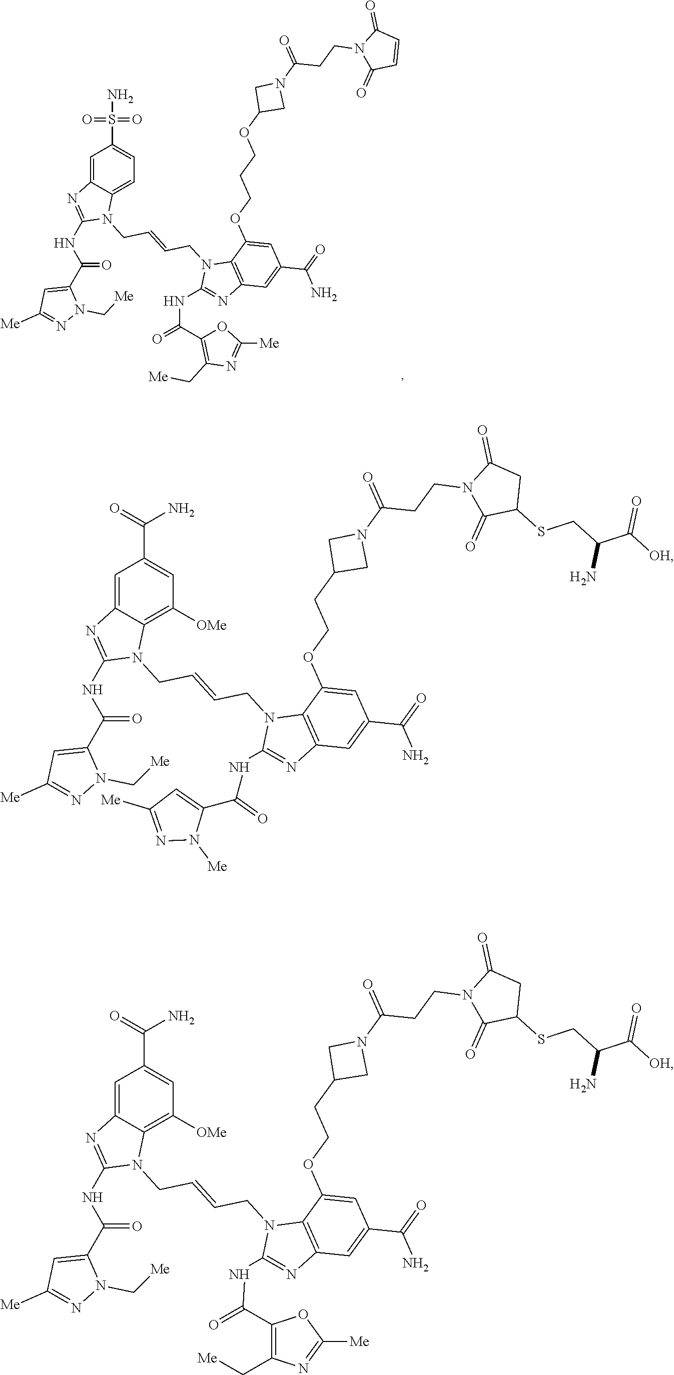 [SMILES: CCc1nc(C)oc1C(=O)Nc1nc2cc(C(N)=O)cc(OCCCOC3CN(C(=O)CCN4C(=O)C=CC4=O)C3)c2n1C/C=C/Cn1c(NC(=O)c2cc(C)nn2CC)nc2cc(S(N)(=O)=O)ccc21 ; CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCC3CN(C(=O)CCN4C(=O)CC(SC[C@H](N)C(=O)O)C4=O)C3)c21 ; CCc1nc(C)oc1C(=O)Nc1nc2cc(C(N)=O)cc(OCCC3CN(C(=O)CCN4C(=O)CC(SC[C@H](N)C(=O)O)C4=O)C3)c2n1C/C=C/Cn1c(NC(=O)c2cc(C)nn2CC)nc2cc(C(N)=O)cc(OC)c21] 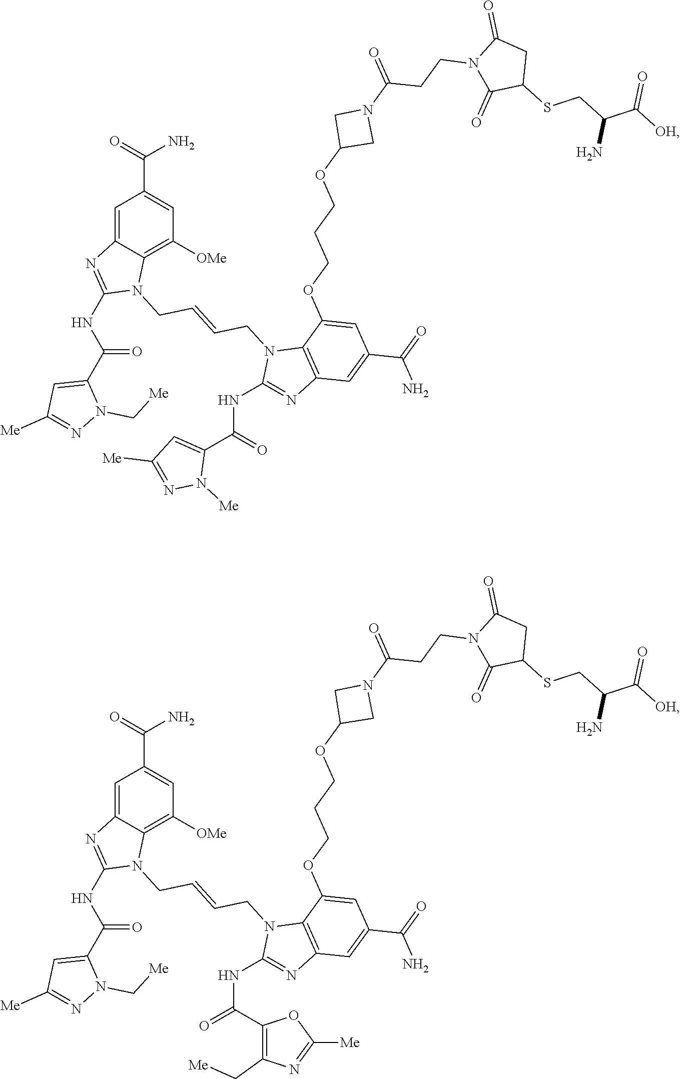 [SMILES: CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCCOC3CN(C(=O)CCN4C(=O)CC(SC[C@H](N)C(=O)O)C4=O)C3)c21 ; CCc1nc(C)oc1C(=O)Nc1nc2cc(C(N)=O)cc(OCCCOC3CN(C(=O)CCN4C(=O)CC(SC[C@H](N)C(=O)O)C4=O)C3)c2n1C/C=C/Cn1c(NC(=O)c2cc(C)nn2CC)nc2cc(C(N)=O)cc(OC)c21]
and pharmaceutically acceptable salts thereof.

378. A compound having the structure of Formula (V): 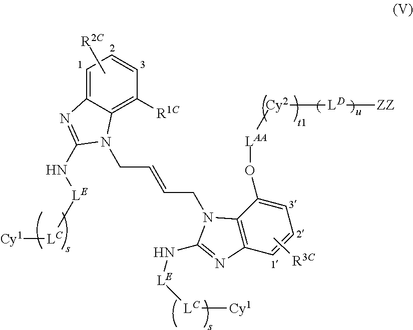
or a pharmaceutically acceptable salt thereof, wherein:
R^{1C} is hydrogen, hydroxyl, C₁₋₆ alkoxy, —(C₁₋₆ alkyl) C₁₋₆ alkoxy, —(CH₂)ₙ—NR^{A}R^{B}, or PEG2 to PEG4;
R^{2C} is —CO₂R^{M}, —(C═O)NR^{C}R^{D}, S(O)₂NR^{C}R^{D}, S(O)₂R^{M}, —(CH₂)_{q}—NR^{E}R^{F}, —(CH₂)_{q}—OR^{M}, —O(C═O)—NR^{E}R^{F}, or —NR^{M}(C═O)—NR^{E}R^{F}, wherein R^{2C} is attached at any one of positions labeled 1, 2, or 3;
R^{3C} is —CO₂R^{M}, —(C═O)NR^{C}R^{D}, S(O)₂NR^{C}R^{D}, S(O)₂R^{M}, —(CH₂)_{q}—NR^{E}R^{F}, —(CH₂)_{q}—OR^{M}, —O(C═O)—NR^{E}R^{F}, or —NR^{M}(C═O)—NR^{E}R^{F}, wherein R^{3C} is attached at any one of positions labeled 1′, 2′, or 3′;
each R^{A}, R^{B}, R^{C}, R^{D}, R^{E}, R^{F}, and R^{M} are independently hydrogen or C₁₋₆ alkyl;
each subscript n is independently an integer from 0 to 6;
each subscript q is independently an integer from 0 to 6;
L^{E} is —(C═O)— or —S(O)₂—;
L^{C} is —(CR^{I}R^{J})₁₋₃—
each R^{I} and R^{J} are independently hydrogen or C₁₋₃ alkyl;
subscript s is 0 or 1;
each Cy¹ is independently a 4-6 membered heterocycle, a 5-6 membered heteroaryl, or a C₃₋₆ cycloalkyl, each optionally substituted with one or more R^{K};
each R^{K} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d2}R^{e2}, —C(O)NR^{d2}R^{e2}, —C(O)(C₁₋₆ alkyl), and —C(O)O(C₁₋₆ alkyl);
each R^{d} and R^{e2} are independently hydrogen or C₁₋₃ alkyl;
L^{AA} is —(CH₂)₁₋₆—, —C(O)(CH₂)₁₋₆—, —C(O)NR^{L}(CH₂)₁₋₆—, —(CH₂)₁₋₆O—, —C(O)(CH₂)₁₋₆O—, or —C(O)NR^{L}(CH₂)₁₋₆O—;
R^{L} is hydrogen or C₁₋₃ alkyl;
Cy² is C₃₋₆ cycloalkyl, 4-6 membered heterocycle, 5-6 membered heteroaryl, or phenyl, each optionally substituted with one or more R^{U};
each R^{U} is independently selected from the group consisting of —CO₂R^{j1}, —(C═O)NR^{d3}R^{e3}, —S(O)₂NR^{d3}R^{e3}, —(CH₂)_{q1}—NR^{g1}R^{h1}, —(CH₂)_{q1}—OR^{j1}, and —(CH₂)_{q1}—(OCH₂CH₂)₁₋₈OH;
each R^{d3}, R^{e3}, R^{g1}, R^{h1}, and R^{j1} are independently hydrogen or C₁₋₆ alkyl;
subscript q1 is an integer from 0 to 6;
subscript t1 is 0 or 1;
L^{D} is —(CH₂)₁₋₆—;
subscript u is 0 or 1;
when t1 is 0, ZZ is —NR^{Q}R^{R}, —N⁺(C₁₋₆ alkyl)R^{Q}R^{R}, —C(═O)N^{S}R^{T}, —C(O)O(C₁₋₆ alkyl), —CO₂H, or an amino acid, or when t1 is 1, ZZ is hydrogen, —NR^{Q}R^{R}, —N⁺(C₁₋₆ alkyl)R^{Q}R^{R}; —C(═O)N^{S}R^{T}, —C(O)O(C₁₋₆ alkyl), —CO₂H, or an amino acid;
R^{Q} is hydrogen, C₁₋₆ alkyl, C₃₋₆ cycloalkyl, —(CH₂)₁₋₃C₃₋₆ cycloalkyl, —(CH₂)₁₋₃C₁₋₃ alkoxy, —(CH₂)₁₋₃ 4-6 membered heterocycle, or —(CH₂)₁₋₃ 5-6 membered heteroaryl, provided that
if t1 is 0 and both Cy¹ are 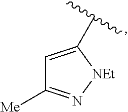
then R^{Q} is C₂₋₆ alkyl, C₃₋₆ cycloalkyl, —(CH₂)₁₋₃C₃₋₆ cycloalkyl, —(CH₂)₁₋₃C₁₋₃ alkoxy, —(CH₂)₁₋₃ 4-6 membered heterocycle, or —(CH₂)₁₋₃ 5-6 membered heteroaryl, and
if t1 is 0 and at least one Cy¹ is not 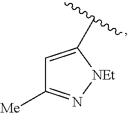
then ZZ is —NR^{Q}R^{R}, —N⁺(C₁₋₆ alkyl)R^{Q}R^{R}, or —C(═O)N^{S}R^{T}, and R^{Q} is C₁₋₆ alkyl, C₃₋₆ cycloalkyl, —(CH₂)₁₋₃C₃₋₆cycloalkyl, —(CH₂)₁₋₃C₁₋₃ alkoxy, —(CH₂)₁₋₃ 4-6 membered heterocycle, or —(CH₂)₁₋₃ 5-6 membered heteroaryl; and
each R^{R}, R^{S}, and R^{T} are independently hydrogen or C₁₋₆ alkyl.

379-507. (canceled)

508. The compound of claim 378, selected from the group consisting of: 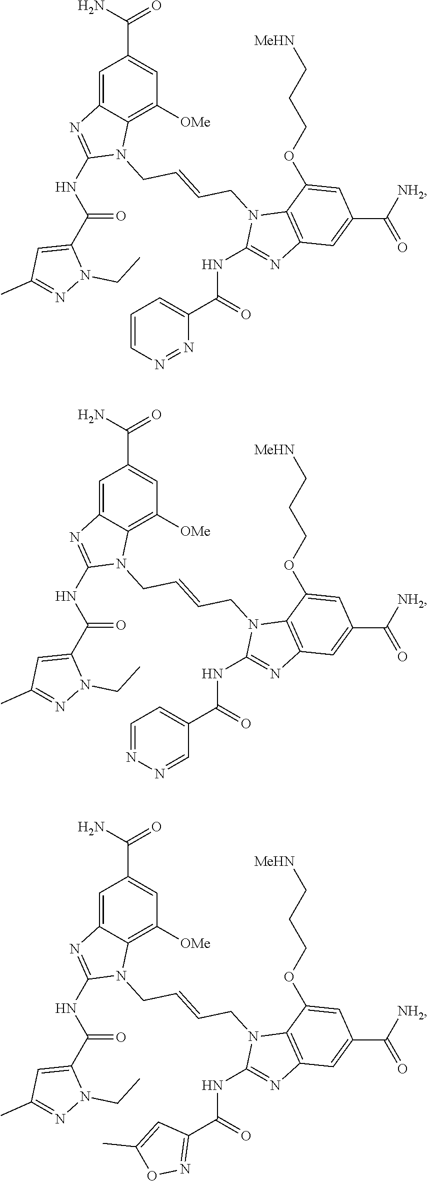 [SMILES: CCn1nc(C)cc1C(=O)Nc1nc2cc(C(N)=O)cc(OC)c2n1C/C=C/Cn1c(NC(=O)c2cccnn2)nc2cc(C(N)=O)cc(OCCCNC)c21 ; CCn1nc(C)cc1C(=O)Nc1nc2cc(C(N)=O)cc(OC)c2n1C/C=C/Cn1c(NC(=O)c2ccnnc2)nc2cc(C(N)=O)cc(OCCCNC)c21 ; CCn1nc(C)cc1C(=O)Nc1nc2cc(C(N)=O)cc(OC)c2n1C/C=C/Cn1c(NC(=O)c2cc(C)on2)nc2cc(C(N)=O)cc(OCCCNC)c21] 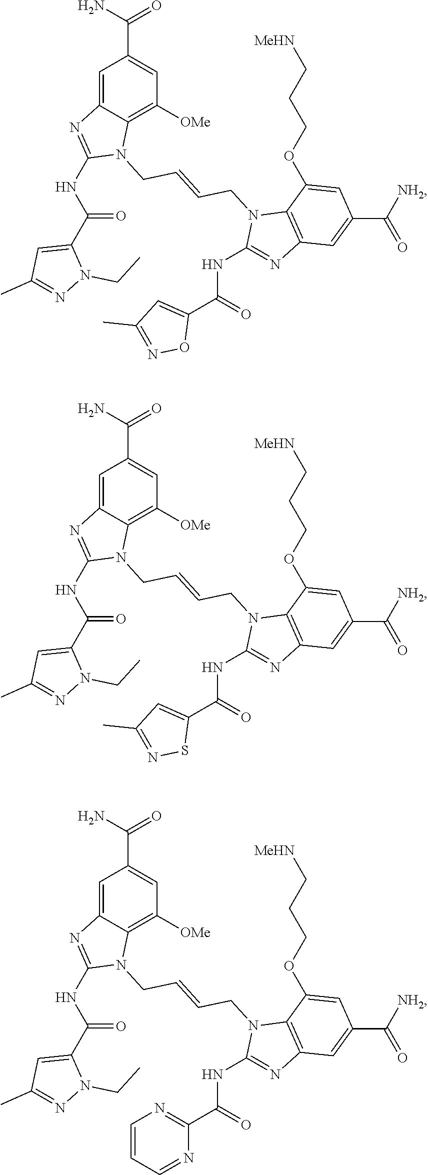 [SMILES: CCn1nc(C)cc1C(=O)Nc1nc2cc(C(N)=O)cc(OC)c2n1C/C=C/Cn1c(NC(=O)c2cc(C)no2)nc2cc(C(N)=O)cc(OCCCNC)c21 ; CCn1nc(C)cc1C(=O)Nc1nc2cc(C(N)=O)cc(OC)c2n1C/C=C/Cn1c(NC(=O)c2cc(C)ns2)nc2cc(C(N)=O)cc(OCCCNC)c21 ; CCn1nc(C)cc1C(=O)Nc1nc2cc(C(N)=O)cc(OC)c2n1C/C=C/Cn1c(NC(=O)c2ncccn2)nc2cc(C(N)=O)cc(OCCCNC)c21] 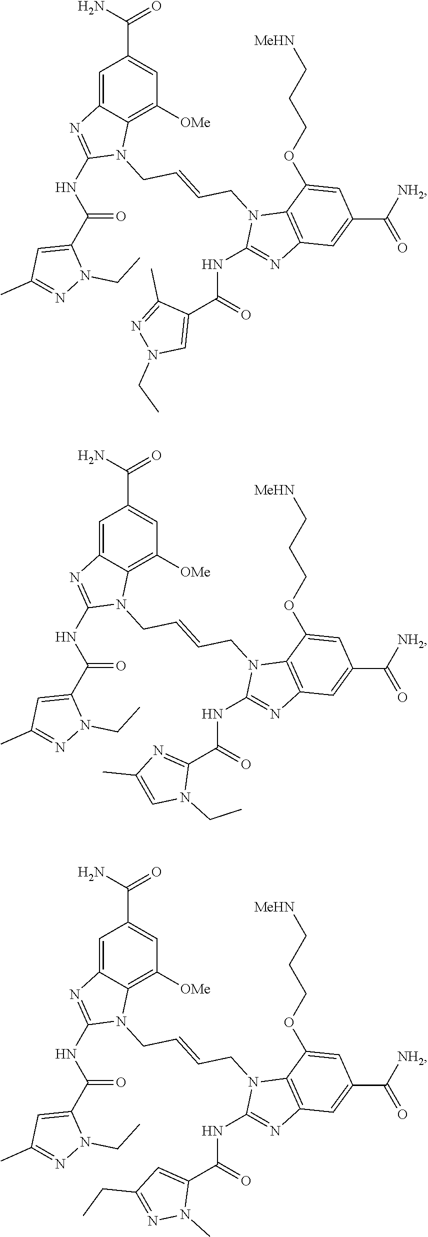 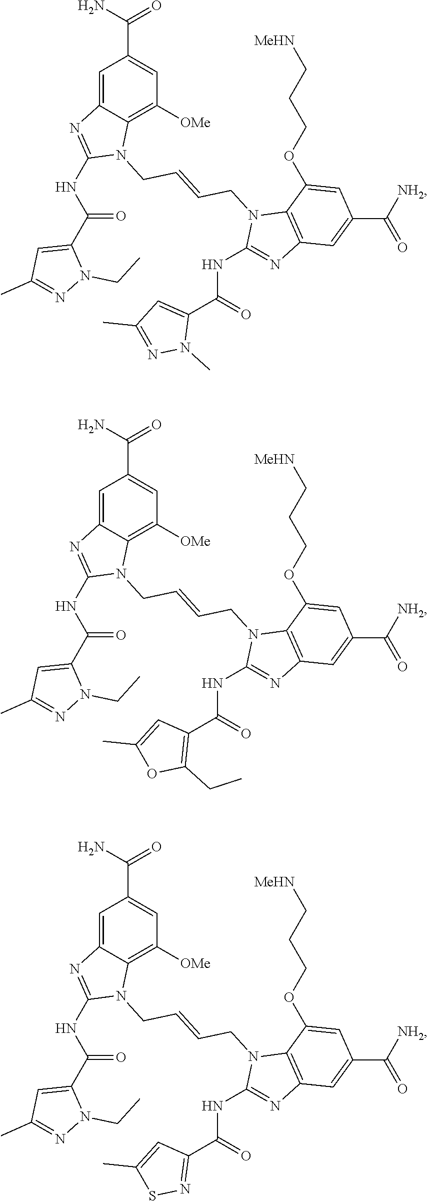 [SMILES: CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCCNC)c21 ; CCc1oc(C)cc1C(=O)Nc1nc2cc(C(N)=O)cc(OCCCNC)c2n1C/C=C/Cn1c(NC(=O)c2cc(C)nn2CC)nc2cc(C(N)=O)cc(OC)c21 ; CCn1nc(C)cc1C(=O)Nc1nc2cc(C(N)=O)cc(OC)c2n1C/C=C/Cn1c(NC(=O)c2cc(C)sn2)nc2cc(C(N)=O)cc(OCCCNC)c21] 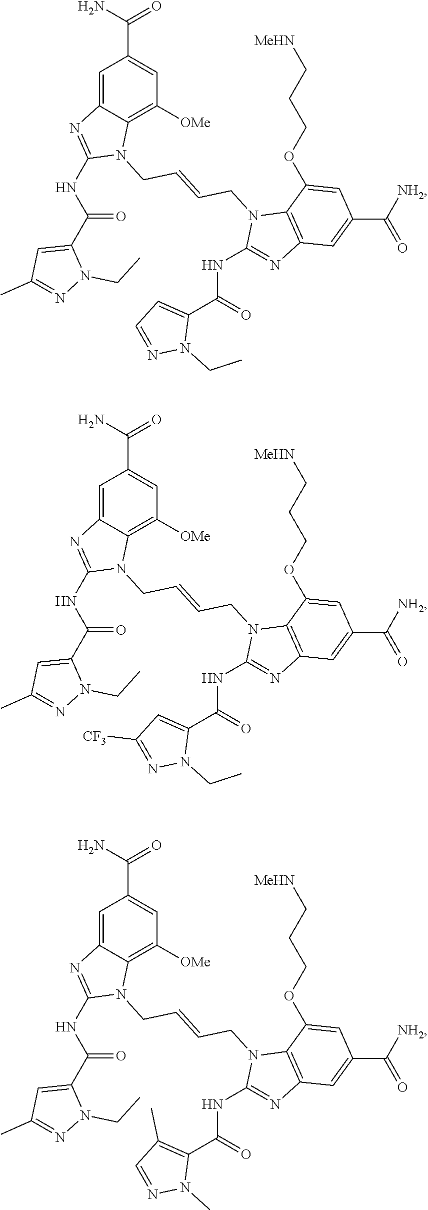 [SMILES: CCn1nc(C)cc1C(=O)Nc1nc2cc(C(N)=O)cc(OC)c2n1C/C=C/Cn1c(NC(=O)c2ccnn2CC)nc2cc(C(N)=O)cc(OCCCNC)c21 ; CCn1nc(C)cc1C(=O)Nc1nc2cc(C(N)=O)cc(OC)c2n1C/C=C/Cn1c(NC(=O)c2cc(C(F)(F)F)nn2CC)nc2cc(C(N)=O)cc(OCCCNC)c21 ; CCn1nc(C)cc1C(=O)Nc1nc2cc(C(N)=O)cc(OC)c2n1C/C=C/Cn1c(NC(=O)c2c(C)cnn2C)nc2cc(C(N)=O)cc(OCCCNC)c21] 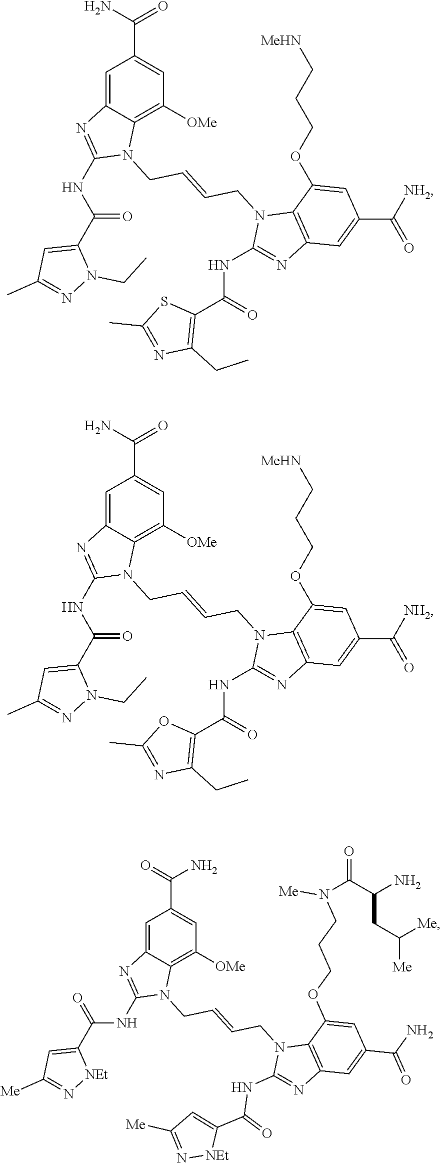 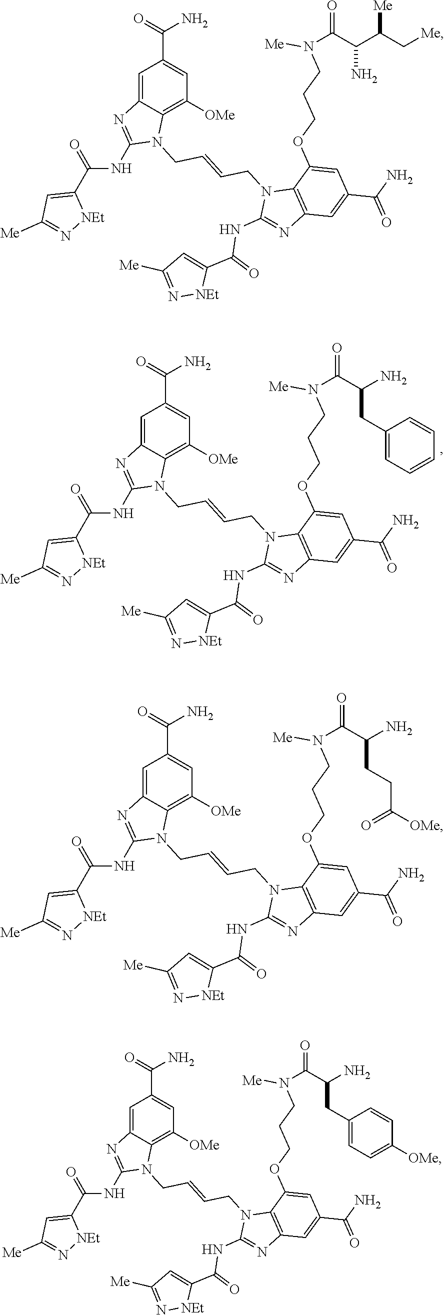 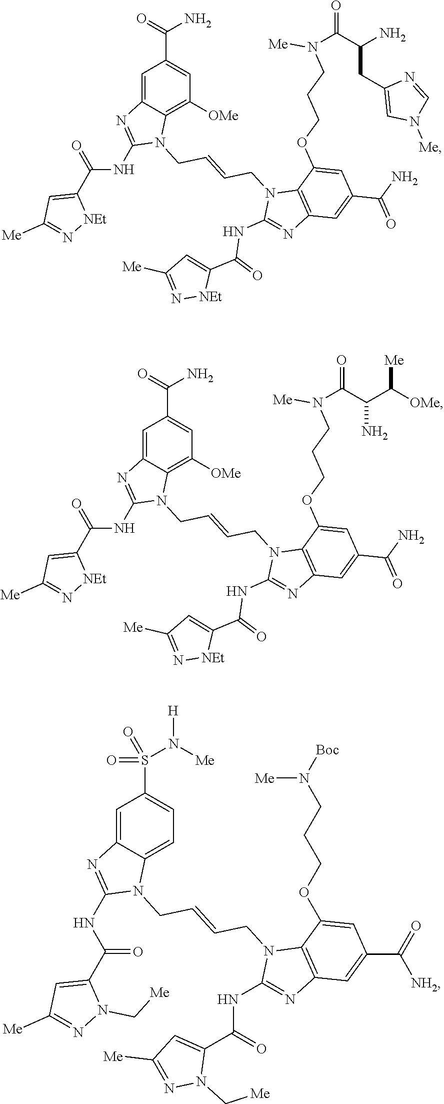 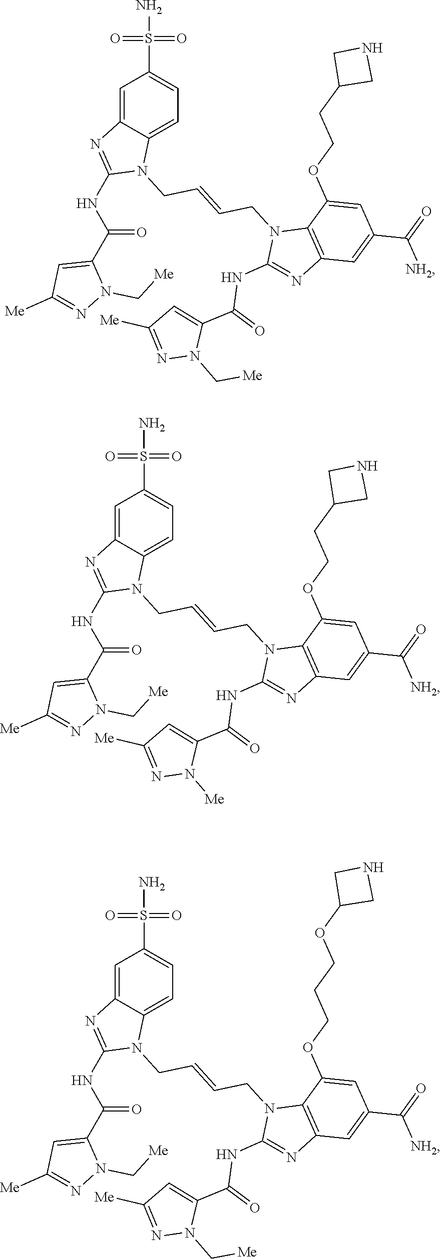 [SMILES: CCn1nc(C)cc1C(=O)Nc1nc2cc(S(N)(=O)=O)ccc2n1C/C=C/Cn1c(NC(=O)c2cc(C)nn2CC)nc2cc(C(N)=O)cc(OCCC3CNC3)c21 ; CCn1nc(C)cc1C(=O)Nc1nc2cc(S(N)(=O)=O)ccc2n1C/C=C/Cn1c(NC(=O)c2cc(C)nn2C)nc2cc(C(N)=O)cc(OCCC3CNC3)c21 ; CCn1nc(C)cc1C(=O)Nc1nc2cc(S(N)(=O)=O)ccc2n1C/C=C/Cn1c(NC(=O)c2cc(C)nn2CC)nc2cc(C(N)=O)cc(OCCCOC3CNC3)c21] 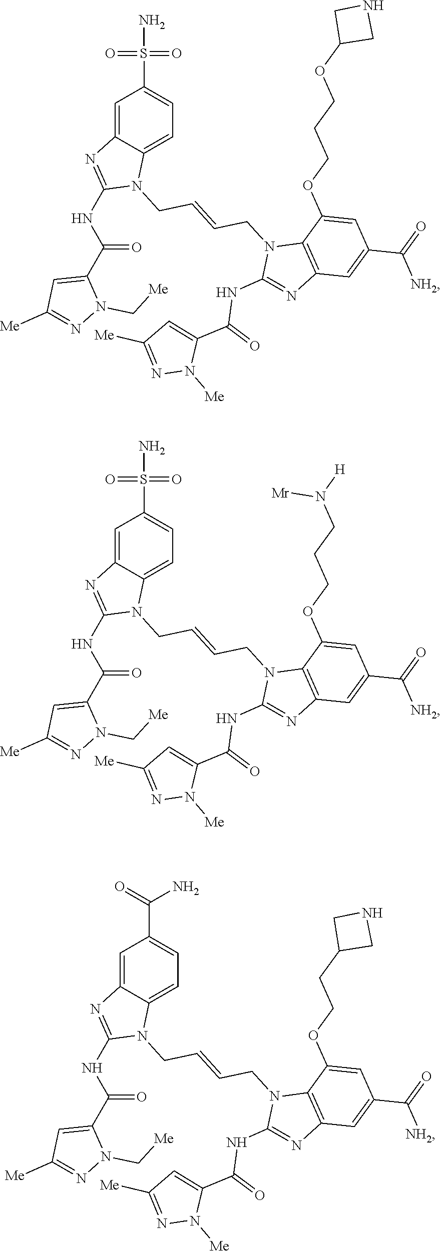 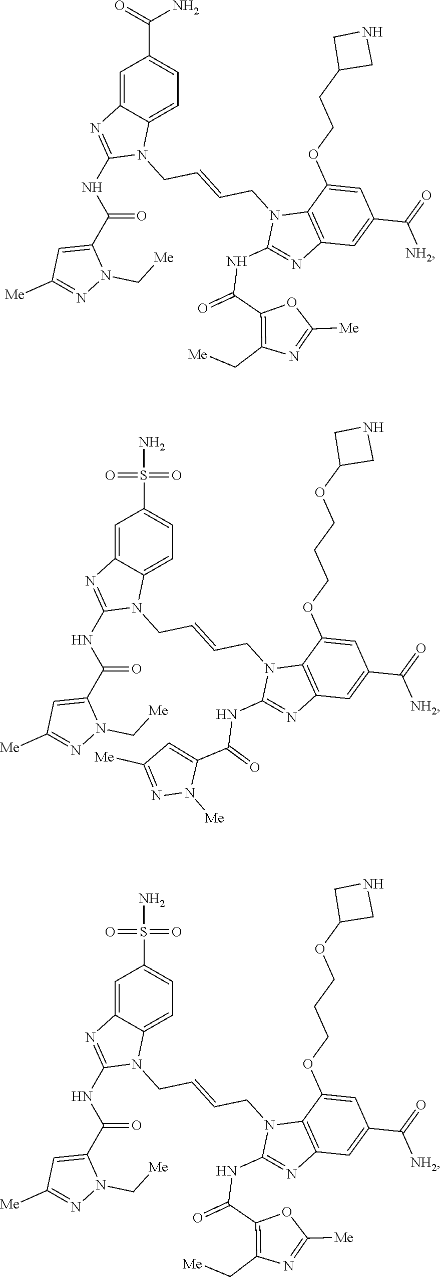 [SMILES: CCc1nc(C)oc1C(=O)Nc1nc2cc(C(N)=O)cc(OCCC3CNC3)c2n1C/C=C/Cn1c(NC(=O)c2cc(C)nn2CC)nc2cc(C(N)=O)ccc21 ; CCn1nc(C)cc1C(=O)Nc1nc2cc(S(N)(=O)=O)ccc2n1C/C=C/Cn1c(NC(=O)c2cc(C)nn2C)nc2cc(C(N)=O)cc(OCCCOC3CNC3)c21 ; CCc1nc(C)oc1C(=O)Nc1nc2cc(C(N)=O)cc(OCCCOC3CNC3)c2n1C/C=C/Cn1c(NC(=O)c2cc(C)nn2CC)nc2cc(S(N)(=O)=O)ccc21]
and pharmaceutically acceptable salts thereof.

509. An antibody-drug conjugate (ADC) having the formula:
Ab-(S*-(D′))ₚ
wherein:
Ab is an antibody;
each S* is a sulfur atom from a cysteine residue of the antibody;
D′ is a drug unit that is a radical of the compound of Formula (IV) according to claim 216; and
subscript p is an integer from 2 to 8.

510. The ADC of claim 509, wherein the radical of the compound of Formula (IV) comprises a radical in substituent M.

511. The ADC of claim 510, wherein D′ has the structure: 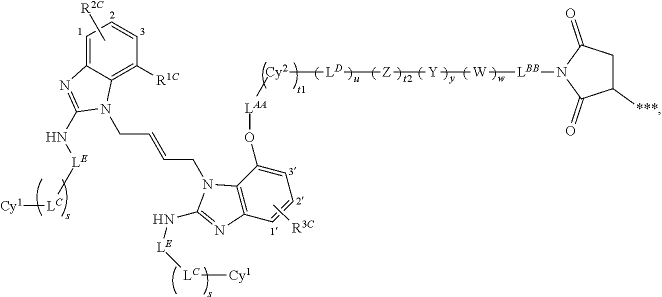
where *** indicates attachment to S*.

512-515. (canceled)

516. A composition comprising a distribution of the ADCs of claim 509.

517. The composition of claim 516, further comprising and at least one pharmaceutically acceptable carrier.

518. A method of treating cancer in a subject in need thereof, comprising administering a therapeutically effective amount of the composition of claim 516, to the subject.

519. A method of treating cancer in a subject in need thereof, comprising administering a therapeutically effective amount of the ADC of claim 509, to the subject.

520. A method of inducing an anti-tumor immune response in a subject in need thereof, comprising administering a therapeutically effective amount of the composition of claim 516, to the subject.

521. A method of inducing an anti-tumor immune response in a subject in need thereof, comprising administering a therapeutically effective amount of the ADC of claim 509, to the subject.

522. A compound of Formula (III): 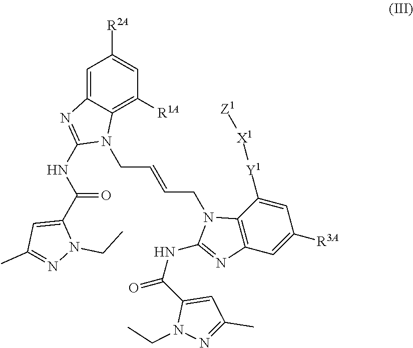
or a pharmaceutically acceptable salt thereof, wherein:
R^{1A} is hydrogen, hydroxyl, C₁₋₆ alkoxy, —(C₁₋₆ alkyl)C₁₋₆ alkoxy, —(CH₂)ₘ—NR^{AA}R^{BB};
R^{2A} and R^{3A} are independently —CO₂H, —(C═O)ₘₘ—NR^{CC}R^{DD}, or —(CH₂)_{q}—NR^{EE1}R^{FF1};
each subscript nn is independently an integer from 0 to 6;
each subscript mm is independently 0 or 1;
each subscript qq is an integer from 0 to 6;
Y¹ is —CH₂—, —O—, —S—, —NH—, or —N(CH₃)—;
X¹ is a C₂₋₆ alkylene;
Z¹ is —NR^{EE}R^{FF}, —C(═O)NR^{GG}R^{HH}, or —CO₂H;
each R^{AA}, R^{BB}, R^{CC}, and R^{DD}, R^{EE1}, and R^{FF1} are independently hydrogen or C₁₋₃ alkyl; and
each R^{EE}, R^{FF}, R^{GG}, and R^{HH} are independently hydrogen or C₁₋₆ alkyl.

523-579. (canceled)
